(19) United States
(12) Patent Application Publication
(10) Pub. No.: US 2026/0337188 A1
(43) Pub. Date: Oct. 29, 2026

(54) N-(5-SUBSTITUTED-[(1,3,4-THIADIAZOLYL) OR (1,3-THIAZOLYL)](SUBSTITUTED) CARBOXAMIDE COMPOUNDS, PHARMACEUTICAL COMPOSITIONS, AND METHODS OF PREPARING THE AMIDE COMPOUNDS AND OF THEIR USE

(71) Applicant: Gilead Sciences, Inc., Foster City, CA (US)

(72) Inventors: Bingcan Liu, Montreal (CA); Alexander Perryman, Pointe-Claire (CA); Philippe Mochirian, Montreal (CA); Michel Gallant, Pierrefonds (CA); David Bendahan, Cote Saint Luc (CA); Simon Surprenant, Boisbriand (CA); Janek Szychowski, Montreal (CA); Evelyne Dietrich, Laval (CA); Boubacar Sow, Vaudreuil-Dorion (CA); Monica Bubenik, Laval (CA); Stephen Morris, Beaconsfield (CA)

(21) Appl. No.: 19/702,770

(22) Filed: Jun. 9, 2026

Related U.S. Application Data
(63) Continuation of application No. 19/116,501, filed on Sep. 29, 2023.
(60) Provisional application No. 63/455,612, filed on Mar. 30, 2023.

(30) Foreign Application Priority Data
Sep. 29, 2022 (WO) PCT/CA2022/051446

(51) Int. Cl. C07D 417/12; A61K 31/4439; A61K 31/444; A61K 31/454; A61K 31/501; A61K 31/5377; A61K 31/55; A61K 45/06; C07D 417/14; C07D 487/04; C07D 491/04; C07D 491/107

(52) U.S. Cl. CPC C07D 417/12; A61K 31/4439; A61K 31/444; A61K 31/454; A61K 31/501; A61K 31/5377; A61K 31/55; A61K 45/06; C07D 417/14; C07D 487/04; C07D 491/04; C07D 491/107

(57) ABSTRACT

The present application discloses specific compounds of Formula (I) and their use in therapy, in particular, their use as Polymerase Theta (Polθ) inhibitors for the treatment of cancer. The application further discloses the use of a combination comprising a compound of Formula (I) and a radiotherapy or a radioligand in therapy, in particular, the use of specific compounds of Formula (I) as a Polymerase Theta (Polθ) inhibitor in combination with a radiotherapy or a radioligand for the treatment of cancer. (Formula I)

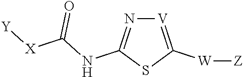

### FIELD OF THE INVENTION

The invention relates to compounds and pharmaceutical compositions, their preparation and their use in the treatment of a disease or condition. e.g., cancer, and, in particular, those diseases or conditions (e.g., cancers) which are dependent on the activity of human Polymerase Theta (Polθ) and/or have high cellular MMEJ/Theta mediated repair or Alternative End-joining repair.

### BACKGROUND

DNA damage occurs continually in cells as a result of environmental insults including ultraviolet radiation, X-rays, and endogenous stress factors, such as reactive oxygen and replicative stress. Cancer cells, in particular, are subject to a higher rate of DNA damage as a consequence of dysregulated DNA replication or as a consequence of anti-cancer therapy including irradiation or chemotherapy. Several DNA damage response pathways have evolved in a highly coordinated manner to help repair DNA damage and to act as a cellular checkpoint to stop the replication of cells with damaged DNA, allowing for repair functions to occur before the damaged DNA is passed on to daughter cells. Each of the identified DNA repair pathways sense and repair distinct but overlapping types of DNA damage. Double-strand breaks (DSBs), in which both strands in the double helix are severed, are particularly deleterious to the cell because they can lead to genome rearrangement and cell death. DSBs are repaired by homologous recombination (HR), classical nonhomologous end joining (cNHEJ), or by Polθ-mediated end joining (also known as alternative end joining (Alt-EJ) or microhomology mediate joining).

Polθ is a multifunctional enzyme composed of a superfamily 2 Hel308-type helicase domain at the N terminus, a low-fidelity A-family polymerase domain at the C terminus, and a non-structured central domain. The N-terminal helicase domain of polymerase theta is suggested to displace replication protein A and/or RAD51 molecules from 3′ single-stranded DNA to facilitate DNA synapsis at the microhomology sequences. Subsequently, Polθ-polymerase extends one end of the break by using the opposing strand of the other break end as a template. Polθ-polymerase can oscillate between templated and non-templated activities resulting in nucleotide insertions at alt-EJ repair junctions.

Polθ is also frequently overexpressed in human cancers and its overexpression is linked to poor prognosis in breast cancer. Furthermore, Polθ expression confers resistance to DSB-forming agents, including IR and chemotherapy drugs. Importantly, cancer cells that have defective HR or cNHEJ including BRCA1 or BRCA2 mutated breast and ovarian cancer cells become increasingly depend on Polθ for repair and survival. As a result, Polθ has emerged as a highly relevant cancer drug target. There is a need for new anti-cancer therapies and, in particular for Polθ Inhibitor-based anti-cancer therapies.

### SUMMARY OF THE INVENTION

In an aspect, the invention features a compound of formula (I):

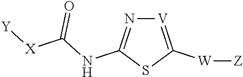

or a pharmaceutically acceptable salt thereof,
wherein
V is N or CR;
W is optionally substituted C₁₋₆ alkylene, C₁₋₆ alkoxyl, optionally substituted C₂₋₆ alkenyl, optionally substituted C₂₋₆ alkynyl, optionally substituted C₃₋₈ cycloalkyl, or optionally substituted C₆₋₁₀ aryl;
X is optionally substituted C₂₋₉ heterocyclylene, optionally substituted C₂₋₉ heteroarylene, or optionally substituted C₆₋₁₀ arylene, wherein X is further optionally substituted with -L¹-R^{X}, wherein L¹ is —O—, —NR^{X1}—, optionally substituted C₁₋₆ alkylene, optionally substituted C₁₋₆ heteroalkyl, optionally substituted C₂₋₆ alkenyl, optionally substituted allenyl, optionally substituted C₂₋₆ alkynyl, optionally substituted C₂₋₉ heterocyclylene, optionally substituted C₂₋₉ heteroarylene, or optionally substituted C₃₋₈ cycloalkylene, R^{X} is halo, amino, optionally substituted C₁₋₆ alkoxyl, optionally substituted acyl, carboxyl, amido, optionally substituted C₁₋₆ alkyl, optionally substituted C₂₋₆ heteroalkyl, optionally substituted C₂₋₉ heterocyclyl, optionally substituted C₂₋₉ heteroaryl, optionally substituted C₃₋₈ cycloalkyl C₁₋₆ alkyl, or optionally substituted C₂₋₉ heteroaryl C₁₋₆ alkyl, and R^{X1} is hydrogen or optionally substituted C₁₋₆ alkyl;
Y is optionally substituted C₂₋₉ heterocyclyl, optionally substituted C₂₋₉ heteroaryl, optionally substituted C₆₋₁₀ aryl;
Z is a H, halo, optionally substituted C₁₋₆ alkyl, optionally substituted C₂₋₆ alkynyl, optionally substituted C₁₋₆ alkoxy, optionally substituted C₃₋₈ cycloalkyl, optionally substituted C₂₋₉ heterocyclyl, optionally substituted C₂₋₉ heteroaryl, optionally substituted C₆₋₁₀ aryl, optionally substituted C₂₋₆ alkenyl, acyl, or amido; and
R is hydrogen, halogen, optionally substituted C₁₋₆ alkyl, CN, optionally substituted C₃₋₈ cycloalkyl, optionally substituted C₁₋₆ alkoxy, optionally substituted C₃₋₈ cycloalkoxy, N(R¹)₂, or C(O)NH₂, wherein each R¹ is independently hydrogen, optionally substituted C₁₋₆ alkyl, or optionally substituted C₃₋₈ cycloalkyl.

In some embodiments:
V is N or CR;
W is optionally substituted C₁₋₆ alkylene, optionally substituted C₂₋₆ alkenylene, optionally substituted C₂₋₆ alkynylene, optionally substituted C₃₋₈ cycloalkylene, or optionally substituted C₆₋₁₀ arylene;
X is optionally substituted C₂₋₉ heterocyclylene, optionally substituted C₂₋₉ heteroarylene, or optionally substituted C₆₋₁₀ arylene, wherein X is further optionally substituted with -L¹-R^{X}, wherein L¹ is —O—, —NR^{X1}—, optionally substituted C₂₋₉ heterocyclylene, optionally substituted C₂₋₉ heteroarylene, or optionally substituted C₃₋₈ cycloalkylene, R^{X} is optionally substituted C₁₋₆ alkyl, optionally substituted C₂₋₆ heteroalkyl, optionally substituted C₂₋₉ heterocyclyl, optionally substituted C₂₋₉ heteroaryl, optionally substituted C₃₋₈ cycloalkyl C₁₋₆ alkyl, or optionally substituted C₂₋₉ heteroaryl C₁₋₆ alkyl, and R^{X1} is hydrogen or optionally substituted C₁₋₆ alkyl;
Y is optionally substituted C₂₋₉ heterocyclyl, optionally substituted C₂₋₉ heteroaryl, optionally substituted C₆₋₁₀ aryl;
Z is a H, optionally substituted C₁₋₆ alkyl, optionally substituted C₂₋₆ alkynyl, optionally substituted C₁₋₆ alkoxy, optionally substituted C₃₋₈ cycloalkyl, optionally substituted C₂₋₉ heterocyclyl, optionally substituted C₂₋₉ heteroaryl, or optionally substituted C₆₋₁₀ aryl; and
R is hydrogen, halogen, optionally substituted C₁₋₆ alkyl, CN, optionally substituted C₃₋₈ cycloalkyl, optionally substituted C₁₋₆ alkoxy, optionally substituted C₃₋₈ cycloalkoxy, N(R¹)₂, or C(O)NH₂, wherein each R¹ is independently hydrogen, optionally substituted C₁₋₆ alkyl, or optionally substituted C₃₋₈ cycloalkyl.

In some embodiments:
V is N or CR;
W is optionally substituted C₁₋₆ alkylene, optionally substituted C₂₋₆ alkenylene, optionally substituted C₂₋₆ alkynylene, optionally substituted C₃₋₈ cycloalkylene, or optionally substituted C₂₋₁₀ arylene;
X is optionally substituted C₂₋₉ heterocyclylene, optionally substituted C₂₋₉ heteroarylene, or optionally substituted C₆₋₁₀ arylene;
L¹ is optionally substituted C₂₋₆ alkenyl, optionally substituted C₂₋₆ alkynyl, optionally substituted C₅-C₈ cycloalkyl, optionally substituted aryl, or optionally substituted heteroaryl;
Y is optionally substituted C₂₋₉ heterocyclyl, optionally substituted C₂₋₉ heteroaryl, optionally substituted C₆₋₁₀ aryl;
Z is a H, optionally substituted C₁₋₆ alkyl, optionally substituted C₂₋₆ alkynyl, optionally substituted C₁₋₆ alkoxy, optionally substituted C₃₋₈ cycloalkyl, optionally substituted C₂₋₉ heterocyclyl, optionally substituted C₂₋₉ heteroaryl, or optionally substituted C₆₋₁₀ aryl; and
R is hydrogen, halogen, optionally substituted C₁₋₆ alkyl, CN, optionally substituted C₃₋₈ cycloalkyl, optionally substituted C₁₋₆ alkoxy, optionally substituted C₃₋₈ cycloalkoxy, N(R¹)₂, or C(O)NH₂, wherein each R¹ is independently hydrogen, optionally substituted C₁₋₆ alkyl, or optionally substituted C₃₋₈ cycloalkyl.

In some embodiments:
V is N or CR;
W is optionally substituted C₁₋₆ alkylene, optionally substituted C₂₋₆ alkenylene, optionally substituted C₂₋₆ alkynylene, optionally substituted C₃₋₈ cycloalkylene, or optionally substituted C₆₋₁₀ arylene;
X is optionally substituted C₂₋₉ heterocyclylene, optionally substituted C₂₋₉ heteroarylene, or optionally substituted C₆₋₁₀ arylene;
Y is optionally substituted C₂₋₉ heterocyclyl, optionally substituted C₂₋₉ heteroaryl, optionally substituted C₆₋₁₀ aryl;
Z is a H, optionally substituted C₁₋₆ alkyl, optionally substituted C₁₋₆ alkoxy, optionally substituted C₃₋₈ cycloalkyl, optionally substituted C₂-s heterocyclyl, optionally substituted C₂₋₉ heteroaryl, or optionally substituted C₆₋₁₀ aryl; and
R is hydrogen, halogen, optionally substituted C₁₋₆ alkyl, CN, optionally substituted C₃₋₈ cycloalkyl, optionally substituted C₁₋₆ alkoxy, optionally substituted C₃₋₈ cycloalkoxy, N(R¹)₂, or C(O)NH₂, wherein each R¹ is independently hydrogen, optionally substituted C₁₋₆ alkyl, or optionally substituted C₃₋₈ cycloalkyl.

In some embodiments, W is ethylene, ethynylene, or cyclopropylene. In some embodiments, V is N.

In some embodiments, the compound is a compound of formula II:

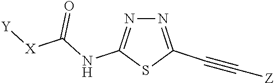

or a pharmaceutically acceptable salt thereof.

In some embodiments, the compound is a compound of formula (111):

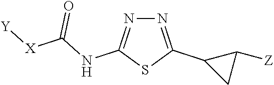

or a pharmaceutically acceptable salt thereof.

In some embodiments, V is CR. In some embodiments, V is CH.

In some embodiments, the compound is a compound of formula (IV):

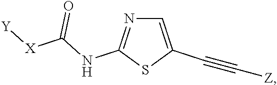

or a pharmaceutically acceptable salt thereof.

In some embodiments, the compound is a compound of formula (V):

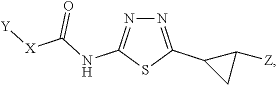

or a pharmaceutically acceptable salt thereof.

In some embodiments, X is optionally substituted 5- or 6-membered C₂₋₉ heterocyclylene, optionally substituted bicyclic C₂₋₉ heterocyclylene, or optionally substituted phenylene. In some embodiments, the valences of X are vicinal. In some embodiments, X is optionally substituted with one or two groups independently selected from the group consisting of halogen, CF₃, CN, C₃₋₄ cycloalkyl. C₁₋₆ alkyl, C₁₋₆ alkoxy. C₃₋₄ cycloalkoxy, N(R¹)₂, —(CH₂)ₚC(O)N(R¹)₂, —C≡C—R², and —(CH₂)_{q}-L-(R³), wherein each R¹ is independently H, C₁₋₆ alkyl, or C₃₋₄ cycloalkyl, p and q are each independently 0 or 1, R² is 4-hydroxyl-tetrahydropyran-4-yl, 3-hydroxy-oxetan-3-yl, L is 5-membered heteroarylene, and R³ is H or C₁₋₆ alkyl.

In some embodiments —X—Y is

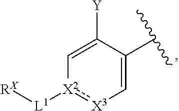

wherein [symbol] is a single bond, X² is N, and X³ is CO, or [symbol] is a double bond, X² is C, and X³ is N or CH.

In some embodiments, —X—Y is

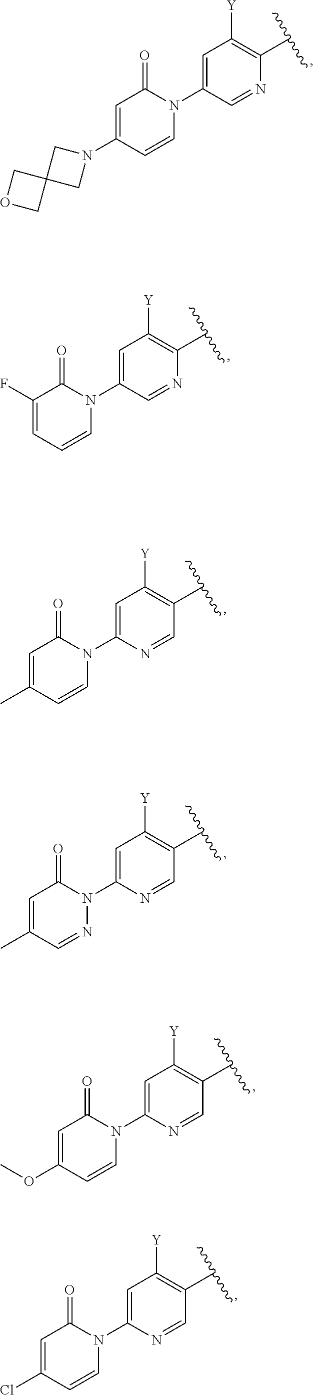

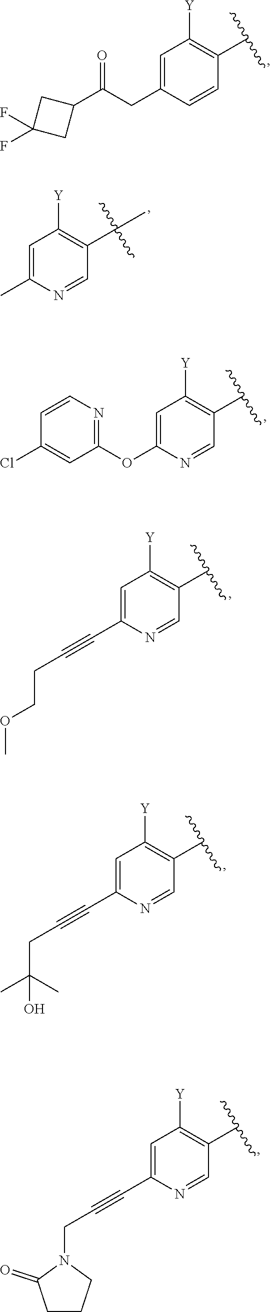

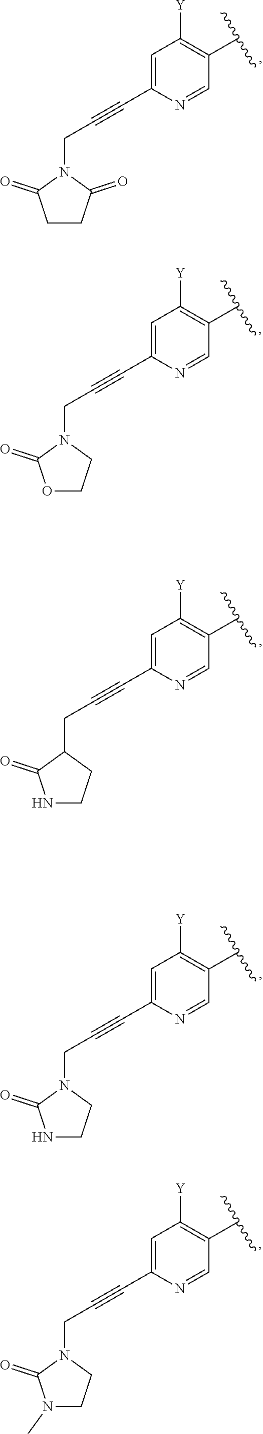

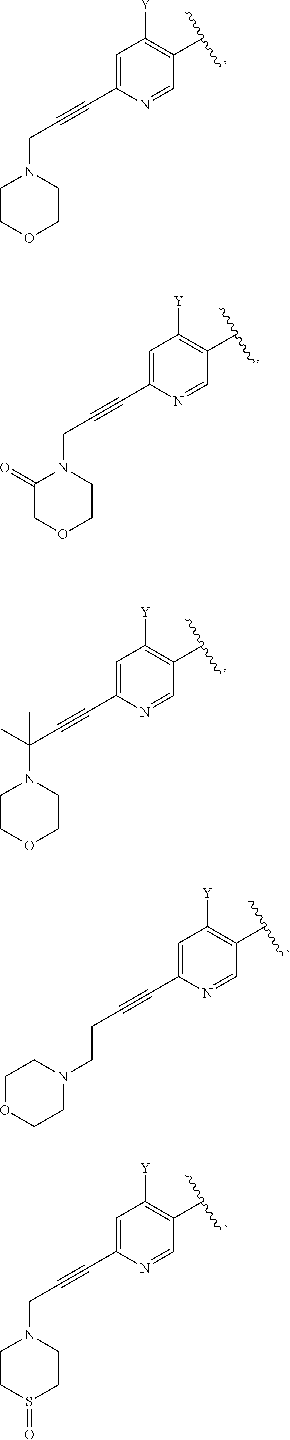

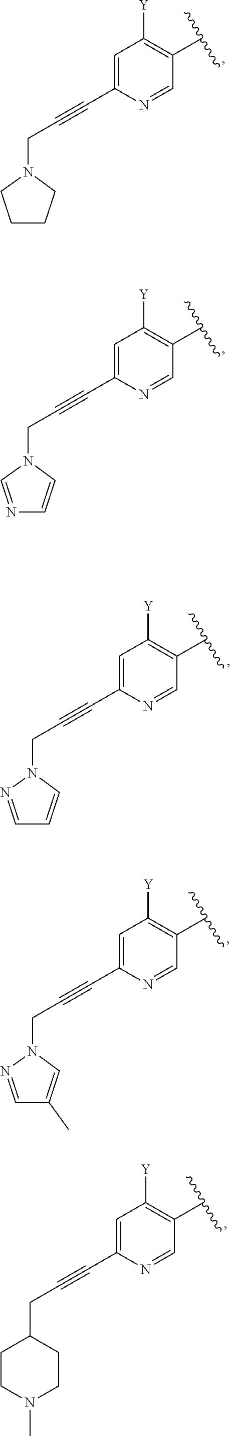

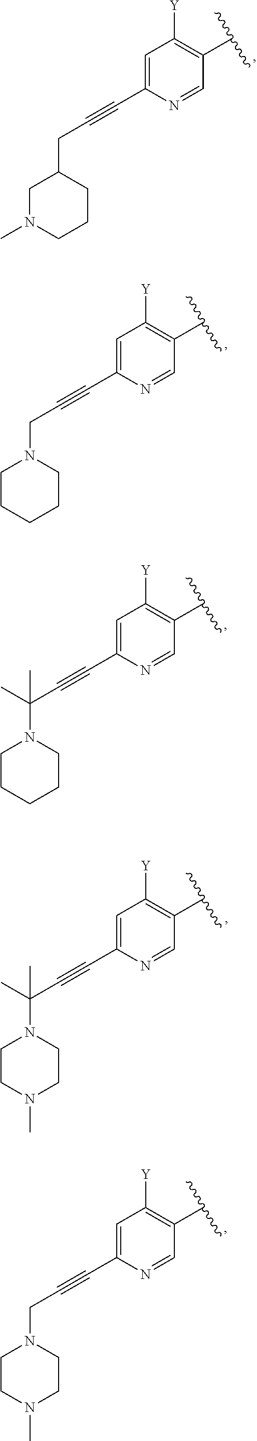

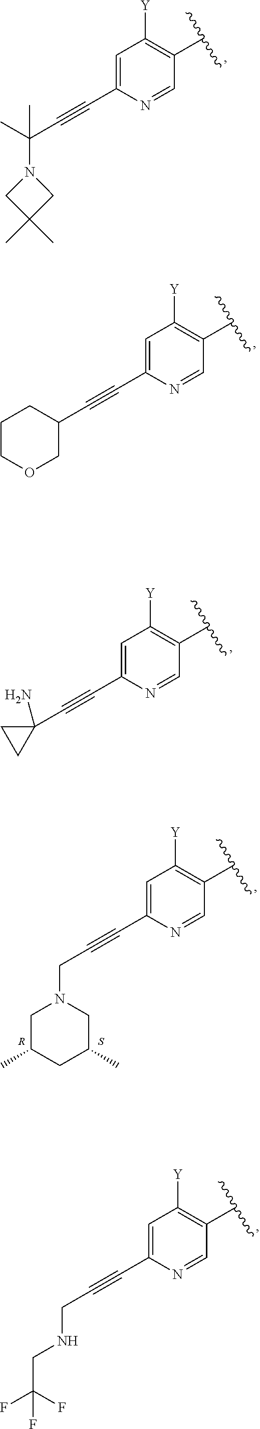

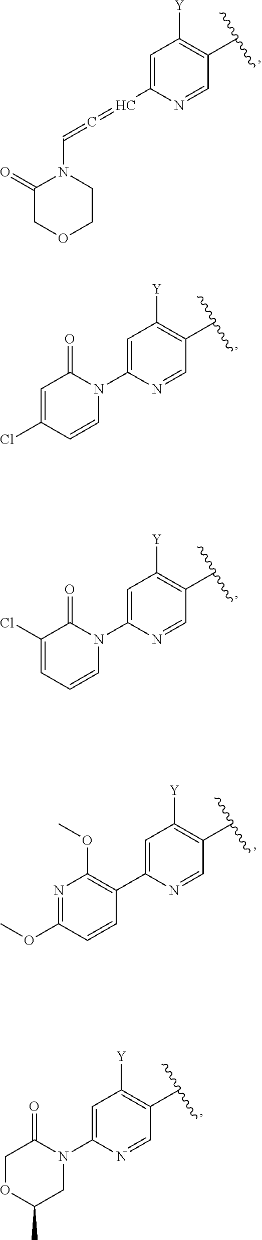

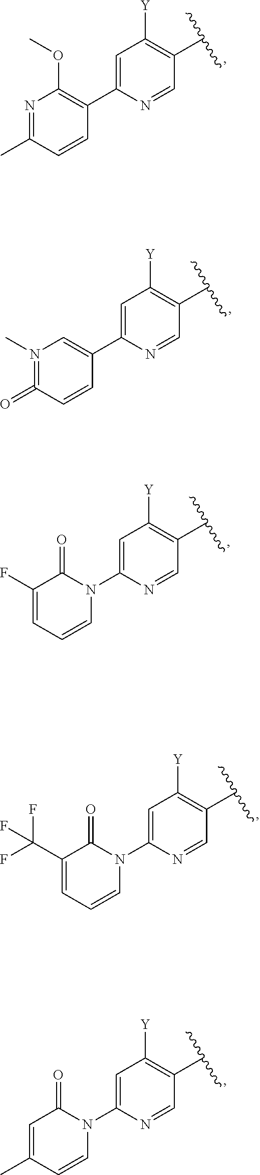

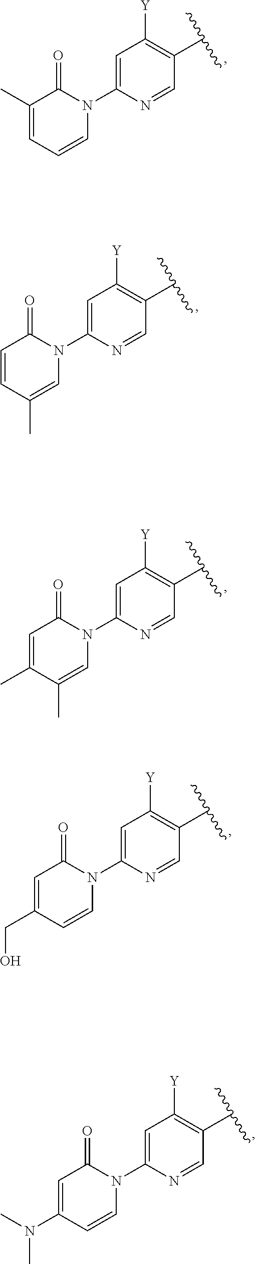

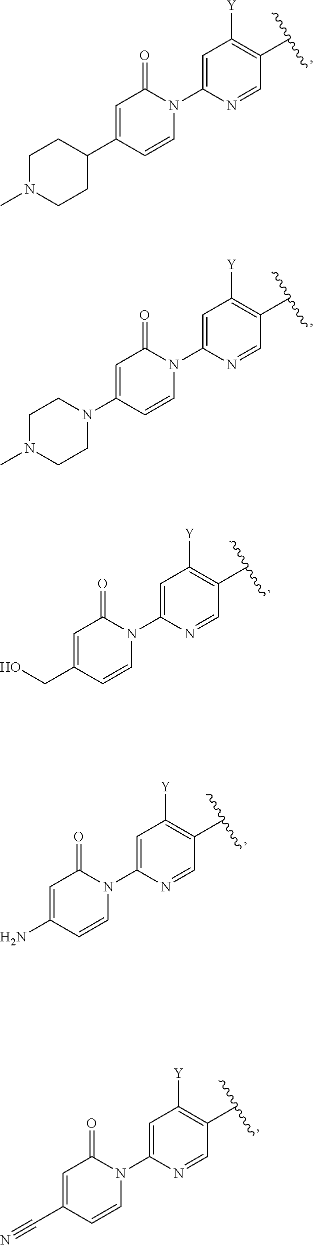

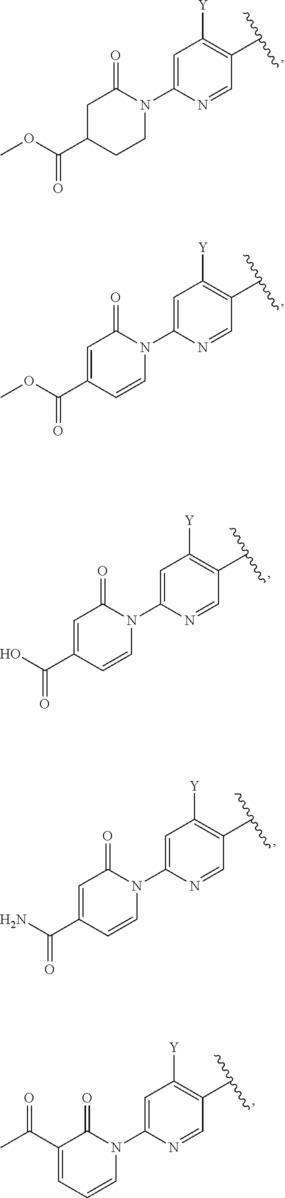

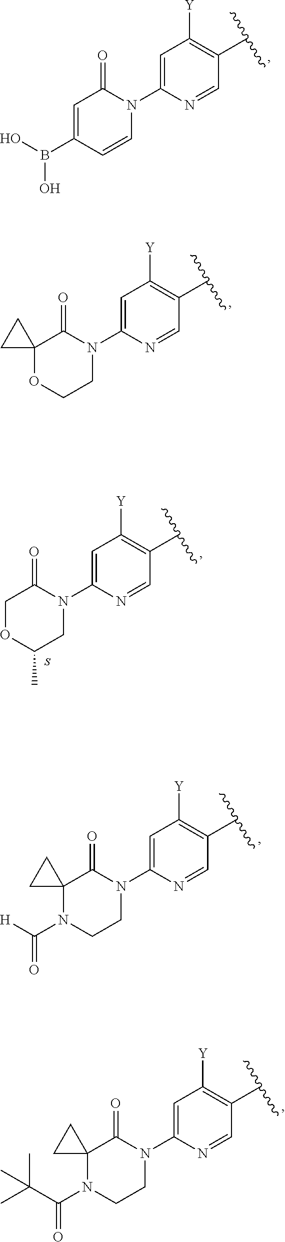

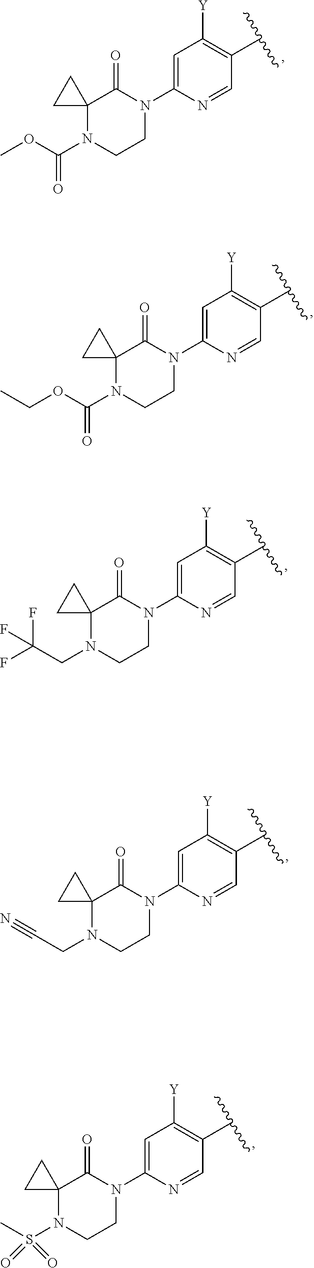

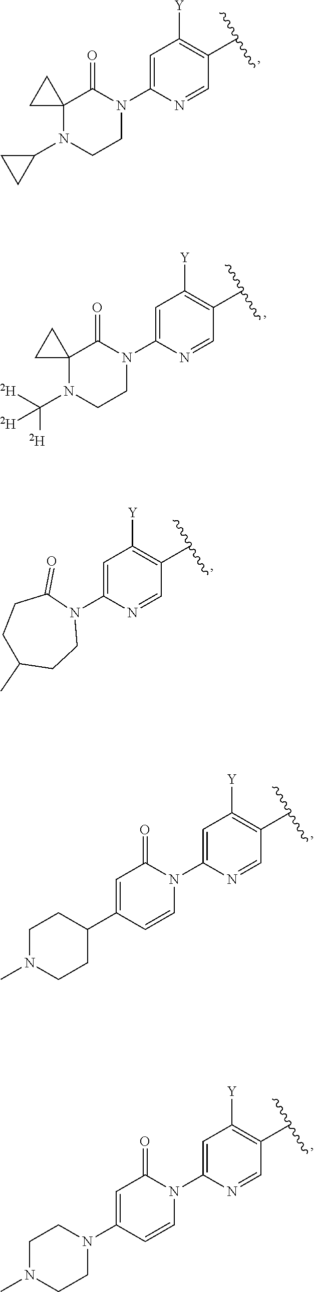

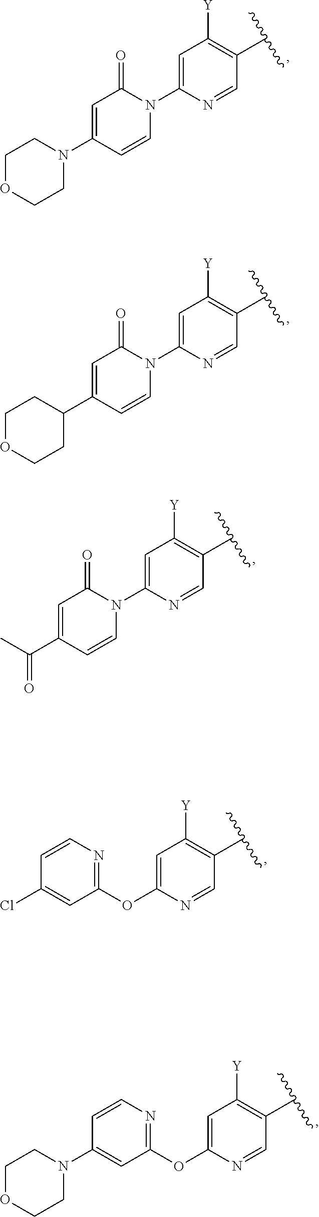

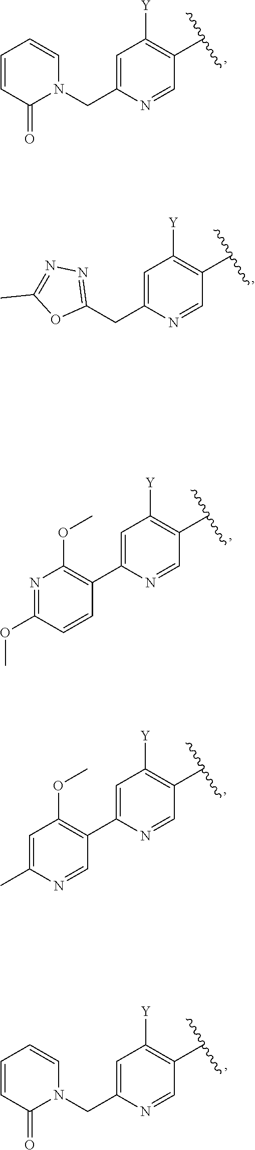

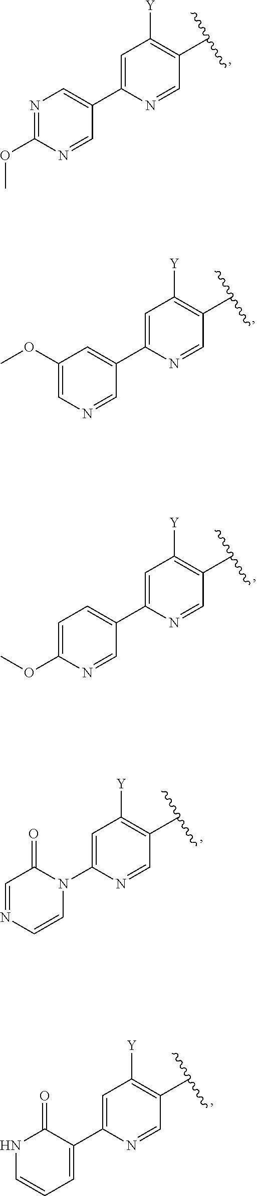

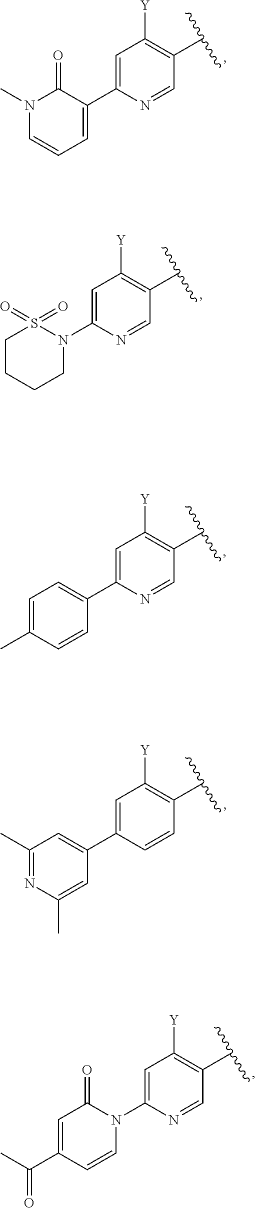

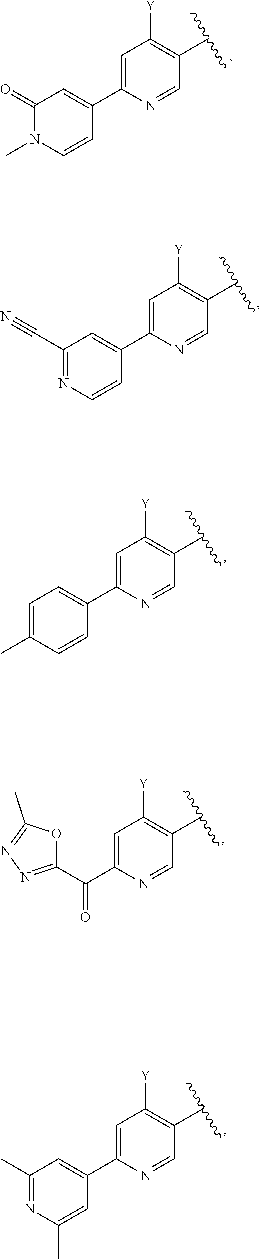

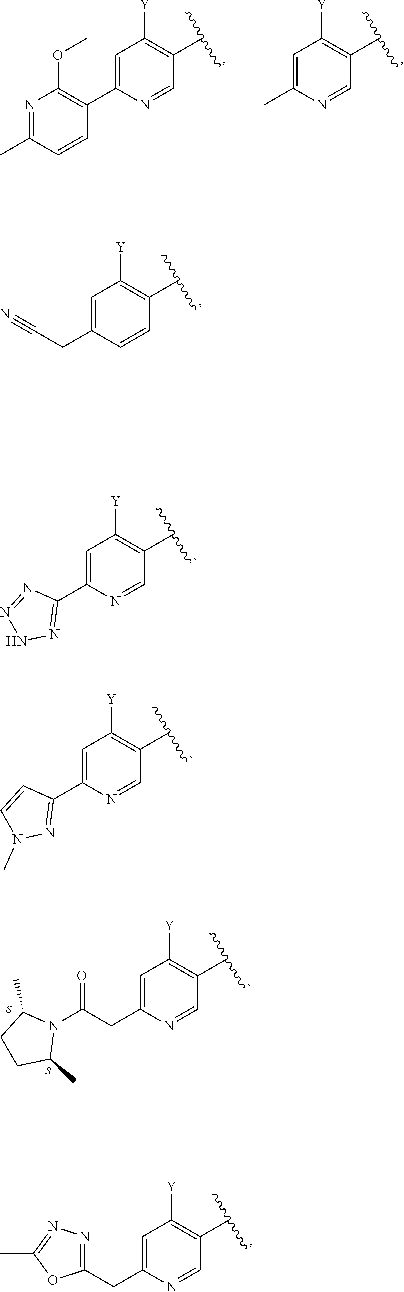

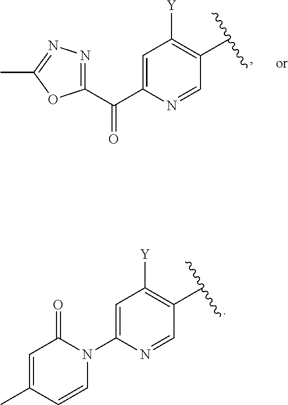

In some embodiments, Y is optionally substituted 5- or 6-membered C₂₋₉ heterocyclyl or optionally substituted phenyl. In some embodiments, Y is optionally substituted 5- or 6-membered C₂₋₉ heteroaryl or optionally substituted phenyl. In some embodiments, Y is optionally substituted pyridinyl or optionally substituted phenyl. In some embodiments, Y is optionally substituted with one, two, or three groups independently selected from the group consisting of halogen, CHF₂, CF₃, or C₁₋₆ alkoxy. In some embodiments, Y is

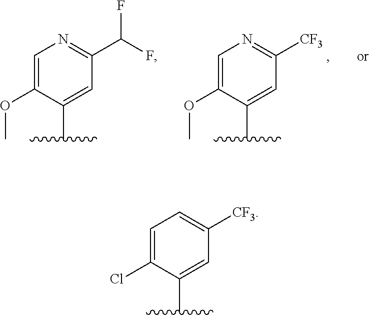 [SMILES: *c1cc(C(F)F)ncc1OC ; *c1cc(C(F)(F)F)ncc1OC ; *c1cc(C(F)(F)F)ccc1Cl]

In some embodiments, Z is H, halo, optionally substituted C₃₋₆ alkyl, optionally substituted C₃₋₈ cycloalkyl, optionally substituted C₂₋₉ heterocyclyl, or optionally substituted phenyl. In some embodiments, Z is H, optionally substituted C₃₋₆ alkyl, optionally substituted C₃₋₈ cycloalkyl, optionally substituted non-aromatic C₂₋₉ heterocyclyl, optionally substituted 5 or 6-membered C₂₋₉ heterocyclyl or optionally substituted phenyl. In some embodiments, Z is optionally substituted pyrazolyl, optionally substituted phenyl, optionally substituted cyclopropyl, optionally substituted cyclobutyl, optionally substituted spiro[3.3]heptyl, optionally substituted 1,3-thiazole, alkoxycarbonylamino, dialkylamino, optionally substituted methoxy, optionally substituted methyl, optionally substituted ethynyl.

In some embodiments, Z is optionally substituted with one, two, or three groups independently selected from the group consisting of halogen, CF₃, CN, C₃₋₄ cycloalkyl, C₁₋₆ alkyl, C₁₋₆ alkoxy. C₃₋₄ cycloalkoxy, N(R¹)₂, and C(O)NH₂, wherein each R¹ is independently H, C₁₋₆ alkyl, or C₃₋₄ cycloalkyl.

In some embodiments, Z is

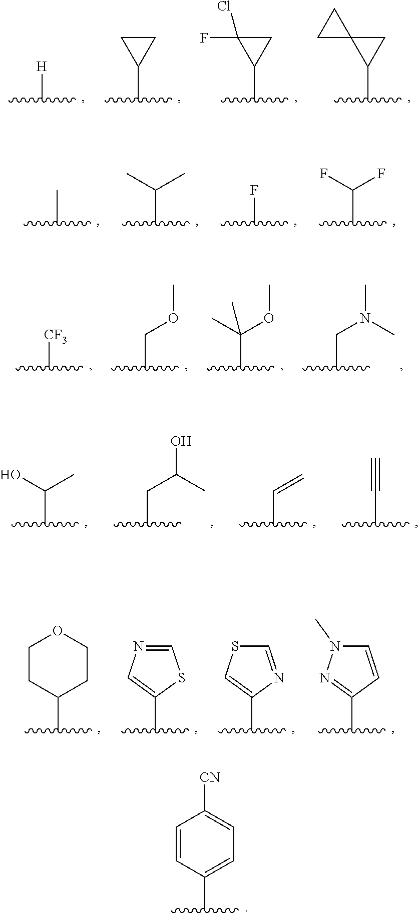

In some embodiments, at least one heterocyclyl includes pyridyl, pyrimidinyl, pyrazinyl, imidazolyl, pyrazolyl, thiazolyl, oxazolyl, oxadiazolyl, triazolyl, or pyridonyl. In some embodiments, at least one cycloalkyl includes cyclopropyl, cyclobutyl, cyclopentyl, or spiro[2.2]pentyl. In some embodiments, at least one heterocyclyl includes oxetanyl, tetrahydrofuryl, morpholinyl, piperidinyl, or piperazinyl. In some embodiments, at least one heterocyclyl includes indolyl, indazolyl, benzimidazolyl, benzothiazolyl, benzoxazolyl, imidazo[1,2-a]pyridyl, or quinolinyl.

In some embodiments, the compound is a compound of formula (VI):

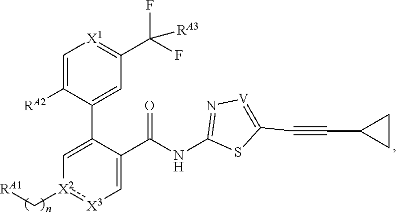

or a pharmaceutically acceptable salt thereof,
wherein
n is 0 or 1;
R^{A1} is a C₂-C₉ heteroaryl optionally substituted with C₁-C₆ alkyl or a C₄-C₉ heterocyclyl optionally substituted with oxo;
R^{A2} is a C₁-C₆ alkyl, C₁-C₆ alkoxy, or halogen;
R^{A3} is hydrogen or a halogen;
each of X¹ and V is independently N or CH; and
[symbol] is a single bond, X² is N, and X³ is CO, or [symbol] is a double bond, X² is C, and X³ is N or CH.

In some embodiments, V is CH. In some embodiments, V is N. In some embodiments, [symbol] is a single bond, X² is N, and X³ is CO. In some embodiments, [symbol] is a double bond, X² is C, and X³ is N. In some embodiments, [symbol] a double bond. X² is C, and X³ is CH. In some embodiments, R^{A}2 is C₁₋₆ alkoxy. In some embodiments, R^{A2} is methoxy. In some embodiments, R^{A3} is hydrogen. In some embodiments, R^{A1} is C₂-C₉ heteroaryl optionally substituted with C₁-C₆ alkyl. In some embodiments, the C₂-C₉ heteroaryl is optionally substituted with methyl. In some embodiments, the C₂-C₉ heteroaryl is a 5-membered heteroaryl. In some embodiments, the C₂-C₉ heteroaryl is a 6-membered heteroaryl.

In some embodiments, n is 0. In some embodiments, n is 1.

In some embodiments, the compound is selected from the group consisting of compounds 359-643 and pharmaceutically acceptable salts thereof.

In a second aspect, the disclosure features a pharmaceutical composition including the compound of the first and a pharmaceutically acceptable excipient. In some embodiments, the composition is isotopically enriched in deuterium.

In a third aspect, the disclosure features a method of inhibiting Polθ in a cell expressing Polθ, the method comprising contacting the cell with a compound selected from the group of compounds in Table 2, or a pharmaceutically acceptable salt thereof. In some embodiments, the cell is in a subject.

In a fourth aspect, the disclosure features a method of treating a subject in need thereof comprising administering to the subject a compound selected from Table 2, or a pharmaceutically acceptable salt thereof.

In some embodiments of the third or the fourth aspect, the method includes administering an additional anticancer therapy. In some embodiments, the additional anticancer therapy is a radiotherapy, a radioligand, an ADC, an immune checkpoint inhibitor, PARP inhibitor, DNA-PK inhibitor, an ATM inhibitor, an ATR inhibitor, a wee1 inhibitor, a PKMYT1 inhibitor, or a CHK1 inhibitor. In some embodiments, the additional anticancer therapy is a radiotherapy or a radioligand.

In a fifth aspect, the disclosure features a method of inhibiting Polθ in a cell expressing Polθ, the method comprising contacting the cell with a compound of Formula (I) (e.g., any one of compounds 1 to 643), or a pharmaceutically acceptable salt thereof, in combination with a radiotherapy or a radioligand. In some embodiments, the cell is in a subject.

In a sixth aspect, the disclosure features a method of treating a subject in need thereof comprising administering to the subject a compound of Formula (I) (e.g., any one of compounds 1 to 643), or a pharmaceutically acceptable salt thereof, in combination with a radiotherapy or a radioligand.

In some embodiments of the fifth or the sixth aspect, the compound is a compound selected from Table 1, or a pharmaceutically acceptable salt thereof.

In some embodiments of the third, fourth, fifth, or sixth aspect, the radioligand is selected from the group consisting of zevalin, actimab-A, lomab-ACT, lomab-B, lutetium-177-DOTAGA-PEG-IAC, tozaride, SS0110, BAY-2701439, ¹¹⁷Lu-rhPSMA-10.1, CTT-1403, iopofosine, SAR-BBN, SAR-bisPSMA, SARTATE, FAP-2286, CONV-01-α, ¹¹⁷Lu-PSMA-I&T, FPI-2059, FPI-1434, FPI-1966, [¹⁷⁷Lu] ludotadipep, ¹⁶¹Tb-PSMA-I&T, ITM-31, ITM-11, JNJ-69086420, I-¹³¹-1095, azedra, PSMA TTC/BAY-2315497, ¹¹⁷Lu-DOTA-EB-TATE, betalutin, AAA817, AAA603, lutathera, pluvicto, PPMX-T002, 186RNL, PNT2003, CAM−H2, AlphaMedix, RYZ101, Sn-¹¹⁷m-DTPA, TLX592, TLX66, TLX250, TLX591, TLX101, ¹²⁴I-omburtamab, GD2-SADA, ¹³¹I-omburtamab, and pharmaceutically acceptable salts thereof.

In some embodiments, the subject is suffering from, and is in need of a treatment for, a disease or condition having the symptom of cell hyperproliferation. In some embodiments, disease or condition is a cancer. In some embodiments, the cancer is a carcinoma, sarcoma, adenocarcinoma, leukemia, lymphoma, or melanoma.

In some embodiments, the cancer is a carcinoma selected from the group consisting of medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en culrasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

In some embodiments, the cancer is a sarcoma selected from the group consisting of chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

In some embodiments, the cancer is a leukemia selected from the group consisting of nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

In some embodiments, the cancer is a melanoma selected from the group consisting of acral-lentiginous melanoma, amelanotic melanoma, benign juvenile melanoma, Cloudman's melanoma, S91 melanoma, Harding-Passey melanoma, Juvenile melanoma, lentigo maligna melanoma, malignant melanoma, nodular melanoma, subungual melanoma, and superficial spreading melanoma.

In some embodiments, the cancer is prostate cancer, thyroid cancer, endocrine system cancer, brain cancer, breast cancer, cervix cancer, colon cancer, head & neck cancer, liver cancer, kidney cancer, lung cancer, non-small cell lung cancer, melanoma, mesothelioma, ovarian cancer, sarcoma, stomach cancer, uterus cancer, medulloblastoma, colorectal cancer, or pancreatic cancer.

In some embodiments, the cancer is Hodgkin's disease, Non-Hodgkin's lymphoma, multiple myeloma, neuroblastoma, glioma, glioblastoma multiforme, ovarian cancer, rhabdomyosarcoma, primary thrombocytosis, primary macroglobulinemia, primary brain tumor, malignant pancreatic insulanoma, malignant carcinoid, urinary bladder cancer, premalignant skin lesions, testicular cancer, lymphoma, thyroid cancer, esophageal cancer, genitourinary tract cancer, malignant hypercaicemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

In some embodiments, the subject is suffering from, and is in need of a treatment for, a pre-malignant condition.

### ABBREVIATIONS

Abbreviations and terms that are commonly used in the fields of organic chemistry, medicinal chemistry, pharmacology, and medicine and are well known to practitioners in these fields are used herein. Representative abbreviations and definitions are provided below:

Ac is acetyl [CH₃C(O)—], Ac₂O is acetic anhydride; AcOH is acetic acid; APC is antigen-presenting cell; aq. is aqueous; 9-BBN is 9-borabicyclo[3.3.1]nonane; BINAP is (2,2′-bis(diphenylphosphino)-1,1′-binaphthyl); Bn is benzyl; BOC is tert Butyloxycarbonyl; CDI is carbonyldiimidazole; DCM is dichloromethane; DIAD is diisopropylazodicarboxylate; DIBAL is diisobutylaluminum hydride; DIPEA is diisoproplyethyl amine; DMA is dimethylacetamide; DMAP is 4-dimethylaminopyridine; DMF is N,N-dimethylformamide; DMSO is dimethyl sulfoxide; dppf is 1,1′-bis(diphenylphosphino)ferrocene; EDAC (or EDC) is 1-ethyl-3-[3-(dimethylamino)propyl]-carbodiimide HCl; ESI is electrospray ionization mass spectrometry; Et₂O is diethyl ether; Et₃N is triethylamine; Et is ethyl; EtOAc is Ethyl acetate; EtOH is ethanol; 3-F-Ph is 3-fluorophenyl, HATU is (1-[bis(dimethylamino)methylene]-1H-1,2,3-triazolo[4,5-b]pyridinium 3-oxide hexafluorophosphate; HCl is hydrochloric acid; HOBt is 1-hydroxybenzotriazole; HPLC is high performance liquid chromatography; IPA is isopropyl alcohol; LCMS is HPLC with mass spectral detection; LiHMDS is lithium bis(trimethylsilyl)amide; LG is leaving group; M is molar; mCPBA is metachloroperbenzoic acid; mmol is millimole; Me is methyl; MeCN is acetonitrile; MeOH is methanol; Ms is methanesulfonyl; MS is mass spectrometry; MW is microwave; N is normal; NaHMDS is sodium hexamethyldisiliazide; NaOAc is sodium acetate; NaOtBu is sodium tert-butoxide; NMO is N-methylmorpholine N-oxide; NMP is N-methyl pyrrolidinone; NMR is nuclear magnetic resonance spectroscopy; Pd(PPh₃)₄ is Palladium-tetrakis(triphenylphosphine); PdCl₂(dtbpf) is [1,1′-Bis(di-tert-butylphosphino)ferrocene]dichloropalladium(II); Pd(t-Bu₃P)₂ is Bis(tri-tert-butylphosphine)palladium(0); Pd₂(dba)₃ is tris(dibenzylideneacetone)dipalladium; PdCl₂(PPh₃)₂ is dichlorobis-(triphenylphosphene) palladium; PG Denotes an unspecified protecting group; Ph is phenyl; PhMe is toluene; PPh₃ is triphenylphosphine; PMB is para-methoxybenzyl; rt is room temperature; RBF is round-bottom flask; RuPhos Pd G1 is chloro-(2-dicyclohexylphosphino-2′,6′-diisopropoxy-1,1′-biphenyl)[2-(2-aminoethyl)phenyl]palladium(II); SEM is [2-(trimethylsilyl)ethoxy]methyl; SFC is supercritical fluid chromatography; S_{N}Ar is nucleophilic aromatic substitution; S-Phos Pd G3 is (2-dicyclohexylphosphino-2′,6′-dimethoxybiphenyl) [2-(2′-amino-1,1′-biphenyl)]palladium(II) methanesulfonate: P(tBu)3 Pd G4 is 2-[2-[tert-butyl(phenyl)phosphanyl]phenyl]-1-N,1-N,3-N,3-N-tetramethylbenzene-1,3-diamine;methanesulfonic acid;N-methyl-2-phenylaniline;palladium; T3P is propanephosphonic acid anhydride; TBAB is tetrabutyl ammonium bromide; TBAF is tetrabutyl ammonium fluoride; TBS is tert-butyldimethylsilyl; tBu is tert-butyl; Tf is triflate; TFA is trifluoroacetic acid; THF is tetrahydrofuran; THP is tetrahydropyran; TLC is thin layer chromatography; TMAD is tetramethylazodicarboxamide; TMS is trimethylsilyl; TPAP is tetrapropylammonium perruthenate; Ts is p-toluenesulfonyl; UPLC is ultra performance liquid chromatography; Xantphos is (9,9-dimethyl-9H-xanthene-4,5-diyl)bis(diphenylphosphane); Xantphos Pd G3 is [(4,5-Bis(diphenylphosphino)-9,9-dimethylxanthene)-2-(2′-amino-1,1′-biphenyl)]palladium(II) methanesulfonate.

### DEFINITIONS

The term “aberrant,” as used herein, refers to different from normal. When used to describe enzymatic activity, aberrant refers to activity that is greater or less than a normal control or the average of normal non-diseased control samples. Aberrant activity may refer to an amount of activity that results in a disease, where returning the aberrant activity to a normal or non-disease-associated amount (e.g., by administering a compound or using a method as described herein), results in reduction of the disease or one or more disease symptoms. The aberrant activity can be measured by measuring the modification of a substrate of the enzyme in question; a difference of greater or equal to a 2-fold change in activity could be considered as aberrant. Aberrant activity could also refer to an increased dependence on a particular signaling pathway as a result of a deficiency in a separate complementary pathway.

The term “acyl,” as used herein, represents a group —C(═O)—R, where R is alkyl, alkenyl, alkynyl, cycloalkyl, cycloalkenyl, cycloalkynyl, aryl, heteroaryl, or heterocyclyl. Acyl may be optionally substituted as described herein for each respective R group.

The term “adenocarcinoma,” as used herein, represents a malignancy of the arising from the glandular cells that line organs within an organism. Non-limiting examples of adenocarcinomas include non-small cell lung cancer, prostate cancer, pancreatic cancer, esophageal cancer, and colorectal cancer.

The term “alkanoyl,” as used herein, represents a hydrogen or an alkyl group that is attached to the parent molecular group through a carbonyl group and is exemplified by formyl (i.e., a carboxyaldehyde group), acetyl, propionyl, butyryl, and iso-butyryl. Unsubstituted alkanoyl groups contain from 1 to 7 carbons. The alkanoyl group may be unsubstituted of substituted (e.g., optionally substituted C₁₋₇ alkanoyl) as described herein for alkyl group. The ending “-oyl” may be added to another group defined herein, e.g., aryl, cycloalkyl, and heterocyclyl, to define “aryloyl,” “cycloalkanoyl,” and “(heterocyclyl)oyl.” These groups represent a carbonyl group substituted by aryl, cycloalkyl, or heterocyclyl, respectively. Each of “aryloyl,” “cycloalkanoyl,” and “(heterocyclyl)oyl” may be optionally substituted as defined for “aryl,” “cycloalkyl,” or “heterocyclyl,” respectively.

The term “alkenyl,” as used herein, represents acyclic monovalent straight or branched chain hydrocarbon groups of containing one, two, or three carbon-carbon double bonds. Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted as defined herein for alkyl.

The term “alkenylene,” as used herein, refers to a divalent alkenyl group. An optionally substituted alkenylene is an alkenylene that is optionally substituted as described herein for alkyl.

The term “alkoxy,” as used herein, represents a chemical substituent of formula —OR, where R is a C₁₋₆ alkyl group, unless otherwise specified. In some embodiments, the alkyl group can be further substituted as defined herein. The term “alkoxy” can be combined with other terms defined herein, e.g., aryl, cycloalkyl, or heterocyclyl, to define an “aryl alkoxy,” “cycloalkyl alkoxy,” and “(heterocyclyl)alkoxy” groups. These groups represent an alkoxy that is substituted by aryl, cycloalkyl, or heterocyclyl, respectively. Each of “aryl alkoxy,” “cycloalkyl alkoxy,” and “(heterocyclyl)alkoxy” may optionally substituted as defined herein for each individual portion.

The term “alkoxyalkyl,” as used herein, represents a chemical substituent of formula -L-O—R, where L is C₁₋₆ alkylene, and R is C₁₋₆ alkyl. An optionally substituted alkoxyalkyl is an alkoxyalkyl that is optionally substituted as described herein for alkyl.

The term “alkoxycarbonylamino,” as used herein, represents a chemical substituent of formula —N(R¹)COOR², where R¹ is H or optionally substituted alkyl, and R² is optionally substituted alkyl.

The term “alkyl,” as used herein, refers to an acyclic straight or branched chain saturated hydrocarbon group, which, when unsubstituted, has from 1 to 12 carbons, unless otherwise specified. In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: amino; alkoxy; aryl; aryloxy; azido; cycloalkyl; cycloalkoxy; cycloalkenyl; cycloalkynyl; halo; heterocyclyl; (heterocyclyl)oxy; heteroaryl; hydroxy; nitro; thiol; silyl; cyano; alkylsulfonyl; alkylsulfinyl; alkylsulfenyl; ═O; ═S; —SO₂R, where R is amino or cycloalkyl; ═NR′, where R¹ is H, alkyl, aryl, or heterocyclyl. Each of the substituents may itself be unsubstituted or, valency permitting, substituted with unsubstituted substituent(s) defined herein for each respective group.

The term “alkylene,” as used herein, refers to a divalent alkyl group. An optionally substituted alkylene is an alkylene that is optionally substituted as described herein for alkyl.

The term “alkylamino,” as used herein, refers to a group having the formula —N(R^{N1})₂ or —NHR^{N1}, in which R^{N1} is alkyl, as defined herein. The alkyl portion of alkylamino can be optionally substituted as defined for alkyl. Each optional substituent on the substituted alkylamino may itself be unsubstituted or, valency permitting, substituted with unsubstituted substituent(s) defined herein for each respective group.

The term “alkylsulfenyl,” as used herein, represents a group of formula —S-(alkyl). Alkylsulfenyl may be optionally substituted as defined for alkyl.

The term “alkylsulfinyl,” as used herein, represents a group of formula —S(OHalkyl). Alkylsulfinyl may be optionally substituted as defined for alkyl.

The term “alkylsulfonyl,” as used herein, represents a group of formula —S(O)2-(alkyl). Alkylsulfonyl may be optionally substituted as defined for alkyl.

The term “alkynyl,” as used herein, represents monovalent straight or branched chain hydrocarbon groups of from two to six carbon atoms containing at least one carbon-carbon triple bond and is exemplified by ethynyl, 1-propynyl, and the like. The alkynyl groups may be unsubstituted or substituted (e.g., optionally substituted alkynyl) as defined for alkyl.

The term “allenyl,” as used herein, refers to univalent hydrocarbons having two double bonds from one carbon atom to two others, e.g., —R¹—C═C═C—R²—, where R¹ and R² are independently H, optionally substituted alkyl, optionally substituted cycloalkyl, optionally substituted aryl, or optionally substituted heterocyclyl.

The term “alkynylene,” as used herein, refers to a divalent alkynyl group. An optionally substituted alkynylene is an alkynylene that is optionally substituted as described herein for alkyl.

The term “amino,” as used herein, represents —N(R^{N1})₂, where. If amino is unsubstituted, both R^{N1} are H; or, if amino is substituted, each R^{N1} is independently H, —OH, —NO₂, —N(R^{N2})₂, —SO₂OR^{N2}, —SO₂R^{N2}, —SOR^{N2}, —COOR^{N2}, an N-protecting group, alkyl, alkenyl, alkynyl, alkoxy, aryl, arylalkyl, aryloxy, cycloalkyl, cycloalkenyl, heteroalkyl, or heterocyclyl, provided that at least one R^{N1} is not H, and where each R^{N2} Is independently H, alkyl, or aryl. Each of the substituents may itself be unsubstituted or substituted with unsubstituted substituent(s) defined herein for each respective group. In some embodiments, amino is unsubstituted amino (i.e., —NH₂) or substituted amino (e.g., NHR^{N1}), where R^{N1} is independently —OH, —SO₂OR^{N2}, —SO₂R^{N2}, —SOR^{N2}, —COOR^{N2}, optionally substituted alkyl, or optionally substituted aryl, and each R^{N2} can be optionally substituted alkyl or optionally substituted aryl. In some embodiments, substituted amino may be alkylamino, in which the alkyl groups are optionally substituted as described herein for alkyl. In some embodiments, an amino group is —NHR^{N1}, in which R^{N1} is optionally substituted alkyl.

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of: alkyl; alkenyl; alkynyl; alkoxy; alkylsulfinyl; alkylsulfenyl; alkylsulfonyl; amino; aryl; aryoxy; azido; cycloalkyl; cycloalkyl alkyl; cycloalkyl alkynyl; cycloalkoxy; cycloalkenyl; cycloalkynyl; halo; heteroalkyl; heterocyclyl; (heterocyclyl)oxy; heterocyclyl alkyl; heterocycyl alkynyl; hydroxy; nitro; thiol; silyl; and cyano. Each of the substituents may itself be unsubstituted or substituted with unsubstituted substituent(s) defined herein for each respective group.

The term “aryl alkyl,” as used herein, represents an alkyl group substituted with an aryl group.

The aryl and alkyl portions may be optionally substituted as the individual groups as described herein.

The term “arylene,” as used herein, refers to a divalent aryl group. An optionally substituted arylene is an arylene that is optionally substituted as described herein for aryl.

The term “aryloxy,” as used herein, represents a chemical substituent of formula —OR, where R is an aryl group, unless otherwise specified. In optionally substituted aryloxy, the aryl group is optionally substituted as described herein for aryl.

The term “cancer,” as used herein, refers to all types of cancer, neoplasm or malignant tumors found in mammals (e.g., humans), including leukemia, carcinomas and sarcomas. Non-limiting examples of cancers that may be treated with a compound or method provided herein include cancer of the prostate, thyroid, endocrine system, brain, breast, cervix, colon, head & neck, liver, kidney, lung, non-small cell lung, melanoma, mesothelioma, ovary, sarcoma, stomach, uterus, medulloblastoma, colorectal cancer, and pancreatic cancer. Additional non-limiting examples may include, Hodgkin's disease, Non-Hodgkin's lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulinoma, malignant carcinoid, urinary bladder cancer, premalignant skin lesions, testicular cancer, lymphoma, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, and prostate cancer.

The term “carbocyclic,” as used herein, represents an optionally substituted C₃₋₁₆ monocyclic, bicyclic, or tricyclic structure in which the rings, which may be aromatic or non-aromatic, are formed by carbon atoms. Carbocyclic structures include cycloalkyl, cycloalkenyl, cycloalkynyl, and certain aryl groups.

The term “carbonyl,” as used herein, represents a —C(O)— group.

The term “carcinoma,” as used herein, refers to a malignant new growth made up of epithelial cells tending to infiltrate the surrounding tissues and give rise to metastases. Non-limiting examples of carcinomas that may be treated with a compound or method provided herein include, e.g., medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “cyano,” as used herein, represents —CN group.

The term “cycloalkenyl,” as used herein, refers to a non-aromatic carbocyclic group having at least one double bond in the ring and from three to ten carbons (e.g., a C₃₋₁₀ cycloalkenyl), unless otherwise specified. Non-limiting examples of cycloalkenyl include cycloprop-1-enyl, cycloprop-2-enyl, cyclobut-1-enyl, cyclobut-1-enyl, cyclobut-2-enyl, cyclopent-1-enyl, cyclopent-2-enyl, cyclopent-3-enyl, norbornen-1-yl, norbornen-2-yl, norbornen-5-yl, and norbornen-7-yl. The cycloalkenyl group may be unsubstituted or substituted (e.g., optionally substituted cycloalkenyl) as described for cycloalkyl.

The term “cycloalkenyl alkyl,” as used herein, represents an alkyl group substituted with a cycloalkenyl group, each as defined herein. The cycloalkenyl and alkyl portions may be substituted as the individual groups defined herein.

The term “cycloalkoxy,” as used herein, represents a chemical substituent of formula —OR, where R is cycloalkyl group, unless otherwise specified. In some embodiments, the cycloalkyl group can be further substituted as defined herein.

The term “cycloalkyl,” as used herein, refers to a cyclic alkyl group having from three to ten carbons (e.g., a C_{3-C10} cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted (e.g., optionally substituted cycloalkyl) with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; heteroaryl; hydroxy; nitro; thiol; silyl; cyano; ═O; ═S; —SO₂R, where R is amino or cycloalkyl; ═NR′, where R′ is H, alkyl, aryl, or heterocyclyl; or —CON(R^{A})₂, where each R^{A} is independently H or alkyl, or both R^{A}, together with the atom to which they are attached, combine to form heterocycyl. Each of the substituents may itself be unsubstituted or substituted with unsubstituted substituent(s) defined herein for each respective group.

The term “cycloalkyl alkyl,” as used herein, represents an alkyl group substituted with a cycloalkyl group, each as defined herein. The cycloalkyl and alkyl portions may be optionally substituted as the individual groups described herein.

The term “cycloalkyl alkynyl,” as used herein, represents an alkynyl group substituted with a cycloalkyl group, each as defined herein. The cycloalkyl and alkynyl portions may be optionally substituted as the individual groups described herein.

The term “cycloalkylamino,” as used herein, represents a group —NHR, where R is cycloalkyl, as defined herein. An optionally substituted cycloalkylamino is a cycloalkylamino that is optionally substituted as described herein for cycloalkyl.

The term “cycloalkylene,” as used herein, represents a divalent cycloalkyl group. An optionally substituted cycloalkylene is a cycloalkylene that is optionally substituted as described herein for cycloalkyl.

The term “cycloalkynyl,” as used herein, refers to a monovalent carbocyclic group having one or two carbon-carbon triple bonds and having from eight to twelve carbons, unless otherwise specified. Cycloalkynyl may include one transannular bond or bridge. Non-limiting examples of cycloalkynyl include cyclooctynyl, cyclononynyl, cyclodecynyl, and cyclodecadiynyl. The cycloalkynyl group may be unsubstituted or substituted (e.g., optionally substituted cycloalkynyl) as defined for cycloalkyl.

The term “dicycloalkylamino,” as used herein, represents a group —NR₂, where each R is independently cycloalkyl, as defined herein. An optionally substituted dicycloalkylamino is a dicycloalkylamino that is optionally substituted as described herein for cycloalkyl.

“Disease” or “condition” refer to a state of being or health status of a patient or subject capable of being treated with the compounds or methods provided herein.

The term “halo,” as used herein, represents a halogen selected from bromine, chlorine, iodine, and fluorine.

The term “heteroalkyl,” as used herein refers to an alkyl, alkenyl, or alkynyl group interrupted once by one or two heteroatoms; twice, each time, independently, by one or two heteroatoms; three times, each time, independently, by one or two heteroatoms; or four times, each time, independently, by one or two heteroatoms. Each heteroatom is, independently, O, N, or S. In some embodiments, the heteroatom is O or N. None of the heteroalkyl groups includes two contiguous oxygen or sulfur atoms. The heteroalkyl group may be unsubstituted or substituted (e.g., optionally substituted heteroalkyl). When heteroalkyl is substituted and the substituent is bonded to the heteroatom, the substituent is selected according to the nature and valency of the heteratom. Thus, the substituent bonded to the heteroatom, valency permitting, is selected from the group consisting of ═O, —N(R^{N2})₂, —SO₂OR^{N}, —SO₂R^{N2}, —SOR^{N}, —COOR^{N}, an N protecting group, alkyl, alkenyl, alkynyl, aryl, cycloalkyl, cycloalkenyl, cycloalkynyl, heterocyclyl, or cyano, where each R^{N2} Is Independently H, alkyl, cycloalkyl, cycloalkenyl, cycloalkynyl, aryl, or heterocyclyl, and each R^{N3} is independently alkyl, cycloalkyl, cycloalkenyl, cycloalkynyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. It is understood that carbon atoms are found at the termini of a heteroalkyl group.

The term “heteroaryl alkyl,” as used herein, represents an alkyl group substituted with a heteroaryl group, each as defined herein. The heteroaryl and alkyl portions may be optionally substituted as the individual groups described herein.

The term “heteroarylene,” as used herein, represents a divalent heteroaryl. An optionally substituted heteroarylene is a heteroarylene that is optionally substituted as described herein for heteroaryl.

The term “heteroaryloxy,” as used herein, refers to a structure —OR, in which R is heteroaryl. Heteroaryloxy can be optionally substituted as defined for heterocyclyl.

The term “heterocyclyl,” as used herein, represents a monocyclic, bicyclic, tricyclic, or tetracyclic ring system having fused, bridging, and/or spiro 3-, 4-, 5-, 6-, 7-, or 8-membered rings, unless otherwise specified, containing one, two, three, or four heteroatoms independently selected from the group consisting of nitrogen, oxygen, and sulfur. In some embodiments, “heterocyclyl” is a monocyclic, bicyclic, tricyclic, or tetracyclic ring system having fused or bridging 5-, 6-, 7-, or 8-membered rings, unless otherwise specified, containing one, two, three, or four heteroatoms independently selected from the group consisting of nitrogen, oxygen, and sulfur. Sulfur may Heterocyclyl can be aromatic or non-aromatic. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups include from 1 to 16 carbon atoms unless otherwise specified. Certain heterocyclyl groups may include up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidinyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, dihydroindolyl, pyranyl, dihydropyranyl, dithiazolyl, etc. If the heterocyclic ring system has at least one aromatic resonance structure or at least one aromatic tautomer, such structure is an aromatic heterocyclyl (i.e., heteroaryl). Non-limiting examples of heteroaryl groups include benzimidazolyl, benzofuryl, benzothiazolyl, benzothienyl, benzoxazolyl, furyl, imidazolyl, indolyl, indolinyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, tetrahydroiso, quinolinyl tetrahydroquinolinyl (e.g., 1,2,3,4-tetrahydroquinolinyl), thiadiazolyl (e.g., 1,3,4-thiadiazole), thiazolyl, thienyl, triazolyl, tetrazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkyl alkyl; cycloalkyl alkynyl; cycloalkoxy; cycloalkenyl; cycloalkynyl; halo; heteroalkyl; heterocyclyl; (heterocyclyl)oxy; heterocyclyl alkyl; heterocyclyl alkynyl; hydroxy; nitro; thiol; silyl; cyano; ═O; ═S; ═NR′, where R′ Is H, alkyl, aryl, or heterocyclyl. Each of the substituents may itself be unsubstituted or substituted with unsubstituted substituent(s) defined herein for each respective group.

The term “heterocyclyl alkyl,” as used herein, represents an alkyl group substituted with a heterocyclyl group, each as defined herein. The heterocyclyl and alkyl portions may be optionally substituted as the individual groups described herein.

The term “heterocyclyl alkynyl,” as used herein, represents an alkynyl group substituted with a heterocyclyl group, each as defined herein. The heterocyclyl and alkynyl portions may be optionally substituted as the individual groups described herein.

The term “heterocyclylene,” as used herein, represents a divalent heterocyclyl. An optionally substituted heterocyclylene is a heterocyclylene that is optionally substituted as described herein for heterocyclyl.

The term “(heterocyclyl)oxy,” as used herein, represents a chemical substituent of formula —OR, where R is a heterocyclyl group, unless otherwise specified. (Heterocyclyl)oxy can be optionally substituted in a manner described for heterocyclyl.

The terms “hydroxyl” and “hydroxy,” as used interchangeably herein, represent an —OH group.

The term “isotopically enriched,” as used herein, refers to the pharmaceutically active agent with the isotopic content for one isotope at a predetermined position within a molecule that is at least 100 times greater than the natural abundance of this isotope. For example, a composition that is isotopically enriched for deuterium includes an active agent with at least one hydrogen atom position having at least 100 times greater abundance of deuterium than the natural abundance of deuterium. Preferably, an isotopic enrichment for deuterium is at least 1000 times greater than the natural abundance of deuterium. More preferably, an isotopic enrichment for deuterium is at least 4000 times greater (e.g., at least 4750 times greater, e.g., up to 5000 times greater) than the natural abundance of deuterium.

The term “leukemia,” as used herein, refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, e.g., acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphoma, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “lymphoma,” as used herein, refers to a cancer arising from cells of immune origin. Non-limiting examples of T and B cell lymphomas include non-Hodgkin lymphoma and Hodgkin disease, diffuse large B-cell lymphoma, follicular lymphoma, mucosa-associated lymphatic tissue (MALT) lymphoma, small cell lymphocytic lymphoma-chronic lymphocytic leukemia, Mantle cell lymphoma, mediastinal (thymic) large B-cell lymphoma, lymphoplasmacytic lymphoma-Waldenstrom macroglobulinemia, peripheral T-cell lymphoma (PTCL), angioimmunoblastic T-cell lymphoma (AITL)/follicular T-cell lymphoma (FTCL), anaplastic large cell lymphoma (ALCL), enteropathy-associated T-cell lymphoma (EATL), adult T-cell leukaemia/lymphoma (ATLL), or extranodal NK/T-cell lymphoma, nasal type.

The term “melanoma,” as used herein, is taken to mean a tumor arising from the melanocytic system of the skin and other organs. Melanomas that may be treated with a compound or method provided herein include, e.g., acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungual melanoma, and superficial spreading melanoma.

The term “nitro,” as used herein, represents an —NO₂ group.

The term “oxo,” as used herein, represents a divalent oxygen atom (e.g., the structure of oxo may be shown as ═O).

The term “Ph,” as used herein, represents phenyl.

The term “pharmaceutical composition,” as used herein, represents a composition containing a compound described herein, formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

The term “pharmaceutically acceptable excipient” or “pharmaceutically acceptable carrier,” as used interchangeably herein, refers to any ingredient other than the compounds described herein (e.g., a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “Polθ,” as used herein, refers to Human Polymerase theta.

The term “Polθ inhibitor,” as used herein, represents a compound that reduces the activity of Polθ in a biochemical assay, such that the measured Polθ IC₅₀ is 10 μM or less (e.g., 5 μM or less or 1 μM or less). For certain Polθ inhibitors, the Polθ IC₅₀ may be 100 nM or less (eg 10 nM or less, or 1 nM or less) and could be as low as 100 μM or 10 μM. Preferably, the Polθ IC₅₀ is 1 nM to 1 μM (e.g., 1 nM to 750 nM, 1 nM to 500 nM, or 1 nM to 250 nM).

The term “Polθ inhibitor,” as used herein, also represents a compound that upon contacting a cell expressing Polθ reduces the activity of Polθ, such that the measured Polθ IC₅₀ is 10 μM or less (e.g., 5 μM or less or 1 μM or less). For certain Polθ inhibitors, the Polθ IC₅₀ may be 100 nM or less (eg 10 nM or less, or 1 nM or less) and could be as low as 100 μM or 10 μM. Preferably, the Polθ IC₅₀ is 1 nM to 1 μM (e.g., 1 nM to 750 nM, 1 nM to 500 nM, or 1 nM to 250 nM). The term “Polθ inhibitor,” as used herein, may also represent a compound that upon contacting a cell reduces MMEJ or Alt-NHEJ activity.

The term “Polθ overexpression” refers to the increased expression or activity of Polθ in a diseases cell e.g., cancerous cell, relative to expression or activity of Polθ in a normal cell (e.g., non-diseased cell of the same kind). The amount of Polθ can be at least 2-fold, at least 3-old, at least 4-fold, at least 5-fold, at least 10-fold, or more relative to the Polθ expression in a normal cell. Examples of Polθ cancers include, but are not limited to, breast, ovarian, cervical, lung, colorectal, gastric, bladder and prostate cancers.

The term “pre-malignant” or “pre-cancerous,” as used herein, refers to a condition that is not malignant but is poised to become malignant. Non-limiting examples of pre-malignant conditions include myelodysplastic syndrome, polyps in the colon, actinic keratosis of the skin, dysplasia of the cervix, metaplasia of the lung, and leukoplakia.

The term “protecting group,” as used herein, represents a group intended to protect a hydroxy, an amino, or a carbonyl from participating in one or more undesirable reactions during chemical synthesis. The term “O-protecting group,” as used herein, represents a group intended to protect a hydroxy or carbonyl group from participating in one or more undesirable reactions during chemical synthesis. The term “N-protecting group,” as used herein, represents a group intended to protect a nitrogen containing (e.g., an amino, amido, heterocyclic N—H, or hydrazine) group from participating in one or more undesirable reactions during chemical synthesis. Commonly used O- and N-protecting groups are disclosed in Greene, “Protective Groups in Organic Synthesis,” 3rd Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkyl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p bromobenzyloxycarbonyl, 3,4-dimethoxybenzyloxycarbonyl, 3,5 dimethoxybenzyl oxycarbonyl, 2,4-dimethoxybenzyloxycarbonyl, 4 methoxybenzyloxycarbonyl, 2-nitro-4,5-dimethoxybenzyloxycarbonyl, 3,4,5 trimethoxybenzyloxycarbonyl, 1-(p-biphenylyl)-1-methylethoxycarbonyl, α,α-dimethyl-3,5 dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkyl groups such as benzyl, p-methoxybenzyl, 2,4-dimethoxybenzyl, triphenylmethyl, benzyloxymethyl, and the like, silylalkylacetal groups such as [2-(trimethylsilyl)ethoxy]methyl and silyl groups such as trimethylsilyl, and the like. Useful N-protecting groups are formyl, acetyl, benzoyl, pivaloyl, t-butylacetyl, alanyl, phenylsulfonyl, benzyl, dimethoxybenzyl, [2-(trimethylsilyl)ethoxy]methyl (SEM), tetrahydropyranyl (THP), t-butyloxycarbonyl (Boc), and benzyloxycarbonyl (Cbz).

The term “tautomer” refers to structural isomers that readily interconvert, often by relocation of a proton. Tautomers are distinct chemical species that can be identified by differing spectroscopic characteristics, but generally cannot be isolated individually. Non-limiting examples of tautomers include ketone-enol, enamine-imine, amide-imidic acid, nitroso-oxime, ketene-ynol, and amino acid-ammonium carboxylate.

The term “sarcoma” generally refers to a tumor which is made up of a substance like the embryonic connective tissue and is generally composed of closely packed cells embedded in a fibrillar or homogeneous substance. Non-limiting examples of sarcomas that may be treated with a compound or method provided herein include, e.g., a chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “subject,” as used herein, represents a human or non-human animal (e.g., a mammal) that is suffering from, or is at risk of, disease or condition, as determined by a qualified professional (e.g., a doctor or a nurse practitioner) with or without known in the art laboratory test(s) of sample(s) from the subject. Preferably, the subject is a human. Non-limiting examples of diseases and conditions include diseases having the symptom of cell hyperproliferation, e.g., a cancer.

“Treatment” and “treating,” as used herein, refer to the medical management of a subject with the intent to improve, ameliorate, stabilize, prevent or cure a disease or condition. This term includes active treatment (treatment directed to improve the disease or condition); causal treatment (treatment directed to the cause of the associated disease or condition); palliative treatment (treatment designed for the relief of symptoms of the disease or condition); preventative treatment (treatment directed to minimizing or partially or completely inhibiting the development of the associated disease or condition); and supportive treatment (treatment employed to supplement another therapy).

### DESCRIPTION OF THE DRAWINGS

FIG. 1A shows the percent viability of HCT116 BRCA2−/− cells as a function of Compound 113 concentration and olaparib concentration. FIG. 1B shows the percent viability of HCT116 BRCA2−/− cells as a function of Compound 113 concentration and niraparib concentration.

FIG. 2 shows the reduction of HCT116 BRCA2−/− tumor volume in mice treated with either vehicle. Compound 113 alone, Olaparib alone, or a combination of Compound 113 and olaparib.

FIG. 3 shows that Compound 113 sensitizes MDAMB436 breast tumor cells to AZD-7648, a DNA-PK Inhibitor.

FIG. 4 shows that Compound 113 sensitizes DLD1 BRCA2 null tumor cells to AZD-7648, a DNA-PK Inhibitor.

FIG. 5 shows that Compound 113 sensitizes DOTC24510 breast tumor cells to irradiation.

FIGS. 6A and 6B show that Compound 113 sensitizes DLD1 BRCA2 null tumors to carboplatin.

### DETAILED DESCRIPTION OF THE INVENTION

In general, the invention provides compounds, pharmaceutical compositions containing the same, methods of preparing the compounds, and methods of use. Compounds of the invention may be Polθ kinase inhibitors. The compound of the invention may be, e.g., a compound of formula (I):

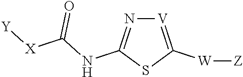

or a pharmaceutically acceptable salt thereof,
wherein
V is N or CR;
W is optionally substituted C₁₋₆ alkylene, C₁₋₆ alkoxyl, optionally substituted C₂₋₆ alkenyl, optionally substituted C₂₋₆ alkynyl, optionally substituted C₃₋₈ cycloalkyl, or optionally substituted C₆₋₁₀ aryl;
X is optionally substituted C₂₋₉ heterocyclylene, optionally substituted C₂₋₉ heteroarylene, or optionally substituted C₆₋₁₀ arylene, wherein X is further optionally substituted with -L¹-R^{X}, wherein L¹ is —O—, —NR^{X1}—, optionally substituted C₁₋₆ alkylene, optionally substituted C₁₋₆ heteroalkyl, optionally substituted C₂₋₆ alkenyl, optionally substituted allenyl, optionally substituted C₂₋₆ alkynyl, optionally substituted C₂₋₉ heterocyclylene, optionally substituted C₂₋₉ heteroarylene, or optionally substituted C₃₋₆ cycloalkylene, R^{X} is amino, halo, optionally substituted C₁₋₆ alkoxyl, optionally substituted acyl, carboxyl, amido, optionally substituted C₁₋₆ alkyl, optionally substituted C₂₋₆ heteroalkyl, optionally substituted C₂₋₉ heterocyclyl, optionally substituted C₂₋₉ heteroaryl, optionally substituted C₃₋₈ cycloalkyl C₁₋₆ alkyl, or optionally substituted C₂₋₉ heteroaryl C₁₋₆ alkyl, and R^{X1} is hydrogen or optionally substituted C₁₋₆ alkyl;
Y is optionally substituted C₂₋₆ heterocyclyl, optionally substituted C₂₋₉ heteroaryl, optionally substituted C₆₋₁₀ aryl;
Z is a H, halo, optionally substituted C₁₋₆ alkyl, optionally substituted C₂₋₆ alkynyl, optionally substituted C₁₋₆ alkoxy, optionally substituted C₂₋₉ cycloalkyl, optionally substituted C₂₋₉ heterocyclyl, optionally substituted C₂₋₉ heteroaryl, optionally substituted C₆₋₁₀ aryl, optionally substituted C₂₋₉ alkenyl, acyl, or amido; and
R is hydrogen, halogen, optionally substituted C₁₋₆ alkyl, CN, optionally substituted C₃₋₈ cycloalkyl, optionally substituted C₁₋₆ alkoxy, optionally substituted C₃₋₆ cycloalkoxy, N(R¹)₂, or C(O)NH₂, wherein each R¹ is independently hydrogen, optionally substituted C₁₋₆ alkyl, or optionally substituted C₃₋₈ cycloalkyl.

The compound of the invention may be, e.g., a compound of formula (II):

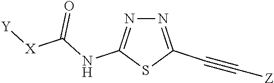

or a pharmaceutically acceptable salt thereof.

The compound of the invention may be, e.g., a compound of formula (III):

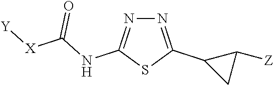

or a pharmaceutically acceptable salt thereof.

The compound of the invention may be, e.g., a compound of formula (IV):

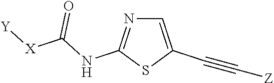

or a pharmaceutically acceptable salt thereof.

The compound of the invention may be, e.g., a compound of formula (V):

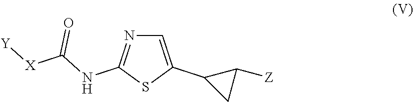

or a pharmaceutically acceptable salt thereof.

The compound of the invention may be, e.g., a compound of formula (VI):

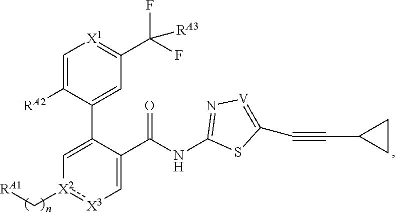

or a pharmaceutically acceptable salt thereof,
wherein
n is 0 or 1;
R^{A1} is a C₂-C₉ heteroaryl optionally substituted with C₁-C₆ alkyl or a C₄-C₉ heterocyclyl optionally substituted with oxo;
R^{A2} Is a C₁-C₆ alkyl, C₁-C₆ alkoxy, or halogen;
R^{A3} Is hydrogen or a halogen;
each of X¹ and V is independently N or CH; and
[symbol] is a single bond, X² is N, and X³ is CO, or [symbol] is a double bond, X² is C, and X³ is N or CH.

Advantageously, compounds disclosed herein may exhibit superior stability (e.g., microsomal stability) and/or superior metabolic profiles (e.g., reduced CYP3A4 Inhibition or reduced PXR activation) relative to the compounds in which the thiazole or thiadiazole core is bonded to an oxygen atom at the position proximal to the endocyclic sulfur atom.

The compound of the invention may be, e.g., a compound of formula (VII):

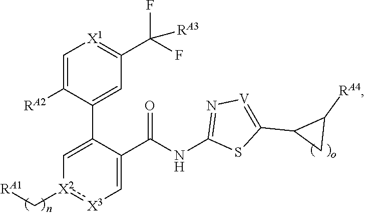

or a pharmaceutically acceptable salt thereof,
wherein
n is 0 or 1;
o is 0 or 1;
R^{A1} is a C₂-C₉ heteroaryl optionally substituted with C₁-C₆ alkyl, C₁-C₆ perfluoroalkyl, halo, or a C₄-C₉ heterocyclyl optionally substituted with oxo;
R^{A2} is a C₁-C₆ alkyl, C₁-C₆ alkoxy, or halogen;
R^{A3} is hydrogen or a halogen;
R^{A4} is optionally substituted C₁-C₆ alkyl, optionally substituted C₁-C₆ alkenyl, optionally substituted C₂₋₆ alkynyl, optionally substituted C₃-C₉ cycloalkyl, optionally substituted C₃-C₉ heterocyclylene, halo, trifluoromethyl, CN, or optionally substituted C₂₋₉ heteroarylene; or
the bond between R^{A1} and the cycloalkyl is an alkene.
[symbol] is a single bond, X² is N, and X³ is CO, or [symbol] is a double bond, X² is C, and X³ is N or CH.

In some embodiments, the compound of the invention may be, e.g., a compound listed in Table 1 below or a pharmaceutically acceptable salt thereof.

TABLE 1

| | |
|---|---|
| 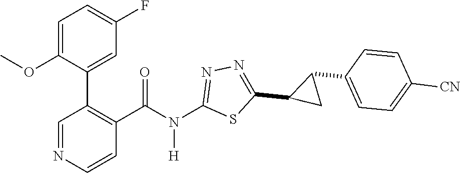 | 1 |
| 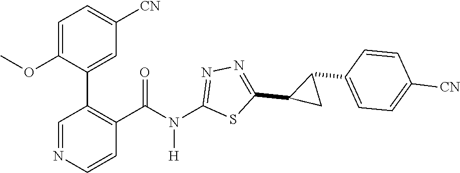 | 2 |
| 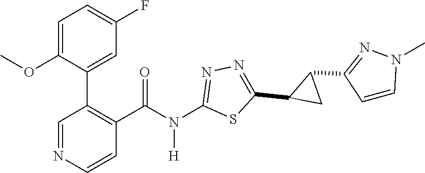 | 3 |
| 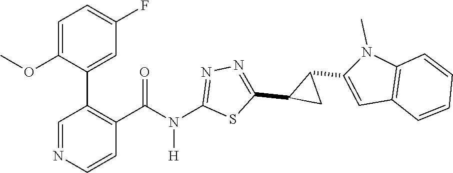 | 4 |
| 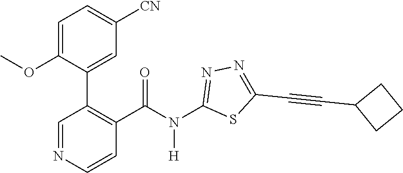 [SMILES: COc1ccc(C#N)cc1-c1cnccc1C(=O)Nc1nnc(C#CC2CCC2)s1] | 5 |
| 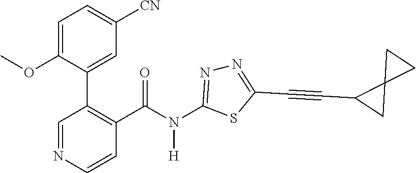 [SMILES: COc1ccc(C#N)cc1-c1cnccc1C(=O)Nc1nnc(C#CC2CC23CC3)s1] | 6 |
| 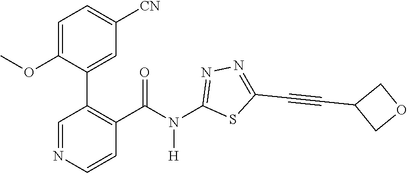 [SMILES: COc1ccc(C#N)cc1-c1cnccc1C(=O)Nc1nnc(C#CC2COC2)s1] | 7 |
| 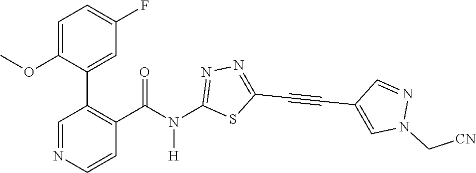 [SMILES: COc1ccc(F)cc1-c1cnccc1C(=O)Nc1nnc(C#Cc2cnn(CC#N)c2)s1] | 8 |
| 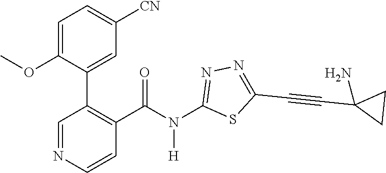 [SMILES: COc1ccc(C#N)cc1-c1cnccc1C(=O)Nc1nnc(C#CC2(N)CC2)s1] | 9 |
| 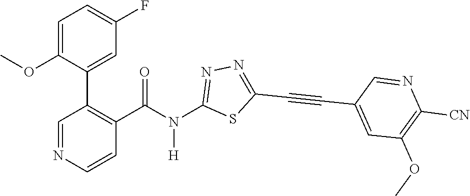 [SMILES: COc1ccc(F)cc1-c1cnccc1C(=O)Nc1nnc(C#Cc2cnc(C#N)c(OC)c2)s1] | 10 |
| 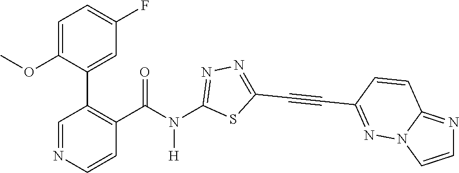 [SMILES: COc1ccc(F)cc1-c1cnccc1C(=O)Nc1nnc(C#Cc2ccc3nccn3n2)s1] | 11 |
| 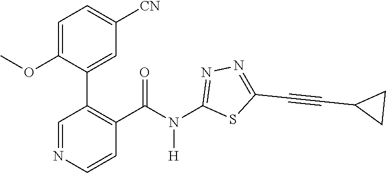 [SMILES: COc1ccc(C#N)cc1-c1cnccc1C(=O)Nc1nnc(C#CC2CC2)s1] | 12 |
| 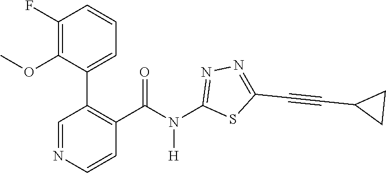 [SMILES: COc1c(F)cccc1-c1cnccc1C(=O)Nc1nnc(C#CC2CC2)s1] | 13 |
| 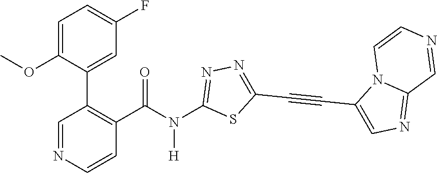 [SMILES: COc1ccc(F)cc1-c1cnccc1C(=O)Nc1nnc(C#Cc2cnc3cnccn23)s1] | 14 |
| 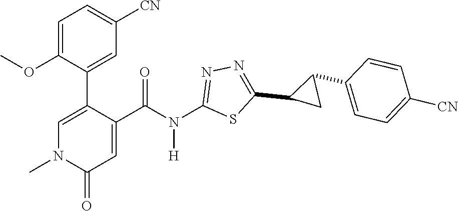 | 15 |
| 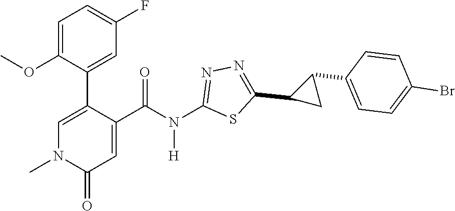 | 16 |
| 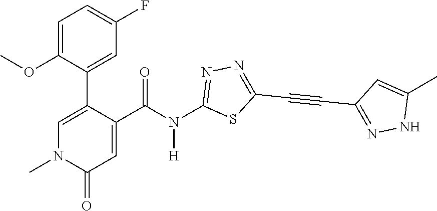 | 17 |
| 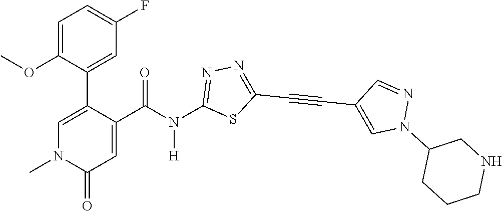 [SMILES: COc1ccc(F)cc1-c1cn(C)c(=O)cc1C(=O)Nc1nnc(C#Cc2cnn(C3CCCNC3)c2)s1] | 18 |
| 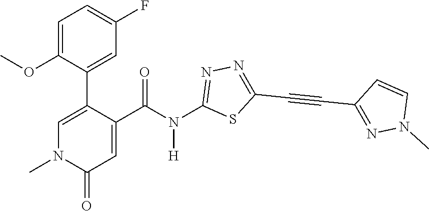 | 19 |
| 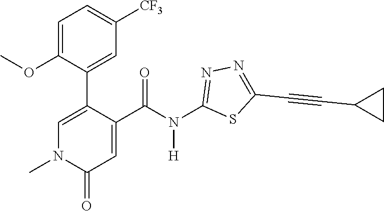 [SMILES: COc1ccc(C(F)(F)F)cc1-c1cn(C)c(=O)cc1C(=O)Nc1nnc(C#CC2CC2)s1] | 20 |
| 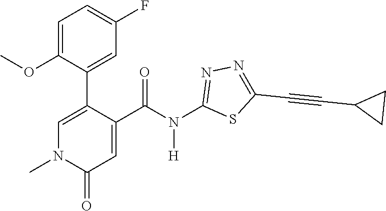 [SMILES: COc1ccc(F)cc1-c1cn(C)c(=O)cc1C(=O)Nc1nnc(C#CC2CC2)s1] | 21 |
| 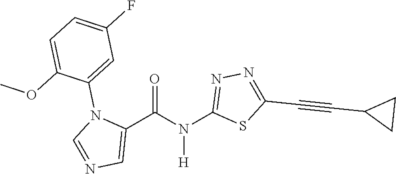 [SMILES: COc1ccc(F)cc1-n1cncc1C(=O)Nc1nnc(C#CC2CC2)s1] | 22 |
| 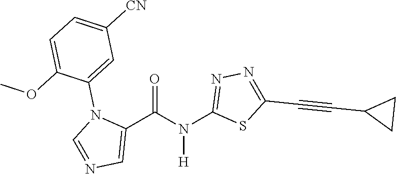 [SMILES: COc1ccc(C#N)cc1-n1cncc1C(=O)Nc1nnc(C#CC2CC2)s1] | 23 |
| 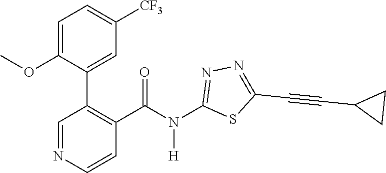 [SMILES: COc1ccc(C(F)(F)F)cc1-c1cnccc1C(=O)Nc1nnc(C#CC2CC2)s1] | 24 |
| 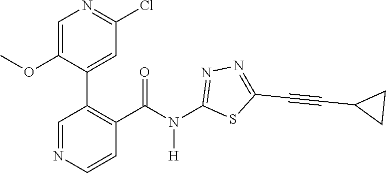 [SMILES: COc1cnc(Cl)cc1-c1cnccc1C(=O)Nc1nnc(C#CC2CC2)s1] | 25 |
| 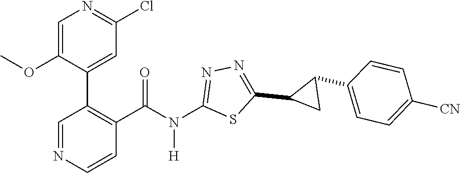 | 26 |
| 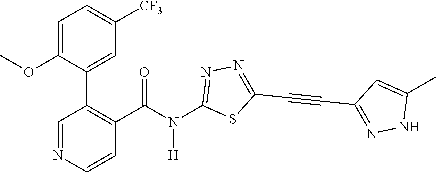 | 27 |
| 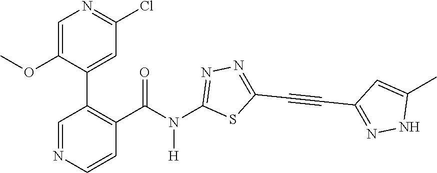 | 28 |
| 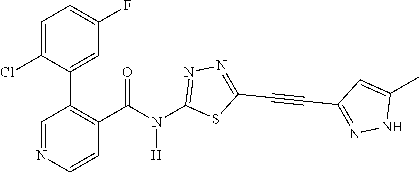 [SMILES: Cc1cc(C#Cc2nnc(NC(=O)c3ccncc3-c3cc(F)ccc3Cl)s2)n[nH]1] | 29 |
| 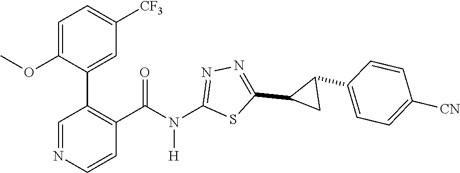 | 30 |
| 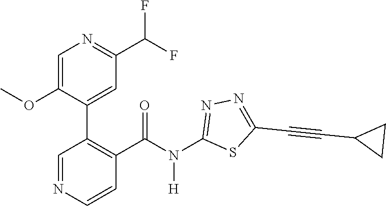 [SMILES: COc1cnc(C(F)F)cc1-c1cnccc1C(=O)Nc1nnc(C#CC2CC2)s1] | 31 |
| 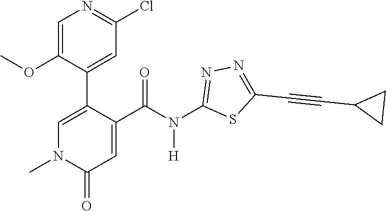 [SMILES: COc1cnc(Cl)cc1-c1cn(C)c(=O)cc1C(=O)Nc1nnc(C#CC2CC2)s1] | 32 |
| 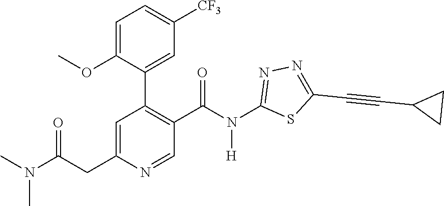 [SMILES: COc1ccc(C(F)(F)F)cc1-c1cc(CC(=O)N(C)C)ncc1C(=O)Nc1nnc(C#CC2CC2)s1] | 33 |
| 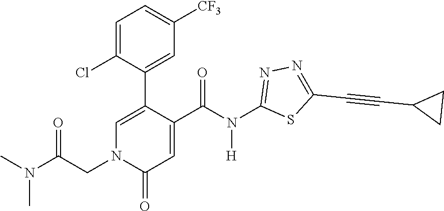 [SMILES: CN(C)C(=O)Cn1cc(-c2cc(C(F)(F)F)ccc2Cl)c(C(=O)Nc2nnc(C#CC3CC3)s2)cc1=O] | 34 |
| 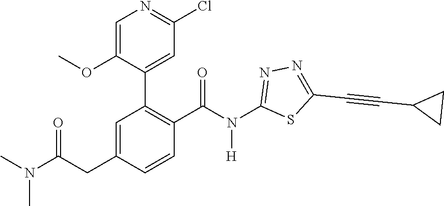 [SMILES: COc1cnc(Cl)cc1-c1cc(CC(=O)N(C)C)ccc1C(=O)Nc1nnc(C#CC2CC2)s1] | 35 |
| 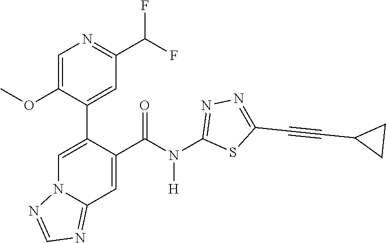 [SMILES: COc1cnc(C(F)F)cc1-c1cn2ncnc2cc1C(=O)Nc1nnc(C#CC2CC2)s1] | 36 |
| 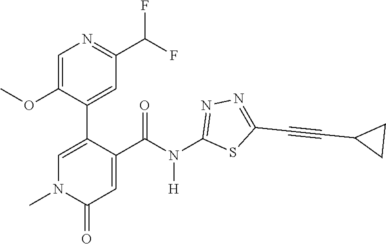 [SMILES: COc1cnc(C(F)F)cc1-c1cn(C)c(=O)cc1C(=O)Nc1nnc(C#CC2CC2)s1] | 37 |
| 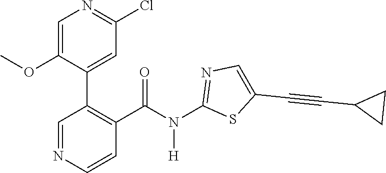 [SMILES: COc1cnc(Cl)cc1-c1cnccc1C(=O)Nc1ncc(C#CC2CC2)s1] | 38 |
| 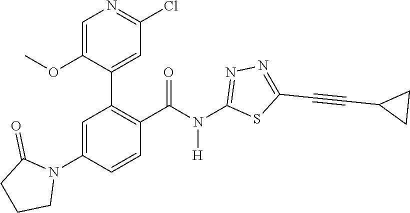 [SMILES: COc1cnc(Cl)cc1-c1cc(N2CCCC2=O)ccc1C(=O)Nc1nnc(C#CC2CC2)s1] | 39 |
| 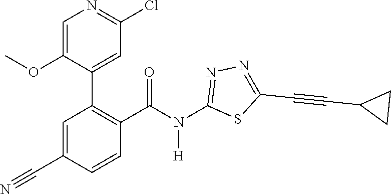 [SMILES: COc1cnc(Cl)cc1-c1cc(C#N)ccc1C(=O)Nc1nnc(C#CC2CC2)s1] | 40 |
| 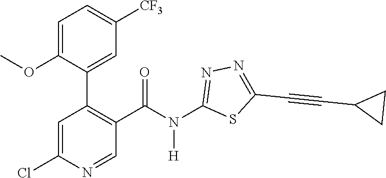 [SMILES: COc1ccc(C(F)(F)F)cc1-c1cc(Cl)ncc1C(=O)Nc1nnc(C#CC2CC2)s1] | 41 |
| 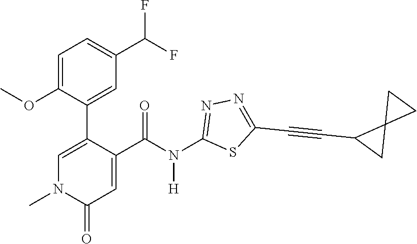 [SMILES: COc1ccc(C(F)F)cc1-c1cn(C)c(=O)cc1C(=O)Nc1nnc(C#CC2CC23CC3)s1] | 42 |
| 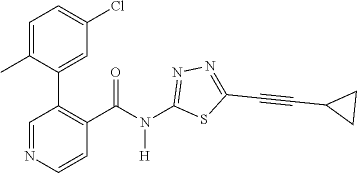 [SMILES: Cc1ccc(Cl)cc1-c1cnccc1C(=O)Nc1nnc(C#CC2CC2)s1] | 43 |
| 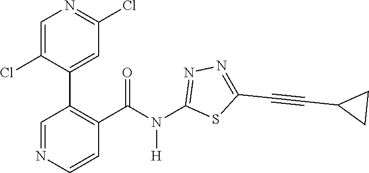 [SMILES: O=C(Nc1nnc(C#CC2CC2)s1)c1ccncc1-c1cc(Cl)ncc1Cl] | 44 |
| 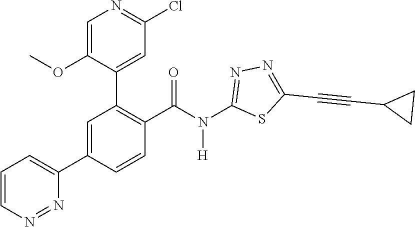 [SMILES: COc1cnc(Cl)cc1-c1cc(-c2cccnn2)ccc1C(=O)Nc1nnc(C#CC2CC2)s1] | 45 |
| 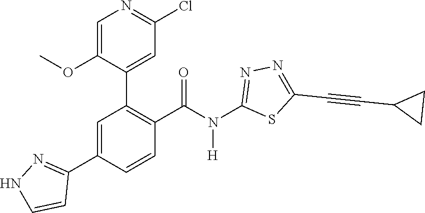 [SMILES: COc1cnc(Cl)cc1-c1cc(-c2cc[nH]n2)ccc1C(=O)Nc1nnc(C#CC2CC2)s1] | 46 |
| 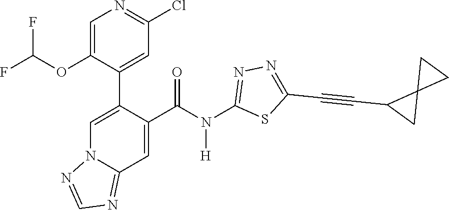 [SMILES: O=C(Nc1nnc(C#CC2CC23CC3)s1)c1cc2ncnn2cc1-c1cc(Cl)ncc1OC(F)F] | 47 |
| 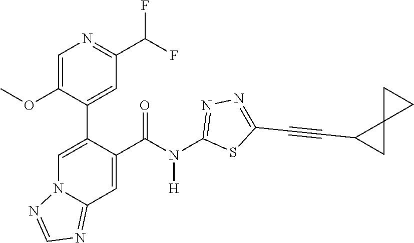 [SMILES: COc1cnc(C(F)F)cc1-c1cn2ncnc2cc1C(=O)Nc1nnc(C#CC2CC23CC3)s1] | 48 |
| 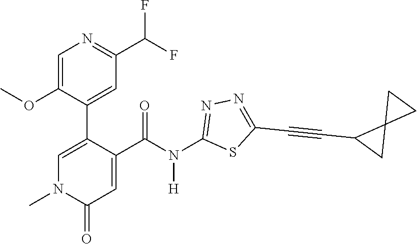 [SMILES: COc1cnc(C(F)F)cc1-c1cn(C)c(=O)cc1C(=O)Nc1nnc(C#CC2CC23CC3)s1] | 49 |
| 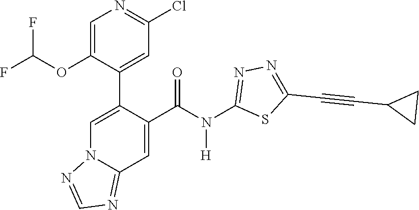 [SMILES: O=C(Nc1nnc(C#CC2CC2)s1)c1cc2ncnn2cc1-c1cc(Cl)ncc1OC(F)F] | 50 |
| 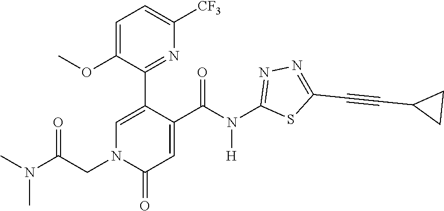 [SMILES: COc1ccc(C(F)(F)F)nc1-c1cn(CC(=O)N(C)C)c(=O)cc1C(=O)Nc1nnc(C#CC2CC2)s1] | 51 |
| 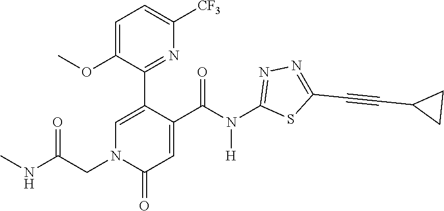 [SMILES: CNC(=O)Cn1cc(-c2nc(C(F)(F)F)ccc2OC)c(C(=O)Nc2nnc(C#CC3CC3)s2)cc1=O] | 52 |
| 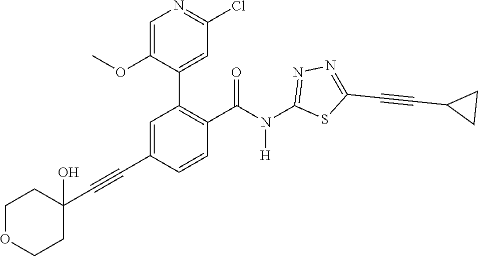 [SMILES: COc1cnc(Cl)cc1-c1cc(C#CC2(O)CCOCC2)ccc1C(=O)Nc1nnc(C#CC2CC2)s1] | 53 |
| 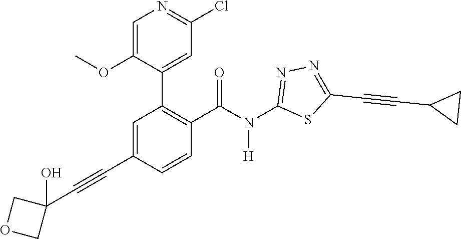 [SMILES: COc1cnc(Cl)cc1-c1cc(C#CC2(O)COC2)ccc1C(=O)Nc1nnc(C#CC2CC2)s1] | 54 |
| 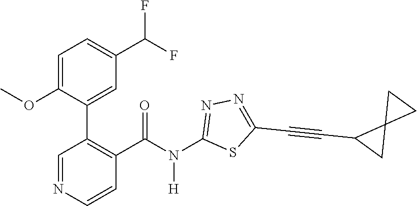 [SMILES: COc1ccc(C(F)F)cc1-c1cnccc1C(=O)Nc1nnc(C#CC2CC23CC3)s1] | 55 |
| 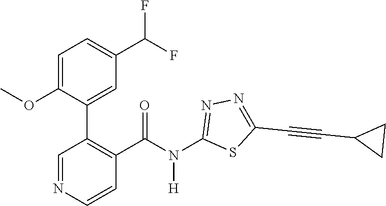 [SMILES: COc1ccc(C(F)F)cc1-c1cnccc1C(=O)Nc1nnc(C#CC2CC2)s1] | 56 |
| 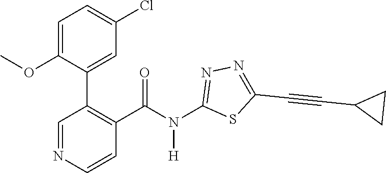 [SMILES: COc1ccc(Cl)cc1-c1cnccc1C(=O)Nc1nnc(C#CC2CC2)s1] | 57 |
| 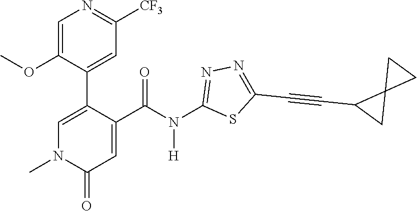 [SMILES: COc1cnc(C(F)(F)F)cc1-c1cn(C)c(=O)cc1C(=O)Nc1nnc(C#CC2CC23CC3)s1] | 58 |
| 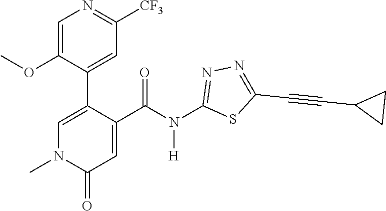 [SMILES: COc1cnc(C(F)(F)F)cc1-c1cn(C)c(=O)cc1C(=O)Nc1nnc(C#CC2CC2)s1] | 59 |
| 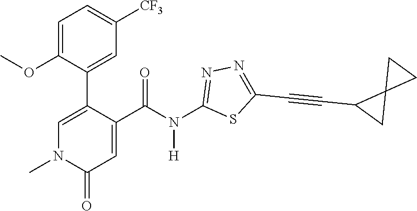 [SMILES: COc1ccc(C(F)(F)F)cc1-c1cn(C)c(=O)cc1C(=O)Nc1nnc(C#CC2CC23CC3)s1] | 60 |
| 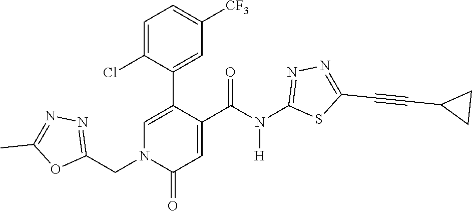 [SMILES: Cc1nnc(Cn2cc(-c3cc(C(F)(F)F)ccc3Cl)c(C(=O)Nc3nnc(C#CC4CC4)s3)cc2=O)o1] | 61 |
| 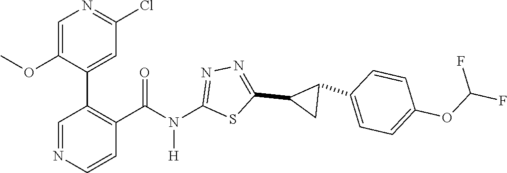 | 62 |
| 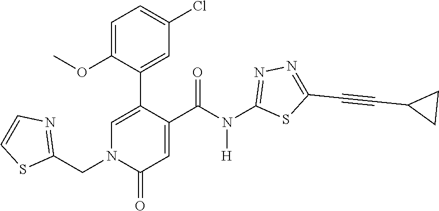 [SMILES: COc1ccc(Cl)cc1-c1cn(Cc2nccs2)c(=O)cc1C(=O)Nc1nnc(C#CC2CC2)s1] | 63 |
| 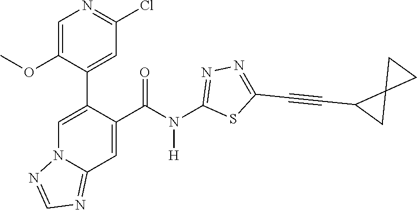 [SMILES: COc1cnc(Cl)cc1-c1cn2ncnc2cc1C(=O)Nc1nnc(C#CC2CC23CC3)s1] | 64 |
| 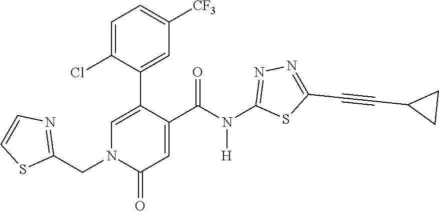 [SMILES: O=C(Nc1nnc(C#CC2CC2)s1)c1cc(=O)n(Cc2nccs2)cc1-c1cc(C(F)(F)F)ccc1Cl] | 65 |
| 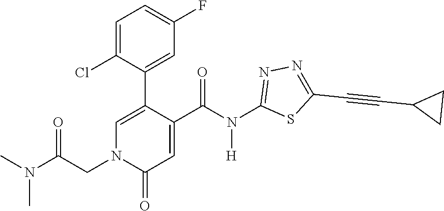 [SMILES: CN(C)C(=O)Cn1cc(-c2cc(F)ccc2Cl)c(C(=O)Nc2nnc(C#CC3CC3)s2)cc1=O] | 66 |
| 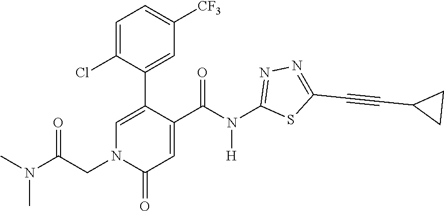 [SMILES: CN(C)C(=O)Cn1cc(-c2cc(C(F)(F)F)ccc2Cl)c(C(=O)Nc2nnc(C#CC3CC3)s2)cc1=O] | 67 |
| 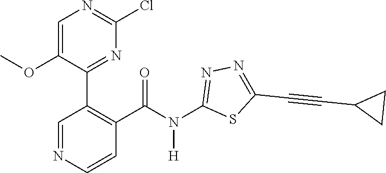 [SMILES: COc1cnc(Cl)nc1-c1cnccc1C(=O)Nc1nnc(C#CC2CC2)s1] | 68 |
| 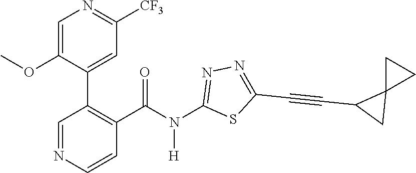 [SMILES: COc1cnc(C(F)(F)F)cc1-c1cnccc1C(=O)Nc1nnc(C#CC2CC23CC3)s1] | 69 |
| 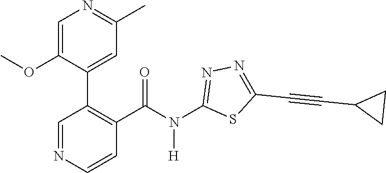 [SMILES: COc1cnc(C)cc1-c1cnccc1C(=O)Nc1nnc(C#CC2CC2)s1] | 70 |
| 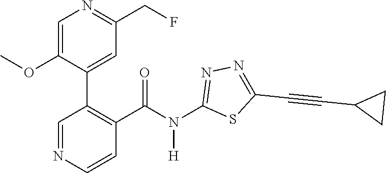 [SMILES: COc1cnc(CF)cc1-c1cnccc1C(=O)Nc1nnc(C#CC2CC2)s1] | 71 |
| 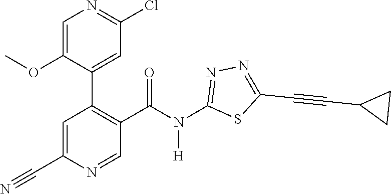 [SMILES: COc1cnc(Cl)cc1-c1cc(C#N)ncc1C(=O)Nc1nnc(C#CC2CC2)s1] | 72 |
| 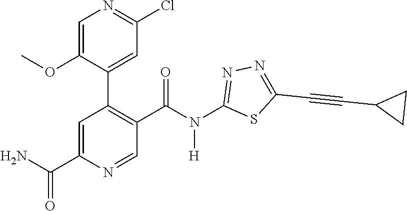 [SMILES: COc1cnc(Cl)cc1-c1cc(C(N)=O)ncc1C(=O)Nc1nnc(C#CC2CC2)s1] | 73 |
| 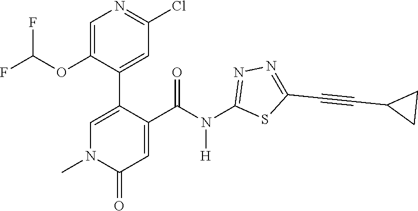 [SMILES: Cn1cc(-c2cc(Cl)ncc2OC(F)F)c(C(=O)Nc2nnc(C#CC3CC3)s2)cc1=O] | 74 |
| 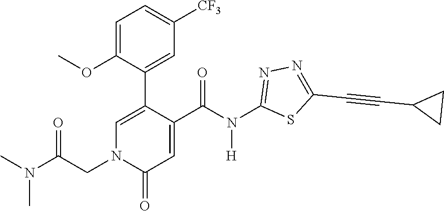 [SMILES: COc1ccc(C(F)(F)F)cc1-c1cn(CC(=O)N(C)C)c(=O)cc1C(=O)Nc1nnc(C#CC2CC2)s1] | 75 |
| 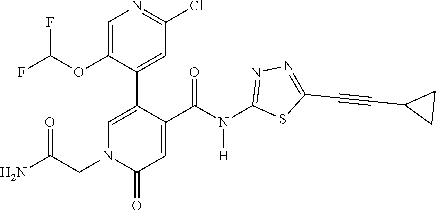 [SMILES: NC(=O)Cn1cc(-c2cc(Cl)ncc2OC(F)F)c(C(=O)Nc2nnc(C#CC3CC3)s2)cc1=O] | 76 |
| 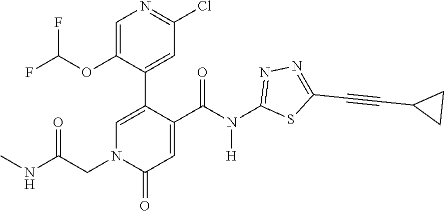 [SMILES: CNC(=O)Cn1cc(-c2cc(Cl)ncc2OC(F)F)c(C(=O)Nc2nnc(C#CC3CC3)s2)cc1=O] | 77 |
| 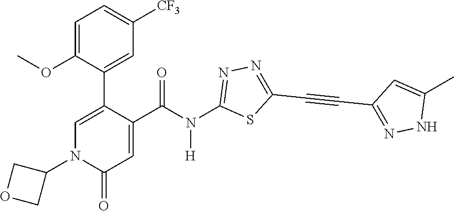 [SMILES: COc1ccc(C(F)(F)F)cc1-c1cn(C2COC2)c(=O)cc1C(=O)Nc1nnc(C#Cc2cc(C)[nH]n2)s1] | 78 |
| 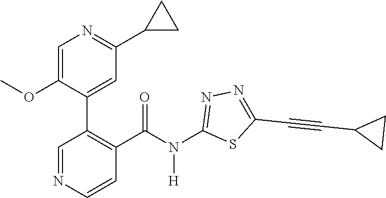 [SMILES: COc1cnc(C2CC2)cc1-c1cnccc1C(=O)Nc1nnc(C#CC2CC2)s1] | 79 |
| 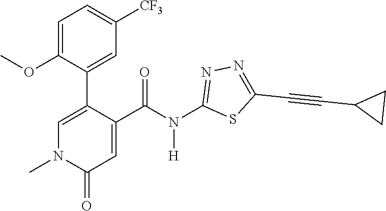 [SMILES: COc1ccc(C(F)(F)F)cc1-c1cn(C)c(=O)cc1C(=O)Nc1nnc(C#CC2CC2)s1] | 80 |
| 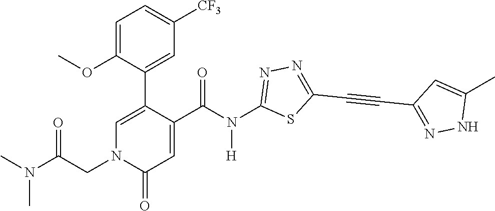 | 81 |
| 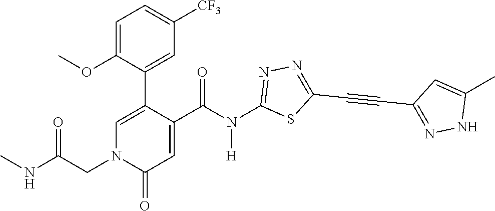 | 82 |
| 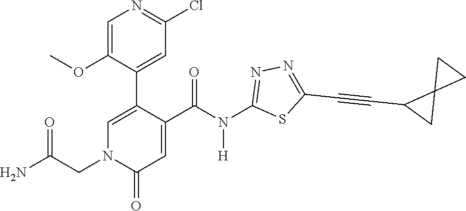 [SMILES: COc1cnc(Cl)cc1-c1cn(CC(N)=O)c(=O)cc1C(=O)Nc1nnc(C#CC2CC23CC3)s1] | 83 |
| 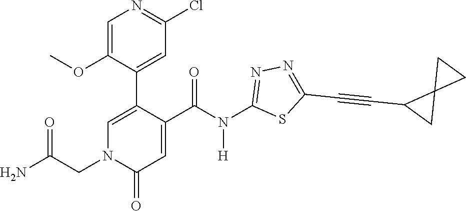 [SMILES: COc1cnc(Cl)cc1-c1cn(CC(N)=O)c(=O)cc1C(=O)Nc1nnc(C#CC2CC23CC3)s1] | 84 |
| 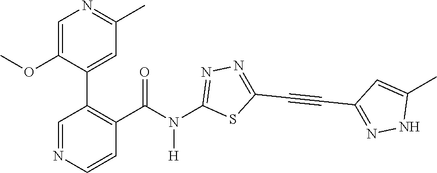 | 85 |
| 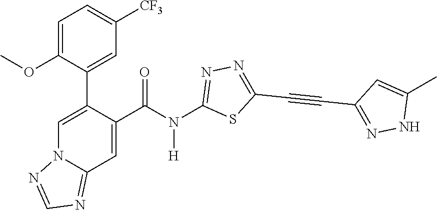 | 86 |
| 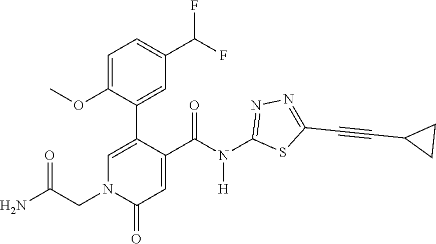 [SMILES: COc1ccc(C(F)F)cc1-c1cn(CC(N)=O)c(=O)cc1C(=O)Nc1nnc(C#CC2CC2)s1] | 87 |
| 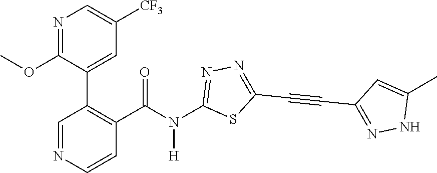 | 88 |
| 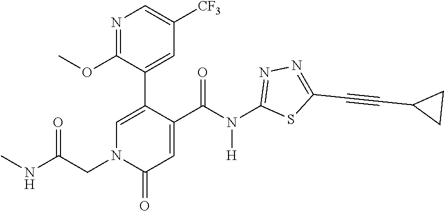 [SMILES: CNC(=O)Cn1cc(-c2cc(C(F)(F)F)cnc2OC)c(C(=O)Nc2nnc(C#CC3CC3)s2)cc1=O] | 89 |
| 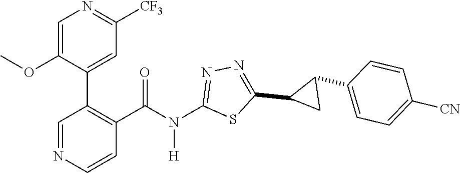 | 90 |
| 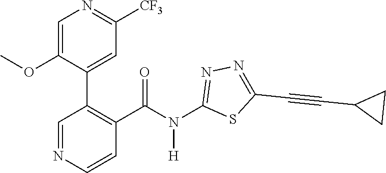 [SMILES: COc1cnc(C(F)(F)F)cc1-c1cnccc1C(=O)Nc1nnc(C#CC2CC2)s1] | 91 |
| 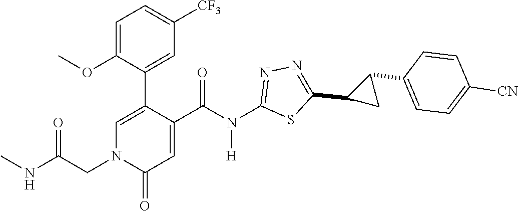 | 92 |
| 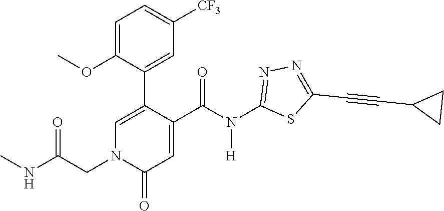 [SMILES: CNC(=O)Cn1cc(-c2cc(C(F)(F)F)ccc2OC)c(C(=O)Nc2nnc(C#CC3CC3)s2)cc1=O] | 93 |
| 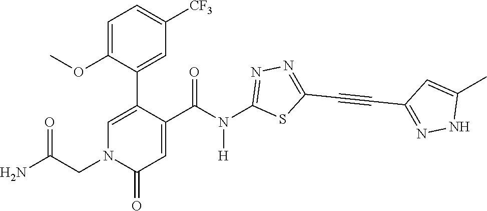 [SMILES: COc1ccc(C(F)(F)F)cc1-c1cn(CC(N)=O)c(=O)cc1C(=O)Nc1nnc(C#Cc2cc(C)[nH]n2)s1] | 94 |
| 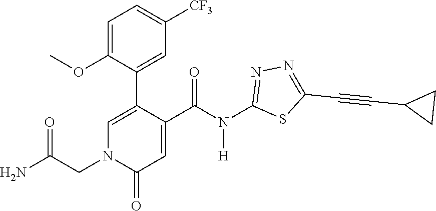 [SMILES: COc1ccc(C(F)(F)F)cc1-c1cn(CC(N)=O)c(=O)cc1C(=O)Nc1nnc(C#CC2CC2)s1] | 95 |
| 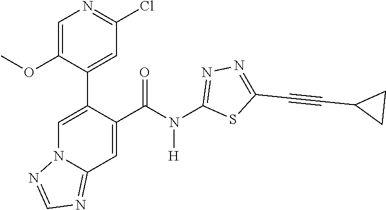 [SMILES: COc1cnc(Cl)cc1-c1cn2ncnc2cc1C(=O)Nc1nnc(C#CC2CC2)s1] | 96 |
| 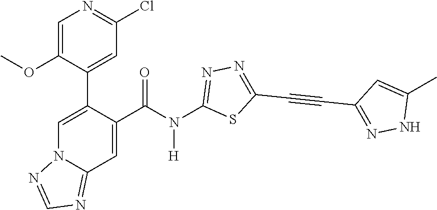 | 97 |
| 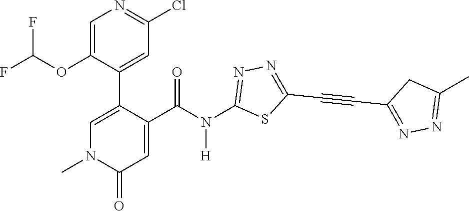 [SMILES: CC1=NN=C(C#Cc2nnc(NC(=O)c3cc(=O)n(C)cc3-c3cc(Cl)ncc3OC(F)F)s2)C1] | 98 |
| 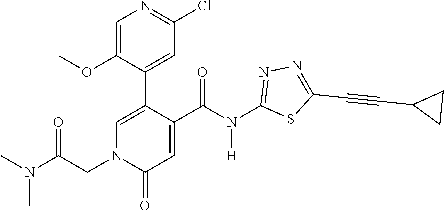 [SMILES: COc1cnc(Cl)cc1-c1cn(CC(=O)N(C)C)c(=O)cc1C(=O)Nc1nnc(C#CC2CC2)s1] | 99 |
| 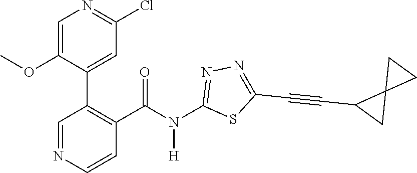 [SMILES: COc1cnc(Cl)cc1-c1cnccc1C(=O)Nc1nnc(C#CC2CC23CC3)s1] | 100 |
| 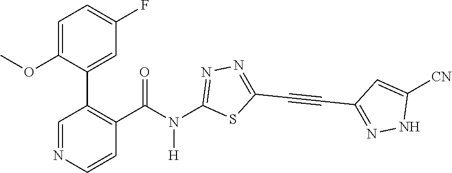 [SMILES: COc1ccc(F)cc1-c1cnccc1C(=O)Nc1nnc(C#Cc2cc(C#N)[nH]n2)s1] | 101 |
| 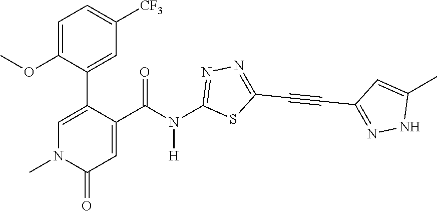 | 102 |
| 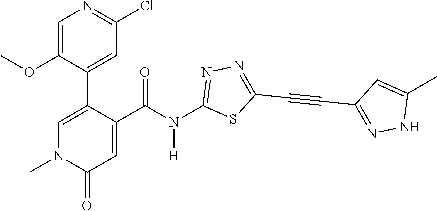 | 103 |
| 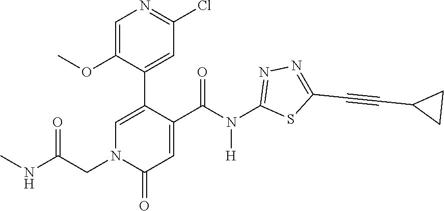 [SMILES: CNC(=O)Cn1cc(-c2cc(Cl)ncc2OC)c(C(=O)Nc2nnc(C#CC3CC3)s2)cc1=O] | 104 |
| 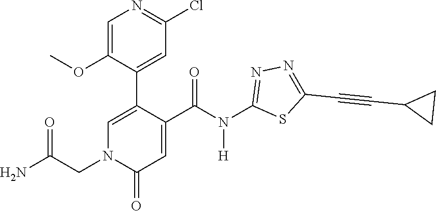 [SMILES: COc1cnc(Cl)cc1-c1cn(CC(N)=O)c(=O)cc1C(=O)Nc1nnc(C#CC2CC2)s1] | 105 |
| 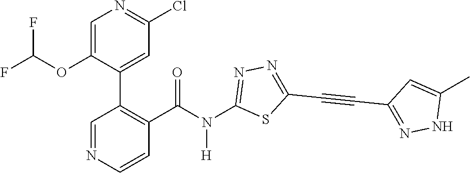 [SMILES: Cc1cc(C#Cc2nnc(NC(=O)c3ccncc3-c3cc(Cl)ncc3OC(F)F)s2)n[nH]1] | 106 |
| 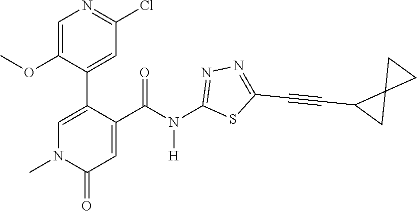 [SMILES: COc1cnc(Cl)cc1-c1cn(C)c(=O)cc1C(=O)Nc1nnc(C#CC2CC23CC3)s1] | 107 |
| 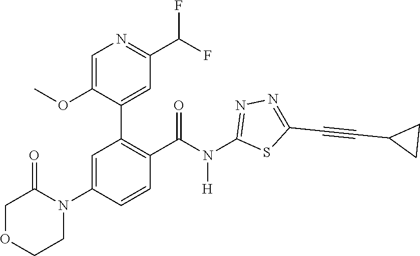 [SMILES: COc1cnc(C(F)F)cc1-c1cc(N2CCOCC2=O)ccc1C(=O)Nc1nnc(C#CC2CC2)s1] | 108 |
| 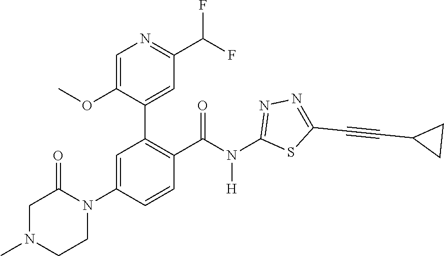 [SMILES: COc1cnc(C(F)F)cc1-c1cc(N2CCN(C)CC2=O)ccc1C(=O)Nc1nnc(C#CC2CC2)s1] | 109 |
| 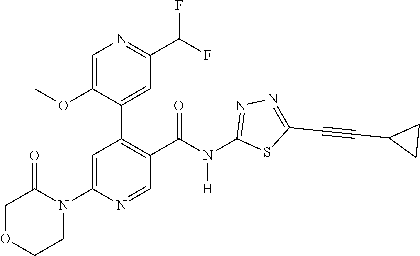 [SMILES: COc1cnc(C(F)F)cc1-c1cc(N2CCOCC2=O)ncc1C(=O)Nc1nnc(C#CC2CC2)s1] | 110 |
| 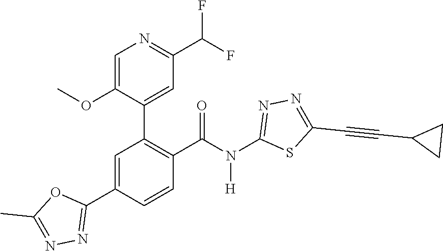 [SMILES: COc1cnc(C(F)F)cc1-c1cc(-c2nnc(C)o2)ccc1C(=O)Nc1nnc(C#CC2CC2)s1] | 111 |
| 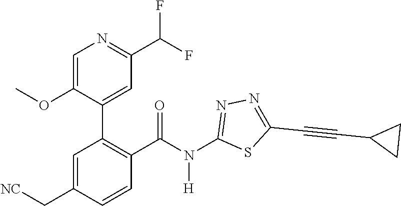 [SMILES: COc1cnc(C(F)F)cc1-c1cc(CC#N)ccc1C(=O)Nc1nnc(C#CC2CC2)s1] | 112 |
| 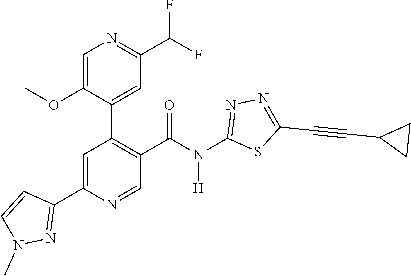 [SMILES: COc1cnc(C(F)F)cc1-c1cc(-c2ccn(C)n2)ncc1C(=O)Nc1nnc(C#CC2CC2)s1] | 113 |
| 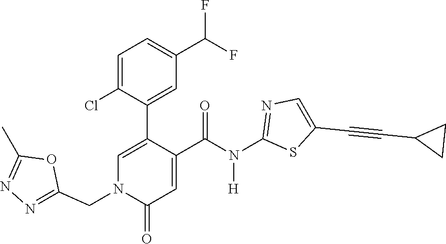 [SMILES: Cc1nnc(Cn2cc(-c3cc(C(F)F)ccc3Cl)c(C(=O)Nc3ncc(C#CC4CC4)s3)cc2=O)o1] | 114 |
| 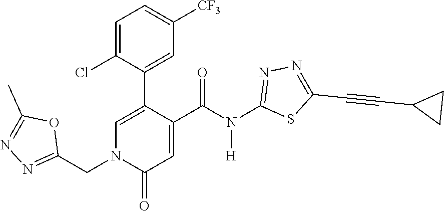 [SMILES: Cc1nnc(Cn2cc(-c3cc(C(F)(F)F)ccc3Cl)c(C(=O)Nc3nnc(C#CC4CC4)s3)cc2=O)o1] | 115 |
| 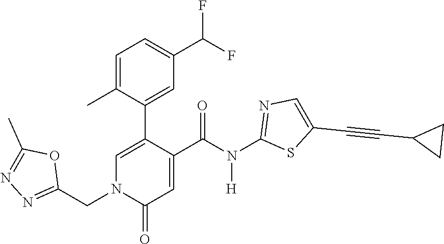 [SMILES: Cc1nnc(Cn2cc(-c3cc(C(F)F)ccc3C)c(C(=O)Nc3ncc(C#CC4CC4)s3)cc2=O)o1] | 116 |
| 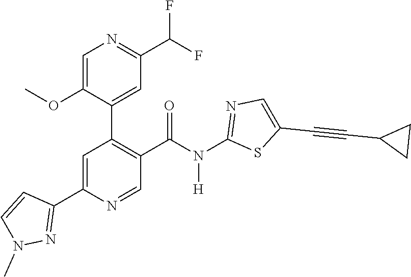 [SMILES: COc1cnc(C(F)F)cc1-c1cc(-c2ccn(C)n2)ncc1C(=O)Nc1ncc(C#CC2CC2)s1] | 117 |
| 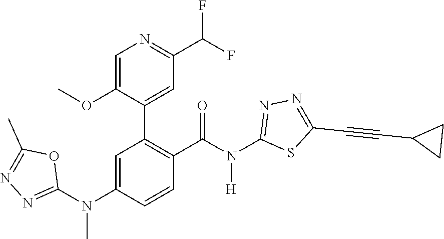 [SMILES: COc1cnc(C(F)F)cc1-c1cc(N(C)c2nnc(C)o2)ccc1C(=O)Nc1nnc(C#CC2CC2)s1] | 118 |
| 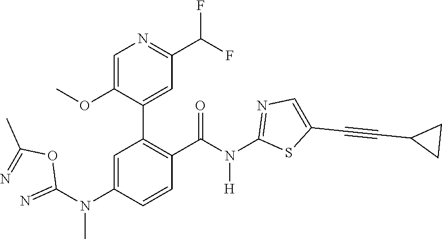 [SMILES: COc1cnc(C(F)F)cc1-c1cc(N(C)C(=N)OC(C)=N)ccc1C(=O)Nc1ncc(C#CC2CC2)s1] | 119 |
| 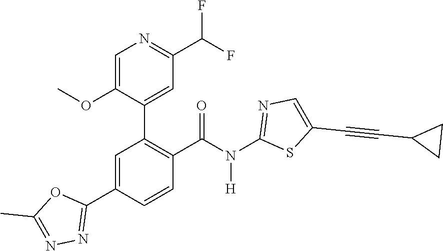 [SMILES: COc1cnc(C(F)F)cc1-c1cc(-c2nnc(C)o2)ccc1C(=O)Nc1ncc(C#CC2CC2)s1] | 120 |
| 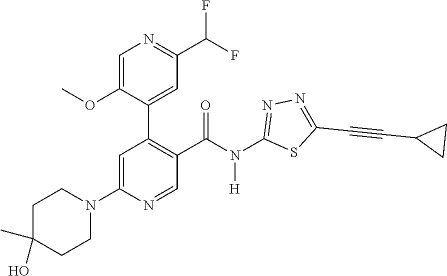 [SMILES: COc1cnc(C(F)F)cc1-c1cc(N2CCC(C)(O)CC2)ncc1C(=O)Nc1nnc(C#CC2CC2)s1] | 121 |
| 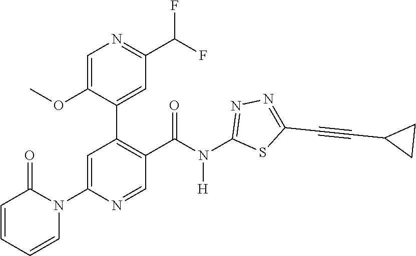 [SMILES: COc1cnc(C(F)F)cc1-c1cc(-n2ccccc2=O)ncc1C(=O)Nc1nnc(C#CC2CC2)s1] | 122 |
| 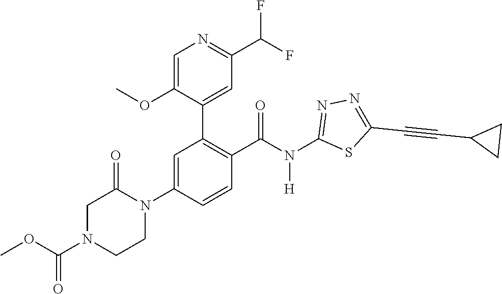 [SMILES: COC(=O)N1CCN(c2ccc(C(=O)Nc3nnc(C#CC4CC4)s3)c(-c3cc(C(F)F)ncc3OC)c2)C(=O)C1] | 123 |
| 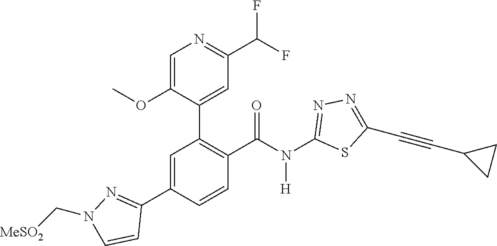 | 124 |
| 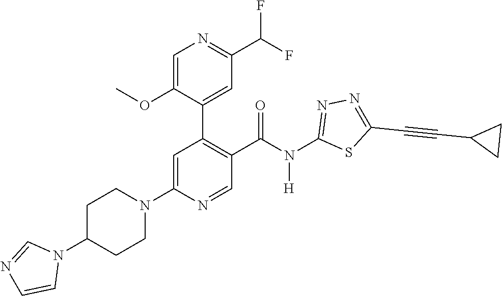 [SMILES: COc1cnc(C(F)F)cc1-c1cc(N2CCC(n3ccnc3)CC2)ncc1C(=O)Nc1nnc(C#CC2CC2)s1] | 125 |
| 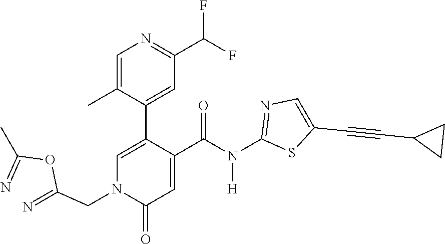 [SMILES: CC(=N)OC(=N)Cn1cc(-c2cc(C(F)F)ncc2C)c(C(=O)Nc2ncc(C#CC3CC3)s2)cc1=O] | 126 |
| 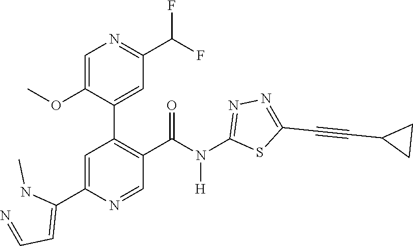 [SMILES: CN/C(=C\C=N)c1cc(-c2cc(C(F)F)ncc2OC)c(C(=O)Nc2nnc(C#CC3CC3)s2)cn1] | 127 |
| 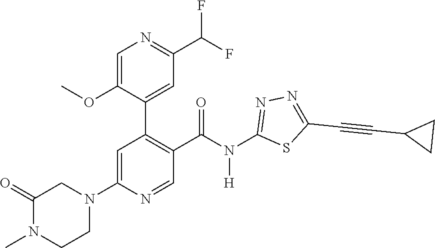 [SMILES: COc1cnc(C(F)F)cc1-c1cc(N2CCN(C)C(=O)C2)ncc1C(=O)Nc1nnc(C#CC2CC2)s1] | 128 |
| 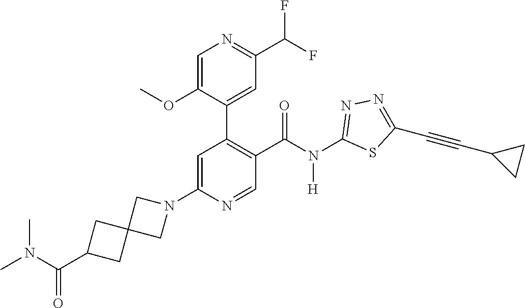 [SMILES: COc1cnc(C(F)F)cc1-c1cc(N2CC3(CC(C(=O)N(C)C)C3)C2)ncc1C(=O)Nc1nnc(C#CC2CC2)s1] | 129 |
| 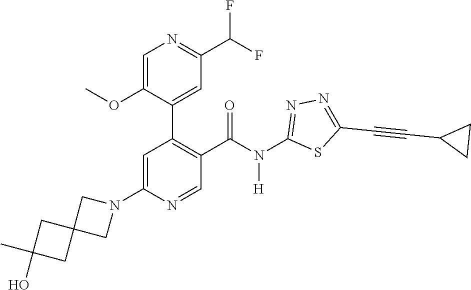 [SMILES: COc1cnc(C(F)F)cc1-c1cc(N2CC3(C2)CC(C)(O)C3)ncc1C(=O)Nc1nnc(C#CC2CC2)s1] | 130 |
| 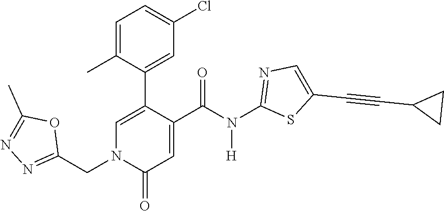 [SMILES: Cc1nnc(Cn2cc(-c3cc(Cl)ccc3C)c(C(=O)Nc3ncc(C#CC4CC4)s3)cc2=O)o1] | 131 |
| 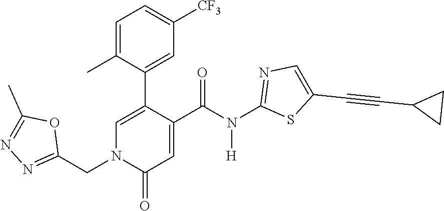 [SMILES: Cc1nnc(Cn2cc(-c3cc(C(F)(F)F)ccc3C)c(C(=O)Nc3ncc(C#CC4CC4)s3)cc2=O)o1] | 132 |
| 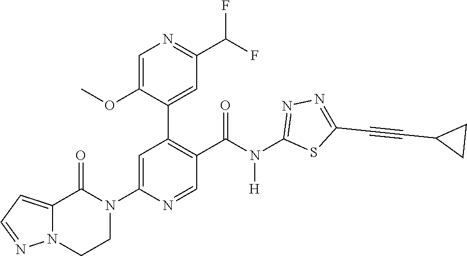 [SMILES: COc1cnc(C(F)F)cc1-c1cc(N2CCn3nccc3C2=O)ncc1C(=O)Nc1nnc(C#CC2CC2)s1] | 133 |
| 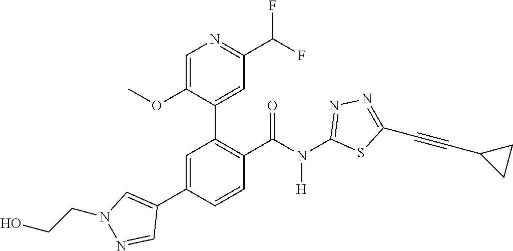 [SMILES: COc1cnc(C(F)F)cc1-c1cc(-c2cnn(CCO)c2)ccc1C(=O)Nc1nnc(C#CC2CC2)s1] | 134 |
| 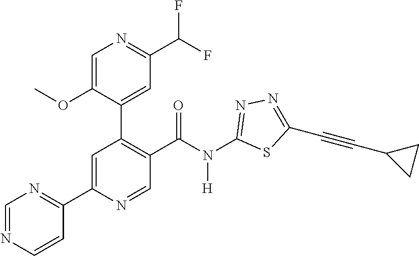 [SMILES: COc1cnc(C(F)F)cc1-c1cc(-c2ccncn2)ncc1C(=O)Nc1nnc(C#CC2CC2)s1] | 135 |
| 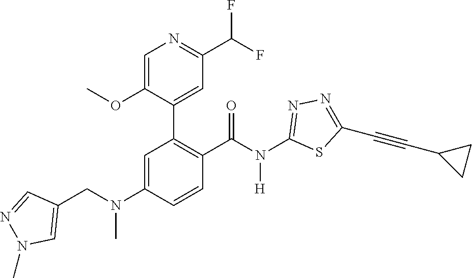 [SMILES: COc1cnc(C(F)F)cc1-c1cc(N(C)Cc2cnn(C)c2)ccc1C(=O)Nc1nnc(C#CC2CC2)s1] | 136 |
| 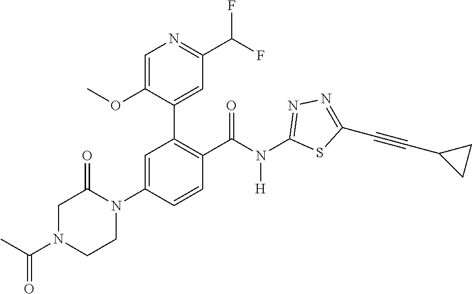 [SMILES: COc1cnc(C(F)F)cc1-c1cc(N2CCN(C(C)=O)CC2=O)ccc1C(=O)Nc1nnc(C#CC2CC2)s1] | 137 |
| 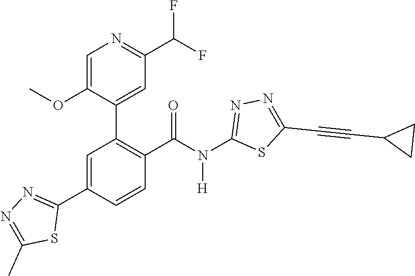 [SMILES: COc1cnc(C(F)F)cc1-c1cc(-c2nnc(C)s2)ccc1C(=O)Nc1nnc(C#CC2CC2)s1] | 138 |
| 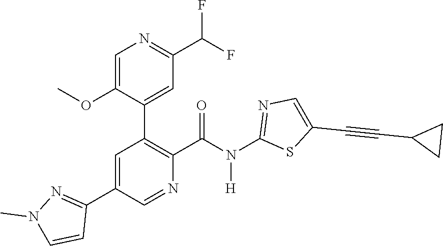 [SMILES: COc1cnc(C(F)F)cc1-c1cc(-c2ccn(C)n2)cnc1C(=O)Nc1ncc(C#CC2CC2)s1] | 139 |
| 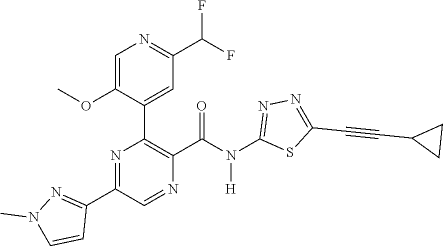 [SMILES: COc1cnc(C(F)F)cc1-c1nc(-c2ccn(C)n2)cnc1C(=O)Nc1nnc(C#CC2CC2)s1] | 140 |
| 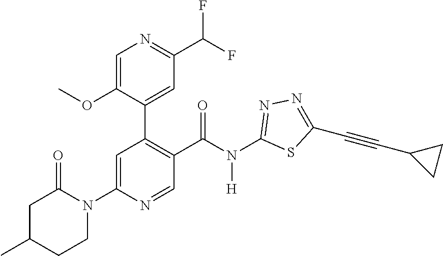 [SMILES: COc1cnc(C(F)F)cc1-c1cc(N2CCC(C)CC2=O)ncc1C(=O)Nc1nnc(C#CC2CC2)s1] | 141 |
| 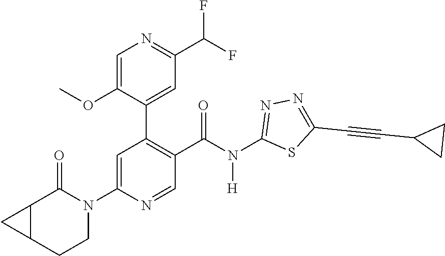 [SMILES: COc1cnc(C(F)F)cc1-c1cc(N2CCC3CC3C2=O)ncc1C(=O)Nc1nnc(C#CC2CC2)s1] | 142 |
| 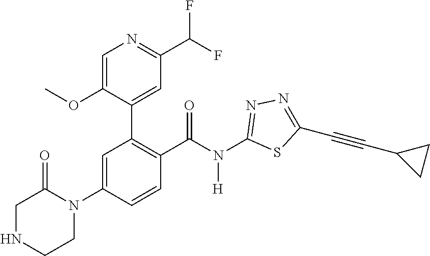 [SMILES: COc1cnc(C(F)F)cc1-c1cc(N2CCNCC2=O)ccc1C(=O)Nc1nnc(C#CC2CC2)s1] | 143 |
| 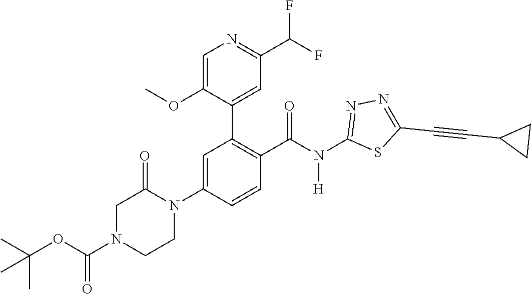 [SMILES: COc1cnc(C(F)F)cc1-c1cc(N2CCN(C(=O)OC(C)(C)C)CC2=O)ccc1C(=O)Nc1nnc(C#CC2CC2)s1] | 144 |
| 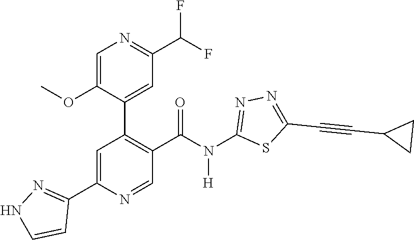 [SMILES: COc1cnc(C(F)F)cc1-c1cc(-c2cc[nH]n2)ncc1C(=O)Nc1nnc(C#CC2CC2)s1] | 145 |
| 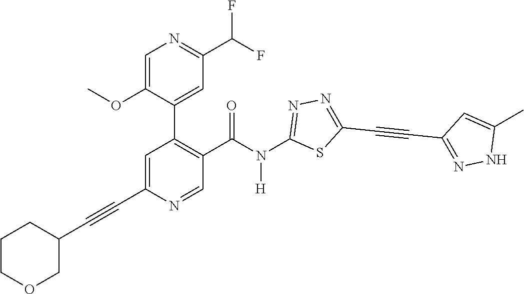 [SMILES: COc1cnc(C(F)F)cc1-c1cc(C#CC2CCCOC2)ncc1C(=O)Nc1nnc(C#Cc2cc(C)[nH]n2)s1] | 146 |
| 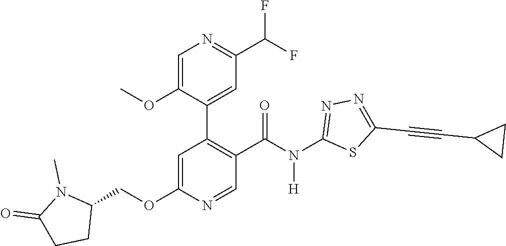 [SMILES: COc1cnc(C(F)F)cc1-c1cc(OC[C@@H]2CCC(=O)N2C)ncc1C(=O)Nc1nnc(C#CC2CC2)s1] | 147 |
| 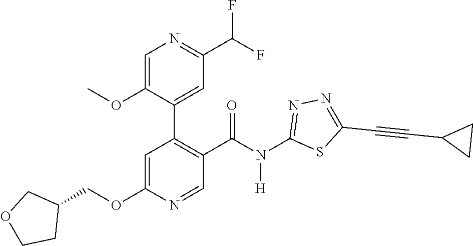 [SMILES: COc1cnc(C(F)F)cc1-c1cc(OC[C@@H]2CCOC2)ncc1C(=O)Nc1nnc(C#CC2CC2)s1] | 148 |
| 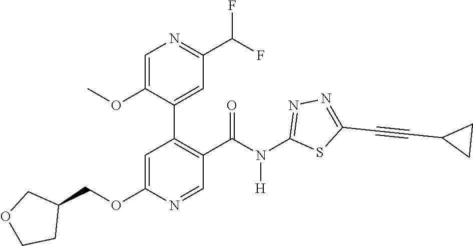 [SMILES: COc1cnc(C(F)F)cc1-c1cc(OC[C@H]2CCOC2)ncc1C(=O)Nc1nnc(C#CC2CC2)s1] | 149 |
| 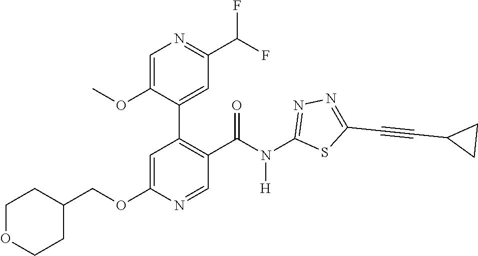 [SMILES: COc1cnc(C(F)F)cc1-c1cc(OCC2CCOCC2)ncc1C(=O)Nc1nnc(C#CC2CC2)s1] | 150 |
| 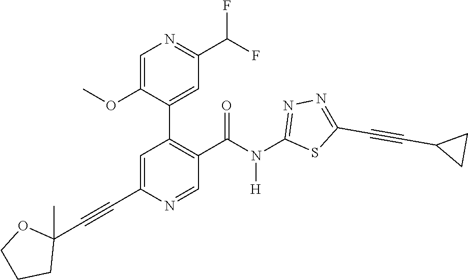 [SMILES: COc1cnc(C(F)F)cc1-c1cc(C#CC2(C)CCCO2)ncc1C(=O)Nc1nnc(C#CC2CC2)s1] | 151 |
| 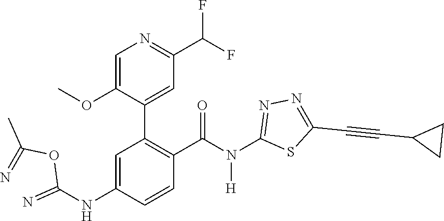 [SMILES: COc1cnc(C(F)F)cc1-c1cc(NC(=N)OC(C)=N)ccc1C(=O)Nc1nnc(C#CC2CC2)s1] | 152 |
| 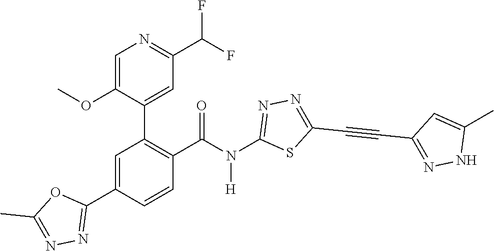 | 153 |
| 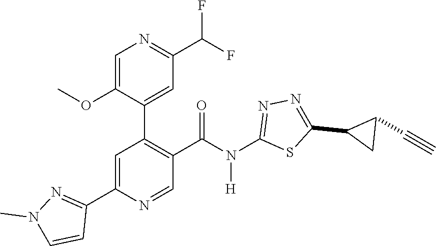 | 154 |
| 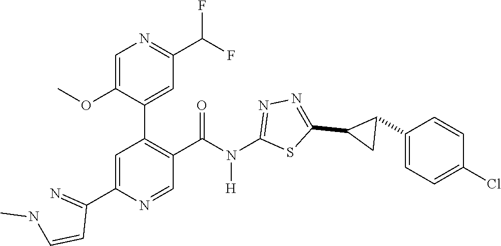 | 155 |
| 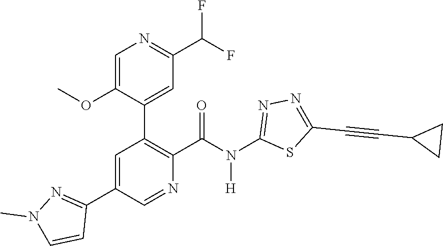 [SMILES: COc1cnc(C(F)F)cc1-c1cc(-c2ccn(C)n2)cnc1C(=O)Nc1nnc(C#CC2CC2)s1] | 156 |
| 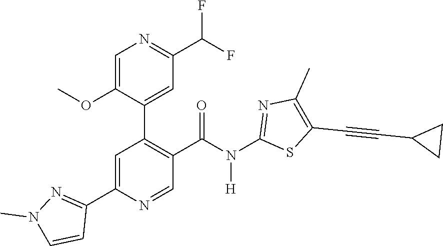 [SMILES: COc1cnc(C(F)F)cc1-c1cc(-c2ccn(C)n2)ncc1C(=O)Nc1nc(C)c(C#CC2CC2)s1] | 157 |
| 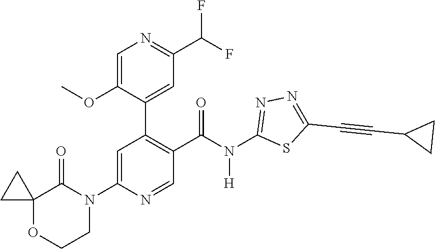 [SMILES: COc1cnc(C(F)F)cc1-c1cc(N2CCOC3(CC3)C2=O)ncc1C(=O)Nc1nnc(C#CC2CC2)s1] | 158 |
| 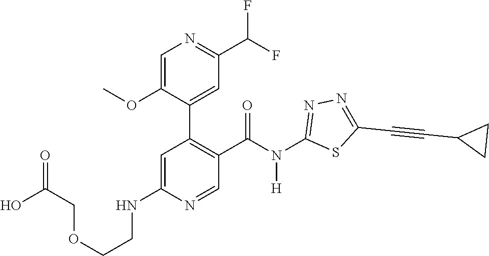 [SMILES: COc1cnc(C(F)F)cc1-c1cc(NCCOCC(=O)O)ncc1C(=O)Nc1nnc(C#CC2CC2)s1] | 159 |
| 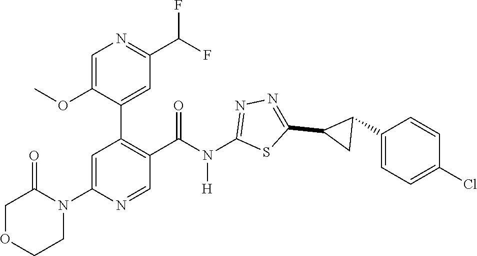 | 160 |
| 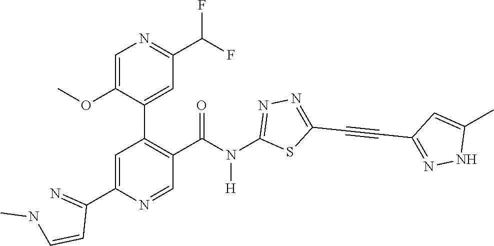 | 161 |
| 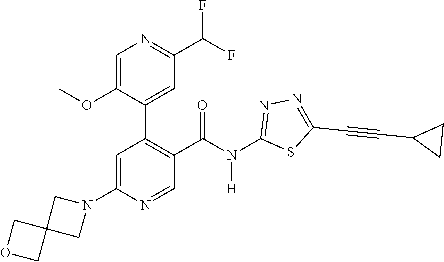 [SMILES: COc1cnc(C(F)F)cc1-c1cc(N2CC3(COC3)C2)ncc1C(=O)Nc1nnc(C#CC2CC2)s1] | 162 |
| 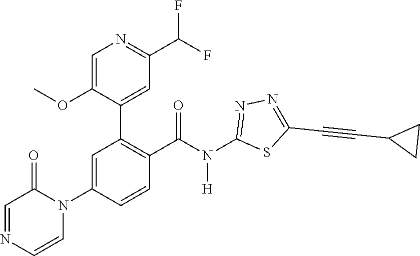 [SMILES: COc1cnc(C(F)F)cc1-c1cc(-n2ccncc2=O)ccc1C(=O)Nc1nnc(C#CC2CC2)s1] | 163 |
| 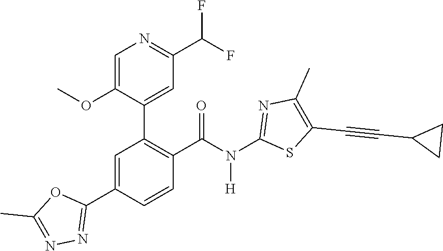 [SMILES: COc1cnc(C(F)F)cc1-c1cc(-c2nnc(C)o2)ccc1C(=O)Nc1nc(C)c(C#CC2CC2)s1] | 164 |
| 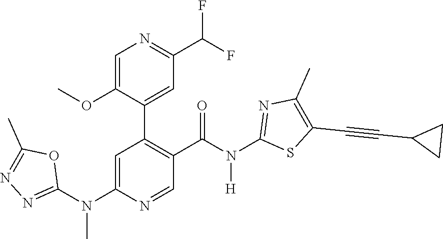 [SMILES: COc1cnc(C(F)F)cc1-c1cc(N(C)c2nnc(C)o2)ncc1C(=O)Nc1nc(C)c(C#CC2CC2)s1] | 165 |
| 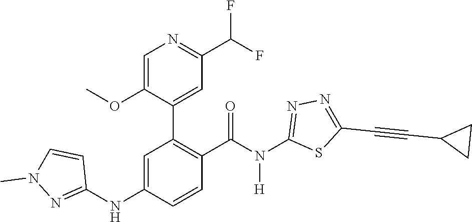 [SMILES: COc1cnc(C(F)F)cc1-c1cc(Nc2ccn(C)n2)ccc1C(=O)Nc1nnc(C#CC2CC2)s1] | 166 |
| 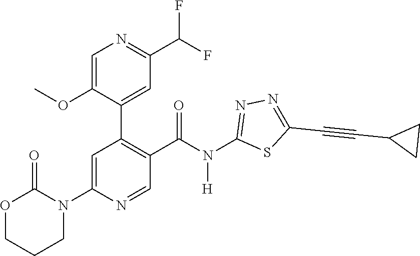 [SMILES: COc1cnc(C(F)F)cc1-c1cc(N2CCCOC2=O)ncc1C(=O)Nc1nnc(C#CC2CC2)s1] | 167 |
| 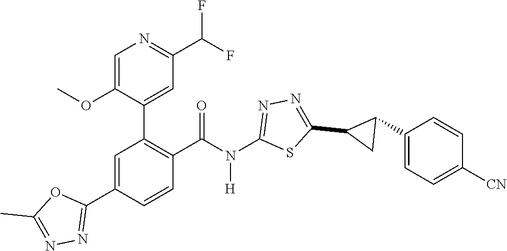 | 168 |
| 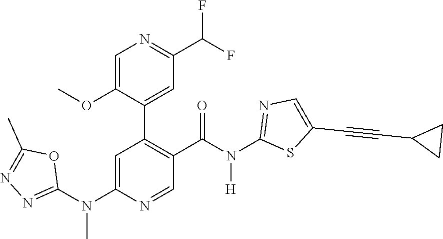 [SMILES: COc1cnc(C(F)F)cc1-c1cc(N(C)c2nnc(C)o2)ncc1C(=O)Nc1ncc(C#CC2CC2)s1] | 169 |
| 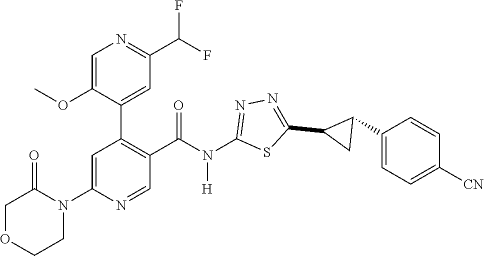 | 170 |
| 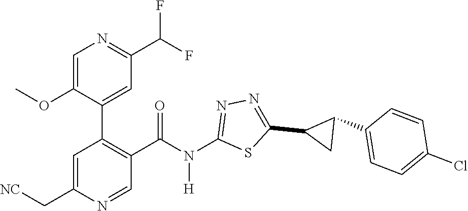 | 171 |
| 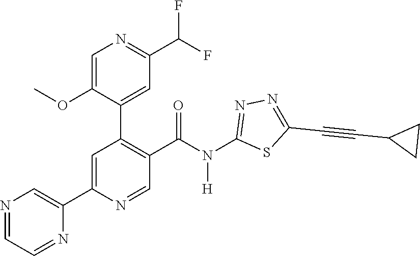 [SMILES: COc1cnc(C(F)F)cc1-c1cc(-c2cnccn2)ncc1C(=O)Nc1nnc(C#CC2CC2)s1] | 172 |
| 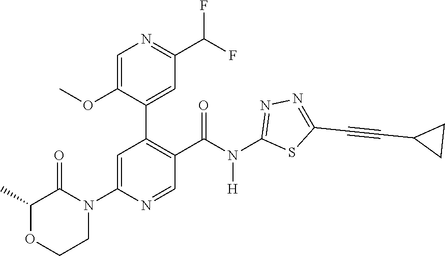 [SMILES: COc1cnc(C(F)F)cc1-c1cc(N2CCO[C@H](C)C2=O)ncc1C(=O)Nc1nnc(C#CC2CC2)s1] | 173 |
| 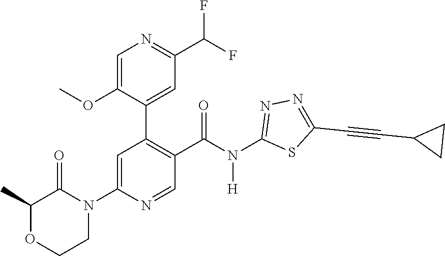 [SMILES: COc1cnc(C(F)F)cc1-c1cc(N2CCO[C@@H](C)C2=O)ncc1C(=O)Nc1nnc(C#CC2CC2)s1] | 174 |
| 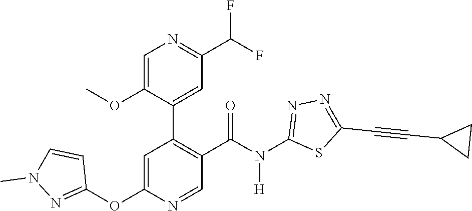 [SMILES: COc1cnc(C(F)F)cc1-c1cc(Oc2ccn(C)n2)ncc1C(=O)Nc1nnc(C#CC2CC2)s1] | 175 |
| 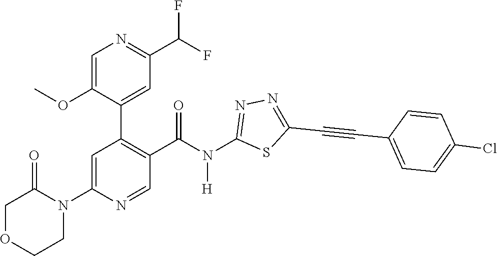 [SMILES: COc1cnc(C(F)F)cc1-c1cc(N2CCOCC2=O)ncc1C(=O)Nc1nnc(C#Cc2ccc(Cl)cc2)s1] | 176 |
| 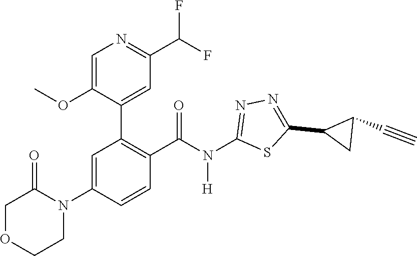 | 177 |
| 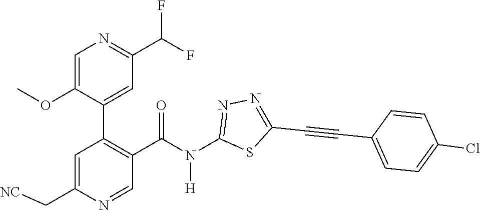 [SMILES: COc1cnc(C(F)F)cc1-c1cc(CC#N)ncc1C(=O)Nc1nnc(C#Cc2ccc(Cl)cc2)s1] | 178 |
| 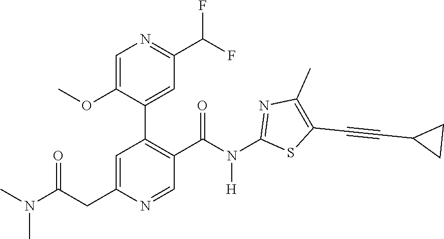 [SMILES: COc1cnc(C(F)F)cc1-c1cc(CC(=O)N(C)C)ncc1C(=O)Nc1nc(C)c(C#CC2CC2)s1] | 179 |
| 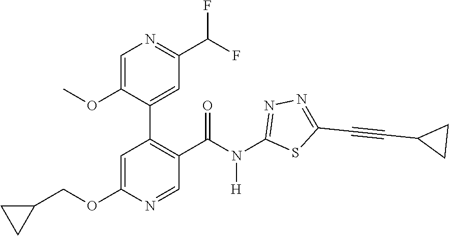 [SMILES: COc1cnc(C(F)F)cc1-c1cc(OCC2CC2)ncc1C(=O)Nc1nnc(C#CC2CC2)s1] | 180 |
| 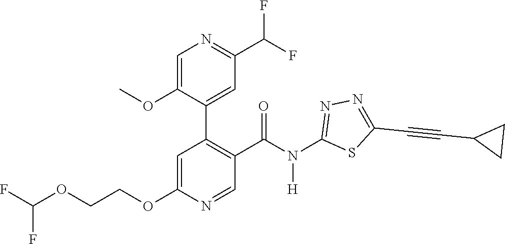 [SMILES: COc1cnc(C(F)F)cc1-c1cc(OCCOC(F)F)ncc1C(=O)Nc1nnc(C#CC2CC2)s1] | 181 |
| 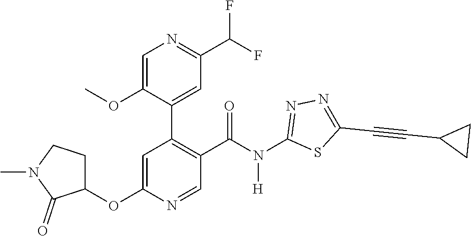 [SMILES: COc1cnc(C(F)F)cc1-c1cc(OC2CCN(C)C2=O)ncc1C(=O)Nc1nnc(C#CC2CC2)s1] | 182 |
| 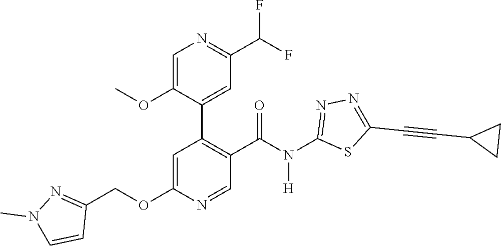 [SMILES: COc1cnc(C(F)F)cc1-c1cc(OCc2ccn(C)n2)ncc1C(=O)Nc1nnc(C#CC2CC2)s1] | 183 |
| 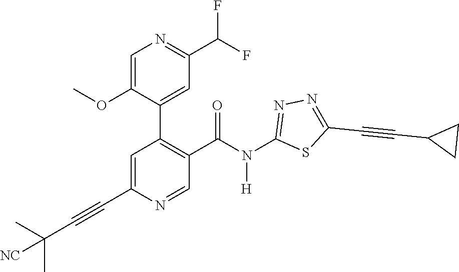 [SMILES: COc1cnc(C(F)F)cc1-c1cc(C#CC(C)(C)C#N)ncc1C(=O)Nc1nnc(C#CC2CC2)s1] | 184 |
| 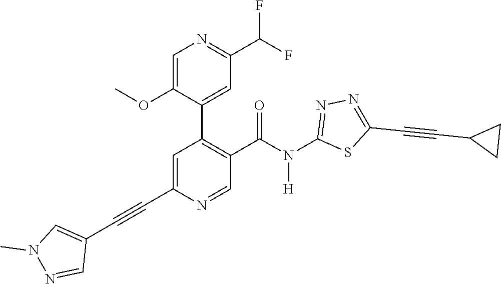 [SMILES: COc1cnc(C(F)F)cc1-c1cc(C#Cc2cnn(C)c2)ncc1C(=O)Nc1nnc(C#CC2CC2)s1] | 185 |
| 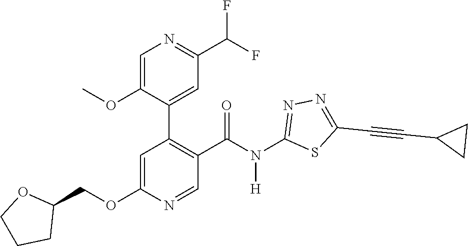 [SMILES: COc1cnc(C(F)F)cc1-c1cc(OC[C@H]2CCCO2)ncc1C(=O)Nc1nnc(C#CC2CC2)s1] | 186 |
| 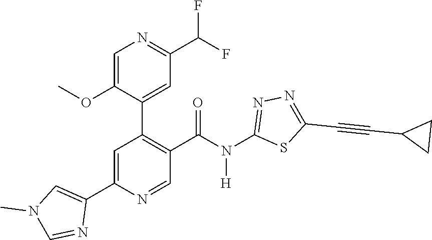 [SMILES: COc1cnc(C(F)F)cc1-c1cc(-c2cn(C)cn2)ncc1C(=O)Nc1nnc(C#CC2CC2)s1] | 187 |
| 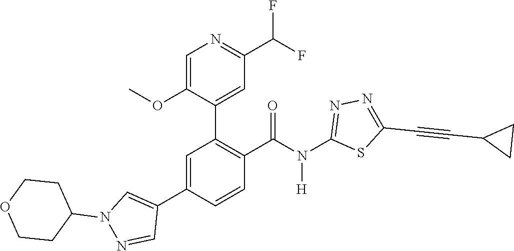 [SMILES: COc1cnc(C(F)F)cc1-c1cc(-c2cnn(C3CCOCC3)c2)ccc1C(=O)Nc1nnc(C#CC2CC2)s1] | 188 |
| 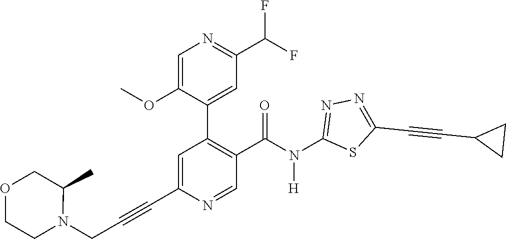 [SMILES: COc1cnc(C(F)F)cc1-c1cc(C#CCN2CCOC[C@H]2C)ncc1C(=O)Nc1nnc(C#CC2CC2)s1] | 189 |
| 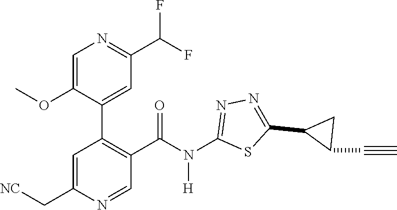 | 190 |
| 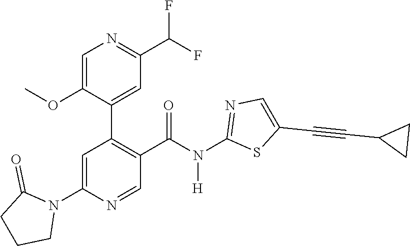 [SMILES: COc1cnc(C(F)F)cc1-c1cc(N2CCCC2=O)ncc1C(=O)Nc1ncc(C#CC2CC2)s1] | 191 |
| 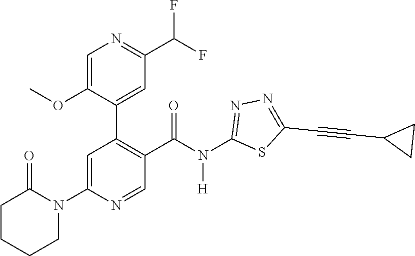 [SMILES: COc1cnc(C(F)F)cc1-c1cc(N2CCCCC2=O)ncc1C(=O)Nc1nnc(C#CC2CC2)s1] | 192 |
| 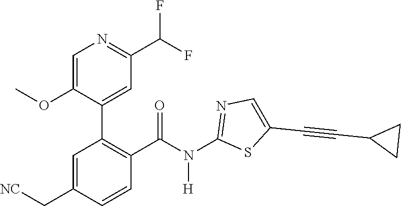 [SMILES: COc1cnc(C(F)F)cc1-c1cc(CC#N)ccc1C(=O)Nc1ncc(C#CC2CC2)s1] | 193 |
| 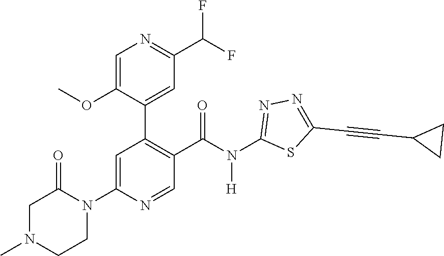 [SMILES: COc1cnc(C(F)F)cc1-c1cc(N2CCN(C)CC2=O)ncc1C(=O)Nc1nnc(C#CC2CC2)s1] | 194 |
| 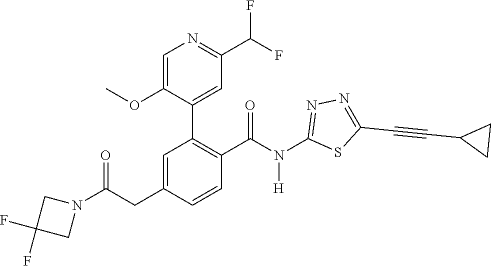 [SMILES: COc1cnc(C(F)F)cc1-c1cc(CC(=O)N2CC(F)(F)C2)ccc1C(=O)Nc1nnc(C#CC2CC2)s1] | 195 |
| 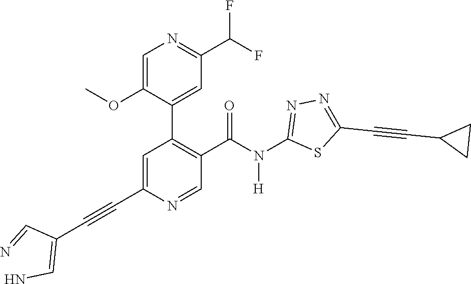 [SMILES: COc1cnc(C(F)F)cc1-c1cc(C#C/C(C=N)=C/N)ncc1C(=O)Nc1nnc(C#CC2CC2)s1] | 196 |
| 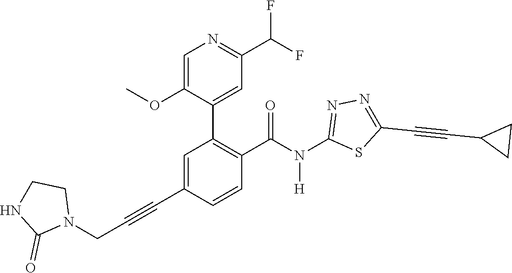 [SMILES: COc1cnc(C(F)F)cc1-c1cc(C#CCN2CCNC2=O)ccc1C(=O)Nc1nnc(C#CC2CC2)s1] | 197 |
| 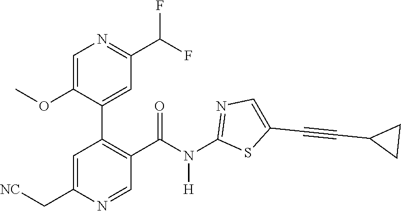 [SMILES: COc1cnc(C(F)F)cc1-c1cc(CC#N)ncc1C(=O)Nc1ncc(C#CC2CC2)s1] | 198 |
| 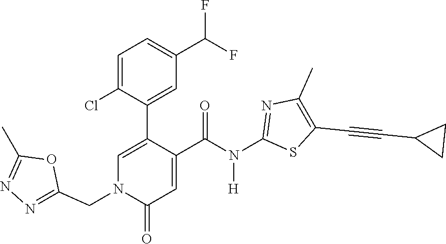 [SMILES: Cc1nnc(Cn2cc(-c3cc(C(F)F)ccc3Cl)c(C(=O)Nc3nc(C)c(C#CC4CC4)s3)cc2=O)o1] | 199 |
| 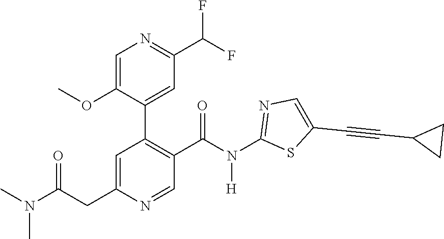 [SMILES: COc1cnc(C(F)F)cc1-c1cc(CC(=O)N(C)C)ncc1C(=O)Nc1ncc(C#CC2CC2)s1] | 200 |
| 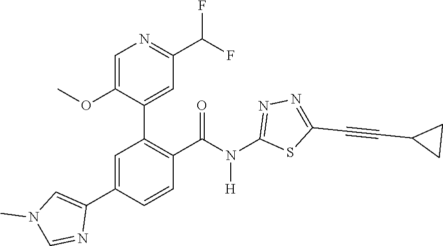 [SMILES: COc1cnc(C(F)F)cc1-c1cc(-c2cn(C)cn2)ccc1C(=O)Nc1nnc(C#CC2CC2)s1] | 201 |
| 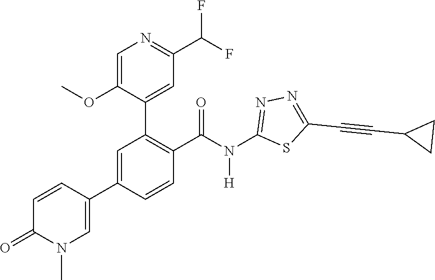 [SMILES: COc1cnc(C(F)F)cc1-c1cc(-c2ccc(=O)n(C)c2)ccc1C(=O)Nc1nnc(C#CC2CC2)s1] | 202 |
| 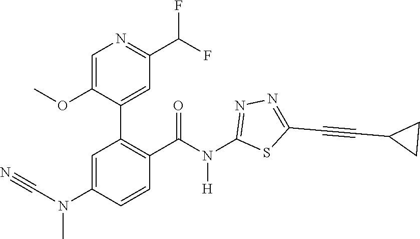 [SMILES: COc1cnc(C(F)F)cc1-c1cc(N(C)C#N)ccc1C(=O)Nc1nnc(C#CC2CC2)s1] | 203 |
| 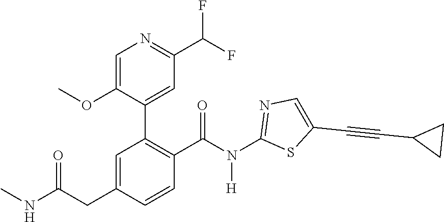 [SMILES: CNC(=O)Cc1ccc(C(=O)Nc2ncc(C#CC3CC3)s2)c(-c2cc(C(F)F)ncc2OC)c1] | 204 |
| 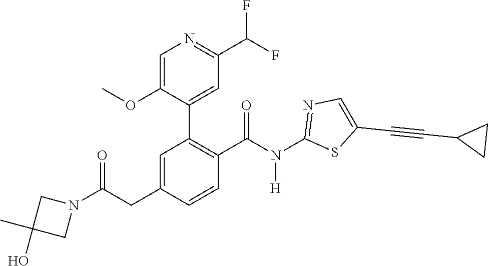 [SMILES: COc1cnc(C(F)F)cc1-c1cc(CC(=O)N2CC(C)(O)C2)ccc1C(=O)Nc1ncc(C#CC2CC2)s1] | 205 |
| 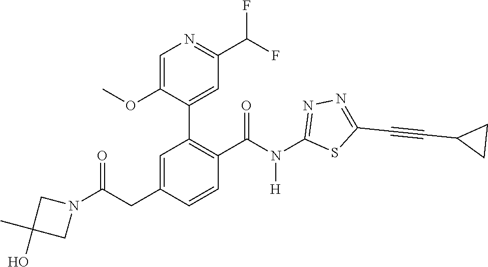 [SMILES: COc1cnc(C(F)F)cc1-c1cc(CC(=O)N2CC(C)(O)C2)ccc1C(=O)Nc1nnc(C#CC2CC2)s1] | 206 |
| 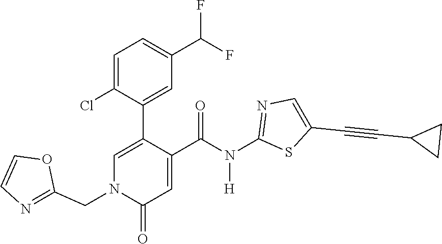 [SMILES: O=C(Nc1ncc(C#CC2CC2)s1)c1cc(=O)n(Cc2ncco2)cc1-c1cc(C(F)F)ccc1Cl] | 207 |
| 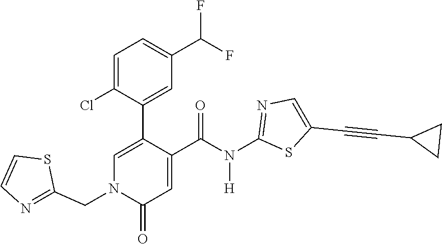 [SMILES: O=C(Nc1ncc(C#CC2CC2)s1)c1cc(=O)n(Cc2nccs2)cc1-c1cc(C(F)F)ccc1Cl] | 208 |
| 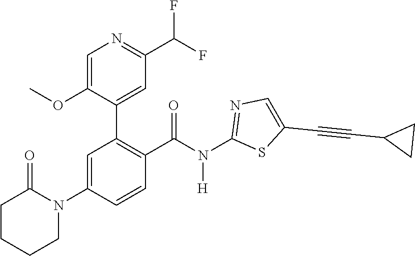 [SMILES: COc1cnc(C(F)F)cc1-c1cc(N2CCCCC2=O)ccc1C(=O)Nc1ncc(C#CC2CC2)s1] | 209 |
| 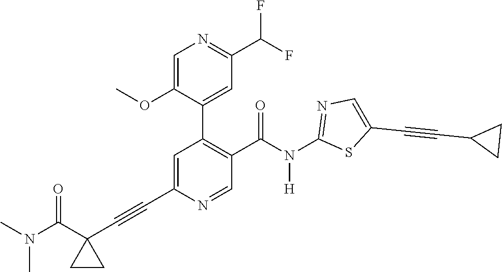 [SMILES: COc1cnc(C(F)F)cc1-c1cc(C#CC2(C(=O)N(C)C)CC2)ncc1C(=O)Nc1ncc(C#CC2CC2)s1] | 210 |
| 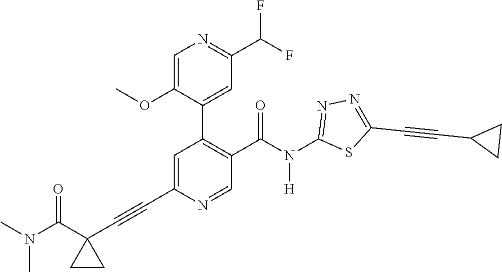 [SMILES: COc1cnc(C(F)F)cc1-c1cc(C#CC2(C(=O)N(C)C)CC2)ncc1C(=O)Nc1nnc(C#CC2CC2)s1] | 211 |
| 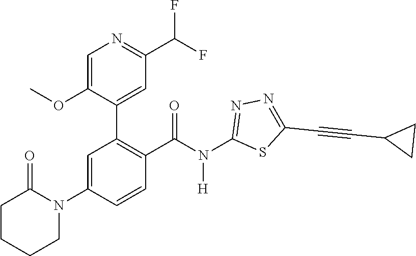 [SMILES: COc1cnc(C(F)F)cc1-c1cc(N2CCCCC2=O)ccc1C(=O)Nc1nnc(C#CC2CC2)s1] | 212 |
| 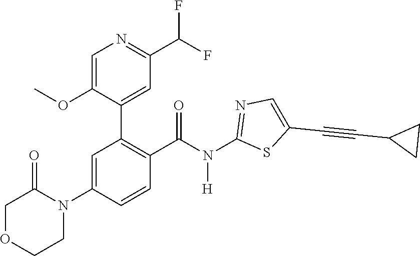 [SMILES: COc1cnc(C(F)F)cc1-c1cc(N2CCOCC2=O)ccc1C(=O)Nc1ncc(C#CC2CC2)s1] | 213 |
| 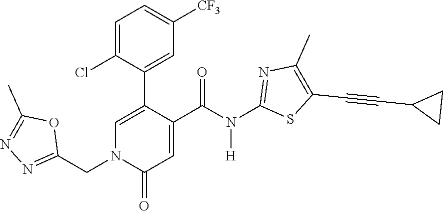 [SMILES: Cc1nnc(Cn2cc(-c3cc(C(F)(F)F)ccc3Cl)c(C(=O)Nc3nc(C)c(C#CC4CC4)s3)cc2=O)o1] | 214 |
| 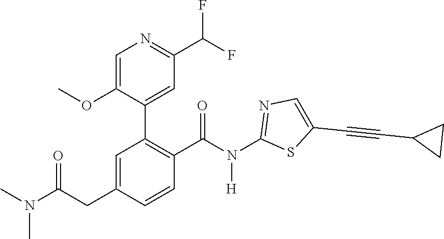 [SMILES: COc1cnc(C(F)F)cc1-c1cc(CC(=O)N(C)C)ccc1C(=O)Nc1ncc(C#CC2CC2)s1] | 215 |
| 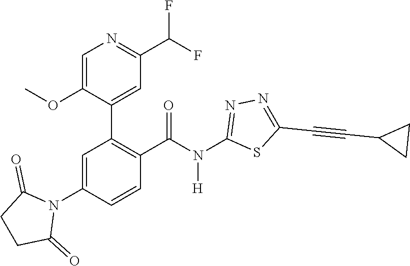 [SMILES: COc1cnc(C(F)F)cc1-c1cc(N2C(=O)CCC2=O)ccc1C(=O)Nc1nnc(C#CC2CC2)s1] | 216 |
| 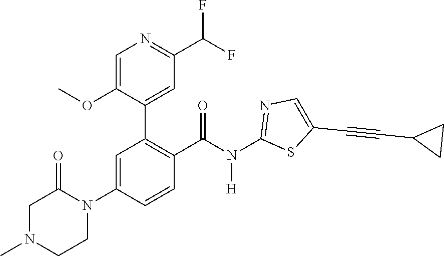 [SMILES: COc1cnc(C(F)F)cc1-c1cc(N2CCN(C)CC2=O)ccc1C(=O)Nc1ncc(C#CC2CC2)s1] | 217 |
| 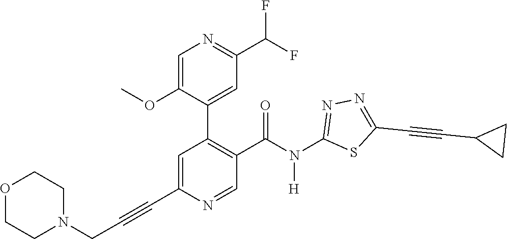 [SMILES: COc1cnc(C(F)F)cc1-c1cc(C#CCN2CCOCC2)ncc1C(=O)Nc1nnc(C#CC2CC2)s1] | 218 |
| 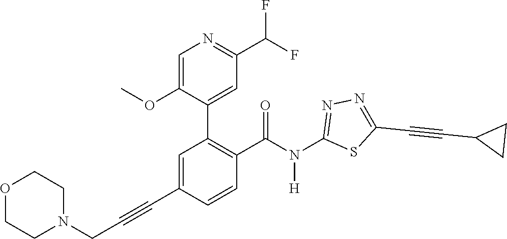 [SMILES: COc1cnc(C(F)F)cc1-c1cc(C#CCN2CCOCC2)ccc1C(=O)Nc1nnc(C#CC2CC2)s1] | 219 |
| 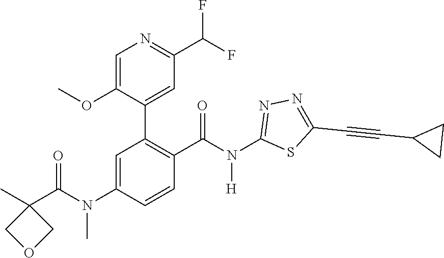 [SMILES: COc1cnc(C(F)F)cc1-c1cc(N(C)C(=O)C2(C)COC2)ccc1C(=O)Nc1nnc(C#CC2CC2)s1] | 220 |
| 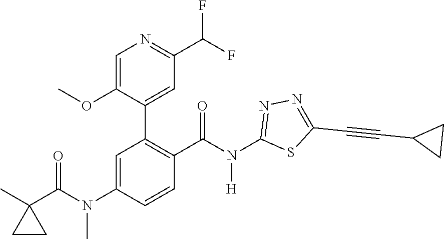 [SMILES: COc1cnc(C(F)F)cc1-c1cc(N(C)C(=O)C2(C)CC2)ccc1C(=O)Nc1nnc(C#CC2CC2)s1] | 221 |
| 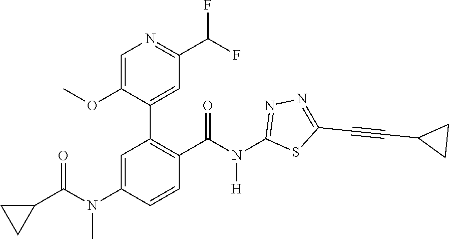 [SMILES: COc1cnc(C(F)F)cc1-c1cc(N(C)C(=O)C2CC2)ccc1C(=O)Nc1nnc(C#CC2CC2)s1] | 222 |
| 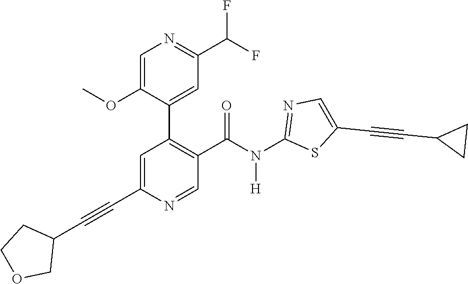 [SMILES: COc1cnc(C(F)F)cc1-c1cc(C#CC2CCOC2)ncc1C(=O)Nc1ncc(C#CC2CC2)s1] | 223 |
| 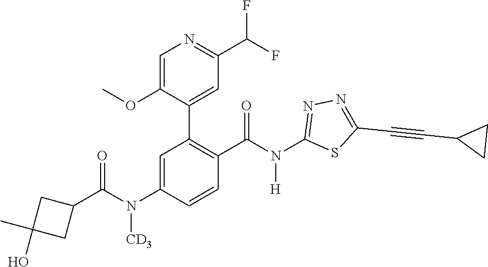 [SMILES: [2H]C([2H])([2H])N(C(=O)C1CC(C)(O)C1)c1ccc(C(=O)Nc2nnc(C#CC3CC3)s2)c(-c2cc(C(F)F)ncc2OC)c1] | 224 |
| 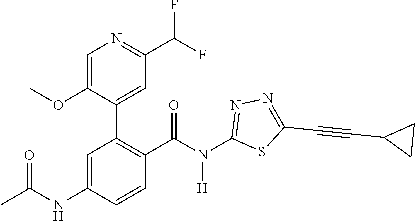 [SMILES: COc1cnc(C(F)F)cc1-c1cc(NC(C)=O)ccc1C(=O)Nc1nnc(C#CC2CC2)s1] | 225 |
| 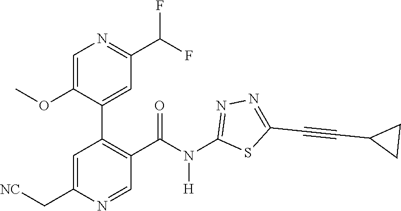 [SMILES: COc1cnc(C(F)F)cc1-c1cc(CC#N)ncc1C(=O)Nc1nnc(C#CC2CC2)s1] | 226 |
| 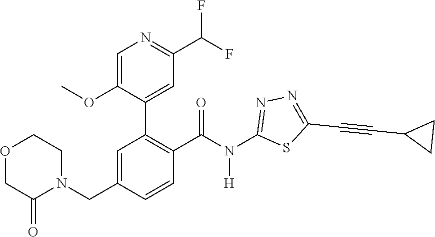 [SMILES: COc1cnc(C(F)F)cc1-c1cc(CN2CCOCC2=O)ccc1C(=O)Nc1nnc(C#CC2CC2)s1] | 227 |
| 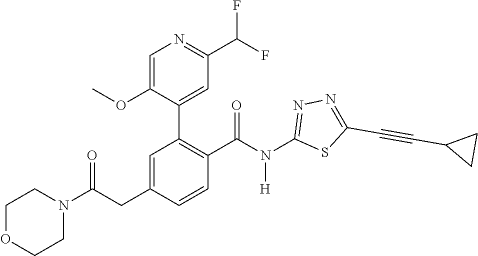 [SMILES: COc1cnc(C(F)F)cc1-c1cc(CC(=O)N2CCOCC2)ccc1C(=O)Nc1nnc(C#CC2CC2)s1] | 228 |
| 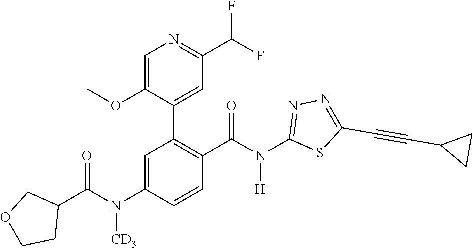 [SMILES: [2H]C([2H])([2H])N(C(=O)C1CCOC1)c1ccc(C(=O)Nc2nnc(C#CC3CC3)s2)c(-c2cc(C(F)F)ncc2OC)c1] | 229 |
| 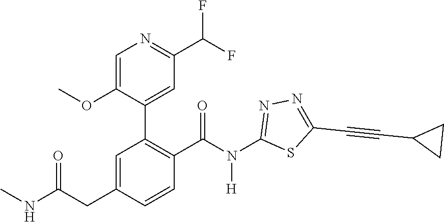 [SMILES: CNC(=O)Cc1ccc(C(=O)Nc2nnc(C#CC3CC3)s2)c(-c2cc(C(F)F)ncc2OC)c1] | 230 |
| 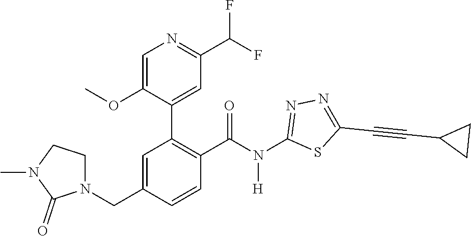 [SMILES: COc1cnc(C(F)F)cc1-c1cc(CN2CCN(C)C2=O)ccc1C(=O)Nc1nnc(C#CC2CC2)s1] | 231 |
| 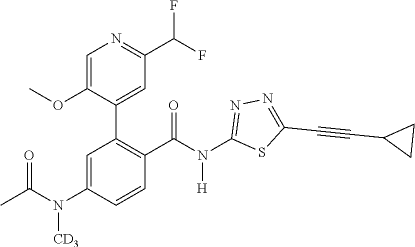 [SMILES: [2H]C([2H])([2H])N(C(C)=O)c1ccc(C(=O)Nc2nnc(C#CC3CC3)s2)c(-c2cc(C(F)F)ncc2OC)c1] | 232 |
| 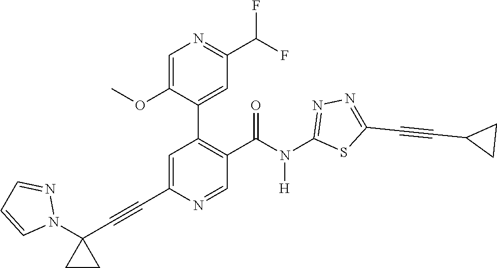 [SMILES: COc1cnc(C(F)F)cc1-c1cc(C#CC2(n3cccn3)CC2)ncc1C(=O)Nc1nnc(C#CC2CC2)s1] | 233 |
| 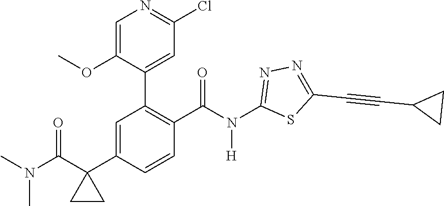 [SMILES: COc1cnc(Cl)cc1-c1cc(C2(C(=O)N(C)C)CC2)ccc1C(=O)Nc1nnc(C#CC2CC2)s1] | 234 |
| 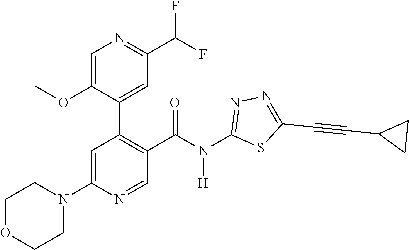 [SMILES: COc1cnc(C(F)F)cc1-c1cc(N2CCOCC2)ncc1C(=O)Nc1nnc(C#CC2CC2)s1] | 235 |
| 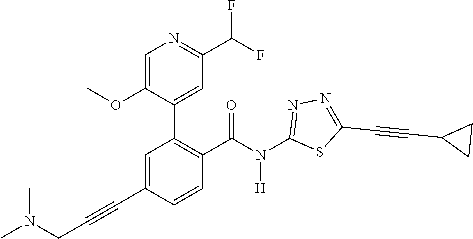 [SMILES: COc1cnc(C(F)F)cc1-c1cc(C#CCN(C)C)ccc1C(=O)Nc1nnc(C#CC2CC2)s1] | 236 |
| 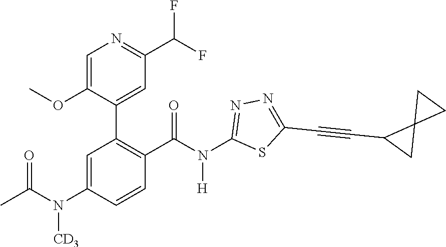 [SMILES: [2H]C([2H])([2H])N(C(C)=O)c1ccc(C(=O)Nc2nnc(C#CC3CC34CC4)s2)c(-c2cc(C(F)F)ncc2OC)c1] | 237 |
| 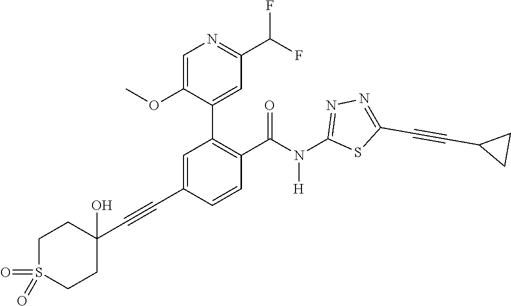 [SMILES: COc1cnc(C(F)F)cc1-c1cc(C#CC2(O)CCS(=O)(=O)CC2)ccc1C(=O)Nc1nnc(C#CC2CC2)s1] | 238 |
| 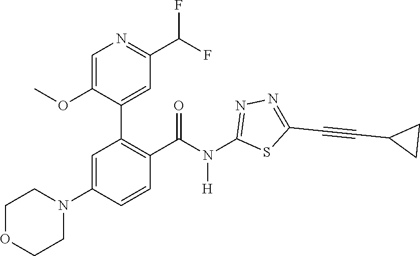 [SMILES: COc1cnc(C(F)F)cc1-c1cc(N2CCOCC2)ccc1C(=O)Nc1nnc(C#CC2CC2)s1] | 239 |
| 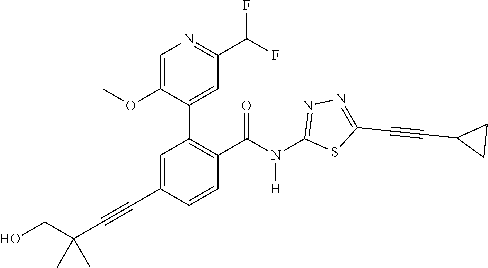 [SMILES: COc1cnc(C(F)F)cc1-c1cc(C#CC(C)(C)CO)ccc1C(=O)Nc1nnc(C#CC2CC2)s1] | 240 |
| 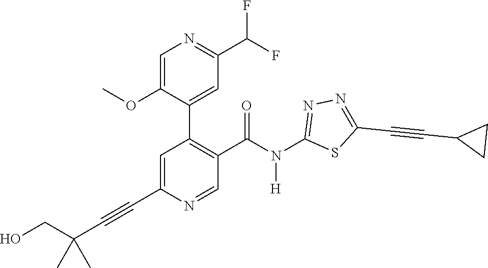 [SMILES: COc1cnc(C(F)F)cc1-c1cc(C#CC(C)(C)CO)ncc1C(=O)Nc1nnc(C#CC2CC2)s1] | 241 |
| 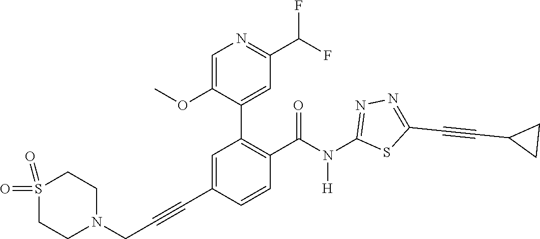 [SMILES: COc1cnc(C(F)F)cc1-c1cc(C#CCN2CCS(=O)(=O)CC2)ccc1C(=O)Nc1nnc(C#CC2CC2)s1] | 242 |
| 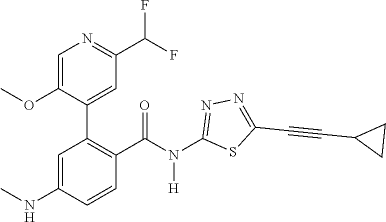 [SMILES: CNc1ccc(C(=O)Nc2nnc(C#CC3CC3)s2)c(-c2cc(C(F)F)ncc2OC)c1] | 243 |
| 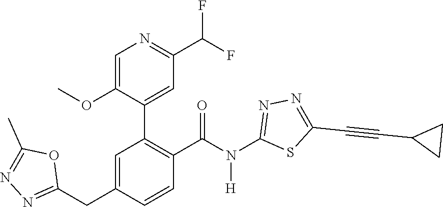 [SMILES: COc1cnc(C(F)F)cc1-c1cc(Cc2nnc(C)o2)ccc1C(=O)Nc1nnc(C#CC2CC2)s1] | 244 |
| 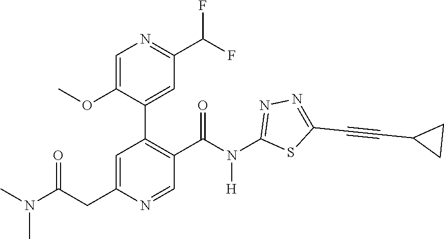 [SMILES: COc1cnc(C(F)F)cc1-c1cc(CC(=O)N(C)C)ncc1C(=O)Nc1nnc(C#CC2CC2)s1] | 245 |
| 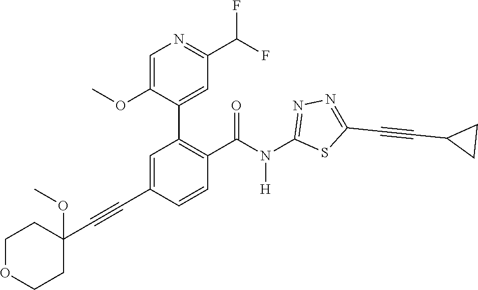 [SMILES: COc1cnc(C(F)F)cc1-c1cc(C#CC2(OC)CCOCC2)ccc1C(=O)Nc1nnc(C#CC2CC2)s1] | 246 |
| 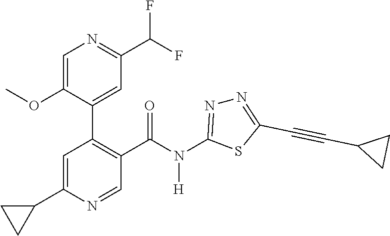 [SMILES: COc1cnc(C(F)F)cc1-c1cc(C2CC2)ncc1C(=O)Nc1nnc(C#CC2CC2)s1] | 247 |
| 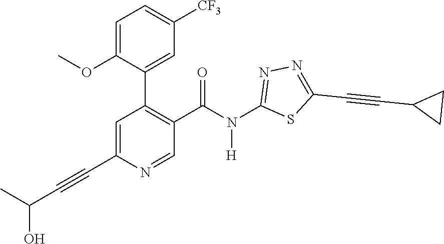 [SMILES: COc1ccc(C(F)(F)F)cc1-c1cc(C#CC(C)O)ncc1C(=O)Nc1nnc(C#CC2CC2)s1] | 248 |
| 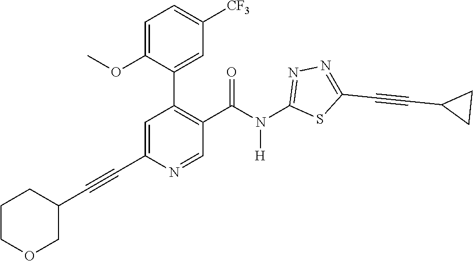 [SMILES: COc1ccc(C(F)(F)F)cc1-c1cc(C#CC2CCCOC2)ncc1C(=O)Nc1nnc(C#CC2CC2)s1] | 249 |
| 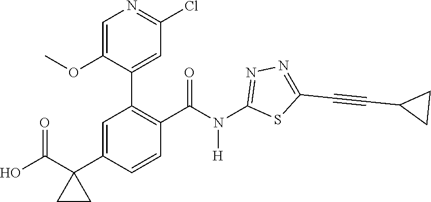 [SMILES: COc1cnc(Cl)cc1-c1cc(C2(C(=O)O)CC2)ccc1C(=O)Nc1nnc(C#CC2CC2)s1] | 250 |
| 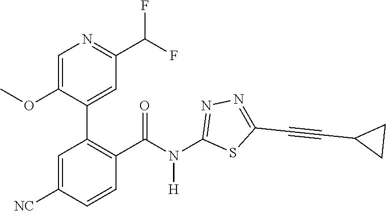 [SMILES: COc1cnc(C(F)F)cc1-c1cc(C#N)ccc1C(=O)Nc1nnc(C#CC2CC2)s1] | 251 |
| 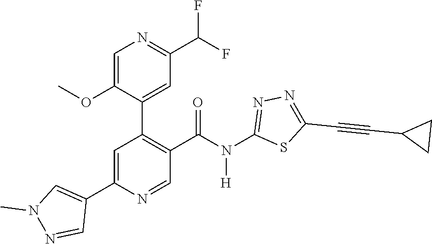 [SMILES: COc1cnc(C(F)F)cc1-c1cc(-c2cnn(C)c2)ncc1C(=O)Nc1nnc(C#CC2CC2)s1] | 252 |
| 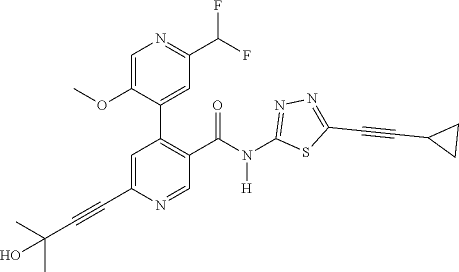 [SMILES: COc1cnc(C(F)F)cc1-c1cc(C#CC(C)(C)O)ncc1C(=O)Nc1nnc(C#CC2CC2)s1] | 253 |
| 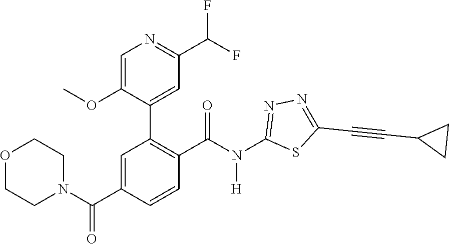 [SMILES: COc1cnc(C(F)F)cc1-c1cc(C(=O)N2CCOCC2)ccc1C(=O)Nc1nnc(C#CC2CC2)s1] | 254 |
| 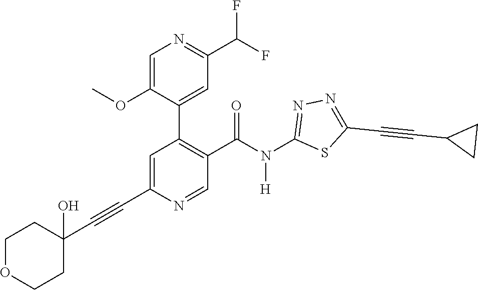 [SMILES: COc1cnc(C(F)F)cc1-c1cc(C#CC2(O)CCOCC2)ncc1C(=O)Nc1nnc(C#CC2CC2)s1] | 255 |
| 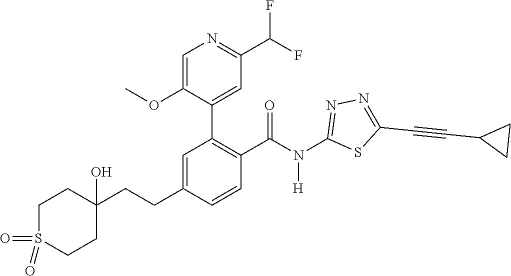 [SMILES: COc1cnc(C(F)F)cc1-c1cc(CCC2(O)CCS(=O)(=O)CC2)ccc1C(=O)Nc1nnc(C#CC2CC2)s1] | 256 |
| 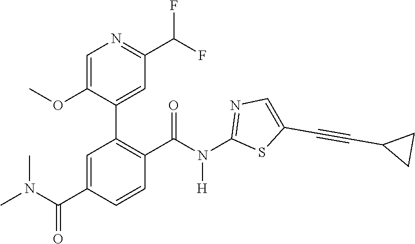 [SMILES: COc1cnc(C(F)F)cc1-c1cc(C(=O)N(C)C)ccc1C(=O)Nc1ncc(C#CC2CC2)s1] | 257 |
| 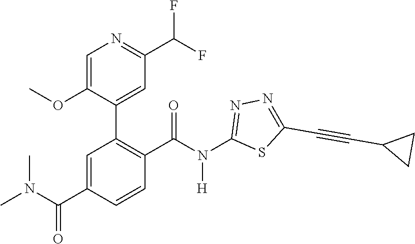 [SMILES: COc1cnc(C(F)F)cc1-c1cc(C(=O)N(C)C)ccc1C(=O)Nc1nnc(C#CC2CC2)s1] | 258 |
| 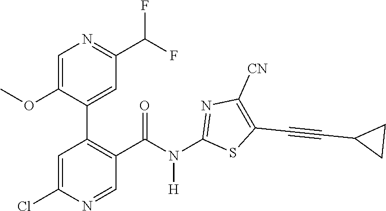 [SMILES: COc1cnc(C(F)F)cc1-c1cc(Cl)ncc1C(=O)Nc1nc(C#N)c(C#CC2CC2)s1] | 259 |
| 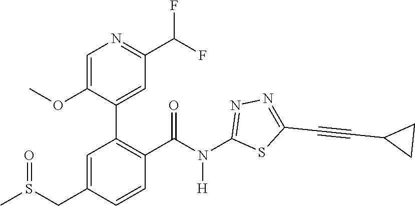 [SMILES: COc1cnc(C(F)F)cc1-c1cc(CS(C)=O)ccc1C(=O)Nc1nnc(C#CC2CC2)s1] | 260 |
| 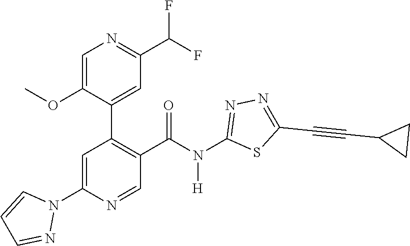 [SMILES: COc1cnc(C(F)F)cc1-c1cc(-n2cccn2)ncc1C(=O)Nc1nnc(C#CC2CC2)s1] | 261 |
| 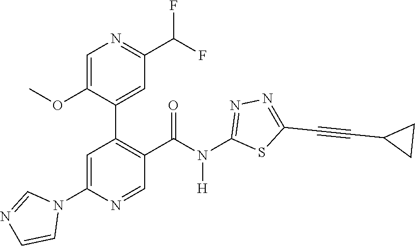 [SMILES: COc1cnc(C(F)F)cc1-c1cc(-n2ccnc2)ncc1C(=O)Nc1nnc(C#CC2CC2)s1] | 262 |
| 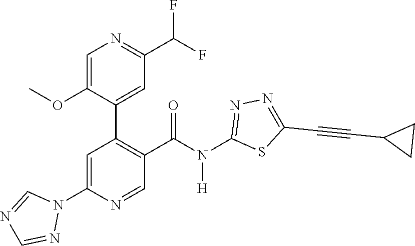 [SMILES: COc1cnc(C(F)F)cc1-c1cc(-n2cncn2)ncc1C(=O)Nc1nnc(C#CC2CC2)s1] | 263 |
| 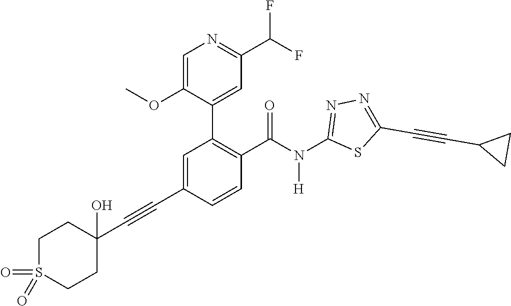 [SMILES: COc1cnc(C(F)F)cc1-c1cc(C#CC2(O)CCS(=O)(=O)CC2)ccc1C(=O)Nc1nnc(C#CC2CC2)s1] | 264 |
| 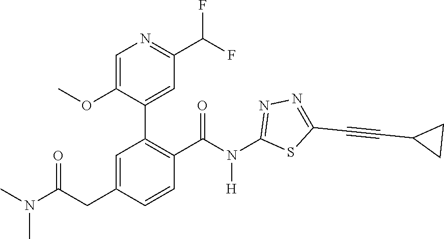 [SMILES: COc1cnc(C(F)F)cc1-c1cc(CC(=O)N(C)C)ccc1C(=O)Nc1nnc(C#CC2CC2)s1] | 265 |
| 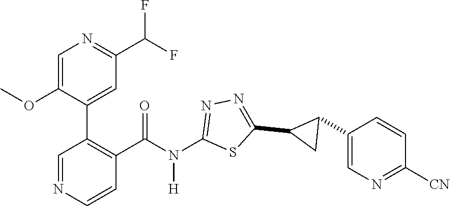 | 266 |
| 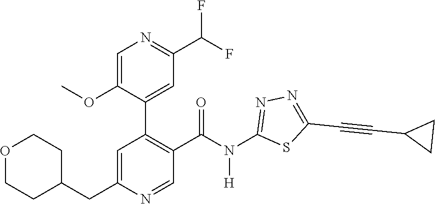 [SMILES: COc1cnc(C(F)F)cc1-c1cc(CC2CCOCC2)ncc1C(=O)Nc1nnc(C#CC2CC2)s1] | 267 |
| 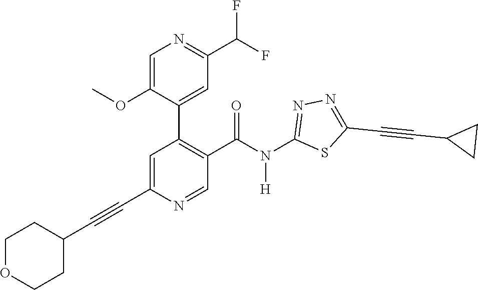 [SMILES: COc1cnc(C(F)F)cc1-c1cc(C#CC2CCOCC2)ncc1C(=O)Nc1nnc(C#CC2CC2)s1] | 268 |
| 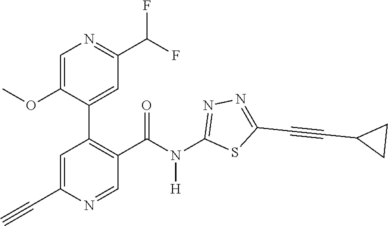 [SMILES: C#Cc1cc(-c2cc(C(F)F)ncc2OC)c(C(=O)Nc2nnc(C#CC3CC3)s2)cn1] | 269 |
| 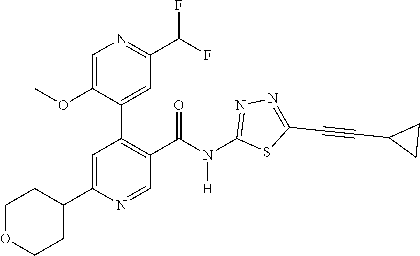 [SMILES: COc1cnc(C(F)F)cc1-c1cc(C2CCOCC2)ncc1C(=O)Nc1nnc(C#CC2CC2)s1] | 270 |
| 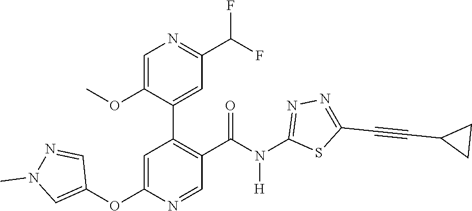 [SMILES: COc1cnc(C(F)F)cc1-c1cc(Oc2cnn(C)c2)ncc1C(=O)Nc1nnc(C#CC2CC2)s1] | 271 |
| 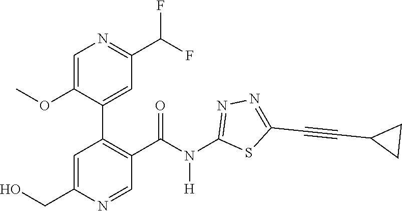 [SMILES: COc1cnc(C(F)F)cc1-c1cc(CO)ncc1C(=O)Nc1nnc(C#CC2CC2)s1] | 272 |
| 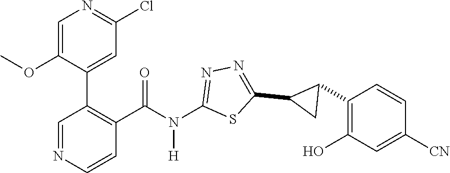 | 273 |
| 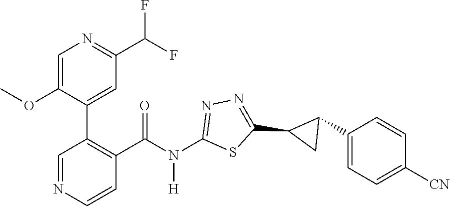 [SMILES: COc1cnc(C(F)F)cc1-c1cnccc1C(=O)Nc1nnc([C@@H]2C[C@H]2c2ccc(C#N)cc2)s1] | 274 |
| 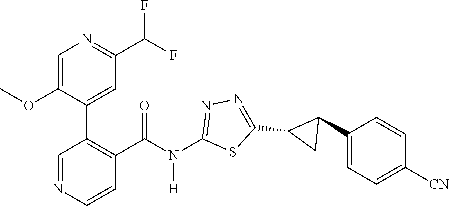 [SMILES: COc1cnc(C(F)F)cc1-c1cnccc1C(=O)Nc1nnc([C@H]2C[C@@H]2c2ccc(C#N)cc2)s1] | 275 |
| 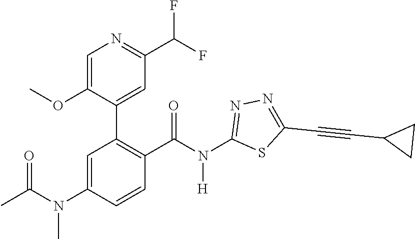 [SMILES: COc1cnc(C(F)F)cc1-c1cc(N(C)C(C)=O)ccc1C(=O)Nc1nnc(C#CC2CC2)s1] | 276 |
| 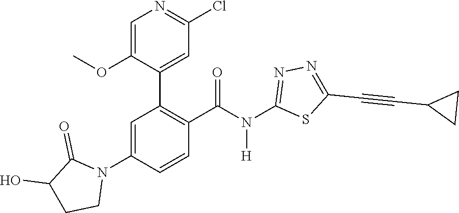 [SMILES: COc1cnc(Cl)cc1-c1cc(N2CCC(O)C2=O)ccc1C(=O)Nc1nnc(C#CC2CC2)s1] | 277 |
| 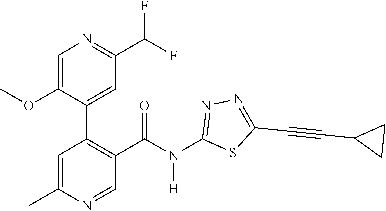 [SMILES: COc1cnc(C(F)F)cc1-c1cc(C)ncc1C(=O)Nc1nnc(C#CC2CC2)s1] | 278 |
| 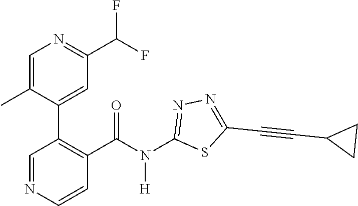 [SMILES: Cc1cnc(C(F)F)cc1-c1cnccc1C(=O)Nc1nnc(C#CC2CC2)s1] | 279 |
| 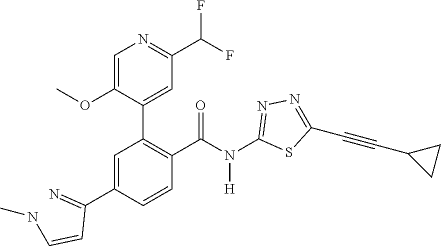 [SMILES: CN/C=C\C(=N)c1ccc(C(=O)Nc2nnc(C#CC3CC3)s2)c(-c2cc(C(F)F)ncc2OC)c1] | 280 |
| 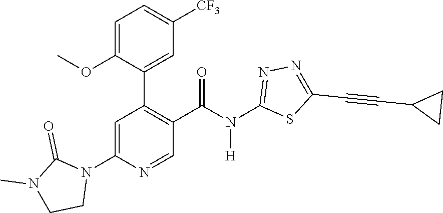 [SMILES: COc1ccc(C(F)(F)F)cc1-c1cc(N2CCN(C)C2=O)ncc1C(=O)Nc1nnc(C#CC2CC2)s1] | 281 |
| 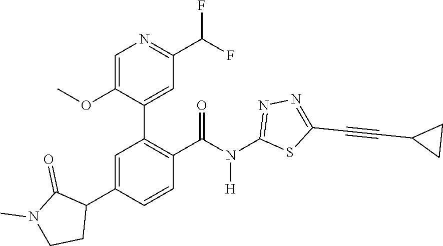 [SMILES: COc1cnc(C(F)F)cc1-c1cc(C2CCN(C)C2=O)ccc1C(=O)Nc1nnc(C#CC2CC2)s1] | 282 |
| 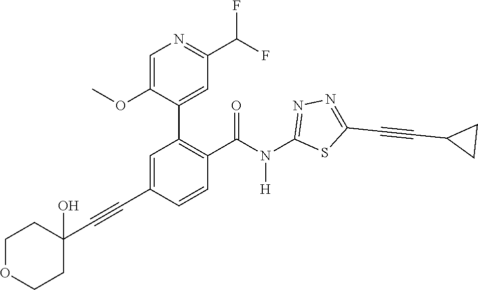 [SMILES: COc1cnc(C(F)F)cc1-c1cc(C#CC2(O)CCOCC2)ccc1C(=O)Nc1nnc(C#CC2CC2)s1] | 283 |
| 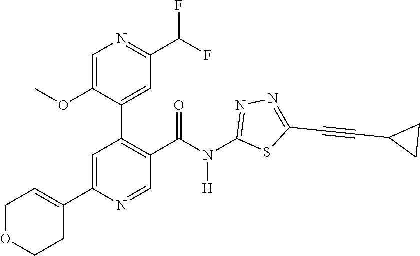 [SMILES: COc1cnc(C(F)F)cc1-c1cc(C2=CCOCC2)ncc1C(=O)Nc1nnc(C#CC2CC2)s1] | 284 |
| 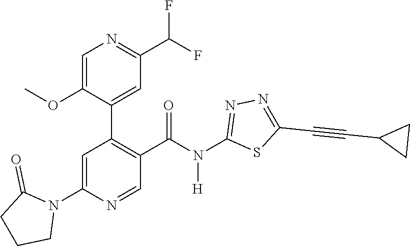 [SMILES: COc1cnc(C(F)F)cc1-c1cc(N2CCCC2=O)ncc1C(=O)Nc1nnc(C#CC2CC2)s1] | 285 |
| 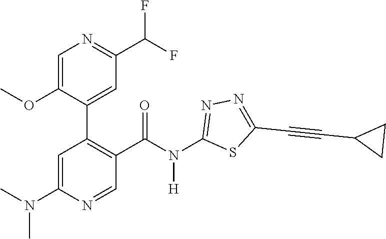 [SMILES: COc1cnc(C(F)F)cc1-c1cc(N(C)C)ncc1C(=O)Nc1nnc(C#CC2CC2)s1] | 286 |
| 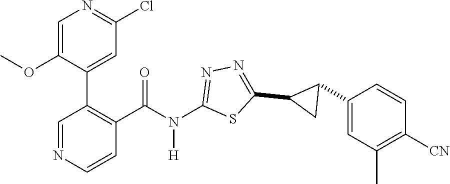 | 287 |
| 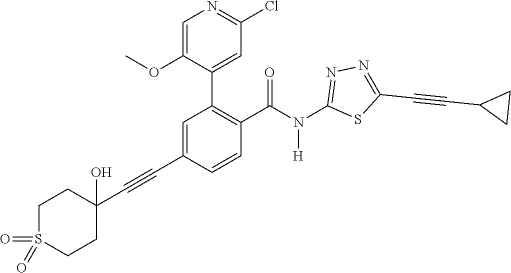 [SMILES: COc1cnc(Cl)cc1-c1cc(C#CC2(O)CCS(=O)(=O)CC2)ccc1C(=O)Nc1nnc(C#CC2CC2)s1] | 288 |
| 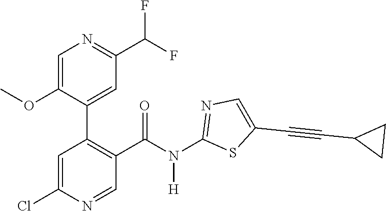 [SMILES: COc1cnc(C(F)F)cc1-c1cc(Cl)ncc1C(=O)Nc1ncc(C#CC2CC2)s1] | 289 |
| 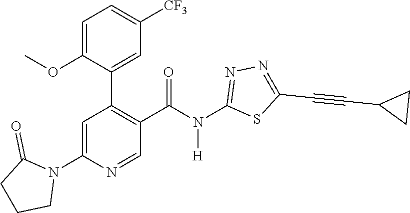 [SMILES: COc1ccc(C(F)(F)F)cc1-c1cc(N2CCCC2=O)ncc1C(=O)Nc1nnc(C#CC2CC2)s1] | 290 |
| 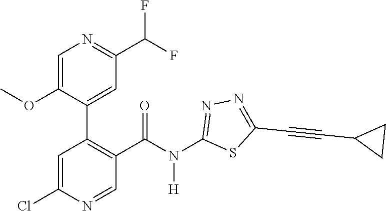 [SMILES: COc1cnc(C(F)F)cc1-c1cc(Cl)ncc1C(=O)Nc1nnc(C#CC2CC2)s1] | 291 |
| 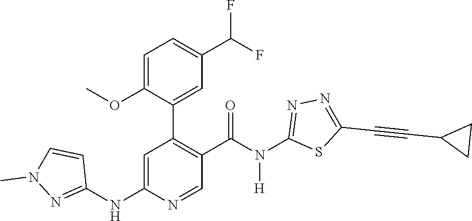 [SMILES: COc1ccc(C(F)F)cc1-c1cc(Nc2ccn(C)n2)ncc1C(=O)Nc1nnc(C#CC2CC2)s1] | 292 |
| 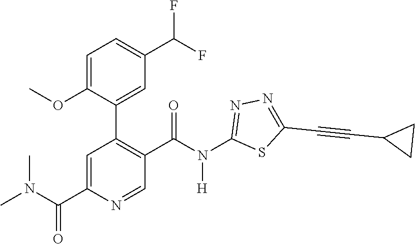 [SMILES: COc1ccc(C(F)F)cc1-c1cc(C(=O)N(C)C)ncc1C(=O)Nc1nnc(C#CC2CC2)s1] | 293 |
| 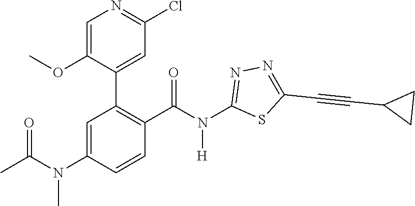 [SMILES: COc1cnc(Cl)cc1-c1cc(N(C)C(C)=O)ccc1C(=O)Nc1nnc(C#CC2CC2)s1] | 294 |
| 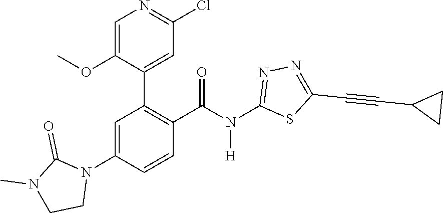 [SMILES: COc1cnc(Cl)cc1-c1cc(N2CCN(C)C2=O)ccc1C(=O)Nc1nnc(C#CC2CC2)s1] | 295 |
| 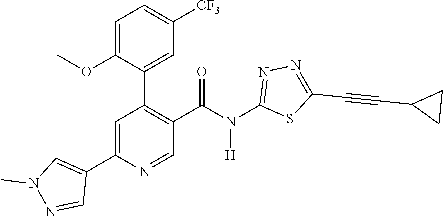 [SMILES: COc1ccc(C(F)(F)F)cc1-c1cc(-c2cnn(C)c2)ncc1C(=O)Nc1nnc(C#CC2CC2)s1] | 296 |
| 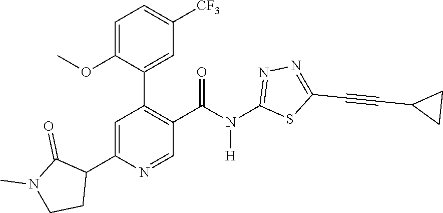 [SMILES: COc1ccc(C(F)(F)F)cc1-c1cc(C2CCN(C)C2=O)ncc1C(=O)Nc1nnc(C#CC2CC2)s1] | 297 |
| 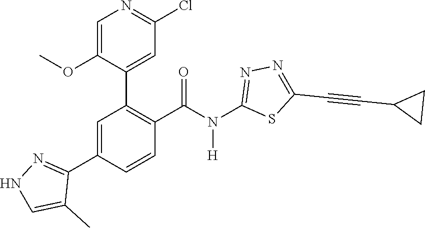 [SMILES: COc1cnc(Cl)cc1-c1cc(-c2n[nH]cc2C)ccc1C(=O)Nc1nnc(C#CC2CC2)s1] | 298 |
| 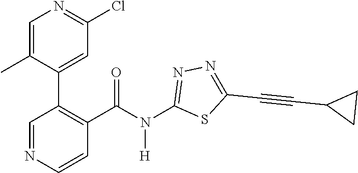 [SMILES: Cc1cnc(Cl)cc1-c1cnccc1C(=O)Nc1nnc(C#CC2CC2)s1] | 299 |
| 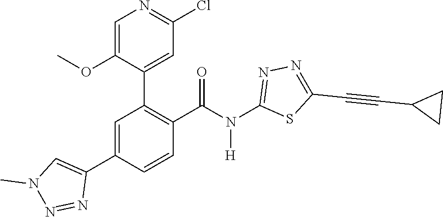 [SMILES: COc1cnc(Cl)cc1-c1cc(-c2cn(C)nn2)ccc1C(=O)Nc1nnc(C#CC2CC2)s1] | 300 |
| 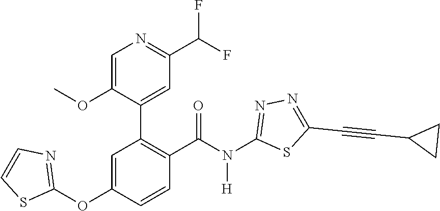 [SMILES: COc1cnc(C(F)F)cc1-c1cc(Oc2nccs2)ccc1C(=O)Nc1nnc(C#CC2CC2)s1] | 301 |
| 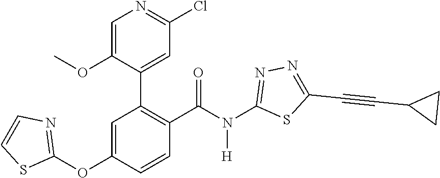 [SMILES: COc1cnc(Cl)cc1-c1cc(Oc2nccs2)ccc1C(=O)Nc1nnc(C#CC2CC2)s1] | 302 |
| 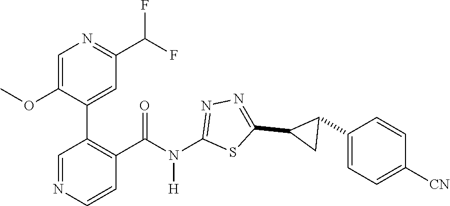 | 303 |
| 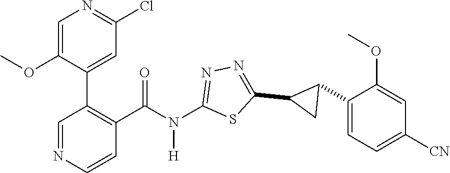 | 304 |
| 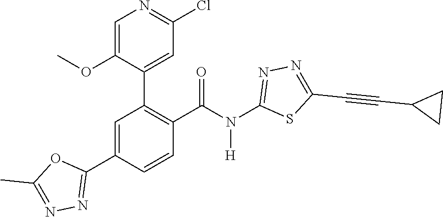 [SMILES: COc1cnc(Cl)cc1-c1cc(-c2nnc(C)o2)ccc1C(=O)Nc1nnc(C#CC2CC2)s1] | 305 |
| 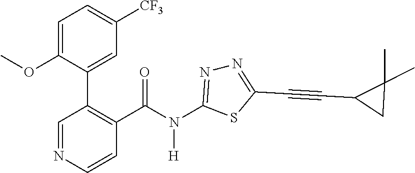 | 306 |
| 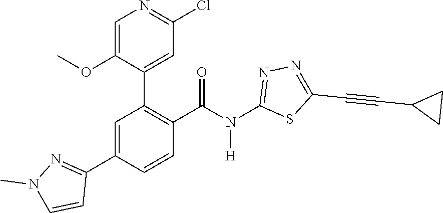 [SMILES: COc1cnc(Cl)cc1-c1cc(-c2ccn(C)n2)ccc1C(=O)Nc1nnc(C#CC2CC2)s1] | 307 |
| 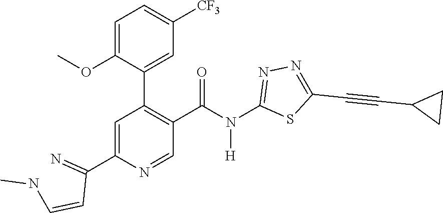 [SMILES: CN/C=C\C(=N)c1cc(-c2cc(C(F)(F)F)ccc2OC)c(C(=O)Nc2nnc(C#CC3CC3)s2)cn1] | 308 |
| 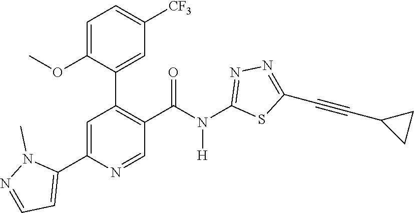 [SMILES: COc1ccc(C(F)(F)F)cc1-c1cc(-c2ccnn2C)ncc1C(=O)Nc1nnc(C#CC2CC2)s1] | 309 |
| 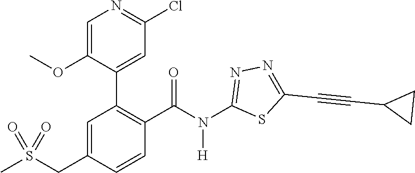 [SMILES: COc1cnc(Cl)cc1-c1cc(CS(C)(=O)=O)ccc1C(=O)Nc1nnc(C#CC2CC2)s1] | 310 |
| 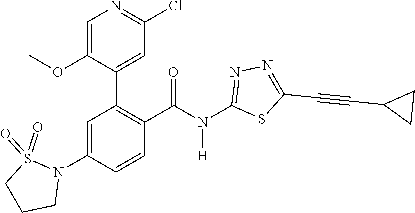 [SMILES: COc1cnc(Cl)cc1-c1cc(N2CCCS2(=O)=O)ccc1C(=O)Nc1nnc(C#CC2CC2)s1] | 311 |
| 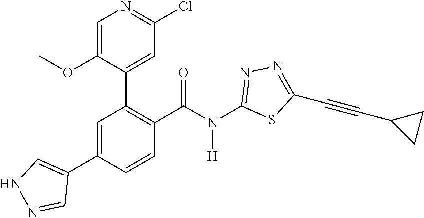 [SMILES: COc1cnc(Cl)cc1-c1cc(-c2cn[nH]c2)ccc1C(=O)Nc1nnc(C#CC2CC2)s1] | 312 |
| 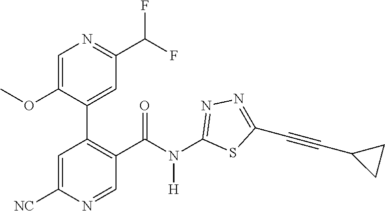 [SMILES: COc1cnc(C(F)F)cc1-c1cc(C#N)ncc1C(=O)Nc1nnc(C#CC2CC2)s1] | 313 |
| 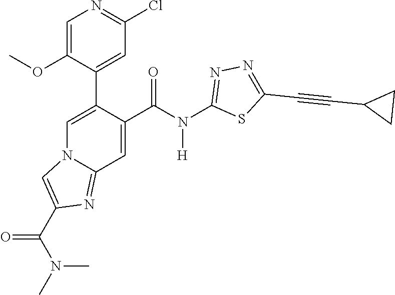 [SMILES: COc1cnc(Cl)cc1-c1cn2cc(C(=O)N(C)C)nc2cc1C(=O)Nc1nnc(C#CC2CC2)s1] | 314 |
| 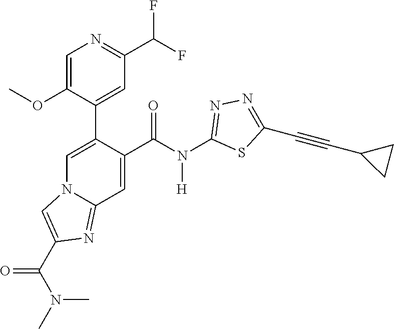 [SMILES: COc1cnc(C(F)F)cc1-c1cn2cc(C(=O)N(C)C)nc2cc1C(=O)Nc1nnc(C#CC2CC2)s1] | 315 |
| 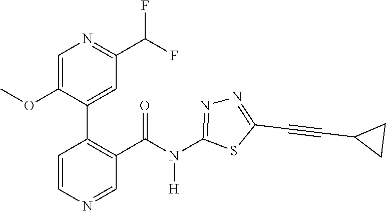 [SMILES: COc1cnc(C(F)F)cc1-c1ccncc1C(=O)Nc1nnc(C#CC2CC2)s1] | 316 |
| 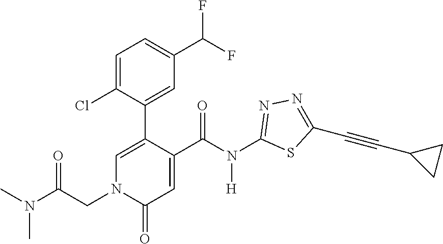 [SMILES: CN(C)C(=O)Cn1cc(-c2cc(C(F)F)ccc2Cl)c(C(=O)Nc2nnc(C#CC3CC3)s2)cc1=O] | 317 |
| 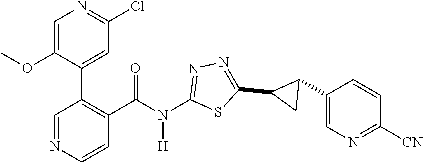 | 318 |
| 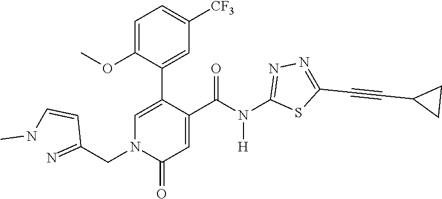 [SMILES: CN/C=C\C(=N)Cn1cc(-c2cc(C(F)(F)F)ccc2OC)c(C(=O)Nc2nnc(C#CC3CC3)s2)cc1=O] | 319 |
| 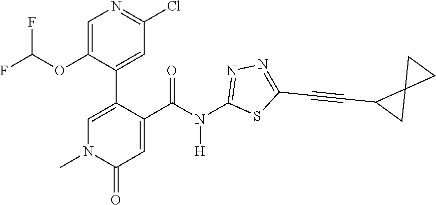 [SMILES: Cn1cc(-c2cc(Cl)ncc2OC(F)F)c(C(=O)Nc2nnc(C#CC3CC34CC4)s2)cc1=O] | 320 |
| 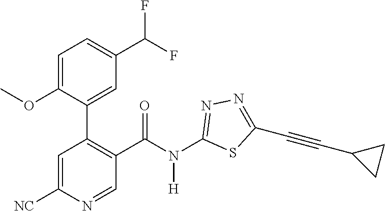 [SMILES: COc1ccc(C(F)F)cc1-c1cc(C#N)ncc1C(=O)Nc1nnc(C#CC2CC2)s1] | 321 |
| 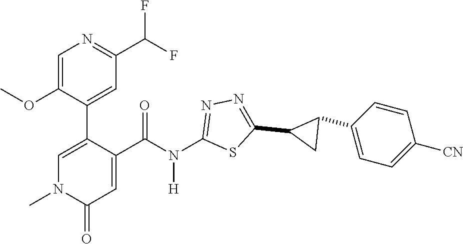 | 322 |
| 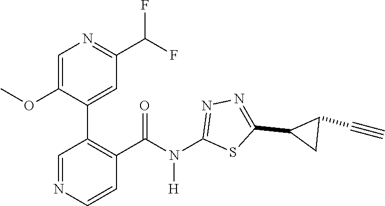 | 323 |
| 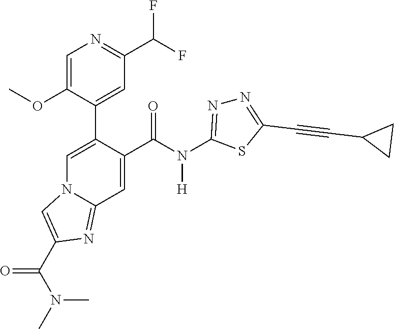 [SMILES: COc1cnc(C(F)F)cc1-c1cn2cc(C(=O)N(C)C)nc2cc1C(=O)Nc1nnc(C#CC2CC2)s1] | 324 |
| 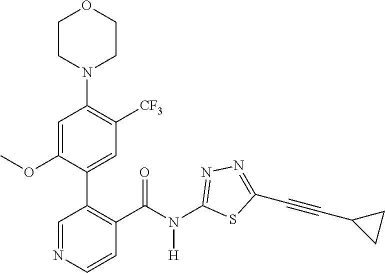 [SMILES: COc1cc(N2CCOCC2)c(C(F)(F)F)cc1-c1cnccc1C(=O)Nc1nnc(C#CC2CC2)s1] | 325 |
| 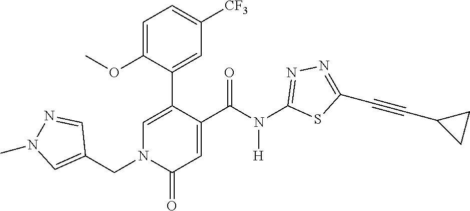 [SMILES: COc1ccc(C(F)(F)F)cc1-c1cn(Cc2cnn(C)c2)c(=O)cc1C(=O)Nc1nnc(C#CC2CC2)s1] | 326 |
| 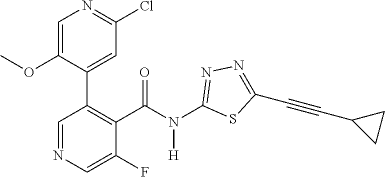 [SMILES: COc1cnc(Cl)cc1-c1cncc(F)c1C(=O)Nc1nnc(C#CC2CC2)s1] | 327 |
| 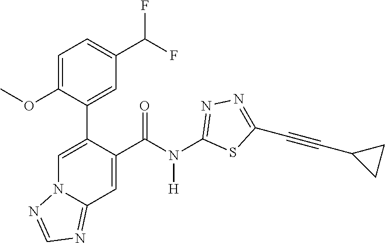 [SMILES: COc1ccc(C(F)F)cc1-c1cn2ncnc2cc1C(=O)Nc1nnc(C#CC2CC2)s1] | 328 |
| 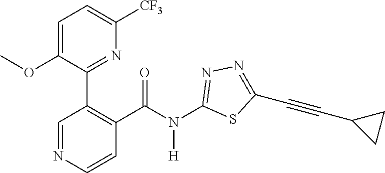 [SMILES: COc1ccc(C(F)(F)F)nc1-c1cnccc1C(=O)Nc1nnc(C#CC2CC2)s1] | 329 |
| 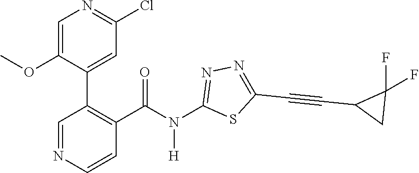 [SMILES: COc1cnc(Cl)cc1-c1cnccc1C(=O)Nc1nnc(C#CC2CC2(F)F)s1] | 330 |
| 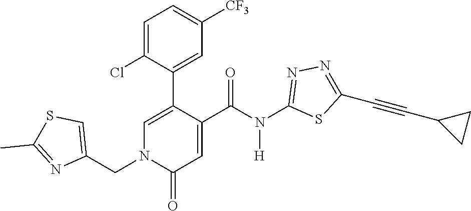 [SMILES: Cc1nc(Cn2cc(-c3cc(C(F)(F)F)ccc3Cl)c(C(=O)Nc3nnc(C#CC4CC4)s3)cc2=O)cs1] | 331 |
| 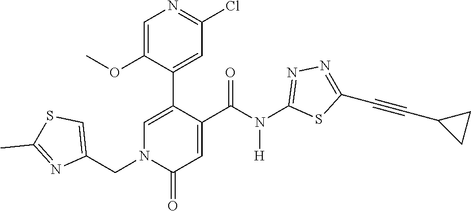 [SMILES: COc1cnc(Cl)cc1-c1cn(Cc2csc(C)n2)c(=O)cc1C(=O)Nc1nnc(C#CC2CC2)s1] | 332 |
| 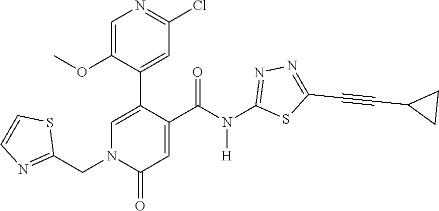 [SMILES: COc1cnc(Cl)cc1-c1cn(Cc2nccs2)c(=O)cc1C(=O)Nc1nnc(C#CC2CC2)s1] | 333 |
| 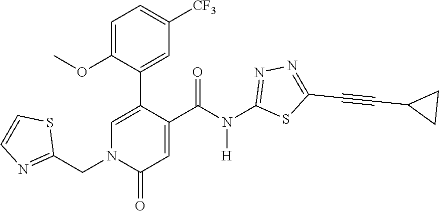 [SMILES: COc1ccc(C(F)(F)F)cc1-c1cn(Cc2nccs2)c(=O)cc1C(=O)Nc1nnc(C#CC2CC2)s1] | 334 |
| 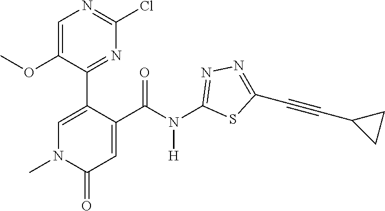 [SMILES: COc1cnc(Cl)nc1-c1cn(C)c(=O)cc1C(=O)Nc1nnc(C#CC2CC2)s1] | 335 |
| 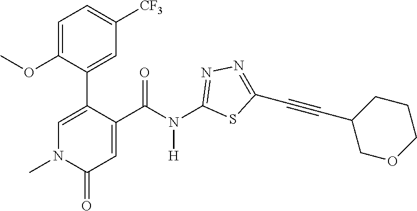 [SMILES: COc1ccc(C(F)(F)F)cc1-c1cn(C)c(=O)cc1C(=O)Nc1nnc(C#CC2CCCOC2)s1] | 336 |
| 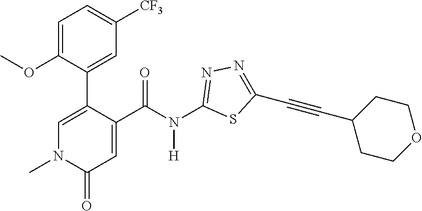 [SMILES: COc1ccc(C(F)(F)F)cc1-c1cn(C)c(=O)cc1C(=O)Nc1nnc(C#CC2CCOCC2)s1] | 337 |
| 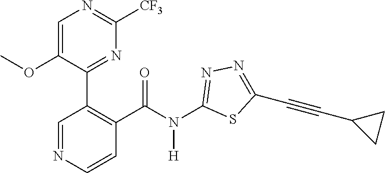 [SMILES: COc1cnc(C(F)(F)F)nc1-c1cnccc1C(=O)Nc1nnc(C#CC2CC2)s1] | 338 |
| 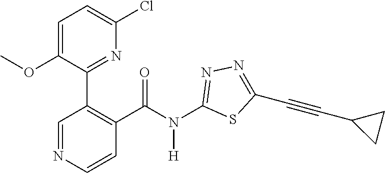 [SMILES: COc1ccc(Cl)nc1-c1cnccc1C(=O)Nc1nnc(C#CC2CC2)s1] | 339 |
| 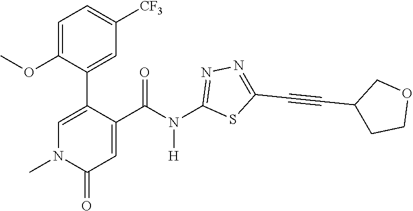 [SMILES: COc1ccc(C(F)(F)F)cc1-c1cn(C)c(=O)cc1C(=O)Nc1nnc(C#CC2CCOC2)s1] | 340 |
| 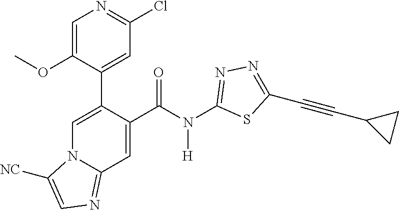 [SMILES: COc1cnc(Cl)cc1-c1cn2c(C#N)cnc2cc1C(=O)Nc1nnc(C#CC2CC2)s1] | 341 |
| 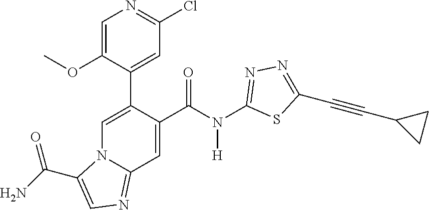 [SMILES: COc1cnc(Cl)cc1-c1cn2c(C(N)=O)cnc2cc1C(=O)Nc1nnc(C#CC2CC2)s1] | 342 |
| 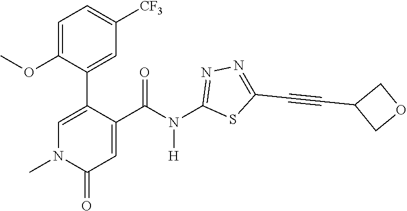 [SMILES: COc1ccc(C(F)(F)F)cc1-c1cn(C)c(=O)cc1C(=O)Nc1nnc(C#CC2COC2)s1] | 343 |
| 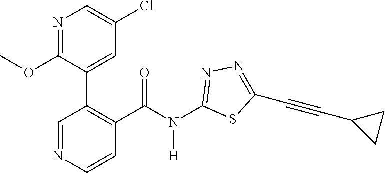 [SMILES: COc1ncc(Cl)cc1-c1cnccc1C(=O)Nc1nnc(C#CC2CC2)s1] | 344 |
| 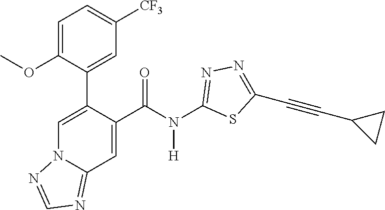 [SMILES: COc1ccc(C(F)(F)F)cc1-c1cn2ncnc2cc1C(=O)Nc1nnc(C#CC2CC2)s1] | 345 |
| 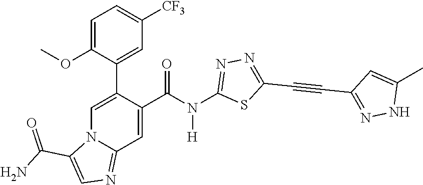 [SMILES: COc1ccc(C(F)(F)F)cc1-c1cn2c(C(N)=O)cnc2cc1C(=O)Nc1nnc(C#Cc2cc(C)[nH]n2)s1] | 346 |
| 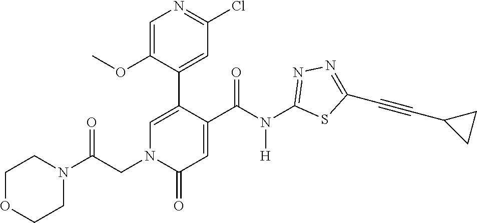 [SMILES: COc1cnc(Cl)cc1-c1cn(CC(=O)N2CCOCC2)c(=O)cc1C(=O)Nc1nnc(C#CC2CC2)s1] | 347 |
| 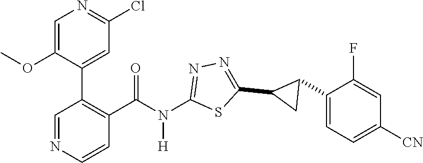 | 348 |
| 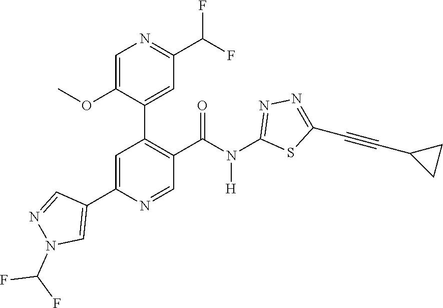 [SMILES: COc1cnc(C(F)F)cc1-c1cc(-c2cnn(C(F)F)c2)ncc1C(=O)Nc1nnc(C#CC2CC2)s1] | 349 |
| 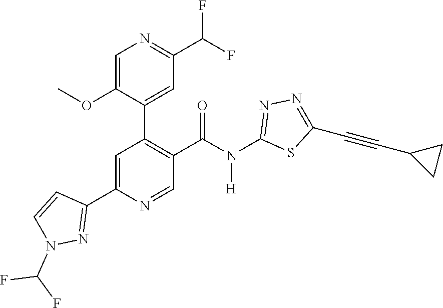 [SMILES: COc1cnc(C(F)F)cc1-c1cc(-c2ccn(C(F)F)n2)ncc1C(=O)Nc1nnc(C#CC2CC2)s1] | 350 |
| 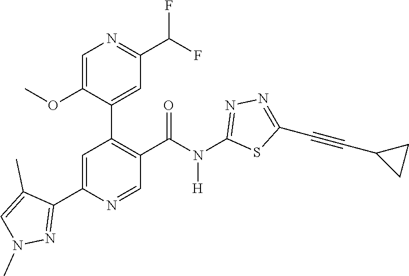 [SMILES: COc1cnc(C(F)F)cc1-c1cc(-c2nn(C)cc2C)ncc1C(=O)Nc1nnc(C#CC2CC2)s1] | 351 |
| 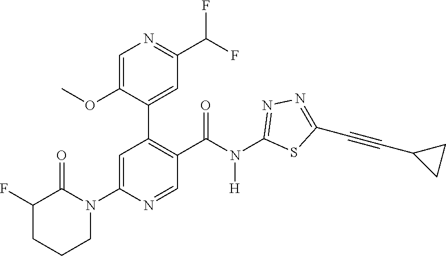 [SMILES: COc1cnc(C(F)F)cc1-c1cc(N2CCCC(F)C2=O)ncc1C(=O)Nc1nnc(C#CC2CC2)s1] | 352 |
| 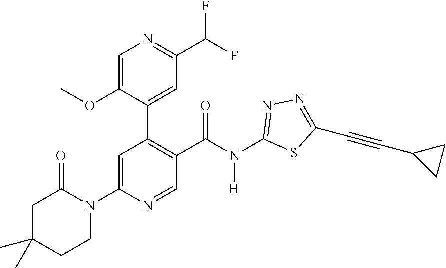 [SMILES: COc1cnc(C(F)F)cc1-c1cc(N2CCC(C)(C)CC2=O)ncc1C(=O)Nc1nnc(C#CC2CC2)s1] | 353 |
| 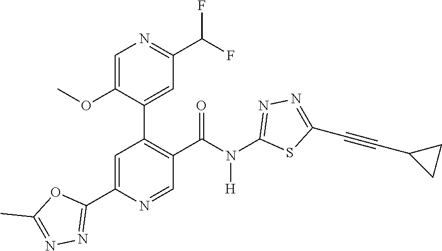 [SMILES: COc1cnc(C(F)F)cc1-c1cc(-c2nnc(C)o2)ncc1C(=O)Nc1nnc(C#CC2CC2)s1] | 354 |
| 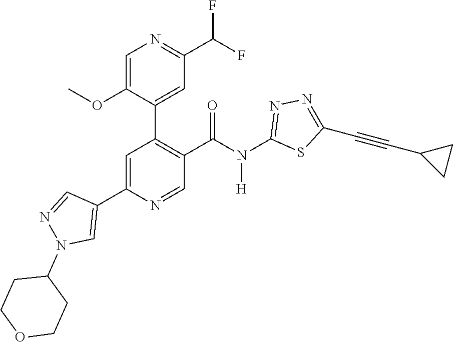 [SMILES: COc1cnc(C(F)F)cc1-c1cc(-c2cnn(C3CCOCC3)c2)ncc1C(=O)Nc1nnc(C#CC2CC2)s1] | 355 |
| 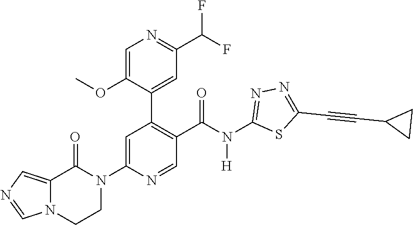 [SMILES: COc1cnc(C(F)F)cc1-c1cc(N2CCn3cncc3C2=O)ncc1C(=O)Nc1nnc(C#CC2CC2)s1] | 356 |
| 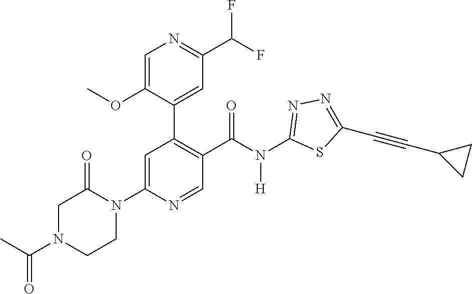 [SMILES: COc1cnc(C(F)F)cc1-c1cc(N2CCN(C(C)=O)CC2=O)ncc1C(=O)Nc1nnc(C#CC2CC2)s1] | 357 |
| 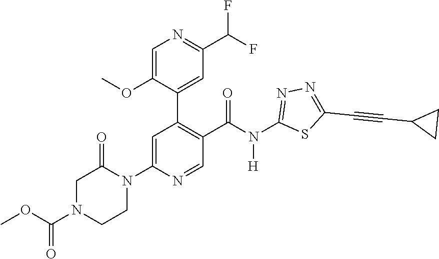 [SMILES: COC(=O)N1CCN(c2cc(-c3cc(C(F)F)ncc3OC)c(C(=O)Nc3nnc(C#CC4CC4)s3)cn2)C(=O)C1] | 358 |

In some embodiments, the compound of the invention may be a compound listed in Table 2 below or a pharmaceutically acceptable salt thereof.

TABLE 2

| | |
|---|---|
| 359 | 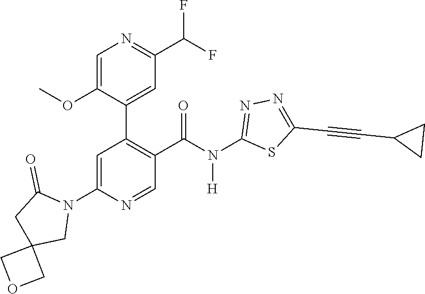 [SMILES: COc1cnc(C(F)F)cc1-c1cc(N2CC3(COC3)CC2=O)ncc1C(=O)Nc1nnc(C#CC2CC2)s1] |
| 360 | 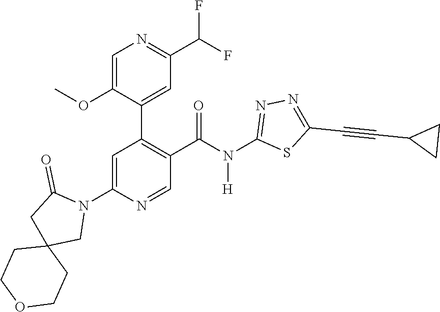 [SMILES: COc1cnc(C(F)F)cc1-c1cc(N2CC3(CCOCC3)CC2=O)ncc1C(=O)Nc1nnc(C#CC2CC2)s1] |
| 361 | 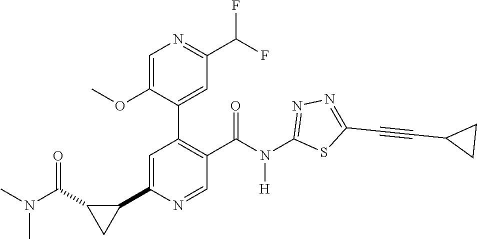 Racemic |
| 362 | 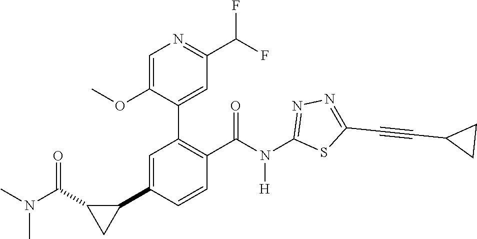 Racemic |
| 363 | 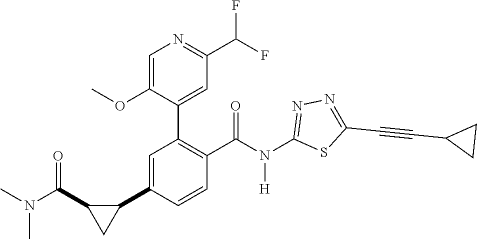 Racemic |
| 364 | 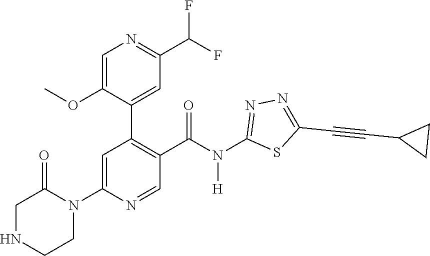 [SMILES: COc1cnc(C(F)F)cc1-c1cc(N2CCNCC2=O)ncc1C(=O)Nc1nnc(C#CC2CC2)s1] |
| 365 | 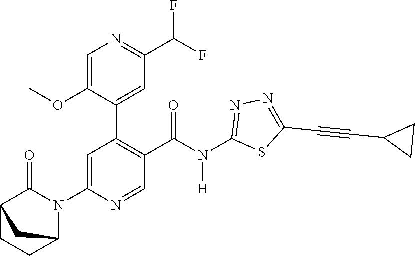 [SMILES: COc1cnc(C(F)F)cc1-c1cc(N2C(=O)[C@H]3CC[C@@H]2C3)ncc1C(=O)Nc1nnc(C#CC2CC2)s1] Chiral |
| 366 | 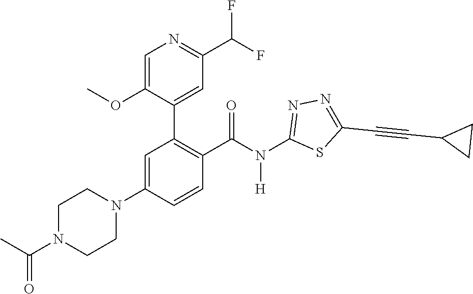 [SMILES: COc1cnc(C(F)F)cc1-c1cc(N2CCN(C(C)=O)CC2)ccc1C(=O)Nc1nnc(C#CC2CC2)s1] |
| 367 | 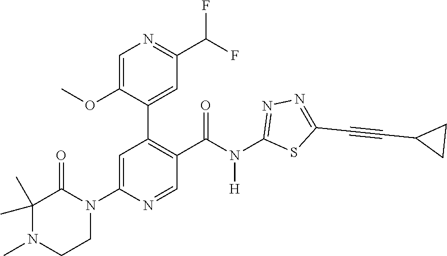 [SMILES: COc1cnc(C(F)F)cc1-c1cc(N2CCN(C)C(C)(C)C2=O)ncc1C(=O)Nc1nnc(C#CC2CC2)s1] |
| 368 | 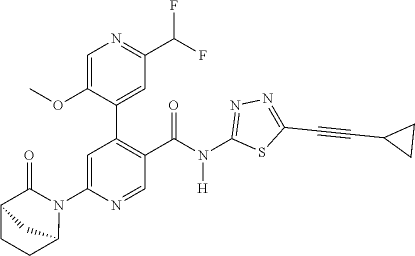 [SMILES: COc1cnc(C(F)F)cc1-c1cc(N2C(=O)[C@@H]3CC[C@H]2C3)ncc1C(=O)Nc1nnc(C#CC2CC2)s1] Chiral |
| 369 | 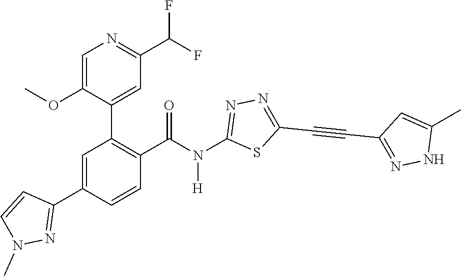 [SMILES: COc1cnc(C(F)F)cc1-c1cc(-c2ccn(C)n2)ccc1C(=O)Nc1nnc(C#Cc2cc(C)[nH]n2)s1] |
| 370 | 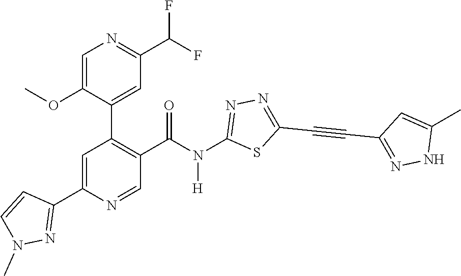 [SMILES: COc1cnc(C(F)F)cc1-c1cc(-c2ccn(C)n2)ncc1C(=O)Nc1nnc(C#Cc2cc(C)[nH]n2)s1] |
| 371 | 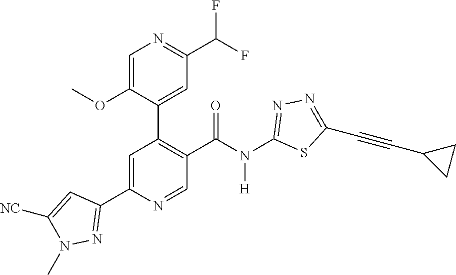 [SMILES: COc1cnc(C(F)F)cc1-c1cc(-c2cc(C#N)n(C)n2)ncc1C(=O)Nc1nnc(C#CC2CC2)s1] |
| 372 | 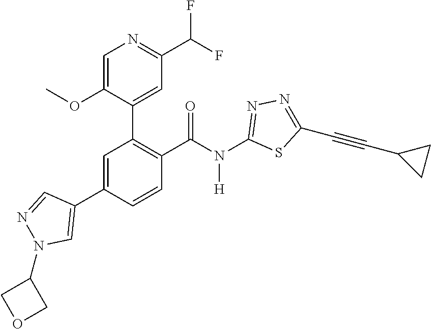 [SMILES: COc1cnc(C(F)F)cc1-c1cc(-c2cnn(C3COC3)c2)ccc1C(=O)Nc1nnc(C#CC2CC2)s1] |
| 373 | 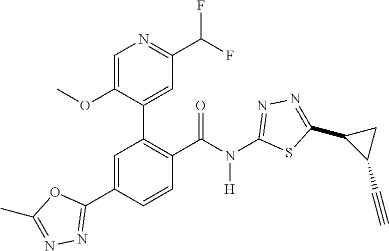 Racemic |
| 374 | 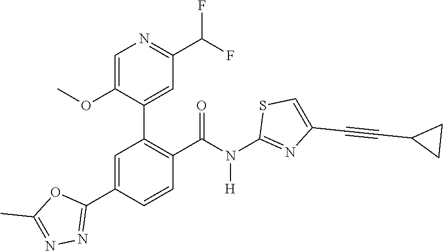 [SMILES: COc1cnc(C(F)F)cc1-c1cc(-c2nnc(C)o2)ccc1C(=O)Nc1nc(C#CC2CC2)cs1] |
| 375 | 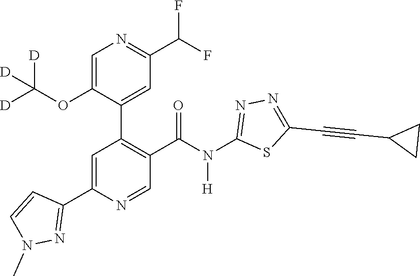 [SMILES: [2H]C([2H])([2H])Oc1cnc(C(F)F)cc1-c1cc(-c2ccn(C)n2)ncc1C(=O)Nc1nnc(C#CC2CC2)s1] |
| 376 | 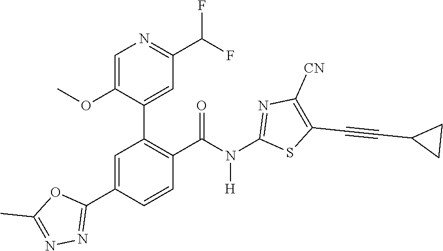 [SMILES: COc1cnc(C(F)F)cc1-c1cc(-c2nnc(C)o2)ccc1C(=O)Nc1nc(C#N)c(C#CC2CC2)s1] |
| 377 | 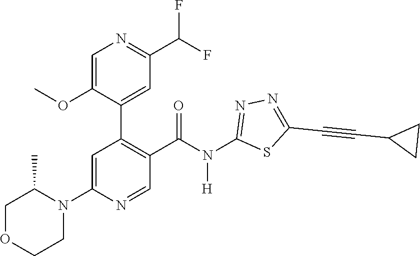 [SMILES: COc1cnc(C(F)F)cc1-c1cc(N2CCOC[C@@H]2C)ncc1C(=O)Nc1nnc(C#CC2CC2)s1] Chiral |
| 378 | 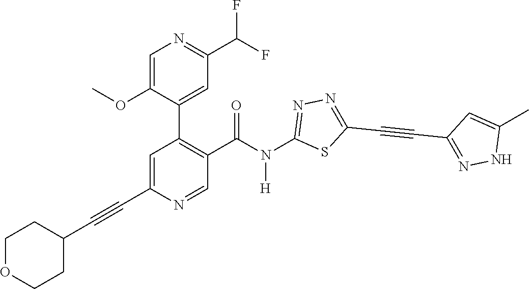 [SMILES: COc1cnc(C(F)F)cc1-c1cc(C#CC2CCOCC2)ncc1C(=O)Nc1nnc(C#Cc2cc(C)[nH]n2)s1] |
| 379 | 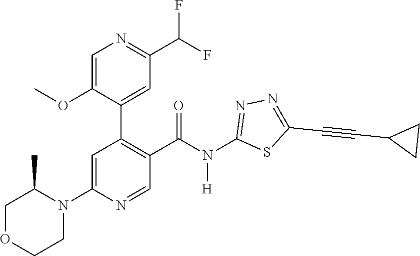 [SMILES: COc1cnc(C(F)F)cc1-c1cc(N2CCOC[C@H]2C)ncc1C(=O)Nc1nnc(C#CC2CC2)s1] Chiral |
| 380 | 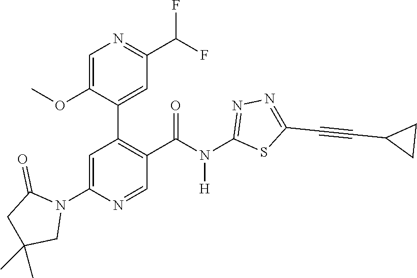 [SMILES: COc1cnc(C(F)F)cc1-c1cc(N2CC(C)(C)CC2=O)ncc1C(=O)Nc1nnc(C#CC2CC2)s1] |
| 381 | 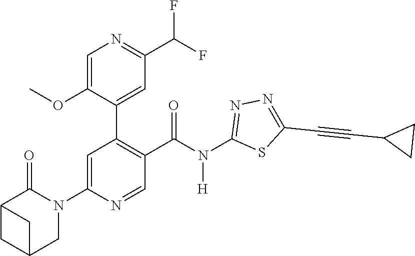 [SMILES: COc1cnc(C(F)F)cc1-c1cc(N2CC3CC(C3)C2=O)ncc1C(=O)Nc1nnc(C#CC2CC2)s1] Racemic |
| 382 | 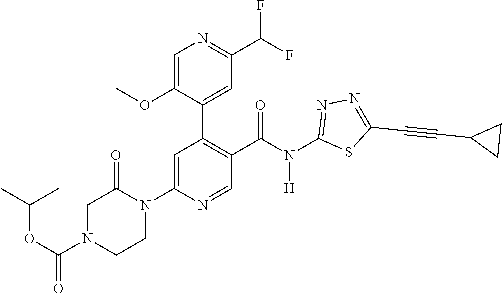 [SMILES: COc1cnc(C(F)F)cc1-c1cc(N2CCN(C(=O)OC(C)C)CC2=O)ncc1C(=O)Nc1nnc(C#CC2CC2)s1] |
| 383 | 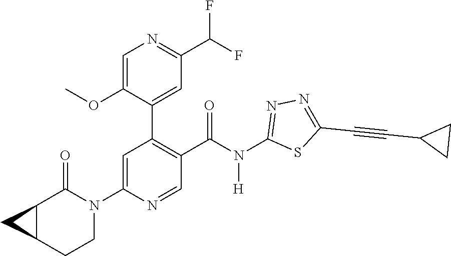 [SMILES: COc1cnc(C(F)F)cc1-c1cc(N2CC[C@H]3C[C@H]3C2=O)ncc1C(=O)Nc1nnc(C#CC2CC2)s1] Chiral |
| 384 | 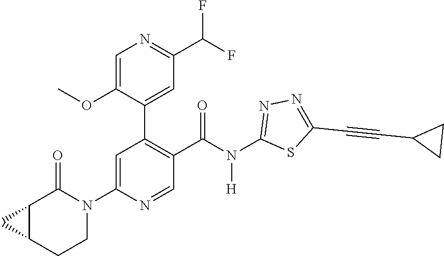 [SMILES: COc1cnc(C(F)F)cc1-c1cc(N2CC[C@@H]3C[C@@H]3C2=O)ncc1C(=O)Nc1nnc(C#CC2CC2)s1] Chiral |
| 385 | 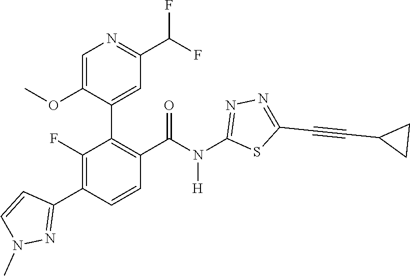 [SMILES: COc1cnc(C(F)F)cc1-c1c(C(=O)Nc2nnc(C#CC3CC3)s2)ccc(-c2ccn(C)n2)c1F] |
| 386 | 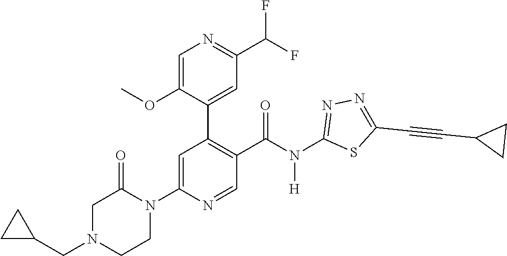 [SMILES: COc1cnc(C(F)F)cc1-c1cc(N2CCN(CC3CC3)CC2=O)ncc1C(=O)Nc1nnc(C#CC2CC2)s1] |
| 387 | 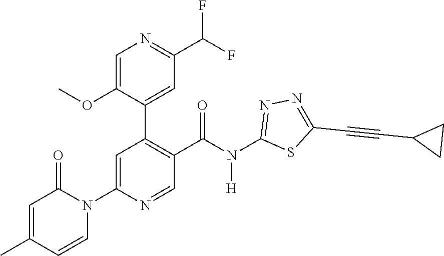 [SMILES: COc1cnc(C(F)F)cc1-c1cc(-n2ccc(C)cc2=O)ncc1C(=O)Nc1nnc(C#CC2CC2)s1] |
| 388 | 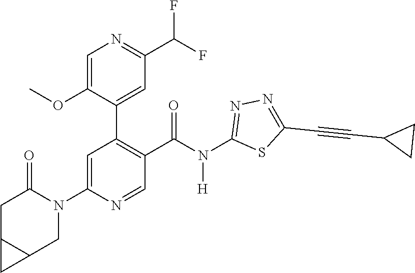 [SMILES: COc1cnc(C(F)F)cc1-c1cc(N2CC3CC3CC2=O)ncc1C(=O)Nc1nnc(C#CC2CC2)s1] Racemic |
| 389 | 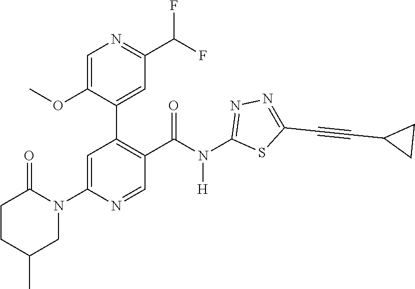 [SMILES: COc1cnc(C(F)F)cc1-c1cc(N2CC(C)CCC2=O)ncc1C(=O)Nc1nnc(C#CC2CC2)s1] Racemic |
| 390 | 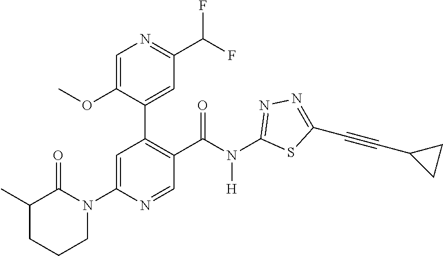 [SMILES: COc1cnc(C(F)F)cc1-c1cc(N2CCCC(C)C2=O)ncc1C(=O)Nc1nnc(C#CC2CC2)s1] |
| 391 | 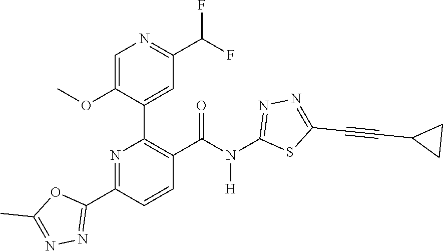 [SMILES: COc1cnc(C(F)F)cc1-c1nc(-c2nnc(C)o2)ccc1C(=O)Nc1nnc(C#CC2CC2)s1] |
| 392 | 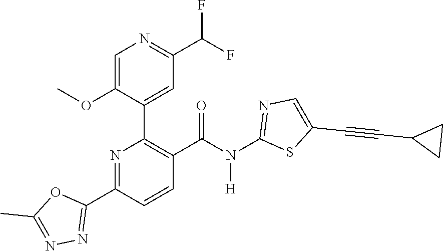 [SMILES: COc1cnc(C(F)F)cc1-c1nc(-c2nnc(C)o2)ccc1C(=O)Nc1ncc(C#CC2CC2)s1] |
| 393 | 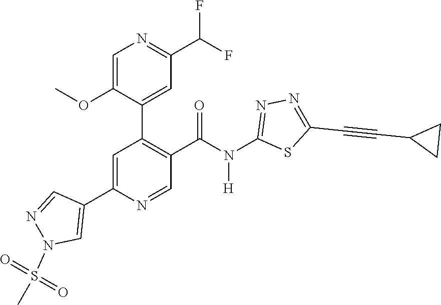 [SMILES: COc1cnc(C(F)F)cc1-c1cc(-c2cnn(S(C)(=O)=O)c2)ncc1C(=O)Nc1nnc(C#CC2CC2)s1] |
| 394 | 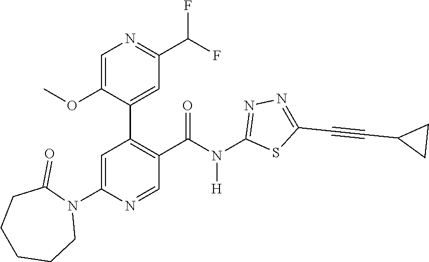 [SMILES: COc1cnc(C(F)F)cc1-c1cc(N2CCCCCC2=O)ncc1C(=O)Nc1nnc(C#CC2CC2)s1] |
| 395 | 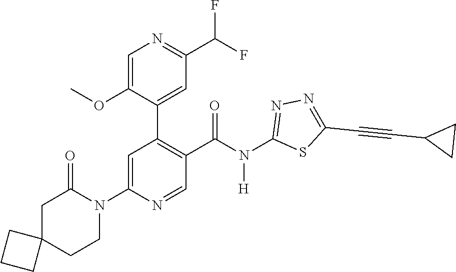 [SMILES: COc1cnc(C(F)F)cc1-c1cc(N2CCC3(CCC3)CC2=O)ncc1C(=O)Nc1nnc(C#CC2CC2)s1] |
| 396 | 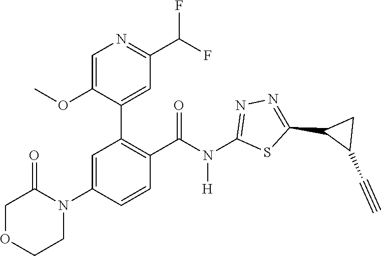 Chiral |
| 397 | 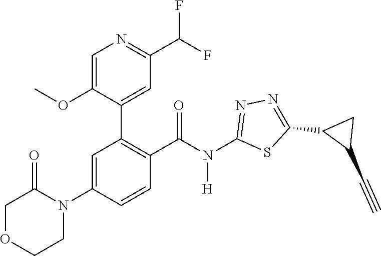 Chiral |
| 398 | 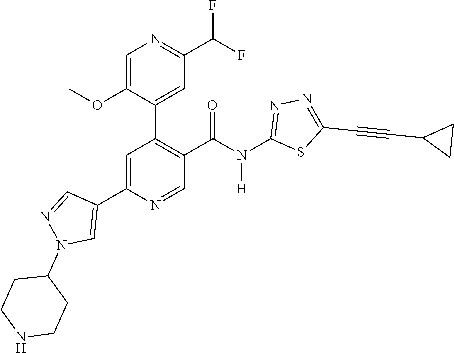 [SMILES: COc1cnc(C(F)F)cc1-c1cc(-c2cnn(C3CCNCC3)c2)ncc1C(=O)Nc1nnc(C#CC2CC2)s1] |
| 399 | 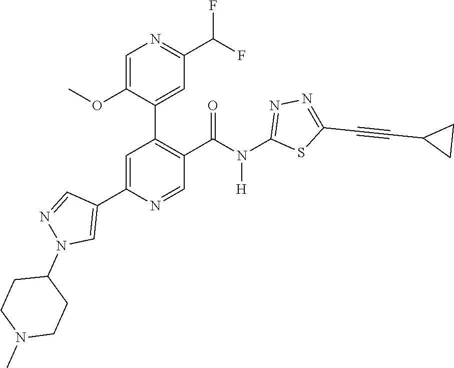 [SMILES: COc1cnc(C(F)F)cc1-c1cc(-c2cnn(C3CCN(C)CC3)c2)ncc1C(=O)Nc1nnc(C#CC2CC2)s1] |
| 400 | 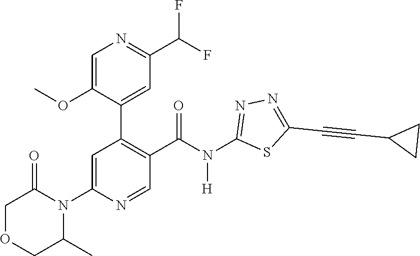 [SMILES: COc1cnc(C(F)F)cc1-c1cc(N2C(=O)COCC2C)ncc1C(=O)Nc1nnc(C#CC2CC2)s1] |
| 401 | 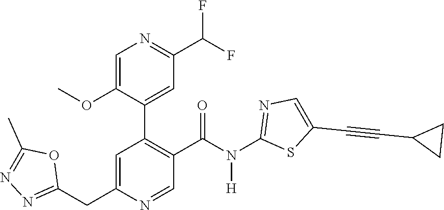 [SMILES: COc1cnc(C(F)F)cc1-c1cc(Cc2nnc(C)o2)ncc1C(=O)Nc1ncc(C#CC2CC2)s1] |
| 402 | 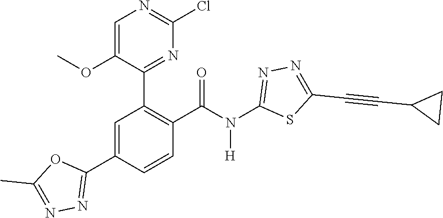 [SMILES: COc1cnc(Cl)nc1-c1cc(-c2nnc(C)o2)ccc1C(=O)Nc1nnc(C#CC2CC2)s1] |
| 403 | 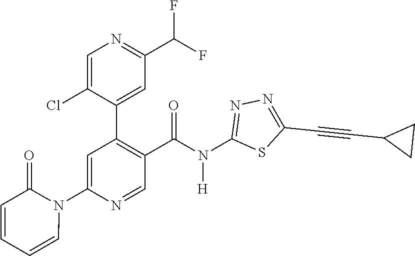 [SMILES: O=C(Nc1nnc(C#CC2CC2)s1)c1cnc(-n2ccccc2=O)cc1-c1cc(C(F)F)ncc1Cl] |
| 404 | 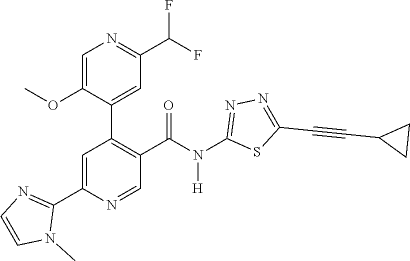 [SMILES: COc1cnc(C(F)F)cc1-c1cc(-c2nccn2C)ncc1C(=O)Nc1nnc(C#CC2CC2)s1] |
| 405 | 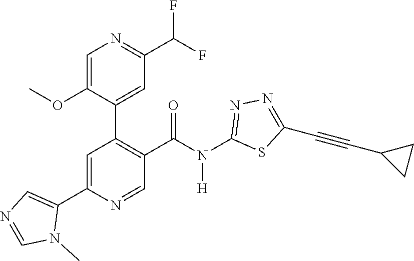 [SMILES: COc1cnc(C(F)F)cc1-c1cc(-c2cncn2C)ncc1C(=O)Nc1nnc(C#CC2CC2)s1] |
| 406 | 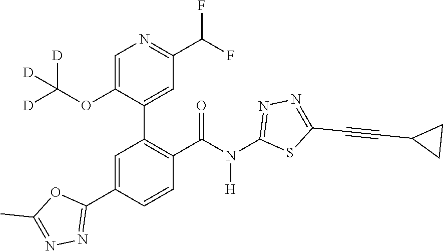 [SMILES: [2H]C([2H])([2H])Oc1cnc(C(F)F)cc1-c1cc(-c2nnc(C)o2)ccc1C(=O)Nc1nnc(C#CC2CC2)s1] |
| 407 | 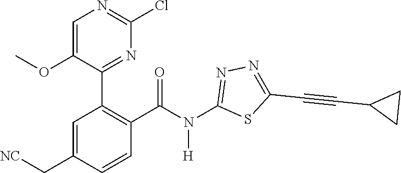 [SMILES: COc1cnc(Cl)nc1-c1cc(CC#N)ccc1C(=O)Nc1nnc(C#CC2CC2)s1] |
| 408 | 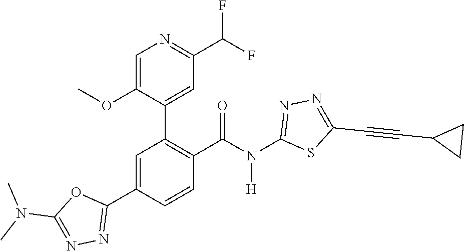 [SMILES: COc1cnc(C(F)F)cc1-c1cc(-c2nnc(N(C)C)o2)ccc1C(=O)Nc1nnc(C#CC2CC2)s1] |
| 409 | 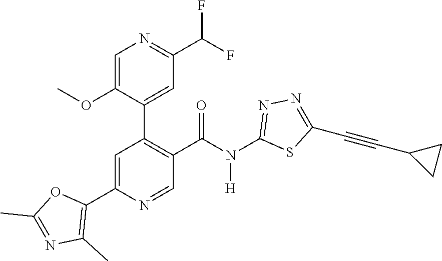 [SMILES: COc1cnc(C(F)F)cc1-c1cc(-c2oc(C)nc2C)ncc1C(=O)Nc1nnc(C#CC2CC2)s1] |
| 410 | 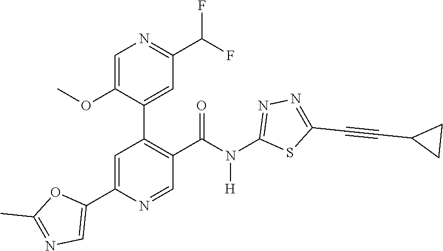 [SMILES: COc1cnc(C(F)F)cc1-c1cc(-c2cnc(C)o2)ncc1C(=O)Nc1nnc(C#CC2CC2)s1] |
| 411 | 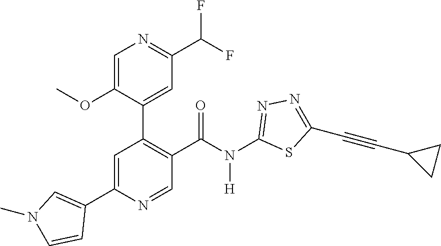 [SMILES: COc1cnc(C(F)F)cc1-c1cc(-c2ccn(C)c2)ncc1C(=O)Nc1nnc(C#CC2CC2)s1] |
| 412 | 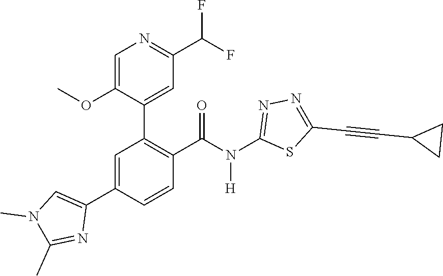 [SMILES: COc1cnc(C(F)F)cc1-c1cc(-c2cn(C)c(C)n2)ccc1C(=O)Nc1nnc(C#CC2CC2)s1] |
| 413 | 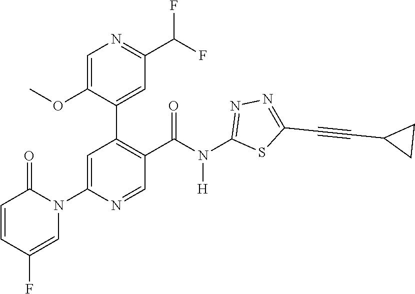 [SMILES: COc1cnc(C(F)F)cc1-c1cc(-n2cc(F)ccc2=O)ncc1C(=O)Nc1nnc(C#CC2CC2)s1] |
| 414 | 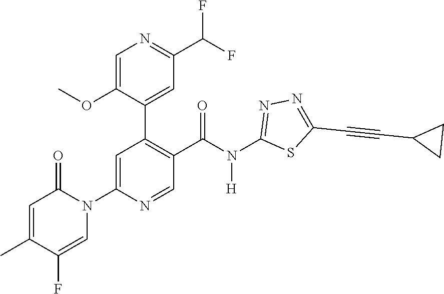 [SMILES: COc1cnc(C(F)F)cc1-c1cc(-n2cc(F)c(C)cc2=O)ncc1C(=O)Nc1nnc(C#CC2CC2)s1] |
| 415 | 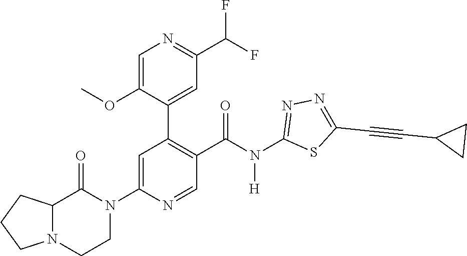 [SMILES: COc1cnc(C(F)F)cc1-c1cc(N2CCN3CCCC3C2=O)ncc1C(=O)Nc1nnc(C#CC2CC2)s1] |
| 416 | 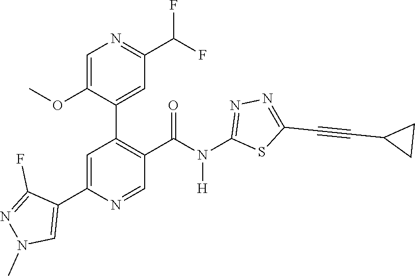 [SMILES: COc1cnc(C(F)F)cc1-c1cc(-c2cn(C)nc2F)ncc1C(=O)Nc1nnc(C#CC2CC2)s1] |
| 417 | 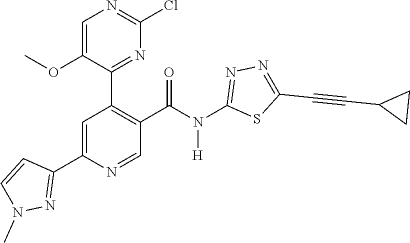 [SMILES: COc1cnc(Cl)nc1-c1cc(-c2ccn(C)n2)ncc1C(=O)Nc1nnc(C#CC2CC2)s1] |
| 418 | 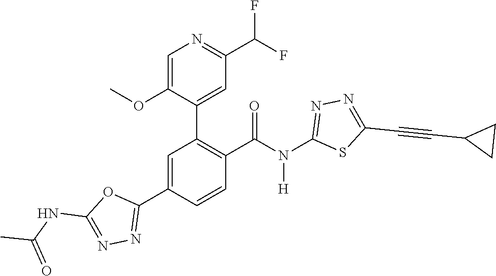 [SMILES: COc1cnc(C(F)F)cc1-c1cc(-c2nnc(NC(C)=O)o2)ccc1C(=O)Nc1nnc(C#CC2CC2)s1] |
| 419 | 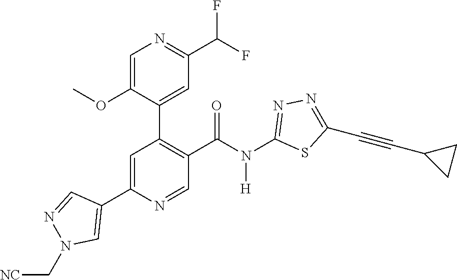 [SMILES: COc1cnc(C(F)F)cc1-c1cc(-c2cnn(CC#N)c2)ncc1C(=O)Nc1nnc(C#CC2CC2)s1] |
| 420 | 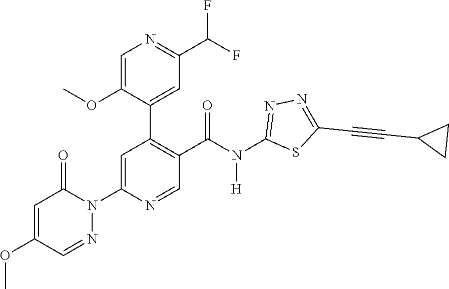 [SMILES: COc1cnn(-c2cc(-c3cc(C(F)F)ncc3OC)c(C(=O)Nc3nnc(C#CC4CC4)s3)cn2)c(=O)c1] |
| 421 | 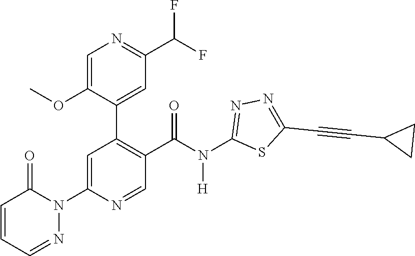 [SMILES: COc1cnc(C(F)F)cc1-c1cc(-n2ncccc2=O)ncc1C(=O)Nc1nnc(C#CC2CC2)s1] |
| 422 | 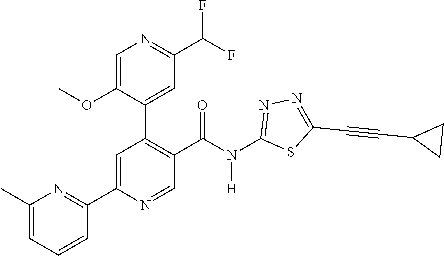 [SMILES: COc1cnc(C(F)F)cc1-c1cc(-c2cccc(C)n2)ncc1C(=O)Nc1nnc(C#CC2CC2)s1] |
| 423 | 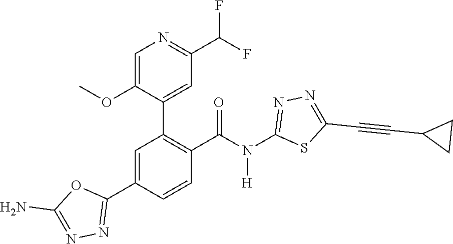 [SMILES: COc1cnc(C(F)F)cc1-c1cc(-c2nnc(N)o2)ccc1C(=O)Nc1nnc(C#CC2CC2)s1] |
| 424 | 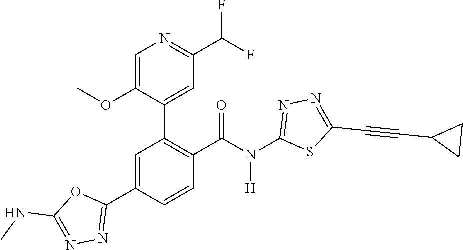 [SMILES: CNc1nnc(-c2ccc(C(=O)Nc3nnc(C#CC4CC4)s3)c(-c3cc(C(F)F)ncc3OC)c2)o1] |
| 425 | 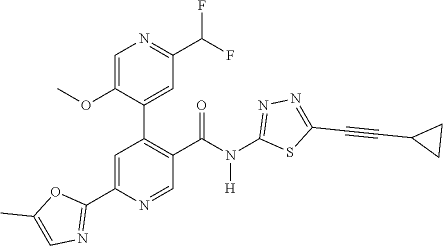 [SMILES: COc1cnc(C(F)F)cc1-c1cc(-c2ncc(C)o2)ncc1C(=O)Nc1nnc(C#CC2CC2)s1] |
| 426 | 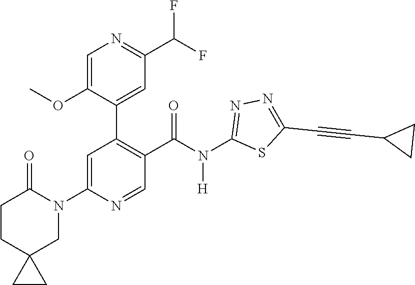 [SMILES: COc1cnc(C(F)F)cc1-c1cc(N2CC3(CCC2=O)CC3)ncc1C(=O)Nc1nnc(C#CC2CC2)s1] |
| 427 | 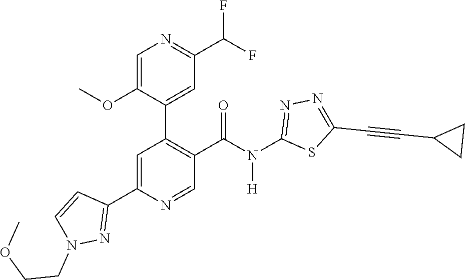 [SMILES: COCCn1ccc(-c2cc(-c3cc(C(F)F)ncc3OC)c(C(=O)Nc3nnc(C#CC4CC4)s3)cn2)n1] |
| 428 | 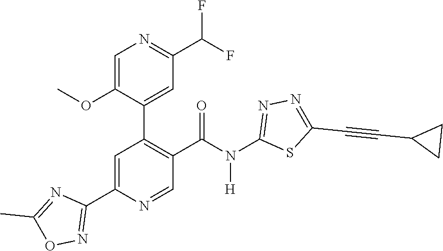 [SMILES: COc1cnc(C(F)F)cc1-c1cc(-c2noc(C)n2)ncc1C(=O)Nc1nnc(C#CC2CC2)s1] |
| 429 | 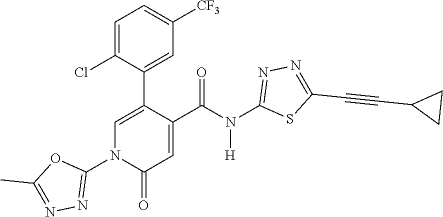 [SMILES: Cc1nnc(-n2cc(-c3cc(C(F)(F)F)ccc3Cl)c(C(=O)Nc3nnc(C#CC4CC4)s3)cc2=O)o1] |
| 430 | 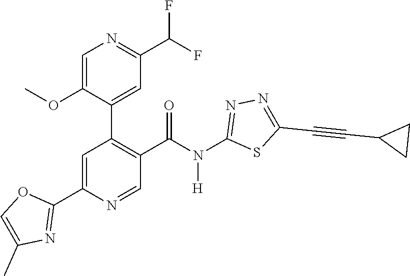 [SMILES: COc1cnc(C(F)F)cc1-c1cc(-c2nc(C)co2)ncc1C(=O)Nc1nnc(C#CC2CC2)s1] |
| 431 | 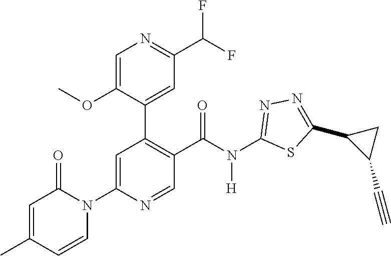 |
| 432 | 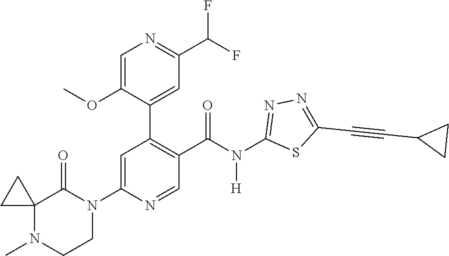 [SMILES: COc1cnc(C(F)F)cc1-c1cc(N2CCN(C)C3(CC3)C2=O)ncc1C(=O)Nc1nnc(C#CC2CC2)s1] |
| 433 | 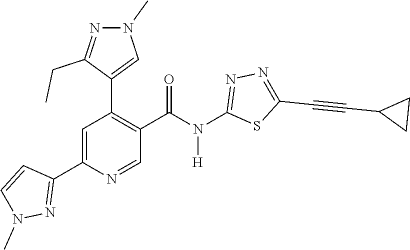 [SMILES: CCc1nn(C)cc1-c1cc(-c2ccn(C)n2)ncc1C(=O)Nc1nnc(C#CC2CC2)s1] |
| 434 | 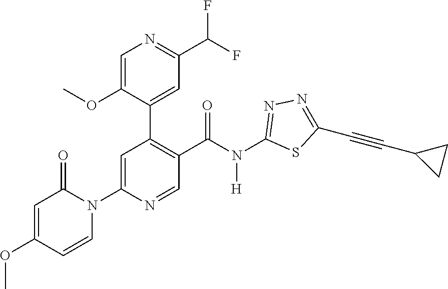 [SMILES: COc1ccn(-c2cc(-c3cc(C(F)F)ncc3OC)c(C(=O)Nc3nnc(C#CC4CC4)s3)cn2)c(=O)c1] |
| 435 | 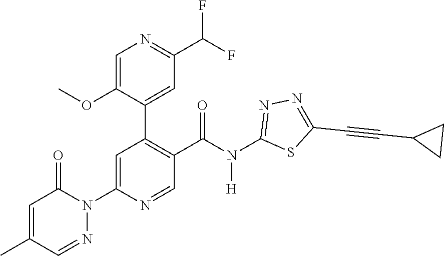 [SMILES: COc1cnc(C(F)F)cc1-c1cc(-n2ncc(C)cc2=O)ncc1C(=O)Nc1nnc(C#CC2CC2)s1] |
| 436 | 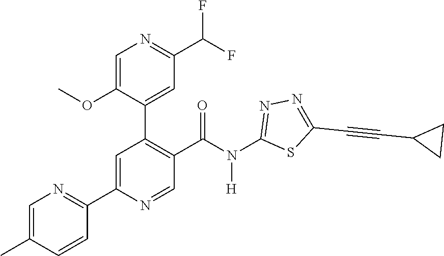 [SMILES: COc1cnc(C(F)F)cc1-c1cc(-c2ccc(C)cn2)ncc1C(=O)Nc1nnc(C#CC2CC2)s1] |
| 437 | 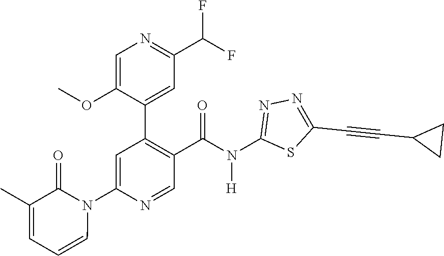 [SMILES: COc1cnc(C(F)F)cc1-c1cc(-n2cccc(C)c2=O)ncc1C(=O)Nc1nnc(C#CC2CC2)s1] |
| 438 | 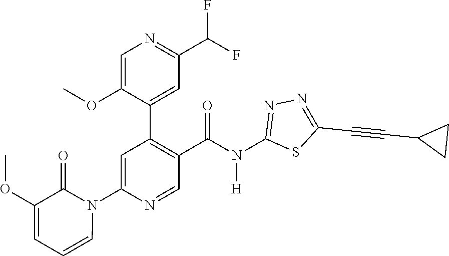 [SMILES: COc1cnc(C(F)F)cc1-c1cc(-n2cccc(OC)c2=O)ncc1C(=O)Nc1nnc(C#CC2CC2)s1] |
| 439 | 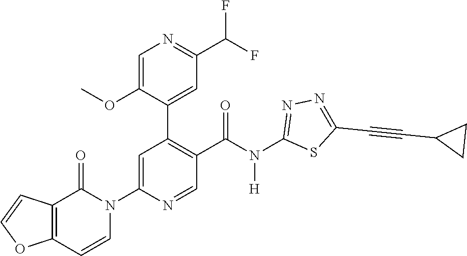 [SMILES: COc1cnc(C(F)F)cc1-c1cc(-n2ccc3occc3c2=O)ncc1C(=O)Nc1nnc(C#CC2CC2)s1] |
| 440 | 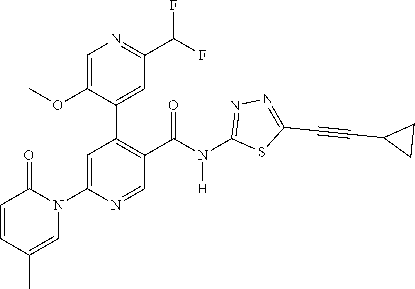 [SMILES: COc1cnc(C(F)F)cc1-c1cc(-n2cc(C)ccc2=O)ncc1C(=O)Nc1nnc(C#CC2CC2)s1] |
| 441 | 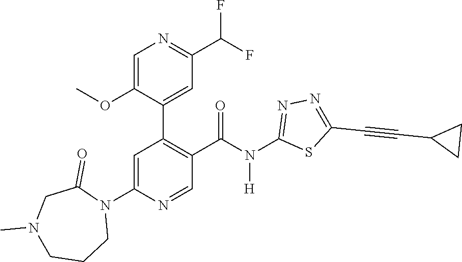 [SMILES: COc1cnc(C(F)F)cc1-c1cc(N2CCCN(C)CC2=O)ncc1C(=O)Nc1nnc(C#CC2CC2)s1] |
| 442 | 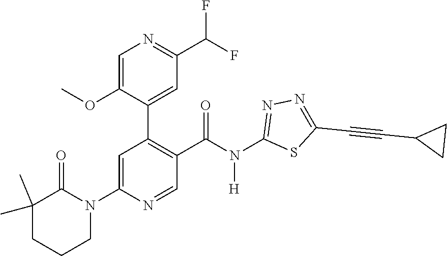 [SMILES: COc1cnc(C(F)F)cc1-c1cc(N2CCCC(C)(C)C2=O)ncc1C(=O)Nc1nnc(C#CC2CC2)s1] |
| 443 | 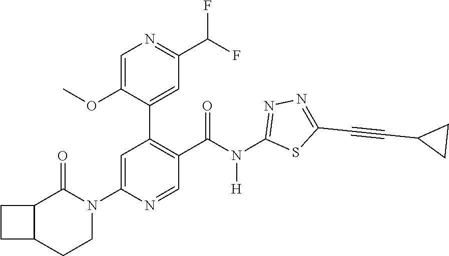 [SMILES: COc1cnc(C(F)F)cc1-c1cc(N2CCC3CCC3C2=O)ncc1C(=O)Nc1nnc(C#CC2CC2)s1] Racemic |
| 444 | 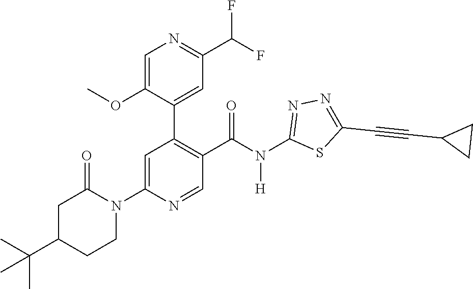 [SMILES: COc1cnc(C(F)F)cc1-c1cc(N2CCC(C(C)(C)C)CC2=O)ncc1C(=O)Nc1nnc(C#CC2CC2)s1] Racemic |
| 445 | 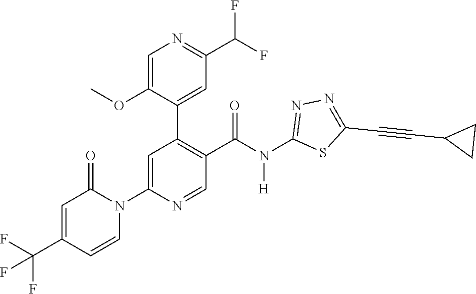 [SMILES: COc1cnc(C(F)F)cc1-c1cc(-n2ccc(C(F)(F)F)cc2=O)ncc1C(=O)Nc1nnc(C#CC2CC2)s1] |
| 446 | 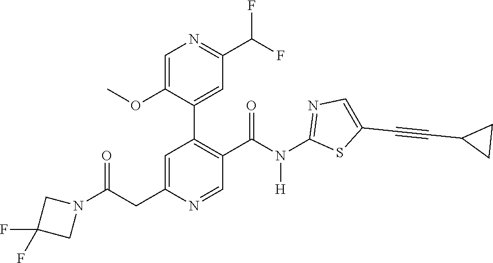 [SMILES: COc1cnc(C(F)F)cc1-c1cc(CC(=O)N2CC(F)(F)C2)ncc1C(=O)Nc1ncc(C#CC2CC2)s1] |
| 447 | 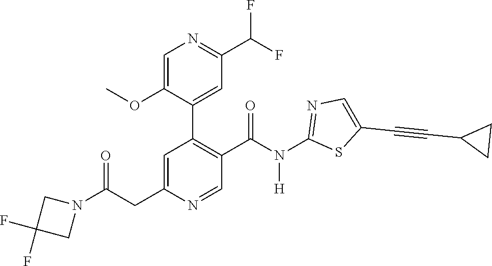 [SMILES: COc1cnc(C(F)F)cc1-c1cc(CC(=O)N2CC(F)(F)C2)ncc1C(=O)Nc1ncc(C#CC2CC2)s1] |
| 448 | 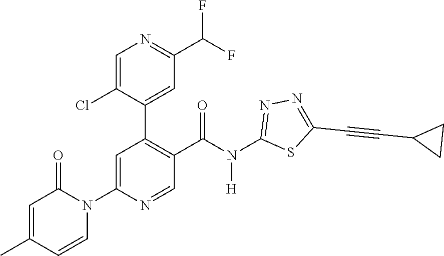 [SMILES: Cc1ccn(-c2cc(-c3cc(C(F)F)ncc3Cl)c(C(=O)Nc3nnc(C#CC4CC4)s3)cn2)c(=O)c1] |
| 449 | 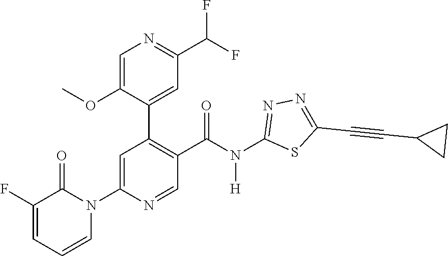 [SMILES: COc1cnc(C(F)F)cc1-c1cc(-n2cccc(F)c2=O)ncc1C(=O)Nc1nnc(C#CC2CC2)s1] |
| 450 | 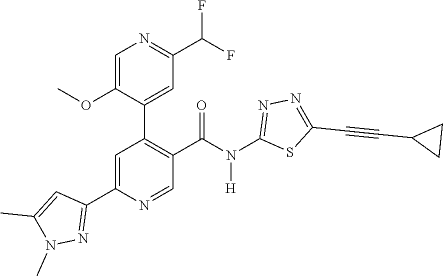 [SMILES: COc1cnc(C(F)F)cc1-c1cc(-c2cc(C)n(C)n2)ncc1C(=O)Nc1nnc(C#CC2CC2)s1] |
| 451 | 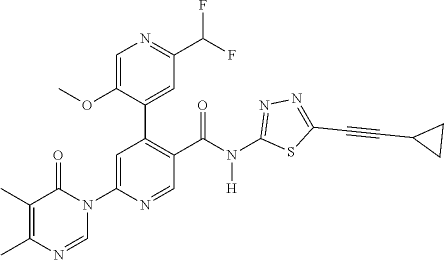 [SMILES: COc1cnc(C(F)F)cc1-c1cc(-n2cnc(C)c(C)c2=O)ncc1C(=O)Nc1nnc(C#CC2CC2)s1] |
| 452 | 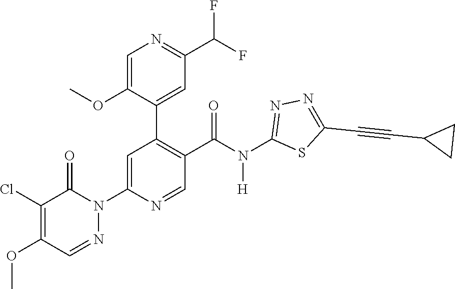 [SMILES: COc1cnc(C(F)F)cc1-c1cc(-n2ncc(OC)c(Cl)c2=O)ncc1C(=O)Nc1nnc(C#CC2CC2)s1] |
| 453 | 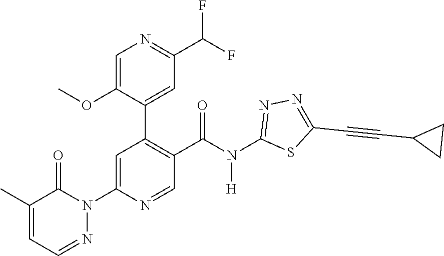 [SMILES: COc1cnc(C(F)F)cc1-c1cc(-n2nccc(C)c2=O)ncc1C(=O)Nc1nnc(C#CC2CC2)s1] |
| 454 | 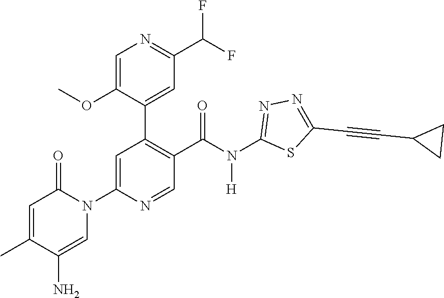 [SMILES: COc1cnc(C(F)F)cc1-c1cc(-n2cc(N)c(C)cc2=O)ncc1C(=O)Nc1nnc(C#CC2CC2)s1] |
| 455 | 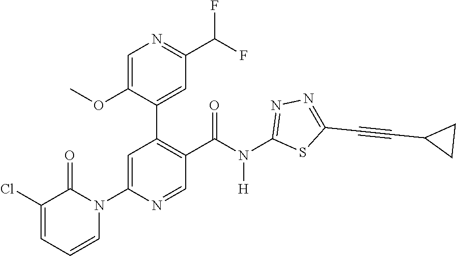 [SMILES: COc1cnc(C(F)F)cc1-c1cc(-n2cccc(Cl)c2=O)ncc1C(=O)Nc1nnc(C#CC2CC2)s1] |
| 456 | 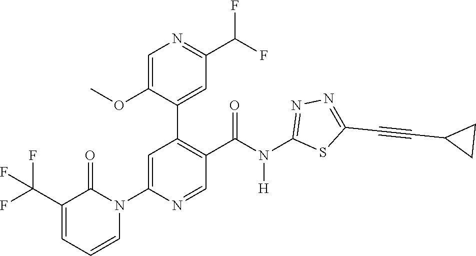 [SMILES: COc1cnc(C(F)F)cc1-c1cc(-n2cccc(C(F)(F)F)c2=O)ncc1C(=O)Nc1nnc(C#CC2CC2)s1] |
| 457 | 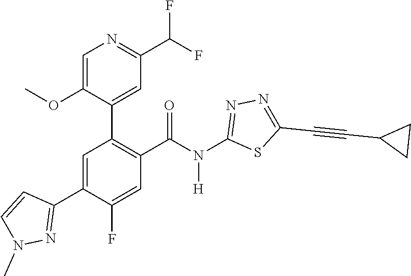 [SMILES: COc1cnc(C(F)F)cc1-c1cc(-c2ccn(C)n2)c(F)cc1C(=O)Nc1nnc(C#CC2CC2)s1] |
| 458 | 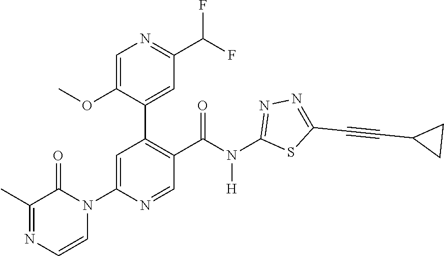 [SMILES: COc1cnc(C(F)F)cc1-c1cc(-n2ccnc(C)c2=O)ncc1C(=O)Nc1nnc(C#CC2CC2)s1] |
| 459 | 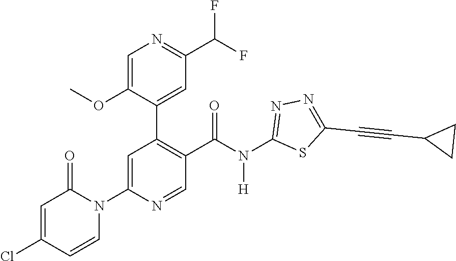 [SMILES: COc1cnc(C(F)F)cc1-c1cc(-n2ccc(Cl)cc2=O)ncc1C(=O)Nc1nnc(C#CC2CC2)s1] |
| 460 | 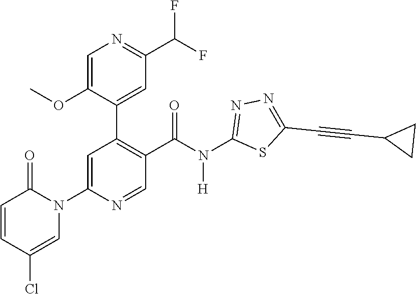 [SMILES: COc1cnc(C(F)F)cc1-c1cc(-n2cc(Cl)ccc2=O)ncc1C(=O)Nc1nnc(C#CC2CC2)s1] |
| 461 | 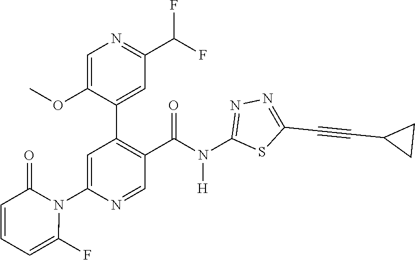 [SMILES: COc1cnc(C(F)F)cc1-c1cc(-n2c(F)cccc2=O)ncc1C(=O)Nc1nnc(C#CC2CC2)s1] |
| 462 | 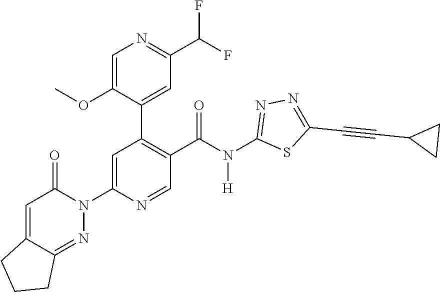 [SMILES: COc1cnc(C(F)F)cc1-c1cc(-n2nc3c(cc2=O)CCC3)ncc1C(=O)Nc1nnc(C#CC2CC2)s1] |
| 463 | 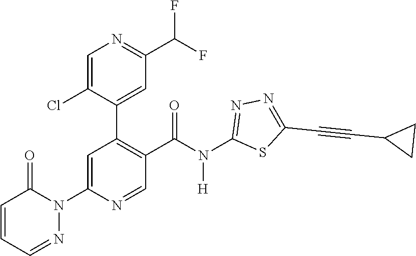 [SMILES: O=C(Nc1nnc(C#CC2CC2)s1)c1cnc(-n2ncccc2=O)cc1-c1cc(C(F)F)ncc1Cl] |
| 464 | 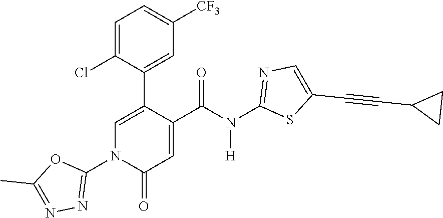 [SMILES: Cc1nnc(-n2cc(-c3cc(C(F)(F)F)ccc3Cl)c(C(=O)Nc3ncc(C#CC4CC4)s3)cc2=O)o1] |
| 465 | 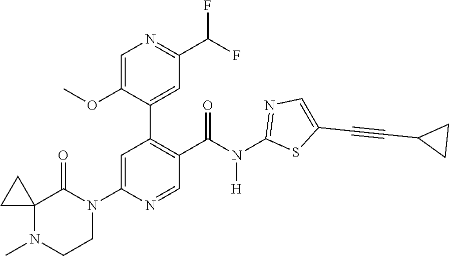 [SMILES: COc1cnc(C(F)F)cc1-c1cc(N2CCN(C)C3(CC3)C2=O)ncc1C(=O)Nc1ncc(C#CC2CC2)s1] |
| 466 | 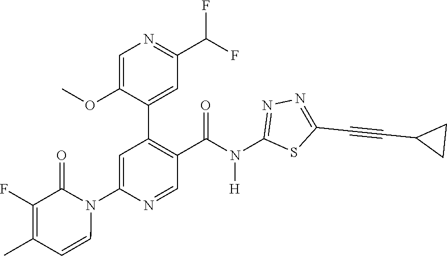 [SMILES: COc1cnc(C(F)F)cc1-c1cc(-n2ccc(C)c(F)c2=O)ncc1C(=O)Nc1nnc(C#CC2CC2)s1] |
| 467 | 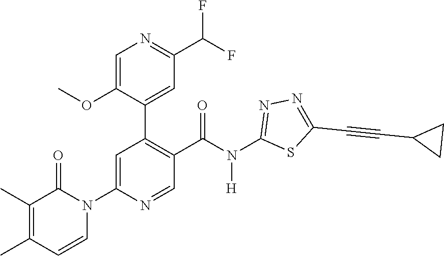 [SMILES: COc1cnc(C(F)F)cc1-c1cc(-n2ccc(C)c(C)c2=O)ncc1C(=O)Nc1nnc(C#CC2CC2)s1] |
| 468 | 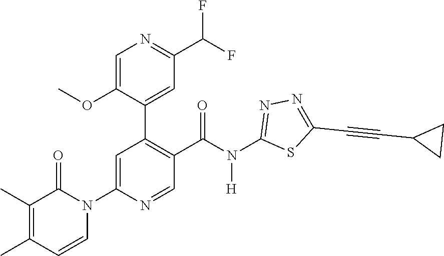 [SMILES: COc1cnc(C(F)F)cc1-c1cc(-n2ccc(C)c(C)c2=O)ncc1C(=O)Nc1nnc(C#CC2CC2)s1] |
| 469 | 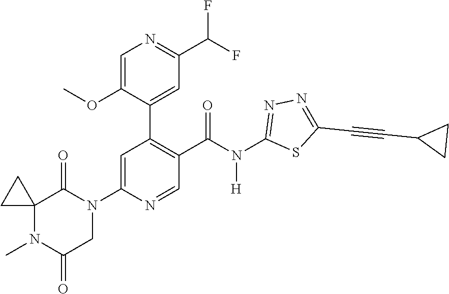 [SMILES: COc1cnc(C(F)F)cc1-c1cc(N2CC(=O)N(C)C3(CC3)C2=O)ncc1C(=O)Nc1nnc(C#CC2CC2)s1] |
| 470 | 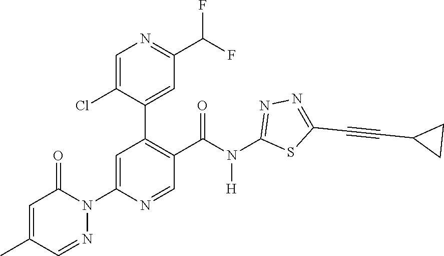 [SMILES: Cc1cnn(-c2cc(-c3cc(C(F)F)ncc3Cl)c(C(=O)Nc3nnc(C#CC4CC4)s3)cn2)c(=O)c1] |
| 471 | 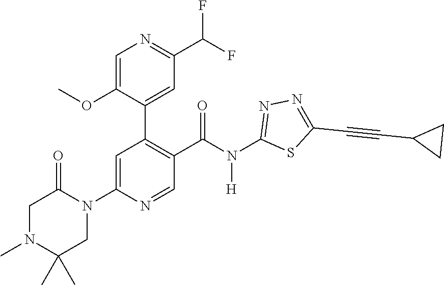 [SMILES: COc1cnc(C(F)F)cc1-c1cc(N2CC(C)(C)N(C)CC2=O)ncc1C(=O)Nc1nnc(C#CC2CC2)s1] |
| 472 | 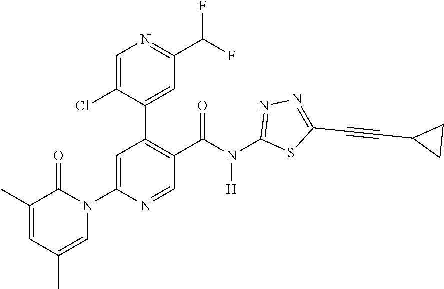 [SMILES: Cc1cc(C)c(=O)n(-c2cc(-c3cc(C(F)F)ncc3Cl)c(C(=O)Nc3nnc(C#CC4CC4)s3)cn2)c1] |
| 473 | 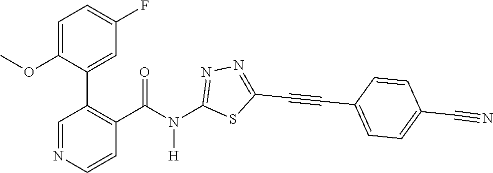 [SMILES: COc1ccc(F)cc1-c1cnccc1C(=O)Nc1nnc(C#Cc2ccc(C#N)cc2)s1] |
| 474 | 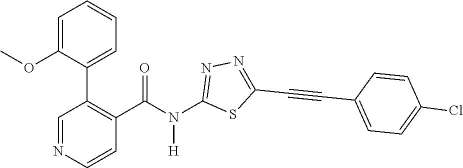 [SMILES: COc1ccccc1-c1cnccc1C(=O)Nc1nnc(C#Cc2ccc(Cl)cc2)s1] |
| 475 | 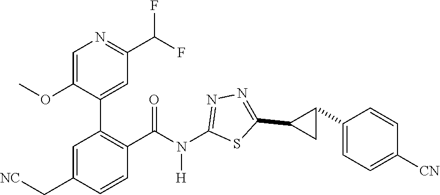 Racemic |
| 476 | 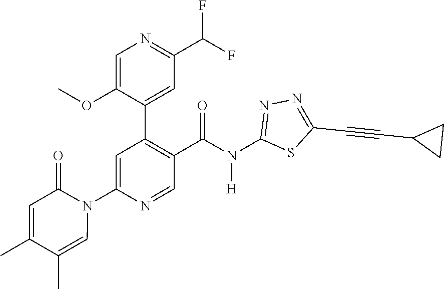 [SMILES: COc1cnc(C(F)F)cc1-c1cc(-n2cc(C)c(C)cc2=O)ncc1C(=O)Nc1nnc(C#CC2CC2)s1] |
| 477 | 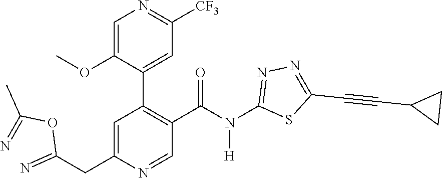 [SMILES: COc1cnc(C(F)(F)F)cc1-c1cc(CC(=N)OC(C)=N)ncc1C(=O)Nc1nnc(C#CC2CC2)s1] |
| 478 | 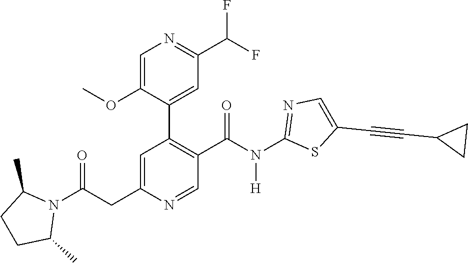 [SMILES: COc1cnc(C(F)F)cc1-c1cc(CC(=O)N2[C@H](C)CC[C@H]2C)ncc1C(=O)Nc1ncc(C#CC2CC2)s1] |
| 479 | 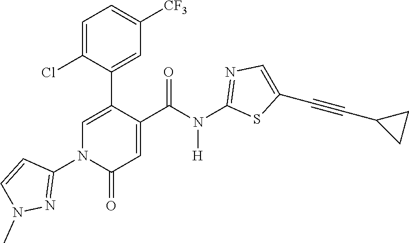 [SMILES: Cn1ccc(-n2cc(-c3cc(C(F)(F)F)ccc3Cl)c(C(=O)Nc3ncc(C#CC4CC4)s3)cc2=O)n1] |
| 480 | 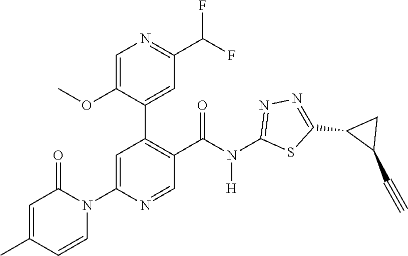 [SMILES: C#C[C@@H]1C[C@H]1c1nnc(NC(=O)c2cnc(-n3ccc(C)cc3=O)cc2-c2cc(C(F)F)ncc2OC)s1] |
| 481 | 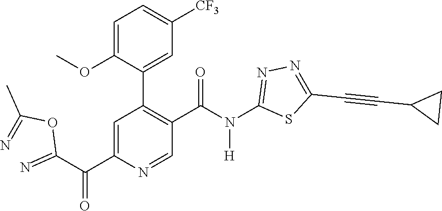 [SMILES: COc1ccc(C(F)(F)F)cc1-c1cc(C(=O)C(=N)OC(C)=N)ncc1C(=O)Nc1nnc(C#CC2CC2)s1] |
| 482 | 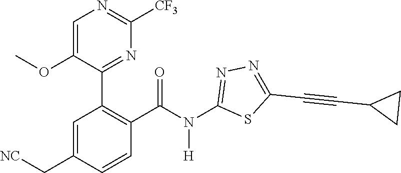 [SMILES: COc1cnc(C(F)(F)F)nc1-c1cc(CC#N)ccc1C(=O)Nc1nnc(C#CC2CC2)s1] |
| 483 | 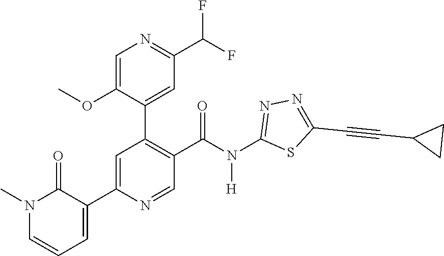 [SMILES: COc1cnc(C(F)F)cc1-c1cc(-c2cccn(C)c2=O)ncc1C(=O)Nc1nnc(C#CC2CC2)s1] |
| 484 | 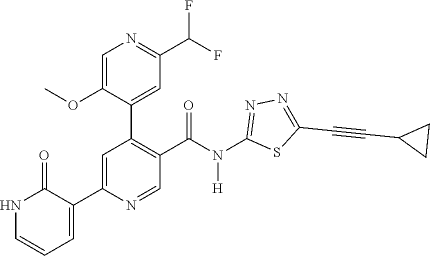 [SMILES: COc1cnc(C(F)F)cc1-c1cc(-c2ccc[nH]c2=O)ncc1C(=O)Nc1nnc(C#CC2CC2)s1] |
| 485 | 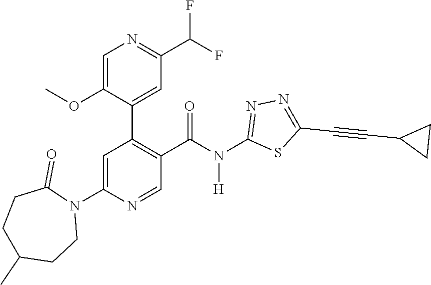 [SMILES: COc1cnc(C(F)F)cc1-c1cc(N2CCC(C)CCC2=O)ncc1C(=O)Nc1nnc(C#CC2CC2)s1] Racemic |
| 486 | 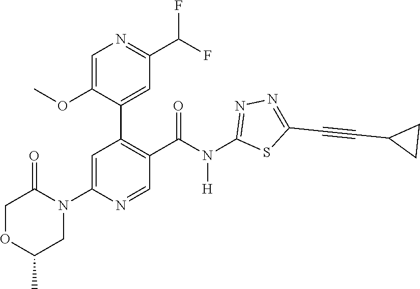 [SMILES: COc1cnc(C(F)F)cc1-c1cc(N2C[C@H](C)OCC2=O)ncc1C(=O)Nc1nnc(C#CC2CC2)s1] Chiral |
| 487 | 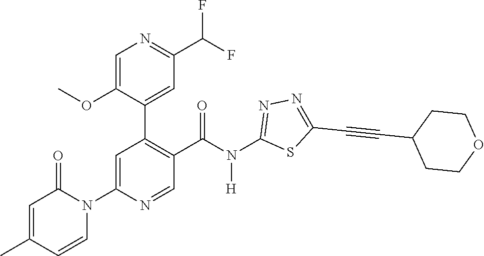 [SMILES: COc1cnc(C(F)F)cc1-c1cc(-n2ccc(C)cc2=O)ncc1C(=O)Nc1nnc(C#CC2CCOCC2)s1] |
| 488 | 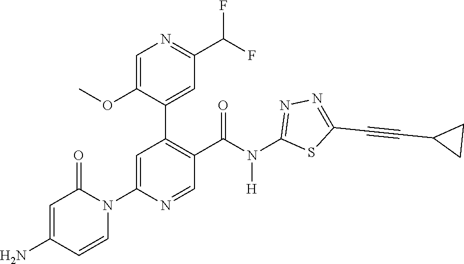 [SMILES: COc1cnc(C(F)F)cc1-c1cc(-n2ccc(N)cc2=O)ncc1C(=O)Nc1nnc(C#CC2CC2)s1] |
| 489 | 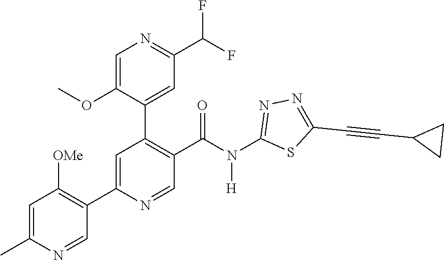 [SMILES: COc1cc(C)ncc1-c1cc(-c2cc(C(F)F)ncc2OC)c(C(=O)Nc2nnc(C#CC3CC3)s2)cn1] |
| 490 | 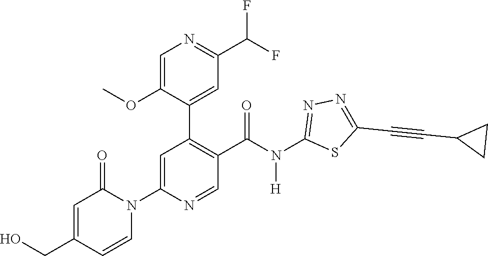 [SMILES: COc1cnc(C(F)F)cc1-c1cc(-n2ccc(CO)cc2=O)ncc1C(=O)Nc1nnc(C#CC2CC2)s1] |
| 491 | 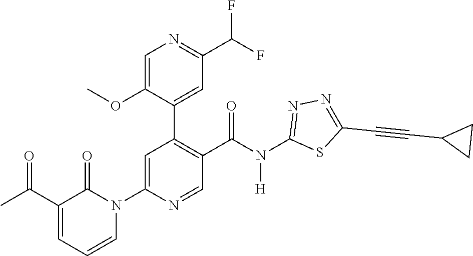 [SMILES: COc1cnc(C(F)F)cc1-c1cc(-n2cccc(C(C)=O)c2=O)ncc1C(=O)Nc1nnc(C#CC2CC2)s1] |
| 492 | 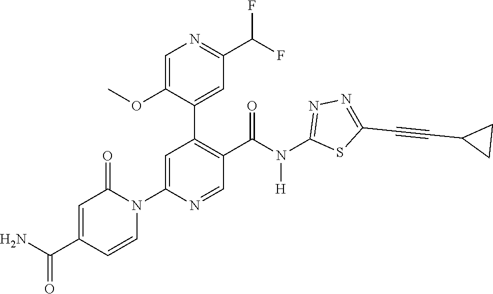 [SMILES: COc1cnc(C(F)F)cc1-c1cc(-n2ccc(C(N)=O)cc2=O)ncc1C(=O)Nc1nnc(C#CC2CC2)s1] |
| 493 | 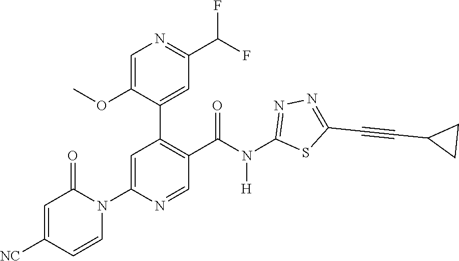 [SMILES: COc1cnc(C(F)F)cc1-c1cc(-n2ccc(C#N)cc2=O)ncc1C(=O)Nc1nnc(C#CC2CC2)s1] |
| 494 | 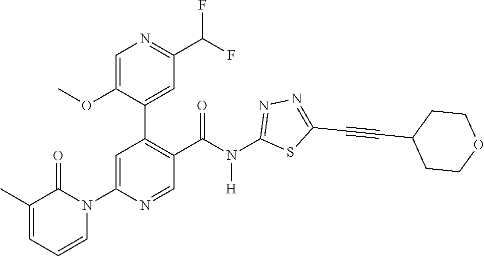 [SMILES: COc1cnc(C(F)F)cc1-c1cc(-n2cccc(C)c2=O)ncc1C(=O)Nc1nnc(C#CC2CCOCC2)s1] |
| 495 | 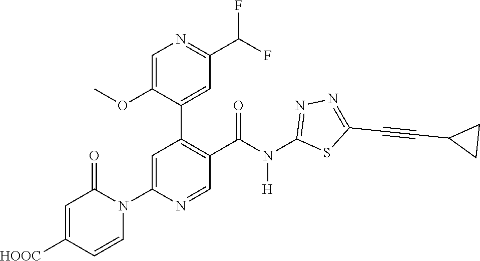 [SMILES: COc1cnc(C(F)F)cc1-c1cc(-n2ccc(C(=O)O)cc2=O)ncc1C(=O)Nc1nnc(C#CC2CC2)s1] |
| 496 | 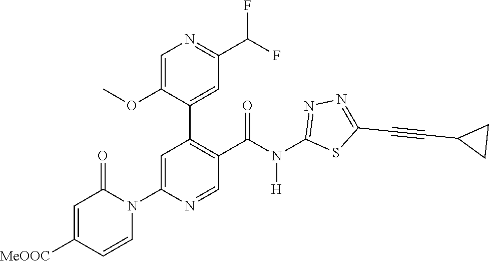 [SMILES: COC(=O)c1ccn(-c2cc(-c3cc(C(F)F)ncc3OC)c(C(=O)Nc3nnc(C#CC4CC4)s3)cn2)c(=O)c1] |
| 497 | 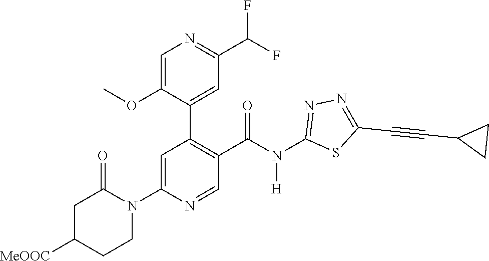 [SMILES: COC(=O)C1CCN(c2cc(-c3cc(C(F)F)ncc3OC)c(C(=O)Nc3nnc(C#CC4CC4)s3)cn2)C(=O)C1] |
| 498 | 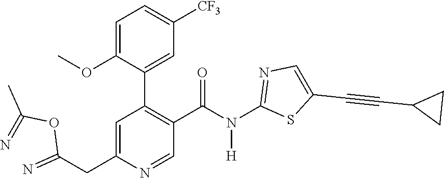 [SMILES: COc1ccc(C(F)(F)F)cc1-c1cc(CC(=N)OC(C)=N)ncc1C(=O)Nc1ncc(C#CC2CC2)s1] |
| 499 | 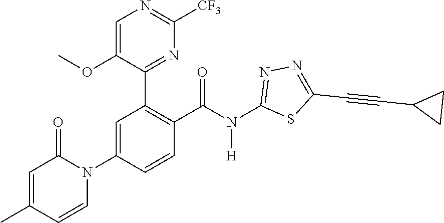 [SMILES: COc1cnc(C(F)(F)F)nc1-c1cc(-n2ccc(C)cc2=O)ccc1C(=O)Nc1nnc(C#CC2CC2)s1] |
| 500 | 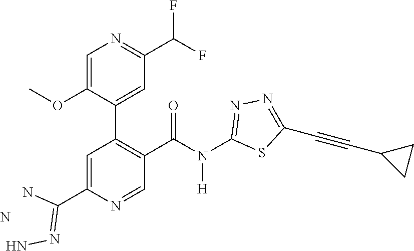 |
| 501 | 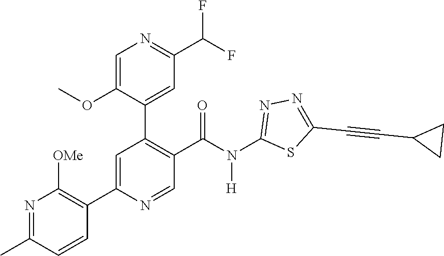 [SMILES: COc1cnc(C(F)F)cc1-c1cc(-c2ccc(C)nc2OC)ncc1C(=O)Nc1nnc(C#CC2CC2)s1] |
| 502 | 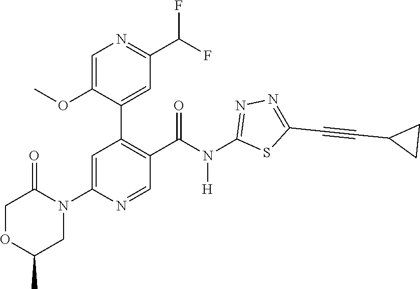 [SMILES: COc1cnc(C(F)F)cc1-c1cc(N2C[C@@H](C)OCC2=O)ncc1C(=O)Nc1nnc(C#CC2CC2)s1] Chiral |
| 503 | 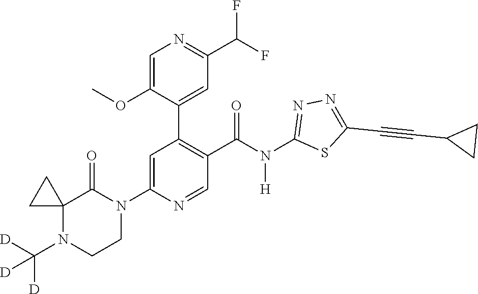 [SMILES: [2H]C([2H])([2H])N1CCN(c2cc(-c3cc(C(F)F)ncc3OC)c(C(=O)Nc3nnc(C#CC4CC4)s3)cn2)C(=O)C12CC2] |
| 504 | 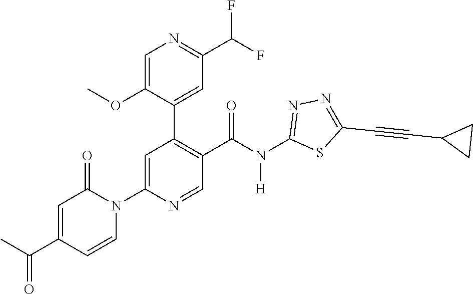 [SMILES: COc1cnc(C(F)F)cc1-c1cc(-n2ccc(C(C)=O)cc2=O)ncc1C(=O)Nc1nnc(C#CC2CC2)s1] |
| 505 | 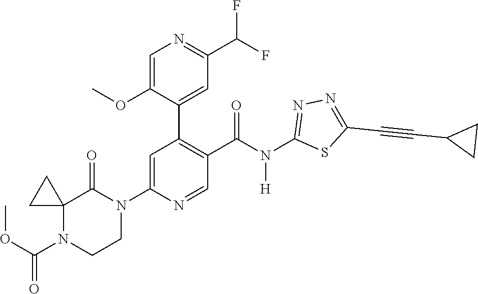 [SMILES: COC(=O)N1CCN(c2cc(-c3cc(C(F)F)ncc3OC)c(C(=O)Nc3nnc(C#CC4CC4)s3)cn2)C(=O)C12CC2] |
| 506 | 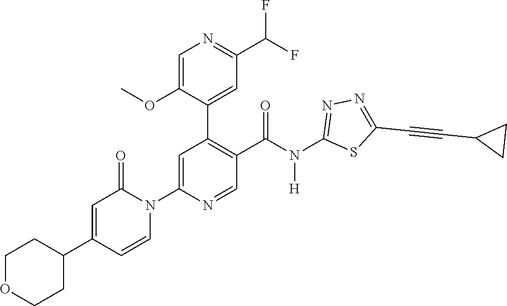 [SMILES: COc1cnc(C(F)F)cc1-c1cc(-n2ccc(C3CCOCC3)cc2=O)ncc1C(=O)Nc1nnc(C#CC2CC2)s1] |
| 507 | 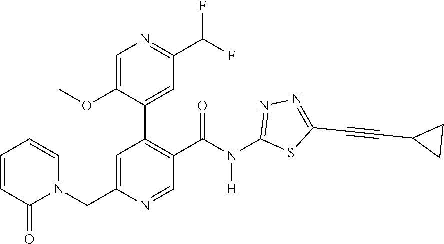 [SMILES: COc1cnc(C(F)F)cc1-c1cc(Cn2ccccc2=O)ncc1C(=O)Nc1nnc(C#CC2CC2)s1] |
| 508 | 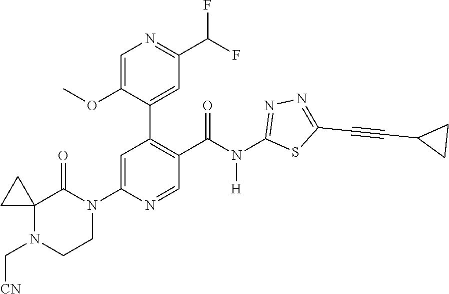 [SMILES: COc1cnc(C(F)F)cc1-c1cc(N2CCN(CC#N)C3(CC3)C2=O)ncc1C(=O)Nc1nnc(C#CC2CC2)s1] |
| 509 | 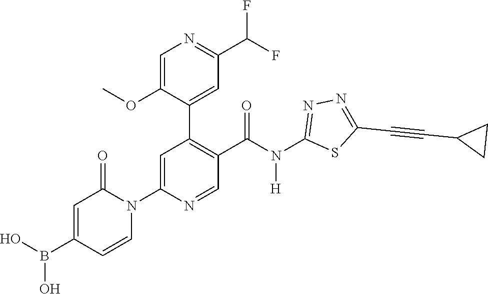 [SMILES: COc1cnc(C(F)F)cc1-c1cc(-n2ccc(B(O)O)cc2=O)ncc1C(=O)Nc1nnc(C#CC2CC2)s1] |
| 510 | 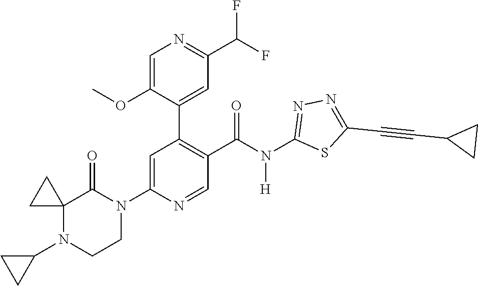 [SMILES: COc1cnc(C(F)F)cc1-c1cc(N2CCN(C3CC3)C3(CC3)C2=O)ncc1C(=O)Nc1nnc(C#CC2CC2)s1] |
| 511 | 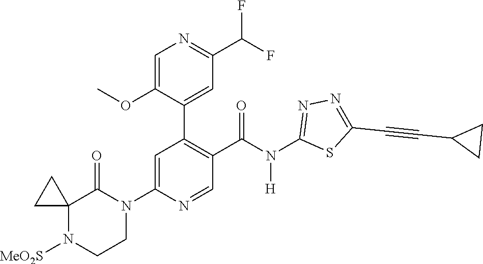 [SMILES: COc1cnc(C(F)F)cc1-c1cc(N2CCN(S(C)(=O)=O)C3(CC3)C2=O)ncc1C(=O)Nc1nnc(C#CC2CC2)s1] |
| 512 | 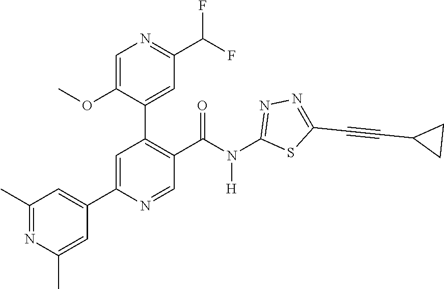 [SMILES: COc1cnc(C(F)F)cc1-c1cc(-c2cc(C)nc(C)c2)ncc1C(=O)Nc1nnc(C#CC2CC2)s1] |
| 513 | 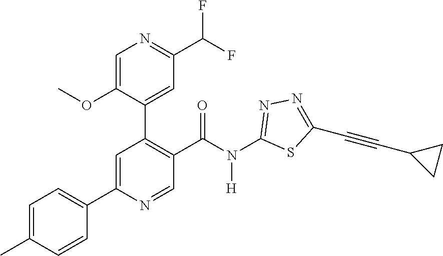 [SMILES: COc1cnc(C(F)F)cc1-c1cc(-c2ccc(C)cc2)ncc1C(=O)Nc1nnc(C#CC2CC2)s1] |
| 514 | 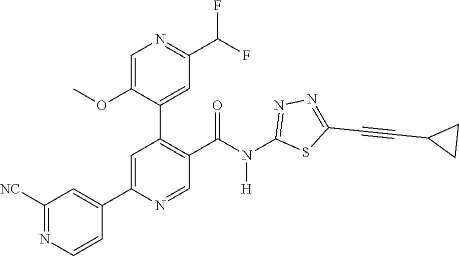 [SMILES: COc1cnc(C(F)F)cc1-c1cc(-c2ccnc(C#N)c2)ncc1C(=O)Nc1nnc(C#CC2CC2)s1] |
| 515 | 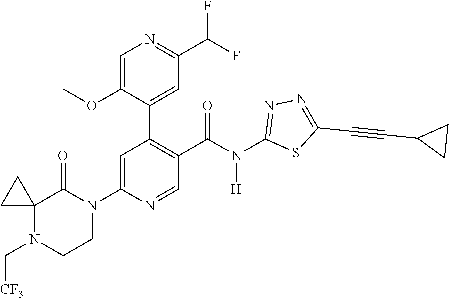 [SMILES: COc1cnc(C(F)F)cc1-c1cc(N2CCN(CC(F)(F)F)C3(CC3)C2=O)ncc1C(=O)Nc1nnc(C#CC2CC2)s1] |
| 516 | 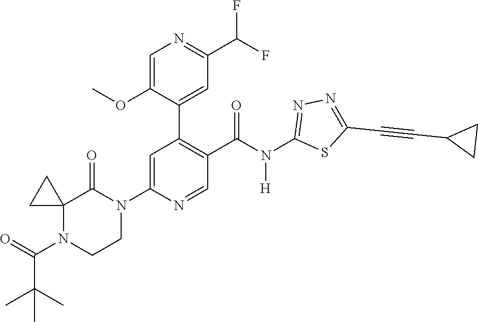 [SMILES: COc1cnc(C(F)F)cc1-c1cc(N2CCN(C(=O)C(C)(C)C)C3(CC3)C2=O)ncc1C(=O)Nc1nnc(C#CC2CC2)s1] |
| 517 | 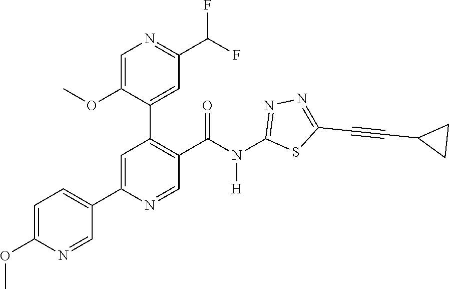 [SMILES: COc1ccc(-c2cc(-c3cc(C(F)F)ncc3OC)c(C(=O)Nc3nnc(C#CC4CC4)s3)cn2)cn1] |
| 518 | 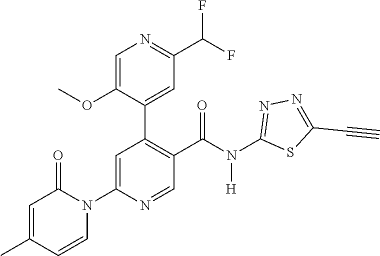 [SMILES: C#Cc1nnc(NC(=O)c2cnc(-n3ccc(C)cc3=O)cc2-c2cc(C(F)F)ncc2OC)s1] |
| 519 | 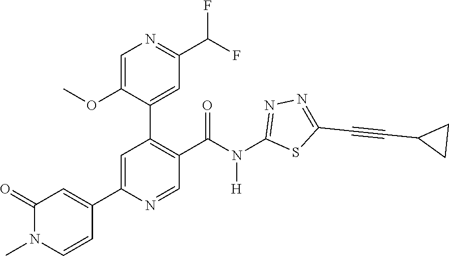 [SMILES: COc1cnc(C(F)F)cc1-c1cc(-c2ccn(C)c(=O)c2)ncc1C(=O)Nc1nnc(C#CC2CC2)s1] |
| 520 | 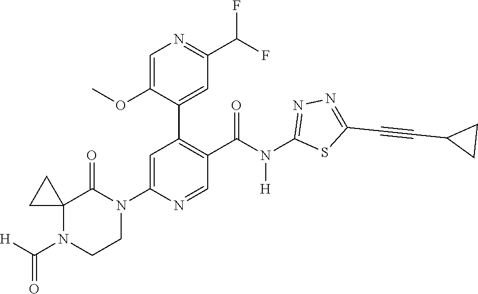 [SMILES: COc1cnc(C(F)F)cc1-c1cc(N2CCN(C=O)C3(CC3)C2=O)ncc1C(=O)Nc1nnc(C#CC2CC2)s1] |
| 521 | 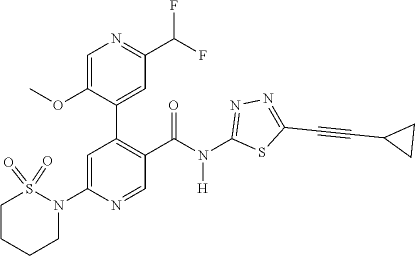 [SMILES: COc1cnc(C(F)F)cc1-c1cc(N2CCCCS2(=O)=O)ncc1C(=O)Nc1nnc(C#CC2CC2)s1] |
| 522 | 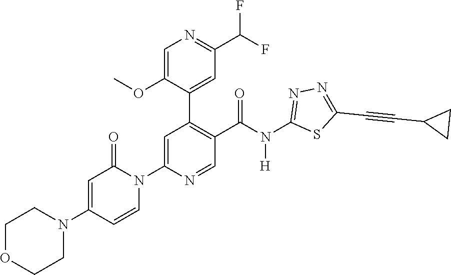 [SMILES: COc1cnc(C(F)F)cc1-c1cc(-n2ccc(N3CCOCC3)cc2=O)ncc1C(=O)Nc1nnc(C#CC2CC2)s1] |
| 523 | 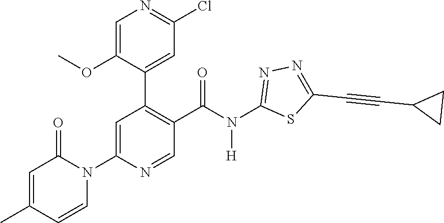 [SMILES: COc1cnc(Cl)cc1-c1cc(-n2ccc(C)cc2=O)ncc1C(=O)Nc1nnc(C#CC2CC2)s1] |
| 524 | 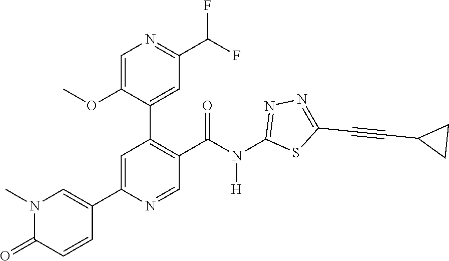 [SMILES: COc1cnc(C(F)F)cc1-c1cc(-c2ccc(=O)n(C)c2)ncc1C(=O)Nc1nnc(C#CC2CC2)s1] |
| 525 | 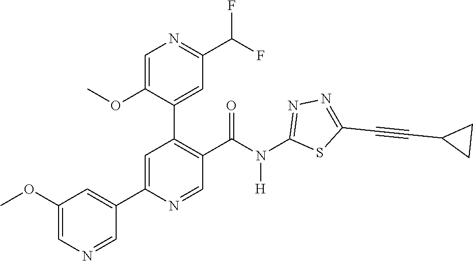 [SMILES: COc1cncc(-c2cc(-c3cc(C(F)F)ncc3OC)c(C(=O)Nc3nnc(C#CC4CC4)s3)cn2)c1] |
| 526 | 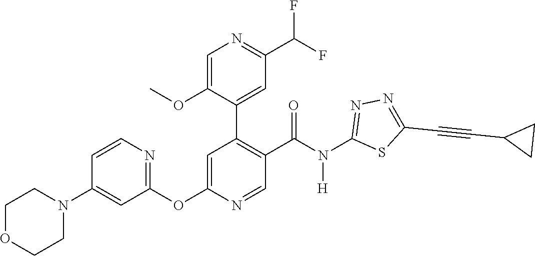 [SMILES: COc1cnc(C(F)F)cc1-c1cc(Oc2cc(N3CCOCC3)ccn2)ncc1C(=O)Nc1nnc(C#CC2CC2)s1] |
| 527 | 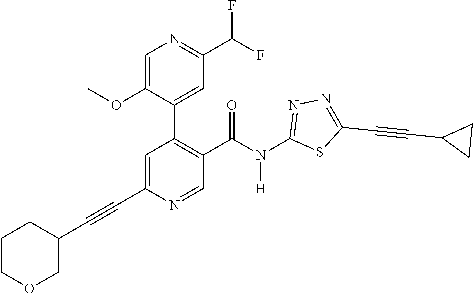 [SMILES: COc1cnc(C(F)F)cc1-c1cc(C#CC2CCCOC2)ncc1C(=O)Nc1nnc(C#CC2CC2)s1] Racemic |
| 528 | 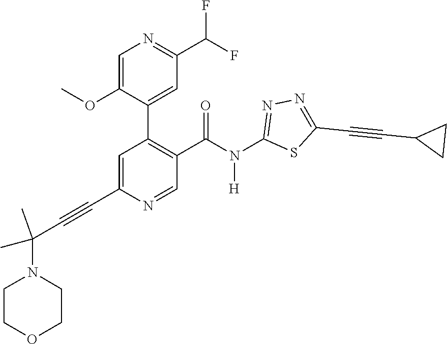 [SMILES: COc1cnc(C(F)F)cc1-c1cc(C#CC(C)(C)N2CCOCC2)ncc1C(=O)Nc1nnc(C#CC2CC2)s1] |
| 529 | 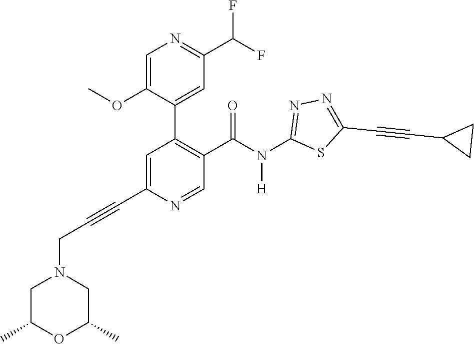 [SMILES: COc1cnc(C(F)F)cc1-c1cc(C#CCN2C[C@@H](C)O[C@@H](C)C2)ncc1C(=O)Nc1nnc(C#CC2CC2)s1] Chiral |
| 530 | 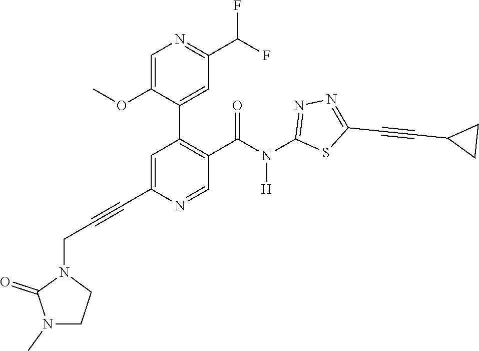 [SMILES: COc1cnc(C(F)F)cc1-c1cc(C#CCN2CCN(C)C2=O)ncc1C(=O)Nc1nnc(C#CC2CC2)s1] |
| 531 | 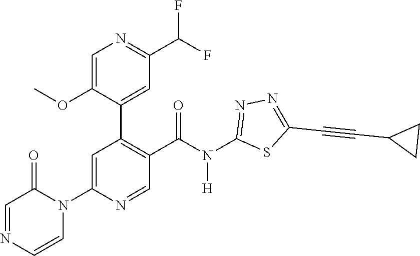 [SMILES: COc1cnc(C(F)F)cc1-c1cc(-n2ccncc2=O)ncc1C(=O)Nc1nnc(C#CC2CC2)s1] |
| 532 | 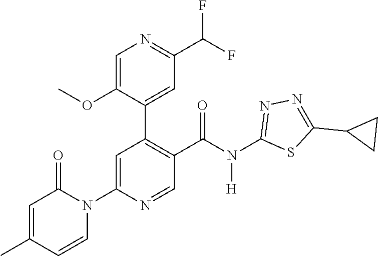 [SMILES: COc1cnc(C(F)F)cc1-c1cc(-n2ccc(C)cc2=O)ncc1C(=O)Nc1nnc(C2CC2)s1] |
| 533 | 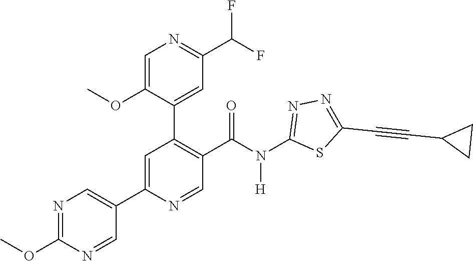 [SMILES: COc1ncc(-c2cc(-c3cc(C(F)F)ncc3OC)c(C(=O)Nc3nnc(C#CC4CC4)s3)cn2)cn1] |
| 534 | 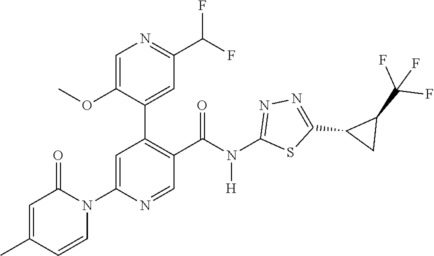 [SMILES: COc1cnc(C(F)F)cc1-c1cc(-n2ccc(C)cc2=O)ncc1C(=O)Nc1nnc([C@H]2C[C@@H]2C(F)(F)F)s1] Chiral |
| 535 | 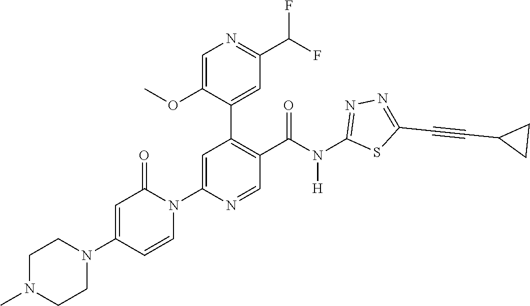 [SMILES: COc1cnc(C(F)F)cc1-c1cc(-n2ccc(N3CCN(C)CC3)cc2=O)ncc1C(=O)Nc1nnc(C#CC2CC2)s1] |
| 536 | 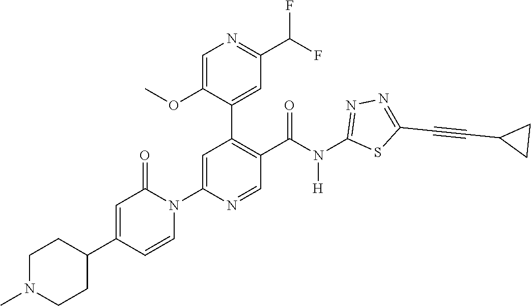 [SMILES: COc1cnc(C(F)F)cc1-c1cc(-n2ccc(C3CCN(C)CC3)cc2=O)ncc1C(=O)Nc1nnc(C#CC2CC2)s1] |
| 537 | 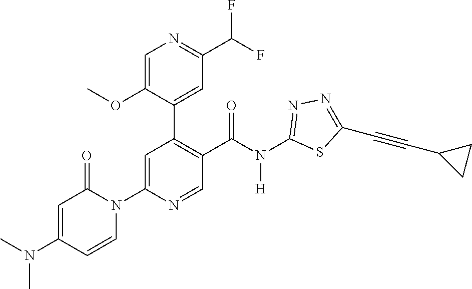 [SMILES: COc1cnc(C(F)F)cc1-c1cc(-n2ccc(N(C)C)cc2=O)ncc1C(=O)Nc1nnc(C#CC2CC2)s1] |
| 538 | 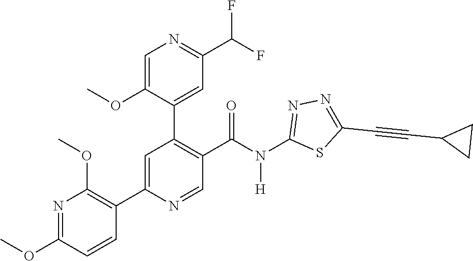 [SMILES: COc1ccc(-c2cc(-c3cc(C(F)F)ncc3OC)c(C(=O)Nc3nnc(C#CC4CC4)s3)cn2)c(OC)n1] |
| 539 | 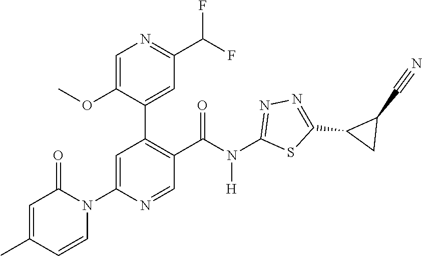 [SMILES: COc1cnc(C(F)F)cc1-c1cc(-n2ccc(C)cc2=O)ncc1C(=O)Nc1nnc([C@H]2C[C@@H]2C#N)s1] Racemic |
| 540 | 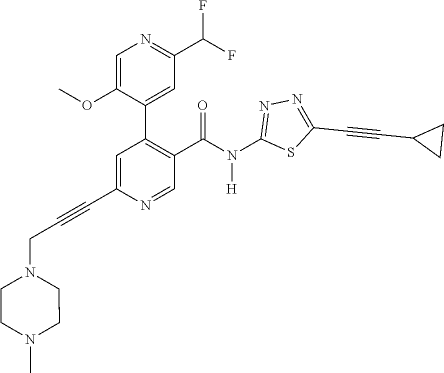 [SMILES: COc1cnc(C(F)F)cc1-c1cc(C#CCN2CCN(C)CC2)ncc1C(=O)Nc1nnc(C#CC2CC2)s1] |
| 541 | 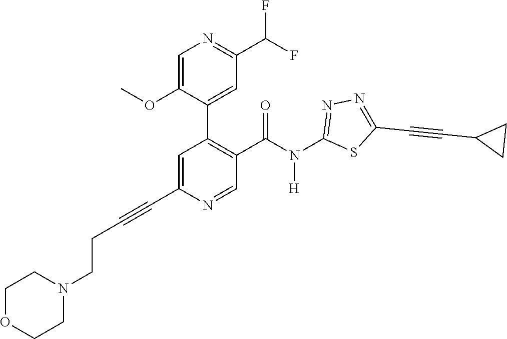 [SMILES: COc1cnc(C(F)F)cc1-c1cc(C#CCCN2CCOCC2)ncc1C(=O)Nc1nnc(C#CC2CC2)s1] |
| 542 | 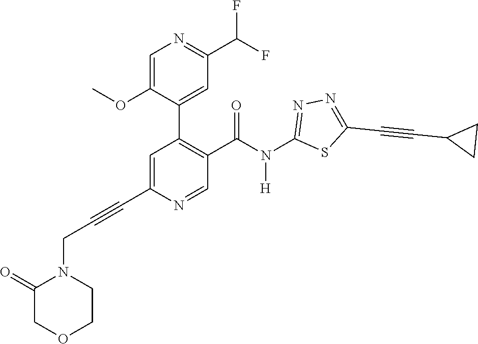 [SMILES: COc1cnc(C(F)F)cc1-c1cc(C#CCN2CCOCC2=O)ncc1C(=O)Nc1nnc(C#CC2CC2)s1] |
| 543 | 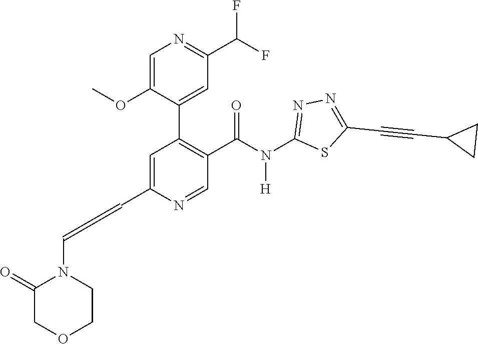 [SMILES: COc1cnc(C(F)F)cc1-c1cc(C=C=CN2CCOCC2=O)ncc1C(=O)Nc1nnc(C#CC2CC2)s1] |
| 544 | 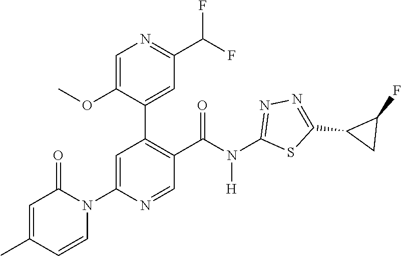 [SMILES: COc1cnc(C(F)F)cc1-c1cc(-n2ccc(C)cc2=O)ncc1C(=O)Nc1nnc([C@H]2C[C@@H]2F)s1] Chiral |
| 545 | 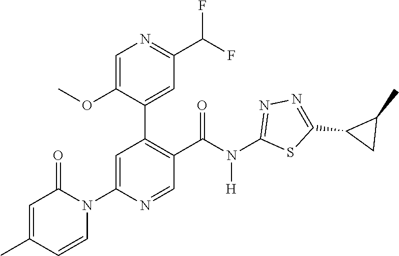 Chiral |
| 546 | 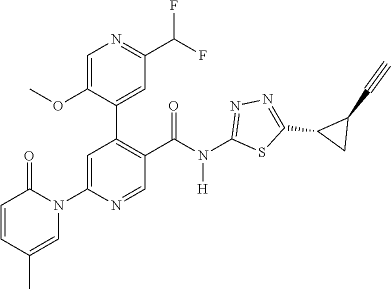 [SMILES: C#C[C@H]1C[C@@H]1c1nnc(NC(=O)c2cnc(-n3cc(C)ccc3=O)cc2-c2cc(C(F)F)ncc2OC)s1] Chiral |
| 547 | 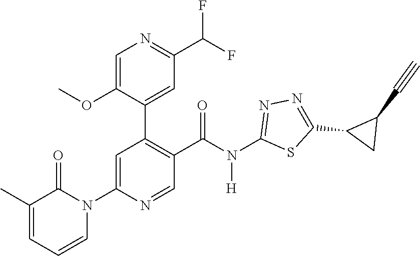 [SMILES: C#C[C@H]1C[C@@H]1c1nnc(NC(=O)c2cnc(-n3cccc(C)c3=O)cc2-c2cc(C(F)F)ncc2OC)s1] Chiral |
| 548 | 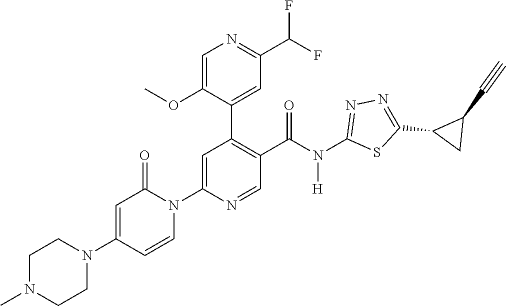 [SMILES: C#C[C@H]1C[C@@H]1c1nnc(NC(=O)c2cnc(-n3ccc(N4CCN(C)CC4)cc3=O)cc2-c2cc(C(F)F)ncc2OC)s1] Chiral |
| 549 | 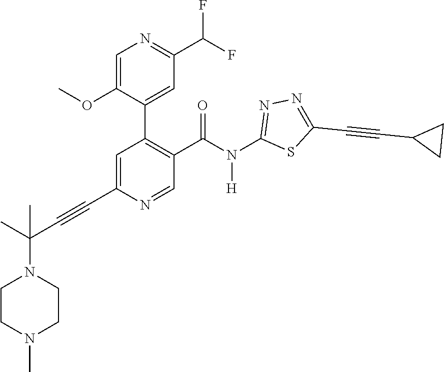 [SMILES: COc1cnc(C(F)F)cc1-c1cc(C#CC(C)(C)N2CCN(C)CC2)ncc1C(=O)Nc1nnc(C#CC2CC2)s1] |
| 550 | 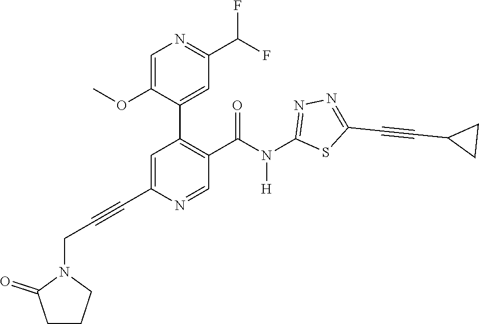 [SMILES: COc1cnc(C(F)F)cc1-c1cc(C#CCN2CCCC2=O)ncc1C(=O)Nc1nnc(C#CC2CC2)s1] |
| 551 | 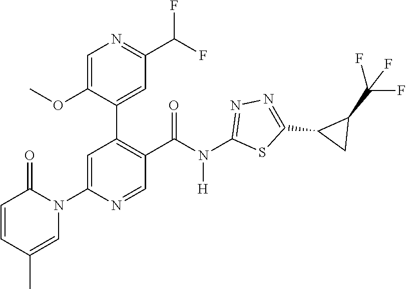 [SMILES: COc1cnc(C(F)F)cc1-c1cc(-n2cc(C)ccc2=O)ncc1C(=O)Nc1nnc([C@H]2C[C@@H]2C(F)(F)F)s1] Chiral |
| 552 | 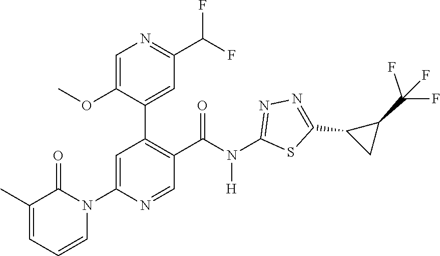 [SMILES: COc1cnc(C(F)F)cc1-c1cc(-n2cccc(C)c2=O)ncc1C(=O)Nc1nnc([C@H]2C[C@@H]2C(F)(F)F)s1] Chiral |
| 553 | 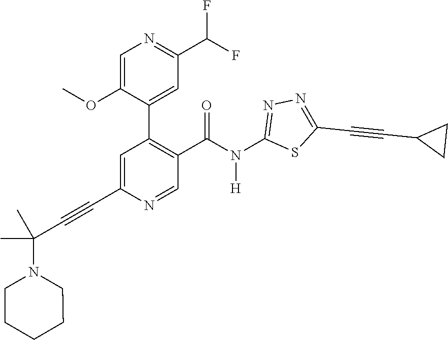 [SMILES: COc1cnc(C(F)F)cc1-c1cc(C#CC(C)(C)N2CCCCC2)ncc1C(=O)Nc1nnc(C#CC2CC2)s1] |
| 554 | 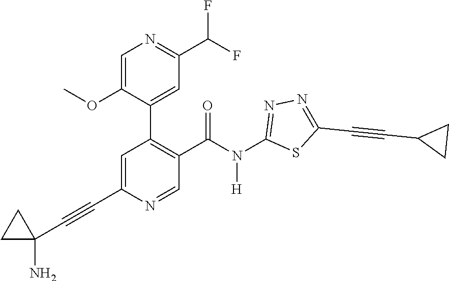 [SMILES: COc1cnc(C(F)F)cc1-c1cc(C#CC2(N)CC2)ncc1C(=O)Nc1nnc(C#CC2CC2)s1] |
| 555 | 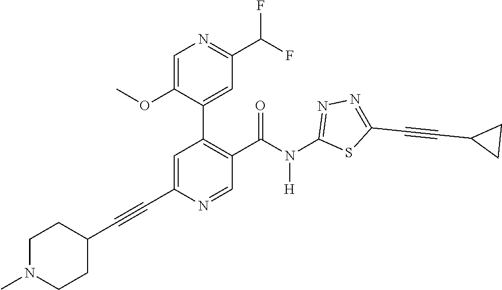 [SMILES: COc1cnc(C(F)F)cc1-c1cc(C#CC2CCN(C)CC2)ncc1C(=O)Nc1nnc(C#CC2CC2)s1] |
| 556 | 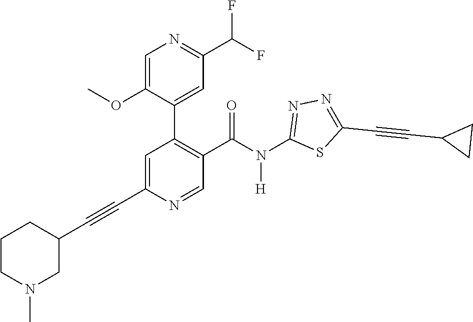 [SMILES: COc1cnc(C(F)F)cc1-c1cc(C#CC2CCCN(C)C2)ncc1C(=O)Nc1nnc(C#CC2CC2)s1] Racemic |
| 557 | 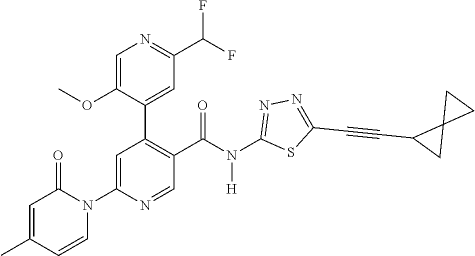 [SMILES: COc1cnc(C(F)F)cc1-c1cc(-n2ccc(C)cc2=O)ncc1C(=O)Nc1nnc(C#CC2CC23CC3)s1] Racemic |
| 558 | 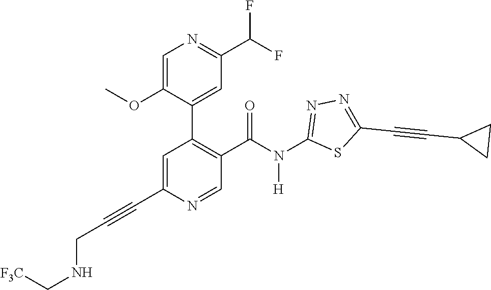 [SMILES: COc1cnc(C(F)F)cc1-c1cc(C#CCNCC(F)(F)F)ncc1C(=O)Nc1nnc(C#CC2CC2)s1] |
| 559 | 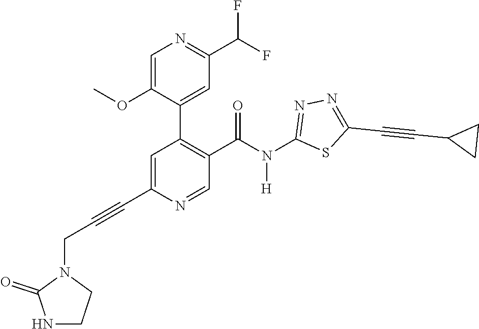 [SMILES: COc1cnc(C(F)F)cc1-c1cc(C#CCN2CCNC2=O)ncc1C(=O)Nc1nnc(C#CC2CC2)s1] |
| 560 | 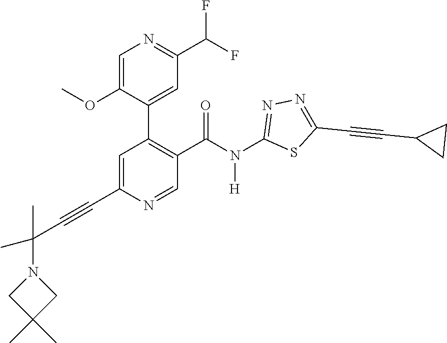 [SMILES: COc1cnc(C(F)F)cc1-c1cc(C#CC(C)(C)N2CC(C)(C)C2)ncc1C(=O)Nc1nnc(C#CC2CC2)s1] |
| 561 | 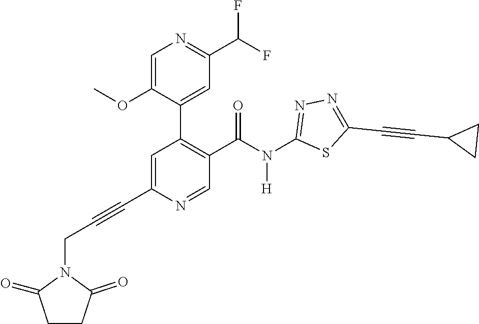 [SMILES: COc1cnc(C(F)F)cc1-c1cc(C#CCN2C(=O)CCC2=O)ncc1C(=O)Nc1nnc(C#CC2CC2)s1] |
| 562 | 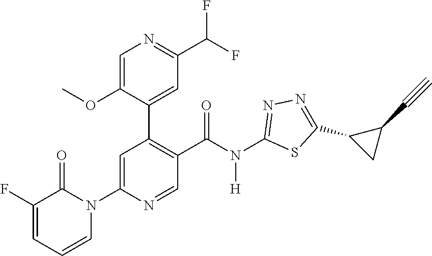 [SMILES: C#C[C@H]1C[C@@H]1c1nnc(NC(=O)c2cnc(-n3cccc(F)c3=O)cc2-c2cc(C(F)F)ncc2OC)s1] Chiral |
| 563 | 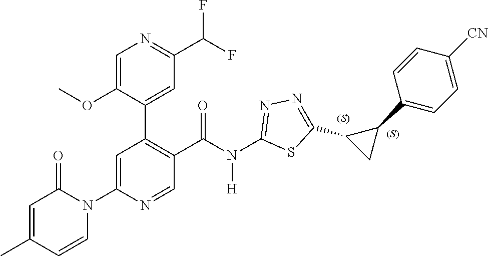 |
| 564 | 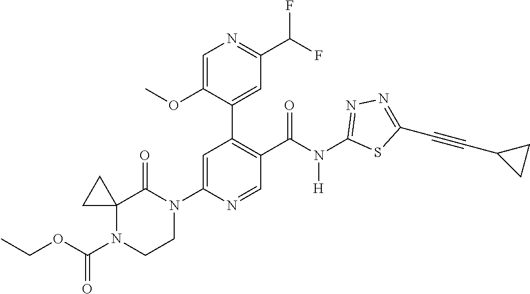 [SMILES: CCOC(=O)N1CCN(c2cc(-c3cc(C(F)F)ncc3OC)c(C(=O)Nc3nnc(C#CC4CC4)s3)cn2)C(=O)C12CC2] |
| 565 | 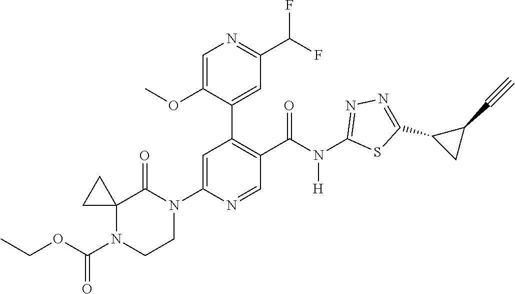 [SMILES: C#C[C@H]1C[C@@H]1c1nnc(NC(=O)c2cnc(N3CCN(C(=O)OCC)C4(CC4)C3=O)cc2-c2cc(C(F)F)ncc2OC)s1] |
| 566 | 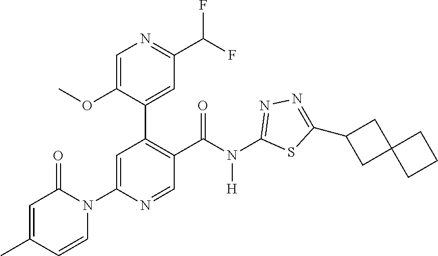 [SMILES: COc1cnc(C(F)F)cc1-c1cc(-n2ccc(C)cc2=O)ncc1C(=O)Nc1nnc(C2CC3(CCC3)C2)s1] |
| 567 | 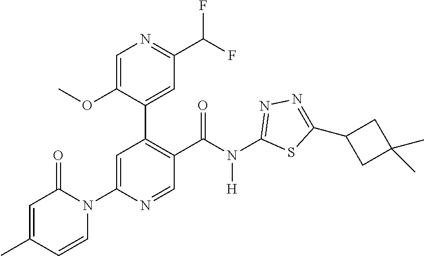 |
| 568 | 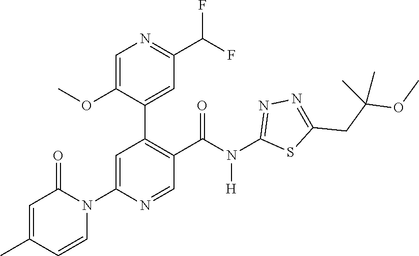 |
| 569 | 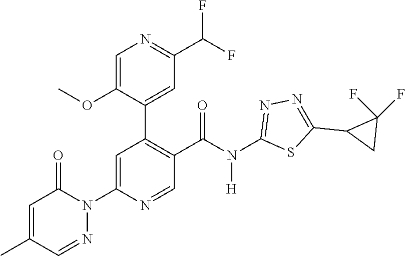 [SMILES: COc1cnc(C(F)F)cc1-c1cc(-n2ncc(C)cc2=O)ncc1C(=O)Nc1nnc(C2CC2(F)F)s1] Racemic |
| 570 | 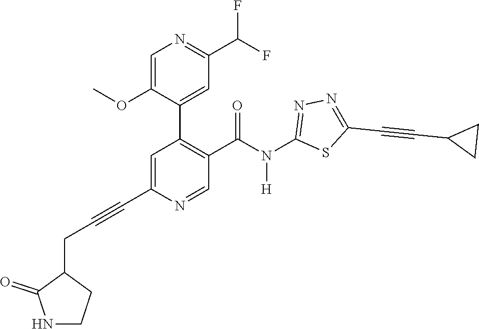 [SMILES: COc1cnc(C(F)F)cc1-c1cc(C#CCC2CCNC2=O)ncc1C(=O)Nc1nnc(C#CC2CC2)s1] Racemic |
| 571 | 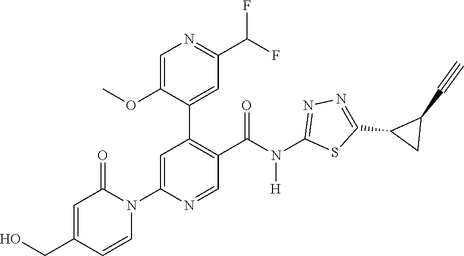 [SMILES: C#C[C@H]1C[C@@H]1c1nnc(NC(=O)c2cnc(-n3ccc(CO)cc3=O)cc2-c2cc(C(F)F)ncc2OC)s1] Chiral |
| 572 | 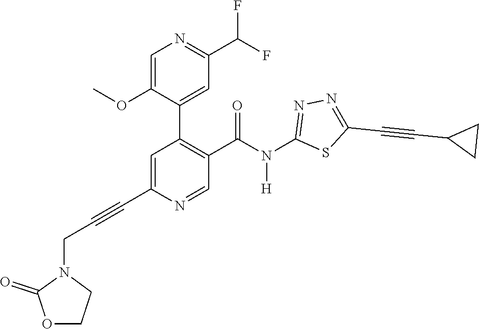 [SMILES: COc1cnc(C(F)F)cc1-c1cc(C#CCN2CCOC2=O)ncc1C(=O)Nc1nnc(C#CC2CC2)s1] |
| 573 | 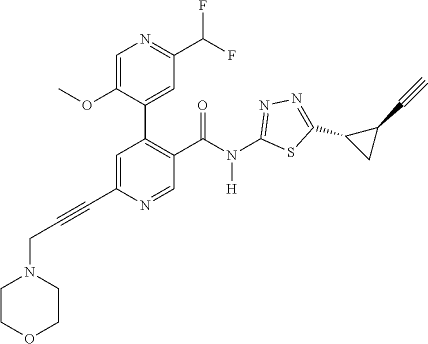 [SMILES: C#C[C@H]1C[C@@H]1c1nnc(NC(=O)c2cnc(C#CCN3CCOCC3)cc2-c2cc(C(F)F)ncc2OC)s1] Chiral |
| 574 | 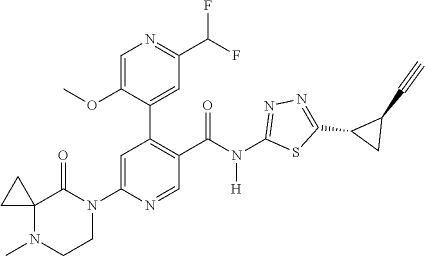 [SMILES: C#C[C@H]1C[C@@H]1c1nnc(NC(=O)c2cnc(N3CCN(C)C4(CC4)C3=O)cc2-c2cc(C(F)F)ncc2OC)s1] Chiral |
| 575 | 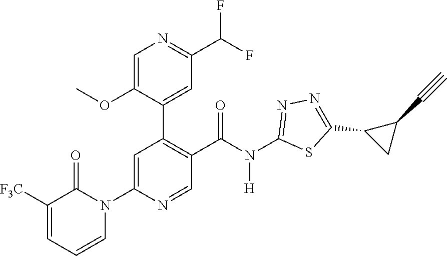 [SMILES: C#C[C@H]1C[C@@H]1c1nnc(NC(=O)c2cnc(-n3cccc(C(F)(F)F)c3=O)cc2-c2cc(C(F)F)ncc2OC)s1] Chiral |
| 576 | 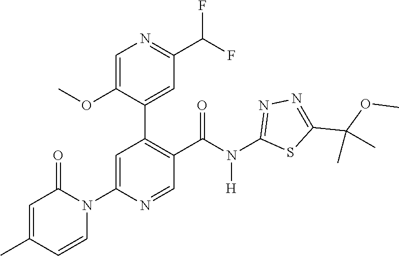 |
| 577 | 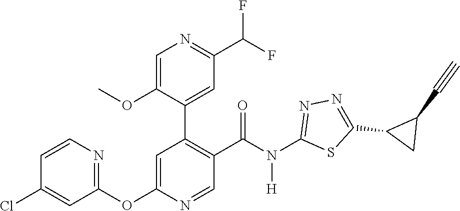 [SMILES: C#C[C@H]1C[C@@H]1c1nnc(NC(=O)c2cnc(Oc3cc(Cl)ccn3)cc2-c2cc(C(F)F)ncc2OC)s1] Chiral |
| 578 | 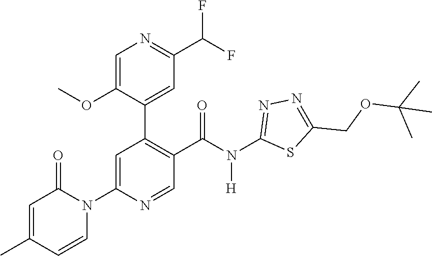 [SMILES: COc1cnc(C(F)F)cc1-c1cc(-n2ccc(C)cc2=O)ncc1C(=O)Nc1nnc(COC(C)(C)C)s1] |
| 579 | 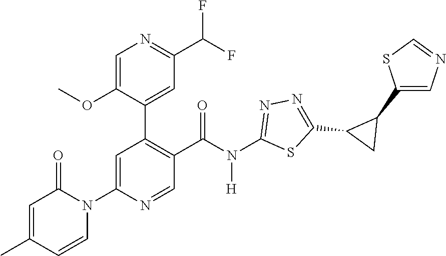 [SMILES: COc1cnc(C(F)F)cc1-c1cc(-n2ccc(C)cc2=O)ncc1C(=O)Nc1nnc([C@H]2C[C@@H]2c2cncs2)s1] Racemic |
| 580 | 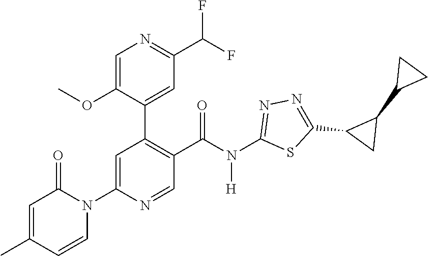 [SMILES: COc1cnc(C(F)F)cc1-c1cc(-n2ccc(C)cc2=O)ncc1C(=O)Nc1nnc([C@H]2C[C@@H]2C2CC2)s1] Racemic |
| 581 | 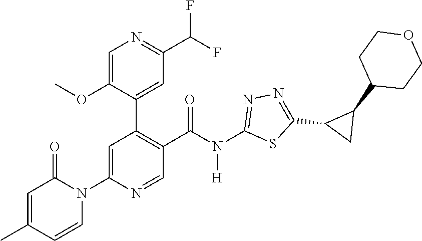 [SMILES: COc1cnc(C(F)F)cc1-c1cc(-n2ccc(C)cc2=O)ncc1C(=O)Nc1nnc([C@H]2C[C@@H]2C2CCOCC2)s1] Racemic |
| 582 | 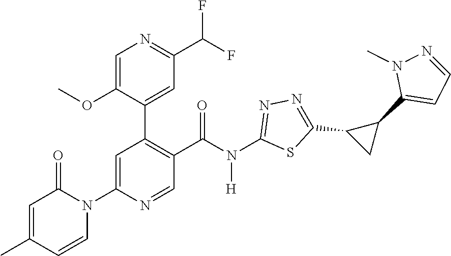 [SMILES: COc1cnc(C(F)F)cc1-c1cc(-n2ccc(C)cc2=O)ncc1C(=O)Nc1nnc([C@H]2C[C@@H]2c2ccnn2C)s1] Racemic |
| 583 | 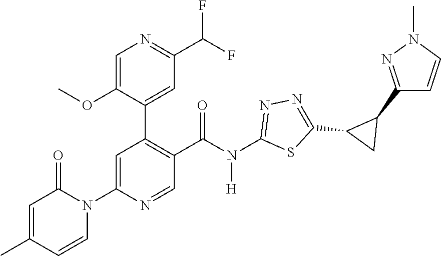 [SMILES: COc1cnc(C(F)F)cc1-c1cc(-n2ccc(C)cc2=O)ncc1C(=O)Nc1nnc([C@H]2C[C@@H]2c2ccn(C)n2)s1] Racemic |
| 584 | 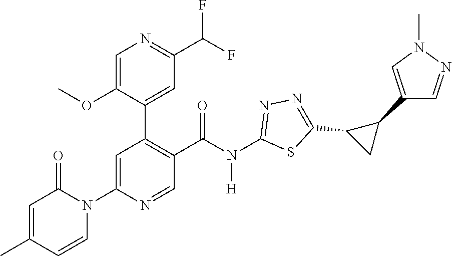 [SMILES: COc1cnc(C(F)F)cc1-c1cc(-n2ccc(C)cc2=O)ncc1C(=O)Nc1nnc([C@H]2C[C@@H]2c2cnn(C)c2)s1] Racemic |
| 585 | 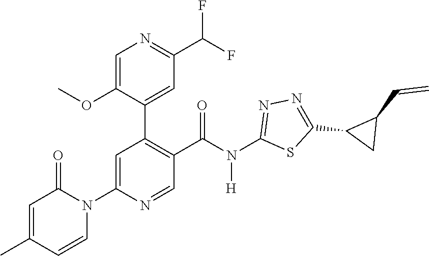 [SMILES: C=C[C@H]1C[C@@H]1c1nnc(NC(=O)c2cnc(-n3ccc(C)cc3=O)cc2-c2cc(C(F)F)ncc2OC)s1] Racemic |
| 586 | 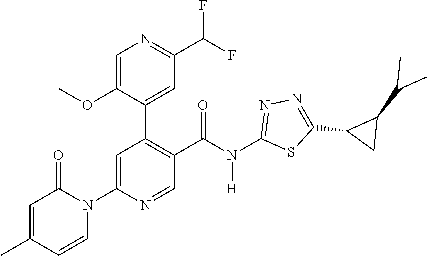 [SMILES: COc1cnc(C(F)F)cc1-c1cc(-n2ccc(C)cc2=O)ncc1C(=O)Nc1nnc([C@H]2C[C@@H]2C(C)C)s1] Racemic |
| 587 | 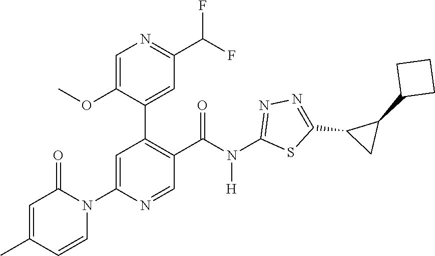 [SMILES: COc1cnc(C(F)F)cc1-c1cc(-n2ccc(C)cc2=O)ncc1C(=O)Nc1nnc([C@H]2C[C@@H]2C2CCC2)s1] Racemic |
| 588 | 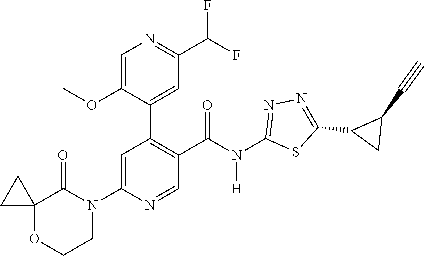 Chiral |
| 589 | 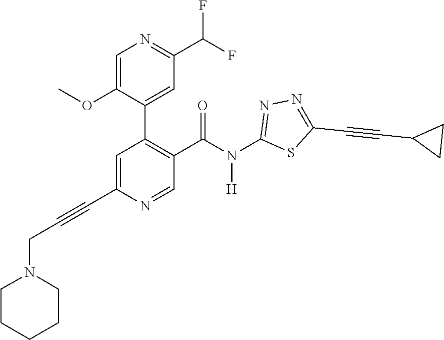 [SMILES: COc1cnc(C(F)F)cc1-c1cc(C#CCN2CCCCC2)ncc1C(=O)Nc1nnc(C#CC2CC2)s1] |
| 590 | 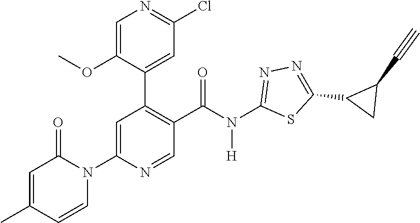 Chiral |
| 591 | 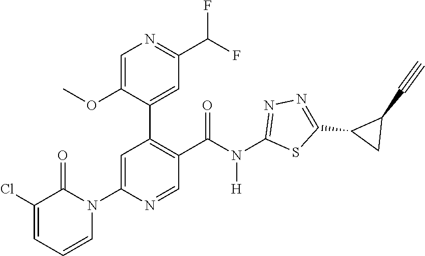 [SMILES: C#C[C@H]1C[C@@H]1c1nnc(NC(=O)c2cnc(-n3cccc(Cl)c3=O)cc2-c2cc(C(F)F)ncc2OC)s1] Chiral |
| 592 | 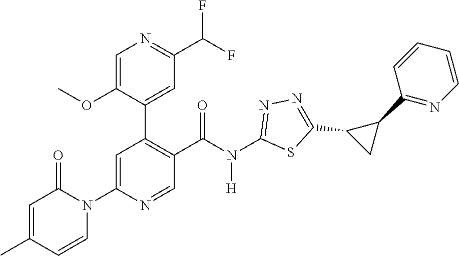 [SMILES: COc1cnc(C(F)F)cc1-c1cc(-n2ccc(C)cc2=O)ncc1C(=O)Nc1nnc([C@H]2C[C@@H]2c2ccccn2)s1] Racemic |
| 593 | 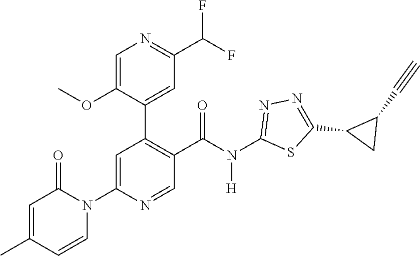 [SMILES: C#C[C@@H]1C[C@@H]1c1nnc(NC(=O)c2cnc(-n3ccc(C)cc3=O)cc2-c2cc(C(F)F)ncc2OC)s1] Racemic |
| 594 | 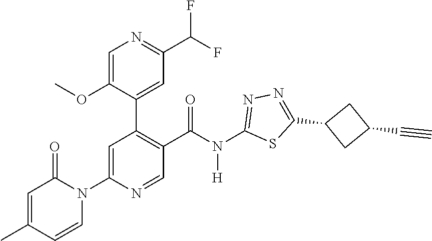 [SMILES: C#C[C@H]1C[C@@H](c2nnc(NC(=O)c3cnc(-n4ccc(C)cc4=O)cc3-c3cc(C(F)F)ncc3OC)s2)C1] Racemic |
| 595 | 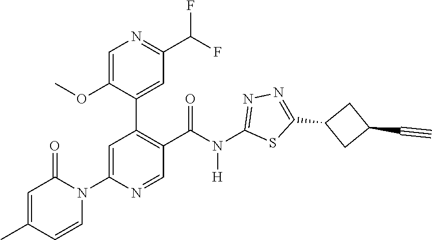 [SMILES: C#C[C@H]1C[C@H](c2nnc(NC(=O)c3cnc(-n4ccc(C)cc4=O)cc3-c3cc(C(F)F)ncc3OC)s2)C1] Racemic |
| 596 | 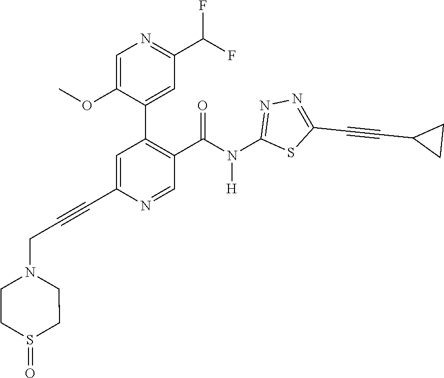 [SMILES: COc1cnc(C(F)F)cc1-c1cc(C#CCN2CCS(=O)CC2)ncc1C(=O)Nc1nnc(C#CC2CC2)s1] |
| 597 | 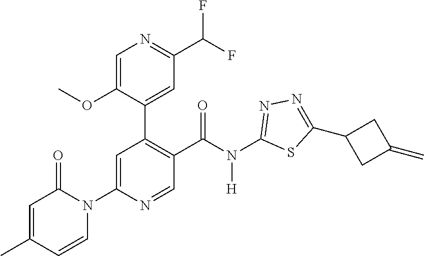 |
| 598 | 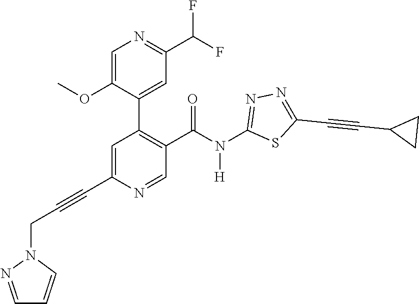 [SMILES: COc1cnc(C(F)F)cc1-c1cc(C#CCn2cccn2)ncc1C(=O)Nc1nnc(C#CC2CC2)s1] |
| 599 | 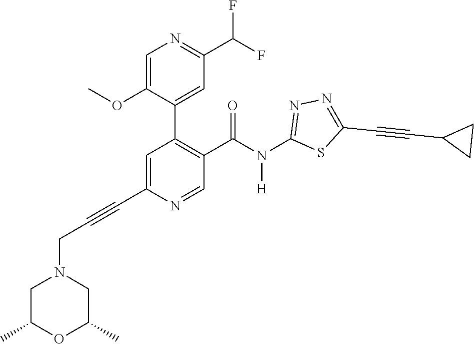 [SMILES: COc1cnc(C(F)F)cc1-c1cc(C#CCN2C[C@@H](C)O[C@@H](C)C2)ncc1C(=O)Nc1nnc(C#CC2CC2)s1] |
| 600 | 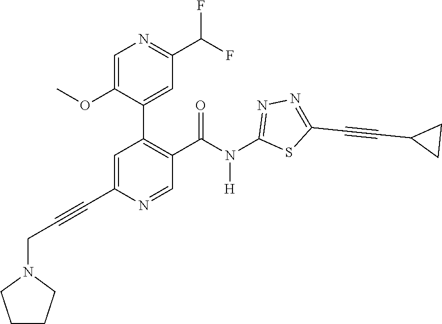 [SMILES: COc1cnc(C(F)F)cc1-c1cc(C#CCN2CCCC2)ncc1C(=O)Nc1nnc(C#CC2CC2)s1] |
| 601 | 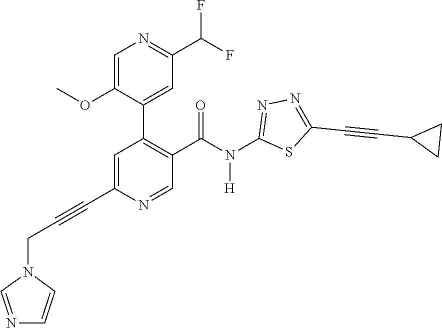 [SMILES: COc1cnc(C(F)F)cc1-c1cc(C#CCn2ccnc2)ncc1C(=O)Nc1nnc(C#CC2CC2)s1] |
| 602 | 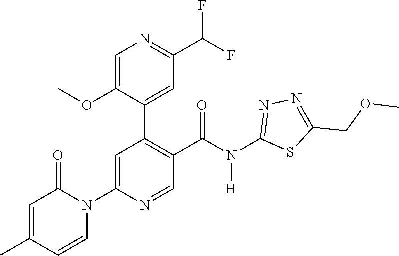 |
| 603 | 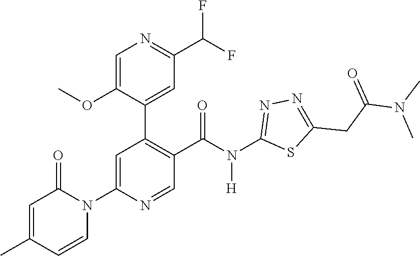 |
| 604 | 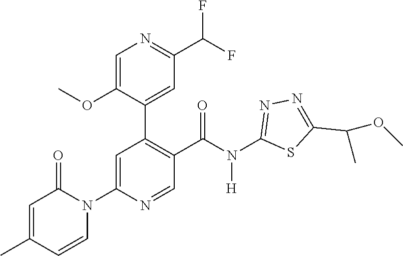 Racemic |
| 605 | 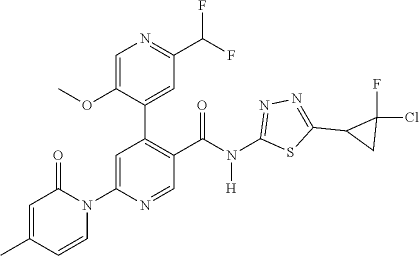 [SMILES: COc1cnc(C(F)F)cc1-c1cc(-n2ccc(C)cc2=O)ncc1C(=O)Nc1nnc(C2CC2(F)Cl)s1] Racemic |
| 606 | 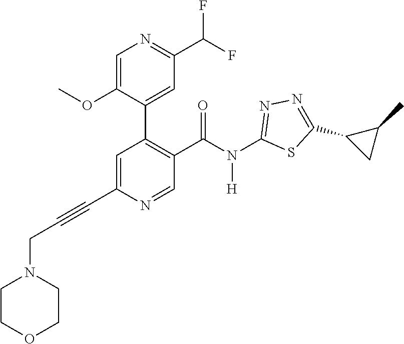 [SMILES: COc1cnc(C(F)F)cc1-c1cc(C#CCN2CCOCC2)ncc1C(=O)Nc1nnc([C@H]2C[C@@H]2C)s1] Racemic |
| 607 | 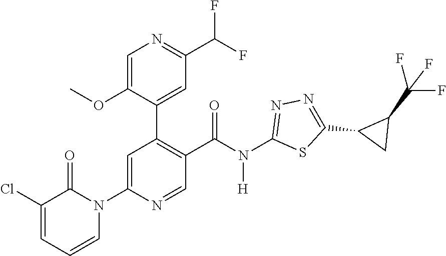 [SMILES: COc1cnc(C(F)F)cc1-c1cc(-n2cccc(Cl)c2=O)ncc1C(=O)Nc1nnc([C@H]2C[C@@H]2C(F)(F)F)s1] Chiral |
| 608 | 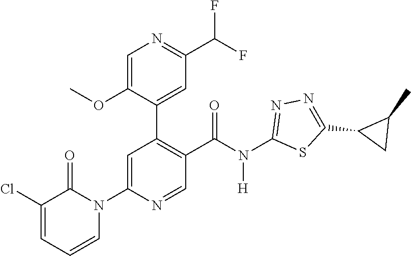 [SMILES: COc1cnc(C(F)F)cc1-c1cc(-n2cccc(Cl)c2=O)ncc1C(=O)Nc1nnc([C@H]2C[C@@H]2C)s1] Chiral |
| 609 | 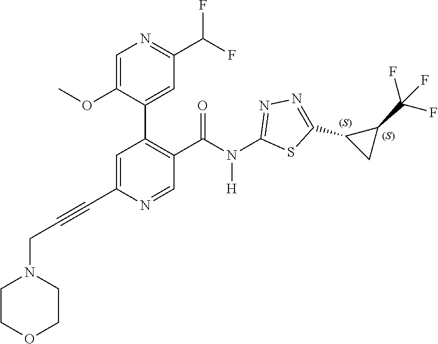 Chiral |
| 610 | 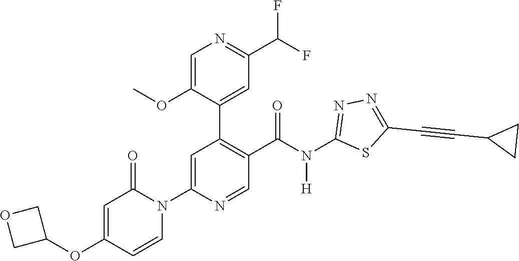 [SMILES: COc1cnc(C(F)F)cc1-c1cc(-n2ccc(OC3COC3)cc2=O)ncc1C(=O)Nc1nnc(C#CC2CC2)s1] |
| 611 | 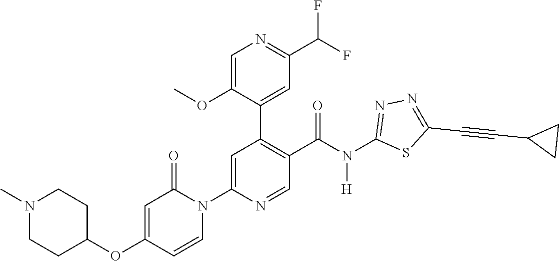 [SMILES: COc1cnc(C(F)F)cc1-c1cc(-n2ccc(OC3CCN(C)CC3)cc2=O)ncc1C(=O)Nc1nnc(C#CC2CC2)s1] |
| 612 | 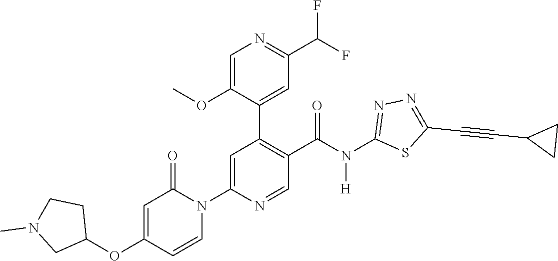 [SMILES: COc1cnc(C(F)F)cc1-c1cc(-n2ccc(OC3CCN(C)C3)cc2=O)ncc1C(=O)Nc1nnc(C#CC2CC2)s1] Racemic |
| 613 | 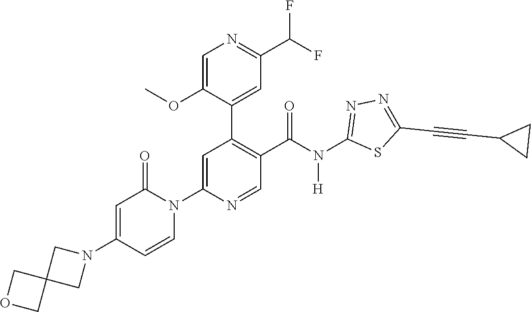 [SMILES: COc1cnc(C(F)F)cc1-c1cc(-n2ccc(N3CC4(COC4)C3)cc2=O)ncc1C(=O)Nc1nnc(C#CC2CC2)s1] |
| 614 | 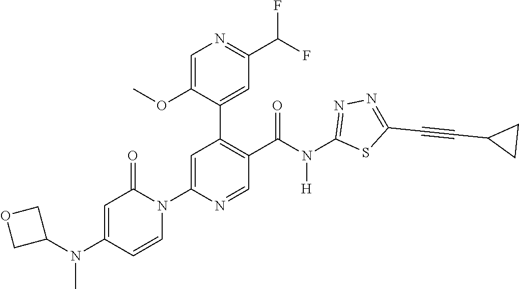 [SMILES: COc1cnc(C(F)F)cc1-c1cc(-n2ccc(N(C)C3COC3)cc2=O)ncc1C(=O)Nc1nnc(C#CC2CC2)s1] |
| 615 | 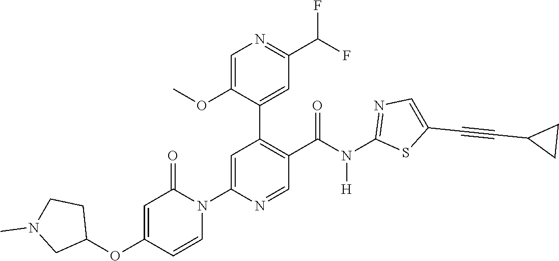 [SMILES: COc1cnc(C(F)F)cc1-c1cc(-n2ccc(OC3CCN(C)C3)cc2=O)ncc1C(=O)Nc1ncc(C#CC2CC2)s1] Racemic |
| 616 | 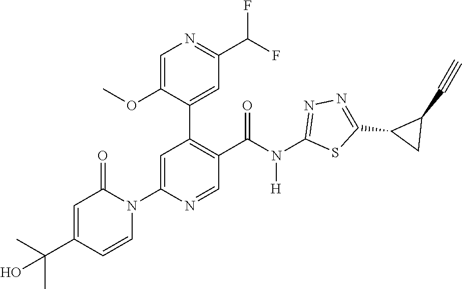 [SMILES: C#C[C@H]1C[C@@H]1c1nnc(NC(=O)c2cnc(-n3ccc(C(C)(C)O)cc3=O)cc2-c2cc(C(F)F)ncc2OC)s1] Chiral |
| 617 | 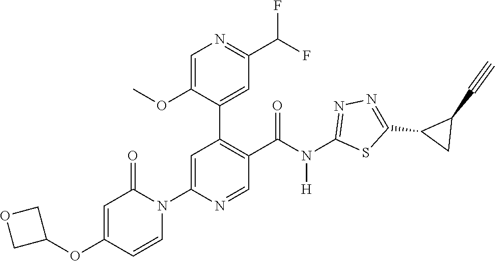 [SMILES: C#C[C@H]1C[C@@H]1c1nnc(NC(=O)c2cnc(-n3ccc(OC4COC4)cc3=O)cc2-c2cc(C(F)F)ncc2OC)s1] Chiral |
| 618 | 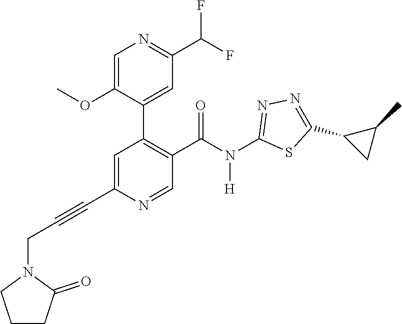 [SMILES: COc1cnc(C(F)F)cc1-c1cc(C#CCN2CCCC2=O)ncc1C(=O)Nc1nnc([C@H]2C[C@@H]2C)s1] Racemic |
| 619 | 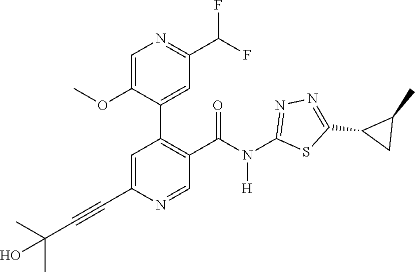 [SMILES: COc1cnc(C(F)F)cc1-c1cc(C#CC(C)(C)O)ncc1C(=O)Nc1nnc([C@H]2C[C@@H]2C)s1] Racemic |
| 620 | 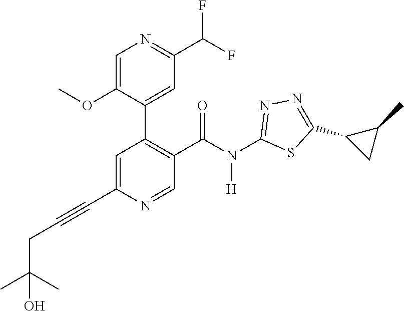 [SMILES: COc1cnc(C(F)F)cc1-c1cc(C#CCC(C)(C)O)ncc1C(=O)Nc1nnc([C@H]2C[C@@H]2C)s1] Racemic |
| 621 | 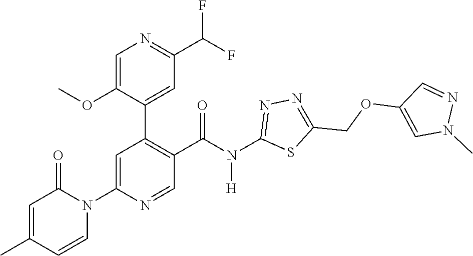 |
| 622 | 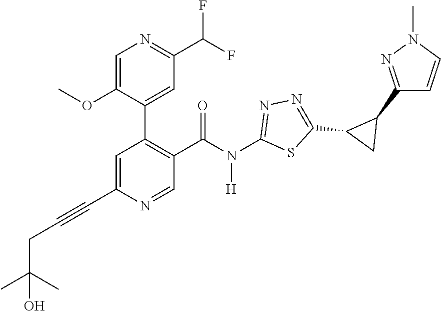 [SMILES: COc1cnc(C(F)F)cc1-c1cc(C#CCC(C)(C)O)ncc1C(=O)Nc1nnc([C@H]2C[C@@H]2c2ccn(C)n2)s1] Racemic |
| 623 | 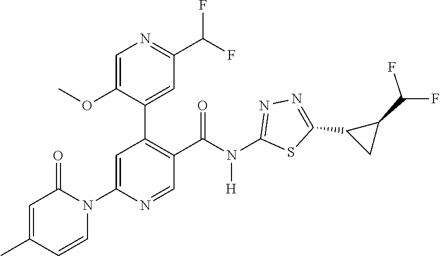 Chiral |
| 624 | 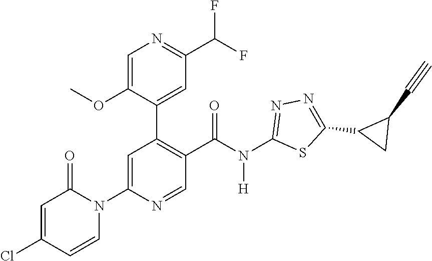 Chiral |
| 625 | 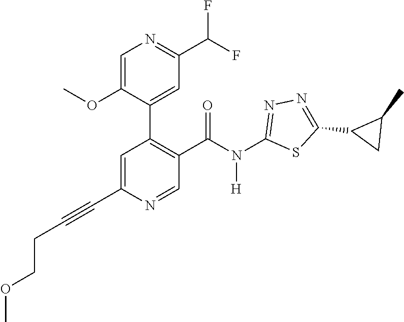 Racemic |
| 626 | 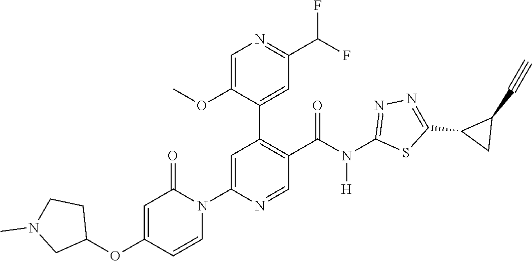 [SMILES: C#C[C@H]1C[C@@H]1c1nnc(NC(=O)c2cnc(-n3ccc(OC4CCN(C)C4)cc3=O)cc2-c2cc(C(F)F)ncc2OC)s1] Chiral |
| 627 | 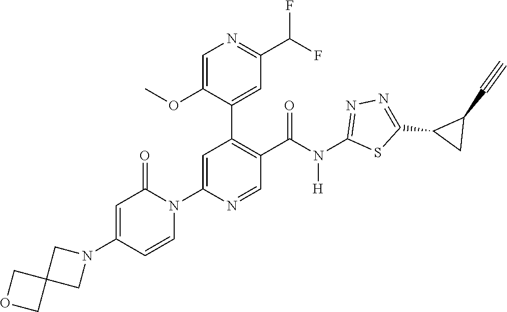 [SMILES: C#C[C@H]1C[C@@H]1c1nnc(NC(=O)c2cnc(-n3ccc(N4CC5(COC5)C4)cc3=O)cc2-c2cc(C(F)F)ncc2OC)s1] Chiral |
| 628 | 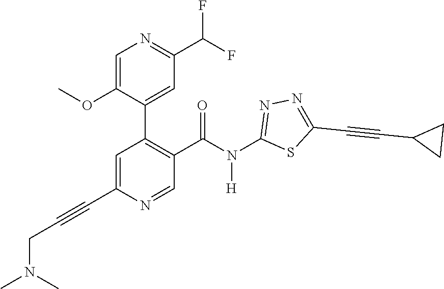 [SMILES: COc1cnc(C(F)F)cc1-c1cc(C#CCN(C)C)ncc1C(=O)Nc1nnc(C#CC2CC2)s1] |
| 629 | 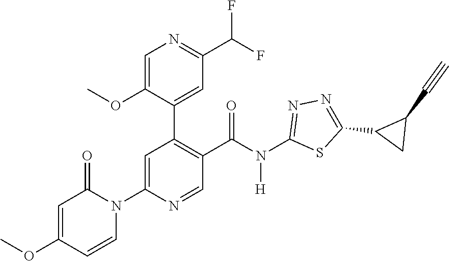 Chiral |
| 630 | 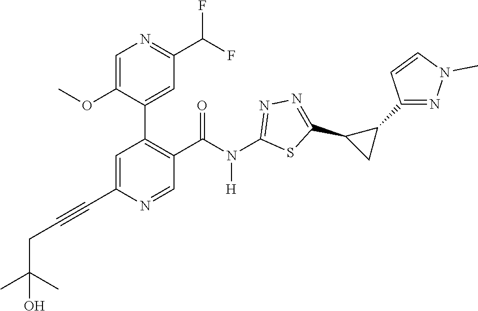 [SMILES: COc1cnc(C(F)F)cc1-c1cc(C#CCC(C)(C)O)ncc1C(=O)Nc1nnc([C@@H]2C[C@H]2c2ccn(C)n2)s1] Chiral |
| 631 | 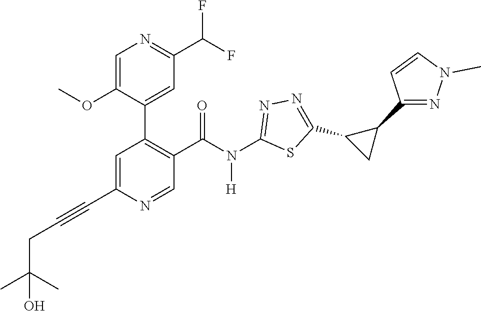 [SMILES: COc1cnc(C(F)F)cc1-c1cc(C#CCC(C)(C)O)ncc1C(=O)Nc1nnc([C@H]2C[C@@H]2c2ccn(C)n2)s1] Chiral |
| 632 | 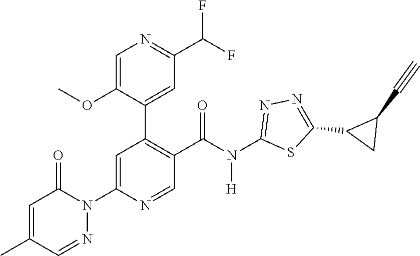 Chiral |
| 633 | 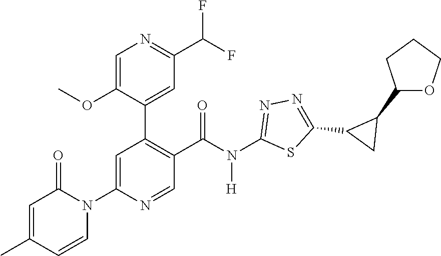 Racemic |
| 634 | 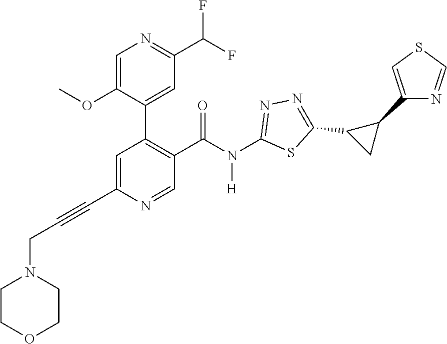 Racemic |
| 635 | 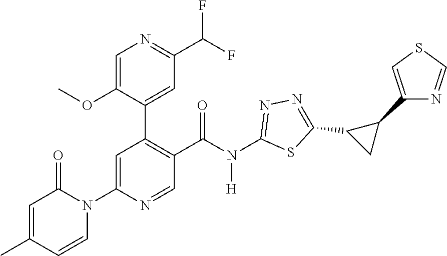 Racemic |
| 636 | 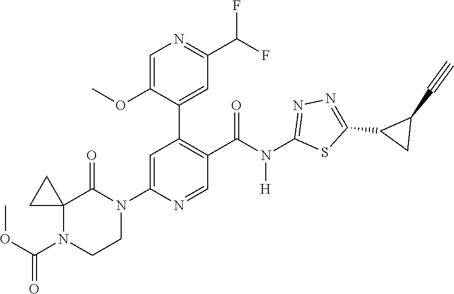 Chiral |
| 637 | 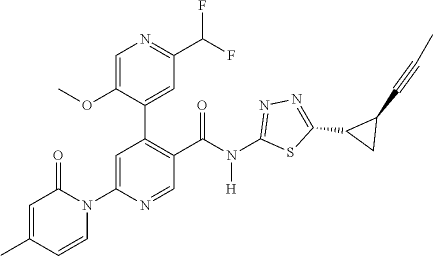 Racemic |
| 638 | 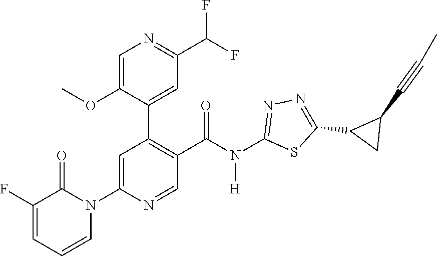 Racemic |
| 639 | 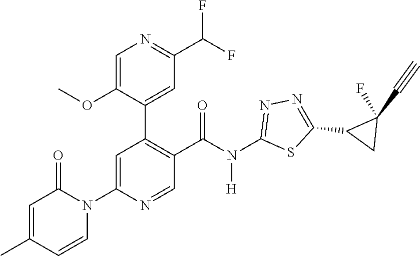 Racemic |
| 640 | 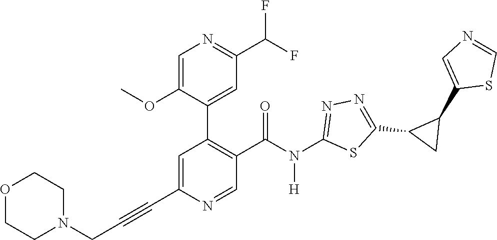 [SMILES: COc1cnc(C(F)F)cc1-c1cc(C#CCN2CCOCC2)ncc1C(=O)Nc1nnc([C@H]2C[C@@H]2c2cncs2)s1] Racemic |
| 641 | 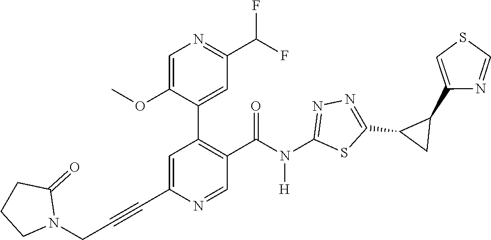 [SMILES: COc1cnc(C(F)F)cc1-c1cc(C#CCN2CCCC2=O)ncc1C(=O)Nc1nnc([C@H]2C[C@@H]2c2cscn2)s1] Racemic |
| 642 | 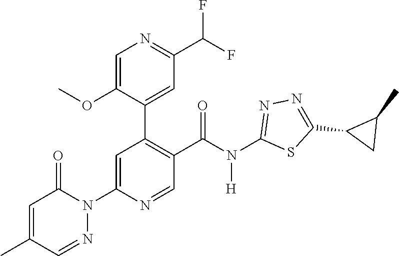 Chiral |
| 643 | 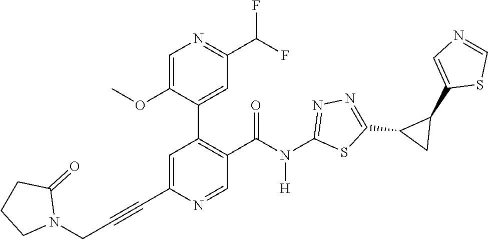 [SMILES: COc1cnc(C(F)F)cc1-c1cc(C#CCN2CCCC2=O)ncc1C(=O)Nc1nnc([C@H]2C[C@@H]2c2cncs2)s1] Racemic |

### Methods of Preparing a Compound of the Invention

Compounds of the invention may be prepared using reactions and techniques known in the art and those described herein.

### Method A

Compounds of Type III (Scheme 1) can be prepared by amide coupling of acids Type XI and amines of Type VII. The Type XI acids can by prepared in two steps by first, palladium cross-coupling reaction between aryl or heteroaryl bromo ester of Type IX and aryl or heteroaryl boronic acids of Type VIII, followed by saponification. The 1,3,4-thiadiazol-2-amine of Type VII are prepared by condensation of commercially available acids of Type VI with thiosemicarbazide in presence of POCl₃. Compound of Type III may alternatively be generated from the ester of Type X and 1,3,4-thiadiazol-2-amine of Type VII upon heating in presence of 1,5,7-triazabicyclo[4.4.0]dec-5-ene.

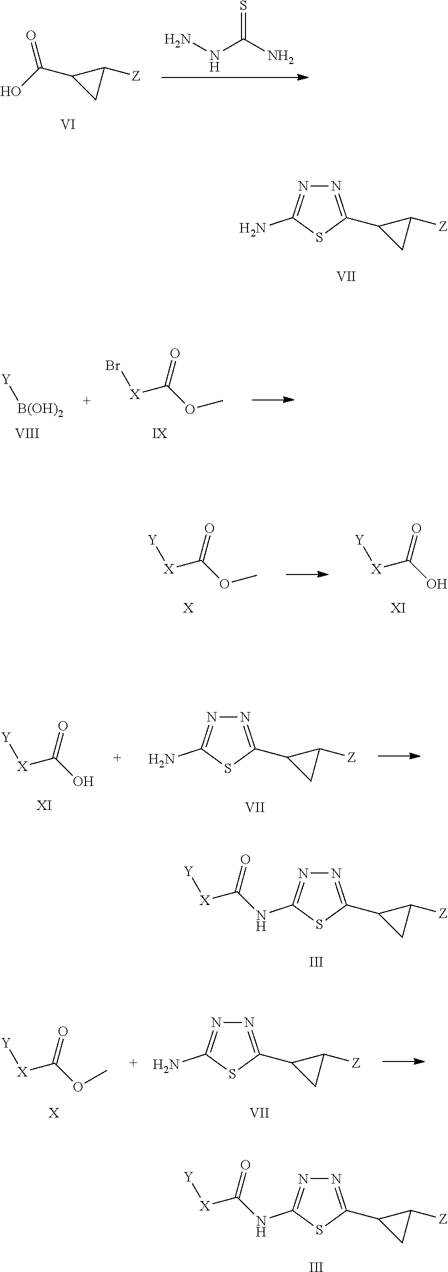

### Method B

Compounds of Type II (Scheme 2) can be prepared by a Sonogashira coupling between the Type XIII bromo thiadiazol and Type XIV alkynyl. Type XIII bromo thiadiazol can be prepared by the amide coupling between Type XI acid described previously and 5-bromo-1,3,4-thiadiazol-2-amine. Type XIV alkynyl can be prepared, if not commercially available, by Sonogashira coupling using the appropriate halogenated aryl or heteroaryl and ethynyltrimethylsilane followed by removal of the trimethylsilane protective group.

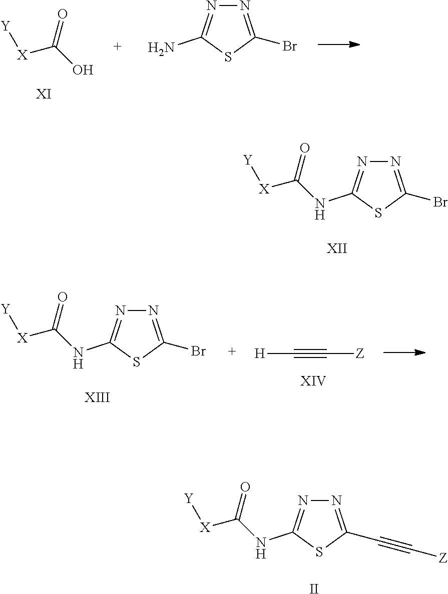

### Method C

Alternatively, Compound of Type II (Scheme 3) may be prepared by Sonogashira coupling between the alkynyl thiadiazol of Type XV and commercially available aryl or heteroaryl bromide of Type XVI. Alkynyl thiadiazol of Type XV can be obtained by amide coupling between acid of Type XI and 5-ethynyl-1,3,4-thiadiazol-2-amine. The latter is prepared in two steps starting from the Sonogashira coupling between Boc protected 5-bromo-1,3,4-thiadiazol-2-amine with ethynyltrimethylsilane followed by a one-pot double deprotection.

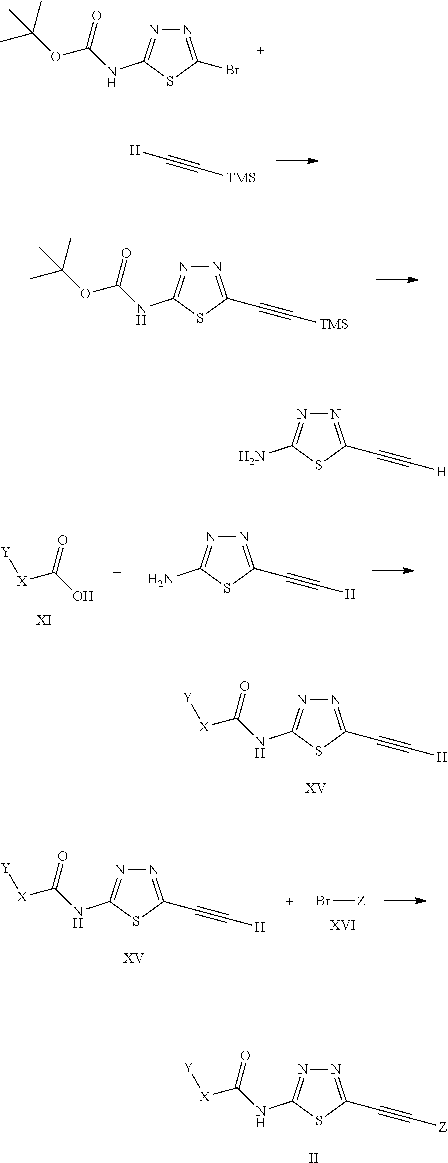

### Method D

Alternatively. Compound of Type II (Scheme 4) may also be prepared by amide coupling of previously described acid of Type XI and amino thiadiazol alkynyl of Type XVIII. The latter can be prepared in two steps by first Sonogashira coupling between tert-butyl (5-bromo-1,3,4-thiadiazol-2-yl) carbamate and previously described alkynyl of type XIV followed by removal of the Boc protecting group. Compound of Type II may alternatively be generated from the ester of Type X and amino thiadiazol alkynyl of Type XVIII upon heating in presence of 1,5,7-triazabicyclo[4.4.0]dec-5-ene.

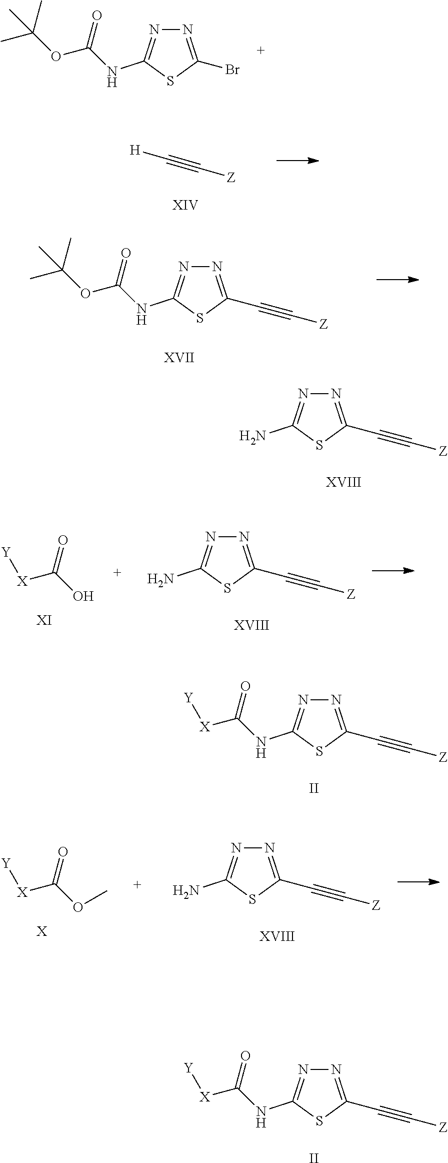

### Method E

Compound of Type IV (Scheme 5) may be prepared in a similar sequence of reactions described for compounds of Type II in Scheme 4. Amide coupling of previously described acid of Type XI and amino thiazole alkynyl of Type XX. The latter can be prepared in two steps by first Sonogashira coupling between tert-butyl (5-bromothiazol-2-yl)carbamate and previously described alkynyl of type XIV followed by removal of the Boc protecting group. Compound of Type IV may alternatively be generated from the ester of Type X and amino thiazole alkynyl of Type XX upon heating in presence of 1,5,7-triazabicyclo[4.4.0]dec-5-ene.

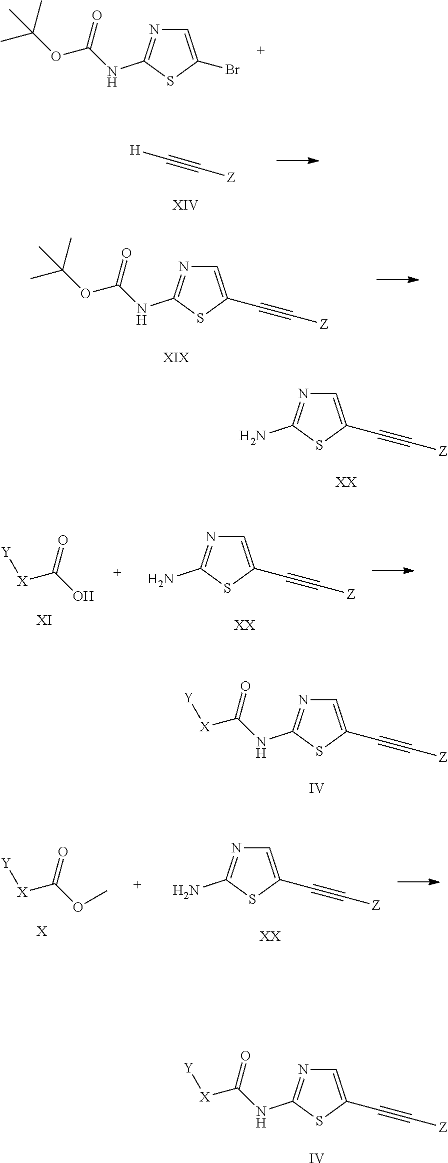

### Method F

Compound of Type XXII (Scheme 6) can be prepared in one step from the advanced 2-chloro pyridine intermediate of Type XXI by an SnAr type addition of a properly substituted amine (H—NR¹R²) in presence of a strong base. Similarly, compound of Type XXIII can be prepared using the same intermediate XXI by an SnAr type addition of a properly substituted alcohol (H—OR¹) in presence of a strong base.

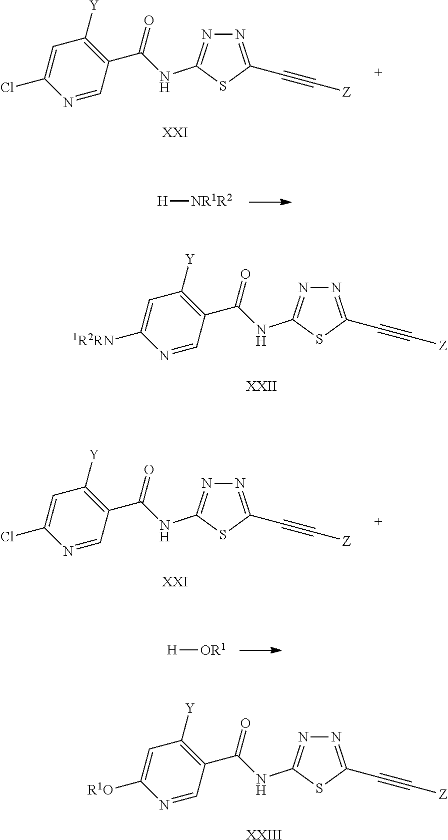

### Method G

Compound of Type XXIV (Scheme 7) can be prepared in one step from the advanced 2-chloro pyridine intermediate of Type XXI via a Sonogashira coupling with a properly substituted alkyne. Similarly, compound of Type XXVI can be prepared via a Sonogashira coupling using bromo phenyl intermediate XXV.

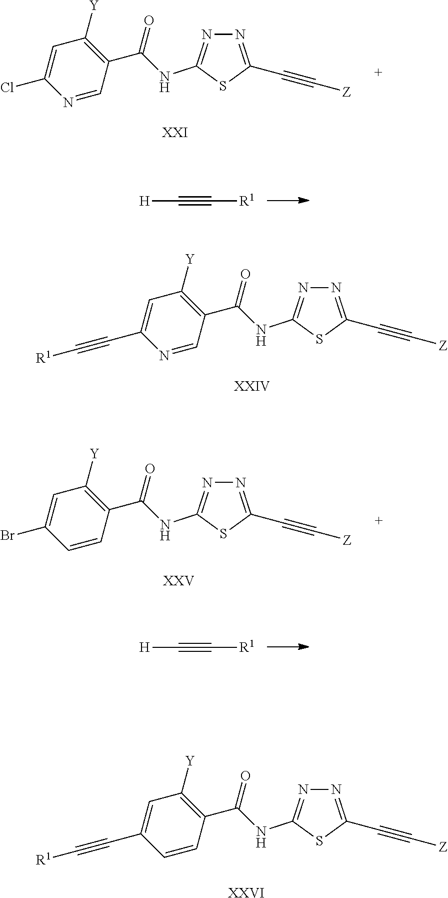

### Method H

Compounds of Type XXVIII (Scheme 8) can be prepared in one step from the advanced 2-chloro pyridine intermediate of Type XXVII via a Sonogashira coupling with a properly substituted alkyne. Compound of Type XXVII can be prepared via amide coupling of acids Type XI and amines of Type VII. Alternatively, a Sonogashira coupling can be performed first, with a properly substituted alkyne on the ester of Type XXIX to generate ester of Type XXX. Saponification and subsequent coupling with amines of Type VII, can provide compounds of Type XXVII.

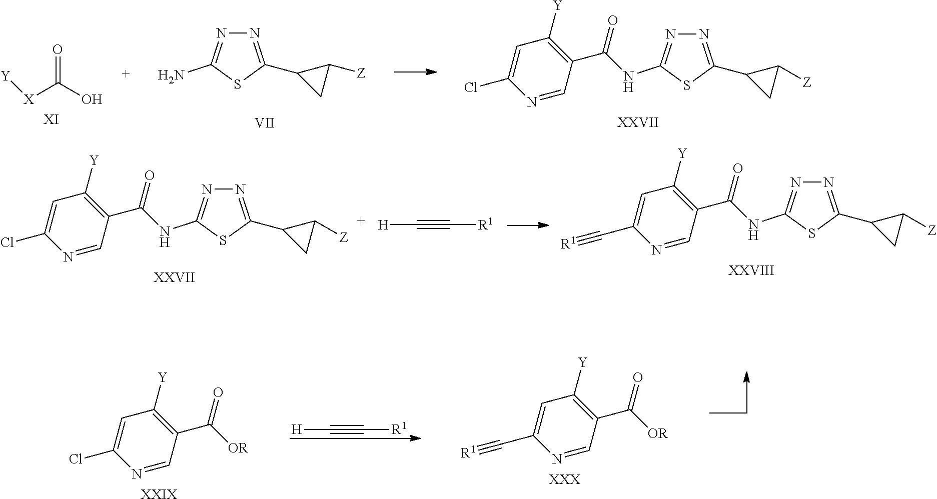

### Methods of Treatment

Compounds of the invention may be used for the treatment of a disease or condition mediated by Polθ in a subject by administering to the subject an effective amount of the compound of the invention.

The disease or condition may have the symptom of cell hyperproliferation. For example, the disease or condition may be a cancer. The cancer may be, e.g., carcinoma, sarcoma, adenocarcinoma, lymphoma, leukemia, or melanoma.

Non-limiting examples of cancers include prostate cancer, breast cancer, ovarian cancer, multiple myeloma, brain cancer, glioma, lung cancer, salivary cancer, stomach cancer, thymic epithelial cancer, thyroid cancer, leukemia, melanoma, lymphoma, gastric cancer, pancreatic cancer, kidney cancer, bladder cancer, colon cancer, and liver cancer.

Non-limiting examples of carcinomas include medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

Non-limiting examples of sarcomas include chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Non-limiting examples of leukemias include acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphoma, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

Non-limiting examples of melanomas include acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungual melanoma, and superficial spreading melanoma.

A compound of the invention may be administered by a route selected from the group consisting of oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, intratumoral, and topical administration.

The methods of the invention may include a step of identifying a subject as being a candidate for a Polθ Inhibitor therapy. For example, the subject may be identified as being a candidate for a Polθ inhibitor therapy by determining (i) whether the subject has cancer with defects in DNA repair; (ii) whether the subject has cancer, cancer cells, or cells expressing genetic aberrations in cancer-driving genes or oncogenes; (iii) whether the subject has cancer, cancer cell, or cells with one or more defect(s) in a protein or gene involved in DNA repair; (iv) whether the subject has cancer with defects in a protein or gene involved in homologous recombination; (v) whether the subject has a cancer with defects in a protein or gene that have been implicated in sensitivity to Polθ inhibitors or genetic perturbation of Polθ; or (vi) whether the subject has a cancer with genetic or protein characteristics that have been implicated in sensitivity to Polθ inhibitors.

The compounds, compositions, and methods described may be used to treat a subject having a cancer with an aberration in DNA repair. For example, the aberration in DNA repair may be, e.g., altered expression or activity of one or more of the following proteins/genes including but not limited to: BRCA2 and BRCA1. Aberrations in DNA repair may be identified by the presence of genomic scars reflective of use of microhomologies in DNA repair. Additionally, DNA repair may be identified as follows: 20% or greater change in RAD51 or gamma-H2AX foci.

The compounds, compositions, and methods described may be used to treat a subject having a cancer, cancer cells, or cells with one or more aberration(s) in DNA repair. For example, cancers which are homologous repair deficient by mechanisms other than BRCA deficiency, such as those with promoter hypermethylation. In these tumours where no DSB repair pathway may be fully down regulated the Polθ Inhibitor may be given along with another DNA damage response modulator such as a PARP inhibitor, a DNA-PK inhibitor, an ATM inhibitor, an ATR inhibitor, a wee1 inhibitor, a PKMYT1 inhibitor or a CHK1 inhibitor.

The compounds, compositions, and methods described may be used to treat a subject having a cancer, cancer cells or cells with one or more aberration(s) in a protein or gene involved in homologous recombination. For example, the aberration in homologous recombination may be altered expression or activity of one or more of the following proteins/genes including but not limited to: BRCA1, BRCA2, MRE11, RAD50, RAD51, RAD52, RAD54L, NBN, ATM, H2AX, PALB2, RPA, BRIP1, BARD1, ATR, ATRX, CHK1, CDK12, CHK2, MDM2, MDM4, FANCA, FANCC, FANCD2, FANCE, FANCF, FANCG, and FANCL.

The compounds, compositions, and methods described may be used to treat a subject having a cancer, cancer cells or cells with one or more aberration(s) in a protein or gene implicated in sensitivity to Polθ inhibitors or genetic perturbation of the Polθ signaling pathway including Polθ over-expression.

There are many methods known in the art for determining whether a tumor has an aberration in a protein or gene. For example, sequencing of either the genomic DNA or mRNA products of each specified gene (e.g., UNG, PARP1, or LIG1) can be performed on a sample of the tumor to establish whether mutations expected to modulate the function or expression of the gene product are present. In addition to the mutational inactivation, tumor cells can modulate a gene by hypermethylating its promoter region, leading to reduced gene expression. This is most commonly assessed using methylation-specific polymerase chain reaction (PCR) to quantify methylation levels on the promoters of base excision repair genes of interest. Analysis of DNA repair gene promoter methylation is available commercially.

The expression levels of genes can be assessed by directly quantifying levels of the mRNA and protein products of each gene using standard techniques, e.g., quantitative reverse transcriptase-coupled polymerase chain reaction (RT-PCR), RNA-Seq for gene expression, and immunohistochemistry (IHC) for protein expression. Gene amplification or deletion leading to aberrantly over- or under-expressed proteins (respectively) can also be measured by FISH (fluorescent in situ hybridization) analysis using a probe specific for the gene of interest.

The methods described above (gene sequence, promoter methylation, and mRNA expression) may also be used to characterize the status (e.g., expression or mutation) of other genes or proteins of interest, e.g., DNA-damaging oncogenes expressed by a tumor or defects in the DNA repair pathways of a cell.

### PARP Inhibitors

PARP inhibitors that may be used in the present invention include compounds that upon contacting PARP, whether in vitro, in cell culture, in an animal or in a patient, reduce the activity of PARP, such that the measured PARP IC₅₀ is 10 μM or less (e.g., 5 μM or less or 1 μM or less). For certain PARP inhibitors, the PARP IC₅₀ may be 100 nM or less (e.g., 10 nM or less, or 1 nM or less) and could be as low as 100 pM or 10 pM. Preferably, the PARP IC₅₀ is 0.1 nM to 1 μM (e.g., 0.1 nM to 750 nM, 0.1 nM to 500 nM, or 0.1 nM to 250 nM). For example, certain PARP inhibitors may be prepared using techniques and methods disclosed in, e.g., International Application No. PCT/US2022/025357, which is incorporated by reference herein.

PARP Inhibitors include:

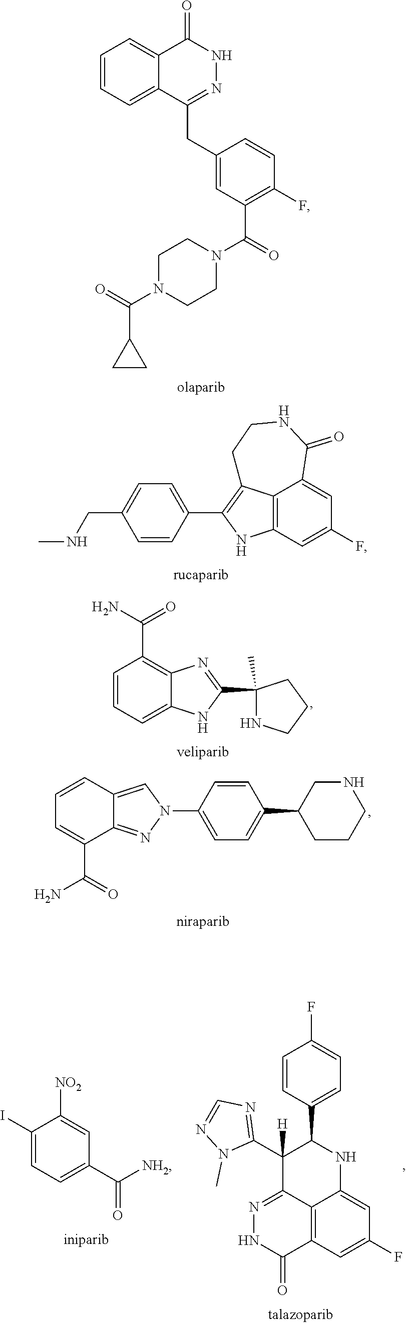 [SMILES: O=C(c1cc(Cc2n[nH]c(=O)c3ccccc23)ccc1F)N1CCN(C(=O)C2CC2)CC1 ; CNCc1ccc(-c2[nH]c3cc(F)cc4c3c2CCNC4=O)cc1 ; C[C@]1(c2nc3c(C(N)=O)cccc3[nH]2)CCCN1 ; NC(=O)c1cccc2cn(-c3ccc([C@@H]4CCCNC4)cc3)nc12 ; NC(=O)c1ccc(I)c([N+](=O)[O-])c1 ; Cn1ncnc1[C@H]1c2n[nH]c(=O)c3cc(F)cc(c23)N[C@@H]1c1ccc(F)cc1]

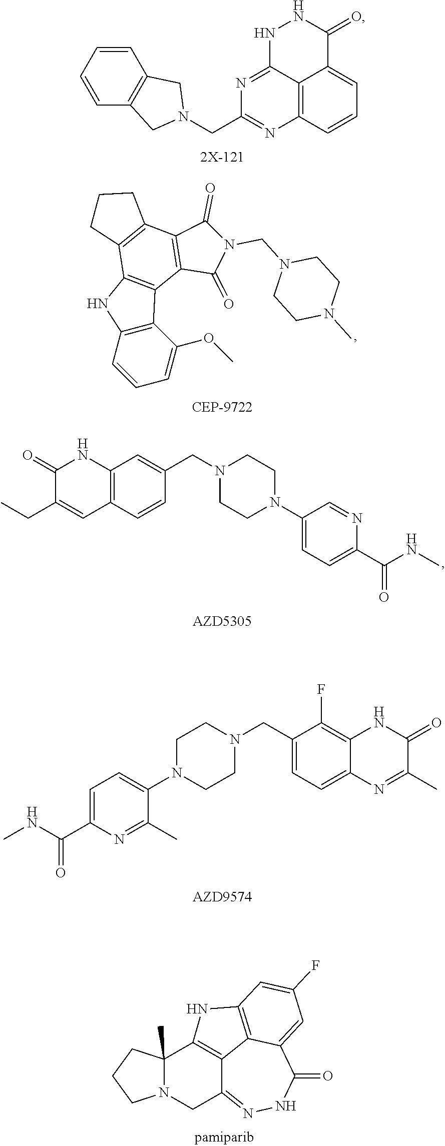

and pharmaceutically acceptable salts thereof.

Non-limiting examples of PARP inhibitors include those described in PCT applications PCT/CN2022/086311, PCT/CN2022/115259, PCT/US2022/027334, PCT/CN2022/088989, and PCT/CN2022/087969 and U.S. Pat. Nos. 11,325,906, 8,716,493, 8,236,802, 8,071,623, 8,012,976, 7,732,491, 7,550,603, 7,531,530, 7,151,102, and 6,495,541, each of which is incorporated herein by reference herein. A PARP inhibitor may be isotopically enriched (e.g., enriched for deuterium).

### DNA-Dependent Protein Kinase Inhibitors

DNA-dependent Protein Kinase (DNA-PK) inhibitors that may be used in the present invention include compounds that upon contacting DNA-PK, whether in vitro, in cell culture, or in an animal, reduce the activity of DNA-PK, such that the measured DNA-PK IC₅₀ is 10 μM or less (e.g., 5 μM or less or 1 μM or less). For certain DNA-PK inhibitors, the DNA-PK IC₅₀ may be 100 nM or less (e.g., 10 nM or less, or 1 nM or less) and could be as low as 100 μM or 10 μM. Preferably, the DNA-PK IC₅₀ is 0.1 nM to 1 μM (e.g., 0.1 nM to 750 nM, 0.1 nM to 500 nM, or 0.1 nM to 250 nM).

DNA-PK inhibitors include AZD-7648, Peposertib, M9831, IMP11, NU5455, BAY-8400, ZL-2201, adMare Bioinnovations DNA-PK Program, XRD-0394, Avadomide, NERx Ku program, CC-115, KU57788, ZSTK474, LY3023414, BR101801, XRD-0394 and NK-314.

### Antibody Drug Conjugates

Antibody drug conjugates (ADCs) that may be used in the present invention include conjugates that upon contacting cancer cells, whether in vitro, in cell culture, or in an animal, inhibit the cancer cell, such that the measured IC₅₀ is 10 μM or less (e.g., 5 μM or less or 1 μM or less). For certain ADCs, the IC₅₀ may be 100 nM or less (e.g., 10 nM or less, or 1 nM or less) and could be as low as 100 pM or 10 pM. Preferably, the ADC IC₅₀ is 0.1 nM to 1 μM (e.g., 0.1 nM to 750 nM, 0.1 nM to 500 nM, or 0.1 nM to 250 nM).

ADCs include Disitamab vedotin, Belantamab mafodotin, Trastuzumab deruxtecan, Ujvira, Mirvetuximab soravtansine, Gemtuzumab ozogamicin, Enfortumab vedotin, Inotuzumab ozogamicin, Trastuzumab emtansine, Tisotumab vedotin, Sacituzumab govitecan, Polatuzumab vedotin, Loncastuximab Tesirine, Brentuximab vedotin, PF-06804103, MGTA-117, FOR46, MRG001, SOT102, ZV0203, AOC 1020, PRO1184, BAT8009, BB-1705, JS107, SHR-A1912, CMG901, Ladiratuzumab vedotin, BAT8006, RC108, BAT8008, Mipasetamab Uzoptirine, NBE-002, Zanidatamab zovodotin, F0002-ADC, SKB315, G01001, ABBV-637, XMT-2056, TORL-1-23, FDA022, DYNE-251, STI-6129, Ozuriftamab vedotin, Farletuzumab Ecteribulin, Trastuzumab vedotin, DB-1303, OMTX705, TRS005, Ispectamab debotansine, DXC-005, ESG-401, ARX788, BAT8010, Tusamitamab ravtansine, ABBV-154, Naratuximab emtansine, PSMA ADC, TAK-164, ADCT-602, ADCT-901, SHR-A1201, GB251, ABL202, SHR-A1921, 9MW2821, HS-20093, BIO-106, SKB264, Camidanlumab Tesirine, Datopotamab deruxtecan, Telisotuzumab vedotin, L-DOS47, AVID100, OBI-999, DP303c. AURIXIM, MT-8633, IMGC936, BB-1701, AOC 1001, JS108, TAC-001, SYSA1801, SHR-A2009, TORL-2-307-ADC, BL-M07D1, STRO-001, A166. Mecbotamab vedotin, Trastuzumab duocarmazine, ASN004, ABBV-011, Mirzotamab clezutoclax, OBT076, HS630, SGN-STNV, FDA018, ABBV-400, AZD8205, IBI-343, SGN-ALPV, TAK-500, JBH492, ALT-P7, Ifinatamab deruxtecan, DXC-004, IMGN151, XMT-1660, M1231, LM-102, ORM-5029, STI-3258, SGN-B7H4V, TPX-4589, IKS03, Zilovertamab Vedotin, ARX517, Pivekimab Sunirine, Lonigutamab Ugodotin, TRPH-222, MRG004a, DS-6000a, REGN5093-M114, Trastuzumab imbotolimod, RC88. HTI-1066, BI-CON-02, SGN-CD228A, AOC 1044, DB-1305, ABBV-319, Patritumab Deruxtecan, RC118, Trastuzumab rezetecan, ARX305, Upifitamab Rilsodotin, NBT828, TAA013. BL-B01D1, BL-M02D1, G01007, DS-9606a, NBT508, B003, DX126-262, XB002, FS-1502, Praluzatamab ravtansine, AMT-151, M9140, Indatuximab ravtansine, Cofetuzumab pelidotin, RG7861, AGS62P1, CX-2029, SGN-B6A, Unspecified TROP2 ADC, Unspecified HER2 ADC, RC98 ADC, DYNE-101, SHR-A1904, Anetumab ravtansine, Vobramitamab duocarmazine, Luveltamab tazevibulin, Serclutamab talirine, MRG003, SYD1875, BYON3521, SGN-PDL1V, JSKN-003, YL201, HS-20089, DXC-007, SYS6002, and HDP-101.

### Radiotherapy

Radiotherapy may be used in conjunction with the other methods of this disclosure for the treatment of cancer. Radiotherapy is a treatment method where ionizing radiation is provided to cancerous tissue to induce DNA damage and cell death. Radiotherapy methods of this disclosure include radiation from an external beam (i.e., external beam radiation), sealed source radiotherapy, (i.e., brachytherapy), and injection of radionuclide isotopes (i.e., radionuclide therapy) or radioligand therapy (RLT) in which pharmaceuticals comprising antibodies or cell surface ligands are linked to radionuclides for administration. External beam radiation can include treatment with photons (X-rays), electrons, protons, carbon ions, boron capture neutrons, etc. Radionuclides used in brachytherapy, radionuclide therapy, and radioligand therapy can include: ¹³¹I, ⁷⁷Lu, ¹⁵Sm, ⁹⁰Y, ²²³Ra, ²²⁵Ac, ²¹¹At, ²¹³Bi, ²¹²Pb/²¹²Bi, ¹⁶¹Th, ¹²⁵I, ¹³¹Cs, ¹⁰⁶Ru, ¹⁰³Pd, ³²P, ³³P, ⁶⁷Cu, ⁸⁹Sr, ¹⁶⁵Dy, ¹⁶⁶Ho, ¹⁸⁶Re, ¹⁸³Re, ⁶⁰Co, etc.

In some embodiments, the radiotherapy is part of a chemoradiotherapy (CRT). The chemotherapeutic agent can be etoposide, doxorubicin, topotecan, Irinotecan, fluorouracil, gemcitabine, paclitaxel, a platin, an anthracycline, and a combination thereof.

The radiotherapy can be a treatment given with electrons, photons, protons, alpha-emitters, beta-emitters, other ions, radio-nucleotides, boron capture neutrons, and combinations thereof.

In some embodiments, the radiotherapy is given either with fractionation (e.g., 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.00, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, and 2.00 Gy per day for 5 days a week) up to a total dose of 50-70 Gy. In some embodiments, the radiotherapy is given either with fractionation (0.1 to 2 Gy per day for 5 days a week) up to a total dose of 50-70 Gy. Other fractionation schedules could also be envisioned, for example, a lower dose per fraction but given twice daily. Higher daily doses over a shorter period of time can also be given. In one embodiment, stereotactic radiotherapy as well as the gamma knife are used. In the palliative setting, other fractionation schedules are also widely used for example 25 Gy in 5 fractions or 30 Gy in 10 fractions. For radiotherapy, the duration of treatment will be the time frame when radiotherapy is given. These interventions apply to treatment given with electrons, photons and protons, alfa-emitters or other ions, treatment with radio-nucleotides, for example, treatment with ¹³¹I given to patients with thyroid cancer, as well in patients treated with boron capture neutron therapy.

### Radiotherapy Ligands

Radiotherapy Ligands (RLTs) that may be used in the present invention include agents that upon contacting cancer cells, whether in vitro, in cell culture, or in an animal, inhibit the cancer cell, such that the measured IC₅₀ is 10 μM or less (e.g., 5 μM or less or 1 μM or less). For certain RLTs, the IC₅₀ may be 100 nM or less (e.g., 10 nM or less, or 1 nM or less) and could be as low as 100 pM or 10 pM. Preferably, the RLT IC₅₀ is 0.1 nM to 1 μM (e.g., 0.1 nM to 750 nM, 0.1 nM to 500 nM, or 0.1 nM to 250 nM).

RLTs include Zevalin, Actimab-A, lomab-ACT, lomab-B, Lutetium-177-DOTAGA-PEG-IAC, Tozaride. SS0110, BAY-2701439, 177Lu-rhPSMA-10.1, CTT-1403, Iopofosine, SAR-BBN, SAR-bisPSMA, SARTATE, FAP-2286, CONV-01-α, ¹¹⁷Lu-PSMA-I&T, FPI-2059, FPI-1434, FPI-1966, [¹¹⁷Lu] ludotadipep, ¹⁸¹Tb-PSMA-I&T, ITM-31, ITM-11, JNJ-69086420, I-¹³¹-1095, Azedra, PSMA TTC/BAY-2315497, ¹⁷⁷Lu-DOTA-EB-TATE, Betalutin, AAA817, AAA603, Lutathera, Pluvicto, PPMX-T002, 186RNL, PNT2003, CAM−H2, AlphaMedix, RYZ101, Sn-¹¹⁷m-DTPA, TLX592, TLX66, TLX250, TLX591, TLX101, ¹²⁴I-omburtamab, GD2-SADA, and ¹³¹I-omburtamab.

### Immune Checkpoint Inhibitors

Immune checkpoint inhibitors reinvigorate antitumor immune responses by interrupting co-Inhibitory signaling pathways and promote immune-mediated elimination of tumor cells which can involve DNA damage or recognition of DNA damage. Immune checkpoint inhibitors that may be used in the present invention include compounds that upon contacting a cell, whether in vitro, in cell culture, in an animal, or in a patient, block an immune checkpoint. Immune checkpoint inhibitors include: anti CTLA-4 Ipilimumab (Yervoy), anti PD-1 Nivolumab (Opdivo), anti PD-1 Pembrolizumab (KEYTRUDA), anti PD-1 Cemiplimab (LIBTAYO), anti PD-L1 Atezolizumab (TECENTRIQ), anti PD-L1 Avelumab (BAVENCIO), and anti PD-L1 Durvalumab (IMFINZI).

### Pharmaceutical Compositions

The compounds used in the methods described herein are preferably formulated into pharmaceutical compositions for administration to human subjects in a biologically compatible form suitable for administration in vivo. Pharmaceutical compositions typically include a compound as described herein and a pharmaceutically acceptable excipient. Certain pharmaceutical compositions may include one or more additional pharmaceutically active agents described herein.

The compounds described herein can also be used in the form of the free base, in the form of salts, zwitterions, solvates, or as prodrugs, or pharmaceutical compositions thereof. All forms are within the scope of the invention. The compounds, salts, zwitterions, solvates, prodrugs, or pharmaceutical compositions thereof, may be administered to a patient in a variety of forms depending on the selected route of administration, as will be understood by those skilled in the art. The compounds used in the methods described herein may be administered, for example, by oral, parenteral, buccal, sublingual, nasal, rectal, patch, pump, or transdermal administration, and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

For human use, a compound of the invention can be administered alone or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of a compound of the invention into preparations which can be used pharmaceutically.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, e.g., preservatives.

The excipient or carrier is selected based on the mode and route of administration. Suitable pharmaceutical carriers, as well as pharmaceutical necessities for use in pharmaceutical formulations, are described in Remington: The Science and Practice of Pharmacy, 21st Ed., Gennaro, Ed., Lippincott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents. e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6th Edition, Rowe et al., Eds., Pharmaceutical Press (2009).

These pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21st Ed., Gennaro, Ed., Lippincott Williams & Wilkins (2005), and Encyclopedia of Pharmaceutical Technology, eds. J. Swarbrick and J. C.

Boylan, 1988-1999, Marcel Dekker, New York. Proper formulation is dependent upon the route of administration chosen. The formulation and preparation of such compositions is well-known to those skilled in the art of pharmaceutical formulation. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

### Dosages

The dosage of the compound used in the methods described herein, or pharmaceutically acceptable salts or prodrugs thereof, or pharmaceutical compositions thereof, can vary depending on many factors, e.g., the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds used in the methods described herein may be administered initially in a suitable dosage that may be adjusted as required, depending on the clinical response. In general, a suitable daily dose of a compound of the invention will be that amount of the compound that is the lowest dose effective to produce a therapeutic effect. Such an effective dose will generally depend upon the factors described above.

A compound of the invention may be administered to the patient in a single dose or in multiple doses. When multiple doses are administered, the doses may be separated from one another by, for example, 1-24 hours, 1-7 days, 1-4 weeks, or 1-12 months. The compound may be administered according to a schedule, or the compound may be administered without a predetermined schedule. An active compound may be administered, for example, 1, 2, 3, 4, 5, 6, 7, 8, 9, 10, 11, or 12 times per day, every 2nd, 3rd, 4th, 5th, or 6th day, 1, 2, 3, 4, 5, 6, or 7 times per week, 1, 2, 3, 4, 5, or 6 times per month, or 1, 2, 3, 4, 5, 6, 7, 8, 9, 10, 11, or 12 times per year. It is to be understood that, for any particular subject, specific dosage regimes should be adjusted over time according to the individual need and the professional judgment of the person administering or supervising the administration of the compositions.

While the attending physician ultimately will decide the appropriate amount and dosage regimen, an effective amount of a compound of the invention may be, for example, a total daily dosage of, e.g., between 0.05 mg and 3000 mg of any of the compounds described herein. Alternatively, the dosage amount can be calculated using the body weight of the patient. Such dose ranges may include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the compound is administered.

In the methods of the invention, the time period during which multiple doses of a compound of the invention are administered to a patient can vary. For example, in some embodiments, doses of the compounds of the invention are administered to a patient over a time period that is 1-7 days; 1-12 weeks; or 1-3 months. In other embodiments, the compounds are administered to the patient over a time period that is, for example, 4-11 months or 1-30 years. In other embodiments, the compounds are administered to a patient at the onset of symptoms. In any of these embodiments, the amount of compound that is administered may vary during the time period of administration. When a compound is administered daily, administration may occur, for example, 1, 2, 3, 4, 5, 6, 7, 8, 9, 10, 11, or 12 times per day.

### Formulations

A compound identified as capable of treating any of the conditions described herein, using any of the methods described herein, may be administered to patients or animals with a pharmaceutically acceptable diluent, carrier, or excipient, in unit dosage form. The chemical compounds for use in such therapies may be produced and isolated by any standard technique known to those in the field of medicinal chemistry. Conventional pharmaceutical practice may be employed to provide suitable formulations or compositions to administer the identified compound to patients suffering from a bacterial infection. Administration may begin before the patient is symptomatic.

Exemplary routes of administration of the compounds (e.g., a compound of the invention), or pharmaceutical compositions thereof, used in the present invention include oral, sublingual, buccal, transdermal, intradermal, Intramuscular, parenteral, intravenous, intra-arterial, intracranial, subcutaneous, intraorbital, intraventricular, intraspinal, intraperitoneal, intranasal, Inhalation, and topical administration. The compounds desirably are administered with a pharmaceutically acceptable carrier. Pharmaceutical formulations of the compounds described herein formulated for treatment of the disorders described herein are also part of the present invention.

### Formulations for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Stabilized amorphous formulations may also be used for oral administration. A technique such as spray-dried dispersion may used in which the active drug is mixed with a polymer such as a cellulose derivative (e.g., cellulose acetate phthalate (CAP), methylcellulose acetate phthalate, hydroxypropylmethyl cellulose (HPMC), and hydroxypropylmethyl cellulose acetate succinate (HPMCAS, e.g., HPMCAS grade H, HPMCAS grade L, and HPMCAS grade M)), a polyacrylate (e.g., polymethacrylate, a methacrylate copolymer, and an ethacrylate copolymer), a polyvinyl pyrrolidone, a polyvinyl acetate (e.g., polyvinyl acetate ester and a polyethylene glycol-polyvinylcaprolactam-polyvinylacetate copolymer), or a copolymer of a polyvinyl pyrrolidone and a polyvinyl acetate, and combinations thereof dissolved in an organic solvent. The resulting solution may be rapidly dried by a stream of air in a spray drying apparatus to produce a fine powder containing the active drug as an amorphous solid. Alternatively, the active drug may be dissolved in a polymer carrier such as povidone, hydroxypropyl methylcellulose, microcrystalline cellulose, or a mixture of such agents, using a hot melt extrusion process to generate an amorphous solid.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, di-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

### Formulations for Parenteral Administration

The compounds described herein for use in the methods of the invention can be administered in a pharmaceutically acceptable parenteral (e.g., intravenous or intramuscular) formulation as described herein. The pharmaceutical formulation may also be administered parenterally (intravenous, intramuscular, subcutaneous or the like) in dosage forms or formulations containing conventional, non-toxic pharmaceutically acceptable carriers and adjuvants. In particular, formulations suitable for parenteral administration include aqueous and non-aqueous sterile injection solutions which may contain anti-oxidants, buffers, bacterlostats and solutes which render the formulation isotonic with the blood of the intended recipient; and aqueous and non-aqueous sterile suspensions which may include suspending agents and thickening agents. For example, to prepare such a composition, the compounds of the invention may be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl, or n-propyl p-hydroxybenzoate. Additional information regarding parenteral formulations can be found, for example, in the United States Pharmacopeia-National Formulary (USP-NF), herein incorporated by reference.

The parenteral formulation can be any of the five general types of preparations identified by the USP-NF as suitable for parenteral administration:
(1) “Drug Injection:” a liquid preparation that is a drug substance (e.g., a compound of the invention), or a solution thereof;
(2) “Drug for Injection:” the drug substance (e.g., a compound of the invention) as a dry solid that will be combined with the appropriate sterile vehicle for parenteral administration as a drug injection;
(3) “Drug Injectable Emulsion:” a liquid preparation of the drug substance (e.g., a compound of the invention) that is dissolved or dispersed in a suitable emulsion medium;
(4) “Drug Injectable Suspension:” a liquid preparation of the drug substance (e.g., a compound of the invention) suspended in a suitable liquid medium; and
(5) “Drug for Injectable Suspension:” the drug substance (e.g., a compound of the invention) as a dry solid that will be combined with the appropriate sterile vehicle for parenteral administration as a drug injectable suspension.

Formulations for parenteral administration include solutions of the compound prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 21st Ed., Gennaro, Ed., Lippincott Williams & Wilkins (2005) and in The United States Pharmacopeia: The National Formulary (USP 36 NF31), published in 2013.

Formulations for parenteral administration may, for example, contain excipients, sterile water, or saline, polyalkylene glycols, e.g., polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the compound. Exemplary formulations for parenteral release of the compound include: aqueous solutions, powders for reconstitution, cosolvent solutions, oil/water emulsions, suspensions, oil-based solutions, liposomes, microspheres, and polymeric gels.

### Combinations

Compounds of the present invention may be administered to a subject in combination with a one or more additional agents, e.g.:
(a) a cytotoxic agent;
(b) an antimetabolite;
(c) an alkylating agent;
(d) an anthracycline;
(e) an antibiotic;
(f) an anti-mitotic agent;
(g) a hormone therapy;
(h) a signal transduction inhibitor;
(i) a gene expression modulator;
(j) an apoptosis inducer;
(k) an angiogenesis inhibitor;
(l) an immunotherapy agent;
(m) a DNA damage repair inhibitor;
(n) a kinase inhibitor
(o) a PARP inhibitor
(p) an ionizing radiation therapy
(q) a radioligand therapy
(r) antibody drug conjugate (ADC)
or
a combination thereof.

The cytotoxic agent may be, e.g., actinomycin-D, alemtuzumab, alitretinoin, allopurinol, altretamine, amifostine, amphotericin, amsacrine, arsenic trioxide, asparaginase, azacitidine, azathioprine, Bacille Calmette-Guérin (BCG), bendamustine, bexarotene, bevacuzimab, bleomycin, bortezomib, busulphan, capecitabine, carboplatin, carfilzomib, carmustine, cetuximab, cisplatin, chlorambucil, cladribine, clofarabine, colchicine, crisantaspase, cyclophosphamide, cyclosporine, cytarabine, cytochalasin B, dacarbazine, dactinomycin, darbepoetin alfa, dasatinib, daunorubicin, 1-dehydrotestosterone, denileukin, dexamethasone, dexrazoxane, dihydroxy anthracin dione, disulfiram, docetaxel, doxorubicin, emetine, epirubicin, erlotinib, epigallocatechin gallate, epoetin alfa, estramustine, ethidium bromide, etoposide, everolimus, filgrastim, finasunate, floxuridine, fludarabine, flurouracil (5-FU), fulvestrant, ganciclovir, geldanamycin, gemcitabine, glucocorticoids, gramicidin D, histrelin acetate, hydroxyurea, Ibritumomab, Idarubicin, ifosfamide, imatinib, Irinotecan, interferons. Interferon alfa-2a, interferon alfa-2b, ixabepilone, lactate dehydrogenase A (LDH-A), lenalidomide, letrozole, leucovorin, levamisole, lidocaine, lomustine, mechlorethamine, melphalan, 6-mercaptopurine, mesna, methotrexate, methoxsalen, metoprine, metronidazole, mithramycin, mitomycin-C, mitoxantrone, nandrolone, nelarabine, nilotinib, nofetumomab, oprelvekin, oxaliplatin, paclitaxel, pemetrexed, pentostatin, palifermin, pamidronate, pegademase, pegaspargase, pegfilgrastim, pemetrexed disodium, plicamycin, porfimer sodium, procaine, procarbazine, propranolol, puromycin, quinacrine, radicicol, radioactive isotopes, raltitrexed, rapamycin, rasburicase, salinosporamide A, sargramostim, sunitinib, temozolomide, teniposide, tetracaine, 6-thioguanine, thiotepa, topotecan, toremifene, trastuzumab, treosulfan, tretinoin, valrubicin, vinblastine, vincristine, vindesine, vinorelbine, zoledronate, or a combination thereof.

The antimetabolite may be, e.g., methotrexate, 6-mercaptopurine, 6-thioguanine, cytarabine, 5-fluorouracil decarbazine, cladribine, pemetrexed, gemcitabine, capecitabine, hydroxyurea, mercaptopurine, fludarabine, pralatrexate, clofarabine, cytarabine, decitabine, floxuridine, nelarabine, trimetrexate, thioguanine, pentostatin, or a combination thereof.

The alkylating agent may be, e.g., mechlorethamine, thiotepa, chlorambucil, melphalan, carmustine (BSNU), lomustine (CCNU), cyclothosphamide, busulfan, dibromomannitol, streptozotocin, mitomycin C, cis-dichlorodiamine platinum (II) (DDP) cisplatin, altretamine, cyclophosphamide, ifosfamide, hexamethylmelamine, altretamine, procarbazine, dacarbazine, temozolomide, streptozocin, carboplatin, cisplatin, oxaliplatin, uramustine, bendamustine, trabectedin, semustine, or a combination thereof.

The anthracycline may be, e.g., daunorubicin, doxorubicin, aclarubicin, aldoxorubicin, amrubicin, annamycin, carubicin, epirubicin, Idarubicin, mitoxantrone, valrubicin, or a combination thereof.

The antibiotic may be, e.g., dactinomycin, bleomycin, mithramycin, anthramycin (AMC), ampicillin, bacampicillin, carbenicillin, cloxacillin, dicloxacillin, flucloxacillin, mezlocillin, nafcillin, oxacillin, piperacillin, pivampicillin, pivmecillinam, ticarcillin, aztreonam, imipenem, doripenem, ertapenem, meropenem, cephalosporins, clarithromycin, dirithromycin, roxithromycin, telithromycin, lincomycin, pristinamycin, quinupristin, amikacin, gentamicin, kanamycin, neomycin, netilmicin, paromomycin, tobramycin, streptomycin, sulfamethizole, sulfamethoxazole, sulfisoxazole, demeclocycline, minocycline, oxytetracycline, tetracycline, penicillin, amoxicillin, cephalexin, erythromycin, clarithromycin, azithromycin, ciprofloxacin, levofloxacin, ofloxacin, doxycycline, clindamycin, metronidazole, tigecycline, chloramphenicol, metronidazole, tinidazole, nitrofurantoin, vancomycin, teicoplanin, telavancin, linezolid, cycloserine, rifamycins, polymyxin B, bacitracin, viomycin, capreomycin, quinolones, daunorubicin, doxorubicin, 4′-deoxydoxorubicin, epirubicin, idarubicin, plicamycin, mitomycin-c, mitoxantrone, or a combination thereof.

The anti-mitotic agent may be, e.g., vincristine, vinblastine, vinorelbine, docetaxel, estramustine, ixabepilone, paclitaxel, maytansinoid, a dolastatin, a cryptophycin, or a combination thereof.

The signal transduction inhibitor may be, e.g., imatinib, trastuzumab, erlotinib, sorafenib, sunitinib, temsirolimus, vemurafenib, lapatinib, bortezomib, cetuximab panitumumab, matuzumab, gefitinib, STI 571, rapamycin, flavopiridol, imatinib mesylate, vatalanib, semaxinib, motesanib, axitinib, afatinib, bosutinib, crizotinib, cabozantinib, dasatinib, entrectinib, pazopanib, lapatinib, vandetanib, or a combination thereof.

The gene expression modulator may be, e.g., a siRNA, a shRNA, an antisense oligonucleotide, an HDAC inhibitor, or a combination thereof. An HDAC inhibitor may be, e.g., trichostatin A, trapoxin B, valproic acid, vorinostat, belinostat, LAQ824, panobinostat, entinostat, tacedinaline, mocetionstat, givinostat, resminostat, abexinostat, quisinostat, rocilinostat, practinostat, CHR-3996, butyric acid, phenylbutyric acid, 4SC202, romidepsin, sirtinol, cambinol, EX-527, nicotinamide, or a combination thereof. An antisense oligonucleotide may be, e.g., custirsen, apatorsen, AZD9150, trabadersen, EZN-2968, LErafAON-ETU, or a combination thereof. An siRNA may be, e.g., ALN-VSP, CALAA-01, Atu-027, SPC2996, or a combination thereof.

The hormone therapy may be, e.g., a luteinizing hormone-releasing hormone (LHRH) antagonist. The hormone therapy may be, e.g., firmagon, leuproline, goserelin, buserelin, flutamide, bicalutadmide, ketoconazole, aminoglutethimide, prednisone, hydroxyl-progesterone caproate, medroxy-progesterone acetate, megestrol acetate, diethylstil-bestrol, ethinyl estradiol, tamoxifen, testosterone propionate, fluoxymesterone, flutamide, raloxifene, droloxifene, iodoxyfene, 4-hydroxytamoxifen, trioxifene, keoxifene, LY117018, onapristone, toremifine citrate, megestrol acetate, exemestane, fadrozole, vorozole, letrozole, anastrozole, nilutamide, tripterelin, histerelin, arbiraterone, medroxyprogesterone acetate, diethylstilbestrol, premarin, fluoxymesterone, tretinoin, fenretinide, troxacitabine, or a combination thereof.

The apoptosis inducers may be, e.g., a recombinant human TNF-related apoptosis-inducing ligand (TRAIL), camptothecin, bortezomib, etoposide, tamoxifen, or a combination thereof.

The angiogenesis inhibitors may be, e.g., sorafenib, sunitinib, pazopanib, everolimus or a combination thereof.

The immunotherapy agent may be, e.g., a monoclonal antibody, cancer vaccine (e.g., a dendritic cell (DC) vaccine), oncolytic virus, cytokine, adoptive T cell therapy, Bacille Calmette-Guérin (BCG), GM-CSF, thalidomide, lenalidomide, pomalidomide, imiquimod, or a combination thereof. The monoclonal antibody may be, e.g., anti-CTLA4, anti-PD1, anti-PD-L1, anti-LAG3, anti-KIR, or a combination thereof. The monoclonal antibody may be, e.g., alemtuzumab, trastuzumab, ibritumomab tiuxetan, brentuximab vedotin, trastuzumab, ado-trastuzumab emtansine, blinatumomab, bevacizumab, cetuximab, pertuzumab, panitumumab, ramucirumab, obinutuzumab, ofatumumab, rituximab, pertuzumab, tositumomab, gemtuzumab ozogamicin, tositumomab, or a combination thereof. The cancer vaccine may be, e.g., Sipuleucel-T, BioVaxID, NeuVax, DCVax, SuVaxM, CIMAvax®, Provenge,®, hsp110 chaperone complex vaccine, CDX-1401, MIS416, CDX-110, GVAX Pancreas, HyperAcute™ Pancreas, GTOP-99 (MyVax®), or Imprime PGG®. The oncolytic virus may be, e.g., talimogene laherparepvec. The cytokine may be, e.g., IL-2, IFNα, or a combination thereof. The adoptive T cell therapy may be, e.g., tisagenlecleucel, axicabtagene ciloleucel, or a combination thereof.

The DNA damage repair inhibitor may be, e.g., a PARP inhibitor, a DNA-PK Inhibitor, a cell checkpoint kinase inhibitor, or a combination thereof. The PARP inhibitor may be, e.g., olaparib, rucaparib, veliparib (ABT-888), niraparib (ZL-2306), iniparib (BSI-201), talazoparib (BMN 673), 2X-121, CEP-9722, KU-0059436 (AZD2281), PF-01367338, AZD5305, AZD9574, seneparib (IMP4297), fluzoparib (SHR-3162), XIN005104, NMS-293 or a combination thereof. The DNA-PK inhibitor may be AZD7648, nedisertib (M3814), M9831, or BAY-8400. The cell checkpoint kinase inhibitor may be, e.g., RP-6306, MK-1775 or AZD1775, AZD7762, LY2606368, PF-0477736, AZD0156, GDC-0575, ARRY-575, CCT245737, PNT-737 or a combination thereof.

The ionizing radiation therapy and radioligand therapy approaches are known in the art. One or more of these approaches may be combined with the methods described herein.

### EXAMPLES

The following examples were meant to illustrate the invention. They were not meant to limit the invention in any way.

Reactions were typically performed at room temperature (rt) under a nitrogen (N₂) atmosphere using dry solvents (Sure/Seal™) if not described otherwise in the Intermediates and Compounds below. Reactions were monitored by TLC or by injection of a small aliquot on a Waters Acquity-H UPLC® Class system using an Acquity® UPLC HSS C18 2.1×30 mm column eluting with a gradient (1.86 min) of acetonitrile (15% to 98%) in water (both containing 0.1% formic acid). Purifications by preparative HPLC were performed on a Teledyne Isco Combi Flash® EZ Prep system using either Phenomenex Gemini® 5 μm NX-C18 110 Å 150×21.2 mm column at a flow of 40 mL/min over 12 min (<100 mg or multiple injections of <100 mg) or HP C18 RediSed® Rf gold column (>100 mg) eluting with an appropriate gradient of acetonitrile in water (both containing 0.1% formic acid) unless otherwise specified. The gradient was selected based on the retention time observed by reaction monitoring on the Waters Acquity-H UPLCs Class system (see above). Fractions containing the desired compounds were combined and finally lyophilized. Purifications by silica gel chromatography were performed on a Teledyne Isco Combi Flash® Rf system using RediSep® Rf silica gel columns of appropriate sizes. Purity of final Compounds was assessed by injection of a small aliquot on a Waters Acquity-H UPLC® Class system using an Acquity® UPLC BEH C18 2.1×50 mm column eluting with a gradient (7 min) of acetonitrile (2% to 98%) in water (both containing 0.1% formic acid).

### Intermediates

The following intermediates were used to prepare exemplary compounds of the invention described below.

TABLE 3

| | |
|---|---|
| 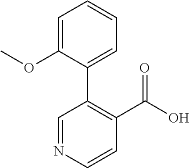 [SMILES: COc1ccccc1-c1cnccc1C(=O)O] | Intermediate 1 |
| 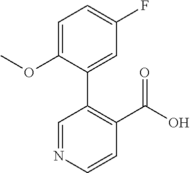 [SMILES: COc1ccc(F)cc1-c1cnccc1C(=O)O] | Intermediate 2 |
| 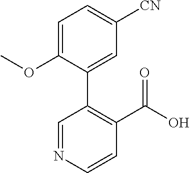 [SMILES: COc1ccc(C#N)cc1-c1cnccc1C(=O)O] | Intermediate 3 |
| 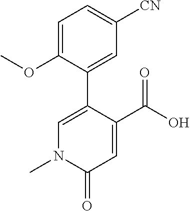 [SMILES: COc1ccc(C#N)cc1-c1cn(C)c(=O)cc1C(=O)O] | Intermediate 4 |
| 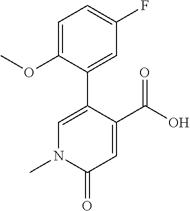 [SMILES: COc1ccc(F)cc1-c1cn(C)c(=O)cc1C(=O)O] | Intermediate 5 |
| 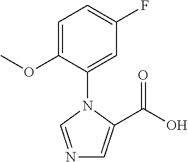 [SMILES: COc1ccc(F)cc1-n1cncc1C(=O)O] | Intermediate 6 |
| 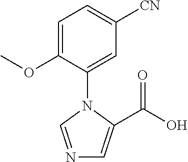 [SMILES: COc1ccc(C#N)cc1-n1cncc1C(=O)O] | Intermediate 7 |
| 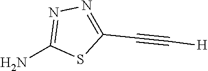 | Intermediate 8 |
| 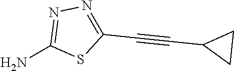 [SMILES: Nc1nnc(C#CC2CC2)s1] | Intermediate 9 |
| 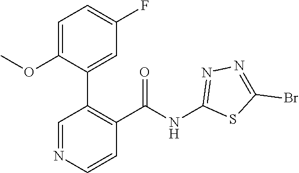 [SMILES: COc1ccc(F)cc1-c1cnccc1C(=O)Nc1nnc(Br)s1] | Intermediate 10 |
| 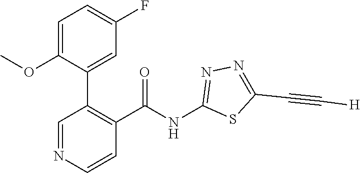 [SMILES: C#Cc1nnc(NC(=O)c2ccncc2-c2cc(F)ccc2OC)s1] | Intermediate 11 |
| 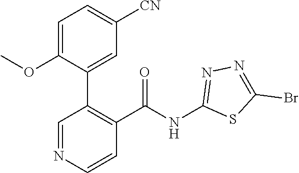 [SMILES: COc1ccc(C#N)cc1-c1cnccc1C(=O)Nc1nnc(Br)s1] | Intermediate 12 |
| 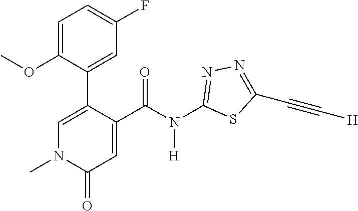 [SMILES: C#Cc1nnc(NC(=O)c2cc(=O)n(C)cc2-c2cc(F)ccc2OC)s1] | Intermediate 13 |
| 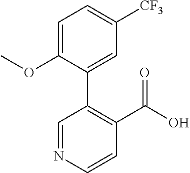 [SMILES: COc1ccc(C(F)(F)F)cc1-c1cnccc1C(=O)O] | Intermediate 14 |
| 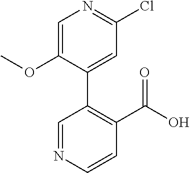 [SMILES: COc1cnc(Cl)cc1-c1cnccc1C(=O)O] | Intermediate 15 |
| 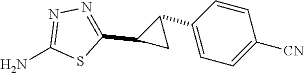 | Intermediate 16 |
| 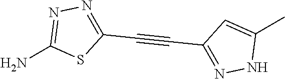 | Intermediate 17 |
| 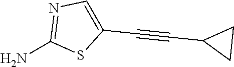 [SMILES: Nc1ncc(C#CC2CC2)s1] | Intermediate 18 |
| 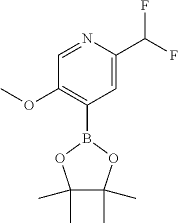 [SMILES: COc1cnc(C(F)F)cc1B1OC(C)(C)C(C)(C)O1] | Intermediate 19 |
| 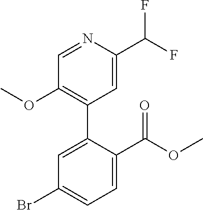 | Intermediate 20 |
| 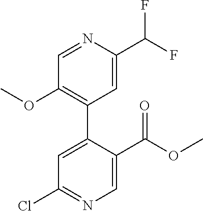 | Intermediate 21 |
| 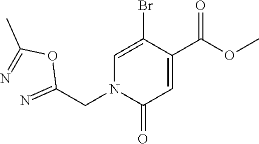 [SMILES: COC(=O)c1cc(=O)n(CC(=N)OC(C)=N)cc1Br] | Intermediate 22 |
| 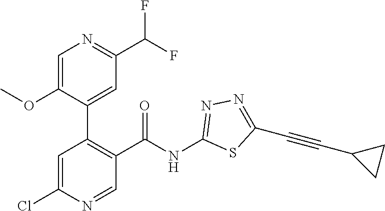 [SMILES: COc1cnc(C(F)F)cc1-c1cc(Cl)ncc1C(=O)Nc1nnc(C#CC2CC2)s1] | Intermediate 23 |
| 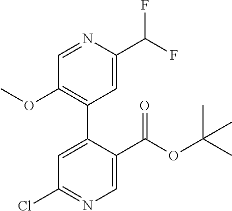 [SMILES: COc1cnc(C(F)F)cc1-c1cc(Cl)ncc1C(=O)OC(C)(C)C] | Intermediate 24 |
| 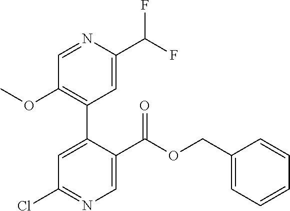 [SMILES: COc1cnc(C(F)F)cc1-c1cc(Cl)ncc1C(=O)OCc1ccccc1] | Intermediate 25 |
| 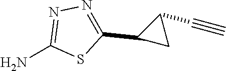 | Intermediate 26 |
| 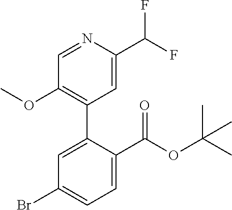 [SMILES: COc1cnc(C(F)F)cc1-c1cc(Br)ccc1C(=O)OC(C)(C)C] | Intermediate 27 |
| 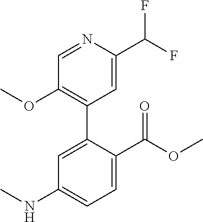 | Intermediate 28 |
| 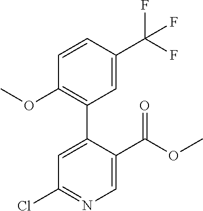 | Intermediate 29 |
| 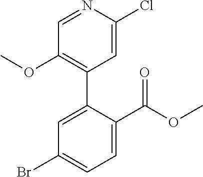 | Intermediate 30 |
| 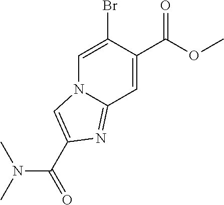 | Intermediate 31 |
| 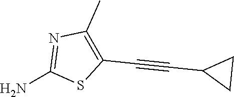 | Intermediate 32 |
| 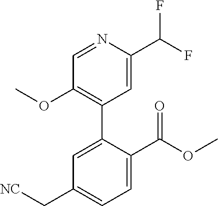 | Intermediate 33 |
| 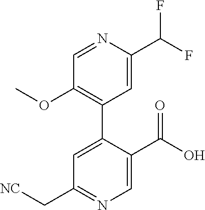 [SMILES: COc1cnc(C(F)F)cc1-c1cc(CC#N)ncc1C(=O)O] | Intermediate 34 |
| 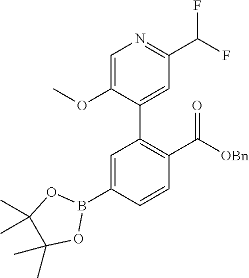 [SMILES: COc1cnc(C(F)F)cc1-c1cc(B2OC(C)(C)C(C)(C)O2)ccc1C(=O)OCc1ccccc1] | Intermediate 35 |

### Intermediate 1/3-(2-methoxyphenyl) isonicotinic acid

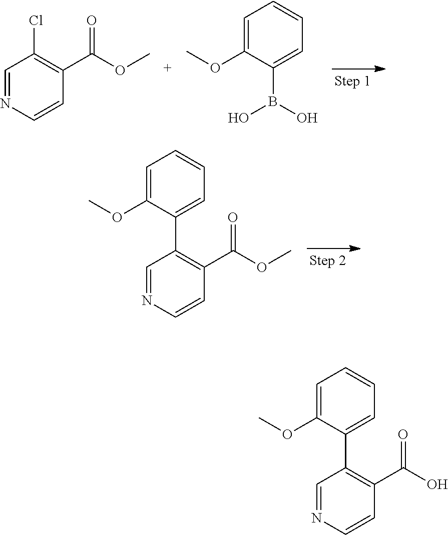

### Step 1/methyl 3-(2-methoxyphenyl)isonicotinate

N₂ was bubbled for 30 min in a mixture of methyl 3-chloropyridine-4-carboxylate (10.00 g, 58.28 mmol) and (2-methoxyphenyl)boronic acid (11.50 g, 75.68 mmol) in H₂0 (50 mL) and 1-4-dioxanne (125 mL). To the resulting mixture was added Pd(OAc)₂ (655 mg, 2.92 mmol) and dicyclohexyl-[2-(2,6-dimethoxyphenyl)phenyl]phosphane (2.39 g, 5.83 mmol). N₂ was bubbled for an extra 15 min, and the resulting mixture was stirred at 90 °C for 4 hrs. The reaction mixture was cooled to rt, poured in water, and extracted with EtOAc (3×). The combined organic layers were washed with brine, dried over Na₂SO₄, filtered, and concentrated in vacuo. The residue was purified by silica gel chromatography eluting with a gradient of EtOAc (10 to 60%) in Heptane. Appropriate fractions were combined and concentrated in vacuo to afford methyl 3-(2-methoxyphenyl)pyridine-4-carboxylate (12.7 g, 90% yield) as a light yellow solid.

### Step 2/Intermediate 1

To a solution of methyl 3-(2-methoxyphenyl)pyridine-4-carboxylate (12.7 g, 52.2 mmol) in 1-4 dioxane (100 mL) and MeOH (50 mL) was added LiOH·H₂O (1 M, 78 mL, 78 mmol). The resulting solution was stirred at 60 °C for 6 hrs. The volatiles were evaporated in vacuo, the resulting solution was diluted with 100 mL of water then formic acid (4.0 mL, 106 mmol) was added dropwise. The resulting white solid was filtered, washed with water twice and dried in vacuo to afford Intermediate 1 (11.7 g, 98% yield) as a white solid. LCMS m/z 230.1 [M+H]⁺.

### Intermediate 2/3-(5-fluoro-2-methoxyphenyl)isonicotinic acid

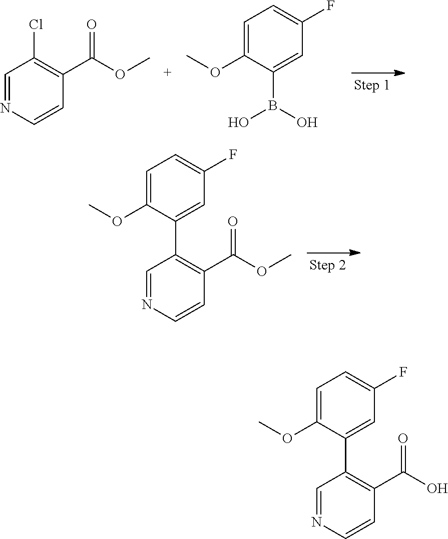

### Step 1/methyl 3-(5-fluoro-2-methoxyphenyl)isonicotinate

N₂ was bubbled through a biphasic mixture of methyl 3-chloropyridine-4-carboxylate (2.20 g, 12.82 mmol), (5-fluoro-2-methoxy-phenyl)boronic acid (3.05 g, 17.95 mmol), dicyclohexyl-[2-(2,6-dimethoxyphenyl)phenyl]phosphane (611 mg, 1.49 mmol), K₂CO₃ (5.28 g, 38.2 mmol) in water (6 mL) and 1,4-dioxane (20 mL) while sonicating for 15 min. Pd(OAc)₂ (183 mg, 814 μmol) was then added and the tube sealed. The reaction mixture was stirred at 90 °C overnight. The reaction mixture was cooled to rt, poured in water, and extracted with EtOAc (3×). The combined organic layers were washed with brine, dried over Na₂SO₄, filtered, and concentrated in vacuo. The residue (silica dry pack) was purified by silica gel chromatography eluting with a gradient of EtOAc (5 to 70%) in heptane. Appropriate fractions were combined and concentrated in vacuo to afford methyl 3-(5-fluoro-2-methoxy-phenyl)pyridine-4-carboxylate (3.26 g, 97% yield) as light yellow solid.

### Step 2/Intermediate 2

To a solution of methyl 3-(5-fluoro-2-methoxy-phenyl)pyridine-4-carboxylate (3.26 g, 12.5 mmol) in water (8.0 mL), MeOH (8.0 mL) and 1,4-Dioxane (32 mL) was added LiOH·H₂O, 98% (848 mg, 20.2 mmol) in one portion. The reaction mixture was stirred at 80° for 2 hrs. The reaction mixture was cooled to 0 °C and formic acid, 97% (1.79 mL, 47.4 mmol) was added. After stirring for 5 min, the resulting precipitate was filtered, washed with water (3×) and dried in vacuo to afford Intermediate 2 (2.57 g, 83% yield). LCMS m/z 248.1 [M+H]+.

### Intermediate 3/3-(5-cyano-2-methoxyphenyl)isonicotinic acid

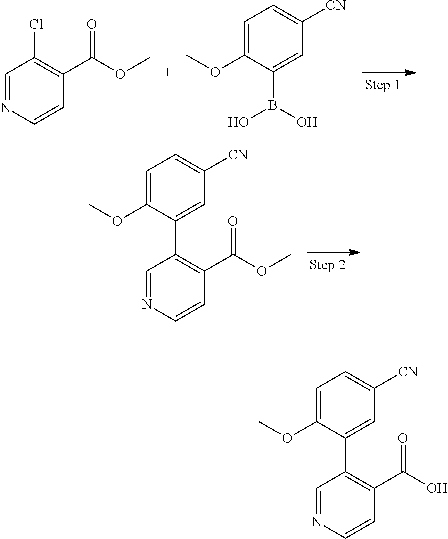

### Step 1/methyl 3-(5-cyano-2-methoxyphenyl)isonicotinate

N₂ was bubbled through a biphasic mixture of methyl 3-chloropyridine-4-carboxylate (300 mg, 1.75 mmol), (5-cyano-2-methoxy-phenyl)boronic acid (464 mg, 2.62 mmol), dicyclohexyl-[2-(2,6-dimethoxyphenyl)phenyl]phosphane (108 mg, 262 μmol), K₂CO₃ (728 mg, 5.27 mmol) in 1,4-dioxane (3 mL)/H₂O (1 mL) while sonicating for 15 min. Pd(OAc)₂ (41.2 mg, 183 μmol) was then added and the tube sealed. The reaction mixture was stirred at 90 °C overnight. The reaction mixture was cooled to rt, poured in water, and extracted with EtOAc (3×). The combined organic layers were washed with brine, dried over Na₂SO₄, filtered, and concentrated in vacuo. The residue was purified by silica gel chromatography eluting with a gradient of EtOAc (5 to 100%) in Heptane. Appropriate fraction were combined and concentrated in vacuo to afford methyl 3-(5-cyano-2-methoxy-phenyl)pyridine-4-carboxylate (397 mg, 85% yield).

### Step 2/Intermediate 3

To a solution of methyl 3-(5-cyano-2-methoxy-phenyl)pyridine-4-carboxylate (397 mg, 1.48 mmol) in 1,4-Dioxane (4 mL), MeOH (1.0 mL) and H₂O (1.0 mL) was added LiOH·H₂O (101 mg, 2.40 mmol) in one portion. The reaction mixture was stirred at 80 °C for 2 hrs. The reaction mixture was cooled to 0 °C and formic acid, 97% (200 μL, 5.30 mmol) was added. After 5 min, the resulting precipitate was filtered, washed with water (3×) and dried in vacuo to afford Intermediate 3 (310 mg, 83% yield). LCMS m/z 255.1 [M+H]⁺.

### Intermediate 4/5-(5-cyano-2-methoxyphenyl)-1-methyl-2-oxo-1,2-dihydropyridine-4-carboxylic acid

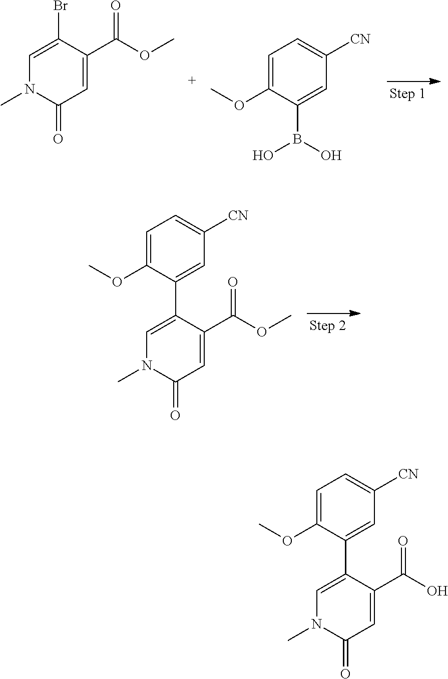

### Step 1/methyl 5-(5-cyano-2-methoxyphenyl)-1-methyl-2-oxo-1,2-dihydropyridine-4-carboxylate

N₂ was bubbled through a biphasic mixture of methyl 5-bromo-1-methyl-2-oxo-pyridine-4-carboxylate (2.70 g, 11.0 mmol), (5-cyano-2-methoxy-phenyl)boronic acid (2.33 g, 13.2 mmol), dicyclohexyl-[2-(2,6-dimethoxyphenyl)phenyl]phosphane (674 mg, 1.64 mmol), K₂CO₃ (4.57 g, 33.1 mmol) in 1,4-dioxane (25 mL)/H₂O (8.5 mL) while sonicating for 15 min. Pd(OAc)₂ (185 mg, 823 μmol) was then added and the tube sealed. The reaction mixture was stirred at 90 °C overnight. The reaction mixture was cooled to rt, poured in water and extracted with EtOAc (3×). The combined organic layers were washed with brine, dried over Na₂SO₄, filtered, and concentrated in vacuo. The residue was purified by silica gel chromatography eluting with a gradient of EtOAc (5 to 100%) in Heptane. Appropriate fractions were combined and concentrated in vacuo to afford methyl 5-(5-cyano-2-methoxy-phenyl)-1-methyl-2-oxo-pyridine-4-carboxylate (1.1 g, 34% yield).

### Step 2/Intermediate 4

To a suspension of methyl 5-(5-cyano-2-methoxy-phenyl)-1-methyl-2-oxo-pyridine-4-carboxylate (1.55 g, 5.20 mmol) and LiOH·H₂O (364 mg, 8.68 mmol) in 1,4-Dioxane (30 mL) was added MeOH (10 mL) and H₂O (10 mL). The reaction mixture was stirred at 40 °C for 2 hrs. The resulting mixture was cooled to 0 °C and subsequently formic acid (784 μL, 20.8 mmol) was added. The resulting precipitate was filtered, washed with water, and dried in vacuo to afford Intermediate 4 (563 mg, 38% yield). LCMS m/z 285.1 [M+H]⁺.

### Intermediate 5/5-(5-fluoro-2-methoxyphenyl)-1-methyl-2-oxo-1,2-dihydropyridine-4-carboxylic acid

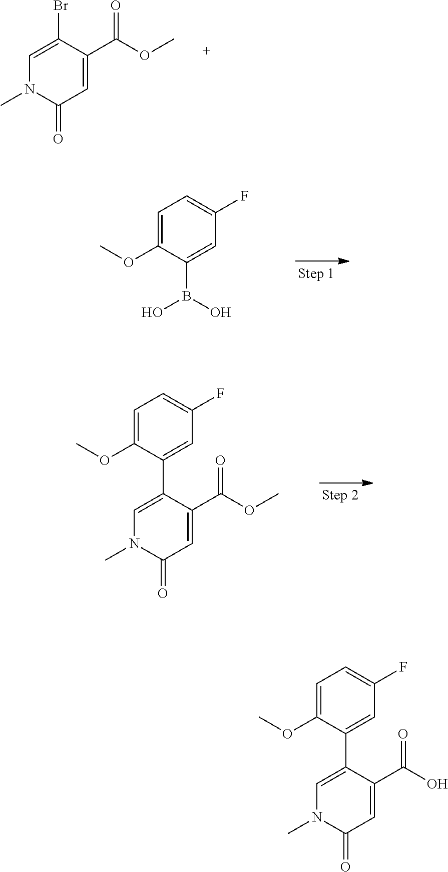

### Step 1/methyl 5-(5-fluoro-2-methoxyphenyl)-1-methyl-2-oxo-1,2-dihydropyridine-4-carboxylate

N₂ was bubbled for 30 min in a mixture of methyl 5-bromo-1-methyl-2-oxo-pyridine-4-carboxylate (921 mg, 3.74 mmol) in H₂O (5 mL) and 1-4-dioxanne (25 mL). To the resulting mixture was added K₂CO₃ (1.75 g, 12.66 mmol) and [2-(2-aminophenyl)phenyl]palladium dicyclohexyl-[2-(2,6-dimethoxyphenyl)phenyl]phosphane;methanesulfonate (164 mg, 187 μmol). N₂ was bubbled for an extra 15 min, and the resulting mixture was stirred at 90 °C for 4 hrs. The reaction mixture was cooled to rt, poured in water, and extracted with EtOAc (3×). The combined organic layers were washed with brine, dried over Na₂SO₄, filtered, and concentrated in vacuo. The residue was purified by silica gel chromatography eluting with a gradient of EtOAc (10 to 100%) in Heptane. Appropriate fractions were combined and concentrated in vacuo afford methyl 5-(5-fluoro-2-methoxy-phenyl)-1-methyl-2-oxo-pyridine-4-carboxylate (762 mg, 70% yield) as a light beige solid.

### Step 2/Intermediate 5

To a solution of methyl 5-(5-fluoro-2-methoxy-phenyl)-1-methyl-2-oxo-pyridine-4-carboxylate (762 mg, 2.62 mmol) in water (4 mL) and 1,4-dioxane (5 mL) was added LiOH·H₂O (2 M, 3.90 mL, 3.90 mmol). The resulting solution was stirred at 50 °C for 1 hour. The volatiles were evaporated in vacuo, the resulting residue was diluted with 100 mL of water then formic acid (350 μL, 9.28 mmol) was added dropwise. The resulting white precipitate was filtered, washed with water (2×) and dried in vacuo to afford Intermediate 5 (722 mg, 99% yield) as a white solid. LCMS m/z 278.1 [M+H]⁺.

### Intermediate 6/1-(5-fluoro-2-methoxyphenyl)-1H-imidazole-5-carboxylic acid

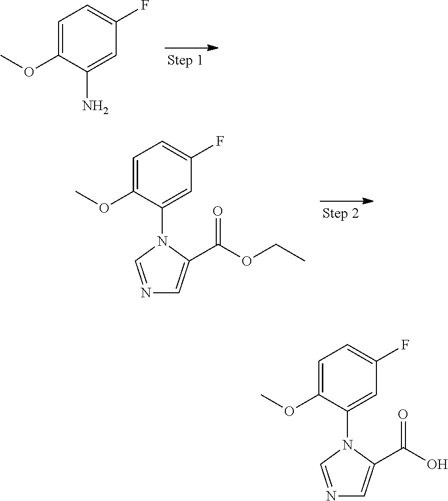

### Step 1/ethyl 1-(5-fluoro-2-methoxyphenyl)-1H-imidazole-5-carboxylate

In 30 mL seal tube, 5-fluoro-2-methoxyaniline (400 mg, 2.83 mmol) was dissolved in EtOH (5 mL) at rt, followed by the addition of ethyl 2-oxoacetate (290 mg, 2.84 mmol). The reaction mixture was stirred for 1 hour at rt. To the resulting mixture was added K₂CO₃ (783 mg, 5.66 mmol) and p-toluenesulfonylmethyl isocyanide (663 mg, 3.40 mmol). The reaction mixture was heated to 80 °C for 3 hrs. The reaction mixture was cooled to rt, quenched in water (50 mL), and extracted with EtOAc (3×). The combined organic layers were dried over Na₂SO₄, filtered, and concentrated in vacuo. The residue was purified by silica gel chromatography eluting with of EtOAc (60%) in hexanes. Appropriate fractions were combined and concentrated in vacuo to afford ethyl 1-(5-fluoro-2-methoxyphenyl)-1H-imidazole-5-carboxylate (200 mg, 27% yield).

### Step 2/Intermediate 6

In 10 mL sealed tube, ethyl 1-(5-fluoro-2-methoxyphenyl)-1H-imidazole-5-carboxylate (200 mg, 0.757 mmol) was dissolved in THF:H₂O (1:1, 1 mL) at rt, followed by the addition of LiOH·H₂O (90 mg, 2.27 mmol). The reaction mixture was stirred at rt for 4 hrs. The reaction mixture was concentrated under vacuum. The resulting crude was diluted with water and acidified with HCl (1N) to afford after lyophilization 1-(5-fluoro-2-methoxyphenyl)-1H-imidazole-5-carboxylic acid (110 mg, 62%) as sticky solid material which was used without further purification. LCMS m/z 236.8 [M+H]⁺.

### Intermediate 7/1-(5-cyano-2-methoxyphenyl)-1H-imidazole-5-carboxylic acid

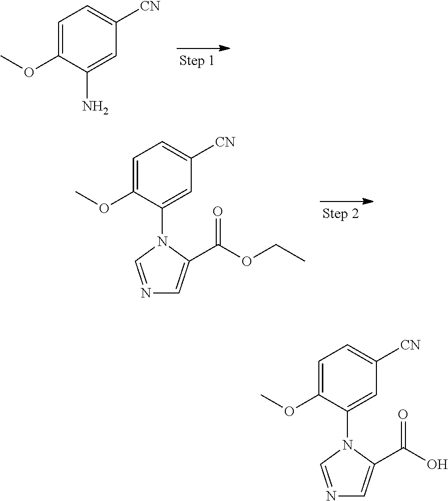 [SMILES: COc1ccc(C#N)cc1N ; CCOC(=O)c1cncn1-c1cc(C#N)ccc1OC ; COc1ccc(C#N)cc1-n1cncc1C(=O)O]

### Step 1/ethyl 1-(5-cyano-2-methoxyphenyl)-1H-imidazole-5-carboxylate

In 30 mL seal tube, 3-amino-4-methoxybenzonitrile (350 mg, 2.36 mmol) was dissolved in ethanol (5 mL) at rt, followed by the addition of ethyl 2-oxoacetate (289 mg, 2.83 mmol) and the mixture was stirred for 1 hour. To the resulting reaction mixture was added p-toluenesulfonylmethyl isocyanide (553 mg, 2.83 mmol) and K₂CO₃ (652 mg, 4.72 mmol), then the reaction mixture was heated to 80 °C for 3 hrs.

The resulting mixture was cooled to rt, quenched in water (50 mL) and extracted with EtOAc (3×). The combined organic layers were dried over Na₂SO₄, filtered, and concentrated in vacuo. The residue was purified by silica gel chromatography eluting EtOAc (65%) in hexanes. The appropriate fractions were combined and concentrated to afford ethyl 1-(5-cyano-2-methoxyphenyl)-1H-imidazole-5-carboxylate (150 mg, 23% yield).

### Step 2/Intermediate 7

In 10 mL round bottom flask, ethyl 1-(5-cyano-2-methoxyphenyl)-1H-imidazole-5-carboxylate (150 mg, 0.553 mmol) was dissolved in THF:H₂O (1:1, 2 mL) at rt, followed by the addition of LiOH·H₂O (70 mg, 1.7 mmol). The reaction mixture was stirred at rt for 16 hrs. The reaction mixture was concentrated in vacuo, diluted with water (1 mL), and acidified with 1N HCl at 0 °C. The aqueous layer was extracted with MeOH:DCM (1:9) (3×5 mL). The combined organics layers were dried over Na₂SO₄, filtered, and concentrated in vacuo to afford Intermediate 7 (120 mg, 83%) which was used without any further purification. LCMS m/z 244.0 [M+H]⁺.

### Intermediate 8/5-ethynyl-1,3,4-thiadiazol-2-amine

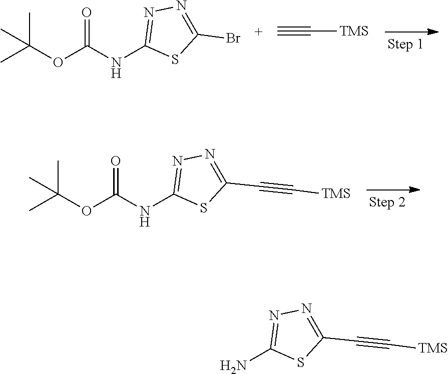

### Step 1/tert-butyl (5-(cyclopropylethynyl)-1,3,4-thiadiazol-2-yl)carbamate

N₂ was bubbled through a solution of tert-butyl N-(5-bromo-1,3,4-thiadiazol-2-yl)carbamate (15.0 g, 53.5 mmol), ethynyl(trimethyl)silane (60.5 mL, 428 mmol) NEt₃ (60 mL, 431 mmol) in dry DMF (60.0 mL) while sonicating for 15 min. Pd(PPh₃)₄ (6.19 g, 5.35 mmol) was added and the resulting reaction mixture was stirred at 50 °C for 48 hrs. The yellow suspension was cooled to rt, filtered and the solids washed with Et₃N (×1). The filtrate was concentrated in vacuo and the resulting DMF solution used as such in the next step.

### Step 2/Intermediate 8

To a solution of tert-butyl (5-(cyclopropylethynyl)-1,3,4-thiadiazol-2-yl)carbamate (1.08 g, 4.08 mmol) in dry DCM (40 mL) was added TFA (6.4 mL, 83 mmol). The reaction mixture was stirred at rt for 2 hrs. Volatiles were removed in vacuo and EtOAc and NaOH (2N, 3.0 mL, 6.0 mmol) were added. The aqueous phases were extracted with EtOAc (5×). The combined organic phases were dried over MgSO4, filtered, and evaporated to dryness. The residue (silica dry pack) was purified by silica gel chromatography eluting with a gradient of MeOH (0 to 10%) in DCM. Appropriate fractions were combined and concentrated in vacuo to afford Intermediate 8 (380 mg, 56% yield) as a light orange solid. LCMS m/z 126.1 [M+H]⁺.

### Intermediate 9/5-(cyclopropylethynyl)-1,3,4-thiadiazol-2-amine

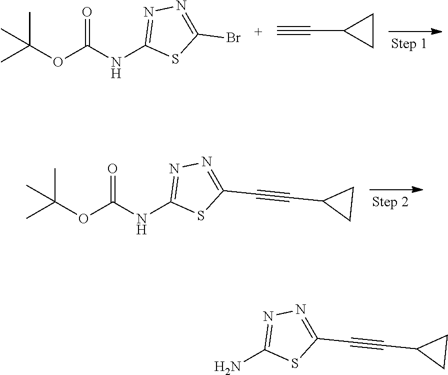

### Step 1/tert-butyl (5-(cyclopropylethynyl)-1,3,4-thiadiazol-2-yl)carbamate

N₂ was bubbled through a solution of tert-butyl N-(5-bromo-1,3,4-thiadiazol-2-yl)carbamate (1.20 g, 4.28 mmol), ethynylcyclopropane (2.90 mL, 34.3 mmol), NEt₃ (4.80 mL, 34.4 mmol) in dry DMF (4.0 mL) while sonicating for 15 min. Pd(PPh₃)₄ (495 mg, 428 μmol) was added and the reaction mixture was stirred at 50 °C for 48 hrs. The resulting yellow suspension was cooled to rt, filtered and the solid washed with Et₃N (1×). The filtrate was concentrated in vacuo, diluted with EtOAc, washed with brine, dried over MgSO4, filtered and concentrated. The residue (silica dry pack) was purified by silica gel chromatography eluting with a gradient of EtOAc (5 to 70%) in Heptane. Appropriate fractions were combined and concentrated in vacuo to afford tert-butyl (5-(cyclopropylethynyl)-1,3,4-thiadiazol-2-yl)carbamate (1.08 g, 95% yield).

### Step 2/Intermediate 9

To a solution of tert-butyl (5-(cyclopropylethynyl)-1,3,4-thiadiazol-2-yl)carbamate (1.08 g, 4.08 mmol) in dry DCM (40 mL) was added TFA (6.4 mL, 83 mmol). The reaction mixture was stirred at rt for 2 hrs. Volatiles were removed in vacuo and EtOAc and NaOH (2N, 3.0 mL, 6.0 mmol) were added. The aqueous phase was extracted with EtOAc (5×). The combined organic phases were dried over MgSO₄, filtered, and evaporated to dryness. The residue (silica dry pack) was purified by silica gel chromatography eluting with a gradient of MeOH (0 to 10%) in DCM. Appropriate fractions were combined and concentrated in vacuo to afford Intermediate 9 (380 mg, 56% yield) as a light orange solid. LCMS m/z 166.0 [M+H]⁺.

### Intermediate 10/N-(5-bromo-1,3,4-thiadiazol-2-yl)-3-(5-fluoro-2-methoxyphenyl)isonicotinamide

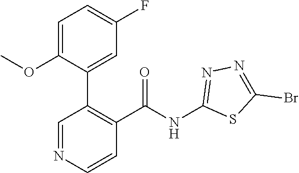 [SMILES: COc1ccc(F)cc1-c1cnccc1C(=O)Nc1nnc(Br)s1]

In a 100 mL 3 necked round bottom flask under inert atmosphere of N₂ gas, Intermediate 2 (2.00 g, 8.09 mmol) and 5-bromo-1,3,4-thiadiazol-2-amine (2.18 g, 12.13 mmol) were dissolved in DMF (20 mL). To the resulting solution ay 0 °C was added HOBt (1.63 g, 12.1 mmol) followed by the addition of EDC·HCl (2.32 g, 12.1 mmol). The final reaction mixture was stirred at rt for 16 hrs. The reaction mixture was poured into chilled water and the resulting precipitate was filtered and dried in vacuo. The crude product was purified by silica gel chromatography eluting with MeOH (5%) in DCM. Appropriate fractions were combined and concentrated in vacuo to afford Intermediate 10 (0.80 g, 24% yield). LCMS: m/z 408.8 [M+H]⁺

### Intermediate 11/N-(5-ethynyl-1,3,4-thiadiazol-2-yl)-3-(5-fluoro-2-methoxyphenyl)isonicotinamide

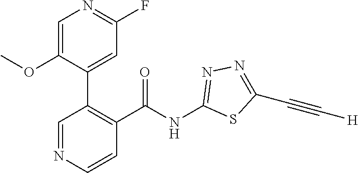 [SMILES: C#Cc1nnc(NC(=O)c2ccncc2-c2cc(F)ncc2OC)s1]

In 25 mL three neck round bottom flask, Intermediate 8 (550 mg, 4.40 mmol) was dissolved in pyridine (5.5 mL) at 0 °C, followed by the addition of Intermediate 2 (1.30 g, 5.28 mmol) and EDC·HCl (1.60 g, 8.80 mmol). The reaction mixture was stirred at rt for 4 hrs. The reaction mixture was quenched with water and extracted with EtOAc (3×). The combined organic layers were further washed with 10% aq. citric acid solution. The organic layer was dried over Na₂SO₄, filtered, and concentrated in vacuo. The residue was purified by silica gel chromatography and the product was eluted using MeOH (3%) in DCM. Appropriate fractions were combined and concentrated in vacuo to afford Intermediate 11 (250 mg, 17%). LCMS: m/z 355.01 [M+H]⁺

### Intermediate 12/N-(5-bromo-1,3,4-thiadiazol-2-yl)-3-(5-cyano-2-methoxyphenyl)isonicotinamide

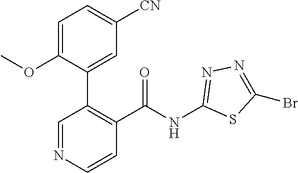 [SMILES: COc1ccc(C#N)cc1-c1cnccc1C(=O)Nc1nnc(Br)s1]

To an ice-cold suspension of 5-bromo-1,3,4-thiadiazol-2-amine (1.75 g, 9.71 mmol) and Intermediate 3 (1.23 g, 4.85 mmol) and HOBt·H₂O (1.11 g, 7.28 mmol) in dry DMF (15 mL) was added EDC (1.40 g, 7.28 mmol). After 5 min at 0 °C, the reaction mixture was stirred at rt overnight then 2 hrs at 50 °C to complete the reaction. The reaction mixture was poured in H₂O and the precipitate was filtered, washed with water. The precipitate (silica dry pack) was purified by silica gel chromatography eluting with a gradient of MeOH (0 to 8%) in DCM. Appropriate fractions were combined and concentrated in vacuo to afford Intermediate 12 (990 mg, 49% yield) as an off-white solid. LCMS: m/z 411.1 [M+H]⁺.

### Intermediate 13/N-(5-ethynyl-1,3,4-thiadiazol-2-yl)-5-(5-fluoro-2-methoxyphenyl)-1-methyl-2-oxo-1,2-dihydropyridine-4-carboxamide

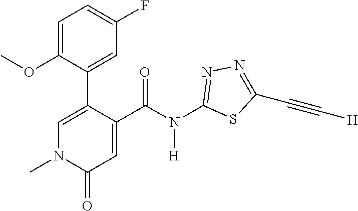 [SMILES: C#Cc1nnc(NC(=O)c2cc(=O)n(C)cc2-c2cc(F)ccc2OC)s1]

In 100 mL single neck flask, Intermediate 5 (2.30 g, 8.30 mmol) was dissolved in pyridine (30 mL) at rt, followed by the addition of Intermediate 8 (1.00 g, 9.96 mmol) and EDC·HCl (7.96 g, 41.5 mmol). The reaction mixture was stirred at rt for 16 hrs. The reaction mixture was quenched in water and extracted with EtOAc (3×). The combined organic layers were dried over Na₂SO₄, filtered, and concentrated in vacuo. The residue was purified by silica gel chromatography eluting with EtOAc (70%) in hexanes. Appropriate fractions were combined and concentrated in vacuo to afford Intermediate 13 (0.840 g, 28% yield). LCMS: m/z 385.2 [M+H].

### Intermediate 14/3-(2-methoxy-5-(trifluoromethyl)phenyl)isonicotinic acid

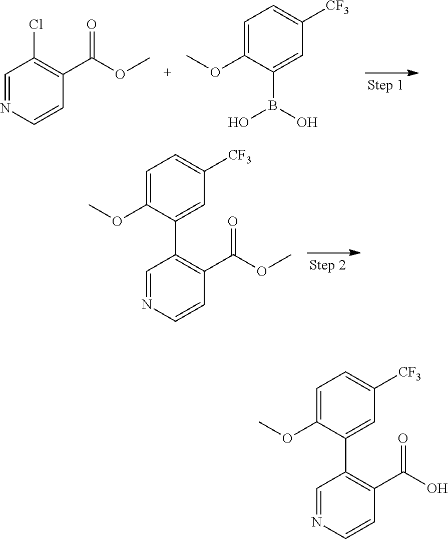

### Step 1/methyl 3-(2-methoxy-5-(trifluoromethyl)phenyl)isonicotinate

N₂ was bubbled for 30 min in a mixture of methyl 3-bromopyridine-4-carboxylate (300 mg, 1.39 mmol) in H₂O (1.0 mL) and 1-4-dioxanne (12 mL). To the resulting mixture was added Pd(OAc)₂ (15.9 mg, 70.8 μmol) and dicyclohexyl-[2-(2,6-dimethoxyphenyl)phenyl]phosphane (57.1 mg, 139 μmol) and K₂CO₃ (575 mg, 4.16 mmol). N₂ was bubbled for an extra 15 min and the resulting mixture was stirred at 90 °C for 4 hrs. The reaction mixture was cooled to rt, poured in water and extracted with EtOAc (3×). The combined organic layers were washed with brine, dried over Na₂SO₄, filtered, and concentrated in vacuo. The residue was purified by silica gel chromatography eluting with a gradient of EtOAc (10 to 60%) in Heptane. Appropriate fractions were combined and concentrated in vacuo to afford methyl 3-[2-methoxy-5-(trifluoromethyl)phenyl]pyridine-4-carboxylate (308 mg, 71% yield) as a white solid.

### Step 2/Intermediate 14

To a solution of methyl 3-[2-methoxy-5-(trifluoromethyl)phenyl]pyridine-4-carboxylate (308 mg, 990 μmol) in 1-4 dioxane (4.0 mL) and MeOH (1.0 mL) and H₂O (1.0 mL) was added LiOH·H₂O (1.49 mL, 1.0 M, 1.49 mmol). The resulting solution was stirred at 50 °C for 1 h. The volatiles were evaporated in vacuo, the resulting solution was diluted with 3 mL of water then formic acid (80 μL, 2.1 mmol) was added dropwise. The resulting white solid was filtered, washed with water twice and dried in vacuo to afford Intermediate 14 (260 mg, 88.4% yield) as a white solid. LCMS m/z 298.1 [M+H]⁺.

### Intermediate 15/2′-chloro-5′-methoxy-[3,4′-bipyridine]-4-carboxylic acid

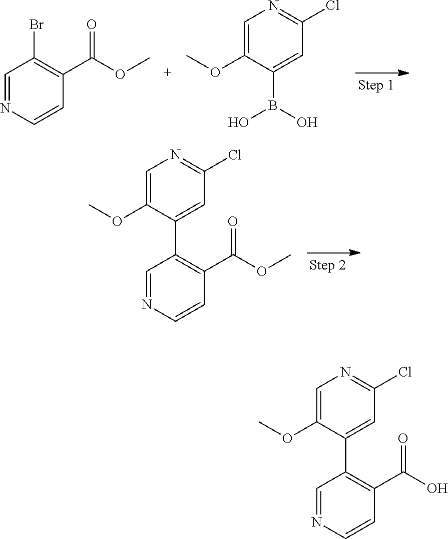

### Step 1/methyl 2′-chloro-5′-methoxy-[3,4′-bipyridine]-4-carboxylate

N₂ was bubbled for 30 min in a mixture of methyl 3-bromopyridine-4-carboxylate (528 mg, 2.44 mmol) and (2-chloro-5-methoxypyridin-4-yl)boronic acid (600 mg, 3.20 mmol) in H₂O (2.0 mL) and 1-4-dioxane (25 mL). To the resulting mixture was added Pd(OAc)₂ (32.6 mg, 145 μmol), dicyclohexyl-[2-(2,6-dimethoxyphenyl)phenyl]phosphane (107 mg, 259 μmol) and K₂CO₃ (1.03 g, 7.45 mmol). N₂ was bubbled for an extra 15 min, and the resulting mixture was stirred at 90 °C for 4 hrs. The reaction mixture was cooled to rt, poured in water, and extracted with EtOAc (3×). The combined organic layers were washed with brine, dried over Na₂SO₄, filtered, and concentrated in vacuo. The residue was purified by silica gel chromatography eluting with a gradient of EtOAc (10 to 80%) in Heptane. Appropriate fractions were combined and concentrated in vacuo to afford methyl 3-(2-chloro-5-methoxy-4-pyridyl)pyridine-4-carboxylate (270 mg, 40% yield) as a white solid.

### Step 2/Intermediate 15

To a solution of methyl 3-(2-chloro-5-methoxy-4-pyridyl)pyridine-4-carboxylate (160 mg, 574 μmol) in 1-4 dioxane (2.0 mL), MeOH (0.5 mL) and H₂O (0.5 mL) was added LiOH·H₂O, 98% (36.3 mg, 865 μmol). The resulting solution was stirred at 50 °C for 1 hour. The volatiles were evaporated in vacuo, the resulting solution was diluted with 3 mL of water then formic acid (50.0 μL, 1.33 mmol) was added dropwise. The resulting white solid was filtered, washed with water twice and dried in vacuo to afford Intermediate 15 (130 mg, 86% yield) as a white solid. LCMS m/z 265.1 [M+H]⁺.

### Intermediate 16/Racemic 4-((1R,2R)-2-(5-amino-1,3,4-thiadiazol-2-yl)cyclopropyl)benzonitrile

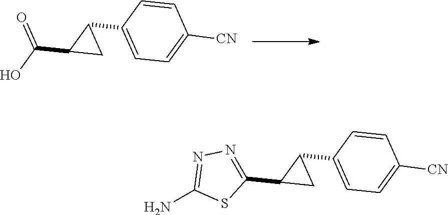

To a mixture of (1R,2R)-2-(4-cyanophenyl)cyclopropane-1-carboxylic acid (395 mg, 1.64 mmol) and thiosemicarbazide, 99% (164.3 mg, 1.80 mmol) was added dropwise POCl₃ (0.6 mL) at 0 °C. The slurry was heated at 90 °C for 4 hrs. Ice water was slowly added to the resin like mixture and vigorously stirred at rt for 1 hour and the pH was adjusted to 9 with NaOH flakes. The slurry was stirred at rt for 1 hour and the resulting precipitate was filtered, washed with water (3×) and dried in vacuo to afford Intermediate 16 (210 mg, 43% yield) as a white solid. LCMS m/z 243.1 [M+H]⁺.

### Intermediate 17/5-((5-methyl-1H-pyrazol-3-yl)ethynyl)-1,3,4-thiadiazol-2-amine

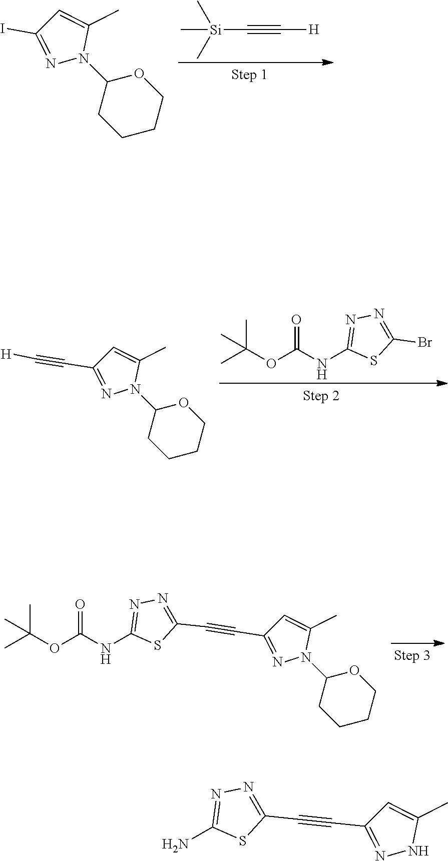

### Step 1/3-ethynyl-5-methyl-1-(tetrahydro-2H-pyran-2-yl)-1H-pyrazole

N₂ was bubbled through a solution of 3-iodo-5-methyl-1-tetrahydropyran-2-yl-pyrazole (1.20 g, 4.11 mmol), ethynyl(trimethyl)silane (4.70 mL, 33.3 mmol), Et₃N (4.70 mL, 33.7 mmol) in dry DMF (3.0 mL) while sonicating for 15 min. Pd(PPh₃)₄ (492 mg, 426 μmol) was added and the resulting mixture was stirred at 55 °C for 48 hrs. The yellow suspension was cooled to rt, filtered and the solids washed with Et₃N once. The filtrate was concentrated in vacuo and the residue was dissolved in MeOH (5 mL). K₂CO₃ (1.70 g, 12.3 mmol) was added, and the reaction mixture was stirred at rt for 6 hrs. The resulting mixture was filtered and concentrated in vacuo. The residue (dry pack) was purified by silica gel chromatography eluting with a gradient of EtOAc (5 to 30%) in Heptane. Appropriate fractions were combined and concentrated in vacuo to afford 3-ethynyl-5-methyl-1-(tetrahydro-2H-pyran-2-yl)-1H-pyrazole (585 mg, 74% yield).

### Step 2/tert-butyl (5-((5-methyl-1-(tetrahydro-2H-pyran-2-yl)-1H-pyrazol-3-yl)ethynyl)-1,3,4-thiadiazol-2-yl)carbamate

N₂ was bubbled through a solution of tert-butyl N-(5-bromo-1,3,4-thiadiazol-2-yl)carbamate (1.72 g, 6.15 mmol), 3-ethynyl-5-methyl-1-tetrahydropyran-2-yl-pyrazole (585 mg, 3.08 mmol), and Et₃N (3.50 mL, 25.1 mmol) in dry DMF (3.0 mL) while sonicating for 15 min. Pd(PPh₃)₄ (375 mg, 325 μmol). The reaction mixture was stirred at 70 °C for 48 hrs. EtOAc and brine were added, and the organic phase was washed with brine (3×). The combined aqueous phases were back extracted with EtOAc (×3). The combined organic phases were dried over MgSO₄, filtered, and concentrated in vacuo. The residue (dry pack) was purified by silica gel chromatography eluting with a gradient of EtOAc (5 to 70%) in Heptane. Appropriate fractions were combined and concentrated in vacuo to afford tert-butyl (5-((5-methyl-1-(tetrahydro-2H-pyran-2-yl)-1H-pyrazol-3-yl)ethynyl)-1,3,4-thiadiazol-2-yl)carbamate (750 mg, 63% yield).

### Step 3/Intermediate 17

To a solution of tert-butyl (5-((5-methyl-1-(tetrahydro-2H-pyran-2-yl)-1H-pyrazol-3-yl)ethynyl)-1,3,4-thiadiazol-2-yl)carbamate (750 mg, 1.93 mmol) in dry DCM (5 mL) was added TFA (2.97 mL, 38.5 mmol). The reaction mixture was stirred at rt for 4 hrs. Volatiles were removed in vacuo and the residue was dissolved in EtOAc followed by addition of NaOH 2N until pH-9. The aqueous phase was back extracted with EtOAc (6×). The combined organic extracts were dried over MgSO₄, filtered, and evaporated to dryness. The residue (dry pack) was purified by silica gel chromatography eluting with a gradient of MeOH (0 to 20%) in DCM. Appropriate fractions were combined and concentrated in vacuo to afford Intermediate 17 (320 mg, 81% yield) as a light orange solid. LCMS m/z 206.1 [M+H]⁺.

### Intermediate 18/5-(cyclopropylethynyl)thiazol-2-amine

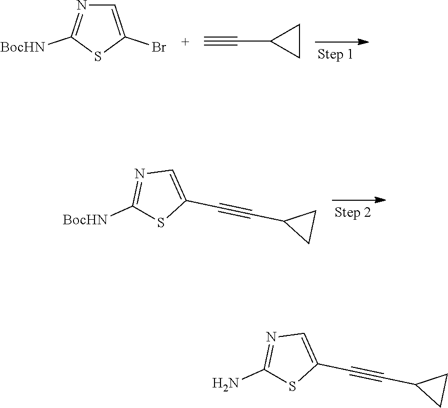

### Step 1/tert-butyl (5-(cyclopropylethynyl)thiazol-2-yl)carbamate

A pressure vessel was charged with tert-butyl (5-bromothiazol-2-yl)carbamate (20.0 g, 71.7 mmol) and DMF (200 mL). N₂ was bubbled through the solution for 20 min. Et₃N (30.0 mL, 215 mmol,) and CuI (1.38 g, 7.25 mmol) were added, followed by ethynylcyclopropane (36.0 mL, 425 mmol,) and finally Pd(PPh₃)₄ (8.42 g, 7.29 mmol). The vessel was capped and stirred overnight at 50 °C. The reaction mixture was cooled to rt, poured into saturated NH₄Cl, and extracted with EtOAc (3×). The combined organic layers were washed with brine, dried over Na₂SO₄, filtered, and concentrated in vacuo. The residue was purified by silica gel chromatography eluting with a gradient of EtOAc (5 to 80%) in heptane. Appropriate fractions were combined and concentrated in vacuo to afford tert-butyl (5-(cyclopropylethynyl)thiazol-2-yl)carbamate (11.7 g, 62% yield) as a light-yellow solid.

### Step 2/Intermediate 18

To a suspension of tert-butyl N-[5-(2-cyclopropylethynyl)thiazol-2-yl]carbamate (4.10 g, 15.5 mmol) in dry DCM (100 mL) was added TFA (12.0 mL, 156 mmol). The reaction mixture was stirred at rt for 2 hrs. Volatiles were removed in vacuo and the residue was taken in EtOAc. NaOH 2N was added until pH=9. The aqueous phase was extracted with EtOAc (×3). The combined organic layers were washed with brine, dried over Na₂SO₄, filtered, and concentrated in vacuo. The residue was purified by silica gel chromatography eluting with a gradient of MeOH (0 to 15%) in DCM. Appropriate fractions were combined and concentrated in vacuo to afford Intermediate 18 (1.60 g, 63% yield) as a brown-orange solid. LCMS m/z 165.1 [M+H]⁺.

### Intermediate 19/2-(difluoromethyl)-5-methoxy-4-(4,4,5,5-tetramethyl-1,3,2-dioxaborolan-2-yl)pyridine

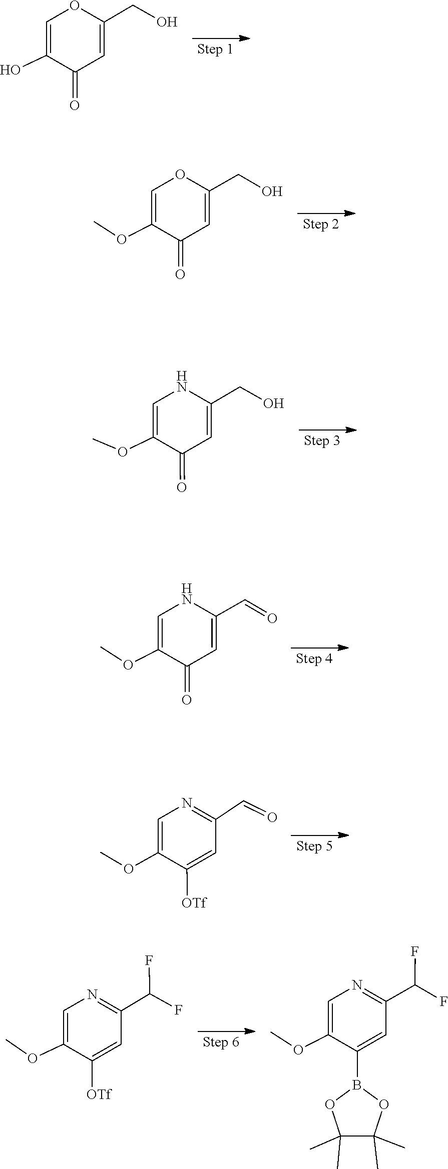

### Step 1/2-(hydroxymethyl)-5-methoxy-4H-pyran-4-one

To a suspension of Kojic acid (1000 g, 7.04 mol) in water (2.0 V) at 25-35 °C. The reaction mass was cooled to 0-5 °C. A solution of KOH (473.8 g, 8.44 mol) in water (0.4 V) was added slowly over a period of 30 min at 0-10 °C. The reaction mixture was stirred for 30 min at same temperature. Then into it, Dimethyl sulphate (736 mL, 7.76 mol) was added over a period of 45 min at the same temperature. The reaction mixture was stirred for 16 h at rt, the precipitate was filtered, washed with cold water (2.0 V) and dried under vacuum to afford 2-(hydroxymethyl)-5-methoxy-4H-pyran-4-one (781 g, 71% yield) as a white solid that was used in the next step without any further purification. ¹H NMR (400 MHz, DMSO-d₆) δ ppm: 8.11 (s, 1H), 6.31 (s, 1H), 5.73 (s, 1H), 4.32 (s, 2H), 3.66 (s, 3H). LCMS m/z 156.9 [M+H]⁺.

### Step 2/2-(hydroxymethyl)-5-methoxypyridin-4(1H)-one

2-(hydroxymethyl)-5-methoxy-4H-pyran-4-one (780 g, 5.00 mol) was added to a 30% aq. ammonia solution (6.0 V) at rt. The reaction mixture was heated to 85-90 °C over a period of 1.0 h and further stirred for 6 hrs at the same temperature. After completion of the reaction, the reaction mixture was cooled to rt and then volatiles were concentrated in vacuo and azeotroped with MeOH (2×2 V). To the residue in MeOH (10 V) was added activated charcoal (0.2 V) and the reaction mixture heated up to 60 °C and further stirred for 30 min at that temperature. The reaction mixture was cooled to 40-45 °C and filtered through a Celite bed. The solids were washed with MeOH (3 V). The filtrate was concentrated in vacuo to afford 2-(hydroxymethyl)-5-methoxypyridin-4(1H)-one (692 g, 89% yield). The residue was used in the next step without any further purification. ¹H NMR (400 MHz, DMSO-d₆) δ ppm: 11.15 (bs, 1H), 7.26 (bs, 1H), 6.07 (bs, 2H), 5.59 (bs, 2H), 4.34 (s, 3H). LCMS m/z 156.2 [M+H]⁺.

### Step 3/5-methoxy-4-oxo-1,4-dihydropyridine-2-carbaldehyde

To a solution of 2-(hydroxymethyl)-5-methoxypyridin-4(1H)-one (690 g, 4.90 mol) in 1,4-Dioxane (12 V) and MeOH (8 V) at 25-30 °C was added MnO₂ (6.96 kg, 80.05 mol). The reaction mixture was heated to 70-75 °C over a period of 1 h. and further stirred at the same temperature for 16 hrs. More MnO₂ (3.87 kg, 44.47 mol) and further stirred the reaction mixture at 70-75 °C for 5 h to complete the reaction. The reaction mixture was cooled to rt and then filtered through a Celite bed. The solids were washed with MeOH (10 V). The filtrate was concentrated in vacuo to afford 5-methoxy-4-oxo-1,4-dihydropyridine-2-carbaldehyde (525 g, 77%). The residue was used in the next step without any further purification. ¹H NMR (400 MHz, DMSO-d6) δ ppm: 10.99 (s, 1H), 9.81 (s, 1H), 8.40 (s, 1H), 7.35 (s, 1H), 4.06 (s, 3H). LCMS m/z 154.2 [M+H]⁺.

### Step 4/2-formyl-5-methoxypyridin-4-yl trifluoromethanesulfonate

To a solution of Et₃N (273 mL, 1.96 mol) in DCM (10 V) was added 5-methoxy-4-oxo-1,4-dihydropyridine-2-carbaldehyde (150 g, 0.98 mol) at rt. The reaction mixture was cooled to 0-5 °C. Triflic anhydride (198 mL, 1.18 mol) was added at the same temperature. The reaction mixture was further stirred for 30 min at the same temperature. After completion of the reaction, the reaction mixture was quenched slowly in ice cold water (10 V). Phases were separated and the aqueous layer was again extracted with DCM (5 V×2). The combined organic layers were washed with water (5 V), dried over Na₂SO₄, filtered, and concentrated in vacuo. The residue was purified by column chromatography eluting with a gradient of EtOAc (10 to 30%) in hexanes. Appropriate fractions were combined and concentrated in vacuo to afford pure 2-formyl-5-methoxypyridin-4-yl trifluoromethanesulfonate (112 g, 40% yield). ¹H NMR (400 MHz, DMSO-d6) δ ppm: 9.92 (s, 1H), 8.91 (s, 1H), 8.06 (s, 1H), 4.17 (s, 3H). LCMS m/z 286.0 [M+H]⁺.

### Step 5/2-(difluoromethyl)-5-methoxypyridin-4-yl trifluoromethanesulfonate

To a solution of 5-methoxypyridin-4-yl trifluoromethanesulfonate (100 g, 0.350 mol) in DCM (10 V) cooled at 0-5 °C was added DAST (46.3 mL, 1.05 mol) over a period of 45 min. The reaction mixture was further stirred for 2 h at the same temperature. After completion of the reaction, the reaction mixture was quenched slowly in ice cold water (2.0 L). The reaction mass was allowed to warm to rt and phases were separated. The aqueous layer was again extracted with DCM (0.5 L×2). The combined organic layers were washed with water (0.5 L), dried over Na₂SO₄, filtered, and concentrated in vacuo. The residue was purified by column chromatography eluting with a gradient of EtOAc (8 to 15%) in hexanes. Appropriate fractions were combined and concentrated in vacuo to afford pure 2-(difluoromethyl)-5-methoxypyridin-4-yl trifluoromethanesulfonate (76.0 g, 70% yield). ¹H NMR (400 MHz, DMSO-d₆) δ ppm: 8.83 (s, 1H), 7.95 (s, 1H), 7.15-6.87 (m, 1H), 4.11 (s, 3H). LCMS m/z 308.0 [M+H]⁺.

### Step 6/Intermediate 19

To a solution of 2-(difluoromethyl)-5-methoxypyridin-4-yl trifluoromethanesulfonate (50.0 g, 0.160 mol) in toluene (500 mL) at rt was added bis(pinacolato)diboron (53.81 g, 0.210 mol) and KOAc (56.0 g, 0.570 mol). The reaction mixture was degassed with N₂ gas over a period of 30 min. Then PdCl₂(dppf)·DCM (13.31 g, 16.30 mmol) was added and the reaction mixture was heated to 90-95 °C over a period of 30 min and further stirred for 16 h at the same temperature. The reaction mixture was cooled to rt and diluted with EtOAc (1 L). The reaction mixture was stirred for 45 min and then the reaction mixture was filtered through a Celite bed. The solids were washed with EtOAc (500 mL). The filtrate was concentrated in vacuo The residue was purified by silica gel chromatography eluting with a gradient of EtOAc (5 to 10%) in hexanes. Appropriate fractions were combined and concentrated in vacuo to afford Intermediate 19 (45.0 g, 96% yield) that was further purified with pentane washes. ¹H NMR (400 MHz, DMSO-d₆) δ ppm: 8.45 (s, 1H), 7.69 (s, 1H), 7.05-6.77 (m, 1H), 3.92 (s, 3H), 1.28 (s, 12H). LCMS m/z 203.8 (−Pinacol).

### Intermediate 20/methyl 4-bromo-2-(2-(difluoromethyl)-5-methoxypyridin-4-yl)benzoate

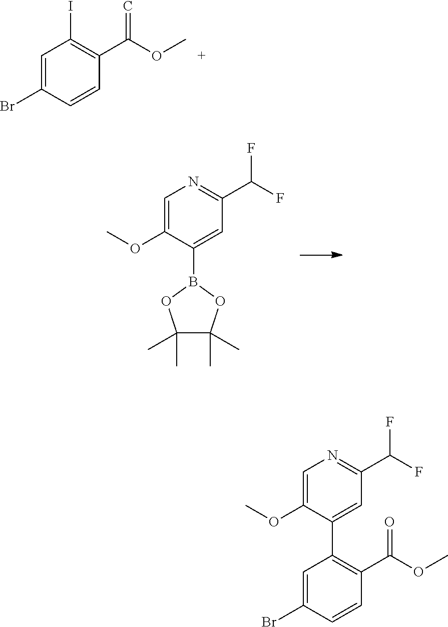

A vessel was charged with methyl 4-bromo-2-iodobenzoate (7.0 g, 20.5 mmol), Intermediate 19 (5.85 g, 20.5 mmol), Pd(dppf)Cl₂ (751.1 mg, 1.03 mmol), dioxane (110 mL) and aqueous K₂CO₃ (2 M, 26.0 mL). The vessel was stirred under N₂ at 80 °C for 1 h. The reaction mixture was cooled down then diluted with EtOAc and H₂O. The aqueous phase was extracted with EtOAc (2×). The combined organic layers were washed with brine, dried over Na₂SO₄, filtered, and concentrated in vacuo. The residue was purified by silica gel chromatography eluting with a gradient of EtOAc (0 to 50%) in heptane. Appropriate fractions were combined and concentrated in vacuo to afford Intermediate 20 (4.67 g, 61% yield) as a light-yellow solid. ¹H NMR (CDCl₃) δ: 8.29 (s, 1H), 7.85 (d, J=8.4 Hz, 1H), 7.64 (dd, J=8.4, 2.0 Hz, 1H), 7.50 (s, 1H), 7.46 (d, J=2.0 Hz, 1H), 6.66 (t, J=55.7 Hz, 1H), 3.87 (s, 3H), 3.68 (s, 3H). LCMS m/z 372.2 [M+H]⁺.

### Intermediate 21/methyl 6-chloro-2′-(difluoromethyl)-5′-methoxy-[4,4′-bipyridine]-3-carboxylate

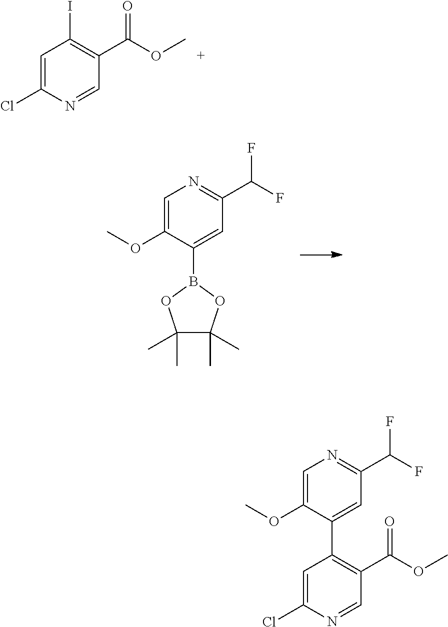

A vessel was charged with methyl 6-chloro-4-iodonicotinate (15.0 g, 50.42 mmol) and Intermediate 19 (15.23 g, 53.42 mmol). Dioxane (200 mL) and aqueous K₂CO₃ (2 M, 63.0 mL, 126 mmol) were added. N₂ was bubbled through the mixture for 10 min, then Pd(dppf)Cl₂·DCM (4.12 g, 5.04 mmol) was added and N₂ was bubbled through for 5 min. The reaction mixture was heated at 80 °C for 1 h 30 min. The reaction mixture was cooled down and passed through a Celite pad. The solids were filtered, washed with EtOAc (500 mL). The organic layer was separated, diluted with water, washed with brine, dried over Na₂SO₄, filtered, and concentrated in vacuo. The residue was purified by silica gel chromatography eluting with a gradient of EtOAc (5 to 80%) in hexanes. Appropriate fractions were combined and concentrated in vacuo to afford Intermediate 21 (15.5 g, 93.5% yield) as a brown solid. ¹H NMR (400 MHz, DMSO-d₆) δ 8.84 (s, 1H), 8.55 (s, 1H), 7.74 (s, 2H), 6.97 (t, J=55.0 Hz, 1H), 3.87 (s, 3H), 3.69 (s, 3H). LCMS m/z 329.0 [M+H]⁺.

### Intermediate 22/methyl 5-bromo-1-((5-methyl-1,3,4-oxadiazol-2-yl)methyl)-2-oxo-1,2-dihydropyridine-4-carboxylate

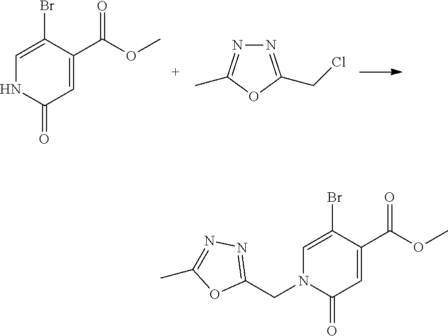

To a suspension of methyl 5-bromo-2-oxo-1,2-dihydropyridine-4-carboxylate (25.0 g, 107.7 mmol) and 2-(chloromethyl)-5-methyl-1,3,4-oxadiazole (15.0 g, 113.1 mmol) in dry MeCN (403 mL) at 0 °C was added CsCO₃ (70.5 g, 217 mmol) in one portion. The reaction mixture was slowly warmed at rt overnight in the cold bath. The solids were filtered on a Celite pad, washed with MeCN (×3) and the filtrate concentrated in vacuo. The residue was purified by silica gel chromatography eluting with a gradient of Acetone (0 to 100%) in DCM. Appropriate fractions were combined and concentrated in vacuo to afford Intermediate 22 (13.0 g, 37% yield) as a white solid. ¹H NMR (400 MHz, DMSO-d₆) δ 8.28 (d, J=0.6 Hz, 1H), 6.77 (d, J=0.5 Hz, 1H), 5.30 (s, 2H), 3.82 (s, 3H), 2.44 (s, 3H). LCMS m/z 329.9 [M+H]⁺.

### Intermediate 23/6-chloro-N-(5-(cyclopropylethynyl)-1,3,4-thiadiazol-2-yl)-2′-(difluoromethyl)-5′-methoxy-[4,4′-bipyridine]-3-carboxamide

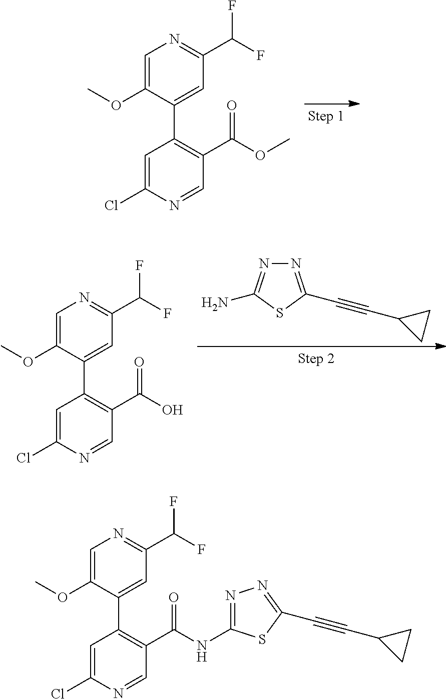

### Step 1/6-chloro-2′-(difluoromethyl)-5′-methoxy-[4,4′-bipyridine]-3-carboxylic acid

To a solution of Intermediate 21 (2.0 g, 5.84 mmol) in MeOH (8 mL), dioxane (20 mL) was added a solution of LiOH·H₂O (490 mg, 11.68 mmol) in water (6 mL) and heated to 60 °C for 15 min. Volatiles were removed in vacuo, 10 mL water was added followed by dropwise addition of HCl (2 M, 5.9 mL). The precipitate was filtered to afford 6-choro-2′-(difluoromethyl)-5′-methoxy-[4,4′-bipyridine]-3-carboxylic acid (1.84 g, 100% yield) as a beige solid.

### Step 2/Intermediate 23

To a solution of Intermediate 9 (1.16 g, 7.02 mmol) and 6-chloro-2′-(difluoromethyl)-5′-methoxy-[4,4′-bipyridine]-3-carboxylic acid (1.84 g, 5.85 mmol) in pyridine (15 mL) was added EDC (2.24 g, 11.7 mmol). The mixture was stirred for 2 hrs at rt. Volatiles were removed in vacuo and the residue was diluted with water. The mixture was extracted twice with DCM. Combined organic phases were dried over Na₂SO₄, filtered, and concentrated in vacuo. The residue was purified by silica gel chromatography eluting with a gradient of EtOAc (20 to 85%) in hexanes. Appropriate fractions were combined and concentrated in vacuo to afford Intermediate 23 (1.15 g, 42% yield) as a white solid. ¹H NMR (400 MHz, DMSO-d₆) δ 13.57 (br s, 1H), 8.80 (d, J=19.4 Hz, 1H), 8.42 (d, J=19.7 Hz, 1H), 7.77 (s, 2H), 6.96 (t, J=55.1 Hz, 1H), 5.74 (s, 1H), 3.63 (s, 3H), 1.75-1.57 (m, 1H), 1.02-0.90 (m, 2H), 0.88-0.80 (m, 2H). LCMS m/z 462.1 [M+H]⁺.

### Intermediate 24/tert-butyl 6-chloro-2′-(difluoromethyl)-5′-methoxy-[4,4′-bipyridine]-3-carboxylate

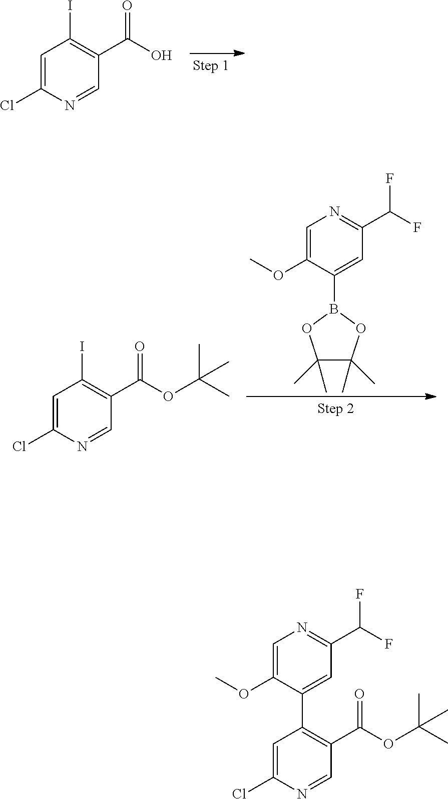 [SMILES: O=C(O)c1cnc(Cl)cc1I ; CC(C)(C)OC(=O)c1cnc(Cl)cc1I ; COc1cnc(C(F)F)cc1B1OC(C)(C)C(C)(C)O1 ; COc1cnc(C(F)F)cc1-c1cc(Cl)ncc1C(=O)OC(C)(C)C]

### Step 1/tert-butyl 6-chloro-4-iodonicotinate

To a solution of tert-butoxycarbonyl tert-butyl carbonate (18.48 g, 84.67 mmol) and 6-chloro-4-iodonicotinic acid (12.0 g, 42.3 mmol) in THF (200 mL) was added DMAP (1.03 g, 8.47 mmol). The mixture was stirred overnight at reflux. The reaction mixture was concentrated, and the residue was purified by silica gel chromatography eluting with a gradient of EtOAc (0 to 30%) in heptane to provide tert-butyl 6-chloro-4-iodonicotinate (11.18 g, 78% yield) as a white solid.

### Step 2/Intermediate 24

To a solution of tert-butyl 6-chloro-4-iodonicotinate (8.00 g, 23.6 mmol) in dioxane (80 mL) were added Intermediate 19 (7.06 g, 24.8 mmol), Pd(dppf)Cl₂ (1.72 g, 2.36 mmol) and aqueous K₂CO₃ (2 M, 23 mL). The mixture was degassed in vacuo and then backfilled with N₂ (×3). The mixture was stirred at 80 °C under N₂ for 30 min. The reaction mixture was concentrated to a small volume in vacuo, diluted with water, extracted with EtOAc (3×50 mL). The combined organic extracts were washed with brine, dried over Na₂SO₄, filtered. The filtrate was concentrated to dryness. The residue was purified by silica gel chromatography eluting with a gradient of EtOAc (20 to 100%) in heptane to provide Intermediate 24 (6.96 g, 80% yield) as an off-white solid. ¹H NMR (400 MHz, DMSO-d₆) δ 8.81 (d, J=0.6 Hz, 1H), 8.56 (s, 1H), 7.72-7.63 (m, 2H), 6.97 (t, J=54.9 Hz, 2H), 3.88 (s, 3H), 1.22 (s, 9H). LCMS m/z 371.3 [M+H]⁺.

### Intermediate 25/benzyl 6-chloro-2′-(difluoromethyl)-5′-methoxy-[4,4′-bipyridine]-3-carboxylate

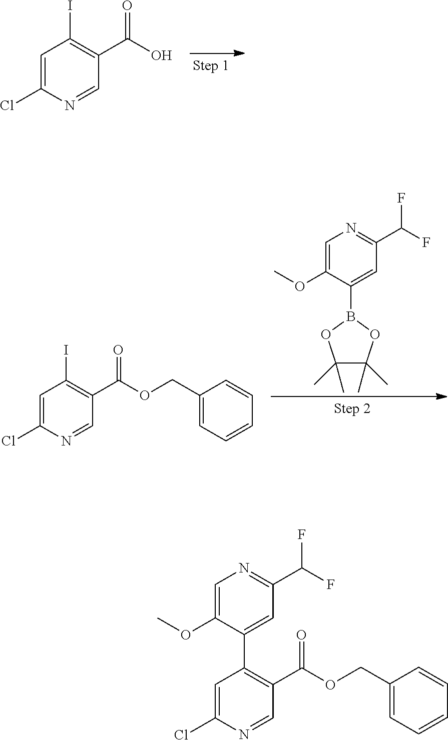 [SMILES: O=C(O)c1cnc(Cl)cc1I ; O=C(OCc1ccccc1)c1cnc(Cl)cc1I ; COc1cnc(C(F)F)cc1B1OC(C)(C)C(C)(C)O1 ; COc1cnc(C(F)F)cc1-c1cc(Cl)ncc1C(=O)OCc1ccccc1]

### Step 1/benzyl 6-chloro-4-iodonicotinate

To a solution of 6-chloro-4-iodonicotinic acid (5.00 g, 17.6 mmol) in DMF (30 mL) was added K₂CO₃ (4.88 g, 35.3 mmol) followed by Benzyl bromide (2.50 mL, 21.0 mmol). The mixture was stirred at rt for 3.5 h. The mixture was added slowly to 0.6 L of rapidly stirring water. The cloudy mixture was stirred for 30 min then filtered and dried on high vacuum to afford benzyl 6-chloro-4-iodonicotinate (6.09 g, 92% yield). ¹H NMR (Chloroform-d) δ: 8.78 (s, 1H), 8.01 (s, 1H), 7.48-7.36 (m, 5H), 5.40 (s, 2H).

### Step 2/Intermediate 25

N₂ was bubbled through a biphasic mixture of benzyl 6-chloro-4-iodonicotinate (6.07 g, 16.3 mmol), Intermediate 19 (4.94 g, 17.3 mmol) In aqueous K₂CO₃ (2 M, 20.5 mL) and dioxane (80 mL) for 30 min. Pd(dppf)Cl₂ (1.19 g, 1.62 mmol) was added, and the mixture stirred at 80 °C for 35 min. The reaction mixture was cooled, diluted with EtOAc and water and filtered through a Celite pad, rinsed with 100 mL EtOAc. The organic layer was separated, diluted with water, washed with brine, dried over Na₂SO₄, filtered, and concentrated in vacuo. The residue was purified by silica gel chromatography eluting with a gradient of EtOAc (0 to 30%) in hexanes. Appropriate fractions were combined and concentrated in vacuo to afford Intermediate 25 (4.74 g, 72% yield) as a beige solid. ¹H NMR (400 MHz, DMSO-d₆) δ 8.89 (d, J=0.6 Hz, 1H), 8.41 (s, 1H), 7.74-7.71 (m, 2H), 7.33-7.29 (m, 3H), 7.14-7.09 (m, 2H), 6.95 (t, J=55.0 Hz, 1H), 5.15 (s, 2H), 3.73 (s, 3H). LCMS m/z 405.1 [M+H]⁺.

### Intermediate 26/5-((1S,2S)-2-ethynylcyclopropyl)-1,3,4-thiadiazol-2-amine

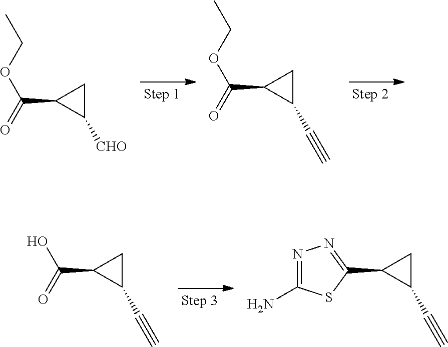

### Step 1/ethyl (1S,2S)-2-ethynylcyclopropane-1-carboxylate

To a stirred solution of ethyl (1S,2S)-2-formylcyclopropanecarboxylate (trans, chiral) (25.9 g, 182 mmol) in MeOH (245 mL) was added K₂CO₃ (50.4 g, 365 mmol) at 0 °C temperature. Reaction mixture was stirred at same temperature for 5 min. Dimethyl (1-diazo-2-oxopropyl)phosphonate solution, ~10% in CH₃CN (33.0 mL, 220 mmol) was added dropwise. The reaction mixture was warmed to RT and stirred for 2 h. After completion, the reaction mixture was concentrated, quenched with water (30 mL) and extracted with EtOAc (3×50 mL). The combined organic layers were dried over anhydrous Na₂SO₄ and concentrated under vacuum. The residue was used in the next step assuming 100% yield.

### Step 2/(1S,2S)-2-ethynylcyclopropane-1-carboxylic acid

To the solution of, ethyl (1S,2S)-2-ethynylcyclopropanecarboxylate (25.2 g, 182 mmol) in dioxane (230 mL), MeOH (57 mL) was added LiOH·H₁₂₀ (15.3 g, 365 mmol). The reaction mixture was stirred for 3 hrs at RT. The reaction mixture was concentrated under vacuum, acidified to pH 3 with HCl 1N then extracted with EtOAc (3×50 mL). The combined organic layers were dried over anhydrous Na₂SO₄, filtered and concentrated under vacuum up to 30% of total volume. The resulting solution of (1S,2S)-2-ethynylcyclopropanecarboxylic acid was used as such for the next step considering 100% yield. LCMS: m/z 108.7 [M−H]⁻.

### Step 3/Intermediate 26

T3P 50% wt in EtOAc (273 mL, 458 mmol, 50% purity) was added to a mixture of (1S,2S)-2-ethynylcyclopropanecarboxylic acid (20.08 g, 182.4 mmol) and thiosemicarbazide (19.94 g, 218.8 mmol) at RT. The resulting mixture was stirred at 90 °C overnight. The reaction mixture was cooled to RT, poured in aq. saturated NaHCO₃ and extracted with EtOAc (3×). The combined organic layers were washed with brine, dried over Na₂SO₄, filtered, and concentrated in vacuo. The residue was purified by silica gel chromatography eluting with a gradient of acetone (5 to 100%) in heptane. Appropriate fractions were combined and concentrated in vacuo to afford Intermediate 26 (13.4 g, 45% yield) as a white solid. ¹H NMR (400 MHz, MeOD) δ 2.49-2.45 (m, 1H), 2.32 (d, J=1.6 Hz, 1H), 1.86-1.83 (m, 1H), 1.49-1.36 (m, 2H). LCMS m/z 166.1 [M+H]⁺.

### Intermediate 27/tert-butyl 4-bromo-2-(2-(difluoromethyl)-5-methoxypyridin-4-yl)benzoate

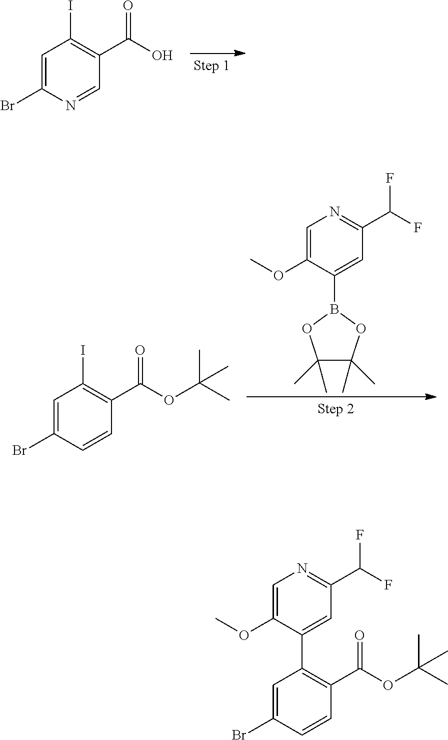 [SMILES: O=C(O)c1cnc(Br)cc1I ; CC(C)(C)OC(=O)c1ccc(Br)cc1I ; COc1cnc(C(F)F)cc1B1OC(C)(C)C(C)(C)O1 ; COc1cnc(C(F)F)cc1-c1cc(Br)ccc1C(=O)OC(C)(C)C]

### Step 1/tert-butyl 4-bromo-2-iodobenzoate

To a solution of tert-butoxycarbonyl tert-butyl carbonate (20.03 g, 91.8 mmol) and 4-bromo-2-iodo-benzoic acid (15.0 g, 45.9 mmol) in THF (200 mL) was added DMAP (1.12 g, 9.18 mmol). The mixture was stirred at 55 °C overnight. The reaction mixture was concentrated. The residue was purified by silica gel chromatography eluting with a gradient of EtOAc (0 to 20%) in heptane to provide tert-butyl 4-bromo-2-Iodo-benzoate (15.73 g, 89.5% yield).

### Step 2/Intermediate 27

A suspension of tert-butyl 4-bromo-2-iodo-benzoate (5.00 g, 13.1 mmol), Intermediate 19 (3.72 g, 13.1 mmol), Pd(dppf)Cl₂ (953 mg, 1.30 mmol), dioxane (70 mL) and aqueous K₂CO₃ (2 M, 16.40 mL) was flushed with N₂ and stirred at 80 °C overnight. The reaction mixture was diluted with EtOAc/water and filtered through Celite. The organic layer was separated, washed with brine, dried over Na₂SO₄, filtered, and concentrated in vacuo. The residue was purified by silica gel chromatography eluting with a gradient of EtOAc (0 to 50%) in hexanes. Appropriate fractions were combined and concentrated in vacuo to afford Intermediate 27 (4.11 g, 76% yield) as a beige solid. LCMS m/z 416.1 [M+H]⁺.

### Intermediate 28/methyl 2-(2-(difluoromethyl)-5-methoxypyridin-4-yl)-4-(methylamino)benzoate

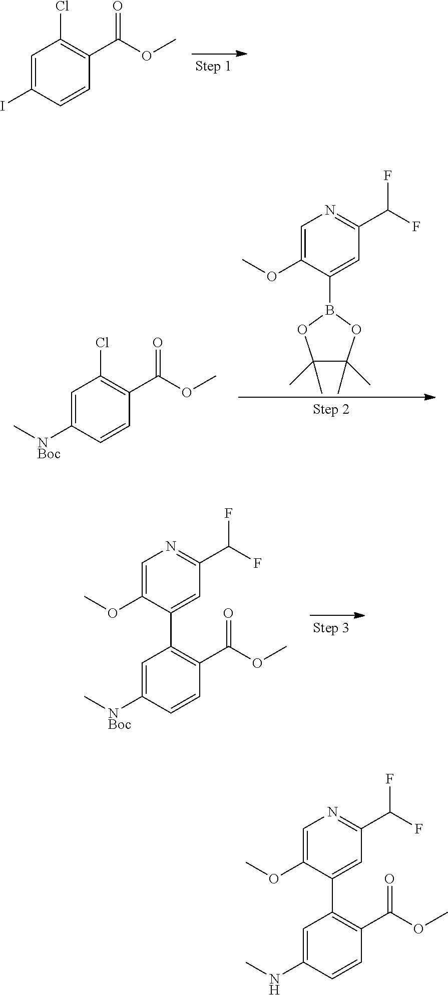

### Step 1/methyl 4-((tert-butoxycarbonyl)(methyl)amino)-2-chlorobenzoate

N₂ was bubbled through a mixture of methyl 2-chloro-4-iodobenzoate (7.8 g, 26.3 mmol), tert-butyl N-methylcarbamate (5.26 g, 40.1 mmol) and Cesium carbonate (5.49 g, 16.9 mmol) in dry Toluene (100 mL) while sonicating for 15 min. Pd(OAc)₂ (605 mg, 2.69 mmol) was added and the reaction mixture stirred at 90 °C under N₂ overnight. The suspension was filtered through a Celite pad, solids were washed with EtOAc, and the filtrate concentrated in vacuo. The residue was purified by silica gel chromatography eluting with a gradient of EtOAc (0 to 50%) in Heptane. Appropriate fractions were combined and concentrated in vacuo to afford methyl 4-((tert-butoxycarbonyl)(methyl)amino)-2-chlorobenzoate (6.90 g, 87% yield) as a dark yellow oil.

### Step 2/methyl 4-((tert-butoxycarbonyl)(methyl)amino)-2-(2-(difluoromethyl)-5-methoxypyridin-4-yl)benzoate

N₂ was bubbled for 10 min in a mixture of methyl 4-((tert-butoxycarbonyl)(methyl)amino)-2-chlorobenzoate (1.80 g, 6.01 mmol), Intermediate 19 (1.91 g, 6.70 mmol) and K₂CO₃ (1.90 g, 13.75 mmol) in H₂O (4 mL) and 1-4-dioxane (13 mL). To the resulting mixture was added Pd(OAc)₂ (136 mg, 606 μmol) and SPhos (492 mg, 1.20 mmol) and the resulting mixture was stirred at 90 °C for 2.5 h. The reaction mixture was cooled to rt, diluted with EtOAc and H₂O, filtered on a Celite bed. Solids were washed with EtOAc. Layers were separated, aqueous layer was back extracted with EtOAc (2×). The combined organic layers were washed with brine, dried over Na₂SO₄, filtered, and concentrated in vacuo. The residue was purified by silica gel chromatography eluting with a gradient of EtOAc (5 to 80%) in Heptane. Appropriate fractions were combined and concentrated in vacuo to afford methyl 4-((tert-butoxycarbonyl)(methyl)amino)-2-(2-(difluoromethyl)-5-methoxypyridin-4-yl)benzoate (2.41 g, 95% yield) as an amber gum.

### Step 3/Intermediate 28

To a solution of methyl 4-((tert-butoxycarbonyl)(methyl)amino)-2-(2-(difluoromethyl)-5-methoxypyridin-4-yl)benzoate (2.21 g, 5.23 mmol) in DCM (7.5 mL) was added Hydrogen Chloride 4M in dioxane (15 mL, 60 mmol). The mixture was stirred at rt for 1 h 50 min. The reaction mixture was concentrated to dryness to afford Intermediate 28 (2.02 g, 96% yield) as a yellow foamy solid (HCl salt). ¹H NMR (400 MHz, DMSO-d6) δ 8.41 (s, 1H), 7.72 (d, J=8.7 Hz, 1H), 7.41 (s, 1H), 6.93 (t, J=55.1 Hz, 1H), 6.60 (dd, J=8.7, 2.4 Hz, 1H), 6.34 (d, J=2.4 Hz, 1H), 3.81 (s, 3H), 3.51 (s, 3H), 2.73 (s, 3H). LCMS m/z 416.1 [M+H]⁺.

### Intermediate 29/methyl 6-chloro-4-(2-methoxy-5-(trifluoromethyl)phenyl)nicotinate

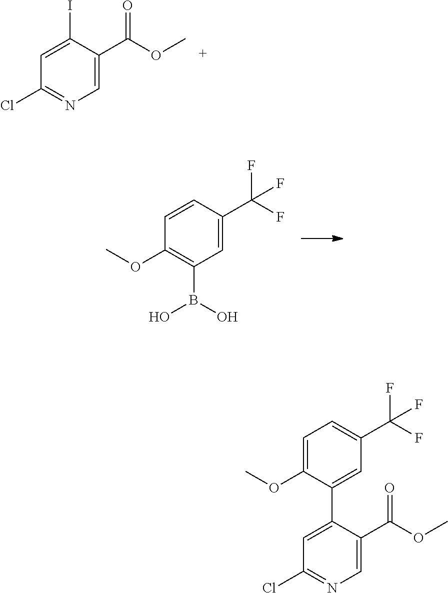

N₂ was bubbled through a biphasic mixture of methyl 6-chloro-4-iodonicotinate (2.0 g, 6.72 mmol), (2-methoxy-5-(trifluoromethyl)phenyl)boronic acid (1.50 g, 6.82 mmol) and K₂CO₃ (2.79 g, 20.17 mmol) in 1,4-Dioxane (20 mL)/water (8 mL) while sonicating for 15 min. Pd(dppf)Cl₂·DCM (549 mg, 0.67 mmol) was then added. The reaction mixture was stirred at 50 °C for 1 h 15 minutes. Volatiles were removed in vacuo. The residue was purified by silica gel chromatography eluting with a gradient of EtOAc (5 to 35%) in Heptane. Appropriate fractions were combined and concentrated in vacuo to Intermediate 29 (1.95 g, 84% yield). ¹H NMR (400 MHz, DMSO-d₆) δ 8.73 (d, J=0.5 Hz, 1H), 7.78 (ddd, J=8.7, 2.4, 0.8 Hz, 1H), 7.71 (d, J=2.7 Hz, 1H), 7.65 (d, J=0.6 Hz, 1H), 7.23 (d, J=8.7 Hz, 1H), 3.72 (s, 3H), 3.63 (s, 3H). LCMS m/z 346.0 [M+H]⁺.

### Intermediate 30/methyl 4-bromo-2-(2-chloro-5-methoxypyridin-4-yl)benzoate

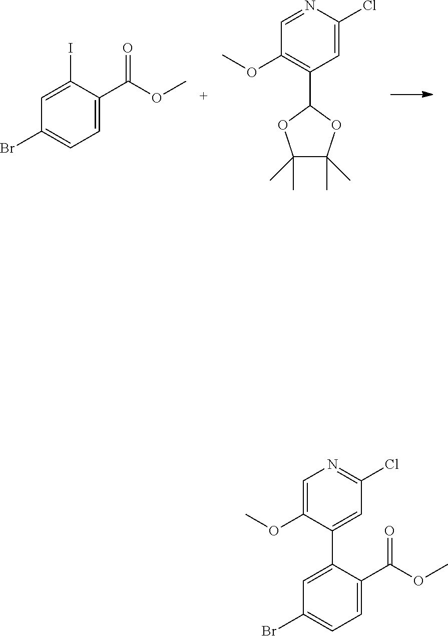

To a solution of methyl 4-bromo-2-iodobenzoate (1.0 g, 2.93 mmol) in dioxane (10 mL) were added aqueous Sodium carbonate (2 M, 3.0 mL), 2-chloro-5-methoxy-4-(4,4,5,5-tetramethyl-1,3-dioxolan-2-yl)pyridine (800.0 mg, 2.97 mmol) and Pd(dppf)Cl₂ (210 mg, 0.28 mmol) in dioxane (10 mL). The mixture was degassed in vacuo, backfilled with N₂. The mixture was stirred at 60 °C for 10 h. The volatiles were removed in vacuo. The residue was purified by silica gel chromatography eluting with a gradient of EtOAc (0 to 40%) in Heptane. Appropriate fractions were combined and concentrated in vacuo to afford Intermediate 30 (650 mg, 62% yield) as a white solid. ¹H NMR (400 MHz, DMSO-d6) δ 7.98 (s, 1H), 7.81 (d, J=8.4 Hz, 1H), 7.60 (ddd, J=8.5, 2.0, 0.6 Hz, 1H), 7.40 (d, J=2.0 Hz, 1H), 7.16 (d, J=0.5 Hz, 1H), 3.77 (s, 3H), 3.67 (d, J=0.5 Hz, 3H). LCMS m/z 356.2 [M+H]⁺.

### Intermediate 31/methyl 6-bromo-2-(dimethylcarbamoyl)imidazo[1,2-a]pyridine-7-carboxylate

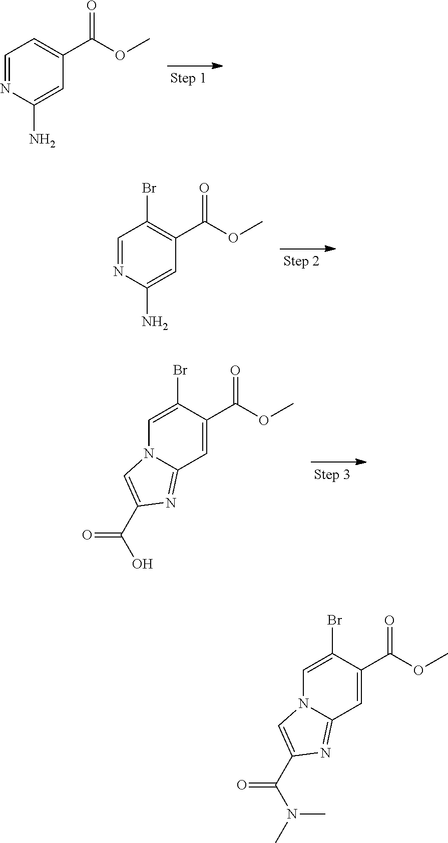 [SMILES: COC(=O)c1ccnc(N)c1 ; COC(=O)c1cc(N)ncc1Br ; COC(=O)c1cc2nc(C(=O)O)cn2cc1Br ; COC(=O)c1cc2nc(C(=O)N(C)C)cn2cc1Br]

### Step 1/methyl 2-amino-5-bromoisonicotinate

To a stirred solution of methyl 2-aminoisonicotinate (5.00 g, 32.9 mmol) in DMF (50 mL), was added NBS (6.43 g, 36.2 mmol) at rt. The reaction mixture was stirred at rt for 20 min. The reaction mixture was quenched with ice cold water (200 mL) and extracted with EtOAc (3×100 mL). The combined organic layers were collected, dried over anhydrous Na₂SO₄, filtered, and concentrated in vacuo. The residue was purified by silica gel chromatography eluting with a gradient of EtOAc (0 to 40%) in Heptane. Appropriate fractions were combined and concentrated in vacuo to afford methyl 2-amino-5-bromoisonicotinate (3.5 g, 46%). LCMS m/z 231.1 [M+H]⁺.

### Step 2/6-bromo-7-(methoxycarbonyl)imidazo[1,2-a]pyridine-2-carboxylic acid

To a stirred solution of methyl 2-amino-5-bromoisonicotinate (1.00 g, 4.33 mmol) and 3-bromo-2-oxopropanoic acid (0.870 g, 5.19 mmol) in DMF (5.0 mL), was added p-TSA (0.250 g, 1.24 mmol) under nitrogen. The reaction mixture was heated at 130 °C for 1.5 h. The reaction mixture was poured onto crushed ice. Solids were filtered and dried in vacuo to afford 6-bromo-7-(methoxycarbonyl)imidazo[1,2-a]pyridine-2-carboxylic acid (0.35 g, 27%). LCMS m/z 299.0 [M+H]⁺.

### Step 3/Intermediate 31

To a stirred solution of 6-bromo-7-(methoxycarbonyl)imidazo[1,2-a]pyridine-2-carboxylic acid (0.250 g, 0.840 mmol) in DMF (3.0 mL) was added HATU (0.950 g, 2.51 mmol) at 0 °C and stirred for 30 min. A 2 M solution of Dimethylamine in THF (0.5 mL, 1.0 mmol) and DIPEA (0.44 mL, 2.51 mmol) were then added. The reaction mixture was stirred at rt for 3 hrs. The reaction mixture was poured onto crushed ice and extracted with EtOAc (3×10 mL). The combined organic layers were collected, dried over anhydrous Na₂SO₄ and concentrated in vacuo. The residue was purified by aluminum oxide (basic) chromatography eluting with a gradient of EtOAc (0 to 70%) in Heptane. Appropriate fractions were combined and concentrated in vacuo to afford Intermediate 31 (0.13 g, 48%). LCMS m/z 326.0 [M+H]⁺.

### Intermediate 32/5-(cyclopropylethynyl)-4-methylthiazol-2-amine

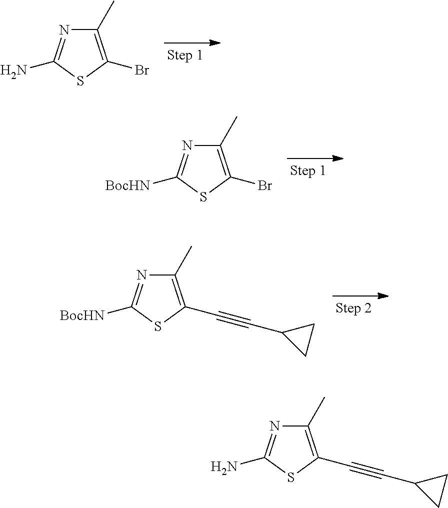

### Step 1/tert-butyl (5-bromo-4-methylthiazol-2-yl)carbamate

To a solution of 5-bromo-4-methylthiazol-2-amine (15.0 g, 77.70 mmol) in THF (150 mL) was added Triethylamine (15.72 g, 155.93 mmol) and DMAP (1.90 g, 15.54 mmol) followed by slow addition of Boc anhydride (20.35 g, 93.23 mmol) at 0 °C. The reaction was allowed to stir at room temperature for 16 h. The reaction mixture was poured into water (250 mL) and extracted with EtOAc (3×250 mL), the combined organic layer was dried over anhydrous Na₂SO₄ and concentrated in vacuo. The residue was purified by silica gel chromatography eluting with a gradient of EtOAc (0 to 50%) in heptanes. Appropriate fractions were combined and concentrated in vacuo to afford tert-butyl (5-bromo-4-methylthiazol-2-yl)carbamate (8.0 g, 35%). LCMS m/z 236.5 [M-tBu]⁺.

### Step 2/tert-butyl (5-(cyclopropylethynyl)-4-methylthiazol-2-yl)carbamate

A solution of tert-butyl (5-bromo-4-methylthiazol-2-yl)carbamate (7.0 g, 23.88 mmol), ethynylcyclopropane (6.31 g, 95.50 mmol) and N,N,N′,N′-tetramethylguanidine (3.02 g, 26.62 mmol) in DMF (70 mL) was degassed with N₂ gas for 15 min. Then PdCl₂(dppf)·DCM complex (0.97 g, 1.19 mmol) and CuI (0.45 g, 2.39 mmol) were added. Then the reaction mixture was heated to 90 °C (Pre-heated oil bath) for 1 h. The reaction mixture was poured into water (150 mL) and extracted with EtOAc (3×150 mL), the combined organic layer was dried over anhydrous Na₂SO₄ and concentrated in vacuo. The residue was purified by silica gel chromatography eluting with a gradient of EtOAc (0 to 40%) in heptanes. Appropriate fractions were combined and concentrated in vacuo to afford tert-butyl (5-(cyclopropylethynyl)-4-methylthiazol-2-yl)carbamate (2.2 g, 33%). LCMS m/z 278.8 [M+H]⁺.

### Step 3/Intermediate 32

To a solution of tert-butyl (5-(cyclopropylethynyl)-4-methylthiazol-2-yl)carbamate (2.2 g, 7.90 mmol) in DCM (22 mL) was added slowly TFA (11.0 mL) at 0 °C. The reaction was allowed to stir at the same temperature for 10 min and then at room temperature for 5 h. Volatiles were removed under vacuum at 40 °C. Then saturated NaHCO₃ solution (20 mL) was added under stirring and extracted with EtOAc (3×50 mL), the combined organic layer was dried over anhydrous Na₂SO₄ and concentrated in vacuo. The residue was purified by silica gel chromatography eluting with a gradient of EtOAc (0 to 70%) in heptanes. Appropriate fractions were combined and concentrated in vacuo to afford 5-(cyclopropylethynyl)-4-methylthiazol-2-amine (0.68 g, 48%) as light brown solid. ¹H NMR (400 MHz, DMSO d₆) δ 7.17 (s, 2H), 2.07 (s, 3H), 1.52 (bs, 1H), 8.54 (d, J=5.6 Hz, 2H), 0.67 (bs, 2H). LCMS m/z 179.3 [M+H]⁺.

### Intermediate 33/methyl 4-(cyanomethyl)-2-(2-(difluoromethyl)-5-methoxypyridin-4-yl)benzoate

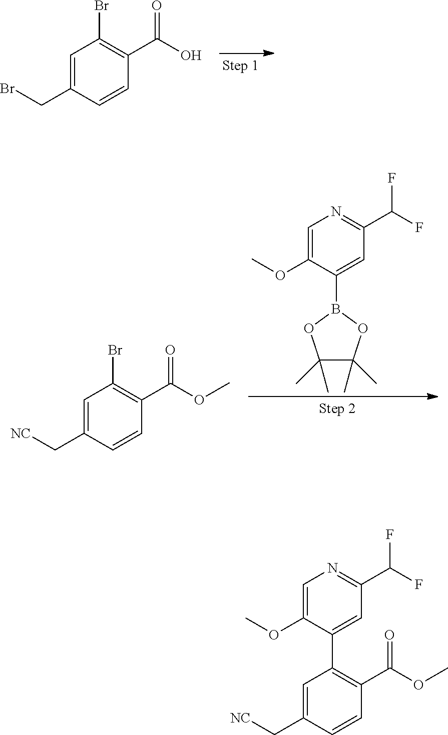

### Step 1/methyl 2-bromo-4-(cyanomethyl)benzoate

A mixture of 2-bromo-4-(bromomethyl)benzoic acid (10 g, 32.47 mmol) and tetrabutylammonium bromide (1.05 g, 3.26 mmol) in DCM (60 mL) and water (60 mL) was treated with potassium cyanide (6.34 g, 97.41 mmol) dissolved in water (60 mL). The mixture was stirred for 2 h at rt. The reaction mixture was diluted with DCM and the org layer was separated, dried over MgSO₄, filtered and concentrated. The residue was purified by silica gel chromatography eluting with a gradient of EtOAc (10 to 40%) in heptane to provide methyl 2-bromo-4-(cyanomethyl)benzoate (5.18 g, 63% yield). ¹H NMR (Chloroform-d) δ: 7.83 (d, J=8.0 Hz, 1H), 7.66 (dd, J=1.8, 0.9 Hz, 1H), 7.36 (ddt, J=8.0, 1.6, 0.8 Hz, 1H), 3.94 (s, 3H), 3.78 (t, J=0.8 Hz, 2H).

### Step 2/Intermediate 33

A suspension of methyl 2-bromo-4-(cyanomethyl)benzoate (1.0 g, 3.94 mmol), Intermediate 19 (1.12 g, 3.94 mmol), Pd(dppf)Cl₂ (144 mg, 0.2 mmol), dioxane (20 mL) and aqueous K₂CO₃ (2 M, 5.0 mL) was flushed with N₂ and stirred at 80 °C overnight. The reaction mixture was diluted with EtOAc/water and filtered through Celite. The organic layer was separated, diluted with water, washed with brine, dried over Na₂SO₄, filtered, and concentrated in vacuo. The residue was purified by silica gel chromatography eluting with a gradient of EtOAc (30 to 100%) in hexanes. Appropriate fractions were combined and concentrated in vacuo to afford Intermediate 33 (970 mg, 74% yield). ¹H NMR (Chloroform-d) δ: 8.30 (s, 1H), 8.01 (d, J=8.0 Hz, 1H), 7.53-7.46 (m, 2H), 7.26 (d, J=1.4 Hz, 1H), 6.67 (t, J=55.7 Hz, 1H), 3.86 (s, 3H), 3.85 (d, J=0.8 Hz, 2H), 3.69 (s, 3H). LCMS m/z 333.2 [M+H]⁺.

### Intermediate 34/6-(cyanomethyl)-2′-(difluoromethyl)-5′-methoxy-[4,4′-bipyridine]-3-carboxylic acid

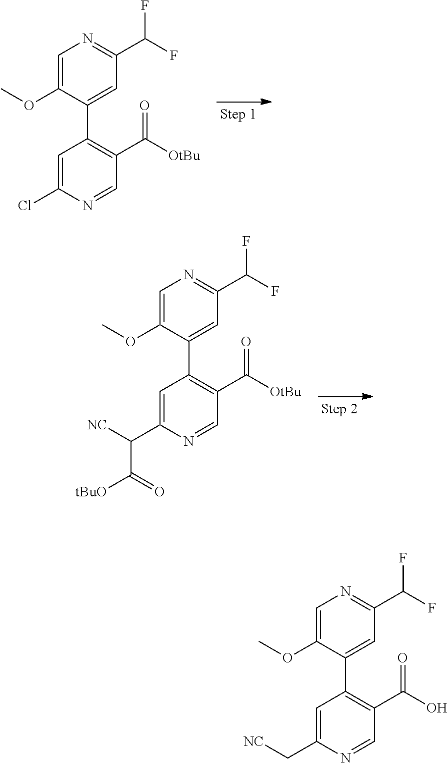 [SMILES: COc1cnc(C(F)F)cc1-c1cc(Cl)ncc1C(=O)OC(C)(C)C ; COc1cnc(C(F)F)cc1-c1cc(C(C#N)C(=O)OC(C)(C)C)ncc1C(=O)OC(C)(C)C ; COc1cnc(C(F)F)cc1-c1cc(CC#N)ncc1C(=O)O]

### Step 1/tert-butyl 6-(2-(tert-butoxy)-1-cyano-2-oxoethyl)-2′-(difluoromethyl)-5′-methoxy-[4,4′-bipyridine]-3-carboxylate

A mixture of Intermediate 24 (1.30 g, 3.51 mmol), tert-butyl cyanoacetate (0.740 g, 5.27 mmol) and K₂CO₃ (1.45 g, 10.5 mmol) in DMF (10 mL) was heated at 90 °C for 5 hrs. The reaction mixture was quenched in ice cold water (80 mL) and extracted with EtOAc (3×60 mL). The combined organic layers were washed with brine (50 mL) and dried over anhydrous Na₂SO₄, filtered and evaporated in vacuo. The residue was purified by silica gel chromatography eluting with a gradient of EtOAc (0 to 30%) in hexanes. Appropriate fractions were combined and concentrated in vacuo to afford tert-butyl 6-(2-(tert-butoxy)-1-cyano-2-oxoethyl)-2′-(difluoromethyl)-5′-methoxy-[4,4′-bipyridine]-3-carboxylate (1.10 g, 66%). LCMS m/z 476.3 [M+H]⁺.

### Step 2/Intermediate 34

To a solution of tert-butyl 6-(2-(tert-butoxy)-1-cyano-2-oxoethyl)-2′-(difluoromethyl)-5′-methoxy-[4,4′-bipyridine]-3-carboxylate (0.65 g, 1.37 mmol) in toluene (6.5 mL) was added Montmorillonite K10 (1.95 g) and the reaction mixture heated at 120 °C for 7 hrs. Volatiles were removed in vacuo. The residue was diluted with ethyl acetate (25 mL), filtered through a Celite pad, and washed with ethyl acetate (3×20 mL). The filtrate was evaporated in vacuo and the residue was triturated with n-pentane to afford Intermediate 34 (0.21 g, 48%). ¹H NMR (400 MHz, DMSO d₆) δ 13.28 (s, 1H), 8.97 (s, 1H), 8.51 (s, 1H), 7.60 (s, 1H), 7.44 (s, 1H), 6.95 (t, J=54.8 Hz, 1H), 4.33 (s, 2H), 4.0 (s, 3H). LCMS m/z 320.1 [M+H]⁺.

### Intermediate 35/benzyl 2-(2-(difluoromethyl)-5-methoxypyridin-4-yl)-4-(4,4,5,5-tetramethyl-1,3,2-dioxaborolan-2-yl)benzoate

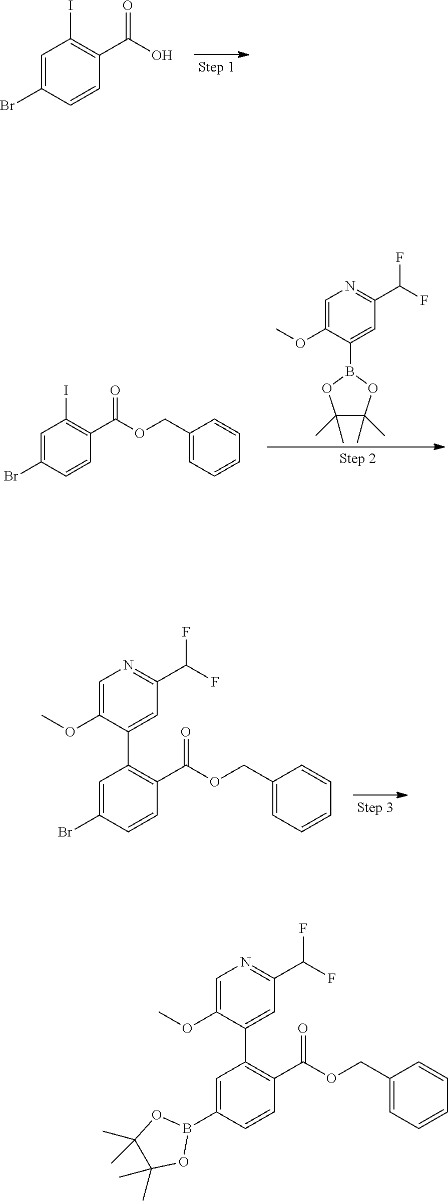 [SMILES: O=C(O)c1ccc(Br)cc1I ; O=C(OCc1ccccc1)c1ccc(Br)cc1I ; COc1cnc(C(F)F)cc1B1OC(C)(C)C(C)(C)O1 ; COc1cnc(C(F)F)cc1-c1cc(Br)ccc1C(=O)OCc1ccccc1 ; COc1cnc(C(F)F)cc1-c1cc(B2OC(C)(C)C(C)(C)O2)ccc1C(=O)OCc1ccccc1]

### Step 1/benzyl 4-bromo-2-iodobenzoate

To a solution of 4-bromo-2-iodo-benzoic acid (2.00 g, 6.12 mmol) and bromomethylbenzene (800 μL, 6.73 mmol) in DMF (20 mL) was added Na₂CO₃ (720 mg, 6.79 mmol). The reaction was stirred at rt for 18 hrs. The crude reaction mixture was concentrated to dryness in vacuo. The residue was purified on silica gel column eluting with a gradient of EtOAc (0-30%) in hexanes. Appropriate fractions were combined and concentrated in vacuo to afford benzyl 4-bromo-2-iodobenzoate (1.86 g, 73% yield) as a semi-solid.

### Step 2/benzyl 4-bromo-2-(2-(difluoromethyl)-5-methoxypyridin-4-yl)benzoate

A vessel was charged with benzyl 4-bromo-2-iodobenzoate (1.86 g, 4.46 mmol), Intermediate 19 (1.30 g, 4.56 mmol), Pd(dppf)Cl₂ (170 mg, 0.230 mmol), aqueous K₂CO₃ (2 M, 5.6 mL, 11.2 mmol) and dioxane (20 mL). The vessel was degassed in vacuo and then stirred under nitrogen at 80 °C for 1 h. The reaction mixture was diluted with water (30 mL), extracted with EtOAc (3×30 ml). The combined organic extracts were washed with water and brine consecutively, dried over Na₂SO₄, filtered and concentrated. The residue was purified by silica gel chromatography eluting with a gradient of EtOAc (0 to 30%) in heptane. Appropriate fractions were combined and concentrated in vacuo to afford benzyl 4-bromo-2-(2-(difluoromethyl)-5-methoxypyridin-4-yl)benzoate (1.38 g, 69% yield).

### Step 3/Intermediate 35

A mixture of benzyl 4-bromo-2-(2-(difluoromethyl)-5-methoxypyridin-4-yl)benzoate (200 mg, 0.450 mmol), 4,4,5,5-tetramethyl-2-(4,4,5,5-tetramethyl-1,3,2-dioxaborolan-2-yl)-1,3,2-dioxaborolane (169 mg, 666 μmol), aqueous KOAc (132 mg, 1.34 mmol), dioxane (4 mL) and Pd(dppf)Cl₂ (32.0 mg, 43.7 μmol). The vessel was stirred under nitrogen at 80 °C for 1 h. The reaction mixture was diluted with water (30 mL), extracted with EtOAc (3×30 ml). The combined organic extracts were washed with water and brine consecutively, dried over Na₂SO₄, filtered and concentrated. The residue was purified by silica gel chromatography eluting with a gradient of EtOAc (0 to 70%) in heptane to provide Intermediate 35 (166 mg, 75% yield). LCMS m/z 495.9 [M+H]*.

### Preparation of the Compounds of the Invention

### Compound 1/Method A/Racemic N-(5-((1R,2R)-2-(4-cyanophenyl)cyclopropyl)-1,3,4-thiadiazol-2-yl)-3-(5-fluoro-2-methoxyphenyl)isonicotinamide

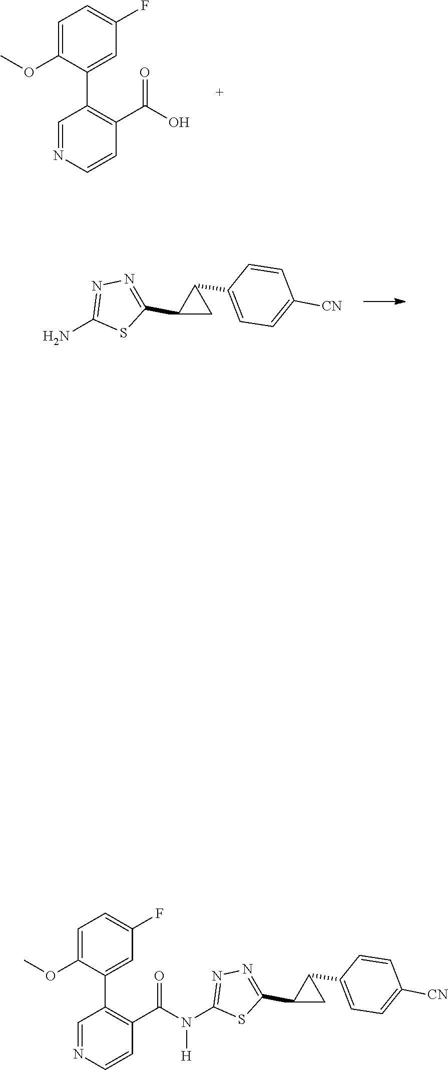

To a solution of Intermediate 2 (17.4 mg, 70.2 μmol) and Intermediate 16 (17.0 mg, 70.2 μmol) in pyridine (1 mL) was added EDC·HCl (26.9 mg, 140 μmol). The reaction mixture was stirred at 35 °C for 2 hrs. The crude reaction mixture was filtered, and the filtrate was purified by preparative HPLC (Phenomenex Gemini®) eluting with a gradient of CH₃CN (35 to 65%) in water both containing 0.1% formic acid. Appropriate fractions were combined and lyophilized to afford a racemic mixture of Compound 1 (8.0 mg, 24% yield). ¹H NMR (400 MHz, DMSO-d₆) δ 13.00 (s, 1H), 8.70 (dd, J=5.0, 1.1 Hz, 1H), 8.59 (d, J=1.4 Hz, 1H), 7.76-7.70 (m, 2H), 7.63 (dd, J=5.0, 1.3 Hz, 1H), 7.44-7.35 (m, 2H), 7.25 (dd, J=8.9, 3.2 Hz, 1H), 7.16 (td, J=8.6, 3.0 Hz, 1H), 6.93 (dd, J=9.0, 4.7 Hz, 1H), 3.42 (s, 3H), 2.84 (ddd, J=9.4, 5.9, 4.4 Hz, 1H), 2.68 (dd, J=9.1, 5.5 Hz, 1H), 1.77 (dt, J=9.4, 5.4 Hz, 1H), 1.69 (dt, J=9.5, 5.7 Hz, 1H). LCMS m/z 471.9 [M+H]⁺.

### Compound 2/Method A/Racemic N-(5-((1R,2R)-2-(4-cyanophenyl)cyclopropyl)-1,3,4-thiadiazol-2-yl)-3-(5-cyano-2-methoxyphenyl)isonicotinamide

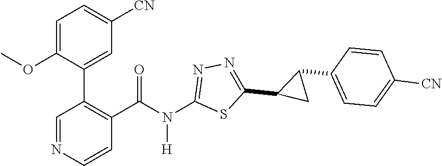

To a solution of Intermediate 3 (23.1 mg, 90.8 μmol) and 4-[(1R,2R)-2-(5-amino-1,3,4-thiadiazol-2-yl)cyclopropyl]benzonitrile previously described (17 mg, 70.2 μmol) in pyridine (1 mL) was added EDC·HCl (26.9 mg, 140 μmol). The reaction mixture was stirred at 35 °C for 2 hrs. The crude reaction mixture was filtered, and the filtrate was purified by preparative HPLC (Phenomenex Gemini®) eluting with a gradient of CH₃CN (35 to 65%) in water both containing 0.1% formic acid. Appropriate fractions were combined and lyophilized to afford a racemic mixture of Compound 2 (3.3 mg, 8.5% yield). ¹H NMR (400 MHz, DMSO-d₆) δ 13.09 (s, 1H), 8.78 (d, J=5.0 Hz, 1H), 8.66 (s, 1H), 7.88 (dt, J=6.8, 2.1 Hz, 2H), 7.76 (d, 2H), 7.71 (dd, J=5.0, 0.7 Hz, 1H), 7.43 (d, 2H), 7.16 (d, J=9.2 Hz, 1H), 3.56 (s, 3H), 2.88 (dt, J=9.2, 5.2 Hz, 1H), 2.71 (ddd, J=9.8, 6.1, 4.4 Hz, 1H), 1.80 (dt, J=9.0, 5.3 Hz, 1H), 1.73 (ddd, J=8.8, 6.2, 4.9 Hz, 1H). LCMS m/z 479.1 [M+H]⁺.

### Compound 3/Method A/Racemic 3-(5-fluoro-2-methoxyphenyl)-N-(5-((1R,2R)-2-(1-methyl-1H-pyrazol-3-yl)cyclopropyl)-1,3,4-thiadiazol-2-yl)isonicotinamide

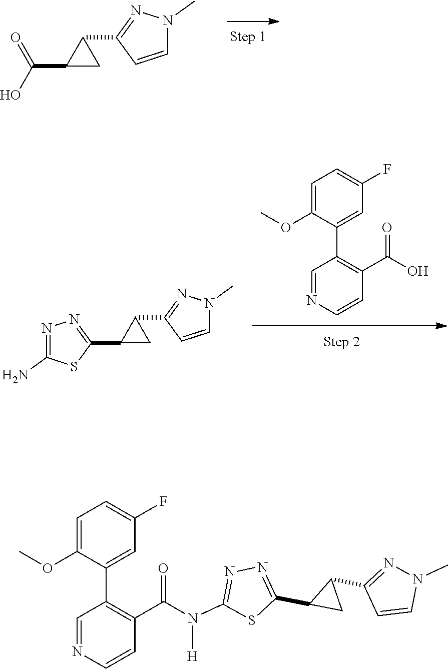

### Step 1/Racemic 5-((1R,2R)-2-(1-methyl-1H-pyrazol-3-yl)cyclopropyl)-1,3,4-thiadiazol-2-amine

Racemic (1R,2R)-2-(1-methyl-1H-pyrazol-3-yl)cyclopropane-1-carboxylic acid (250 mg, 1.50 mmol) was dissolved in POCl₃ (2 mL), followed by the addition of Thiosemicarbazide (137 mg, 1.50 mmol). The reaction mixture was stirred at 80 °C for 2 hrs. After completion, the reaction mixture was poured into ice cold water, 10% NaOH solution was added dropwise to bring pH at ~7 and the resulting mixture was extracted with EtOAc (3×). The combined organic layers were dried over Na₂SO₄, filtered, and concentrated in vacuo to afford 5-((1R,2R)-2-(1-methyl-1H-pyrazol-3-yl)cyclopropyl)-1,3,4-thiadiazol-2-amine (150 mg, 45% yield) which was used in the next step without any further purification. LCMS m/z 221.8 [M+H]⁺.

### Step 2/Compound 3

To a solution of racemic 5-((1R,2R)-2-(1-methyl-1H-pyrazol-3-yl)cyclopropyl)-1,3,4-thiadiazol-2-amine (40 mg, 0.18 mmol) in pyridine (1 ml) was added Intermediate 2 (53 mg, 0.21 mmol) and EDC·HCl (100 mg, 0.539 mmol). The reaction mixture was stirred at rt for 2 hrs. The reaction mixture was quenched in water and extracted with EtOAc (3×). The combined organic layers were dried over Na₂SO₄, filtered, and concentrated under vacuum. The residue was purified by silica gel chromatography eluting using MeOH (8%) in DCM. Appropriate fractions were combined and concentrated to afford a racemic mixture of Compound 3 (30 mg, 36% yield). ¹H NMR (400 MHz, DMSO-d₆) δ 13.03 (s, 1H), 8.77 (d, J=4.0 Hz, 1H), 8.66 (s, 1H), 7.69-7.68 (d, J=4.2 Hz, 1H), 7.32-7.30 (t, J=8.0 Hz, 1H), 7.22-7.20 (t, J=8.0 Hz, 1H) 6.12-6.11 (d, J=4.2 Hz, 1H), 3.77 (s, 3H), 3.47 (s, 3H), 2.66-2.64 (t, J=8.0 Hz, 2H), 1.63-1.58 (m, 2H). LCMS m/z 451.0 [M+H]⁺.

### Compound 4/Method A/Racemic 3-(5-fluoro-2-methoxyphenyl)-N-(5-((1R,2R)-2-(1-methyl-1H-benzo[d]imidazol-2-yl)cyclopropyl)-1,3,4-thiadiazol-2-yl)isonicotinamide

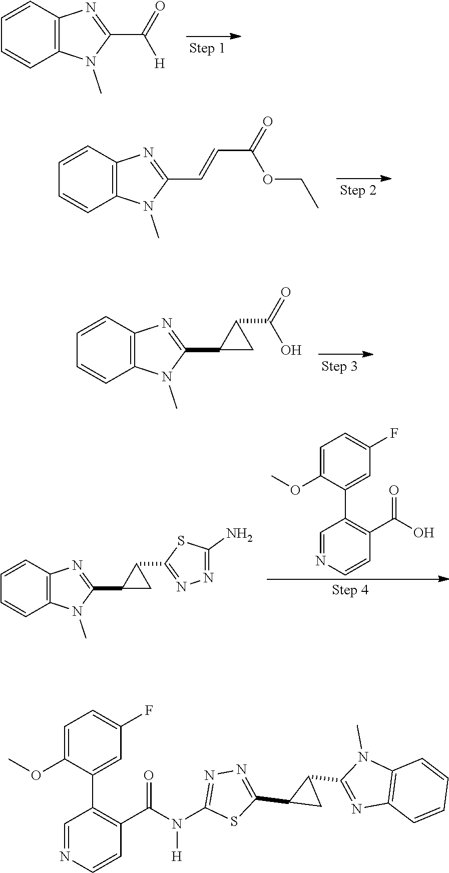 [SMILES: Cn1c(C=O)nc2ccccc21 ; CCOC(=O)/C=C/c1nc2ccccc2n1C ; Cn1c([C@@H]2C[C@H]2C(=O)O)nc2ccccc21 ; Cn1c([C@@H]2C[C@H]2c2nnc(N)s2)nc2ccccc21 ; COc1ccc(F)cc1-c1cnccc1C(=O)O ; COc1ccc(F)cc1-c1cnccc1C(=O)Nc1nnc([C@@H]2C[C@H]2c2nc3ccccc3n2C)s1]

### Step 1/Ethyl (E)-3-(1-methyl-1H-benzo[d]imidazol-2-yl)acrylate

To a solution of 1-methyl-1H-benzo[d]imidazole-2-carbaldehyde (1.00 g, 6.25 mmol) in THF (10 mL), a solution of ethyl(triphenylphosphoranylidene)acetate (3.26 g, 9.37 mmol) in THF (5 mL) was added at 0 °C and the resulting mixture was stirred for 16 hrs at rt. The resulting reaction mixture was diluted with water and extracted with EtOAc (3×). The combined organic layers were dried over Na₂SO₄ and concentrated under vacuum. The residue was purified by silica gel chromatography eluting EtOAc (40%) in hexanes. The appropriate fractions were combined and concentrated to afford ethyl (E)-3-(1-methyl-1H-benzo[d]imidazol-2-yl)acrylate (1.00 g, 69% yield). LCMS m/z 232.14 [M+H]⁺.

### Step 2/Racemic (1R,2R)-2-(1-methyl-1H-benzo[d]imidazol-2-yl)cyclopropane-1-carboxylic acid

To a suspension of NaH (60% dispersion in oil, 208 mg, 5.20 mmol) in DMSO (6 mL) at 10 °C was added trimethylsulfoxonium iodide (1.01 g, 4.50 mmol). The resulting mixture was stirred for 10 min at 10 °C followed by the addition of ethyl a solution of (E)-3-(1-methyl-1H-benzo[d]imidazol-2-yl)acrylate (600 mg, 2.6 mmol) in 6 mL of THF. The final reaction mixture was stirred at rt for 16 hrs. The reaction mixture was quenched in ice cold water and non-polar impurities were removed by extraction with EtOAc (2×). The aqueous layer was collected and neutralized to pH 7 with diluted formic acid. The product was extracted with DCM/iso-propyl amine (3/1) (3×). The combined organic layers were concentrated under vacuum to afford racemic (1R,2R)-2-(1-methyl-1H-benzo[d]imidazol-2-yl)cyclopropane-1-carboxylic acid (100 mg, 18% yield) which was used without further purification. LCMS m/z 217 [M+H]⁺.

### Step 3: Racemic 5-((1R,2R)-2-(1-methyl-1H-benzo[d]imidazol-2-yl)cyclopropyl)-1,3,4-thiadiazol-2-amine

POCl₃ (3 mL) was added to a mixture of racemic (1R,2R)-2-(1-methyl-1H-benzo[d]imidazol-2-yl)cyclopropane-1-carboxylic acid (40 mg, 0.18 mmol) and thiosemicarbazide (16 mg, 0.18 mmol). The reaction mixture was stirred for 1 hour at 80 °C. The resulting reaction mixture was poured in ice cold water and neutralized with 5% aqueous NaOH (1N). The desired compound was extracted with EtOAc (3×). The combined organic layers were concentrated under vacuum. The residue was purified by silica gel chromatography and the product was eluted using MeOH (3%) in DCM. The appropriate fractions were combined and concentrated in vacuo to afford racemic 5-((1R,2R)-2-(1-methyl-1H-benzo[d]imidazol-2-yl)cyclopropyl)-1,3,4-thiadiazol-2-amine (25 mg, 49% yield). LCMS m/z 272 [M+H]⁺.

### Step 4: Compound 4

To a mixture of racemic 5-((1R,2R)-2-(1-methyl-1H-benzo[d]imidazol-2-yl)cyclopropyl)-1,3,4-thiadiazol-2-amine (35 mg, 0.12 mmol) and Intermediate 2 (47 mg, 0.19 mmol) in pyridine (0.5 mL) was added EDC·HCl (73 mg, 0.35 mmol). The reaction mixture was stirred for 1.5 hour at rt. The resulting reaction mixture was poured in ice cold water and extracted with EtOAc (3×). The combined organic layers were concentrated under vacuum. The residue was purified by reverse phase preparative HPLC carried out using SUNFIRE C₁₈ (250×19 mm) column and eluting with a gradient of water (0.1% FA) in CH₃CN (0.1% FA) as the mobile phase. Appropriate fractions were combined and lyophilized to afford a racemic mixture of Compound 4 (5.0 mg, 8% yield). ¹H NMR (400 MHz, DMSO-d₆) δ 12.06 (s, 1H), 8.76 (d, J=5.2 Hz, 1H), 8.65 (s, 1H), 7.7 (d, J=4.8 Hz, 1H), 7.56-7.51 (m, 2H), 7.32-7.29 (m, 1H), 7.24-7.15 (m, 3H), 7.01-6.98 (m, 1H), 3.86 (s, 3H), 3.49 (s, 3H), 3.06-2.90 (m, 2H), 1.92-1.84 (m, 2H). LCMS m/z 501.4 [M+H]⁺.

### Compound 26/Method A/2′-chloro-N-(5-((1R,2R)-2-(4-cyanophenyl)cyclopropyl)-1,3,4-thiadiazol-2-yl)-5′-methoxy-[3,4′-bipyridine]-4-carboxamide

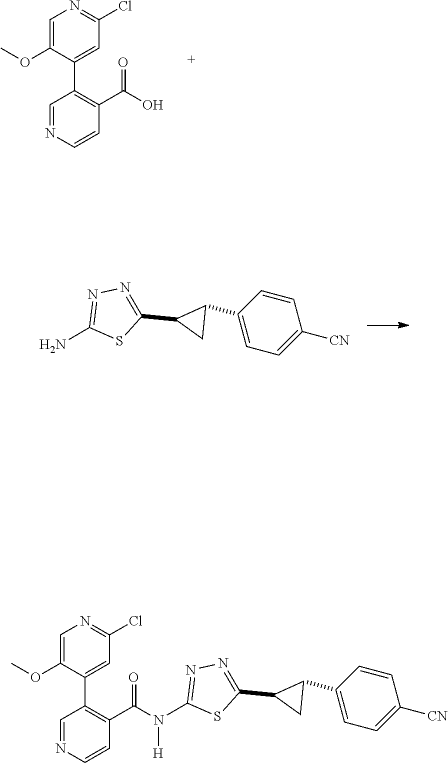 [SMILES: COc1cnc(Cl)cc1-c1cnccc1C(=O)O ; N#Cc1ccc([C@@H]2C[C@H]2c2nnc(N)s2)cc1 ; COc1cnc(Cl)cc1-c1cnccc1C(=O)Nc1nnc([C@@H]2C[C@H]2c2ccc(C#N)cc2)s1]

To a solution of Intermediate 15 (20.0 mg, 75.6 μmol) and Intermediate 16 (19.3 mg, 79.7 μmol) in pyridine (0.4 mL) was added EDC·HCl (29.7 mg, 155 μmol). The reaction was stirred at 50 °C for 30 min. The crude reaction mixture was filtered, and the filtrate was purified by preparative HPLC (Phenomenex Gemini®) eluting with a gradient of CH₃CN (40 to 70%) in water both containing 0.1% formic acid. Appropriate fractions were combined and lyophilized to afford Compound 26 (10.0 mg, 26% yield). ¹HNMR (400 MHz, DMSO-d₆) δ 13.16 (s, 1H), 8.79 (d, J=5.0 Hz, 1H), 8.67 (s, 1H), 8.10 (s, 1H), 7.75-7.70 (m, 3H), 7.56 (s, 1H), 7.42-7.37 (m, 2H), 3.54 (s, 3H), 2.84 (dt, J=9.4, 5.3 Hz, 1H), 2.68 (dt, J=9.2, 5.9 Hz, 1H), 1.77 (dt, J=9.0, 5.3 Hz, 1H), 1.69 (dt, J=8.7, 5.4 Hz, 1H). LCMS m/z 489.1 [M+H]⁺.

### Compound 6/Method B/3-(5-cyano-2-methoxyphenyl)-N-(5-(spiro[2.2]pentan-1-ylethynyl)-1,3,4-thiadiazol-2-yl)isonicotinamide

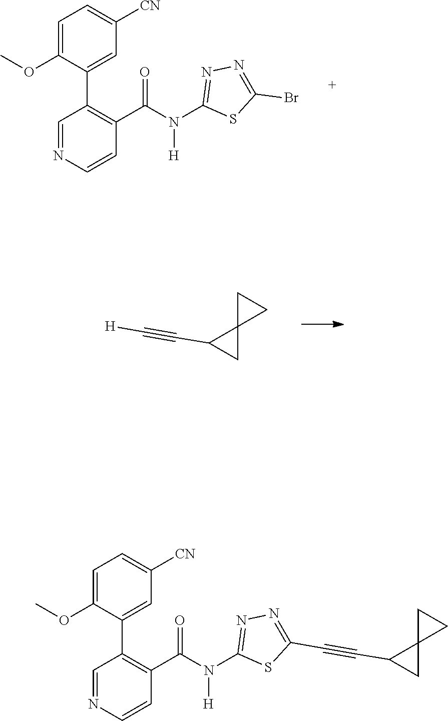 [SMILES: COc1ccc(C#N)cc1-c1cnccc1C(=O)Nc1nnc(Br)s1 ; C#CC1CC12CC2 ; COc1ccc(C#N)cc1-c1cnccc1C(=O)Nc1nnc(C#CC2CC23CC3)s1]

N₂ was bubbled through a solution of Intermediate 12 (81 mg, 195 μmol) 2-ethynylspiro[2.2]pentane (57.2 mg, 621 μmol) triethylamine (220 UL, 1.58 mmol) in dry DMF (1 mL) while sonicating for 15 min. Pd(PPh₃)₄ (45.7 mg, 39.5 μmol). The reaction mixture was stirred at 50 °C overnight. The crude reaction mixture was filtered, and the filtrate was purified by preparative HPLC (Phenomenex Gemini®) eluting with a gradient of CH₃CN (50 to 80%) in water both containing 0.1% formic acid. Appropriate fractions were combined and lyophilized to afford Compound 6 (21.3 mg, 26% yield). ¹H NMR (400 MHz, DMSO-d₆) δ 13.40 (s, 1H), 8.76 (dd, J=5.0, 0.9 Hz, 1H), 8.66 (s, 1H), 7.89-7.83 (m, 2H), 7.69 (d, J=5.0 Hz, 1H), 7.11 (d, J=8.5 Hz, 1H), 3.49 (s, 3H), 2.08 (dd, J=7.8, 4.4 Hz, 1H), 1.50 (dd, J=7.9, 3.9 Hz, 1H), 1.30 (t. J=4.2 Hz, 1H), 1.02-0.96 (m, 1H), 0.96-0.84 (m, 3H). LCMS m/z 428.0 [M+H]⁺.

### Compound 7/Method B: 3-(5-cyano-2-methoxyphenyl)-N-(5-(oxetan-3-ylethynyl)-1,3,4-thiadiazol-2-yl)isonicotinamide

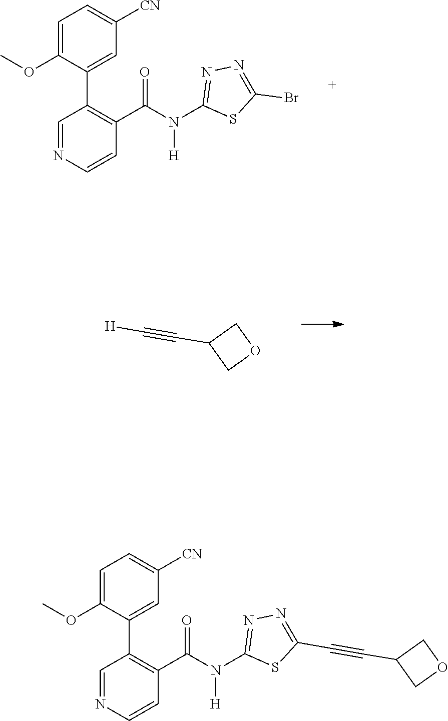 [SMILES: COc1ccc(C#N)cc1-c1cnccc1C(=O)Nc1nnc(Br)s1 ; C#CC1COC1 ; COc1ccc(C#N)cc1-c1cnccc1C(=O)Nc1nnc(C#CC2COC2)s1]

N₂ was bubbled through a solution of Intermediate 12 (83.0 mg, 199 μmol), 3-ethynyloxetane (52.3 mg, 637 μmol), triethylamine (225 μL, 1.61 mmol) in dry DMF (1 mL) while sonicating for 15 min. Pd(PPh₃)₄ (46.8 mg, 40.5 μmol) was then added and the reaction mixture was stirred at 50 °C overnight. The crude reaction mixture was filtered, and the filtrate was purified by preparative HPLC (Phenomenex Gemini®) eluting with a gradient of CH₃CN (30 to 60%) in water both containing 0.1% formic acid. Appropriate fractions were combined and lyophilized to afford Compound 7 (39.1 mg, 47% yield). ¹H NMR (400 MHz, DMSO-d₆) δ 13.48 (s, 1H), 8.77 (d, J=5.0 Hz, 1H), 8.67 (s, 1H), 7.90-7.83 (m, 2H), 7.73-7.69 (m, 1H), 7.12 (d, J=8.6 Hz, 1H), 4.79 (dd, J=8.5, 5.6 Hz, 2H), 4.61 (dd, J=6.9, 5.5 Hz, 2H), 4.24 (tt, J=8.6, 7.0 Hz, 1H), 3.49 (s, 3H). LCMS m/z 418.1 [M+H]⁺.

### Compound 8/Method C/N-(5-((1-(cyanomethyl)-1H-pyrazol-4-yl)ethynyl)-1,3,4-thiadiazol-2-yl)-3-(5-fluoro-2-methoxyphenyl)isonicotinamide

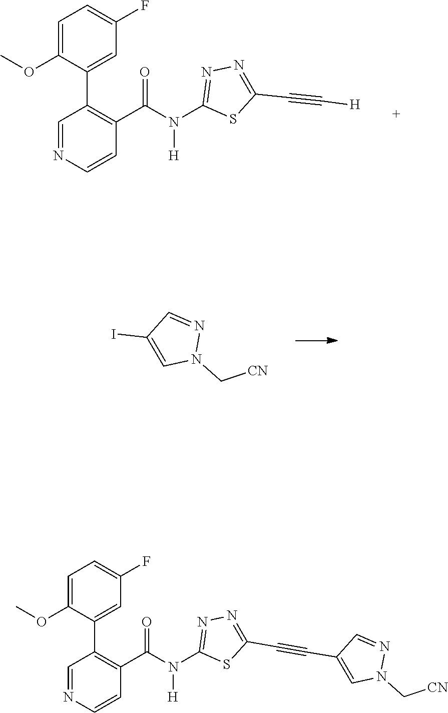 [SMILES: C#Cc1nnc(NC(=O)c2ccncc2-c2cc(F)ccc2OC)s1 ; N#CCn1cc(I)cn1 ; COc1ccc(F)cc1-c1cnccc1C(=O)Nc1nnc(C#Cc2cnn(CC#N)c2)s1]

N₂ was bubbled through a solution of Intermediate 11 (83.0 mg, 234 μmol) 2-(4-iodopyrazol-1-yl)acetonitrile (164 mg, 703 μmol), Triethylamine (264 μL, 1.89 mmol) in dry DMF (0.25 mL) while sonicating for 15 min. Pd(PPh₃)₄ (54.1 mg, 46.9 μmol) was added and the resulting mixture was stirred at 80 °C overnight. The crude reaction mixture was filtered, and the filtrate was purified by preparative HPLC (Phenomenex Gemini®) eluting with a gradient of CH₃CN (35 to 65%) in water both containing 0.1% formic acid. Appropriate fractions were combined and lyophilized to afford Compound 8 (23.2 mg, 22% yield) ¹H NMR (400 MHz, DMSO-d₆) δ 13.42 (s, 1H), 8.69 (d, J=5.0 Hz, 1H), 8.57 (s, 1H), 8.35 (s, 1H), 7.97 (s, 1H), 7.66 (d, J=5.0 Hz, 1H), 7.40-7.34 (m, 1H), 6.90-6.82 (m, 2H), 5.52 (s, 2H), 3.44 (s, 3H). LCMS m/z 460.2 [M+H]⁺.

### Compound 12/Method D/3-(5-cyano-2-methoxyphenyl)-N-(5-(cyclopropylethynyl)-1,3,4-thiadiazol-2-yl)isonicotinamide

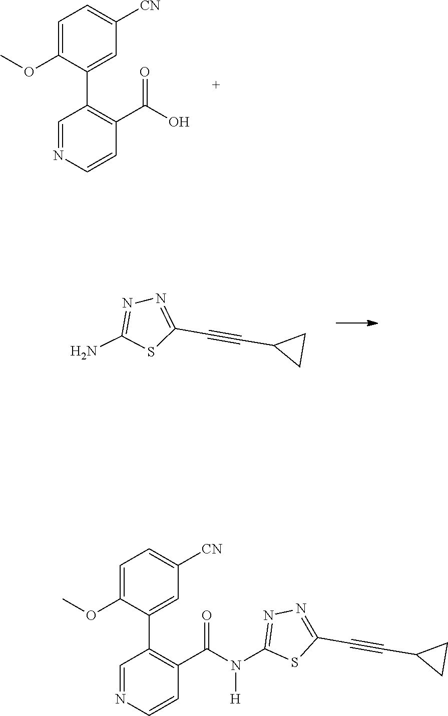 [SMILES: COc1ccc(C#N)cc1-c1cnccc1C(=O)O ; Nc1nnc(C#CC2CC2)s1 ; COc1ccc(C#N)cc1-c1cnccc1C(=O)Nc1nnc(C#CC2CC2)s1]

To a solution of Intermediate 3 (54.0 mg, 212 μmol) and Intermediate 9 (35.0 mg, 212 μmol) in pyridine (0.5 mL) was added EDC·HCl (63 mg, 326 μmol). The reaction was stirred at 25 °C for 2 hrs. The crude reaction mixture was filtered, and the filtrate was purified by preparative HPLC (Phenomenex Gemini®) eluting with a gradient of CH₃CN (35 to 65%) in water both containing 0.1% formic acid. Appropriate fractions were combined and lyophilized to afford Compound 12 (22.8 mg, 33% yield). ¹H NMR (400 MHz, DMSO-d₆) δ 13.42 (s, 1H), 8.80 (d, J=5.0 Hz, 1H), 8.69 (s, 1H), 7.92-7.87 (m, 2H), 7.73 (dd, J=5.0, 0.7 Hz, 1H), 7.15 (d, J=8.5 Hz, 1H), 3.52 (s, 3H), 1.70 (tt, J=8.3, 5.0 Hz, 1H), 1.05-0.94 (m, 2H), 0.91-0.81 (m, 2H). LCMS m/z 402.2 [M+H]⁺.

### Compound 27/Method D/3-[2-methoxy-5-(trifluoromethyl)phenyl]-N-[5-[2-(5-methyl-1H-pyrazol-3-yl)ethynyl]-1,3,4-thiadiazol-2-y]pyridine-4-carboxamide

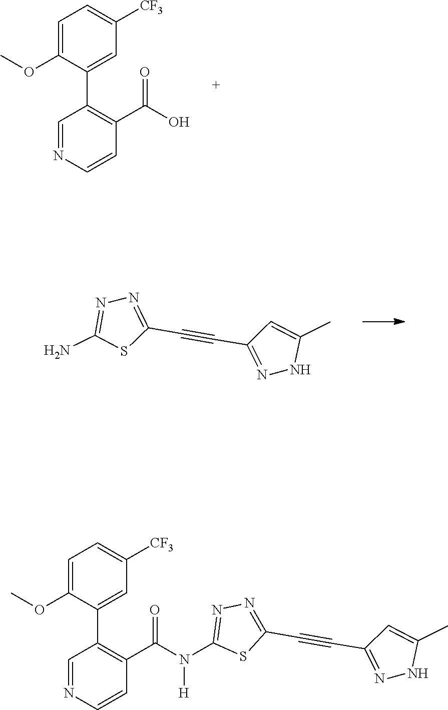

To a solution of Intermediate 14 (50.0 mg, 168 μmol) and Intermediate 17 (38.3 mg, 186 μmol) in Pyridine (0.4 mL) was added EDC·HCl (66.5 mg, 347 μmol) in one shot. The reaction was stirred at 50 °C for 30 min. The crude reaction mixture was filtered, and the filtrate was purified by preparative HPLC (Phenomenex Gemini®) eluting with a gradient of CH₃CN (40 to 70%) in water both containing 0.1% formic acid. Appropriate fractions were combined and lyophilized to afford Compound 27 (16 mg, 19% yield). ¹H NMR (400 MHz, DMSO-d₆) δ 13.53 (s, 1H), 13.26-12.98 (m, 1H), 8.80-8.74 (m, 1H), 8.68 (d, J=2.1 Hz, 1H), 7.71 (dd, J=5.1, 2.3 Hz, 3H), 7.14 (d, J=8.4 Hz, 1H), 6.40 (s, 1H), 3.51 (d, J=2.1 Hz, 3H), 2.22 (d, J=2.1 Hz, 3H). LCMS m/z 485.1 [M+H]⁺.

### Compound 28/Method D/3-(2-chloro-5-methoxy-4-pyridyl)-N-[5-[2-(5-methyl-1H-pyrazol-3-yl)ethynyl]-1,3,4-thiadiazol-2-yl]pyridine-4-carboxamide

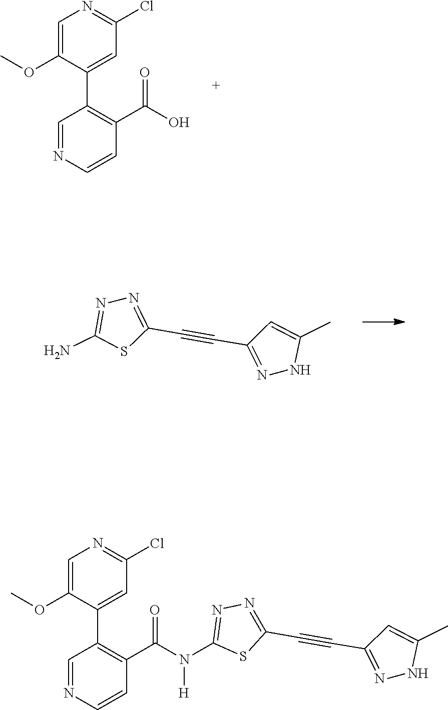

To a solution of Intermediate 15 (25.0 mg, 94.5 μmol) and Intermediate 17 (20.5 mg, 99.9 μmol) in Pyridine (0.4 mL) was added EDC·HCl (37.4 mg, 195 μmol) in one shot. The reaction was stirred at 50 °C for 30 min. The crude reaction mixture was filtered, and the filtrate was purified by preparative HPLC (Phenomenex Gemini®) eluting with a gradient of CH₃CN (35 to 65%) in water both containing 0.1% formic acid. Appropriate fractions were combined and lyophilized to afford Compound 28 (7.6 mg, 17% yield). ¹H NMR (400 MHz, DMSO-d₆) δ 13.64 (s, 1H), 13.13 (s, 1H), 8.83 (d, J=5.0 Hz, 1H), 8.71 (s, 1H), 8.10 (s, 1H), 7.77 (d, J=5.0 Hz, 1H), 7.61 (s, 1H), 6.39 (s, 1H), 3.53 (s, 3H), 2.22 (s, 3H). LCMS m/z 452.1 [M+H]⁺.

### Compound 31/Method D/N-(5-(cyclopropylethynyl)-1,3,4-thiadiazol-2-yl)-2′-(difluoromethyl)-5′-methoxy-[3,4′-bipyridine]-4-carboxamide

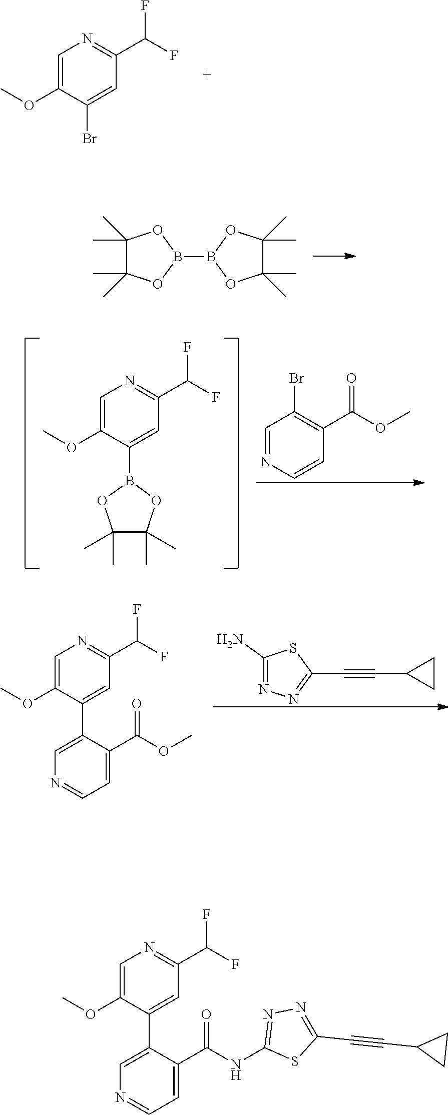

### Step 1/methyl 2′-(difluoromethyl)-5′-methoxy-[3,4′-bipyridine]-4-carboxylate

4-bromo-2-(difluoromethyl)-5-ethoxypyridine (500 mg, 2.10 mmol), 4,4,4′,4′,5,5,5′,5′-octamethyl-2,2′-bi(1,3,2-dioxaborolane) (825 mg, 3.15 mmol) and KOAc (521 mg, 5.25 mmol) were combined in 1,4-dioxane (10 mL). N₂ was bubbled in the mixture for 2 min then PdCl₂(dppf) (157 mg, 0.21 mmol) was added. N₂ bubbling was pursued for 5 minutes, then the vial was sealed and heated to 65 °C for 18 hrs. The reaction mixture was cooled to rt, filtered through a plug of silica gel, washed with EtOAc and the filtrate was concentrated in vacuo to afford the desired pinacol boronate as pale brown oil, which was carried forward in the next step without purification. The later was dissolved in 1,4-dioxane (10 mL). Methyl 3-bromoisonicotinate (463 mg, 2.10 mmol), K₂CO₃ (726 mg, 5.25 mmol) and water (2 mL) were added. N₂ was bubbled in the mixture for 2 min and PdCl₂(dppf) (157 mg, 0.21 mmol) was added. N₂ bubbling was pursued for 5 minutes, the vial was sealed, and the final reaction mixture was stirred at 80 °C for 4 hrs. The resulting mixture was cooled to rt, filtered through a plug of silica gel eluting with EtOAc. The filtrate was adsorbed onto silica gel. Purification by silica gel chromatography eluting with EtOAc (2%-100%) in Heptane. Appropriate fractions were combined and concentrated in vacuo to afford methyl 2′-(difluoromethyl)-5′-methoxy-[3,4′-bipyridine]-4-carboxylate (385 mg, 62%). LCMS m/z 295.0 [M+H]⁺.

### Step 2/Compound 31

Methyl 2′-(difluoromethyl)-5′-methoxy-[3,4′-bipyridine]-4-carboxylate (385 mg, 1.31 mmol) and Intermediate 9 (238 mg, 1.44 mmol) were dissolved in THF (15 mL). 1,5,7-triazabicyclo[4.4.0]dec-5-ene (939 mg, 6.54 mmol) was added, and the resulting mixture was stirred at 90 °C for 2 hrs. The reaction mixture was cooled to rt, diluted with EtOAc, and washed with saturated aqueous NH₄Cl. The organic layer was dried over Na₂SO₄, filtered, and concentrated in vacuo. The residue was purified by reverse phase chromatography eluting with CH₃CN (10% to 100%) in 10 mM Ammonium Formate buffer. The appropriate fractions were combined and lyophilized to afford Compound 31 (283 mg, 51% yield). ¹H NMR (400 MHz, DMSO-d₆) δ 13.52 (s, 1H), 8.83 (d, J=5.0 Hz, 1H), 8.72 (s, 1H), 8.41 (s, 1H), 7.77 (d, J=5.0 Hz, 1H), 7.73 (s, 1H), 6.95 (t, J=55.0 Hz, 1H), 3.61 (s, 3H), 1.67 (tt. J=8.3, 5.0 Hz, 1H), 1.01-0.92 (m, 2H), 0.89-0.78 (m, 2H). LCMS m/z 428.2 [M+H]⁺.

### Compound 32/Method D/2′-chloro-N-(5-(cyclopropylethynyl)-1,3,4-thiadiazol-2-yl)-5′-methoxy-1-methyl-6-oxo-1,6-dihydro-[3,4′-bipyridine]-4-carboxamide

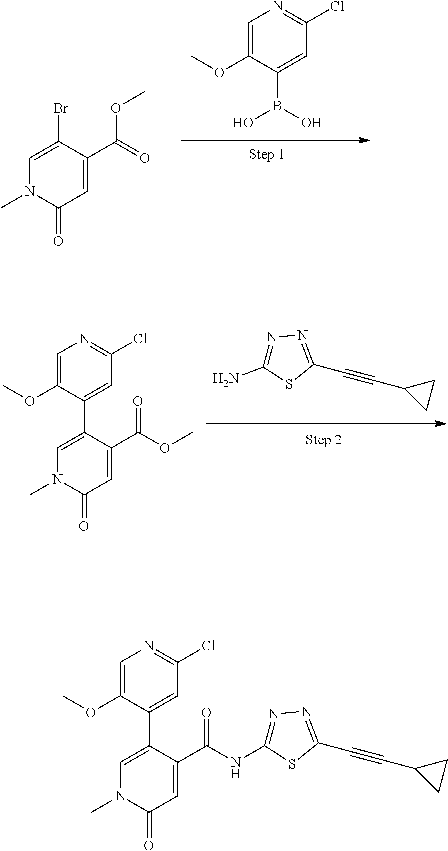

### Step 1/methyl 2′-chloro-5′-methoxy-1-methyl-6-oxo-1,6-dihydro-[3,4′-bipyridine]-4-carboxylate

In a 10 mL seal tube, methyl 5-bromo-1-methyl-2-oxo-1,2-dihydropyridine-4-carboxylate (0.100 g, 0.406 mmol) and (2-chloro-5-methoxypyridin-4-yl)boronic acid (0.098 g, 0.52 mmol) were dissolved in 1,4-dioxane (2 mL) followed by addition of Cs₂CO₃ (0.32 g, 1.01 mmol) and water (0.2 mL). The reaction mixture was purged with N₂ gas for 15 min and PdCl₂(dppf) (0.033 g, 0.0406 mmol) was added. The reaction mixture was stirred at 90 °C for 2 h. The reaction mixture was quenched in water (10 mL) and extracted with EtOAc (3×10 mL). The combined organic layers were dried over Na₂SO₄, filtered, and concentrated under vacuum. The residue was purified by silica gel flash chromatography eluting EtOAc (60%) in Hexane. Appropriate fractions were combined and concentrated to afford methyl 2′-chloro-5′-methoxy-1 methyl-6-oxo-1,6-dihydro-[3,4′-bipyridine]-4-carboxylate (70.0 mg, 56%) LCMS m/z 309.1 [M+H]⁺.

### Step 2/Compound 32

Methyl 2′-chloro-5′-methoxy-1-methyl-6-oxo-1,6-dihydro-[3,4′-bipyridine]-4-carboxylate (70.0 mg, 0.227 mmol) and Intermediate 9 (0.041 g, 0.249 mmol) were combined in THF (1 mL). 1,5,7-triazabicyclo[4.4.0]dec-5-ene (0.157 g, 1.13 mmol) was added and the resulting mixture was stirred at 70 °C temperature for 1 h. The reaction mixture was cooled to rt, quenched in water (10 mL) and extracted with EtOAc (3×10 mL). The combined organic layers were dried over Na₂SO₄, filtered, and concentrated under vacuum. The residue was purified by silica gel flash chromatography eluting with MeOH (5%) in DCM. Appropriate fractions were combined and concentrated and further purified by reverse phase HPLC eluting with CH₃CN (10 to 100%) in water both containing 0.1% formic acid. Appropriate fractions were combined and lyophilized to afford Compound 32 (12.0 mg, 12%). ¹H NMR (400 MHz, DMSO-d₆) δ 13.49 (s, 1H), 8.05-8.03 (m, 2H), 7.52 (s, 1H), 6.82 (s, 1H), 3.53 (s, 3H), 3.50 (s, 3H), 1.71 (bs, 1H), 1.02 (s, 2H), 0.89 (s, 2H). LCMS m/z 442.1 [M+H]⁺.

### Compound 33/Method D/N-(5-(cyclopropylethynyl)-1,3,4-thiadiazol-2-yl)-6-(2-(dimethylamino)-2-oxoethyl)-4-(2-methoxy-5-(trifluoromethyl)phenyl)nicotinamide

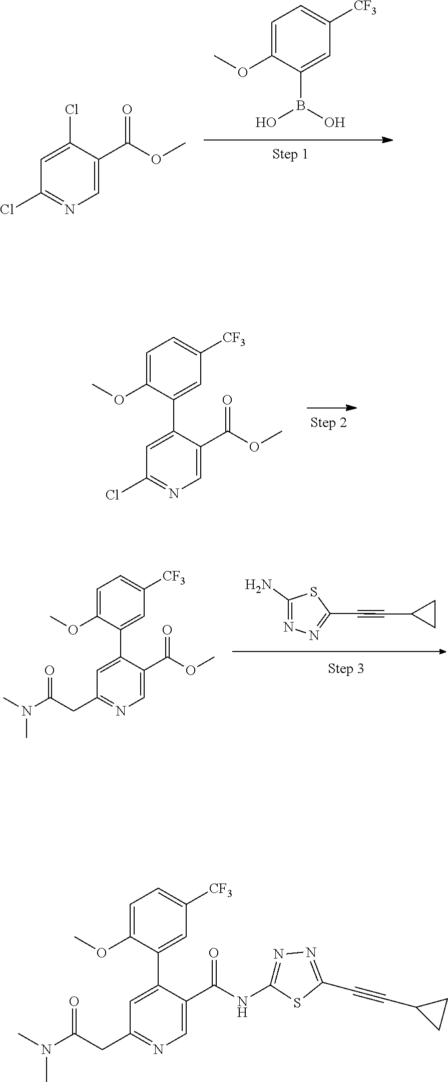

### Step 1/methyl 6-chloro-4-(2-methoxy-5-(trifluoromethyl)phenyl)nicotinate

N₂ was bubbled for 30 min in a mixture of methyl 4,6-dichloropyridine-3-carboxylate 1 (600 mg, 2.91 mmol), (2-methoxy-5-trifluoromethyl)phenyl)boronic acid (641 mg, 2.91 mmol) and K₂CO₃ (805 mg, 5.83 mmol) in H₂O (2.0 mL) and 1-4-dioxane (6.0 mL). To the resulting mixture was added PdCl₂(dtbpf) (193 mg, 296 μmol) and the resulting mixture was stirred at 80 °C overnight. The reaction mixture was cooled to rt, poured in water and extracted with EtOAc (3×). The combined organic layers were washed with brine, dried over Na₂SO₄, filtered, and concentrated in vacuo. The residue was purified by silica gel flash chromatography eluting with a gradient of EtOAc (5 to 30%) in Heptane. Appropriate fractions were combined and concentrated in vacuo to afford methyl methyl 6-chloro-4-(2-methoxy-5-(trifluoromethyl)phenyl)nicotinate (350 mg, 35% yield) as a yellow oil. LCMS m/z 346.1 [M+H]⁺.

### Step 2/methyl 6-(2-(dimethylamino)-2-oxoethyl)-4-(2-methoxy-5-(trifluoromethyl)phenyl)nicotinate

To NaHMDS solution (1 M in THF, 890 μL, 890 μmol) at −30 °C, in a sealable tube, was added dropwise N,N-dimethylacetamide (80 μL, 863 μmol). The reaction mixture was stirred at −30 °C for 1 h then ZnCl₂ solution (0.5 M in THF, 1.8 mL, 0.90 mmol) was added dropwise and the reaction mixture was warmed to rt and stirred for 3 hrs. To the resulting white suspension was added methyl 6-chloro-4-[2-methoxy-5-(trifluoromethyl)phenyl]pyridine-3-carboxylate (100 mg, 289 μmol). N₂ was bubbled through the reaction mixture while sonicating for 15 min before adding Pd(PPh₃)₄ (67 mg, 58 μmol) and sealing the tube. The reaction mixture was heated at 90 °C for 48 hrs. The reaction mixture was cooled to rt, and silica gel was added before dry-packing. The residue was purified by silica gel flash chromatography eluting with a gradient of MeOH (0 to 10%) in DCM. Appropriate fractions were combined and concentrated in vacuo to afford methyl 6-(2-(dimethylamino)-2-oxoethyl)-4-(2-methoxy-5-(trifluoromethyl)phenyl)nicotinate (140 mg, 85% yield, 70% purity) as a green oil. LCMS m/z 397.2 [M+H]⁺.

### Step 3/Compound 33

To a solution of methyl 6-(2-(dimethylamino)-2-oxoethyl)-4-(2-methoxy-5-(trifluoromethyl)phenyl)nicotinate (75.0 mg, 189 μmol) and Intermediate 9 (35.0 mg, 210 μmol) in THF (2.0 mL) was added 1,5,7-triazabicyclo[4.4.0]dec-5-ene (132 mg, 950 μmol). The reaction was stirred at 90 °C overnight. The crude reaction mixture was filtered, and the filtrate was purified by preparative HPLC (Phenomenex Gemini®) eluting with a gradient of CH₃CN (35 to 65%) in water both containing 0.1% formic acid. Appropriate fractions were combined and lyophilized to afford Compound 33 (15.9 mg, 16% yield). ¹H NMR (400 MHz, DMSO-d₆) δ 13.33 (s, 1H), 8.79 (s, 1H), 7.81-7.76 (m, 1H), 7.65 (d, J=2.4 Hz, 1H), 7.43 (s, 1H), 7.17 (d, J=8.7 Hz, 1H), 4.00 (s, 2H), 3.53 (s, 3H), 3.09 (s, 3H), 2.85 (s, 3H), 1.69 (tt, J=8.3, 5.0 Hz, 1H), 1.02-0.96 (m, 2H), 0.89-0.83 (m, 2H). LCMS m/z 530.2 [M+H]⁺.

### Compound 34/Method D/5-(2-chloro-5-(trifluoromethyl)phenyl)-N-(5-(cyclopropylethynyl)-1,3,4-thiadiazol-2-yl)-1-(2-(dimethylamino)-2-oxoethyl)-2-oxo-1,2-dihydropyridine-4-carboxamide

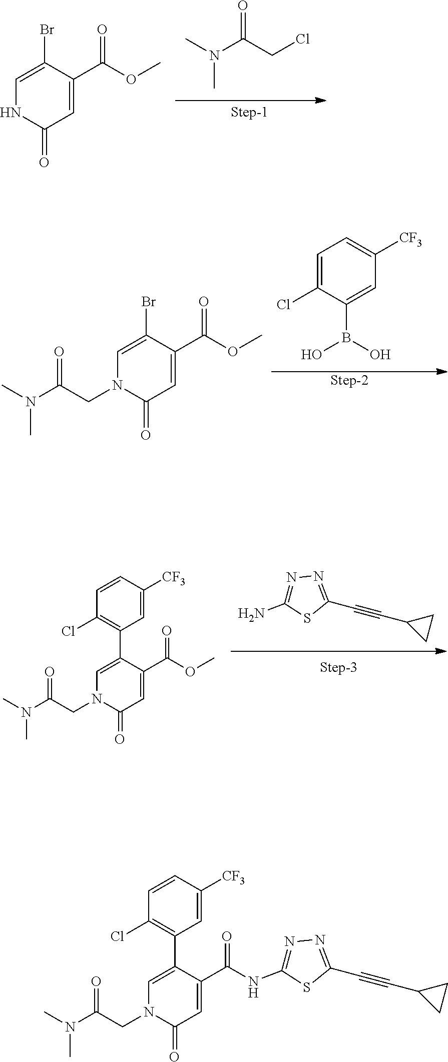

### Step 1/methyl 5-bromo-1-(2-(dimethylamino)-2-oxoethyl)-2-oxo-1,2-dihydropyridine-4-carboxylate

To methyl 5-bromo-2-oxo-1,2-dihydropyridine-4-carboxylate (1.00 g, 4.31 mmol) in ACN (10 mL) was added Cs₂CO₃ (3.50 g, 10.8 mmol) and 2-chloro-N,N-dimethylacetamide (0.786 g, 6.46 mmol). The reaction mixture was stirred at rt for 2 hrs. The reaction mixture was poured in water (30 mL) and extracted with EtOAc (3×25 mL). The combined organic layers were concentrated, and the residue was triturated with pentane, filtered and dried under vacuum to afford methyl 5-bromo-1-(2-(dimethylamino)-2-oxoethyl)-2-oxo-1,2-dihydropyridine-4-carboxylate (0.30 g, 22%). LCMS m/z 317.0 [M+H]⁺.

### Step 2/methyl 5-(2-chloro-5-(trifluoromethyl)phenyl)-1-(2-(dimethylamino)-2-oxoethyl)-2-oxo-1,2-dihydropyridine-4-carboxylate

To methyl 5-bromo-1-(2-(dimethylamino)-2-oxoethyl)-2-oxo-1,2-dihydropyridine-4-carboxylate (0.200 g, 0.630 mmol) and (2-chloro-5-(trifluoromethyl)phenyl)boronic acid (0.183 g, 0.820 mmol) in 1,4-dioxane (2 mL) was added Cs₂CO₃ (0.511 g, 1.57 mmol) and water (0.3 mL). The reaction mixture was purged with N₂ for 15 min and then PdCl₂(dpf) (51 mg, 0.43 mmol) was added. The reaction mixture was purged with N₂ gas for 10 min then stirred at 90 °C for 3 hrs. The resulting mixture was cooled to rt, quenched in water (10 mL) and extracted with EtOAc (3×10 mL), then combined organic layer was dried over Na₂SO₄, filtered and concentrated. The residue was purified by silica gel flash chromatography eluting with EtOAc (60%) in Hexanes. Appropriate fractions were combined and concentrated to afford methyl 5-(2-chloro-5-(trifluoromethyl)phenyl)-1-(2-(dimethylamino)-2-oxoethyl)-2-oxo-1,2-dihydropyridine-4-carboxylate (70 mg, 27% yield). LCMS: m/z 417.1 [M+H]⁺.

### Step 3/Compound 34

To methyl 5-(2-chloro-5-(trifluoromethyl)phenyl)-1-(2-(dimethylamino)-2-oxoethyl)-2-oxo-1,2-dihydropyridine-4-carboxylate (80.0 mg, 0.192 mmol) and Intermediate 9 (30.0 mg, 0.192 mmol) in THF (0.5 mL) was added 1,5,7-triazabicyclo[4.4.0]dec-5-ene (132 mg, 0.96 mmol). The reaction mixture was stirred at 70 °C for 1 h. The resulting mixture was cooled to rt, quenched in water (10 mL) and extracted with EtOAc (3×10 mL). The combined organic layers were dried over Na₂SO₄, filtered, and concentrated under vacuum. The residue was purified by preparative HPLC eluting with CH₃CN (10 to 100%) in water both containing 0.1% formic acid. Appropriate fractions were combined and lyophilized to afford Compound 34 (12.0 mg, 11%). ¹H NMR (400 MHz, DMSO-d₆) δ 13.62 (s, 1H), 7.82 (s, 1H), 7.76-7.70 (m, 3H), 6.93 (s, 1H), 4.89 (s, 2H), 3.06 (s, 3H), 2.87 (s, 3H), 1.69 (m, 1H), 0.99-0.98 (m, 3H), 0.86 (s, 3H). LCMS m/z 550.2 [M+H]⁺.

### Compound 35/Method D/2-(2-chloro-5-methoxypyridin-4-yl)-N-(5-(cyclopropylethynyl)-1,3,4-thiadiazol-2-yl)-4-(2-(dimethylamino)-2-oxoethyl)benzamide

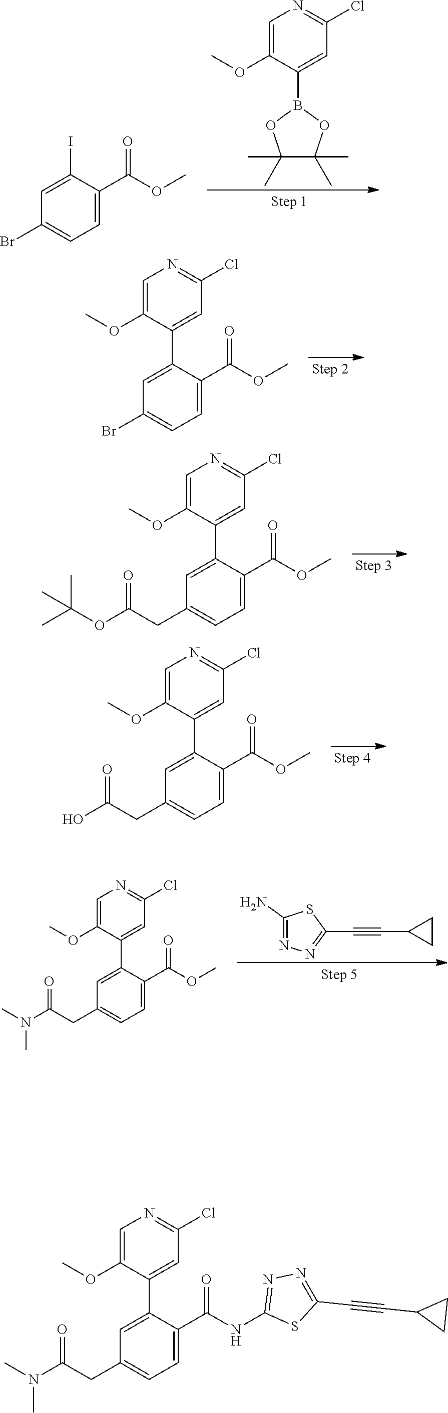

### Step 1/methyl 4-bromo-2-(2-chloro-5-methoxypyridin-4-yl)benzoate

To a solution of methyl 4-bromo-2-iodo-benzoate (1.00 g, 2.93 mmol) in dioxane (10 mL) were added Na₂CO₃ (2 M, 3.00 mL, 6 mmol), 2-chloro-5-methoxy-4-(4,4,5,5-tetramethyl-1,3,2-dioxaborolan-2-yl)pyridine (800 mg, 2.97 mmol) and PdCl₂(dppf) (210 mg, 287 μmol) in dioxane (10 mL). The mixture was degassed in vacuo, backfilled with N₂ The mixture was stirred at 60 °C for 10 hrs. The volatiles were removed in vacuo. The residue was purified on silica gel chromatography eluting with EtOAc (0-40%) in heptane. Appropriate fractions were combined and concentrated to afford methyl 4-bromo-2-(2-chloro-5-methoxypyridin-4-yl)benzoate (650 mg, 62% yield) as a white solid. LCMS m/z 356.2 [M+H]⁺.

### Step 2/methyl 4-(2-(tert-butoxy)-2-oxoethyl)-2-(2-chloro-5-methoxypyridin-4-yl)benzoate

To an oven-dried flask were added methyl 4-bromo-2-(2-chloro-5-methoxy-4-pyridyl)benzoate (320 mg, 897 μmol), (2-tert-butoxy-2-oxo-ethyl)zinc chloride (0.5 M, 7.00 mL, 3.50 mmol) and Pd(t-Bu₃P)₂ (46.0 mg, 90.0 μmol) in THF (5 mL). The reaction mixture was flushed with N₂ for 5 min and then the mixture was stirred at 70 °C for 10 hrs. After cooling to rt, the reaction was quenched with sat. NH₄Cl, diluted with water, extracted with EtOAc (3×20 mL). The combined organic extracts were washed with brine, dried over Na₂SO₄, filtered, and concentrated to dryness. The residue was purified on silica gel chromatography eluting with EtOAc (0 to 50%) in Heptane. Appropriate fractions were combined and concentrated to afford methyl 4-(2-(tert-butoxy)-2-oxoethyl)-2-(2-chloro-5-methoxypyridin-4-yl)benzoate (20 mg, 6% yield) as a white solid.

### Step 3/2-(3-(2-chloro-5-methoxypyridin-4-yl)-4-(methoxycarbonyl)phenyl)acetic acid

To a solution of methyl 4-(2-(tert-butoxy)-2-oxoethyl)-2-(2-chloro-5-methoxypyridin-4-yl)benzoate (20 mg, 51.04 μmol) in DCM (0.5 mL) was added TFA (500 μL, 6.49 mmol). The mixture was stirred at rt for 1 h. The volatiles were removed in vacuo to provide 2-[3-(2-chloro-5-methoxy-4-pyridyl)-4-methoxycarbonyl-phenyl]acetic acid (22 mg) as an off-white solid which was used directly in next step without purification. LCMS m/z 336.2 [M+H]⁺.

### Step 4/methyl 2-(2-chloro-5-methoxypyridin-4-yl)-4-(2-(dimethylamino)-2-oxoethyl)benzoate

To a solution of crude 2-(3-(2-chloro-5-methoxypyridin-4-yl)-4-(methoxycarbonyl)phenyl)acetic acid (22 mg, 49 μmol) in DMF (1 mL) were added HATU (30.0 mg, 78.9 μmol) and Dimethylamine (2 M in THF, 150 μL, 300 μmol). The mixture was stirred at rt for 0.5 h. The volatiles were removed in vacuo. The residue was purified on silica gel chromatography eluting with EtOAc (0-100%) in Hep to provide methyl 2-(2-chloro-5-methoxypyridin-4-yl)-4-(2-(dimethylamino)-2-oxoethyl)benzoate (17 mg, 95% yield) as a white solid. LCMS m/z 363.3 [M+H]⁺.

### Step 5/Compound 35

To a solution of methyl 2-(2-chloro-5-methoxypyridin-4-yl)-4-(2-(dimethylamino)-2-oxoethyl)benzoate (17.0 mg, 47 μmol) and Intermediate 9 (10.0 mg, 61 μmol) in THF (2 mL) was added 1,5,7-triazabicyclo[4.4.0]dec-5-ene (20.0 mg, 144 μmol). The reaction was stirred at 70 °C for 16 hrs. The volatiles were removed in vacuo. The residue was dissolved in DMSO, filtered, and the filtrate was purified by preparative HPLC eluting with a gradient of CH₃CN (30 to 100%) in water both containing 0.1% formic acid. Appropriate fractions were combined and lyophilized to afford Compound 35 (10 mg, 43% yield). ¹H NMR (400 MHz, DMSO-d₆) δ 13.16 (s, 1H), 8.03 (s, 1H), 7.71 (d, J=7.9 Hz, 1H), 7.46-7.30 (m, 2H), 7.29 (s, 1H), 3.78 (s, 2H), 3.49 (s, 3H), 3.01 (s, 3H), 2.80 (s, 3H), 1.65 (m, 1H), 0.94 (m, 2H), 0.88-0.75 (m, 2H). LCMS m/z 496.6 [M+H]⁺.

### Compound 36/Method D/N-(5-(cyclopropylethynyl)-1,3,4-thiadiazol-2-yl)-6-(2-(difluoromethyl)-5-methoxypyridin-4-yl)-[1,2,4]triazolo[1,5-a]pyridine-7-carboxamide

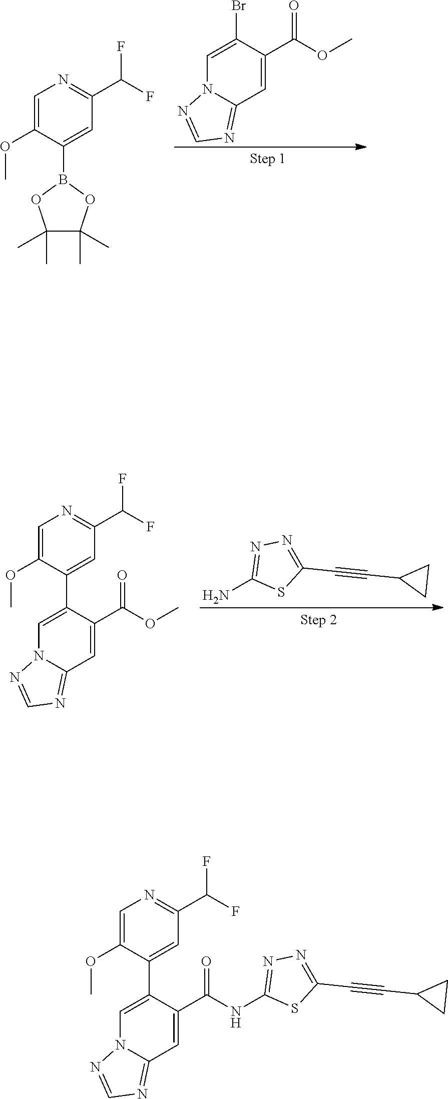

### Step 1/methyl 6-(2-(difluoromethyl)-5-methoxypyridin-4-yl)-[1,2,4]triazolo[1,5-a]pyridine-7-carboxylate

In a 10 mL sealed tube, 2-(difluoromethyl)-5-methoxy-4-(4,4,5,5-tetramethyl-1,3,2-dioxaborolan-2-yl)pyridine (see Compound 31, Step 1) (0.200 g, 0.701 mmol) and methyl 6-bromo-[1,2,4]triazolo[1,5-a]pyridine-7-carboxylate (0.251 g, 0.980 mmol) was dissolved in 1,4-Dioxane (4.0 mL) at rt followed by the addition of CsCO₃ (0.629 g, 1.98 mmol) and water (0.5 mL). The reaction mixture was purged with N₂ gas for 15 min followed by addition of PdCl₂(dppf) (57.0 mg, 0.0701 mmol) followed by a second N₂ purge for 5 min. The reaction mixture was stirred at 90 °C for 1 h. The resulting mixture was poured in water and extracted with EtOAc (3×10 mL). The combined organic layers were dried over Na₂SO₄, filtered, and concentrated under vacuum. The residue was purified by silica gel chromatography eluting with EtOAc (0 to 60%) in hexanes. The pure fractions were collected and concentrated to afford methyl 6-(2-(difluoromethyl)-5-methoxypyridin-4-yl)-[1,2,4]triazolo[1,5-a]pyridine-7-carboxylate (80.0 mg, 34% yield). LCMS m/z 335.1 [M+H]⁺.

### Step 2/Compound 36

To methyl 6-(2-(difluoromethyl)-5-methoxypyridin-4-yl)-[1,2,4]triazolo[1,5-a]pyridine-7-carboxylate (80.0 mg, 0.238 mmol) and Intermediate 9 (39.0 mg, 0.238 mmol) in THF (1 mL) was added 1,5,7-triazabicyclo[4.4.0]dec-5-ene (165 mg, 1.19 mmol). The reaction mixture was stirred at 70 °C for 1 h. The reaction mixture was quenched in water (10 mL) and extracted with EtOAc (3×10 mL). The combined organic layers were dried over Na₂SO₄, filtered, and concentrated under vacuum. The residue was purified by reverse phase HPLC eluting with a gradient of CH₃CN (10 to 100%) in water both containing 0.1% formic acid. Pure fractions were combined and lyophilized to afford Compound 36 (12.0 mg, 11% yield)¹H NMR (400 MHz, DMSO-d₆) δ 13.66 (s, 1H), 9.25 (s, 1H), 8.74 (s, 1H), 8.43 (s, 1H), 8.38 (s, 1H), 7.86 (s, 1H), 6.99 (t, J=55.2 Hz, 1H), 1.70 (bs, 1H), 1.01-1.00 (m, 2H), 0.88 (s, 2H). LCMS m/z 468.2 [M+H]⁺.

### Compound 37/Method D/N-(5-(cyclopropylethynyl)-1,3,4-thiadiazol-2-yl)-2′-(difluoromethyl)-5′-methoxy-1-methyl-6-oxo-1,6-dihydro-[3,4′-bipyridine]-4-carboxamide

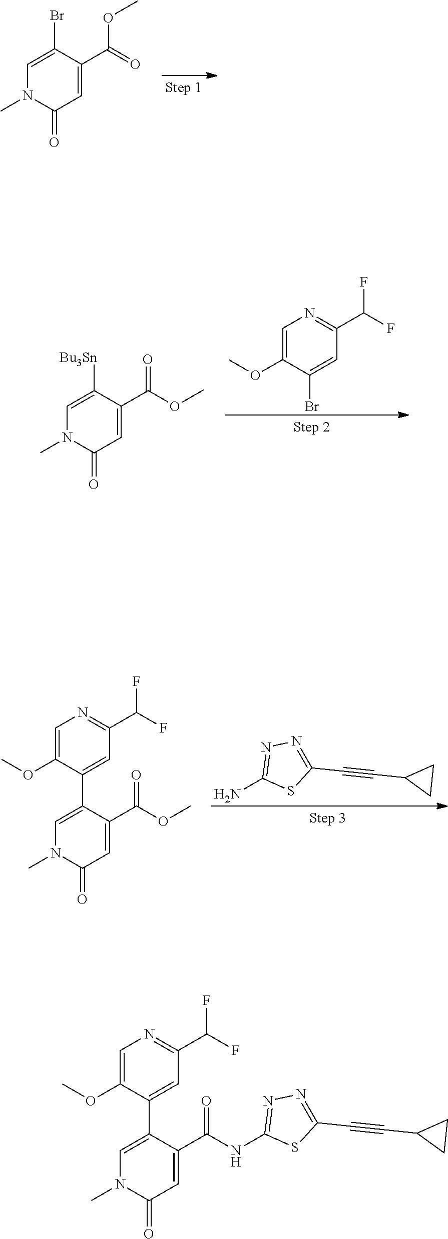

### Step 1/methyl 1-methyl-2-oxo-5-(tributylstannyl)-1,2-dihydropyridine-4-carboxylate

To a solution of methyl 5-bromo-1-methyl-2-oxo-1,2-dihydropyridine-4-carboxylate (2.60 g, 10.6 mmol) in dioxane (7 mL) was added Bu₃SnSnBu₃ (9.10 g, 15.9 mmol). The resulting mixture was purged with N₂ gas for 15 min followed by addition of PdCl₂(dppf) (0.862 g, 1.06 mmol). The reaction mixture was stirred at 100 °C for 8. The resulting mixture was quench in ice cold water and extracted with EtOAc (3×30 mL). The combined organic layers were dry over Na₂SO₄, filtered and concentrated. The residue was purified by silica gel chromatography eluting with EtOAc (40%) in hexanes. Appropriate fractions were combined and concentrated in vacuo to afford methyl 1-methyl-2-oxo-5-(tributylstannyl)-1,2-dihydropyridine-4-carboxylate (2.50 g, 52% yield). ¹H NMR (400 MHz, DMSO-d₆) δ 7.47 (s, 1H), 6.97 (s, 1H), 3.84 (s, 3H), 3.48 (s, 3H), 1.48-1.40 (m, 6H), 1.31-1.24 (m, 6H), 0.98 (t, J=8.0 Hz, 9H), 0.84 (t, J=7.6 Hz, 9H).

### Step 2/methyl 2′-(difluoromethyl)-5′-methoxy-1-methyl-6-oxo-1,6-dihydro-[3,4′-bipyridine]-4-carboxylate

To the solution of 1-methyl-2-oxo-5-(tributylstannyl)-1,2-dihydropyridine-4-carboxylate (0.750 g, 1.64 mmol) in DMF (7.5 mL) was added 4-bromo-2-(difluoromethyl)-5-methoxypyridine (0.313 g, 1.31 mmol). The mixture was purged with N₂ gas for 15 min followed by addition of LiCl (70.0 mg, 1.64 mmol), CuI (31.0 mg, 0.164 mmol) and Pd(PPh₃)₄ (189 mg, 0.164 mmol). The reaction mixture was stirred at 100 °C for 1 h. The resulting mixture was quenched in water (25 mL) and extracted with EtOAc (3×25 mL). The combined organic layers were dried over Na₂SO₄, filtered, and concentrated under vacuum. The residue was purified by silica gel chromatography eluting with MeOH (5%) in DCM. Appropriate fractions were combined and concentrated to afford methyl 2′-(difluoromethyl)-5′-methoxy-1-methyl-6-oxo-1,6-dihydro-[3,4′-bipyridine]-4-carboxylate (256 mg, 48% yield). LCMS m/z 324.8 [M+H]⁺.

### Step 3/Compound 37

To methyl 2′-(difluoromethyl)-5′-methoxy-1-methyl-6-oxo-1,6-dihydro-[3,4′-bipyridine]-4-carboxylate (0.100 g, 0.370 mmol) and Intermediate 9 (48.0 mg, 0.296 mmol) in THF (3 mL) was added 1,5,7-triazabicyclo[4.4.0]dec-5-ene (257 mg, 1.85 mmol). The reaction mixture was stirred at 50 °C for 2 h. The reaction mixture was quenched in water (10 mL) and extracted with EtOAc (3×10 mL). The combined organic layers were dried over Na₂SO₄, filtered, and concentrated under vacuum. The residue was purified by silica gel chromatography eluting with MeOH (4%) in DCM. Appropriate fractions were combined and concentrated to afford Compound 37 (40.0 mg, 24% yield). ¹H NMR (400 MHz, DMSO-d₆) δ 13.50 (s, 1H), 8.30 (s, 1H), 8.033 (s, 1H), 7.68 (s, 1H), 6.95 (t, J=55.2 Hz, 1H), 6.81 (s, 1H), 1.71-1.68 (m, 1H), 1.00-0.99 (m, 2H), 0.87 (s, 2H). LCMS m/z 458.0 [M+H]⁺.

### Compound 38/Method E/2′-chloro-N-(5-(cyclopropylethynyl)thiazol-2-yl)-5′-methoxy-[3,4′-bipyridine]-4-carboxamide

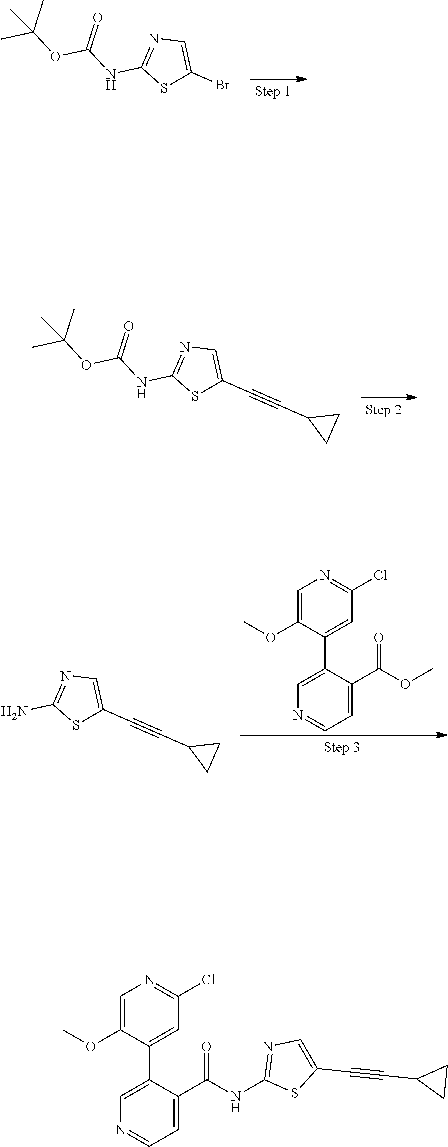

### Step 1/tert-butyl (5-(cyclopropylethynyl)thiazol-2-yl)carbamate

CuI (27.4 mg, 143 μmol), NEt₃ (605 uL, 4.30 mmol) and N-Boc-2-Amino-5-bromothiazole (509 uL, 1.43 mmol) were combined in DMF (8.5 mL). N₂ was bubbled in the mixture for 10 min then Pd(PPh₃)₄ (167 mg, 143 μmol) and Cyclopropylacetylene (728 uL, 8.60 mmol) were added. The reaction mixture was stirred at 50 °C for 12 hrs. Silica gel was added to the reaction mixture and the volatiles were concentrated under vacuum. The dry pack was purified by silica gel chromatography eluting with EtOAc (5 to 80%) in Hexanes. Appropriate fractions were combined and concentrated to afford tert-butyl (5-(cyclopropylethynyl)thiazol-2-yl)carbamate (226 mg, 60% yield) as an orange solid. LCMS m/z 266.0 [M+H]⁺.

### Strep 2/5-(cyclopropylethynyl)thiazol-2-amine

Tert-butyl (5-(cyclopropylethynyl)thiazol-2-yl)carbamate (200 mg, 757 μmol) was dissolved in DCM (1 mL) and trifluoroacetic acid (585 uL, 7.57 mmol) was added. The mixture was stirred for 2 hrs at rt. The mixture was concentrated, dissolved with DCM, and washed with aqueous saturated NaHCO₃. The organic phase was separated, and the aqueous phase was back extracted DCM (3×). The organic phases were combined, dried over Na₂SO₄, filtered and concentrated to afford 5-(cyclopropylethynyl)thiazol-2-amine (100 mg, 80%) as an orange solid. The product was used in the next step without further purification. LCMS m/z 166.0[M+H]⁺.

### Step 3/Compound 38

2′-chloro-5′-methoxy-[3,4′-bipyridine]-4-carboxylate (see Intermediate 15, Step 1) (100 mg, 360 μmol) and 5-(cyclopropylethynyl)thiazol-2-amine (70.7 mg, 431 μmol) were dissolved in THF (1.8 mL). 1,5,7-triazabicyclo[4.4.0]dec-5-ene (255 mg, 1.80 mmol) was added and the mixture was stirred at 90 °C for 4 hrs. EtOAc and an aqueous saturated solution of NH₄Cl were added. The aqueous phase was back extracted twice with EtOAc. The combined organic layers were concentrated, dissolved in DMSO (1 mL), and filtered. The solution was purified by preparative HPLC eluting with CH₃CN (30-50%) in 10 mM aqueous NH₄HCOOH. (Column: Waters CSH C18 OBD Prep Column, 5 μm, 30 mm×75 mm). The appropriate fractions were combined and lyophilized to afford Compound 38 (6.6 mg, 5%) as a yellow solid. ¹H NMR (400 MHz, DMSO-d₆) δ 13.02 (s, 1H), 8.81 (d, J=5.0 Hz, 1H), 8.69 (s, 1H), 8.12 s, 1H), 7.72 (d, J=5.0 Hz, 1H), 7.62 (s, 1H), 7.59 (s, 1H), 3.54 (s, 3H), 1.60-1.54 (m, 1H), 0.93-0.84 (m, 2H), 0.77-0.67 (m, 2H). LCMS m/z 411.8 [M+H]⁺.

### Compound 108/Method D N-(5-(cyclopropylethynyl)-1,3,4-thiadiazol-2-yl)-2-(2-(difluoromethyl)-5-methoxypyridin-4-yl)-4-(3-oxomorpholino)benzamide

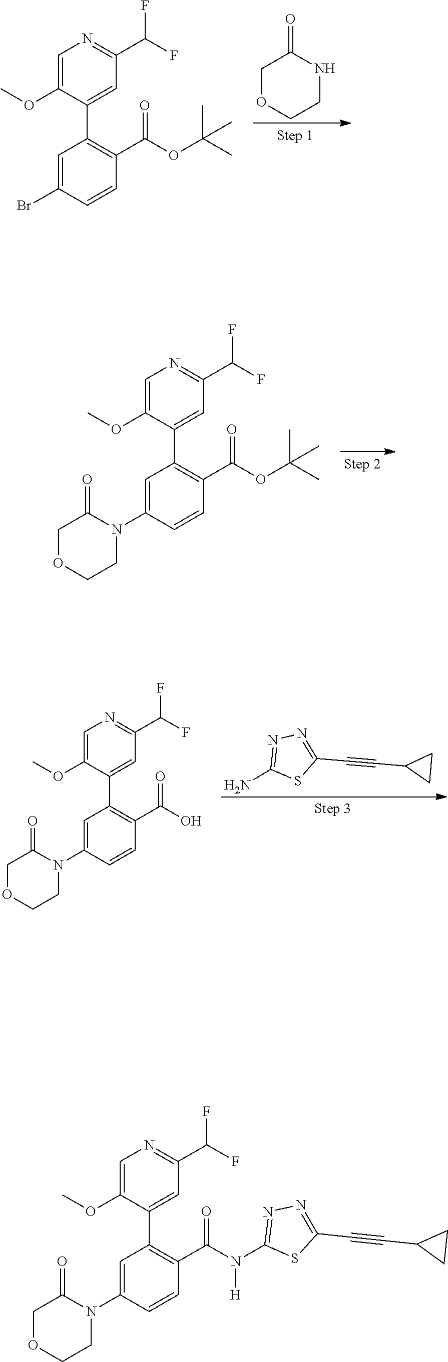

### Step 1/tert-butyl 2-(2-(difluoromethyl)-5-methoxypyridin-4-yl)-4-(3-oxomorpholino)benzoate

A microwave vial was charged with Intermediate 27 (600 mg, 1.45 mmol), morpholin-3-one (366 mg, 3.62 mmol), dioxane (6 mL) and Xantphos Pd G3 (135 mg, 142.2 μmol). Cesium carbonate (945 mg, 2.90 mmol) was added and the vessel was flushed with N₂, sealed and stirred at 90 °C overnight. The reaction mixture was diluted with DCM and adsorbed on silica. The residue was purified by silica gel chromatography eluting with a gradient of EtOAc (30 to 100%) in heptane to provide tert-butyl 2-(2-(difluoromethyl)-5-methoxypyridin-4-yl)-4-(3-oxomorpholino)benzoate (480 mg, 1.10 mmol, 76% yield). LCMS m/z 435.2 [M+H]⁺.

### Step 2/2-(2-(difluoromethyl)-5-methoxypyridin-4-yl)-4-(3-oxomorpholino)benzoic acid

A solution of tert-butyl 2-(2-(difluoromethyl)-5-methoxypyridin-4-yl)-4-(3-oxomorpholino)benzoate (480 mg, 1.10 mmol) in DCM (2.5 mL) was treated with HCl 4M in Dioxane (5 mL). The solution was stirred at rt 2 days. The reaction mixture was concentrated in vacuo to afford crude 2-(2-(difluoromethyl)-5-methoxypyridin-4-yl)-4-(3-oxomorpholino)benzoic acid (418 mg) which was used in the next step without further purification. LCMS m/z 379.3 [M+H]⁺.

### Step 3/Compound 108

A solution of 2-(2-(difluoromethyl)-5-methoxypyridin-4-yl)-4-(3-oxomorpholino)benzoic acid (418 mg, 1.10 mmol) and Intermediate 9 (192 mg, 1.16 mmol) in pyridine (6 mL) was treated with EDC (320 mg, 1.67 mmol). The mixture was stirred at rt overnight. The mixture was diluted with EtOAc and water. The layers were separated, and the aqueous layer was extracted twice with EtOAc. The combined organic layers were washed with brine, dried over MgSO₄, filtered and concentrated. The residue was purified by silica gel chromatography eluting with a gradient of EtOAc (40 to 100%) in heptane to provide Compound 108 (402 mg, 69% yield). ¹H NMR (DMSO-d₆) δ: 13.27 (s, 1H), 8.38 (s, 1H), 7.85 (d, J=8.4 Hz, 1H), 7.68 (dd, J=8.4, 2.1 Hz, 1H), 7.64 (s, 1H), 7.59 (d, J=2.1 Hz, 1H), 6.97 (t, J=55.1 Hz, 1H), 4.25 (s, 2H), 4.06-3.97 (m, 2H), 3.92-3.84 (m, 2H), 3.62 (s, 3H), 1.75-1.63 (m, 1H), 1.06-0.96 (m, 2H), 0.90-0.83 (m, 2H). LCMS m/z 526.2 [M+H]⁺.

### Compound 109/Method D/N-(5-(cyclopropylethynyl)-1,3,4-thiadiazol-2-yl)-2-(2-(difluoromethyl)-5-methoxypyridin-4-yl)-4-(4-methyl-2-oxopiperazin-1-yl)benzamide

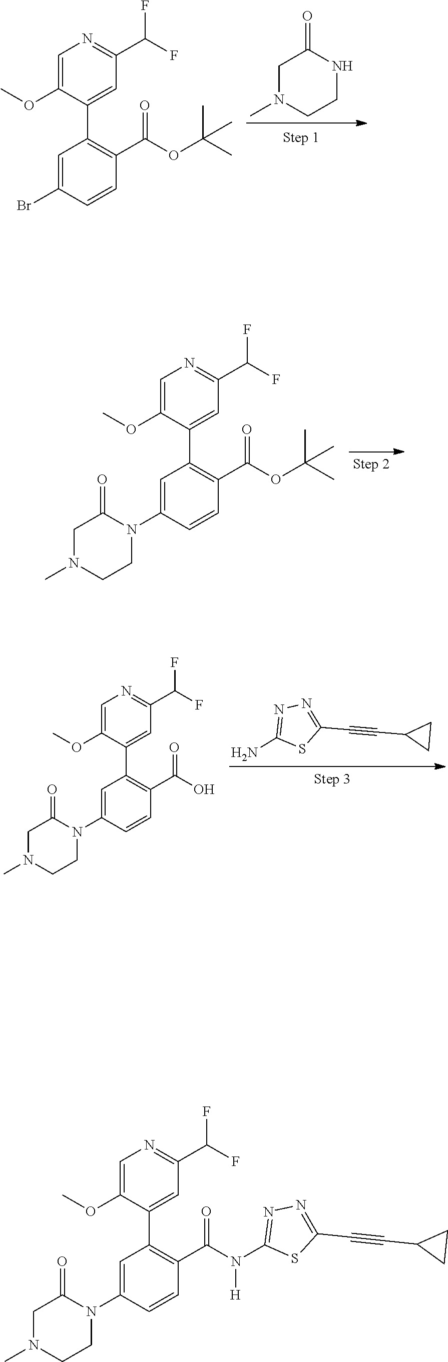

### Step 1/tert-butyl 2-(2-(difluoromethyl)-5-methoxypyridin-4-yl)-4-(4-methyl-2-oxopiperazin-1-yl)benzoate

To a solution of Intermediate 27 (650 mg, 1.57 mmol) in dioxane (6 mL) were added Palladium(II) acetate (36.0 mg, 160 umol), 4-methylpiperazin-2-one (205 mg, 1.80 mmol), Xantphos (138 mg, 239 μmol) and Cs₂CO₃ (1.02 g, 3.14 mmol). The mixture was degassed in vacuo and then backfilled with N₂ in a sealed vial. The resulting mixture was stirred at 90 °C under N₂ for 1 hr. After cooling to rt, the mixture was diluted with water and extracted with EtOAc (3×25 mL). The combined organic extracts were washed with brine, dried over Na₂SO₄, filtered, and concentrated to dryness. The residue was purified on silica gel chromatography eluting with MeOH (0-10%) in DCM (both solvents contain 0.1% TEA) to provide tert-butyl 2-(2-(difluoromethyl)-5-methoxypyridin-4-yl)-4-(4-methyl-2-oxopiperazin-1-yl)benzoate (480 mg, 68% yield) as an off-white solid. LCMS m/z 448.4 [M+H].

### Step 2/2-(2-(difluoromethyl)-5-methoxypyridin-4-yl)-4-(4-methyl-2-oxopiperazin-1-yl)benzoic acid

The solution of tert-butyl 2-(2-(difluoromethyl)-5-methoxypyridin-4-yl)-4-(4-methyl-2-oxopiperazin-1-yl)benzoate (480 mg, 1.07 mmol) in HCl 4M in dioxane (5 mL) was stirred at rt for 2 days. The volatiles were removed in vacuo to provide 2-(2-(difluoromethyl)-5-methoxypyridin-4-yl)-4-(4-methyl-2-oxopiperazin-1-yl)benzoic acid (419 mg, 1.07 mmol, 99.81% yield) as an off-white solid which was used in the next step without further purification.

### Step 3/Compound 109

To a solution of 2-(2-(difluoromethyl)-5-methoxypyridin-4-yl)-4-(4-methyl-2-oxopiperazin-1-yl)benzoic acid (467 mg, 1.19 mmol) and Intermediate 9 (200 mg, 1.21 mmol) in pyridine (1.5 mL) was added EDC (550 mg, 2.87 mmol). The reaction was stirred at rt for 18 hr. The crude reaction mixture was concentrated to dryness in vacuo. The residue was dissolved in DMSO, filtered, and the filtrate was purified by reverse phase flash chromatography eluting with a gradient of CH₃CN (20 to 100%) in water both containing 0.1% formic acid. Appropriate fractions were combined and lyophilized to afford Compound 109 (180 mg, 28% yield) as an off-white solid. ¹H NMR (400 MHz, DMSO-d₆) δ 13.22 (s, 1H), 8.33 (s, 1H), 7.79 (d, J=8.3 Hz, 1H), 7.59 (s, 1H), 7.56 (m, 1H), 7.48 (d, J=2.1 Hz, 1H), 6.92 (t, J=55.1 Hz, 1H), 3.85-3.67 (m, 2H), 3.57 (s, 3H), 3.11 (s, 2H), 2.79-2.68 (m, 2H), 2.25 (s, 3H), 1.65 (m, 1H), 0.99-0.93 (m, 2H), 0.85-0.73 (m, 2H). LCMS m/z 539.4 [M+H]⁺.

### Compound 110/Method D/N-(5-(cyclopropylethynyl)-1,3,4-thiadiazol-2-yl)-2′-(difluoromethyl)-5′-methoxy-6-(3-oxomorpholino)-[4,4′-bipyridine]-3-carboxamide

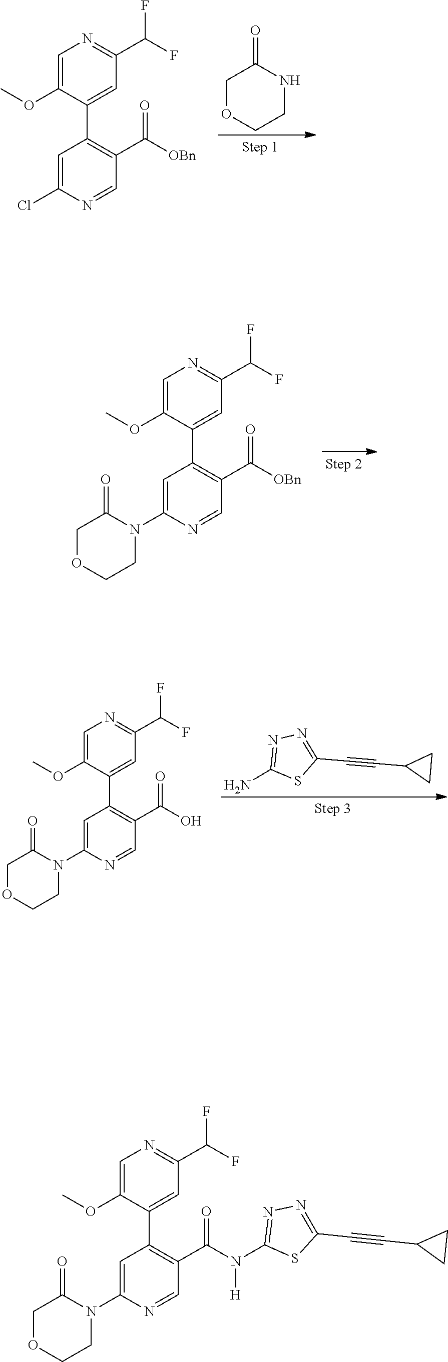 [SMILES: COc1cnc(C(F)F)cc1-c1cc(Cl)ncc1C(=O)OCc1ccccc1 ; O=C1COCCN1 ; COc1cnc(C(F)F)cc1-c1cc(N2CCOCC2=O)ncc1C(=O)OCc1ccccc1 ; COc1cnc(C(F)F)cc1-c1cc(N2CCOCC2=O)ncc1C(=O)O ; Nc1nnc(C#CC2CC2)s1 ; COc1cnc(C(F)F)cc1-c1cc(N2CCOCC2=O)ncc1C(=O)Nc1nnc(C#CC2CC2)s1]

### Step 1/benzyl 2′-(difluoromethyl)-5′-methoxy-6-(3-oxomorpholino)-[4,4′-bipyridine]-3-carboxylate

Intermediate 25 (10.84 g, 26.78 mmol), morpholin-3-one (3.25 g, 32.17 mmol), Pd(OAc)₂ (600 mg, 2.67 mmol), XantPhos (2.33 g, 4.02 mmol) and Cs₂CO₃ (17.4 g, 53.5 mmol) were combined in Dioxane (150 mL) flushed with N₂, sealed and stirred at 80 °C for 1 h. The cooled reaction mixture was filtered rinsed with EtOAc and concentrated in vacuo. The residue was purified by silica gel chromatography eluting with a gradient of EtOAc (20 to 100%) in hexanes to provide benzyl 2′-(difluoromethyl)-5′-methoxy-6-(3-oxomorpholino)-[4,4′-bipyridine]-3-carboxylate (10.84 g, 86% yield). ¹H NMR (DMSO-d₆) δ: 8.95 (d, J=0.6 Hz, 1H), 8.40 (s, 1H), 8.14 (s, 1H), 7.58 (s, 1H), 7.35-7.27 (m, 3H), 7.15-7.11 (m, 2H), 7.00 (d, J=54.5 Hz, 1H), 5.15 (s, 2H), 4.29 (s, 2H), 4.03 (dtt, J=6.0, 4.2, 2.2 Hz, 4H), 3.73 (s, 3H). LCMS m/z 470.2 [M+H]⁺.

### Step 2/2′-(difluoromethyl)-5′-methoxy-6-(3-oxomorpholino)-[4,4′-bipyridine]-3-carboxylic acid

A solution of benzyl 2′-(difluoromethyl)-5′-methoxy-6-(3-oxomorpholino)-[4,4′-bipyridine]-3-carboxylate (8.70 g, 22.9 mmol) In EtOH (225 mL) and DCM (125 mL) was treated with Pd on Carbon 10% wt (1.08 g) and stirred under an atmosphere of hydrogen for 24 h. Additional Pd on Carbon 10% wt (1.08 g) was added to complete the reaction under an atmosphere of hydrogen for 24 hrs. The reaction mixture was flushed with N₂ then filtered over Celite (filter cake was rinsed with 20% MeOH/DCM) and the filtrated was concentrated to give 4-[2-(difluoromethyl)-5-methoxy-4-pyridyl]-6-(3-oxomorpholin-4-yl)pyridine-3-carboxylic acid (8.70 g, 99% yield) which was used in the next step without further purification. ¹H NMR (DMSO-d₆) δ: 13.11 (s, 1H), 8.91 (d, J=0.7 Hz, 1H), 8.53 (s, 1H), 8.11 (d, J=0.7 Hz, 1H), 7.55 (s, 1H), 6.97 (t, J=55.1 Hz, 1H), 4.29 (s, 2H), 4.09-3.98 (m, 4H), 3.87 (s, 3H). LCMS m/z 380.3 [M+H]⁺.

### Step 3/Compound 110

A solution of 2′-(difluoromethyl)-5′-methoxy-6-(3-oxomorpholino)-[4,4′-bipyridine]-3-carboxylic acid (175 mg, 461 μmol) and Intermediate 9 (115 mg, 696.06 μmol) in pyridine (2.5 mL) was treated with EDC (220 mg, 1.15 mmol). The reaction mixture was stirred at rt overnight, then diluted with EtOAc and water. The organic layer was washed with brine, dried over MgSO₄, filtered and concentrated. The residue was purified by silica gel chromatography eluting with a gradient of EtOAc (40 to 100%) in heptane to provide a solid which was lyophilized in ACN/water to provide Compound 110 (130 mg, 54% yield). ¹H NMR (DMSO-d₆) δ: 13.48 (s, 1H), 8.87 (s, 1H), 8.47 (s, 1H), 8.19 (s, 1H), 7.65 (s, 1H), 7.01 (t, J=55.0 Hz, 1H), 4.32 (s, 2H), 4.11-3.99 (m, 4H), 3.66 (s, 3H), 1.70 (tt, J=8.2, 5.0 Hz, 1H), 0.99 (dt, J=8.3, 3.2 Hz, 2H), 0.91-0.83 (m, 2H). LCMS m/z 527.3 [M+H]⁺.

### Compound 111/Method D/N-(5-(cyclopropylethynyl)-1,3,4-thiadiazol-2-yl)-2-(2-(difluoromethyl)-5-methoxypyridin-4-yl)-4-(5-methyl-1,3,4-oxadiazol-2-yl)benzamide

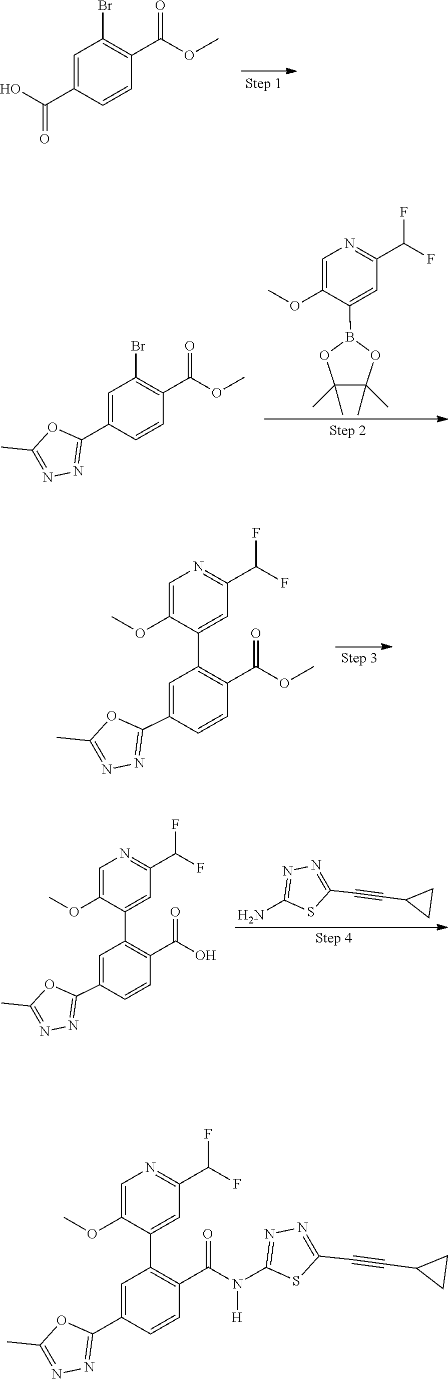

### Step 1/methyl 2-bromo-4-(5-methyl-1,3,4-oxadiazol-2-yl)benzoate

In a 1 L RBF loaded with 3-bromo-4-methoxycarbonyl-benzoic acid (25.0 g, 96.5 mmol), DMF (0.35 mL, 4.52 mmol,) was added to thionyl chloride (175 mL, 2.41 mol) and the brown suspension was refluxed for 3 hrs. Reaction progress was monitored by adding one drop of the reaction mixture to a LCMS vial containing a few drops of propylamine in acetonitrile. The volatiles were removed under reduced pressure and remaining thionyl chloride in the residue was remove by one co-evaporation with acetonitrile to yield methyl 2-bromo-4-chlorocarbonyl-benzoate as an orange residue which was used without any further purification. The latter was dissolved in DCM (250 mL) to which pyridine (23 mL, 284 mmol) was added followed by the portion wise addition of tert-butyl N-aminocarbamate (13.2 g, 99.9 mmol) in four equal portions (exotherm observed leading to DCM boiling). The orange solution was stirred for 10 min after which LCMS analysis suggested an almost complete conversion to the desired product tert-butyl 2-(3-bromo-4-(methoxycarbonyl)benzoyl)hydrazine-1-carboxylate. Trifluoroacetic acid (200 mL, 2.60 mol) was then added dropwise, and the resulting solution was stirred at rt for 60 min. LCMS indicate complete conversion to methyl 2-bromo-4-(hydrazinecarbonyl)benzoate. Triethyl ortho acetate (41.0 mL, 223 mmol) was added and the orange suspension was heated to 50 °C for 45 min after which more triethylortho acetate (8 mL, 44.47 mmol,) was added to push reaction to completion and stirred for 10 min. The volatiles were removed under reduced pressure to yield a thick orange suspension. 400 mL of water was added under vigorous stirring and the orange solution was extracted with EtOAc (2×). The combined organic layers were washed with brine, dried over Na₂SO₄ and concentrated under vacuum. The orange oil was diluted with a minimum of dichloromethane and purified by silica gel chromatography eluting with a gradient of EtOAc (40 to 65%) in hexanes to afford the desired product as a white solid. Mixed fractions were repurified on a second silica gel chromatography eluting with a gradient of EtOAc (0 to 30%) in DCM. The appropriate fractions from both purifications were combined to afford methyl 2-bromo-4-(5-methyl-1,3,4-oxadiazol-2-yl)benzoate (24.5 g, 76.9% yield, 90% purity) as a white solid. ¹H NMR (400 MHz, CD₃Cl) δ 8.23 (m, 1H), 7.95 (m, 1H), 7.85 (m, 1H), 3.91 (t, J=2.6 Hz, 3H), 2.66 (t, J=1.9 Hz, 3H). LCMS m/z 297.0 [M+H]⁺.

### Step 2/methyl 2-(2-(difluoromethyl)-5-methoxypyridin-4-yl)-4-(5-methyl-1,3,4-oxadiazol-2-yl)benzoate

N₂ was bubbled 5 min through for in a suspension of Intermediate 19 (39.0 g, 137 mmol), methyl 2-bromo-4-(5-methyl-1,3,4-oxadiazol-2-yl)benzoate (36.0 g, 109 mmol) from, K₂CO₃ (2 M, 164 mL, 328 mmol), and Pd(dppf)Cl₂·DCM (8.91 g, 10.9 mmol) in dioxane (450 mL) and water (157 mL). The reaction mixture was stirred at 75 °C for 15 min under a N₂ atmosphere. The mixture was extracted with EtOAc (2×). The combined organic layers were washed with brine, dried over Na₂SO₄ and the volatiles were evaporated under reduced pressure. The brown residue was taken in EtOAc and filtered through a Celite pad. The filtrate was evaporated under reduced pressure and the residue was taken in a minimum of dichloromethane and purified by silica gel chromatography eluting with a gradient of EtOAc (30 to 100%) in heptane to afford methyl 2-(2-(difluoromethyl)-5-methoxypyridin-4-yl)-4-(5-methyl-1,3,4-oxadiazol-2-yl)benzoate (39.5 g, 97% yield) as a pale orange solid. ¹H NMR (400 MHz, CDCl3) δ 8.26 (s, 1H), 8.12-8.01 (m, 2H), 7.92 (d, J=1.8 Hz, 1H), 7.51 (s, 1H), 6.62 (t. J=55.7 Hz, 1H), 3.82 (s, 3H), 3.66 (s, 3H), 2.58 (s, 3H). LCMS m/z 376.1 [M+H]⁺.

### Step 3/2-(2-(difluoromethyl)-5-methoxypyridin-4-yl)-4-(5-methyl-1,3,4-oxadiazol-2-yl)benzoic acid

Methyl 2-(2-(difluoromethyl)-5-methoxypyridin-4-yl)-4-(5-methyl-1,3,4-oxadiazol-2-yl)benzoate (39.5 g, 101 mmol) was dissolved in MeOH (110 mL) and dioxane (265 mL) to yield an orange solution before the addition of LiOH·H₂O (8.50 g, 203 mmol) In water (90 mL) resulting in a brown suspension. The suspension was heated to 60 °C for 1 h. MeOH and dioxane were removed under reduced pressure, and hydrochloride (1 M, 225 mL) was added slowly until pH-3 to yield a milky suspension. The solid was recovered by filtration and dried 48 hrs on the high vacuum pump to afford 2-(2-(difluoromethyl)-5-methoxypyridin-4-yl)-4-(5-methyl-1,3,4-oxadiazol-2-yl)benzoic acid (26.5 g, 73% yield) as a beige solid. ¹H NMR (400 MHz, CDCl₃) δ 8.26 (s, 1H), 8.21-8.10 (m, 2H), 7.95 (s, 1H), 7.58 (s, 1H), 6.66 (t, J=55.5 Hz, 1H), 3.80 (s, 3H), 2.62 (s, 3H). LCMS m/z 362.1 [M+H]⁺.

### Step 4/Compound 111

2-(2-(difluoromethyl)-5-methoxypyridin-4-yl)-4-(5-methyl-1,3,4-oxadiazol-2-yl)benzoic acid (25.0 g, 69.2 mmol) and Intermediate 9 (13.5 g, 81.71 mmol) were suspended in Pyridine (150 mL) and EDC (28.33 g, 147.8 mmol) was added. The beige suspension was stirred at rt for 2 hrs. LCMS analysis indicated incomplete conversion. The reaction was stirred for an additional 16 hrs. Most of the pyridine was removed in vacuo to yield a thick brown oil to which water was added slowly under vigorous stirring and a beige precipitate was observed. The solid was collected by filtration, washed with H₂O (3×), washed with EtOH (3×) and air-dried to afford a beige solid (36.0 g) that was kept aside. The ethanol washes were combined, evaporated, taken in a minimum of 10% MeOH in DCM, silica gel was added, the volatiles were evaporated under reduced vacuum and the residue was purified by silica gel chromatography (dry load) eluting with a gradient of EtOAc (0 to 100%) in DCM. Appropriate fractions were combined and concentrated in vacuo to afford a pale-yellow solid (2.10 g). Solids were combined dried in vacuum oven at 45 °C for 20 hrs to yield Compound 111 (30.3 g, 86% yield, 100% purity) as a beige solid. ¹H NMR (400 MHz, DMSO-d₆) δ 13.44 (s, 1H), 8.38 (s, 1H), 8.14 (dd, J=8.1, 1.8 Hz, 1H), 8.04-7.91 (m, 2H), 7.71 (s, 1H), 6.95 (t, J=55.1 Hz, 1H), 3.60 (s, 3H), 2.57 (s, 3H), 1.65 (tt, J=8.3, 5.1 Hz, 1H), 0.94 (m, 2H), 0.88-0.75 (m, 2H). LCMS m/z 509.2 [M+H]⁺.

### Compound 112/Method D/4-(cyanomethyl)-N-(5-(cyclopropylethynyl)-1,3,4-thiadiazol-2-yl)-2-(2-(difluoromethyl)-5-methoxypyridin-4-yl)benzamide

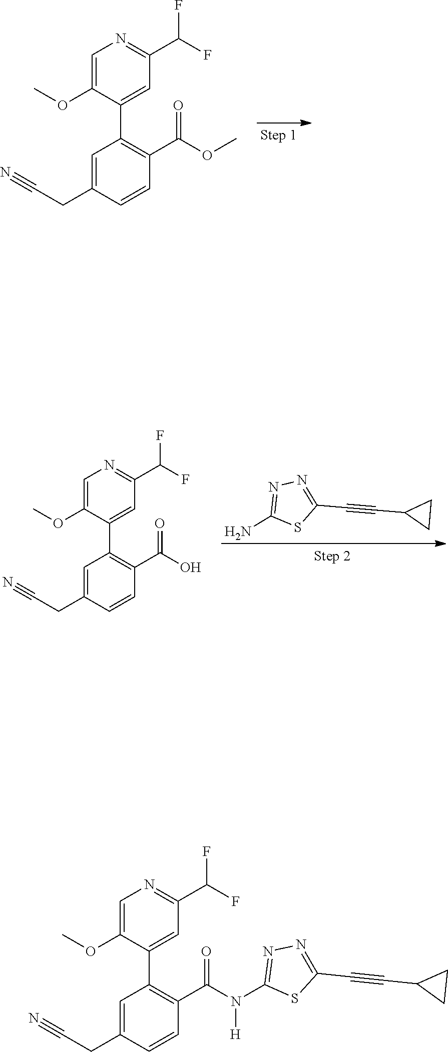 [SMILES: COC(=O)c1ccc(CC#N)cc1-c1cc(C(F)F)ncc1OC ; COc1cnc(C(F)F)cc1-c1cc(CC#N)ccc1C(=O)O ; Nc1nnc(C#CC2CC2)s1 ; COc1cnc(C(F)F)cc1-c1cc(CC#N)ccc1C(=O)Nc1nnc(C#CC2CC2)s1]

### Step 1/4-(cyanomethyl)-2-(2-(difluoromethyl)-5-methoxypyridin-4-yl)benzoic acid

A solution of Intermediate 33 (877 mg, 2.34 mmol) in MeOH (7 mL) and Dioxane (28 mL) was treated with LiOH (1 M, 7.0 mL, 7 mmol). The mixture was stirred at 50 °C for 3 hrs. The reaction mixture was neutralized with 10% HCl aq solution to pH 5. The volatiles were evaporated, and the remaining residue was dissolved in EtOAc. The organic layer was washed with brine, dried over MgSO₄, filtered and concentrated to give 4-(cyanomethyl)-2-(2-(difluoromethyl)-5-methoxypyridin-4-yl)benzoic acid (890 mg) which was used as such in the next step without further purification. LCMS m/z 319.2 [M+H]⁺.

### Step 2/Compound 112

A solution of 4-(cyanomethyl)-2-(2-(difluoromethyl)-5-methoxypyridin-4-yl)benzoic acid (481 mg, 1.51 mmol) and Intermediate 9 (275 mg, 1.66 mmol) In pyridine (10 mL) was treated with EDC (435 mg, 2.27 mmol). The mixture was stirred at rt for 2 h. The reaction mixture was diluted with EtOAc and washed with 10% aqueous HCl. The organic layer was washed with brine, dried over MgSO₄, filtered and concentrated. The residue was purified by silica gel chromatography eluting with a gradient of EtOAc (20 to 100%) in heptane to provide the desired product. The material was repurified by preparative HPLC eluting with a gradient of CH₃CN (40 to 70%) in water both containing 0.1% formic acid. Appropriate fractions were combined and lyophilized to afford Compound 112 (131 mg, 19% yield)¹H NMR (DMSO-d₆) δ:13.28 (s, 1H), 8.39 (s, 1H), 7.85 (d, J=7.9 Hz, 1H), 7.63 (s, 1H), 7.60 (dd, J=8.0, 1.8 Hz, 1H), 7.48 (d, J=1.8 Hz, 1H), 6.98 (t, J=55.1 Hz, 1H), 4.20 (s, 2H), 3.62 (s, 3H), 1.69 (tt, J=8.2, 5.1 Hz, 1H), 1.05-0.96 (m, 2H), 0.90-0.83 (m, 2H). LCMS m/z 466.2 [M+H]⁺.

### Compound 113/Method D/N-(5-(cyclopropylethynyl)-1,3,4-thiadiazol-2-yl)-2′-(difluoromethyl)-5′-methoxy-6-(1-methyl-1H-pyrazol-3-yl)-[4,4′-bipyridine]-3-carboxamide

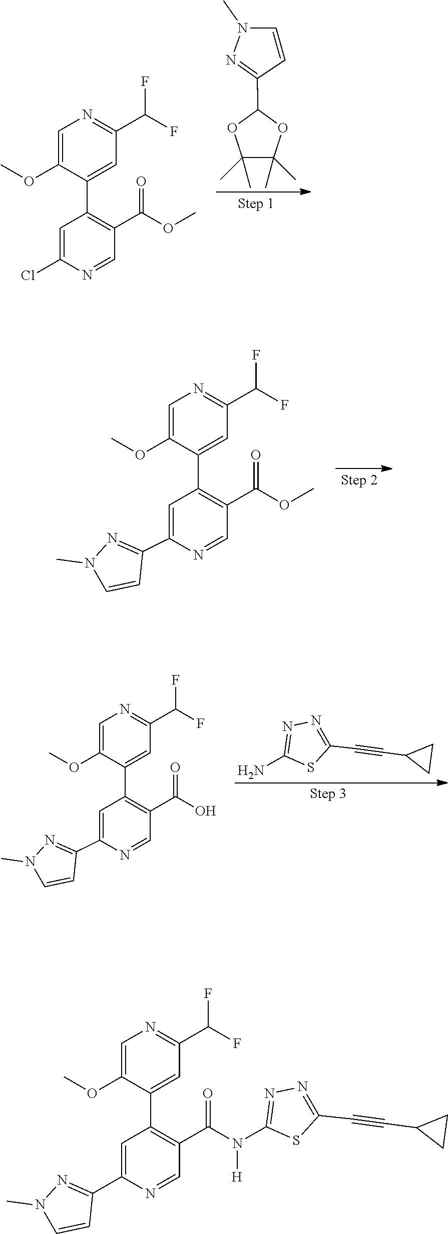

### Step 1/methyl 2′-(difluoromethyl)-5′-methoxy-6-(1-methyl-1H-pyrazol-3-yl)-[4,4′-bipyridine]-3-carboxylate

A RBF was charged with Intermediate 21 (15.5 g, 47.2 mmol) and 1-methyl-3-(4,4,5,5-tetramethyl-1,3,2-dioxaborolan-2-yl)-1H-pyrazole (14.24 g, 68.4 mmol), dioxane (160 mL) and K₂CO₃ (2 M, 47.2 mL, 94.4 mmol). N₂ was bubbled through the mixture under sonication for 10 min, Pd(OAc)₂ (1.59 g, 7.07 mmol) and SPhos (5.81 g, 14.2 mmol) were added, and N₂ was bubbled in the resulting mixture under sonication for another 10 min and then heated to 80 °C for 1 h. The resulting reaction mixture was cooled to rt, filtered on a celite plug, rinsed with EtOAc (300 mL). The resulting solution was diluted with H₂O, layers separated, aqueous layer extracted with EtOAc. The combined organic extracts were washed with brine, dried over Na₂SO₄, filtered on a silica gel plug using EtOAc and adsorbed on silica. The residue was purified by silica gel chromatography (220 g, dry-load) eluting with a gradient of EtOAc (50 to 100%) in Heptane. Appropriate fractions were combined and concentrated in vacuo to afford methyl 2′-(difluoromethyl)-5′-methoxy-6-(1-methyl-1H-pyrazol-3-yl)-[4,4′-bipyridine]-3-carboxylate (12.97 g, 74% yield) as a yellowish white solid. ¹H NMR (400 MHz, CDCl₃) δ 9.16 (d, J=0.7 Hz, 1H), 8.32 (s, 1H), 7.85 (d, J=0.7 Hz, 1H), 7.57 (s, 1H), 7.45 (d, J=2.3 Hz, 1H), 6.97 (d, J=2.3 Hz, 1H), 6.67 (t, J=55.7 Hz, 1H), 3.99 (s, 3H), 3.87 (s, 3H), 3.75 (s, 3H). LCMS m/z 375.1 [M+H]⁺.

### Step 2/2′-(difluoromethyl)-5′-methoxy-6-(1-methyl-1H-pyrazol-3-yl)-[4,4′-bipyridine]-3-carboxylic acid

To a 1 L RBF containing methyl 2-(difluoromethyl)-5′-methoxy-6-(1-methyl-1H-pyrazol-3-yl)-[4,4′-bipyridine]-3-carboxylate (24.22 g, 64.70 mmol) was added 525 mL dioxane, 130 mL MeOH and aqueous LiOH (1M, 130 mL, 130 mmol). The reaction mixture was heated to 60 °C for 1.25 h. The resulting mixture was cooled to rt, 4M aqueous HCl was added dropwise to reach a pH of 4-5. The reaction mixture was concentrated to remove volatiles. The resulting aqueous suspension was stirred for 30-40 min. The solid was collected by filtration, washed with H₂O, air-dried then dried in vacuo to afford 2′-(difluoromethyl)-5′-methoxy-6-(1-methyl-1H-pyrazol-3-yl)-[4,4′-bipyridine]-3-carboxylic acid (23.25 g, 99% yield) as a light beige solid. ¹H NMR (400 MHz, CDCl₃) δ 9.28 (d, J=0.7 Hz, 1H), 8.30 (s, 1H), 7.88 (d, J=0.7 Hz, 1H), 7.59 (s, 1H), 7.45 (d, J=2.3 Hz, 1H), 6.95 (d, J=2.3 Hz, 1H), 6.69 (t, J=55.6 Hz, 1H), 3.99 (s, 3H), 3.86 (s, 3H). LCMS m/z 361.1 [M+H]⁺.

### Step 3/Compound 113

To a mixture of 2′-(difluoromethyl)-5′-methoxy-6-(1-methyl-1H-pyrazol-3-yl)-[4,4′-bipyridine]-3-carboxylic acid (22.0 g, 61.1 mmol) and Intermediate 9 (13.11 g, 79.38 mmol) in pyridine (220 mL) was added EDC (29.9 g, 156 mmol) and the mixture was stirred at rt for 4 h. LCMS revealed a 73% conversion to the desired compound. Additional Intermediate 9 was added (5.53 g, 33.4 mmol) at 4 hrs (2.50 g) and 6 hrs (3.03 g) followed by EDC (5.85 g, 30.5 mmol) at 8 hrs. The resulting mixture was stirred for an additional 12 hrs at rt to reach a 94% conversion by LCMS. The reaction mixture was concentrated to remove pyridine. To the residue stirred and 250 mL H₂O was added dropwise. The resulting mixture was stirred for 1 h at rt. The solid was collected by filtration washed with several portions of H₂O and air-dried. The resulting solid (46 g) was stirred in EtOH (100 mL) at 40 °C until clumps disappeared, cooled to rt, filtered, washed with ice-cold EtOH and air-dried. The resulting solid was transferred to a crystallizing dish and placed in a vacuum oven at 47 °C overnight. The resulting solid (25.84 g) was stirred in acetone (775 mL) at reflux for 1 h, cooled to rt, filtered, air-dried then dried overnight at 48 °C in a vacuum oven to finally afford Compound 113 (25.57 g, 83% yield). ¹H NMR (400 MHz, DMSO-d₆) δ 13.47 (s, 1H), 8.95 (s, 1H), 8.47 (s, 1H), 7.94 (s, 1H), 7.86 (d, J=2.2 Hz, 1H), 7.78 (s, 1H), 7.01 (t, J=55.0 Hz, 1H), 6.96 (d, J=2.3 Hz, 1H), 3.95 (s, 3H), 3.67 (s, 3H), 1.70 (tt, J=8.3, 5.0 Hz, 1H), 0.99 (dt, J=8.2, 3.3 Hz, 2H), 0.88 (dt, J=4.9, 3.1 Hz, 2H). ¹⁹F NMR (376 MHz, DMSO-d₆) δ −113.36 (d, J=55.0 Hz). LCMS m/z 508.1 [M+H]⁺.

### Compound 114/Method E/5-(2-chloro-5-(difluoromethyl)phenyl)-N-(5-(cyclopropylethynyl)thiazol-2-yl)-1-((5-methyl-1,3,4-oxadiazol-2-yl)methyl)-2-oxo-1,2-dihydropyridine-4-carboxamide

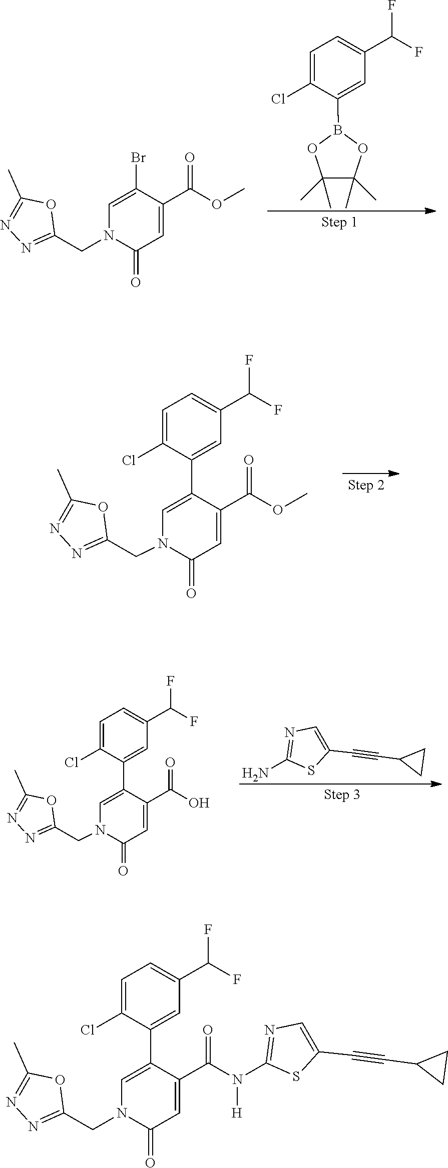 [SMILES: COC(=O)c1cc(=O)n(Cc2nnc(C)o2)cc1Br ; CC1(C)OB(c2cc(C(F)F)ccc2Cl)OC1(C)C ; COC(=O)c1cc(=O)n(Cc2nnc(C)o2)cc1-c1cc(C(F)F)ccc1Cl ; Cc1nnc(Cn2cc(-c3cc(C(F)F)ccc3Cl)c(C(=O)O)cc2=O)o1 ; Nc1ncc(C#CC2CC2)s1 ; Cc1nnc(Cn2cc(-c3cc(C(F)F)ccc3Cl)c(C(=O)Nc3ncc(C#CC4CC4)s3)cc2=O)o1]

### Step 2/5-(2-chloro-5-(difluoromethyl)phenyl)-1-((5-methyl-1,3,4-oxadiazol-2-yl)methyl)-2-oxo-1,2-dihydropyridine-4-carboxylic acid

To methyl 5-(2-chloro-5-(difluoromethyl)phenyl)-1-((5-methyl-1,3,4-oxadiazol-2-yl)methyl)-2-oxo-1,2-dihydropyridine-4-carboxylate (504 mg, 1.23 mmol) in MeOH (0.5 mL), 1,4-dioxane (2.0 mL) and H₂O (0.5 mL) was added LiOH·H₂O (105 mg, 2.50 mmol) and the resulting mixture was stirred at 60 °C for 1.5 h. The reaction mixture was evaporated in vacuo, acidified with formic acid (200 μL, 5.30 mmol,) and extracted with EtOAc. The organic layer was separated, concentrated in vacuo to give 5-(2-chloro-5-(difluoromethyl)phenyl)-1-((5-methyl-1,3,4-oxadiazol-2-yl)methyl)-2-oxo-1,2-dihydropyridine-4-carboxylic acid (298 mg, 61% yield) and used directly without purification in the next step. LCMS n/z 396.3 [M+H]⁺.

### Step 3/Compound 114

A mixture of 5-(2-chloro-5-(difluoromethyl)phenyl)-1-((5-methyl-1,3,4-oxadiazol-2-yl)methyl)-2-oxo-1,2-dihydropyridine-4-carboxylic acid (298 mg, 753 μmol), Intermediate 18 (125 mg, 761 μmol), EDC (290 mg, 1.51 mmol) in pyridine (1.5 mL) was heated for 15 min at 50 °C. The reaction mixture was concentrated in vacuo, dissolved in DMSO (1 mL) and purified on reverse phase preparative HPLC eluting with CH₃CN (50-80%) in water both containing 0.1% FA. The relevant fractions were combined and lyophilized to give Compound 114 (120 mg, 29% yield). ¹H NMR (400 MHz, DMSO-d₆) δ 13.08 (s, 1H), 7.98 (s, 1H), 7.63-7.51 (m, 4H), 7.05 (t, J=55.6 Hz, 1H), 6.87 (s, 1H), 5.38 (s, 2H), 3.29 (s, 3H), 1.52 (t, J=8.2, 5.0 Hz, 1H), 0.89-0.79 (m, 2H), 0.72-0.63 (m, 2H). LCMS m/z 542.0 [M+H]⁺.

### Compound 115/Method E/5-(2-chloro-5-(trifluoromethyl)phenyl)-N-(5-(cyclopropylethynyl)thiazol-2-yl)-1-((5-methyl-1,3,4-oxadiazol-2-yl)methyl)-2-oxo-1,2-dihydropyridine-4-carboxamide

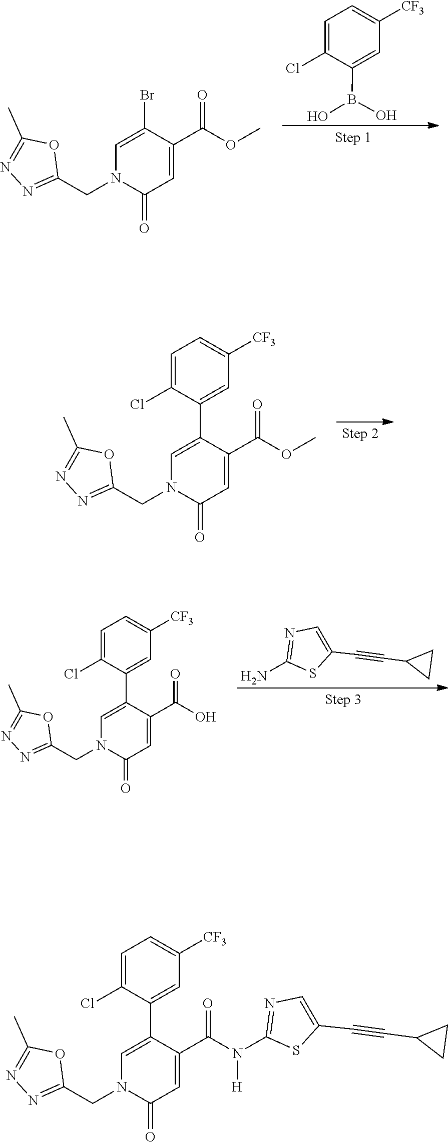 [SMILES: COC(=O)c1cc(=O)n(Cc2nnc(C)o2)cc1Br ; OB(O)c1cc(C(F)(F)F)ccc1Cl ; COC(=O)c1cc(=O)n(Cc2nnc(C)o2)cc1-c1cc(C(F)(F)F)ccc1Cl ; Cc1nnc(Cn2cc(-c3cc(C(F)(F)F)ccc3Cl)c(C(=O)O)cc2=O)o1 ; Nc1ncc(C#CC2CC2)s1 ; Cc1nnc(Cn2cc(-c3cc(C(F)(F)F)ccc3Cl)c(C(=O)Nc3ncc(C#CC4CC4)s3)cc2=O)o1]

### Step 2/5-(2-chloro-5-(trifluoromethyl)phenyl)-1-((5-methyl-1,3,4-oxadiazol-2-yl)methyl)-2-oxo-1,2-dihydropyridine-4-carboxylic acid

A mixture of methyl 5-(2-chloro-5-(trifluoromethyl)phenyl)-1-((5-methyl-1,3,4-oxadiazol-2-yl)methyl)-2-oxo-1,2-dihydropyridine-4-carboxylate (2.14 g, 88% pure, 4.40 mmol), LiOH·H₂O (421 mg, 10.0 mmol) in MeOH (5 mL), 1,4-dioxane (20 mL) and H₂O (5 mL) was stirred at 60 °C for 1 h. The reaction mixture was evaporated in vacuo, diluted with water (100 mL), cooled to 0 °C and acidified with Formic acid (772 μL, 20.5 mmol). The resulting precipitate was filtered and dissolved in EtOAc. The organic phase was dried over MgSO₄, concentrated in vacuo to afford 5-(2-chloro-5-(trifluoromethyl)phenyl)-1-((5-methyl-1,3,4-oxadiazol-2-yl)methyl)-2-oxo-1,2-dihydropyridine-4-carboxylic acid (2.07 g, 75% pure by HPLC). LCMS m/z 414.2 [M+H]⁺.

### Step 3/Compound 115

A mixture of 5-(2-chloro-5-(trifluoromethyl)phenyl)-1-((5-methyl-1,3,4-oxadiazol-2-yl)methyl)-2-oxo-1,2-dihydropyridine-4-carboxylic acid (1.02 g, 75% pure by HPLC, 1.85 mmol), Intermediate 18 (405 mg, 2.47 mmol, EDC (1.42 g, 7.40 mmol) in pyridine (8 mL) was stirred overnight at rt. Additional EDC (4.26 g, 22.2 mmol) was added and the resulting mixture was stirred overnight at rt. The reaction mixture was concentrated in vacuo, water was added to the residue, stirred and the precipitate was filtered. The latter was dissolved in DCM, purified on silica gel chromatography eluting a gradient of Acetone (0-80%) in DCM. The relevant fractions were concentrated in vacuo, dissolved in acetonitrile/water 1:1 and finally lyophilized to give Compound 115 (246 mg, 24% yield). ¹H NMR (400 MHz, DMSO-d₆) δ 13.09 (s, 1H), 8.03 (s, 1H), 7.82-7.68 (m, 2H), 7.66 (d, J=8.2 Hz, 1H), 7.57 (s, 1H), 6.89 (s, 1H), 5.37 (s, 2H), 3.26 (s, 3H), 1.52 (tt, J=8.2, 5.0 Hz, 1H), 0.93-0.77 (m, 2H), 0.77-0.57 (m, 2H). LCMS m/z 560.0 [M+1]⁺.

### Compound 117/Method D/N-(5-(cyclopropylethynyl)thiazol-2-yl)-2′-(difluoromethyl)-5′-methoxy-6-(1-methyl-1H-pyrazol-3-yl)-[4,4′-bipyridine]-3-carboxamide

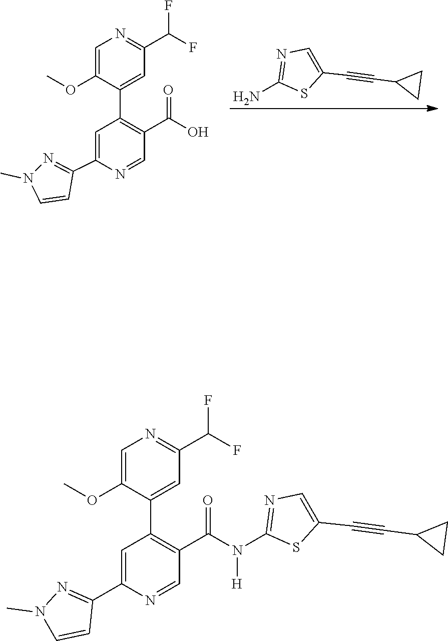 [SMILES: COc1cnc(C(F)F)cc1-c1cc(-c2ccn(C)n2)ncc1C(=O)O ; Nc1ncc(C#CC2CC2)s1 ; COc1cnc(C(F)F)cc1-c1cc(-c2ccn(C)n2)ncc1C(=O)Nc1ncc(C#CC2CC2)s1]

A mixture of 2-(2-(difluoromethyl)-5-methoxypyridin-4-yl)-4-(1-methyl-1H-pyrazol-3-yl)benzoic acid (76.0 mg, 211 μmol) (see Compound 113, Step 2), Intermediate 18 (66.0 mg, 402 μmol) and EDC (97 mg, 506 μmol) was stirred in pyridine (1.5 mL) for 2 h 20 min. The resulting mixture was concentrated in vacuo, dissolved in DMSO and purified by preparative HPLC eluting with a gradient of CH₃CN (30 to 60%) in water containing 10 mM ammonium bicarbonate (pH adjusted to 10 with NH₄OH). Appropriate fractions were combined and lyophilized to afford Compound 117 (47.0 mg, 44% yield) as an off white fluffy solid. ¹H NMR (400 MHz, DMSO-d₆) δ 13.02 (s, 1H), 8.91 (s, 1H), 8.47 (s, 1H), 7.92 (s, 1H), 7.86 (d, J=2.3 Hz, 1H), 7.77 (s, 1H), 7.64 (s, 1H), 7.01 (t, J=55.0 Hz, 1H), 6.95 (d, J=2.2 Hz, 1H), 3.94 (s, 3H), 3.67 (s, 3H), 1.58 (tt, J=8.2, 5.0 Hz, 1H), 0.89 (dt, J=8.1, 3.2 Hz, 2H), 0.74 (dt, J=4.9, 3.2 Hz, 2H). ¹⁹F NMR (376 MHz, DMSO-d₆) δ −113.31 (d, J=55.2 Hz). 220 nm). LCMS m/z 507.2 [M]⁺.

### Compound 118/Method D/N-(5-(cyclopropylethynyl)-1,3,4-thiadiazol-2-yl)-2-(2-(difluoromethyl)-5-methoxypyridin-4-yl)-4-(methyl(5-methyl-1,3,4-oxadiazol-2-yl)amino)benzamide

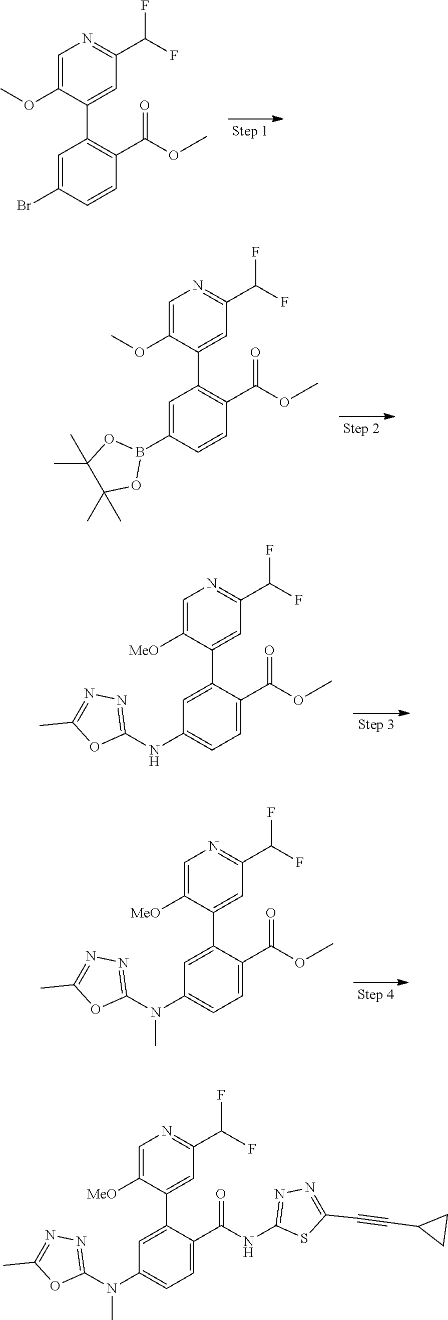

### Step 1/methyl 2-(2-(difluoromethyl)-5-methoxypyridin-4-yl)-4-(4,4,5,5-tetramethyl-1,3,2-dioxaborolan-2-yl)benzoate

To a stirred solution of Intermediate 20 (1.00 g, 2.69 mmol) in Dioxane (15 ml) were added Bis(pinacolato)diboron (1.71 g, 6.73 mmol) and KOAc (0.790 g, 8.07 mmol). The reaction mixture was purged with argon gas for 15 min followed by addition of PdCl₂(dppf)·DCM (0.220 g, 0.269 mmol). The reaction mixture was stirred at 100 °C for 4 h. The reaction mixture was poured into water and extracted with EtOAc (3×25 mL), the combined organic layers were collected, dried over anhydrous Na₂SO₄, filtered, and concentrated in vacuo. The residue was purified by silica gel chromatography using a gradient of EtOAc (0 to 50%) in Hexanes. Appropriate fractions were combined and concentrated in vacuo to afford methyl 2-(2-(difluoromethyl)-5-methoxypyridin-4-yl)-4-(4,4,5,5-tetramethyl-1,3,2-dioxaborolan-2-yl)benzoate (0.60 g, 53%). ¹H NMR (400 MHz, DMSO d₆) δ 8.46 (s, 1H), 7.89-7.83 (m, 2H), 7.60 (s, 1H), 7.57 (s, 1H), 6.97 (t, J=54.8 Hz, 1H), 3.82 (s, 3H), 3.658 (s, 3H), 1.31 (s, 12H). LCMS m/z 419.2 [M+H]⁺.

### Step 2/methyl 2-(2-(difluoromethyl)-5-methoxypyridin-4-yl)-4-((5-methyl-1,3,4-oxadiazol-2-yl)amino)benzoate

To a stirred solution of methyl 2-(2-(difluoromethyl)-5-methoxypyridin-4-yl)-4-(4,4,5,5-tetramethyl-1,3,2-dioxaborolan-2-yl)benzoate (1.20 g, 2.86 mmol) and 5-methyl-1,3,4-oxadiazol-2-amine (0.280 g, 2.86 mmol) in CH₃CN:EtOH (5:1; 12 mL) were added Et₃N (190 μL, 1.43 mmol), Cu(OAc)₂ (0.510 g, 2.86 mmol) and 4 Å powdered molecular sieves (200 mg). The reaction mixture was stirred at rt for 12 hrs under air. The reaction mixture was filtered through a Celite bed and washed by EtOAc (3×50 mL). The combined organic layer was collected, dried over Na₂SO₄, filtered, and concentrated in vacuo. The residue was purified by silica gel chromatography using a gradient of EtOAc (0 to 50%) in Hexanes. Appropriate fractions were combined and concentrated in vacuo to afford methyl 2-(2-(difluoromethyl)-5-methoxypyridin-4-yl)-4-((5-methyl-1,3,4-oxadiazol-2-yl)amino)benzoate (100 mg, 9%). ¹H NMR (400 MHz, DMSO d₆) δ 10.88 (s, 1H), 8.48 (s, 1H), 7.93 (d, J=8.4 Hz, 1H), 7.71 (d, J=6.8 Hz, 1H), 7.52 (s, 2H), 6.98 (t, J=55.2 Hz, 1H), 3.85 (s, 3H), 3.61 (s, 3H), 2.43 (s, 3H). LCMS m/z 391.4 [M+H]⁺.

### Step 3/methyl 2-(2-(difluoromethyl)-5-methoxypyridin-4-yl)-4-(methyl(5-methyl-1,3,4-oxadiazol-2-yl)amino)benzoate

A solution of methyl 2-(2-(difluoromethyl)-5-methoxypyridin-4-yl)-4-((5-methyl-1,3,4-oxadiazol-2-yl)amino)benzoate (100 mg, 0.250 mmol) in DMF (1.0 ml) was cooled to 0 °C and NaH (60% in oil) (100 mg, 2.56 mmol) was added. The reaction mixture was stirred at 0 °C for 30 min followed by addition of CH₃I (19 μL, 0.30 mmol). Reaction mixture was stirred at room temperature for 2 hrs. The resulting mixture was poured into water and extracted with EtOAc (3×10 mL), the combined organic layers were collected, dried over anhydrous Na₂SO₄, filtered, and concentrated in vacuo to afford methyl 2-(2-(difluoromethyl)-5-methoxypyridin-4-yl)-4-(methyl(5-methyl-1,3,4-oxadiazol-2-yl)amino)benzoate (0.09 g, 87%) that was used as such for the next step without purification. LCMS m/z 404.7 [M+H]⁺.

### Step 4/Compound 118

To a stirred solution of methyl 2-(2-(difluoromethyl)-5-methoxypyridin-4-yl)-4-(methyl(5-methyl-1,3,4-oxadiazol-2-yl)amino)benzoate (50.0 mg, 0.120 mmol) and Intermediate 9 (20.0 mg, 0.120 mmol) In THF (0.5 mL) was added 1,3,4,6,7,8-hexahydro-2H-pyrimido[1,2-a]pyrimidine (80.0 mg, 0.61 mmol) at rt. The reaction mixture was stirred at 70 °C for 3 h. The reaction mixture was poured into water and extracted with EtOAc (3×10 mL), the combined organic layers were collected, dried over anhydrous Na₂SO₄, filtered, and concentrated in vacuo. The residue was purified by silica gel chromatography using a gradient (0 to 3%) of MeOH in DCM. Product was further purified using preparatibe HPLC purification to get pure Compound 118 (10.0 mg, 15%). ¹H NMR (400 MHz, DMSO d₆) δ 13.21 (s, 1H), 8.38 (s, 1H), 7.85 (d, J=8.8 Hz, 1H), 7.71 (d, J=9.2 Hz, 1H), 7.65 (s, 1H), 7.59 (s, 1H), 6.97 (t, J=55.2 Hz, 1H), 3.63 (s, 3H), 3.55 (s, 3H), 2.40 (s, 3H), 1.69 (s, 1H), 0.99 (d, J=5.2, 2H), 0.86 (s, 2H). LCMS m/z 538.3 [M+HP.

### Compound 122/Method D/N-(5-(cyclopropylethynyl)-1,3,4-thiadiazol-2-yl)-2″-(difluoromethyl)-5″-methoxy-2-oxo-2H-[1,2′:4′,4″-terpyridine]-5′-carboxamide

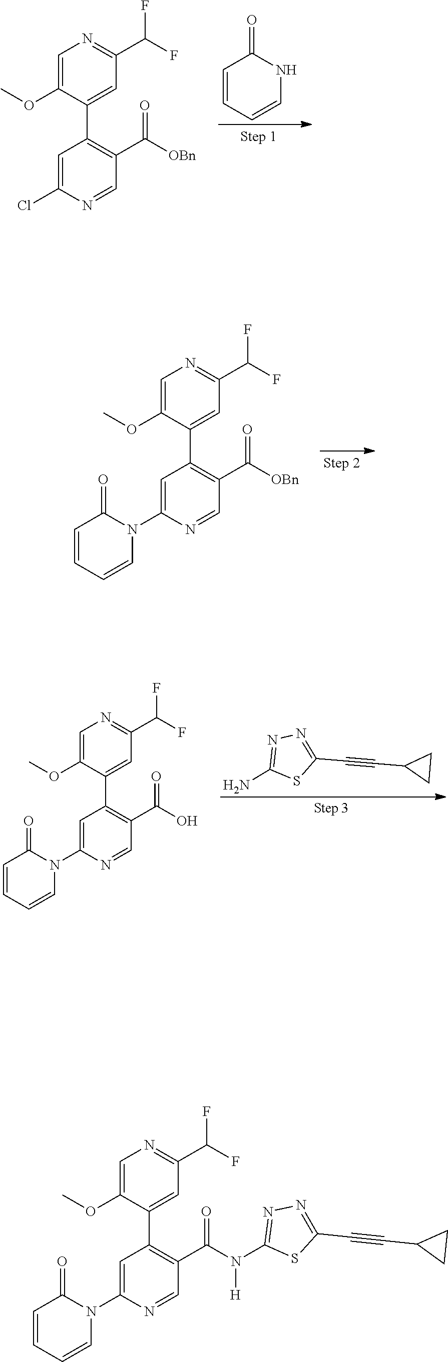 [SMILES: COc1cnc(C(F)F)cc1-c1cc(Cl)ncc1C(=O)OCc1ccccc1 ; O=c1cccc[nH]1 ; COc1cnc(C(F)F)cc1-c1cc(-n2ccccc2=O)ncc1C(=O)OCc1ccccc1 ; COc1cnc(C(F)F)cc1-c1cc(-n2ccccc2=O)ncc1C(=O)O ; Nc1nnc(C#CC2CC2)s1 ; COc1cnc(C(F)F)cc1-c1cc(-n2ccccc2=O)ncc1C(=O)Nc1nnc(C#CC2CC2)s1]

### Step 1/benzyl 2″-(difluoromethyl)-5″-methoxy-2-oxo-2H-[1,2′:4′,4″-terpyridine]-5′-carboxylate

To a solution Intermediate 25 (200 mg, 494 μmol) in DMSO (3 mL) were added Copper(I) Iodide (10.0 mg, 52.5 μmol), 1H-pyridin-2-one (56 mg, 589 μmol), 8-Hydroxyquinoline (8.0 mg, 55 μmol) and K₂CO₃ (130 mg, 941 μmol). The mixture was degassed in vacuo and then backfilled with N₂ in a sealed vial. The resulting mixture was stirred at 100 °C for 1 h. The crude reaction mixture was filtered, and the filtrate was purified by preparative HPLC eluting with a gradient of CH₃CN (20 to 100%) in water both containing 0.1% formic acid. Appropriate fractions were combined and lyophilized to afford benzyl 2″-difluoromethyl)-5″-methoxy-2-oxo-2H-[1,2′:4′,4″-terpyridine]-5′-carboxylate (138 mg, 60% yield). LCMS m/z 463.9 [M+1]⁺.

### Step 2/2″-(difluoromethyl)-5″-methoxy-2-oxo-2H-[1,2′:4′,4″-terpyridine]-5′-carboxylic acid

To a solution of 2″-difluoromethyl)-5″-methoxy-2-oxo-2H-[1,2′:4′,4″-terpyridine]-5′-carboxylate (138 mg, 298 μmol) in MeOH (6 mL) was added Pd on activated charcoal (10%) (46.0 mg, 43.2 μmol, 10% purity). The mixture was stirred under an atmosphere of H₂ for 4 h. The reaction mixture was filtered on celite, and the filtrate was concentrated to dryness to provide 2″-(difluoromethyl)-5″-methoxy-2-oxo-2H-[1,2′:4′,4″-terpyridine]-5′-carboxylic acid (100 mg, 90% yield) as an off-white solid, which was used directly in the next step without purification. LCMS m/z 371.8 [M−1]⁻.

### Step 3/Compound 122

To a solution of 2″-(difluoromethyl)-5″-methoxy-2-oxo-2H-[1,2′:4′,4″-terpyridine]-5′-carboxylic acid acid (100 mg, 268 μmol) and Intermediate 9 (45.0 mg, 272 μmol) in pyridine (2 mL) was added EDC (120 mg, 626 μmol). The reaction mixture was stirred at rt for 18 hrs. The volatiles were removed in vacuo. The residue was dissolved in DMSO, filtered and the filtrate was purified by preparative HPLC eluting with a gradient of CH₃CN (25 to 100%) in water both containing 0.1% formic acid. Appropriate fractions were combined and lyophilized to afford Compound 122 (44 mg, 32% yield) as an off-white solid. ¹H NMR (400 MHz, DMSO-d₆) δ 13.57 (s, 1H), 8.95 (s, 1H), 8.44 (s, 1H), 7.99 (s, 1H), 7.95 (m, 1H), 7.70 (s, 1H), 7.54 (m, 1H), 6.95 (t, J=55.1 Hz, 1H), 6.51 (d, J=9.2 Hz, 1H), 6.40 (d, J=6.8 Hz, 1H), 3.63 (s, 3H), 1.66 (m, 1H), 1.06-0.91 (m, 2H), 0.90-0.77 (m, 2H). LCMS m/z 520.8 [M+H]⁺.

### Compound 124/Method D/N-(5-(cyclopropylethynyl)-1,3,4-thiadiazol-2-yl)-2-(2-(difluoromethyl)-5-methoxypyridin-4-yl)-4-(1-(methylsulfonyl)methyl)-1H-pyrazol-3-yl)benzamide

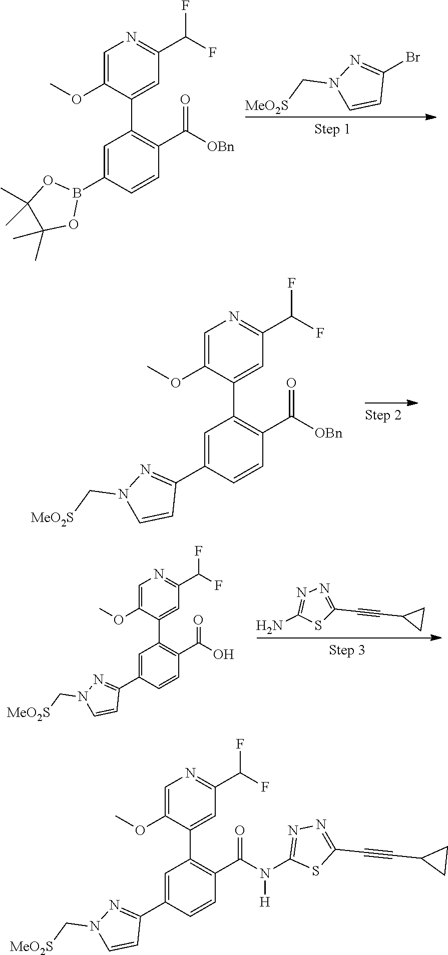 [SMILES: COc1cnc(C(F)F)cc1-c1cc(B2OC(C)(C)C(C)(C)O2)ccc1C(=O)OCc1ccccc1 ; CS(=O)(=O)Cn1ccc(Br)n1 ; COc1cnc(C(F)F)cc1-c1cc(-c2ccn(CS(C)(=O)=O)n2)ccc1C(=O)OCc1ccccc1 ; COc1cnc(C(F)F)cc1-c1cc(-c2ccn(CS(C)(=O)=O)n2)ccc1C(=O)O ; Nc1nnc(C#CC2CC2)s1 ; COc1cnc(C(F)F)cc1-c1cc(-c2ccn(CS(C)(=O)=O)n2)ccc1C(=O)Nc1nnc(C#CC2CC2)s1]

### Step 1/benzyl 2-(2-(difluoromethyl)-5-methoxypyridin-4-yl)-4-(1-((methylsulfonyl)methyl)-1H-pyrazol-3-yl)benzoate

A microwave sealed vial was charged with Intermediate 35 (85.0 mg, 172 μmol), 3-bromo-1-(methylsulfonylmethyl)pyrazole (50.0 mg, 209 μmol), Na₂CO₃ (2 M, 200 μL, 400 mmol) and Pd(dppf)Cl₂·DCM (12.0 mg, 16.4 μmol) in dioxane (2 mL). The reaction mixture was stirred under N₂ at 80 °C for 1 h. The reaction mixture was diluted with water (30 mL), extracted with EtOAc (3×30 ml). The combined organic extracts were washed with brine, dried over Na₂SO₄, filtered and concentrated. The residue was purified by silica gel chromatography eluting with a gradient of EtOAc (0 to 70%) in heptane to provide benzyl 2-(2-(difluoromethyl)-5-methoxypyridin-4-yl)-4-(1-((methylsulfonyl)methyl)-1H-pyrazol-3-yl)benzoate (63 mg, 70% yield). LCMS m/z 527.8 [M+H]⁺.

### Step 2/2-(2-(difluoromethyl)-5-methoxypyridin-4-yl)-4-(1-((methylsulfonyl)methyl)-1H-pyrazol-3-yl)benzoic acid

To a solution of benzyl 2-(2-(difluoromethyl)-5-methoxypyridin-4-yl)-4-(1-((methylsulfonyl)methyl)-1H-pyrazol-3-yl)benzoate (63.0 mg, 119 μmol) In MeOH (3 mL) was added Pd 10% on activated charcoal (20.0 mg, 18.8 μmol, 10% purity). The mixture was stirred under an atmosphere of H₂ at rt for 18 h. The reaction mixture was filtered and the filtrate was concentrated to dryness to provide 2-(2-(difluoromethyl)-5-methoxypyridin-4-yl)-4-(1-((methylsulfonyl)methyl)-1H-pyrazol-3-yl)benzoic acid (52.0 mg, 100% yield) as an off-white solid which was used in the next step without further purification. LCMS m/z 435.7 [M−H]⁻.

### Step 3/Compound 124

To a solution of 2-(2-(difluoromethyl)-5-methoxypyridin-4-yl)-4-(1-((methylsulfonyl)methyl)-1H-pyrazol-3-yl)benzoic acid (52.0 mg, 119 μmol) and Intermediate 9 (22.0 mg, 133 μmol) in pyridine (1 mL) was added EDC (50.0 mg, 261 μmol). The reaction was stirred at rt for 18 hrs. The crude reaction mixture was concentrated to dryness in vacuo. The residue was purified by preparative HPLC eluting with a gradient of CH₃CN (25 to 100%) in water both containing 0.1% formic acid. Appropriate fractions were combined and lyophilized to afford Compound 124 (40 mg, 58% yield) as an off-white solid. ¹H NMR (400 MHz, DMSO-d₆) δ 13.24 (s, 1H), 8.35 (s, 1H), 8.01 (d, J=8.0, 1H), 7.94 (d, J=2.5 Hz, 1H), 7.91-7.78 (m, 2H), 7.65 (s, 1H), 7.17-6.77 (m, 2H), 5.78 (s, 2H), 3.59 (s, 3H), 3.04 (s, 3H), 1.65 (m, 1H), 0.97-0.88 (m, 2H), 0.85-0.76 (m, 2H). LCMS m/z 584.7 [M+H]⁺.

### Compound 130/Method F/N-(5-(cyclopropylethynyl)-1,3,4-thiadiazol-2-yl)-2′-(difluoromethyl)-6-(6-hydroxy-6-methyl-2-azaspiro[3.3]heptan-2-yl)-5′-methoxy-[4,4′-bipyridine]-3-carboxamide

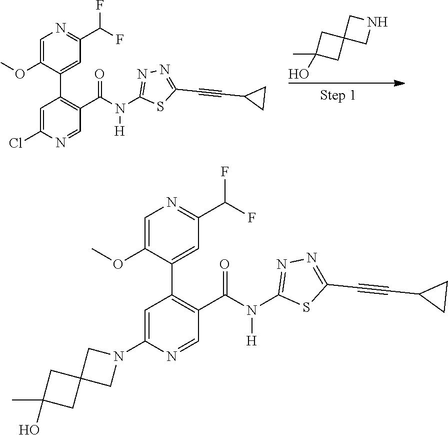

A solution of Intermediate 23 (50 mg, 108 μmol), DIPEA (189 μL, 1.08 mmol,) and 6-methyl-2-azaspiro[3.3]heptan-6-ol (138 mg, 1.08 mmol) and in DMF (810 μL) was stirred at 130 °C for 10 min. After cooling to rt, the mixture was filtered, and the filtrate was purified by preparative HPLC eluting with a gradient of CH₃CN (20 to 80%) in water both containing 0.1% formic acid. Appropriate fractions were combined and lyophilized to afford Compound 23 (8.0 mg, 13% yield, 94%) as a white solid. ¹H NMR (400 MHz, DMSO-d₆) δ 12.91 (s, 1H), 8.47 (s, 1H), 8.34 (s, 1H), 7.58 (s, 1H), 6.92 (t, J=55.1, 1H), 6.27 (s, 1H), 4.90 (s, 1H), 4.03 (d, J=26.6 Hz, 4H), 3.59 (s, 3H), 2.23-2.14 (m, 4H), 1.63 (m, 1H), 1.15 (s, 3H), 0.93 (m, 2H), 0.80 (m, 2H). LCMS m/z 553.1 [M+H]⁺.

### Compound 133/Method D/N-(5-(cyclopropylethynyl)-1,3,4-thiadiazol-2-yl)-2-(2-(difluoromethyl)-5-methoxypyridin-4-yl)-4-(4-oxo-6,7-dihydropyrazolo[1,5-a]pyrazin-5(4H)-yl)benzamide

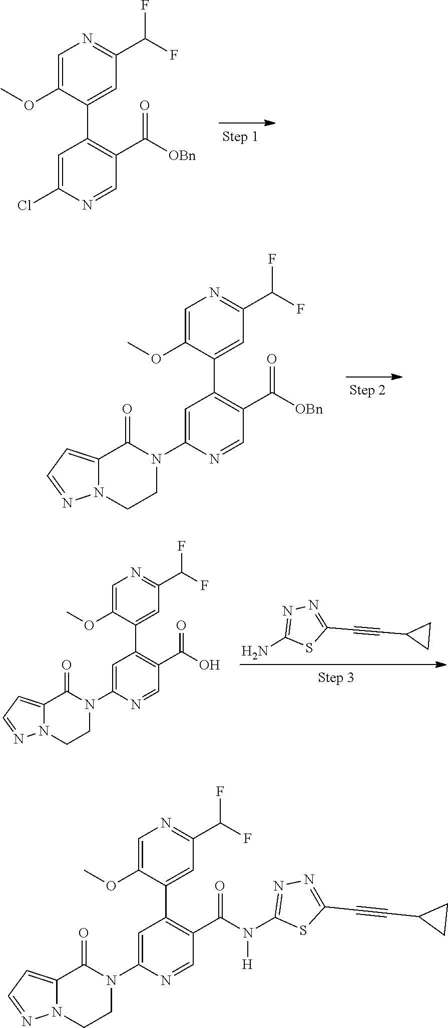 [SMILES: COc1cnc(C(F)F)cc1-c1cc(Cl)ncc1C(=O)OCc1ccccc1 ; COc1cnc(C(F)F)cc1-c1cc(N2CCn3nccc3C2=O)ncc1C(=O)OCc1ccccc1 ; COc1cnc(C(F)F)cc1-c1cc(N2CCn3nccc3C2=O)ncc1C(=O)O ; Nc1nnc(C#CC2CC2)s1 ; COc1cnc(C(F)F)cc1-c1cc(N2CCn3nccc3C2=O)ncc1C(=O)Nc1nnc(C#CC2CC2)s1]

### Step 1/benzyl 2′-(difluoromethyl)-5′-methoxy-6-(4-oxo-6,7-dihydropyrazolo[1,5-a]pyrazin-5(4H)-yl)-[4,4′ bipyridine]-3-carboxylate

To a solution of Intermediate 25 (250 mg, 618 μmol), 6,7-dihydro-5H-pyrazolo[1,5-a]pyrazin-4-one (100 mg, 729 μmol) and Xantphos Pd G3 (55.0 mg, 57.9 μmol) in dioxane (3.5 mL) was added Cs₃CO₃ (400 mg, 1.23 mmol). The vessel was flushed with N₂, sealed, and stirred at 80 °C for 2 h. The cooled reaction mixture was diluted with DCM and then adsorbed onto silica. The residue was purified by silica gel chromatography eluting with a gradient of EtOAc (15 to 100%) in heptane to provide benzyl 2′-(difluoromethyl)-5′-methoxy-6-(4-oxo-6,7-dihydropyrazolo[1,5-a]pyrazin-5(4H)-yl)-[4,4′-bipyridine]-3-carboxylate (272 mg, 87% yield). LCMS m/z 505.8 [M+H]+.

### Step 2/2′-(difluoromethyl)-5′-methoxy-6-(4-oxo-6,7-dihydropyrazolo[1,5-a]pyrazin-5(4H)-yl)-[4,4′-bipyridine]-3-carboxylic acid

Benzyl 2′-(difluoromethyl)-5′-methoxy-6-(4-oxo-6,7-dihydropyrazolo[1,5-a]pyrazin-5(4H)-yl)-[4,4′-bipyridine]-3-carboxylate (272 mg, 538 μmol) was dissolved in EtOH (4 mL) and treated with Pd (10%) on Carbon (27 mg, 253 μmol). The mixture was stirred at rt under an atmosphere of hydrogen overnight. The reaction was not complete, and additional Pd (10%) on Carbon (27 mg, 253.71 μmol) was added and the mixture was stirred at rt under an atmosphere of hydrogen overnight again. The reaction mixture was filtered through Celite and concentrated to give 2′-(difluoromethyl)-5′-methoxy-6-(4-oxo-6,7-dihydropyrazolo[1,5-a]pyrazin-5(4H)-yl)-[4,4′-bipyridine]-3-carboxylic acid (220 mg, 98% yield) which was used in the next step without further purification. LCMS m/z 415.8 [M+H]⁺.

### Step 3/Compound 133

2′-(Difluoromethyl)-5′-methoxy-6-(4-oxo-6,7-dihydropyrazolo[1,5-a]pyrazin-5(4H)-yl)-[4,4′-bipyridine]-3-carboxylic acid (110 mg, 265 μmol) and Intermediate 9 (65.0 mg, 393 μmol) were dissolved in pyridine (2 mL). EDC (125 mg, 652 μmol) was added and the mixture was stirred at rt over the weekend. The crude reaction mixture was filtered, and the filtrate was purified by preparative HPLC eluting with a gradient of CH₃CN (40 to 70%) in water both containing 0.1% formic acid. Appropriate fractions were combined and lyophilized to afford Compound 133 (94.0 mg, 63% yield). ¹H NMR (DMSO-d₆) δ: 13.49 (s, 1H), 8.90 (s, 1H), 8.47 (s, 1H), 8.08 (s, 1H), 7.68 (d, J=2.0 Hz, 1H), 7.66 (s, 1H), 7.01 (t, J=55.1 Hz, 1H), 6.97 (d, J=2.1 Hz, 1H), 4.66-4.51 (m, 4H), 3.67 (s, 3H), 1.70 (tt, J=8.2, 5.0 Hz, 1H), 0.99 (dt, J=8.2, 3.2 Hz, 2H), 0.90-0.84 (m, 2H). LCMS m/z 562.7 [M+H]⁺.

### Compound 139/Method D/N-(5-(cyclopropylethynyl)thiazol-2-yl)-2′-(difluoromethyl)-5′-methoxy-5-(1-methyl-1H-pyrazol-3-yl)-[3,4′-bipyridine]-2-carboxamide

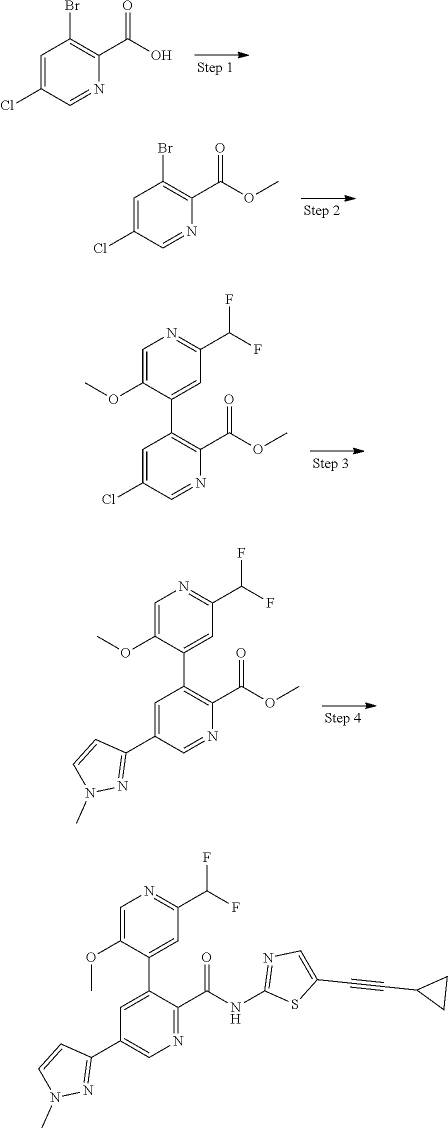

### Step 1/methyl 3-bromo-5-chloropicolinate

3-Bromo-5-chloropicolinic acid (1.00 g, 4.23 mmol) was dissolved in MeOH (10 mL) at rt followed by dropwise addition of H₂SO₄ (1.0 mL). The reaction mixture was stirred at 70 °C for 4 h. The reaction mixture was concentrated in vacuo and the residue was quenched in aqueous solution of sodium bicarbonate (100 mL). Solids were filtered out, washed with hexanes (100 mL) and dried to afford methyl 3-bromo-5-chloropicolinate (0.95 g, 89%). LCMS m/z 251.9 [M+H]*.

### Step 2/methyl 5-chloro-2′-(difluoromethyl)-5′-methoxy-[3,4′-bipyridine]-2-carboxylate

To a mixture of methyl 3-bromo-5-chloropicolinate (0.900 g, 3.59 mmol) and Intermediate 19 (1.02 g, 3.59 mmol) in dioxane:water (8:2; 9.0 mL) at rt was added K₂CO₃ (0.990 g, 7.18 mmol). The reaction mixture was degassed with argon for 5 min followed by addition of PdCl₂(dppf)·DCM (0.290 g, 0.36 mmol). The reaction mixture was heated at 70 °C for 1 h. The resulting mixture was poured into water and extracted with EtOAc (3×30 mL), the combined organic layer was dried over anhydrous Na₂SO₄, filtered, and concentrated in vacuo. The residue was purified by silica gel chromatography using a gradient of EtOAc (0 to 50%) in Hexanes. Appropriate fractions were combined and concentrated in vacuo to afford methyl 5-chloro-2′-(difluoromethyl)-5′-methoxy-[3,4′-bipyridine]-2-carboxylate (0.90 g, 76.2%). LCMS m/z 329.0 [M+H]⁺.

### Step 3/methyl 2′-(difluoromethyl)-5′-methoxy-5-(1-methyl-1H-pyrazol-3-yl)-[3,4′-bipyridine]-2-carboxylate

To a mixture of Methyl 5-chloro-2′-(difluoromethyl)-5′-methoxy-[3,4′-bipyridine]-2-carboxylate (0.500 g, 1.52 mmol) and 1-methyl-3-(4,4,5,5-tetramethyl-1,3,2-dioxaborolan-2-yl)-1H-pyrazole (0.316 g, 1.52 mmol) in dioxane-water (8:2; 5.0 mL) at room temperature was added K₂CO₃ (0.42 g, 3.04 mmol). The reaction mixture was degassed with argon for 5 min followed by addition of PdCl₂(dppf)·DCM (0.12 g, 0.15 mmol). Then the reaction mixture was heated at 70 °C for 1 h. The reaction mixture was poured into water and extracted with EtOAc (3×25 mL), the combined organic layer was collected, dried over anhydrous Na₂SO₄, filtered, and concentrated in vacuo. The residue was purified by silica gel chromatography using a gradient of EtOAc (0 to 80%) in Hexanes. Appropriate fractions were combined and concentrated in vacuo to afford methyl 2′-(difluoromethyl)-5′-methoxy-5-(1-methyl-1H-pyrazol-3-yl)-[3,4′-bipyridine]-2-carboxylate (0.40 g, 70%). LCMS m/z 375.2 [M+H]⁺.

### Step 4/Compound 139

Methyl 2′-(difluoromethyl)-5′-methoxy-5-(1-methyl-1H-pyrazol-3-yl)-[3,4′-bipyridine]-2-carboxylate (0.15 g, 0.40 mmol) and Intermediate 18 (0.066 g, 0.40 mmol) were dissolved in THF (1.5 mL) followed by addition of 1,3,4,6,7,8-hexahydro-2H-pyrimido[1,2-a]pyrimidine (0.27 g, 2.00 mmol). The reaction mixture was stirred at room temperature for 5 h. The reaction mixture was quenched with water (10 mL) and extracted with EtOAc (3×10 mL), then combined organic layer was dried over Na₂SO₄, filtered, and concentrated in vacuo. The residue was purified by silica gel chromatography using a gradient of MeOH (0 to 80%) in DCM. Appropriate fractions were combined and concentrated in vacuo to afford Compound 139 (45 mg, 22% yield). ¹H NMR (400 MHz, DMSO d₆) δ 10.48 (s, 1H), 9.19 (s, 1H), 8.51 (s, 1H), 8.27 (s, 1H), 7.88 (s, 1H), 7.78 (s, 1H), 7.67 (s, 1H), 7.07 (s, 1H), 7.02 (t, J=55.2 Hz, 1H), 3.97 (s, 3H), 3.74 (s, 3H), 1.59 (m, 1H), 0.93-0.90 (m, 2H), 0.75 (d, J=2.4, 2H). LCMS m/z 507.0 [M+H]⁺.

### Compound 140/Method D N-(5-(cyclopropylethynyl)-1,3,4-thiadiazol-2-yl)-3-(2-(difluoromethyl)-5-methoxypyridin-4-yl)-5-(1-methyl-1H-pyrazol-3-yl)pyrazine-2-carboxamide

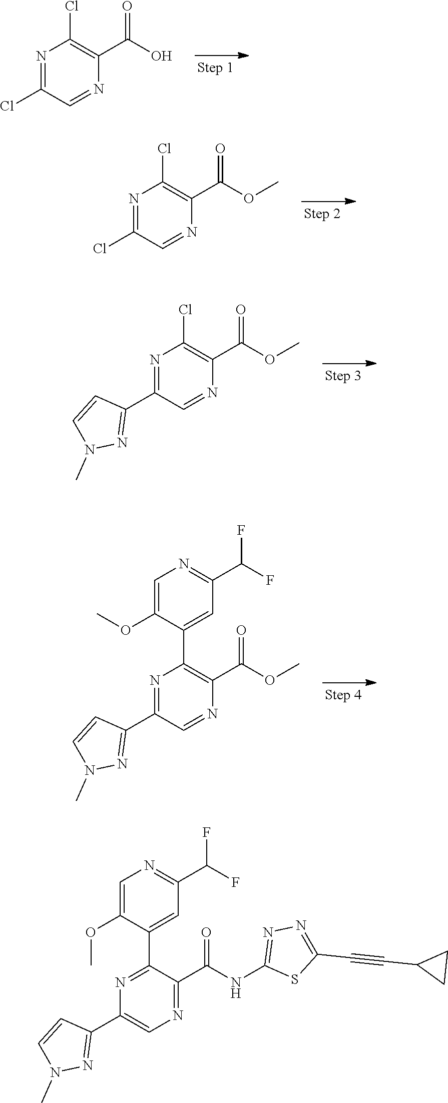 [SMILES: O=C(O)c1ncc(Cl)nc1Cl ; COC(=O)c1ncc(Cl)nc1Cl ; COC(=O)c1ncc(-c2ccn(C)n2)nc1Cl ; COC(=O)c1ncc(-c2ccn(C)n2)nc1-c1cc(C(F)F)ncc1OC ; COc1cnc(C(F)F)cc1-c1nc(-c2ccn(C)n2)cnc1C(=O)Nc1nnc(C#CC2CC2)s1]

### Step 1/methyl 3,5-dichloropyrazine-2-carboxylate

To a stirred solution of 3,5-dichloropyrazine-2-carboxylic acid (7.00 g, 36.3 mmol) in DMF (70 mL) was added NaHCO₃ (3.66 g, 43.5 mmol) followed by addition of CH₃a (13.5 mL, 217.6 mmol). The reaction mixture was stirred at rt for 16 h. The reaction mixture was quenched with water and extracted with EtOAc (3×10 mL). The combined organic layers were collected, dried over anhydrous Na₂SO₄, filtered, and concentrated in vacuo. The residue was purified by silica gel chromatography using a gradient of EtOAc (0 to 20%) in Hexanes. Appropriate fractions were combined and concentrated in vacuo to afford methyl 3,5-dichloropyrazine-2-carboxylate (5.30 g, 70% yield). ¹H NMR (400 MHz, DMSO d₆) δ 8.92 (s, 1H), 3.93 (s, 3H).

### Step 2/methyl 3-chloro-5-(1-methyl-1H-pyrazol-3-yl)pyrazine-2-carboxylate

A stirred mixture of methyl 3,5-dichloropyrazine-2-carboxylate (1.00 g, 4.83 mmol) and 1-methyl-3-(4,4,5,5-tetramethyl-1,3,2-dioxaborolan-2-yl)-1H-pyrazole (1.20 g, 5.79 mmol) in Dioxane:Water (3:1) (10 mL) was degassed with N₂ for 10 min. Cs₂CO₃ (3.15 g, 9.66 mmol) was added and the reaction mixture was sonicated under N₂ atmosphere for 5 min followed by addition of PdCl₂(dppf). DCM (394 mg, 0.480 mmol). The reaction mixture was heated at 100 °C for 1 h. The reaction mixture was poured into ice cold water (50 mL) and extracted with EtOAc (3×50 mL), the combined organic layers were combined, dried over anhydrous Na₂SO₄, filtered, and concentrated in vacuo. The residue was purified by silica gel chromatography using a gradient of EtOAc (0 to 40%) in Hexanes. Appropriate fractions were combined and concentrated in vacuo to afford methyl 3-chloro-5-(1-methyl-1H-pyrazol-3-yl)pyrazine-2-carboxylate (600 mg, 49% yield). ¹H NMR (400 MHz, DMSO d₆) δ 9.17 (d, J=1.2 Hz, 1H), 7.95 (s, 1H), 6.98 (dd. J=1.2, 2.0 Hz, 1H), 4.01 (s, 1H), 3.96 (s, 1H). LCMS m/z 253.09 [M+H]⁺.

### Step 3/methyl 3-(2-(difluoromethyl)-5-methoxypyridin-4-yl)-5-(1-methyl-1H-pyrazol-3-yl)pyrazine-2-carboxylate

A mixture of methyl 3-chloro-5-(1-methyl-1H-pyrazol-3-yl)pyrazine-2-carboxylate (0.15 g, 0.59 mmol) and Intermediate 19 (0.18 g, 0.59 mmol) in Dioxane:Water (3:1) was degassed with N₂ for 5 min. K₂CO₃ (0.16 g, 1.19 mmol) was added and the reaction mixture was sonicated under N₂ atmosphere for 5 min followed by addition of PdCl₂(dppf). DCM (48.0 mg, 0.059 mmol). The reaction mixture was heated at 80 °C for 1 h. The reaction mixture was poured onto crushed ice and extracted with EtOAc (3×10 mL), the combined organic layer was collected, dried over anhydrous Na₂SO₄, filtered, and concentrated in vacuo. The residue was purified by silica gel chromatography using a gradient of EtOAc (0 to 70%) in Hexanes. Appropriate fractions were combined and concentrated in vacuo to afford methyl 3-(2-(difluoromethyl)-5-methoxypyridin-4-yl)-5-(1-methyl-1H-pyrazol-3-yl)pyrazine-2-carboxylate (0.13 g, 58% yield). LCMS m/z 375.9 [M+H]⁺.

### Step 4/Compound 140

To a stirred solution of methyl 3-(2-(difluoromethyl)-5-methoxypyridin-4-yl)-5-(1-methyl-1H-pyrazol-3-yl)pyrazine-2-carboxylate (0.13 g, 0.34 mmol) and Intermediate 9 (0.057 g, 0.34 mmol) in THF (1.3 mL) at rt was added 1,3,4,6,7,8-hexahydro-2H-pyrimido[1,2-a]pyrimidine (0.240 g, 1.73 mmol). The reaction mixture was stirred at room temperature for 3 hrs. The reaction mixture was poured into water and extracted with EtOAc (3×10 mL), the combined organic layer was collected, dried over anhydrous Na₂SO₄, filtered, and concentrated in vacuo. The residue was purified by silica gel chromatography using a gradient of MeOH (0 to 1%) in DCM. Appropriate fractions were combined and concentrated in vacuo to afford a residue that was further triturated in diethyl ether and pentane to afford Compound 140 (0.036 g, 20% yield). ¹H NMR (400 MHz, DMSO d₆) δ 13.57 (s, 1H), 9.28 (s, 1H), 8.57 (s, 1H), 7.94 (d, J=3.2 Hz, 2H), 7.22-6.94 (m, 2H), 4.03 (s, 3H), 3.73 (s, 3H), 1.72 (bs, 1H), 1.02 (d, J=5.6 Hz, 2H), 0.91 (bs, 2H). LCMS m/z 509.3 [M+H]⁺.

### Compound 145/Method D/N-(5-(cyclopropylethynyl)-1,3,4-thiadiazol-2-yl)-2′-(difluoromethyl)-5′-methoxy-6-(1H-pyrazol-3-yl)-[4,4′-bipyridine]-3-carboxamide

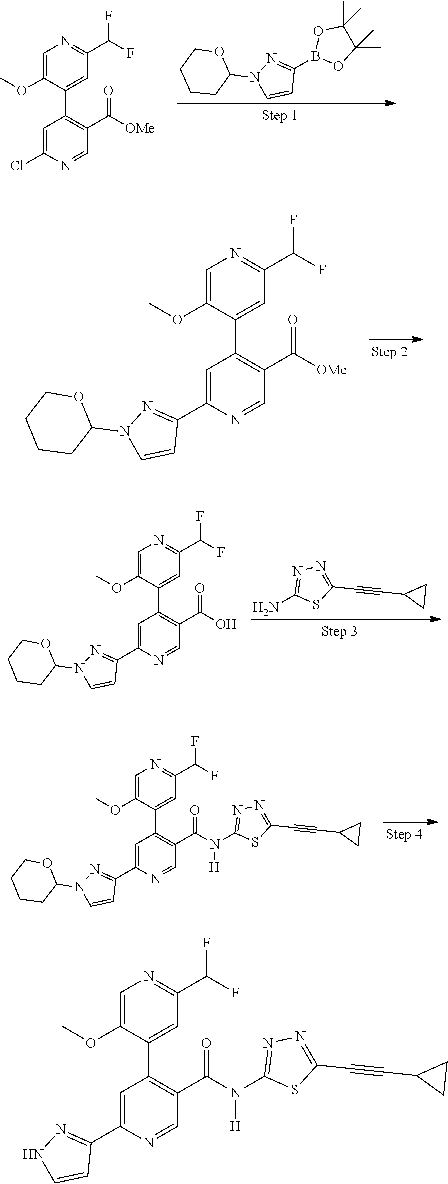

### Step 1/methyl 2-(difluoromethyl)-5′-methoxy-6-(1-(tetrahydro-2H-pyran-2-yl)-1H-pyrazol-3-yl)-[4,4′-bipyridine]-3-carboxylate

A vial was charged with Intermediate 21 (304 mg, 925 μmol) and 1-tetrahydropyran-2-yl-3-(4,4,5,5-tetramethyl-1,3,2-dioxaborolan-2-yl)pyrazole (279 mg, 1.00 mmol), dioxane (3 mL) and K₂CO₃ (2 M, 923 μL, 1.85 mmol). N₂ was bubbled through the mixture, then Pd(OAc)₂ (31.0 mg, 138 μmol) and SPhos (117 mg, 285 μmol) were added. N₂ was bubbled through the mixture again, the vial was capped and heated to 80 °C for 2 h20 min. Additional 1-tetrahydropyran-2-yl-3-(4,4,5,5-tetramethyl-1,3,2-dioxaborolan-2-yl)pyrazole (128.63 mg, 462.43 μmol) was added and the reaction mixture was stirred at 80 °C for 90 min. The final mixture was cooled to rt, filtered on a celite plug, rinsed with EtOAc, diluted with H₂O. The organic layers were separated and the aqueous layer reextracted with EtOAc (2×). The combined organic extracts were washed with brine, dried over Na₂SO₄, filtered, and concentrated in vacuo. The residue was purified by silica gel chromatography eluting with a gradient of EtOAc (20 to 100%) in Hex. Appropriate fractions were combined and concentrated in vacuo to afford methyl 2′-(difluoromethyl)-5′-methoxy-6-(1-(tetrahydro-2H-pyran-2-yl)-1H-pyrazol-3-yl)-[4,4′-bipyridine]-3-carboxylate (219 mg, 53% yield) as an off-white foamy solid. LCMS m/z 445.2 [M+H]⁺.

### Step 2/2′-(difluoromethyl)-5′-methoxy-6-(1-(tetrahydro-2H-pyran-2-yl)-1H-pyrazol-3-yl)-[4,4′-bipyridine]-3-carboxylic acid

To a solution of methyl 2′-(difluoromethyl)-5′-methoxy-6-(1-(tetrahydro-2H-pyran-2-yl)-1H-pyrazol-3-yl)-[4,4′-bipyridine]-3-carboxylate (219 mg, 493 μmol) in MeOH (1.0 mL) and Dioxane (5.0 mL) was added LiOH aqueous (1 M, 990 μL, 0.990 mmol). The resulting mixture was stirred at 60 °C for 45 min. The volatiles were removed in vacuo, the residue was diluted with H₂O and acidified to pH 4-5 with 1N HCl. Solid collected by filtration on Buchner and washed with H₂O, air-dried and dried in vacuo, affording 73 mg beige solid. Filtrate left aside overnight, then acidified again to pH 4, filtered to get a second crop of solid (32 mg). The two crops were merged, affording 2′-(difluoromethyl)-5′-methoxy-6-(1-(tetrahydro-2H-pyran-2-yl)-1H-pyrazol-3-yl)-[4,4′-bipyridine]-3-carboxylic acid (105 mg, 50% yield) as a light beige solid.

LCMS m/z 431.2 [M+H]⁺.

### Step 3/N-(5-(cyclopropylethynyl)-1,3,4-thiadiazol-2-yl)-2′-(difluoromethyl)-5′-methoxy-6-(1-(tetrahydro-2H-pyran-2-yl)-1H-pyrazol-3-yl)-[4,4′-bipyridine]-3-carboxamide

To a vial charged with 2′-(difluoromethyl)-5′-methoxy-6-(1-(tetrahydro-2H-pyran-2-yl)-1H-pyrazol-3-yl)-[4,4′-bipyridine]-3-carboxylic acid (105 mg, 244 μmol), Intermediate 9 (63.0 mg, 381 μmol) and EDC (142 mg, 741 μmol) was added pyridine (2.0 mL). The mixture was stirred overnight at rt then concentrated in vacuo. The residue was adsorbed on silica using DCM then purified by silica gel chromatography eluting with a gradient of MeOH (0 to 20%) in DCM. Appropriate fractions were combined and concentrated in vacuo to afford N-(5-(cyclopropylethynyl)-1,3,4-thiadiazol-2-yl)-2′-(difluoromethyl)-5′-methoxy-6-(1-(tetrahydro-2H-pyran-2-yl)-1H-pyrazol-3-yl)-[4,4′-bipyridine]-3-carboxamide (124 mg, 88% yield) as a beige foamy solid. LCMS m/z 578.1 [M+H]⁺.

### Step 4/Compound 145

To a solution of N-(5-(cyclopropylethynyl)-1,3,4-thiadiazol-2-yl)-2′-(difluoromethyl)-5′-methoxy-6-(1-(tetrahydro-2H-pyran-2-yl)-1H-pyrazol-3-yl)-[4,4′-bipyridine]-3-carboxamide (63 mg, 109 μmol) in MeOH (1.30 mL) was added HCl (4M in Dioxane, 273 μL, 1.09 mmol). The reaction mixture was stirred at rt for 2 h and then concentrated to dryness. The residue was purified by preparative HPLC eluting with a gradient of CH₃CN (20 to 50%) in water both containing 10 mM ammonium bicarbonate (pH adjusted to 10 with NH₄OH). Appropriate fractions were combined and lyophilized to afford Compound 145 (16 mg, 30% yield) as a fluffy white solid. ¹H NMR (400 MHz, DMSO-d₆) δ 13.48 (br s, 1H), 13.29 (br s, 1H), 8.97 (s, 1H), 8.47 (s, 1H), 8.00 (s, 1H), 7.90 (br s, 1H), 7.77 (s, 1H), 7.00 (t, J=55.0 Hz, 1H), 6.98 (s, 1H), 3.67 (s, 3H), 1.70 (tt, J=8.2, 5.0 Hz, 1H), 1.03-0.96 (m, 2H), 0.90-0.83 (m, 2H). ¹⁹F NMR (376 MHz, DMSO-d₆) δ −113.35 (d, J=54.9 Hz). LCMS m/z 494.1 [M+H]⁺.

### Compound 146/Method D/1-(2′-(difluoromethyl)-5′-methoxy-6-((tetrahydro-2H-pyran-3-yl)ethynyl)-[4,4′-bipyridin]-3-yl)-2-(5-((5-methyl-1H-pyrazol-3-yl)ethynyl)-1,3,4-thiadiazol-2-yl)ethan-1-one

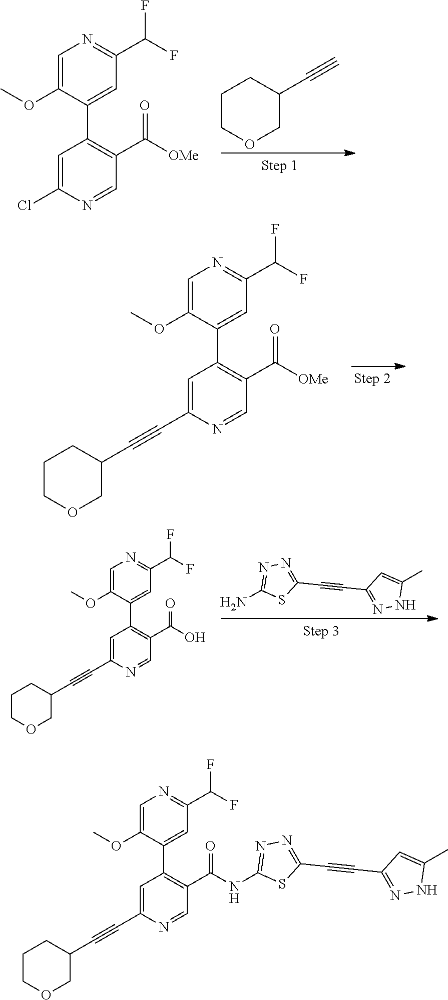

### Step 1/methyl 2′-(difluoromethyl)-5′-methoxy-6-((tetrahydro-2H-pyran-3-yl)ethynyl)-[4,4′-bipyridine]-3-carboxylate

To a vial charged with Intermediate 21 (205 mg, 624 μmol) and 3-ethynyltetrahydropyran (111 mg, 1.01 mmol) was added DMF (3 mL) and DIPEA (197.37 mg, 1.53 mmol, 266 μL). N₂ was bubbled through the solution, then Pd(PPh₃)₄ (70.0 mg, 60.6 μmol) was added. N₂ was bubbled again in the mixture. The vial was capped and the mixture was stirred overnight at 80 °C. The resulting mixture was concentrated to dryness and the residue was purified by silica gel chromatography eluting with a gradient of EtOAc (0 to 100%) in heptane, then MeOH in EtOAc (0-10%). Appropriate fractions were combined and concentrated in vacuo to afford methyl 2′-(difluoromethyl)-5′-methoxy-6-((tetrahydro-2H-pyran-3-yl)ethynyl)-[4,4′-bipyridine]-3-carboxylate (270 mg, 68% pure by HPLC) as a dark orange-brown gum. LCMS m/z 403.2 [M+H]⁺.

### Step 2/2′-(difluoromethyl)-5′-methoxy-6-((tetrahydro-2H-pyran-3-yl)ethynyl)-[4,4′-bipyridine]-3-carboxylic acid

To a vial containing methyl 2′-(difluoromethyl)-5′-methoxy-6-((tetrahydro-2H-pyran-3-yl)ethynyl)-[4,4′-bipyridine]-3-carboxylate (270 mg, 68% pure by HPLC) in Dioxane (3.5 mL) and MeOH (700 μL) was added aqueous LiOH (2 M, 670 μL, 1.34 mmol). The mixture was stirred at 60 °C for 2.5 h, cooled to rt and AcOH (115 μL, 2.01 mmol,) was added. The resulting mixture was concentrated, diluted with H₂O and the pH was adjusted to 4-5 with 1N HCl. The aqueous mixture was extracted with CHCl₃/PrOH (4:1, 4×). The combined organic extracts were concentrated to dryness affording 2′-(difluoromethyl)-5′-methoxy-6-((tetrahydro-2H-pyran-3-yl)ethynyl)-[4,4′-bipyridine]-3-carboxylic acid (270 mg) as a brown gum which was used in the next step without purification. LCMS m/z 389.2 [M+H]⁺.

### Step 3/Compound 146

To a vial charged with 2′-(difluoromethyl)-5′-methoxy-6-((tetrahydro-2H-pyran-3-yl)ethynyl)-[4,4′-bipyridine]-3-carboxylic acid (129 mg, 332 μmol). Intermediate 17 (100 mg, 487 μmol) and EDC (205 mg, 1.07 mmol) was added pyridine (2 mL). The resulting mixture was stirred at rt overnight then concentrated to dryness. The residue was purified by preparative HPLC eluting with a gradient of CH₃CN (25 to 55%) in water containing 10 mM ammonium bicarbonate (pH adjusted to 10 with NH₄OH). Appropriate fractions were combined and lyophilized to afford impure Compound 146. A second purification by preparative HPLC eluting with a gradient of CH₃CN (40 to 70%) in water both containing 0.1% formic acid was performed. Appropriate fractions were combined and lyophilized to afford Compound 146 (16 mg, 8% yield) as a fluffy white solid. ¹H NMR (400 MHz, DMSO-d₆) δ 13.66 (br s, 1H), 13.15 (s, 1H), 8.94 (s, 1H), 8.45 (s, 1H), 7.78 (s, 1H), 7.64 (s, 1H), 6.97 (t, J=55.0 Hz, 1H), 6.42 (s, 1H), 3.89 (ddd, J=11.3, 3.8, 1.3 Hz, 1H), 3.75-3.68 (m, 1H), 3.67 (s, 3H), 3.55-3.39 (m, 2H), 2.88 (tt, J=8.3, 4.0 Hz, 1H), 2.26 (s, 3H), 2.12-1.96 (m, 1H), 1.84-1.62 (m, 2H), 1.55 (s, 1H). ¹⁹F NMR (376 MHz, DMSO-d₆) δ −113.40 (d, J=55.1 Hz). LCMS m/z 576.1 [M+H]⁺.

### Compound 150/Method F/N-(5-(cyclopropylethynyl)-1,3,4-thiadiazol-2-yl)-2′-(difluoromethyl)-5′-methoxy-6-((tetrahydro-2H-pyran-4-yl)methoxy)-[4,4′-bipyridine]-3-carboxamide

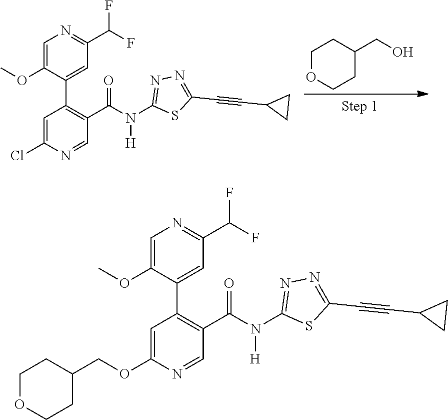

To a solution of tetrahydropyran-4-ylmethanol (125.75 mg, 1.08 mmol) and Intermediate 23 (50.0 mg, 108 μmol) in DMF (1 mL) was added NaH (45.0 mg, 1.13 mmol. 60% purity). The mixture was stirred 5 min at RT and heated at 80 °C for 15 min. After cooling to rt, the mixture was neutralized with AcOH, diluted with DMSO, filtered and the filtrate was purified by preparative HPLC eluting with a gradient of CH₃CN (20 to 80%) in water both containing 0.1% formic acid. Appropriate fractions were combined and lyophilized to afford Compound 150 (34 mg, 58% yield) as a white solid. ¹H NMR (400 MHz, DMSO-d₆) δ 13.26 (s, 1H), 8.56 (s, 1H), 8.38 (s, 1H), 7.66 (s, 1H), 7.09-6.72 (m, 2H), 4.21 (d, J=6.6 Hz, 2H), 3.84 (ddd, J=11.2, 4.5, 1.8 Hz, 2H), 3.60 (s, 3H), 3.26 (s, 2H), 2.00 (m, 1H), 1.70-1.58 (m, 3H), 1.39-1.22 (m, 2H), 0.94 (m, 2H), 0.85-0.78 (m, 2H). LCMS m/z 542.1 [M+H]⁺.

### Compound 268/Method G/N-(5-(cyclopropylethynyl)-1,3,4-thiadiazol-2-yl)-2′-(difluoromethyl)-5′-methoxy-6-((tetrahydro-2H-pyran-4-yl)ethynyl)-[4,4′-bipyridine]-3-carboxamide

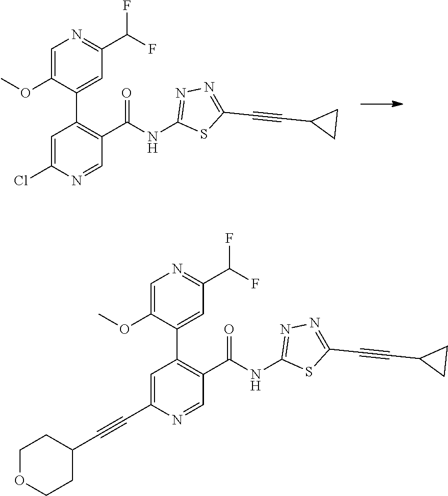 [SMILES: COc1cnc(C(F)F)cc1-c1cc(Cl)ncc1C(=O)Nc1nnc(C#CC2CC2)s1 ; COc1cnc(C(F)F)cc1-c1cc(C#CC2CCOCC2)ncc1C(=O)Nc1nnc(C#CC2CC2)s1]

To a stirred solution of Intermediate 23 (80.0 mg, 0.173 mmol) and 4-ethynyltetrahydro-2H-pyran (38.0 mg, 0.346 mmol) In DMF (0.8 mL) at rt were added Et₃N (72 μL, 0.51 mmol) and CuI (9.0 mg, 0.051 mmol). The reaction mixture was sonicated under N₂ atmosphere for 5 min followed by addition of PdCl₂(dppf)·DCM (10.0 mg, 0.034 mmol). Reaction mixture was heated at 50 °C for 1 h, poured onto crushed ice and extracted with EtOAc (3×10 mL). The combined organic layer was dried over anhydrous Na₂SO₄ and concentrated in vacuo. The residue was purified by silica gel chromatography eluting with a gradient of EtOAc (0 to 50%) in Hexane. It was further purified by preparative HPLC to afford Compound 268 (42.0 mg, 45%). ¹H NMR (400 MHz, DMSO d₆) δ 13.30 (bs, 1H), 9.04 (s, 1H), 8.44 (s, 1H), 7.67 (s, 1H), 7.51 (s, 1H), 6.97 (t, J=55.2 Hz, 1H), 3.86-3.83 (m, 2H), 3.67 (s, 3H), 3.51-3.46 (m, 2H), 3.00 (m, 1H), 1.91-1.88 (m, 2H), 1.68-1.65 (m, 3H), 0.98-0.97 (m, 2H), 0.84 (m, 2H). LCMS m/z 536.0 [M+H]⁺.

### Compound 387/Method D/N-(5-(cyclopropylethynyl)-1,3,4-thiadiazol-2-yl)-2″-(difluoromethyl)-5″-methoxy-4-methyl-2-oxo-2H-[1,2′:4′,4″-terpyridine]-5′-carboxamide

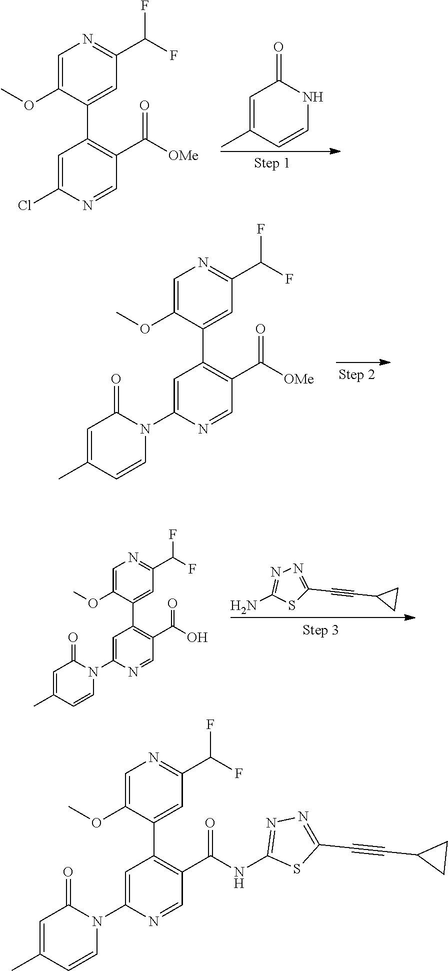

### Step 1/methyl 2″-(difluoromethyl)-5″-methoxy-4-methyl-2-oxo-2H-[1,2′:4′,4″-terpyridine]-5′-carboxylate

To a solution of Intermediate 21 (10.0 g, 61.0 mmol) and 4-methyl-1H-pyridin-2-one (6.65 g, 60.9 mmol) in dry DMSO (100 mL) was added potassium carbonate (8.43 g, 61.0 mmol). The reaction was heated 90 °C (heat block) under N₂ overnight. The reaction mixture was cooled to rt and iodomethane (5.70 mL, 91.6 mmol,) was added and the mixture was stirred for 30 min. The reaction mixture was quenched with saturated NH₄Cl, and the mixture was extracted with EtOAc (2×). The combined organic extracts were washed with H₂O, brine, dried over Na₂SO₄, filtered and concentrated. The residue was purified by silica gel chromatography eluting with a gradient of EtOAc (0 to 100%) in DCM. Appropriate fractions were combined and concentrated in vacuo to afford methyl 2″-(difluoromethyl)-5″-methoxy-4-methyl-2-oxo-2H-[1,2′:4′,4″-terpyridine]-5′-carboxylate (9.01 g, 74% yield) as a white solid. ¹H NMR (400 MHz, DMSO-d₆) δ 8.99 (s, 1H), 8.57 (s, 1H), 7.97 (s, 1H), 7.93 (d, J=7.2 Hz, 1H), 7.67 (s, 1H), 6.99 (t, J=55.0 Hz, 1H), 6.42-6.34 (m, 1H), 6.31 (dd. J=7.3, 1.8 Hz, 1H), 3.89 (s, 3H), 3.73 (s, 3H), 2.20 (d, J=1.1 Hz, 3H). LCMS m/z 402.1 [M+H]⁺.

### Step 2/2″-(difluoromethyl)-5″-methoxy-4-methyl-2-oxo-2H-[1,2′:4′,4″-terpyridine]-5′-carboxylic acid

To a solution of methyl 2″-(difluoromethyl)-5″-methoxy-4-methyl-2-oxo-2H-[1,2′:4′,4″-terpyridine]-5′-carboxylate (9.01 g, 22.5 mmol) in dioxane (200 mL) and MeOH (50 mL) in a water bath was added an aqueous solution of lithium hydroxide (1 M, 45 mL). The bath was removed, and the mixture was stirred at rt for 3 h. 4 M HCl (10 mL) was added to adjust pH to 4-5 and the resulting mixture was concentrated to remove volatiles. H₂O was added, the pH was adjusted to 4 using 4 M HCl, the solid was collected by filtration on Buchner and washed with H₂O. Solids were air-dried overnight, then dried in vacuo, affording 2″-(difluoromethyl)-5″-methoxy-4-methyl-2-oxo-2H-[1,2′:4′,4″-terpyridine]-5′-carboxylic acid (8.16 g, 94% yield) as a white solid. ¹H NMR (400 MHz, DMSO-d₆) δ 13.39 (s, 1H), 8.99 (s, 1H), 8.55 (s, 1H), 7.93 (d, J=7.2 Hz, 1H), 7.90 (s, 1H), 7.63 (s, 1H), 6.98 (t, J=55.0 Hz, 1H), 6.37-6.34 (m, 1H), 6.30 (dd, J=7.3, 1.8 Hz, 1H), 3.89 (s, 3H), 2.20 (d, J=1.2 Hz, 3H). LCMS m/z 388.1 [M+H]⁺.

### Step 3/Compound 387

To a solution of 2″-(difluoromethyl)-5″-methoxy-4-methyl-2-oxo-2H-[1,2′:4′,4″-terpyridine]-5′-carboxylic acid (6.93 g, 17.9 mmol), Intermediate 9 (3.55 g, 21.5 mmol) in pyridine (49 mL) was added EDC (6.87 g, 35.8 mmol) and the resulting mixture was stirred at rt for 3 h. The reaction mixture was concentrated to dryness then the residue was triturated in water, sonicated and the suspension was stirred for 1 h. The solids were collected by filtration on Buchner and washed with H₂O. Air-dried overnight. The hard filter cake was broken down, slurried in acetone and stirred for about 1 h, then the solid was collected by filtration on Buchner and washed with acetone and finally air-dried to afford Compound 387 (8.74 g, 16.35 mmol, 91% yield) as an off-white solid. ¹H NMR (400 MHz, DMSO-d₆) δ 13.59 (s, 1H), 8.98 (s, 1H), 8.48 (s, 1H), 8.03 (s, 1H), 7.93 (d, J=7.2 Hz, 1H), 7.73 (s, 1H), 6.99 (t, J=55.0 Hz, 1H), 6.39-6.34 (m, 1H), 6.32 (dd, J=7.3, 1.9 Hz, 1H), 3.67 (s, 3H), 2.22 (d, J=1.1 Hz, 3H), 1.70 (tt, J=8.2, 5.0 Hz, 1H), 1.06-0.95 (m, 2H), 0.92-0.83 (m, 2H). LCMS m/z 388.1 [M+H]⁺. ¹⁹F NMR (376 MHz, DMSO-d₆) d −113.58 (d, J=55.0 Hz).

### Compound 431/Method D/2″-(difluoromethyl)-N-(5-((1S,2S)-2-ethynylcyclopropyl)-1,3,4-thiadiazol-2-yl)-5″-methoxy-4-methyl-2-oxo-2H-[1,2′:4′,4″-terpyridine]-5′-carboxamide

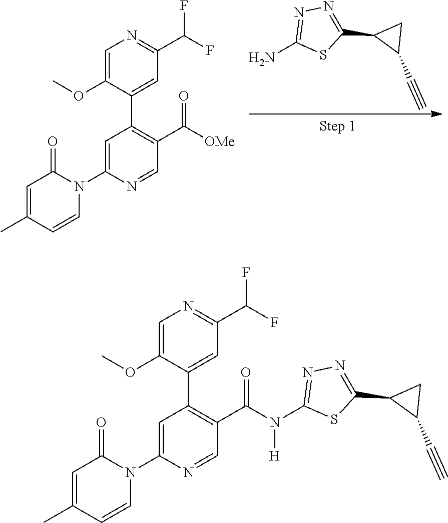 [SMILES: COC(=O)c1cnc(-n2ccc(C)cc2=O)cc1-c1cc(C(F)F)ncc1OC ; C#C[C@H]1C[C@@H]1c1nnc(N)s1 ; C#C[C@H]1C[C@@H]1c1nnc(NC(=O)c2cnc(-n3ccc(C)cc3=O)cc2-c2cc(C(F)F)ncc2OC)s1]

To a mixture of 2″-(difluoromethyl)-5″-methoxy-4-methyl-2-oxo-2H-[1,2′:4′,4″-terpyridine]-5′-carboxylic acid (see Compound 387, Step 2) (1.30 g, 3.36 mmol), Intermediate 26 (665 mg, 4.03 mmol) and EDC (1.29 g, 6.71 mmol) was added pyridine (16 mL). The resulting mixture was stirred at 50 °C for 2 hrs. then cooled to rt and concentrated in vacuo. The residue was triturated in water, sonicated and the resulting mixture was stirred overnight at rt. The resulting solid was filtered, washed with water, and dried. The solid was dissolved in EtOAc and absorbed on silica gel (dry-packed). The mixture was purified by silica gel chromatography eluting with a gradient of MeOH (0 to 10%) in DCM. Appropriate fractions were combined and concentrated in vacuo to afford Compound 431 (1.27 g, 71% yield) as an off-white solid. ¹H NMR (400 MHz, DMSO-d6) δ 13.29 (s, 1H), 8.95 (s, 1H), 8.48 (s, 1H), 8.01 (s, 1H), 7.95-7.91 (m, 1H), 7.73 (s, 1H), 6.99 (t, J=55.0 Hz, 1H), 6.37 (dt, J=1.9, 0.9 Hz, 1H), 6.32 (dd, J=7.3, 1.8 Hz, 1H), 3.68 (s, 3H), 2.94 (d, J=2.1 Hz, 1H), 2.78 (dt, J=9.9, 5.3 Hz, 1H), 2.22 (d, J=1.1 Hz, 3H), 2.00 (dddd, J=8.5, 6.3, 4.4, 2.1 Hz, 1H), 1.57-1.50 (m, 1H), 1.44 (ddd, J=8.7, 6.0, 4.5 Hz, 1H).

LCMS m/z 535.4 [M+H]⁺. Enantiomeric excess of Compound 431 (e.e. >99%) was determined by Supercritical Fluid Chromatography (SFC) using a Lux 5 u Amylose-2/00G-4472-EO (4.6×250 mm) chiral column eluting with CH₃CN-MeOH (1:1) (5 to 60%) with CO₂ (95 to 40%). Under these conditions, Compound 431 has a retention-time of 4.0 minutes, while the enantiomer Compound 480 has a retention-time of 4.2 minutes.

### Compound 432/Method D/N-(5-(cyclopropylethynyl)-1,3,4-thiadiazol-2-yl)-2′-(difluoromethyl)-5′-methoxy-6-(4-methyl-8-oxo-4,7-diazaspiro[2.5]octan-7-yl)-[4,4′-bipyridine]-3-carboxamide

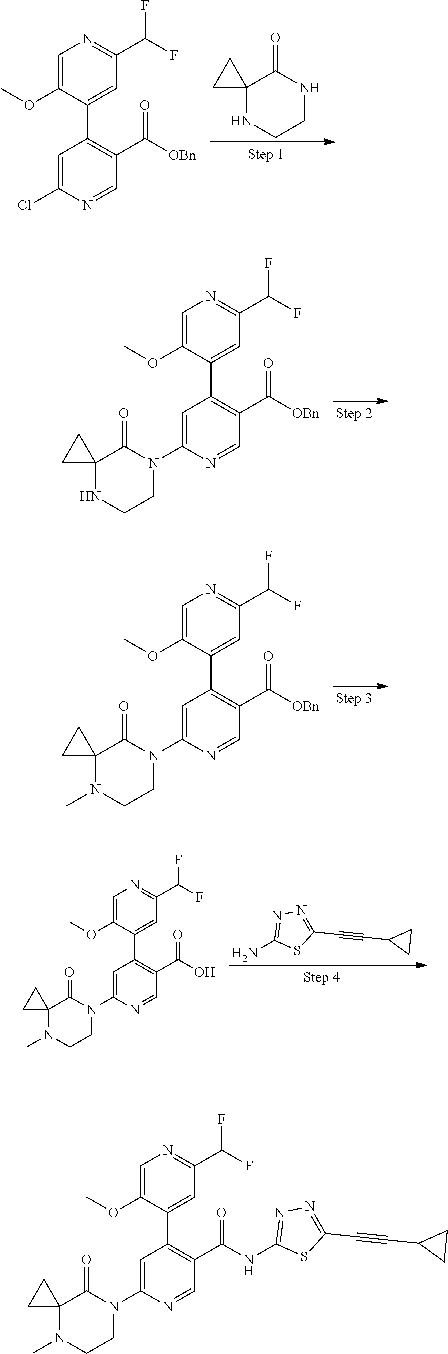

### Step 1/benzyl 2′-(difluoromethyl)-5′-methoxy-6-(8-oxo-4,7-diazaspiro[2.5]octan-7-yl)-[4,4′-bipyridine]-3-carboxylate

A suspension of Intermediate 25 (4.99 g, 12.3 mmol), 4,7-diazaspiro[2.5]octan-8-one (1.87 g, 14.8 mmol) and cesium carbonate (8.02 mg, 24.6 μmol) in dry dioxane (50 mL) was degassed (N₂ bubbling/sonication for 15 min). Pd(OAc)₂ (278 mg, 1.23 mmol) and Xantphos (1.07 g, 1.85 mmol) were added and the mixture degassed again (5 min). The reaction mixture was stirred in a heat block at 80 °C for 1.5 h. The reaction mixture was cooled to rt, filtered on celite, and the solids were washed with DCM. The combined filtrates were concentrated, and the residue was purified by silica gel chromatography eluting with a gradient of 1:1 EtOAc/IPA (0 to 100%) in heptane. Appropriate fractions were combined and concentrated in vacuo to afford benzyl 2′-(difluoromethyl)-5′-methoxy-6-(8-oxo-4,7-diazaspiro[2.5]octan-7-yl)-[4,4′-bipyridine]-3-carboxylate (5.23 g, 86% yield) as a beige foamy solid. ¹H NMR (400 MHz, DMSO-d₆) δ 9.03-8.89 (m, 1H), 8.43 (s, 1H), 8.04 (s, 1H), 7.59 (s, 1H), 7.39-7.29 (m, 3H), 7.23-6.76 (m, 3H), 5.18 (s, 2H), 4.13 (t, J=5.5 Hz, 2H), 3.76 (s, 3H), 3.28 (t, J=7.2 Hz, 1H), 3.15 (q, J=6.0 Hz, 2H), 1.28 (q, J=3.5 Hz, 2H), 0.93-0.85 (m, 2H). LCMS m/z 495.1 [M+H]⁺.

### Step 2/benzyl 2′-(difluoromethyl)-5′-methoxy-6-(4-methyl-8-oxo-4,7-diazaspiro[2.5]octan-7-yl)-[4,4′-bipyridine]-3-carboxylate

To a solution of benzyl 2′-(difluoromethyl)-5′-methoxy-6-(8-oxo-4,7-diazaspiro[2.5]octan-7-yl)-[4,4′-bipyridine]-3-carboxylate (4.19 g, 8.47 mmol) in THF (41 mL) at rt was added formaldehyde (14.17 g, 165.2 mmol, 13 mL, 35% purity) and sodium triacetoxyborohydride (5.39 g, 25.2 mmol) The reaction mixture was stirred at 40 °C for 20 min. The crude reaction mixture was cooled to rt and NaHCO₃ sat. (100 mL) was added dropwise under stirring. EtOAc was added, the layers were separated, and the aqueous layer was back extracted with EtOAc. The combined organic extracts were washed with brine, dried over Na₂SO₄, filtered on a silica plug, washed with EtOAc and concentrated in vacuo. The residue was purified by silica gel chromatography eluting with a gradient of 1:1 EtOAc/IPA (0 to 100%) in hexane. Appropriate fractions were combined and concentrated in vacuo to afford benzyl 2′-(difluoromethyl)-5′-methoxy-6-(4-methyl-8-oxo-4,7-diazaspiro[2.5]octan-7-yl)-[4,4′-bipyridine]-3-carboxylate (3.71 g, 86% yield) as a light beige foamy solid. ¹H NMR (400 MHz, DMSO-d₆) b 8.94 (s, 1H), 8.38 (s, 1H), 8.02 (s, 1H), 7.56 (s, 1H), 7.40-7.23 (m, 3H), 7.22-6.73 (m, 3H), 5.14 (s, 2H), 4.22 (dd, J=6.5, 5.3 Hz, 2H), 3.72 (s, 3H), 3.26 (t, J=5.9 Hz, 2H), 2.45 (s, 3H), 1.25 (q, J=3.8 Hz, 2H), 0.97 (q, J=3.7 Hz, 2H). LCMS m/z 509.2 [M+H]⁺.

### Step 3/2′-(difluoromethyl)-5′-methoxy-6-(4-methyl-8-oxo-4,7-diazaspiro[2.5]octan-7-yl)-[4,4′-bipyridine]-3-carboxylic acid

To a solution benzyl 2′-(difluoromethyl)-5′-methoxy-6-(4-methyl-8-oxo-4,7-diazaspiro[2.5]octan-7-yl)-[4,4′-bipyridine]-3-carboxylate (3.71 g, 7.30 mmol) in MeOH (100 mL) and DCM (50 mL). The flask was flushed with N₂. Palladium on activated carbon (751 mg, 706 μmol, 10% purity) was added. The flask was flushed with H₂ and stirred under a H₂ atmosphere overnight. The reaction mixture was purged with N₂, diluted with 100 mL DCM then filtered on a celite plug prewashed with MeOH, catalyst carefully washed with portions of MeOH, and DCM. The filtrate was concentrated and dried in vacuo, providing 2′-(difluoromethyl)-5′-methoxy-6-(4-methyl-8-oxo-4,7-diazaspiro[2.5]octan-7-yl)-[4,4′-bipyridine]-3-carboxylic acid (2.78 g, 91% yield) as a light yellow solid. ¹H NMR (400 MHz, DMSO-d₆) δ 13.07 (s, 1H), 8.90 (d, J=0.6 Hz, 1H), 8.51 (s, 1H), 7.98 (d, J=0.6 Hz, 1H), 7.53 (s, 1H), 6.97 (t, J=55.1 Hz, 1H), 4.22 (dd, J=6.6, 5.3 Hz, 2H), 3.86 (s, 3H), 3.26 (d, J=11.9 Hz, 1H), 2.46 (s, 3H), 1.25 (q, J=3.8 Hz, 2H), 0.97 (q, J=3.8 Hz, 2H). LCMS m/z 419.1 [M+H]⁺.

### Step 4/Compound 432

To a solution of 2′-(difluoromethyl)-5′-methoxy-6-(4-methyl-8-oxo-4,7-diazaspiro[2.5]octan-7-yl)-[4,4′-bipyridine]-3-carboxylic acid (2.78 g, 6.64 mmol), Intermediate 9 (1.32 g, 7.98 mmol) in pyridine (40 mL) was added EDC (2.55 g, 13.3 mmol) and the resulting mixture was stirred at rt overnight. The reaction mixture was concentrated to dryness then the residue was triturated in water, sonicated and the suspension was stirred for 1 h. The solids were collected by filtration on Buchner, washed with H₂O and air-dried. The cake was triturated with IPA and collected by filtration, washed with small amounts of IPA and then dried in vacuo to afford Compound 432 (3.25 g, 86% yield) as an off-white solid. ¹H NMR (400 MHz, DMSO-d₆) δ 13.46 (s, 1H), 8.85 (s, 1H), 8.45 (s, 1H), 8.07 (s, 1H), 7.64 (s, 1H), 7.00 (t, J=55.1 Hz, 1H), 4.23 (t, J=5.9 Hz, 2H), 3.65 (s, 3H), 3.28 (t, J=5.9 Hz, 2H), 2.48 (s, 3H), 1.70 (tt, J=8.3, 5.0 Hz, 1H), 1.27 (q, J=3.8 Hz, 2H), 1.04-0.95 (m, 4H), 0.87 (dt, J=4.9, 3.2 Hz, 2H). LCMS m/z 566.2 [M+H]⁺. ¹⁹F NMR (376 MHz, DMSO-d₆) δ −113.46 (d, J=55.3 Hz).

### Compound 562/Method A/2″-(difluoromethyl)-N-(5-((1S,2S)-2-ethynylcyclopropyl)-1,3,4-thiadiazol-2-yl)-3-fluoro-5″-methoxy-2-oxo-2H-[1,2′:4′,4″-terpyridine]-5′-carboxamide

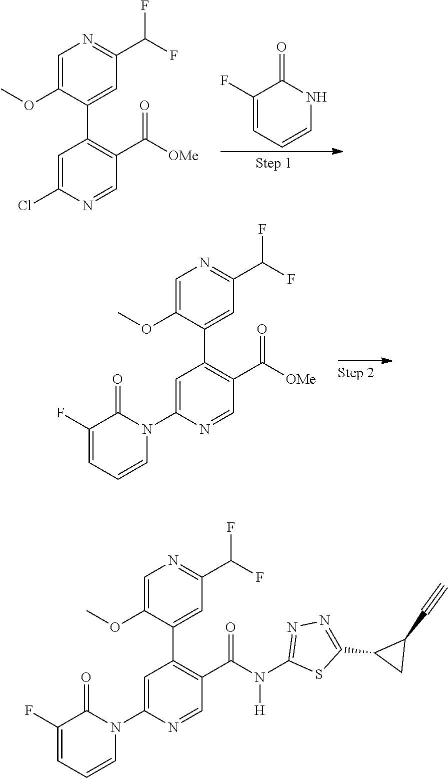 [SMILES: COC(=O)c1cnc(Cl)cc1-c1cc(C(F)F)ncc1OC ; O=c1[nH]cccc1F ; COC(=O)c1cnc(-n2cccc(F)c2=O)cc1-c1cc(C(F)F)ncc1OC ; C#C[C@H]1C[C@@H]1c1nnc(NC(=O)c2cnc(-n3cccc(F)c3=O)cc2-c2cc(C(F)F)ncc2OC)s1]

### Step 1/methyl 2″-(difluoromethyl)-3-fluoro-5″-methoxy-2-oxo-2H-[1,2′:4′,4″-terpyridine]-5′-carboxylate

A mixture of Intermediate 21 (300 mg, 913 μmol), 3-fluoro-1H-pyridin-2-one (310 mg, 2.74 mmol), K₂CO₃ (375 mg, 2.71 mmol) In DMSO (3.0 mL) was stirred overnight in a sealed vial at 50 °C. The reaction mixture was cooled to rt, diluted with EtOAc and water. The aqueous layer was cut, and the organic layer was washed with water, brine (2×), dried over Na₂SO₄, filtered and concentrated in vacuo. The residue was purified by silica gel chromatography eluting with a gradient of EtOAc (40 to 100%) in heptane. Appropriate fractions were combined and concentrated in vacuo to afford methyl 2″-(difluoromethyl)-3-fluoro-5″-methoxy-2-oxo-2H-[1,2′:4′,4″-terpyridine]-5′-carboxylate (256 mg, 69% yield) as a white solid. LCMS m/z 406.1 [M+H]⁺.

### Step 2/Compound 562

To a solution of methyl 2″-(difluoromethyl)-3-fluoro-5″-methoxy-2-oxo-2H-[1,2′:4′,4″-terpyridine]-5′-carboxylate (80.0 mg, 197 μmol) and Intermediate 26 (98.0 mg, 593 μmol) in THF (2 mL) was added 1,3,4,6,7,8-hexahydro-2H-pyrimido[1,2-a]pyrimidine (42.0 mg, 302 μmol) in one shot. The reaction was stirred at 50 °C overnight. The cooled crude reaction mixture was diluted with DMSO, filtered, and the filtrate was purified by preparative HPLC (Phenomenex Gemini®) eluting with a gradient of CH₃CN (35 to 65%) in water both containing 0.1% formic acid. Appropriate fractions were combined and lyophilized to afford Compound 562 (35 mg, 33% yield). ¹H NMR (DMSO-d₆) δ: 13.33 (s, 1H), 8.99 (s, 1H), 8.50 (s, 1H), 8.06 (s, 1H), 7.86 (dt, J=7.1, 1.5 Hz, 1H), 7.73 (s, 1H), 7.56 (ddd, J=10.3, 7.4, 1.8 Hz, 1H), 7.00 (t, J=55.0 Hz, 1H), 6.42 (td, J=7.3, 4.7 Hz, 1H), 3.69 (s, 3H), 2.94 (d, J=2.1 Hz, 1H), 2.84-2.74 (m, 1H), 2.04-1.96 (m, 1H), 1.59-1.49 (m, 1H), 1.49-1.40 (m, 1H). LCMS m/z 539.3 [M+H]⁺.

### Compound 583/Method A/2″-(difluoromethyl)-5″-methoxy-4-methyl-N-(5-((1S,2S)-2-(1-methyl-1H-pyrazol-3-yl)cyclopropyl)-1,3,4-thiadiazol-2-yl)-2-oxo-2H-[1.2′:4′,4″-terpyridine]-5′-carboxamide

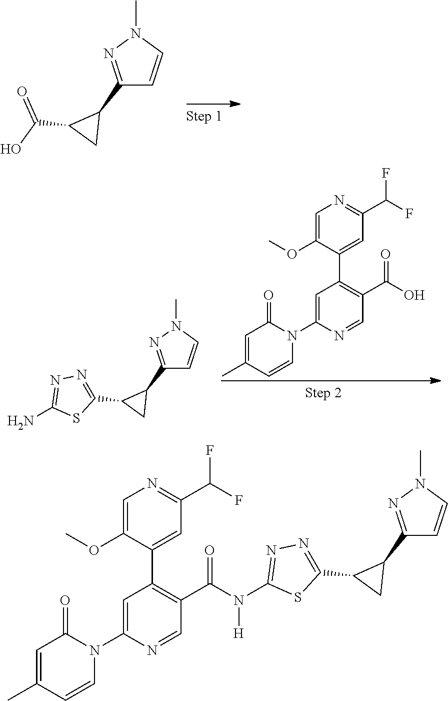

### Step 1/5-((1S,2S)-2-(1-methyl-1H-pyrazol-3-yl)cyclopropyl)-1,3,4-thiadiazol-2-amine

To the solution of (1S,2S)-2-(1-methyl-1H-pyrazol-3-yl)cyclopropane-1-carboxylic acid (100 mg, 604 μmol) were added T3P 50% wt in EtOAc (898 uL, 1.51 mmol, 50% purity), Thiosemicarbazide (66.0 mg, 724 μmol) at rt. The reaction mixture was stirred at 95 °C for 16 hrs in a sealed tube. The resulting mixture was cooled to rt, diluted with saturated NaHCO₃, and extracted with EtOAc (3×). The combined organic layers were washed with brine, dried over MgSO₄, filtered and concentrated under vacuum to get crude product 5-((1S,2S)-2-(1-methyl-1H-pyrazol-3-yl)cyclopropyl)-1,3,4-thiadiazol-2-amine (12.0 mg, 8% yield, 87% purity). LCMS m/z 222.0 [M+H]⁺.

### Step 2/Compound 583

A mixture of 2″-(difluoromethyl)-5″-methoxy-4-methyl-2-oxo-2H-[1,2′:4′,4″-terpyridine]-5′-carboxylic acid (see Compound 387, Step 2) (15.0 mg, 38.7 μmol), 5-((1S,2S)-2-(1-methyl-1H-pyrazol-3-yl)cyclopropyl)-1,3,4-thiadiazol-2-amine (12.0 mg, 53.1 μmol) and EDC (14.9 mg, 77.5 μmol) In pyridine (0.3 mL) was stirred at 50 °C for 30 mins. The crude reaction mixture was concentrated, dissolved in DMSO, filtered, and the filtrate was purified by preparative HPLC (Phenomenex Gemini®) eluting with a gradient of CH₃CN (25 to 55%) in water both containing 0.1% formic acid. Appropriate fractions were combined and lyophilized to afford Compound 583 (7.9 mg, 35% yield) as an off-white solid. ¹H NMR (400 MHz, DMSO-d₆) δ 13.25 (s, 1H), 8.96 (s, 1H), 8.48 (s, 1H), 7.99 (s, 1H), 7.93 (d, J=7.3 Hz, 1H), 7.72 (s, 1H), 7.56 (d, J=2.2 Hz, 1H), 6.99 (t, J=55.1 Hz, 1H), 6.37 (dt, J=2.1, 1.1 Hz, 1H), 6.32 (dd, J=7.3, 1.8 Hz, 1H), 6.10 (d, J=2.2 Hz, 1H), 3.75 (s, 3H), 3.69 (s, 3H), 2.63 (dt, J=9.3, 5.0 Hz, 1H), 2.48 (s, 1H), 2.22 (d, J=1.2 Hz, 3H), 1.65-1.51 (m, 2H). LCMS m/z 591.3 [M+H]⁺.

### Compound 634/Method H/2′-(difluoromethyl)-5′-methoxy-6-(3-morpholinoprop-1-yn-1-yl)-N-(5-((1S,2S)-2-(thiazol-4-yl)cyclopropyl)-1,3,4-thiadiazol-2-yl)-[4,4′-bipyridine]-3-carboxamide

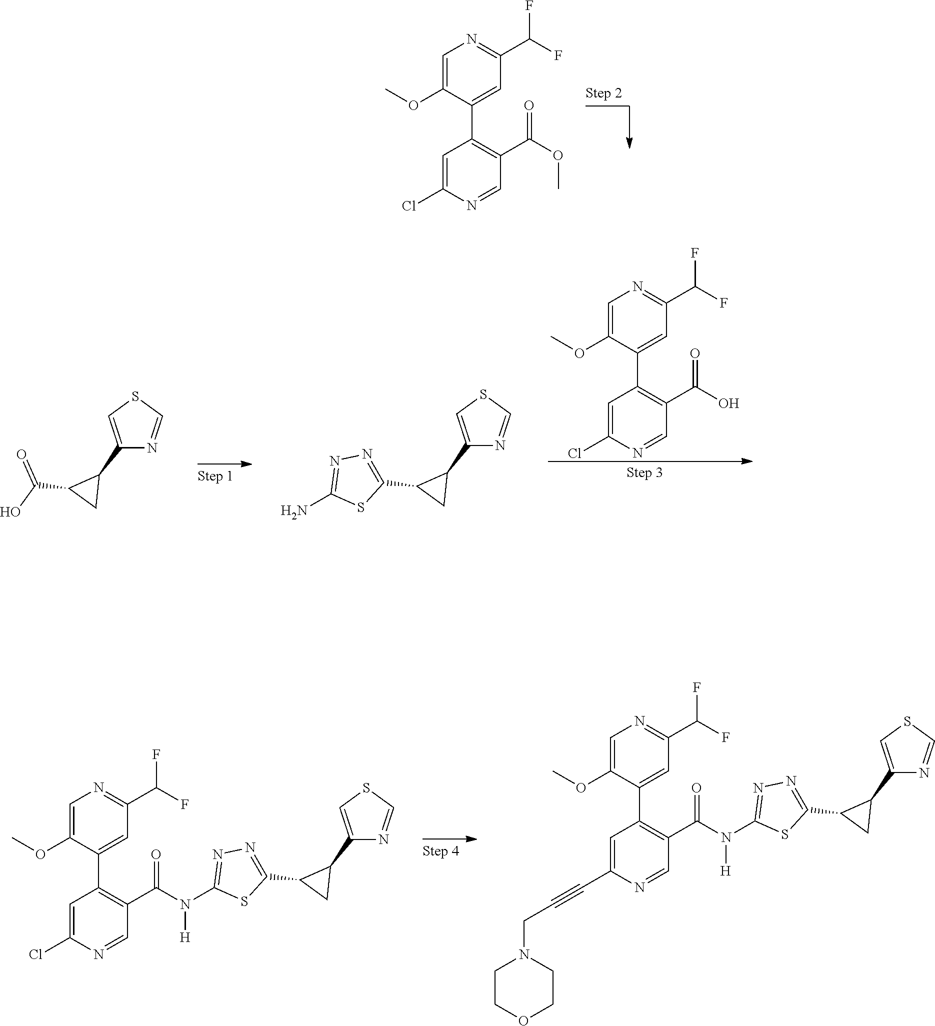

### Step 1/5-((1S,2S)-2-(thiazol-4-yl)cyclopropyl)-1,3,4-thiadiazol-2-amine

To the solution of (1R,2R)-2-thiazol-4-ylcyclopropanecarboxylic acid (trans, racemic) (1.00 g, 5.91 mmol) were added T3P in EtOAc (8.80 mL, 14.8 mmol, 50% purity) and thiosemicarbazide (646 mg, 7.09 mmol) at rt. The reaction mixture was stirred at 95 °C for 16 hrs in a sealed tube. The reaction mixture was cooled to rt, diluted with saturated NaHCO₃ and extracted with EtOAc (3×). The combined organic layers were washed with brine, dried over anhydrous Na₂SO₄, filtered, and concentrated under vacuum to get crude 5-((1S,2S)-2-(thiazol-4-yl)cyclopropyl)-1,3,4-thiadiazol-2-amine (0.95 g, 72% yield) which was used in the next step without purification. LCMS m/z 225.0 [M+H]⁺.

### Step 2/6-chloro-4-[2-(difluoromethyl)-5-methoxy-4-pyridyl]pyridine-3-carboxylic acid

Intermediate 21 (2.00 g, 5.84 mmol) was dissolved in methanol (8 mL) and dioxane (20 mL) before the addition of LiOH·H₂O (490 mg, 11.7 mmol) in water (6 mL) and heated to 60 °C for 15 min. Volatiles were removed in vacuo, 10 mL water was added followed by dropwise addition of HCl (2 M, 5.9 mL, 11.8 mmol). The precipitate was filtered, dried on mechanical pump to afford 6-chloro-4-[2-(difluoromethyl)-5-methoxy-4-pyridyl]pyridine-3-carboxylic acid (1.84 g, 100% yield) as a beige solid. LCMS m/z 315.0 [M+H]⁺.

### Step 3/6-chloro-2′-(difluoromethyl)-5′-methoxy-N-(5-((1S,2S)-2-(thiazol-4-yl)cyclopropyl)-1,3,4-thiadiazol-2-yl)-[4,4′-bipyridine]-3-carboxamide

A mixture of 6-chloro-4-[2-(difluoromethyl)-5-methoxy-4-pyridyl]pyridine-3-carboxylic acid (135 mg, 429 μmol), 5-[(1S,2S)-2-thiazol-4-ylcyclopropyl]-1,3,4-thiadiazol-2-amine (97.0 mg, 433 μmol), EDC (130 mg, 678 μmol) in pyridine (2.0 mL) was heated at 50 °C for 15 min. The reaction mixture was cooled to rt poured into water (100 mL) and the resulting solid was filtered to afford 6-chloro-2′-(difluoromethyl)-5′-methoxy-N-(5-((1S,2S)-2-(thiazol-4-yl)cyclopropyl)-1,3,4-thiadiazol-2-yl)-[4,4′-bipyridine]-3-carboxamide (203 mg, 91% yield) which was used in the next step without purification. LCMS m/z 521.2 [M+H]⁺.

### Step 4/Compound 634

A mixture of 6-chloro-2′-(difluoromethyl)-5′-methoxy-N-(5-((1S,2S)-2-(thiazol-4-yl)cyclopropyl)-1,3,4-thiadiazol-2-yl)-[4,4′-bipyridine]-3-carboxamide (70.0 mg, 134 μmol). 4-prop-2-ynylmorpholine (16.8 mg, 134 μmol), TEA (75 μL, 534 μmol), CuI (2.6 mg, 13 μmol), Pd(dppf)Cl₂·DCM (11.0 mg, 13.5 μmol) in DMF was heated at 90 °C for 20 min. The crude reaction mixture was purified directly by preparative HPLC (Phenomenex Gemini®) eluting with a gradient of CH₃CN (30 to 60%) in water both containing 0.1% formic acid. The relevant fractions were combined and lyophilized to afford Compound 634 (11.0 mg, 13% yield). ¹H NMR (DMSO-d₆) δ:13.22 (s, 1H), 9.03 (d, J=2.0 Hz, 1H), 8.92 (s, 1H), 8.46 (s, 1H), 7.75 (s, 1H), 7.68 (s, 1H), 7.53 (d, J=2.0 Hz, 1H), 6.97 (t, J=55.0 Hz, 1H), 3.68 (s, 3H), 3.65-3.56 (m, 6H), 2.81 (t. J=7.3 Hz, 2H), 2.55 (t, J=4.7 Hz, 4H), 1.78-1.64 (m, 2H). LCMS m/z 610.0 [M+H]⁺.

### Spray Dried Dispersion (SDD) Formulation

A spray dried dispersion (SDD) formulation was developed using Compound 113. A 1:1 (w/w) mixture of Compound 113 and HPMCAS (grade H) were fully dissolved in a 4:1 (v/v) mixture of dichloromethane:methanol at a loading of ~5% solid (w/w). The solution was spray-dried to an amorphous powder which was further dried overnight in a vacuum oven set to 40 °C to remove residual solvents. This SDD material was used for subsequent in vivo dosing in mouse models.

### Polθ ATPase Enzymatic Assay (3 nM Enzyme Concentration)

PolQ ATPase enzyme (1-894) at 3 nM is incubated with a 10-point concentration response of inhibitors for 15 min at rt in the following buffer: 50 mM Tris Cl pH 7.5, 10% glycerol, 5 mM DTT, 10 mM MgCl₂, 0.1 mg/ml BSA. Following pre-incubation with inhibitors, DNA (Fork C) at 20 nM and ATP at 100 μM are added to start the reaction. Enzymatic reaction proceeds at rt for 60 min. ATP consumption is measured using the ADP-Glo assay from Promega. Luminescence is read on the Envision and IC50 are determined using the variable slope 4-parameters equation.

DNA (Fork C) is made by annealing 3 different oligos containing the following sequence of nucleotides:

Fork 44:
(SEQ ID NO: 1)
5′-GCACTGGCCGTCGTTTTACGGTCGTGACTGGGAAAACCCTGGCG-3′
Fork 45:
(SEQ ID NO: 2)
5′-TTTTTTTTTTTTTTTTTTTTTTCCAAGTAAAACGACGGCCAGTGC-
3′
Fork 26:
(SEQ ID NO: 3)
5′-TTGGAAAAAAAAAAAAAAAAAAAAAA-3′

Oligos are annealed by heating at 95 °C for 5 min in the following buffer (10 mM Tris-HCl pH7.5, 50 mM NaCl, 1 mM EDTA) and cooled to rt.

### Polθ ATPase Enzymatic Assay (0.5 nM Enzyme Concentration)

Polθ ATPase enzyme (1-894) at 0.5 nM is incubated with a 10-point concentration response of inhibitors for 15 min at rt in the following buffer: 50 mM Tris Cl pH 7.5, 10% glycerol. 5 mM DTT, 10 mM MgCl₂, 0.1 mg/ml BSA. Following pre-incubation with inhibitors, DNA (Fork C) at 20 nM and ATP at 100 μM are added to start the reaction. Enzymatic reaction proceeds at rt for 180 min. ATP consumption is measured using the ADP-Glo assay from Promega. Luminescence is read on the Envision and IC₅₀ are determined using the variable slope 4-parameters equation.

Oligos are annealed by heating at 95 °C for 5 min in the following buffer (10 mM Tris-HCl pH7.5, 50 mM NaCl. 1 mM EDTA) and cooled to rt.

In Table 4, the Compounds were prepared according to Methods described previously using Intermediates described herein, commercially available reagents or intermediates described in the literature. In some cases, the use of protecting groups may be required to prepare Compounds described below. The m/z [M+H]⁺ column indicates the positive ion mass observed by LCMS (ESI). The IC₅₀ (nM) column relates to average IC₅₀'s generated in the PolQ ATPase enzymatic assays using either 3 nM or 0.5 nM of PolQ ATPase enzyme (1-894).

TABLE 4

| | | m/z | IC₅₀ (nM) | IC₅₀ (nM) |
|---|---|---|---|---|
| Compound | Method | [M + H]⁺ | [PolQ] 0.5 nM | [PolQ] 3 nM |
| 1 | A | 471.9 | 1.1 | 5.8 |
| 2 | A | 479.1 | 2.2 | 3.3 |
| 3 | A | 451.0 | | 23 |
| 4 | A | 501.4 | | 92 |
| 5 | B | 416.1 | 1.5 | 2.9 |
| 6 | B | 428.0 | 0.63 | 2.0 |
| 7 | C | 418.1 | | 12 |
| 8 | B | 460.2 | | 48 |
| 9 | B | 417.1 | | 33 |
| 10 | C | 487.2 | | 54 |
| 11 | C | 472.2 | | 117 |
| 12 | D | 402.2 | 3.5 | 2.0 |
| 13 | D | 395.2 | 4.2 | 6.9 |
| 14 | C | 472.1 | | 139 |
| 15 | A | 509.1 | | 15 |
| 16 | A | 557.1 | | 7.5 |
| 17 | C | 465.0 | 3.2 | 5.4 |
| 18 | C | 534.3 | | 204 |
| 19 | C | 465.0 | | 27 |
| 20 | D | 432.2 | | 21 |
| 21 | D | 425.2 | | 12 |
| 22 | D | 384.2 | | 12 |
| 23 | D | 390.9 | | 134 |
| 24 | D | 445.1 | 0.78 | <1.5 |
| 25 | D | 412.1 | 1.3 | 1.5 |
| 26 | A | 489.1 | 0.53 | 2.6 |
| 27 | D | 485.1 | 0.26 | |
| 28 | D | 452.1 | 0.36 | |
| 29 | D | 438.9 | 0.60 | |
| 30 | A | 522.2 | 0.76 | |
| 31 | D | 428.2 | 0.63 | |
| 32 | D | 442.1 | 0.66 | |
| 33 | D | 530.2 | <0.25 | |
| 34 | D | 550.2 | 0.29 | |
| 35 | D | 496.6 | <0.25 | |
| 36 | D | 468.2 | 6.6 | |
| 37 | D | 458.0 | 3.3 | |
| 38 | E | 411.8 | 1.1 | |
| 39 | D | 494.2 | 0.27 | |
| 40 | D | 436.3 | <0.25 | |
| 41 | D | 479.0 | <0.25 | |
| 42 | D | 483.1 | 1.8 | |
| 43 | D | 395.0 | <0.25 | |
| 44 | D | 416.0 | 0.31 | |
| 45 | D | 489.2 | 2.2 | |
| 46 | D | 477.3 | 0.20 | |
| 47 | D | 514.0 | 0.62 | |
| 48 | D | 493.7 | 2.5 | |
| 49 | D | 484.1 | 0.75 | |
| 50 | D | 488.1 | 2.4 | |
| 51 | D | 547.2 | 2.1 | |
| 52 | D | 533.0 | 2.4 | |
| 53 | D | 535.2 | 0.31 | |
| 54 | D | 507.2 | <0.25 | |
| 55 | D | 453.2 | <0.25 | |
| 56 | D | 427.2 | <0.25 | |
| 57 | D | 411.7 | 0.37 | |
| 58 | D | 502.6 | 0.91 | |
| 59 | D | 476.7 | 1.89 | |
| 60 | D | 501.1 | 1.10 | |
| 61 | D | 561.2 | <0.25 | |
| 62 | A | 530.2 | 0.36 | |
| 63 | D | 524.7 | 1.9 | |
| 64 | D | 478.1 | 1.9 | |
| 65 | D | 562.0 | 2.2 | |
| 66 | D | 500.2 | 0.60 | |
| 67 | D | 536.2 | <0.25 | |
| 68 | D | 413.1 | 1.6 | |
| 69 | D | 472.1 | 0.28 | |
| 70 | D | 392.3 | 2.5 | |
| 71 | D | 410.2 | 1.8 | |
| 72 | D | 437.1 | 0.62 | |
| 73 | D | 455.1 | 0.42 | |
| 74 | D | 478.0 | 0.73 | |
| 75 | D | 546.3 | <0.25 | |
| 76 | D | 521.2 | <0.25 | |
| 77 | D | 535.2 | <0.25 | |
| 78 | D | 557.2 | 2.71 | |
| 79 | D | 418.4 | 1.21 | |
| 80 | D | 475.1 | 2.1 | |
| 81 | D | 586.1 | <0.25 | |
| 82 | D | 572.3 | <0.25 | |
| 83 | D | 511.1 | <0.25 | |
| 84 | D | 525.2 | 0.26 | |
| 85 | D | 432.2 | 1.10 | |
| 86 | D | 525.2 | 1.6 | |
| 87 | D | 500.2 | 0.36 | |
| 88 | D | 486.1 | 2.4 | |
| 89 | D | 533.1 | 1.2 | |
| 90 | A | 523.1 | 0.8 | |
| 91 | D | 446.1 | 2.3 | |
| 92 | A | 609.2 | 0.25 | |
| 93 | D | 532.2 | 0.32 | |
| 94 | D | 558.2 | <0.25 | |
| 95 | D | 518.2 | <0.25 | |
| 96 | D | 452.1 | 2.6 | |
| 97 | D | 492.2 | 2.0 | |
| 98 | D | 518.2 | 0.54 | |
| 99 | D | 513.2 | 0.36 | |
| 100 | D | 438.1 | <0.25 | |
| 101 | D | 446.2 | <0.25 | |
| 102 | D | 515.2 | 1.2 | |
| 103 | D | 482.1 | 1.2 | |
| 104 | D | 499.2 | 0.31 | |
| 105 | D | 485.2 | 0.47 | |
| 106 | D | 488.1 | <0.25 | |
| 107 | D | 468.2 | 0.76 | |
| 108 | D | 526.2 | <0.25 | |
| 109 | D | 539.4 | <0.25 | |
| 110 | D | 527.3 | <0.25 | |
| 111 | D | 509.2 | 0.78 | |
| 112 | D | 466.2 | <0.25 | |
| 113 | D | 508.1 | 0.53 | |
| 114 | E | 542.0 | 0.26 | |
| 115 | E | 560.0 | <0.25 | |
| 116 | E | 522.1 | <0.25 | |
| 117 | E | 507.2 | 2.0 | |
| 118 | D | 538.4 | 0.99 | |
| 119 | E | 537.4 | 23 | |
| 120 | E | 508.1 | 2.5 | |
| 121 | D | 540.9 | 2.0 | |
| 122 | D | 520.8 | <0.25 | |
| 123 | D | 582.8 | <0.25 | |
| 124 | D | 584.7 | <0.25 | |
| 125 | F | 577.1 | 21 | |
| 126 | E | 523.1 | <0.25 | |
| 127 | D | 508.1 | <0.25 | |
| 128 | F | 540.1 | 4.0 | |
| 129 | F | 594.2 | 1.6 | |
| 130 | F | 553.1 | 2.1 | |
| 131 | E | 505.7 | <0.25 | |
| 132 | E | 539.7 | <0.25 | |
| 133 | D | 562.7 | <0.25 | |
| 134 | D | 536.8 | <0.25 | |
| 135 | D | 506.1 | <0.25 | |
| 136 | D | 550.1 | 8.5 | |
| 137 | D | 566.8 | 0.26 | |
| 138 | D | 525.3 | <0.25 | |
| 139 | D | 507.0 | 61 | |
| 140 | D | 509.3 | 38 | |
| 141 | D | 539.1 | <0.25 | |
| 142 | D | 537.0 | <0.25 | |
| 143 | D | 524.9 | <0.25 | |
| 144 | D | 624.9 | 0.26 | |
| 145 | D | 494.1 | <0.25 | |
| 146 | D | 576.1 | <0.25 | |
| 147 | F | 555.1 | 0.31 | |
| 148 | F | 528.1 | 0.93 | |
| 149 | F | 528.1 | 0.37 | |
| 150 | F | 542.1 | <0.25 | |
| 151 | D | 536.1 | <0.25 | |
| 152 | D | 523.8 | 1.2 | |
| 153 | D | 549.0 | <0.25 | |
| 154 | A | 508.3 | <0.25 | |
| 155 | A | 594.4 | 0.55 | |
| 156 | D | 508.4 | 1.1 | |
| 157 | E | 521.4 | 115 | |
| 158 | D | 553.4 | <0.25 | |
| 159 | D | 545.1 | 0.79 | |
| 160 | A | 604.2 | <0.25 | |
| 161 | D | 548.0 | <0.25 | |
| 162 | F | 525.1 | 13 | |
| 163 | D | 520.8 | <0.25 | |
| 164 | E | 522.4 | 289 | |
| 165 | E | 852.4 | >100 nM | |
| 166 | D | 521.8 | 0.52 | |
| 167 | D | 526.8 | <0.25 | |
| 168 | A | 586.2 | 0.34 | |
| 169 | E | 526.0 | 24 | |
| 170 | A | 480.0 | <0.25 | |
| 171 | A | 526.4 | <0.25 | |
| 172 | D | 506.2 | 1.5 | |
| 173 | D | 541.2 | 0.41 | |
| 174 | D | 541.2 | <0.25 | |
| 175 | D | 524.1 | 0.51 | |
| 176 | D | 597.3 | 23 | |
| 177 | A | 526.0 | 0.39 | |
| 178 | D | 537.2 | <0.25 | |
| 179 | E | 526.4 | 1.6 | |
| 180 | F | 498.2 | 4.6 | |
| 181 | F | 538.2 | 0.75 | |
| 182 | F | 541.2 | 1.2 | |
| 183 | F | 538.1 | 0.49 | |
| 184 | D | 519.2 | 0.45 | |
| 185 | D | 532.2 | <0.25 | |
| 186 | F | 528.1 | 0.79 | |
| 187 | D | 508.1 | 0.91 | |
| 188 | D | 577.2 | 1.9 | |
| 189 | D | 565.2 | <0.25 | |
| 190 | D | 467.3 | 5.8 | |
| 191 | D | 510.2 | 15 | |
| 192 | D | 525.2 | 0.33 | |
| 193 | E | 465.2 | 2.5 | |
| 194 | D | 540.2 | 0.61 | |
| 195 | D | 560.3 | <0.25 | |
| 196 | D | 518.1 | <0.25 | |
| 197 | D | 549.1 | <0.25 | |
| 198 | E | 466.3 | 0.53 | |
| 199 | E | 556.3 | 5.8 | |
| 200 | E | 512.3 | <0.25 | |
| 201 | D | 507.1 | <0.25 | |
| 202 | D | 534.1 | 0.29 | |
| 203 | D | 481.0 | 2.9 | |
| 204 | E | 497.2 | 0.71 | |
| 205 | E | 553.1 | 0.16 | |
| 206 | D | 554.1 | <0.25 | |
| 207 | E | 527.0 | 2.6 | |
| 208 | E | 543.0 | 14 | |
| 209 | E | 523.2 | 0.67 | |
| 210 | E | 562.3 | <0.25 | |
| 211 | D | 563.3 | <0.25 | |
| 212 | D | 524.2 | <0.25 | |
| 213 | E | 525.1 | <0.25 | |
| 214 | E | 572.2 | 16 | |
| 215 | E | 511.3 | 0.31 | |
| 216 | D | 524.26 | 1.5 | |
| 217 | E | 538.4 | 1.9 | |
| 218 | D | 551.1 | 0.25 | |
| 219 | D | 550.1 | <0.25 | |
| 220 | D | 554.1 | 0.78 | |
| 221 | D | 538.3 | 0.98 | |
| 222 | D | 524.1 | <0.25 | |
| 223 | E | 521.3 | 0.29 | |
| 224 | D | 571.4 | 0.35 | |
| 225 | D | 484.0 | 0.56 | |
| 226 | D | 466.9 | 0.61 | |
| 227 | D | 540.4 | 6.9 | |
| 228 | D | 554.4 | <0.25 | |
| 229 | D | 557.4 | 1.4 | |
| 230 | D | 498.4 | <0.25 | |
| 231 | D | 539.4 | 1.6 | |
| 232 | D | 501.4 | <0.25 | |
| 233 | D | 558.1 | <0.25 | |
| 234 | D | 522.0 | 34 | |
| 235 | D | 513.2 | 19 | |
| 236 | D | 508.2 | 0.56 | |
| 237 | D | 527.4 | <0.25 | |
| 238 | E | 598.3 | 3.6 | |
| 239 | E | 512.2 | 18 | |
| 240 | E | 523.2 | 0.73 | |
| 241 | D | 524.2 | 0.33 | |
| 242 | D | 598.1 | 0.35 | |
| 243 | D | 456.0 | 15 | |
| 244 | D | 523.0 | <0.25 | |
| 245 | D | 513.3 | <0.25 | |
| 246 | D | 565.3 | 1.1 | |
| 247 | D | 468.3 | 2.9 | |
| 248 | D | 513.2 | <0.25 | |
| 249 | D | 553.3 | 0.77 | |
| 250 | D | 495.0 | 1.3 | |
| 251 | D | 452.1 | 0.45 | |
| 252 | D | 508.1 | <0.25 | |
| 253 | D | 510.2 | <0.25 | |
| 254 | D | 540.4 | 1.7 | |
| 255 | D | 552.4 | 0.26 | |
| 256 | D | 603.4 | 2.9 | |
| 257 | E | 497.0 | 26 | |
| 258 | D | 498.0 | 2.9 | |
| 259 | E | 486.1 | 1.2 | |
| 260 | D | 503.1 | 2.0 | |
| 261 | D | 494.3 | 0.62 | |
| 262 | D | 494.2 | 0.29 | |
| 263 | D | 495.3 | <0.25 | |
| 264 | D | 599.4 | <0.25 | |
| 265 | D | 511.8 | <0.25 | |
| 266 | A | 506.0 | 4.4 | |
| 267 | D | 526.0 | 6.4 | |
| 268 | G | 536.0 | 0.28 | |
| 269 | G | 452.0 | 0.38 | |
| 270 | D | 512.0 | 9.9 | |
| 271 | F | 524.0 | 0.64 | |
| 272 | D | 458.0 | 1.2 | |
| 273 | A | 505.0 | 1.8 | |
| 274 | A | 505.0 | 0.51 | |
| 275 | A | 505.0 | 3.1 | |
| 276 | D | 498.4 | <0.25 | |
| 277 | D | 510.4 | 3.2 | |
| 278 | D | 442.3 | <0.25 | |
| 279 | D | 412.3 | 1.5 | |
| 280 | D | 507.1 | <0.25 | |
| 281 | D | 543.1 | 20 | |
| 282 | D | 524.1 | 0.26 | |
| 283 | D | 551.4 | <0.25 | |
| 284 | D | 510.0 | 5.6 | |
| 285 | D | 511.2 | 4.3 | |
| 286 | D | 471.0 | 21 | |
| 287 | A | 503.2 | 0.93 | |
| 288 | D | 583.2 | <0.25 | |
| 289 | E | 461.0 | 0.88 | |
| 290 | D | 528.1 | 7.3 | |
| 291 | D | 462.1 | 0.35 | |
| 292 | D | 522.3 | 11 | |
| 293 | D | 498.0 | <0.25 | |
| 294 | D | 482.4 | 0.29 | |
| 295 | D | 509.4 | 2.7 | |
| 296 | D | 525.1 | 1.6 | |
| 297 | D | 542.1 | 0.69 | |
| 298 | D | 491.0 | 0.80 | |
| 299 | D | 396.1 | 0.60 | |
| 300 | D | 492.0 | 0.50 | |
| 301 | D | 526.2 | <0.25 | |
| 302 | D | 510.0 | 0.35 | |
| 303 | A | 505.0 | 1.1 | |
| 304 | A | 519.0 | 13 | |
| 305 | D | 493.7 | 0.38 | |
| 306 | D | 473.0 | 0.56 | |
| 307 | D | 491.0 | <0.25 | |
| 308 | D | 525.0 | 0.42 | |
| 309 | D | 525.1 | 0.99 | |
| 310 | D | 503.4 | 4.4 | |
| 311 | D | 530.2 | 8.1 | |
| 312 | D | 477.2 | 0.94 | |
| 313 | D | 453.0 | 0.87 | |
| 314 | D | 522.0 | 0.49 | |
| 315 | D | 538.0 | 4.5 | |
| 316 | D | 428.0 | 1.6 | |
| 317 | D | 532.3 | <0.25 | |
| 318 | A | 490.2 | 4.8 | |
| 319 | D | 555.88 | 7.5 | |
| 320 | D | 504.2 | <0.25 | |
| 321 | D | 452.3 | 0.41 | |
| 322 | A | 535.3 | 1.9 | |
| 323 | A | 428.2 | 4.5 | |
| 324 | D | 537.3 | 0.40 | |
| 325 | D | 530.4 | <0.25 | |
| 326 | D | 555.1 | 9.1 | |
| 327 | D | 430.2 | 4.1 | |
| 328 | D | 467.0 | 3.1 | |
| 329 | D | 446.0 | 4.1 | |
| 330 | D | 447.3 | 3.6 | |
| 331 | D | 576.2 | 24 | |
| 332 | D | 539.2 | 15 | |
| 333 | D | 525.2 | 3.8 | |
| 334 | D | 558.2 | 3.4 | |
| 335 | D | 442.8 | 4.3 | |
| 336 | D | 519.7 | 33 | |
| 337 | D | 519.7 | 9.2 | |
| 338 | D | 447.3 | 4.7 | |
| 339 | D | 412.1 | 3.4 | |
| 340 | D | 505.8 | 12 | |
| 341 | D | 476.0 | 3.2 | |
| 342 | D | 494.2 | 13 | |
| 343 | D | 491.0 | 8.4 | |
| 344 | D | 412.1 | 4.3 | |
| 345 | D | 485.2 | 3.7 | |
| 346 | D | 667.0 | 13 | |
| 347 | D | 555.2 | 3.4 | |
| 348 | A | 505.2 | 3.6 | |
| 349 | D | 543.7 | <0.25 | |
| 350 | D | 543.7 | <0.25 | |
| 351 | D | 521.8 | <0.25 | |
| 352 | D | 543.1 | <0.25 | |
| 353 | D | 553.1 | 0.49 | |
| 354 | D | 510.4 | 0.27 | |
| 355 | D | 578.2 | 0.30 | |
| 356 | D | 563.1 | <0.25 | |
| 357 | D | 567.8 | <0.25 | |
| 358 | D | 583.8 | <0.25 | |
| 359 | D | 553.1 | 1.7 | |
| 360 | D | 581.2 | 7.2 | |
| 361 | D | 539.4 | 1.9 | |
| 362 | D | 538.5 | 1.4 | |
| 363 | D | 538.2 | <0.25 | |
| 364 | D | 525.8 | 0.559 | |
| 365 | D | 537.1 | 100 | |
| 366 | D | 552.8 | 3 | |
| 367 | D | 568.2 | 0.27 | |
| 368 | D | 537.1 | 7.1 | |
| 369 | E | 546.42 | 0.31 | |
| 370 | E | 547.41 | 2.9 | |
| 371 | D | 533.1 | 8.1 | |
| 372 | D | 549.1 | 0.39 | |
| 373 | D | 509.35 | 4.6 | |
| 374 | E | 508.36 | >100 | |
| 375 | D | 511.4 | <0.25 | |
| 376 | E | 533.39 | 3.0 | |
| 377 | D | 527.1 | >100 | |
| 378 | D | 576.1 | <0.25 | |
| 379 | D | 527.1 | 18 | |
| 380 | D | 539.1 | 11 | |
| 381 | D | 537.1 | <0.25 | |
| 382 | D | 611.8 | <0.25 | |
| 383 | D | 537.2 | <0.25 | |
| 384 | D | 537.1 | <0.25 | |
| 385 | D | 524.8 | 0.87 | |
| 386 | D | 580.2 | <0.25 | |
| 387 | D | 534.7 | <0.25 | |
| 388 | D | 536.8 | <0.25 | |
| 389 | D | 538.8 | <0.25 | |
| 390 | D | 538.8 | <0.25 | |
| 391 | D | 510.35 | >100 | |
| 392 | E | 509.21 | >100 | |
| 393 | D | 572.0 | <0.25 | |
| 394 | D | 539.1 | <0.25 | |
| 395 | D | 565.2 | 0.25 | |
| 396 | D | 526.1 | 10 | |
| 397 | D | 526.1 | 0.39 | |
| 398 | D | 577.1 | 2.1 | |
| 399 | D | 591.2 | 1.5 | |
| 400 | D | 541.1 | 0.46 | |
| 401 | E | 523.2 | <0.25 | |
| 402 | D | 494.1 | 0.88 | |
| 403 | D | 525.1 | <0.25 | |
| 404 | D | 508.6 | <0.25 | |
| 405 | D | 508.1 | <0.25 | |
| 406 | D | 512.3 | 2.8 | |
| 407 | D | 451.2 | <0.25 | |
| 408 | D | 538.2 | 1.4 | |
| 409 | D | 523.5 | <0.25 | |
| 410 | D | 509.5 | 0.31 | |
| 411 | D | 507.2 | 0.37 | |
| 412 | D | 521.2 | 0.25 | |
| 413 | D | 539.2 | <0.25 | |
| 414 | D | 553.1 | <0.25 | |
| 415 | D | 566.2 | 0.25 | |
| 416 | D | 526.1 | 0.69 | |
| 417 | D | 493.2 | 3.7 | |
| 418 | D | 552.2 | 1.6 | |
| 419 | D | 533.1 | 0.72 | |
| 420 | D | 552.1 | 0.31 | |
| 421 | D | 522.1 | <0.25 | |
| 422 | D | 519.1 | 0.54 | |
| 423 | D | 583.5 | 0.40 | |
| 424 | D | 510.0 | 2.3 | |
| 425 | D | 524.2 | 0.30 | |
| 426 | D | 551.2 | 0.39 | |
| 427 | D | 552.2 | 0.27 | |
| 428 | D | 510.35 | <0.25 | |
| 429 | D | 547.3 | <0.25 | |
| 430 | D | 509.32 | <0.25 | |
| 431 | D | 535.41 | <0.25 | |
| 432 | D | 566.2 | <0.25 | |
| 433 | D | 459.1 | 6.2 | |
| 434 | D | 551.1 | <0.25 | |
| 435 | D | 536.1 | <0.25 | |
| 436 | D | 519.1 | <0.25 | |
| 437 | D | 535.1 | <0.25 | |
| 438 | D | 551.1 | <0.25 | |
| 439 | D | 561.1 | <0.25 | |
| 440 | D | 535.1 | <0.25 | |
| 441 | D | 554.1 | 0.43 | |
| 442 | D | 553.1 | <0.25 | |
| 443 | D | 551.1 | <0.25 | |
| 444 | D | 581.2 | <0.25 | |
| 445 | D | 589.1 | <0.25 | |
| 446 | D | 561.2 | <0.25 | |
| 447 | E | 560.2 | <0.25 | |
| 448 | D | 539.2 | <0.25 | |
| 449 | D | 539.1 | <0.25 | |
| 450 | D | 522.1 | 0.48 | |
| 451 | D | 550.1 | <0.25 | |
| 452 | D | 586.1 | <0.25 | |
| 453 | D | 536.2 | <0.25 | |
| 454 | D | 550.1 | 1.1 | |
| 455 | D | 555.1 | <0.25 | |
| 456 | D | 589.1 | <0.25 | |
| 457 | D | 525.1 | 0.37 | |
| 458 | D | 536.2 | 0.10 | |
| 459 | D | 555.1 | <0.25 | |
| 460 | D | 551.1 | <0.25 | |
| 461 | D | 539.1 | <0.25 | |
| 462 | D | 562.2 | <0.25 | |
| 463 | D | 526.1 | <0.25 | |
| 464 | E | 546.1 | <0.25 | |
| 465 | E | 565.2 | 0.27 | |
| 466 | D | 553.1 | <0.25 | |
| 467 | D | 549.2 | <0.25 | |
| 468 | D | 571.1 | <0.25 | |
| 469 | D | 580.2 | <0.25 | |
| 470 | D | 540.3 | 0.0492 | |
| 471 | D | 568.2 | 2.1 | |
| 472 | D | 549.2 | <0.25 | |
| 473 | C | 456.1 | 3.5 | |
| 474 | C | 447.1 | 7.6 | |
| 475 | A | 543.3 | 0.30 | |
| 476 | D | 549.2 | <0.25 | |
| 477 | D | 541.3 | 0.39 | |
| 478 | E | 544.3 | 0.50 | |
| 479 | E | 544.3 | 7.4 | |
| 480 | A | 535.6 | 1.7 | |
| 481 | D | 555.0 | <0.25 | |
| 482 | D | 485.3 | 0.73 | |
| 483 | D | 535.5 | <0.25 | |
| 484 | D | 521.6 | 0.32 | |
| 485 | D | 553.6 | 0.70 | |
| 486 | D | 541.5 | 0.36 | |
| 487 | D | 579.2 | <0.25 | |
| 488 | D | 536.5 | <0.25 | |
| 489 | D | 549.6 | 0.40 | |
| 490 | D | 551.2 | <0.25 | |
| 491 | D | 563.2 | <0.25 | |
| 492 | D | 564.2 | <0.25 | |
| 493 | D | 546.2 | <0.25 | |
| 494 | D | 579.2 | <0.25 | |
| 495 | D | 565.4 | 0.33 | |
| 496 | D | 579.4 | <0.25 | |
| 497 | D | 583.4 | 0.45 | |
| 498 | E | 540.4 | 0.63 | |
| 499 | D | 554.4 | <0.25 | |
| 500 | D | 496.3 | <0.25 | |
| 501 | D | 549.2 | 0.93 | |
| 502 | D | 541.4 | 2.4 | |
| 503 | D | 569.5 | <0.25 | |
| 504 | D | 561.2 | <0.25 | |
| 505 | D | 610.2 | <0.25 | |
| 506 | D | 605.6 | <0.25 | |
| 507 | D | 535.5 | 1.5 | |
| 508 | D | 591.6 | 1.2 | |
| 509 | D | 565.5 | 1.7 | |
| 510 | D | 592.1 | 0.92 | |
| 511 | D | 629.8 | 0.54 | |
| 512 | D | 532.1 | 8.0 | |
| 513 | D | 518.1 | 3.2 | |
| 514 | D | 529.0 | 7.7 | |
| 515 | D | 634.2 | 2.7 | |
| 516 | D | 636.2 | 0.39 | |
| 517 | D | 532.2 | 0.88 | |
| 518 | C | 495.4 | 1.4 | |
| 519 | D | 535.4 | 12.0 | |
| 520 | D | 580.1 | 0.27 | |
| 521 | D | 561.1 | 15.0 | |
| 522 | D | 606.1 | <0.25 | |
| 523 | D | 519.2 | <0.25 | |
| 524 | D | 535.3 | 2.3 | |
| 525 | D | 535.2 | 1.3 | |
| 526 | D | 606.2 | 1.5 | |
| 527 | G | 536.1 | <0.25 | |
| 528 | G | 579.2 | 0.44 | |
| 529 | G | 579.2 | 0.59 | |
| 530 | G | 564.1 | <0.25 | |
| 531 | D | 522.1 | 0.27 | |
| 532 | A | 511.2 | 1.7 | |
| 533 | D | 536.2 | 1.6 | |
| 534 | A | 579.2 | 0.95 | |
| 535 | D | 619.2 | 0.73 | |
| 536 | D | 618.2 | 0.96 | |
| 537 | D | 564.2 | 0.44 | |
| 538 | D | 565.2 | 5.7 | |
| 539 | A | 536.2 | 33.3 | |
| 540 | G | 564.5 | 1.0 | |
| 541 | G | 565.1 | 0.55 | |
| 542 | G | 565.2 | 0.29 | |
| 543 | G | 565.2 | 51 | |
| 544 | A | 529.1 | 1.9 | |
| 545 | A | 525.1 | 1.1 | |
| 546 | A | 535.4 | <0.25 | |
| 547 | A | 535.4 | <0.25 | |
| 548 | A | 619.2 | 0.79 | |
| 549 | G | 592.5 | 1.5 | |
| 550 | G | 549.4 | <0.25 | |
| 551 | A | 579.1 | 0.47 | |
| 552 | A | 579.1 | 0.45 | |
| 553 | G | 577.5 | 0.50 | |
| 554 | G | 507.4 | <0.25 | |
| 555 | G | 549.3 | 1.0 | |
| 556 | G | 549.4 | 1.8 | |
| 557 | D | 561.3 | <0.25 | |
| 558 | G | 563.3 | <0.25 | |
| 559 | G | 550.3 | <0.25 | |
| 560 | G | 577.4 | 1.15 | |
| 561 | G | 563.3 | >100 | |
| 562 | A | 539.3 | <0.25 | |
| 563 | A | 612.4 | <0.25 | |
| 564 | D | 624.1 | 0.38 | |
| 565 | A | 624.2 | 1.1 | |
| 566 | A | 565.4 | 2.0 | |
| 567 | A | 553.1 | 4.3 | |
| 568 | | 557.3 | 45 | |
| 569 | A | 547.3 | 5.5 | |
| 570 | G | 549.3 | <0.25 | |
| 571 | A | 551.3 | <0.25 | |
| 572 | G | 551.3 | <0.25 | |
| 573 | H | 551.2 | 1.0 | |
| 574 | A | 566.4 | 1.1 | |
| 575 | A | 589.3 | 0.32 | |
| 576 | | 543.0 | 29 | |
| 577 | A | 554.9 | <0.25 | |
| 578 | | 557.4 | 25 | |
| 579 | A | 594.0 | 0.33 | |
| 580 | A | 551.3 | 0.34 | |
| 581 | A | 595.4 | 0.99 | |
| 582 | A | 591.4 | 1.3 | |
| 583 | A | 591.3 | <0.25 | |
| 584 | A | 591.4 | 0.51 | |
| 585 | A | 537.3 | 0.47 | |
| 586 | A | 553.4 | 1.2 | |
| 587 | A | 565.4 | 0.69 | |
| 588 | A | 553.3 | 0.75 | |
| 589 | G | 549.0 | 0.70 | |
| 590 | A | 519.0 | <0.25 | |
| 591 | A | 555.0 | <0.25 | |
| 592 | A | 588.4 | 0.48 | |
| 593 | A | 535.3 | 9.9 | |
| 594 | A | 549.0 | 3.0 | |
| 595 | A | 549.0 | 2.9 | |
| 596 | G | 583.0 | 0.45 | |
| 597 | A | 537.0 | 3.6 | |
| 598 | G | 532.0 | 15 | |
| 599 | G | 546.0 | 21 | |
| 600 | G | 535.0 | 0.60 | |
| 601 | G | 532.0 | 2.7 | |
| 602 | | 515.3 | 11 | |
| 603 | | 556.3 | 40 | |
| 604 | | 529.3 | 30 | |
| 605 | A | 563.3 | 4.0 | |
| 606 | H | 541.0 | 4.0 | |
| 607 | A | 599.3 | 0.95 | |
| 608 | A | 545.3 | 0.79 | |
| 609 | H | 595.0 | 3.1 | |
| 610 | D | 593.3 | 0.27 | |
| 611 | D | 634.4 | 1.2 | |
| 612 | D | 620.1 | 0.85 | |
| 613 | D | 618.4 | 0.35 | |
| 614 | D | 606.1 | 0.40 | |
| 615 | E | 619.3 | 1.1 | |
| 616 | A | 579.0 | 0.35 | |
| 617 | A | 593.0 | <0.25 | |
| 618 | H | 539.3 | 0.81 | |
| 619 | H | 500.3 | 4.9 | |
| 620 | H | 514.3 | 3.2 | |
| 621 | | 581.3 | 6.3 | |
| 622 | H | 580.1 | 1.4 | |
| 623 | A | 561.3 | <0.25 | |
| 624 | A | 555.2 | 1.1 | |
| 625 | H | 500.4 | 0.42 | |
| 626 | A | 620.4 | 3.5 | |
| 627 | A | 618.3 | 0.30 | |
| 628 | G | 509.4 | <0.25 | |
| 629 | A | 551.0 | 1.0 | |
| 630 | H | 580.1 | 8.5 | |
| 631 | H | 580.1 | 0.93 | |
| 632 | A | 536.0 | 0.35 | |
| 633 | A | 581.4 | 0.95 | |
| 634 | H | 610.0 | 0.86 | |
| 635 | A | 594.3 | 0.25 | |
| 636 | A | 610.1 | 0.33 | |
| 637 | A | 549.4 | <0.25 | |
| 638 | A | 552.9 | <0.25 | |
| 639 | A | 553.0 | 0.47 | |
| 640 | A | 610.0 | 0.54 | |
| 641 | A | 608.0 | <0.25 | |
| 642 | A | 526.4 | 0.72 | |
| 643 | A | 608.0 | 0.30 | |

### Reversible CYP Inhibition

A seven-point semi-log dilution of each test compound (up to 30 μM) or positive control inhibitors for CYP2C9 (up to 1000 nM sulphenazole), CYP2D6 (up to 500 nM quinidine) and CYP3A4 (up to 250 nM ketoconazole) were dissolved in DMSO and added to the incubation plate using a Tecan 300 (normalized for 0.3% DMSO content). Final reaction conditions were: human liver microsomes (0.1 mg/mL), potassium phosphate buffer 100 mM pH 7.4, 1 mM magnesium chloride, 5 μM diclofenac, 5 μM dextromethorphan and 2.5 μM midazolam. After a pre-incubation at 37 °C. The reaction was started with the addition of NADPH solution at the final concentration of 1 mM and carried out at 37 °C for 5 minutes. The reaction was stopped by the addition of 1 volume of cold acetonitrile with internal standard (labetalol) to the incubation plate. The incubation plate was centrifuged at 3000 rpm for 10 minutes to precipitate proteins, and the supernatant was analyzed by LC-MS/MS. Metabolite area ratio (4-OH-diclofenac, dextrorphan and 1-OH-midazolam) versus ISTD area ratio was used as the quantitative signal. Data were analyzed using the plot log of concentration (x-axis) versus the percentage of inhibition (y axis) and the IC₅₀, Hill Slope and R² were determined. The results are summarized in Table 4.

### PXR Method

An expression vector harboring a full-length PXR nuclear receptor plus the appropriate enhancers and promoters linked to the luciferase reporter gene were integrated into the tumor cells. Tumor cells transfected with the species-specific nuclear receptor and the corresponding response elements were seeded in a 96-well plate. Twenty-four hours after seeding, the cells were treated with six distinct concentrations (0.03-10 μM) of the test compound. The cells were then returned to the incubator for an additional 24 h. After this incubation period, the number of viable cells/well were determined using Promega's Cell Titer Fluor cytotoxicity assay. At the end of the assay, Promega's ONE-Glo was added to assess the receptor activation by monitoring reporter gene activity, and by comparing the results to vehicle-treated cells. Activation data were normalized to the number of viable cells/well. Results are expressed as a percentage of the response given by Rifampicin, the positive control, at a 10 μM dose. The results are summarized in Table 4.

### TDI Method

Test compound or positive control inhibitors (CYP2C9: Tienilic acid; CYP2D6: paroxetine; CYP3A4: mifepristone) were dissolved in DMSO and a semi-log dilution (up to 50.0 μM) was prepared to 2 Incubation plates and normalized for a final DMSO content of 0.5%. Human liver microsomes were diluted at 1 mg/mL with 100 mM potassium phosphate buffer pH 7.4 (1 mM MgCl₂) and added to both incubation plates. After pre-incubation at 37 °C, the reaction was started with the addition of NADPH (+NADPH condition) or with water (−NADPH condition). Plates were incubated for 30 min and 8 μL were transferred to a second incubation plate containing 72 μL of 1 mM NADPH, 20 μM diclofenac (CYP2C9), 20 μM dextromethorphan (CYP2D6) and 10 μM midazolam (CYP3A4) in 100 mM potassium phosphate buffer pH 7.4 (1 mM MgCl₂). Plates were incubated for 10 min and the reaction was stopped by the addition of cold acetonitrile containing internal standard (1:1 v/v). Incubation plates were centrifuged at 3000 rpm for 10 minutes to precipitate protein, and the supernatant was used for LC-MS/MS analysis. Metabolite area ratio (4-OH-diclofenac, dextrorphan and 1-OH-midazolam) versus internal standard area ratio was used as the quantitative signal. Data were analyzed using the plot log of concentration (x axis) versus the percentage of inhibition (y axis) and IC₅₀, Hill Slope and R² were determined. The IC₅₀ shift was then calculated by dividing the IC₅₀−NADPH condition by the IC₅₀ obtained in the +NADPH condition. The results are summarized in Table 4.

### Liver Microsomal Stability Assay

Liver microsomes were thawed on ice prior to use. The incubation mixtures were prepared in 96-well plates and contained test compound or control (1 μM), liver microsomes (0.5 mg of microsomal protein/mL), MgCl₂ (5 mM), phosphate buffer (100 mM, pH 7.4), 0.01% DMSO and 1% acetonitrile. A T=0 aliquot was withdrawn after 10 min pre-incubation at 37 °C on a shaker. Reactions were initiated by the addition of NADPH (final concentration of 1 mM) and the plate was kept on a shaker at 37 °C, further samples were withdrawn at 5, 15, 30, and 60 minutes. At each timepoint, the reactions were immediately terminated by adding of ice-cold acetonitrile containing internal standard to the withdrawn sample. The extracted samples were centrifuged for 10 minutes at 3200 rpm to pellet the precipitated microsomal protein, and the supernatant was combined 1:1 with water and analyzed by LC-MS/MS. Using the T=0 peak area ratio as 100%, the percentage of parent compound remaining was calculated. For each compound, the ln percentage remaining versus incubation time was plotted and the slope of this linear regression (−k) was converted to an in vitro T_{1/2} value and CLint using the equations below.

CLint (μL/min/mg mic protein)=−Kₑ*V

V=volume of incubation/amount of protein=(1000 μL/0.5 mg)=2000 μL/mg

Kₑ=elimination rate constant (slope of semi-natural log curve)

T_{1/2}=ln(2)/Kₑ

The results are summarized in Table 5.

TABLE 5

| | | | Time- | |
|---|---|---|---|---|
| | | | dependent | |
| | | Human | CYP3A4 | PXR |
| | | Microsomal | inhibition- | activation |
| | CYP3A4 | Stability: | shift | (% Max |
| | inhibition | CLint | after 30 | activity/ |
| | IC50 | (μL/min/ | min with | Max fold |
| Compound | (μM) | mg) | NADPH | induction) |
| 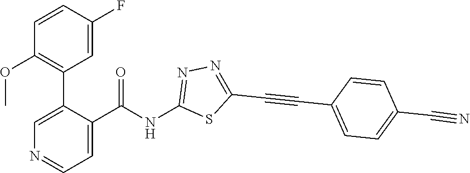 [SMILES: COc1ccc(F)cc1-c1cnccc1C(=O)Nc1nnc(C#Cc2ccc(C#N)cc2)s1] | 1.11 | 46.4 | 1x | 2.0/1.3x |
| 473 | | | | |
| 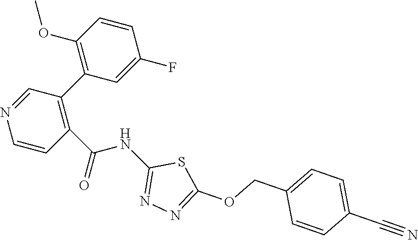 [SMILES: COc1ccc(F)cc1-c1cnccc1C(=O)Nc1nnc(OCc2ccc(C#N)cc2)s1] | 0.234 | 174 | 1x | 17/3.9x |
| 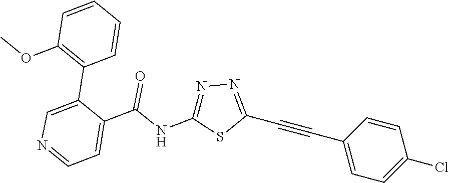 [SMILES: COc1ccccc1-c1cnccc1C(=O)Nc1nnc(C#Cc2ccc(Cl)cc2)s1] | 1.94 | 22.5 | 1x | 3.0/1.5x |
| 474 | | | | |
| 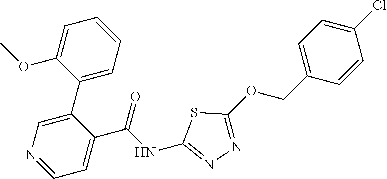 [SMILES: COc1ccccc1-c1cnccc1C(=O)Nc1nnc(OCc2ccc(Cl)cc2)s1] | 1.17 | 262 | >6.5x | 46.0/8.8 |
| 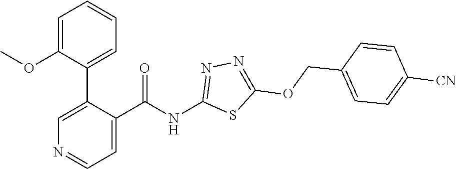 [SMILES: COc1ccccc1-c1cnccc1C(=O)Nc1nnc(OCc2ccc(C#N)cc2)s1] | 0.45 | 234 | 3.6x | 36.0/7.9 |
| 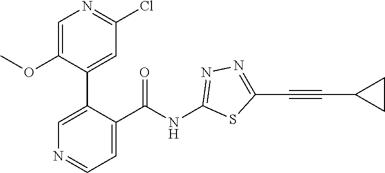 [SMILES: COc1cnc(Cl)cc1-c1cnccc1C(=O)Nc1nnc(C#CC2CC2)s1] | >30 | <7.7 | 1x | 5.0/1.8 |

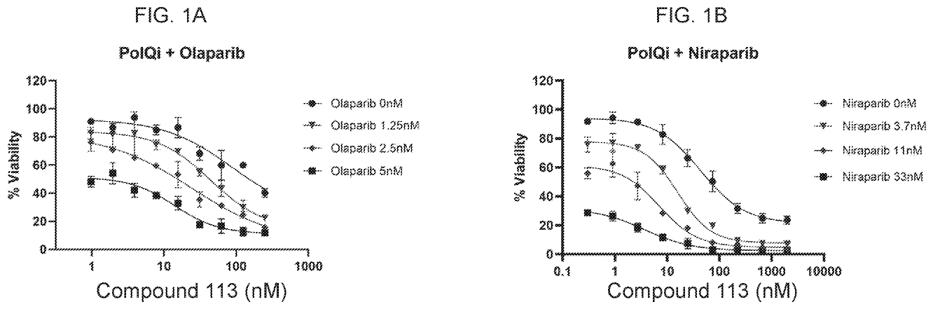

Combination therapy of Compound 113 and PARP Inhibitors in HCT116 BRCA2−/− cells HCT116-BRCA2−/− (750 cells/well) cells were seeded in a 96-well plate (Costar 3595) and incubated overnight in a tissue culture incubator (5% CO₂, 37 °C). The next day, Compound 113 and PARP inhibitors (olaparib or niraparib) were added using a TECAN D300 instrument. Media was replenished with fresh compounds every 3 or 4 days. Cellular growth was monitored using the Incucyte® Live-Cell Analysis System until DMSO-treated wells reached 90% confluency (10-12 days). Cellular confluency was determined using Incucyte® Cell-by-Cell Analysis Software Module and dose-response curves calculated using GraphPad Prism software. FIG. 1A shows the percent viability of HCT116 BRCA2−/− cells as a function of Compound 113 concentration and olaparib concentration. FIG. 1B shows the percent viability of HCT116 BRCA2−/− cells as a function of Compound 113 concentration and niraparib concentration. The results demonstrated that Compound 113 and a PARP inhibitor (olaparib or niraparib) when administered together was superior at reducing cancer cell viability compared to the inhibitors administered alone.

### Effect of Polθ-PARP Inhibitor Combination Therapy on HCT116 BRCA2−/− Tumor Volume in Mice

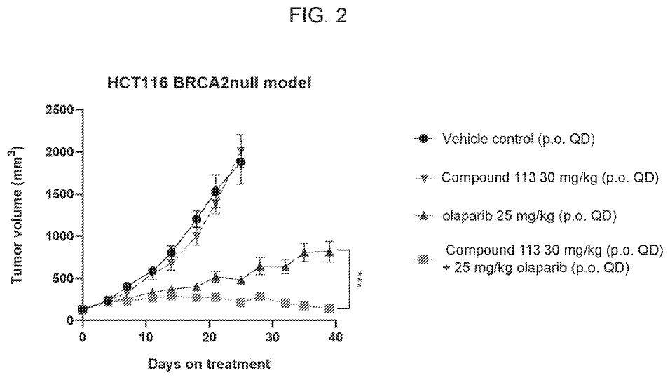

HCT116-BRCA2−/− (10⁷ cells/mouse) cells were injected subcutaneously in SCID-Beige mice. Mice were randomized at a mean tumor volume of 150 mm³. Compound 113 (30 mg/kg as an SDD suspension in 0.5% methocel+0.02% SLS) and PARP inhibitor olaparib (25 mg/kg) were dosed orally (p.o.) QD throughout the study. Control mice were dosed QD with vehicle solution. Tumor volumes measured (caliper) every 3-4 days. N=10, Welch's t test, *** p=0.0008. The results demonstrated that Compound 113 and Olaparib when administered together was superior at reducing tumor volume compared to the inhibitors alone (FIG. 2).

### Effect of Polθ-DNA-PK Inhibitor Combination Therapy on MDAMB436 BRCA1 Mutant Breast Tumor Cells

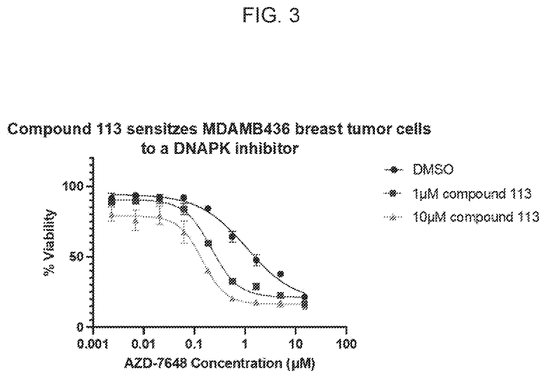

MDAMB436 BRCA1 mutant breast tumor cells were plated at 1500 cells/well in 96 well plates. Compounds were added on day 1 and cells were grown in at 37 °C for 9 days. Cell growth was assessed by the Incucyte assay. Cell viability was normalized to untreated cells. The results demonstrated that the combination of Compound 113 and AZD-7648 (DNA-PK inhibitor) was superior at reducing MDAMB436 BRCA1 cell viability compared to AZD-7648 alone (FIG. 3).

### Effect of Polθ-DNA-PK Inhibitor Combination Therapy on DLD1 BRCA2 Null Tumor Cells

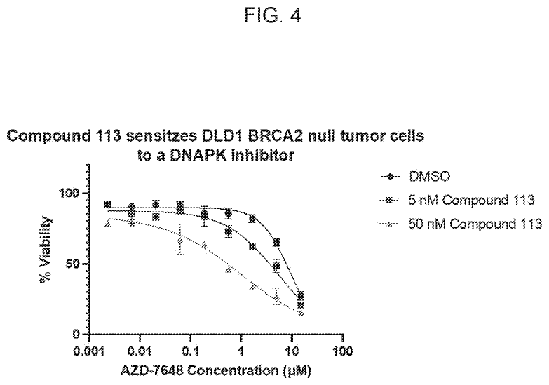

DLD1 BRCA2 null tumor cells were plated at 1000 cells/well in 96 well plates. Compounds were added on day 1 and cells were grown in at 37 °C for 8 days. Cell growth was assessed by the Incucyte assay. Cell viability was normalized to untreated cells. The results demonstrated that the combination of Compound 113 and AZD-7648 (DNA-PK inhibitor) was superior at reducing DLD1 BRCA2 null tumor cell viability compared to AZD-7648 alone (FIG. 4).

### Effect of Compound 113 and Radiotherapy in DOTC24510 BRCA 2 Mutant Breast Tumor Cells

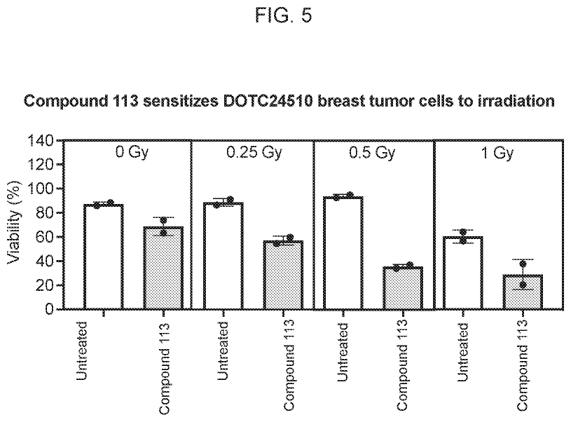

DOTC24510 BRCA 2 mutant breast tumor cells were plated in 96 well plates at a density of 1000 cells per well and cultured in tissue culture medium at 37 °C. On day 1, cells were treated with either DMSO or 200 nM of Compound 113. Cells were irradiated with 0, 0.25, 0.5 or 1 Gy of radiation on days 2, 6, and 9. Cell viability was assessed by Incucyte assay on day 13. The results show that the combination of Compound 113 and radiotherapy (at 0.5 or 1 Gy) is superior at reducing DOTC24510 BRCA 2 mutant breast tumor cell viability compared to Compound 113 alone (FIG. 5).

### Effect of Compound 113 and Carboplatin in DLD1-BRCA2−/− Tumors in Mice

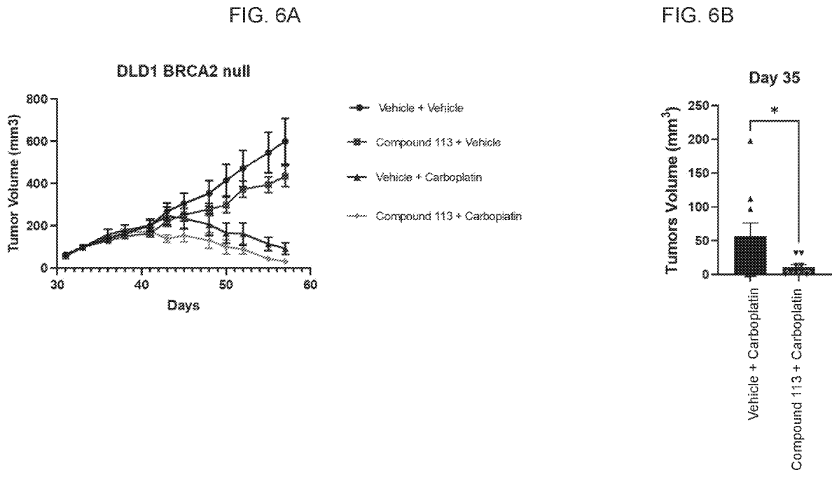

DLD1-BRCA2−/− (10⁷ cells/mouse) cells were injected subcutaneously in SCID-Beige mice. Mice were randomized at a mean tumor volume of 150 mm³. Compound 113 (45 mg/kg as an SDD suspension in 0.5% methocel+0.02% SLS) was dosed orally (p.o.) 0D throughout the study. Carboplatin (25 mg/kg) was dosed i.p. QW. Control mice were dosed QD and QW with corresponding vehicle solutions. Tumor volumes were measured (caliper) every 2-3 days. The results demonstrated that the “Compound 113 and carboplatin” combination therapy was superior at reducing tumor volume in mice compared to the monotherapy treatments (FIG. 6A). FIG. 6B shows the statistical analysis between the “vehicle and carboplatin” combination and the “Compound 113 and carboplatin” combination at day 35 (N=10, Welch's t test, * p=0.0428).

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1.-22. (canceled)

23. A compound selected from:

| No. | Structure |
|---|---|
| 431 | 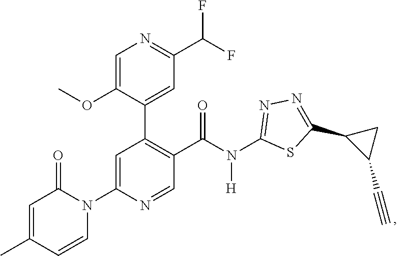 [SMILES: C#C[C@H]1C[C@@H]1c1nnc(NC(=O)c2cnc(-n3ccc(C)cc3=O)cc2-c2cc(C(F)F)ncc2OC)s1] |
| 546 | 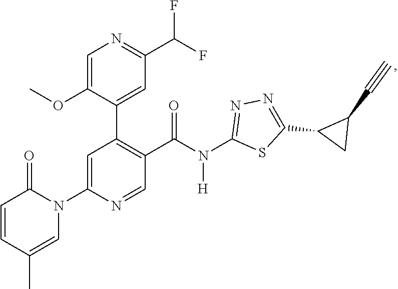 [SMILES: C#C[C@H]1C[C@@H]1c1nnc(NC(=O)c2cnc(-n3cc(C)ccc3=O)cc2-c2cc(C(F)F)ncc2OC)s1] |
| 547 | 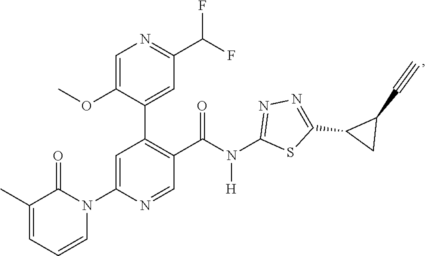 [SMILES: C#C[C@H]1C[C@@H]1c1nnc(NC(=O)c2cnc(-n3cccc(C)c3=O)cc2-c2cc(C(F)F)ncc2OC)s1] |
| 562 | 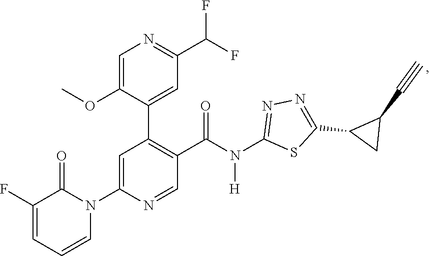 [SMILES: C#C[C@H]1C[C@@H]1c1nnc(NC(=O)c2cnc(-n3cccc(F)c3=O)cc2-c2cc(C(F)F)ncc2OC)s1] |
| 571 | 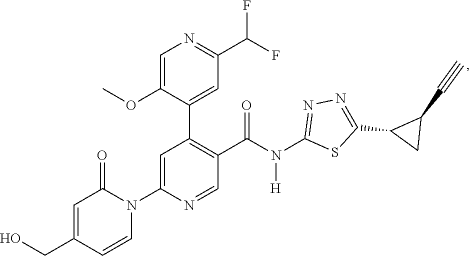 [SMILES: C#C[C@H]1C[C@@H]1c1nnc(NC(=O)c2cnc(-n3ccc(CO)cc3=O)cc2-c2cc(C(F)F)ncc2OC)s1] |
| | and |
| 591 | 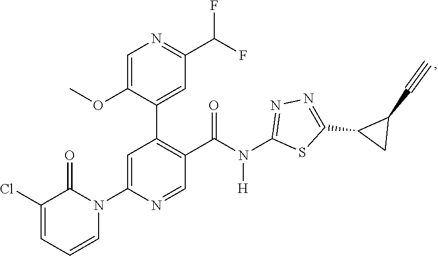 [SMILES: C#C[C@H]1C[C@@H]1c1nnc(NC(=O)c2cnc(-n3cccc(Cl)c3=O)cc2-c2cc(C(F)F)ncc2OC)s1] |

or a pharmaceutically acceptable salt thereof.

24. A pharmaceutical composition comprising a compound of claim 23, or a pharmaceutically acceptable salt thereof, and pharmaceutically acceptable excipient.
